(19) United States
(12) Patent Application Publication
(10) Pub. No.: US 2025/0011789 A1
(43) Pub. Date: Jan. 9, 2025

(54) Modified Short Interfering Nucleic Acid (siNA) Molecules and Uses Thereof

(71) Applicants: Aligos Therapeutics, Inc., South San Francisco, CA (US); Merck Sharp & Dohme LLC, Rahway, NJ (US)

(72) Inventors: N. Tilani S. De Costa, South San Francisco, CA (US); Xuan Luong, Oakland, CA (US); Aneerban Bhattacharya, South San Francisco, CA (US); Leonid Beigelman, San Mateo, CA (US); Jerome Deval, El Granada, CA (US); Saul Martinez Montero, Newton, MA (US)

(21) Appl. No.: 18/690,562

(22) PCT Filed: Sep. 8, 2022
(86) PCT No.: PCT/US2022/042923

§ 371 Date: Mar. 8, 2024

Related U.S. Application Data
(60) Provisional application No. 63/241,940, filed on Sep. 8, 2021.

(51) Int. Cl. C12N 15/113; A61K 45/06; A61P 1/16

(52) U.S. Cl. CPC C12N 15/1137; A61K 45/06; A61P 1/16; C12N 2310/11; C12N 2310/14; C12N 2310/312; C12N 2310/315; C12N 2310/321; C12N 2310/322; C12N 2310/351

(57) ABSTRACT

Disclosed herein are short interfering nucleic acid (siNA) molecules comprising modified nucleotides and uses thereof. The siNA molecules may be double stranded and comprise modified nucleotides selected from 2′—O-methyl nucleotides and 2′-fluoro nucleotides. Further disclosed herein are siNA molecules comprising additional modification including a phosphorylation blocker, conjugated moiety, or 5′-stabilized end cap. The siNA molecules may reduce or inhibit the production of hydroxysteroid dehydrogenase.

This application claims the priority of U.S. Provisional Patent Application No. U.S. 63/241,940, entitled “MODIFIED SHORT INTERFERING NUCLEIC ACID (SINA) MOLECULES AND USES THEREOF”, filed Sep. 8, 2021, which is incorporated herein by reference in its entirety for all purposes.

### FIELD OF THE DISCLOSURE

Described are short interfering nucleic acid (siNA) molecules comprising modified nucleotides, compositions, and uses thereof.

### BACKGROUND

RNA interference (RNAi) is a biological response to double-stranded RNA that mediates resistance to both endogenous parasitic and exogenous pathogenic nucleic acids and regulates the expression of protein-coding genes. The short interfering nucleic acids (siNA), such as siRNA, have been developed for RNAi therapy to treat a variety of diseases. For instance, RNAi therapy has been proposed for the treatment of metabolic diseases, neurodegenerative diseases, cancer, and pathogenic infections (See e.g., Rondindone, Biotechniques, 2018, 40 (4S), doi.org/10.2144/000112163, Boudreau and Davidson, Curr Top Dev Biol, 2006, 75:73-92, Chalbatani et al., Int J Nanomedicine, 2019, 14:3111-3128, Arbuthnot, Drug News Perspect, 2010, 23 (6): 341-50, and Chernikov et. al., Front. Pharmacol., 2019, doi.org/10.3389/fphar.2019.00444, each of which are incorporated by reference in their entirety). However, major limitations of RNAi therapy are the ability to effectively deliver siRNA to target cells and the degradation of the siRNA.

Non-alcoholic fatty liver disease (NAFLD) is an emerging global health problem and a potential risk factor for type 2 diabetes, cardiovascular disease, and chronic kidney disease. Nonalcoholic steatohepatitis (NASH), an advanced form of NAFLD, is a predisposing factor for development of cirrhosis and hepatocellular carcinoma. The increasing prevalence of NASH emphasizes the need for novel therapeutic approaches. 17ß-Hydroxysteroid dehydrogenase type 13 also known as 17ß-HSD type 13 (or HSD17B13) is an enzyme that is enriched in hepatocytes, where it localizes to subcellular lipid droplets. HSD17B13 is significantly up-regulated in the liver of patients with NAFLD and NASH and enhances lipogenesis. The role of HSD17B13 in lipogenesis appears to be mediated by its retinoid dehydrogenase activity. Reduction in HSD17B13 protein levels could lead to decreased levels of ALT and AST and improved liver histology of NAFLD and NASH.

The present disclosure provides siNA molecules that target HSD17B13 to reduce or inhibit the production of a hydroxysteroid dehydrogenase. The siNA molecules comprise optimized combinations and numbers of modified nucleotides, nucleotide lengths, design (e.g., blunt ends or overhangs, internucleoside linkages, conjugates), and modification patterns that exhibit improved delivery and stability.

### SUMMARY

One aspect of the present disclosure pertains to a double-stranded short interfering nucleic acid (siNA) molecule comprising a sense strand comprising a nucleotide sequence that is at least about 60%, 65%, 70%, 75%, 80%, 85%, 90%, 95%, or 100% identical to a nucleotide sequence of any one SEQ ID NOs: 1-100, 201-230, 262-287, 314-445, 576-603 or 638; and/or an antisense strand comprising a nucleotide sequence that is at least about 60%, 65%, 70%, 75%, 80%, 85%, 90%, 95%, or 100% identical to a nucleotide sequence of any one of SEQ ID NOs: 101-200, 231-260, 288-313, 446-575, 604-637 or 639-644, wherein the siNA molecule downregulates expression of a hydroxysteroid 17-beta dehydrogenase 13 (HSD17B13) gene.

Another aspect of the present disclosure pertains to a double-stranded short interfering nucleic acid (siNA) molecule comprising a sense strand comprising a nucleotide sequence of any one SEQ ID NOs: 1-100, 201-230, 262-287, 314-445, 576-603 or 638; and/or an antisense strand comprising a nucleotide sequence of any one of SEQ ID NOs: 101-200, 231-260, 288-313, 446-575, 604-637 or 639-644, wherein the siNA molecule downregulates expression of a hydroxysteroid 17-beta dehydrogenase 13 (HSD17B13) gene.

Another aspect of the present disclosure pertains to a double-stranded short interfering nucleic acid (siNA) molecule selected from any one of siNA Duplex ID Nos. D1-D178 or MD1-MD178.

Another aspect of the present disclosure pertains to a pharmaceutical composition comprising any of the siNA molecules according to the disclosure and a pharmaceutically acceptable carrier.

Another aspect of the present disclosure pertains to a method of treating a HSD17B13-associated disease in a subject in need thereof, comprising administering to the subject an amount of any of the siNA molecules or pharmaceutical compositions according to the disclosure, thereby treating the subject. For example, the liver disease may be NAFLD, hepatocellular carcinoma (HCC), and/or NASH and/or fatty liver.

Another aspect of the present disclosure pertains to a method of treating a liver disease in a subject in need thereof, comprising administering to the subject an amount of any of the siNA molecules or pharmaceutical compositions according to the disclosure, thereby treating the subject. For example, the liver disease may be NAFLD, HCC and/or NASH and/or fatty liver.

Another aspect of the present disclosure pertains to a method of treating a liver disease in a subject in need thereof, comprising administering to the subject an amount of any of the siNA molecules or pharmaceutical compositions according to the disclosure, further comprising administering to the subject at least one additional active agent, thereby treating the subject, wherein the at least one additional active agent is a liver disease treatment agent.

Another aspect of the present disclosure pertains to a method of reducing the expression level of HSD17B13 in a patient in need thereof comprising administering to the patient an amount of any of the siRNA molecules or pharmaceutical compositions according to the disclosure, thereby reducing the expression level of HSD17B13 in the patient.

The present technology provides a short interfering nucleic acid (siNA) molecule comprising: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O methyl nucleotide and a 2′-fluoro nucleotide, wherein at least one modified nucleotide is a 2′-O methyl nucleotide and the nucleotide at position 3, 5, 7, 8, 9, 10, 11, 12, 14, 17, and/or 19 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

The present technology also provides a molecule represented by Formula (VIII): 5′-Aₙ¹Bₙ²Aₙ³Bₙ⁴Aₙ⁵Bₙ⁶Aₙ⁷Bₙ⁸Aₙ⁹-3′ 3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′ wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O-methyl nucleotide or a nucleotide comprising a 5′-stabilized end cap or a phosphorylation blocker; B is a 2′-fluoro nucleotide; C represents overhanging nucleotides and is a 2′-O-methyl nucleotide, deoxy nucleotide, or uracil; n¹=1-6 nucleotides in length; each n², n⁶, n⁸, q³, q⁵, q⁷, q⁹, q¹¹, and q¹² is independently 0-1 nucleotides in length; each n³ and n⁴ is independently 1-3 nucleotides in length; n⁵ is 1-10 nucleotides in length; n⁷ is 0-4 nucleotides in length; each n⁹, q¹, and q² is independently 0-2 nucleotides in length; q⁴ is 0-3 nucleotides in length; q⁶ is 0-5 nucleotides in length; q⁸ is 2-7 nucleotides in length; and q¹⁰ is 2-11 nucleotides in length.

In any embodiment, the first nucleotide sequence comprises a nucleotide sequence of any one of SEQ ID Nos: 1-100, 201-230, 262-287, 314, or 315 and/or the second nucleotide sequence comprises a nucleotide sequence of any one of SEQ ID NOs: 101-200, 231-260, or 288-313. In any embodiment, the first nucleotide sequence comprises a nucleotide sequence of any one of SEQ ID Nos: 316-445, 576-603 or 638 and/or the second nucleotide sequence comprises a nucleotide sequence of any one of SEQ ID NOs: 446-575, 604-637 or 639-644.

In any embodiment, the siNA reduces or inhibits the production of a hydroxysteroid dehydrogenase. In any embodiment, the siNA decreases expression or activity of HSD17B13.

In any embodiment, the sense and/or antisense strands disclosed herein may further include a TT sequence adjacent to the first and/or second nucleotide sequence. In any embodiment, the sense and/or antisense strands disclosed herein may further include phosphorothioate internucleoside linkage(s), mesyl phosphoroamidate internucleoside linkage(s), 5′ stabilizing end cap(s), phosphorylation blocker(s), galactosamine(s), conjugated moiety or moieties as disclosed herein, destabilizing nucleotide(s) disclosed herein, modified nucleotide(s) disclosed herein, thermally destabilizing nucleotide(s), or a combination of two or more thereof. In some embodiments, the 5′ stabilizing end cap(s), the phosphorylation blocker(s), the conjugated moiety or moieties as disclosed herein, the galactosamine(s), the destabilizing nucleotide(s) disclosed herein, the modified nucleotide(s) disclosed herein, the thermally destabilizing nucleotide(s), or a combination of two or more thereof are attached to the sense and/or antisense strand via one or more linkers independently selected from a phosphodiester linker, phosphorothioate linker, or phosphorodithioate linker.

Further disclosed herein are compositions and medicaments comprising any of the siNAs disclosed herein.

In any embodiment, the siNA molecule, compositions, and/or medicaments disclosed herein may be used in the treatment of a disease such as a liver disease. In any embodiment, the liver disease may include nonalcoholic fatty liver disease (NAFLD), hepatocellular carcinoma (HCC), or nonalcoholic steatohepatitis (NASH).

### BRIEF DESCRIPTION OF THE DRAWINGS

FIG. 1 illustrates an exemplary siNA molecule.

FIG. 2 illustrates an exemplary siNA molecule.

FIGS. 3A-3H illustrate exemplary double-stranded siNA molecules.

FIG. 4 and FIG. 5 illustrate HSD17B13 mRNA knockdown by modified siNA duplexes according to the present disclosure at 7 days post dose.

FIG. 6 illustrates HSD17B13 mRNA knockdown by modified siNA duplexes according to the present disclosure at 7 days post dose at 1.5 mpk and at 7 days post dose.

FIG. 7 illustrates HSD17B13 mRNA knockdown by modified siNA duplexes according to the present disclosure.

FIG. 8 illustrates HSD17B13 mRNA knockdown by modified siNA duplexes according to the present disclosure.

FIG. 9 illustrates HSD17B13 mRNA knockdown by modified siNA duplexes according to the present disclosure.

FIG. 10 illustrates HSD17B13 mRNA knockdown by modified siNA duplexes according to the present disclosure.

FIG. 11 illustrates western blot showing HSD17B13 protein knockdown by ds-siNA 137, ds-siNA 144, ds-siNA 148, and ds-siNA 151 at 7 days post dose.

FIG. 12 illustrates quantitation of western blot from FIG. 11.

FIG. 13 illustrates HSD17B13 mRNA knockdown by ds-siNA 137, ds-siNA 144, ds-siNA 148, and ds-siNA 151 at 7 days post dose.

FIG. 14 illustrates western blot showing HSD17B13 protein knockdown by ds-siNA 137, ds-siNA 144, ds-siNA 148, and ds-siNA 151 at 14 days post dose.

FIG. 15 illustrates quantitation of western blot from FIG. 14.

FIG. 16 illustrates HSD17B13 mRNA knockdown by ds-siNA 137, ds-siNA 144, ds-siNA 148, and ds-siNA 151 at 14 days post dose.

FIG. 17 illustrates HSD17B13 mRNA knockdown by modified siNA duplexes according to the present disclosure.

FIG. 18 illustrates HSD17B13 mRNA knockdown by modified siNA duplexes according to the present disclosure.

FIG. 19 illustrates HSD17B13 mRNA knockdown by modified siNA duplexes according to the present disclosure.

FIG. 20 illustrates HSD17B13 mRNA knockdown by modified siNA duplexes according to the present disclosure.

### DETAILED DESCRIPTION

This section presents a detailed description of the many different aspects and embodiments that are representative of the disclosure. This description is by way of several exemplary illustrations of varying detail and specificity. Other features and advantages of these embodiments are apparent from the additional descriptions provided herein, including the different examples. The provided examples illustrate different components and methodology useful in practicing various embodiments of the disclosure. The examples are not intended to limit the claimed disclosure. Based on the present disclosure, the ordinarily skilled artisan can identify and employ other components and methodology useful for practicing the present disclosure.

The present disclosure will be better understood with reference to the following definitions.

### Definitions

Unless defined otherwise, all technical and scientific terms used herein have the meaning commonly understood by a person of ordinary skill in the art to which this disclosure belongs.

The terms “a” and “an” as used herein mean “one or more” and include the plural unless the context is inappropriate.

The term “about” as used herein when referring to a measurable value (e.g., weight, time, and dose) is meant to encompass variations, such as ±10%, ±5%, ±1%, or ±0.1% of the specified value.

Except where otherwise indicated, all numbers expressing quantities of ingredients, reaction conditions, and so forth used in the specification and claims are to be understood as being modified in all instances by the term “about,” whether or not the term “about” is present in front of the number. Accordingly, unless indicated to the contrary, the numerical parameters set forth in the following specification and attached claims are approximations that may vary depending upon the desired properties sought to be obtained by the present disclosure. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 50, 7, 34, 46.1, 23.7, or any other value or range within the range. Moreover, as used herein, the term “at least” includes the stated number, e.g., “at least 50” includes 50.

As a general matter, compositions specifying a percentage are specifying a percentage by weight unless otherwise specified. Further, if a variable is not accompanied by a definition, then the previous definition of the variable controls.

The term “including” is used herein to mean, and is used interchangeably with, the phrase “including, but not limited to”.

As used herein, the terms “siRNA” and “siRNA molecule” and “siNA” and “siNA molecule” are used interchangeably and refer to short (or small) interfering ribonucleic acid (RNA), including chemically modified RNA, which may be single-stranded or double-stranded. As used herein, the siRNA may comprise modified nucleotides, including modifications at the sugar, nucleobase, and/or phosphodiester backbone (internucleoside linkage), and nucleoside analogs, as well as conjugates or ligands. As used herein, the term “siNA duplex” or “siRNA duplex” refers to a double-stranded (“ds”) siRNA or “dsRNA” or “ds-NA” having a sense strand and an antisense strand.

As used herein with, the term “backbone” refers to the polymeric sugar-backbone of naturally occurring nucleic acids, as well as to modified counterparts and mimics thereof, to which are covalently attached the nucleobases defining a base sequence of a particular nucleic acid molecule. In some embodiments, the backbone comprises phosphodiester internucleoside linkages (in which case it is referred to as “phosphodiester backbone”). In some embodiments, in addition to phosphodiester internucleoside linkages, the backbone comprises one or more non-phosphodiester internucleoside linkages (such as, for example, phosphorothioate internucleoside linkages), as described herein. In some embodiments, the phosphodiester internucleoside linkage connects the 3′ position of a sugar moiety (e.g., ribose) of the preceding nucleoside to the 5′ position of a sugar moiety of the subsequent nucleoside (a 3′-5′ phosphodiester linkage). In some embodiments, the phosphodiester internucleoside linkage connects the 2′ position of a sugar moiety (e.g., ribose) of the preceding nucleoside to the 5′ position a sugar moiety of the subsequent nucleoside (a 2′-5′ phosphodiester linkage). Likewise, the non-phosphodiester internucleoside linkages (e.g., phosphorothioate internucleoside linkages) may connect the 3′ position of a sugar moiety (e.g., ribose) of the preceding nucleoside to the 5′ position a sugar moiety of the subsequent nucleoside (a 3′-5′ phosphorothioate linkage) or the 2′ position of a sugar moiety (e.g., ribose) of the preceding nucleoside to the 5′ position a sugar moiety of the subsequent nucleoside (a 2′-5′ phosphorothioate linkage). In some embodiments, siRNAs comprise exclusively 3′-5′ internucleoside linkages. In some embodiments, siRNAs comprise exclusively 2′-5′ internucleoside linkages. In some embodiments, siRNAs comprise a mixture of 3′-5′ internucleoside linkages and 2′-5′ internucleoside linkages.

As used herein, the term “antisense strand” or “guide strand” refers to the strand of a siRNA molecule which includes a region that is substantially complementary to a target sequence, e.g., a HSD17B13 mRNA.

As used herein, the term “sense strand” or “passenger strand” refers to the strand of a siRNA molecule that includes a region that is substantially complementary to a region of the antisense strand as that term is defined herein.

As used herein, the term “modified nucleotide” refers to a nucleotide having, independently, modifications at the sugar, nucleobase, and/or phosphodiester backbone (internucleoside linkage), and nucleoside analogs. Thus, the term modified nucleotide encompasses substitutions, additions, or removal of, e.g., a functional group or atom, to internucleoside linkages, sugar moieties, or nucleobases. The modifications suitable for use in the siRNAs of the disclosure include all types of modifications disclosed herein or known in the art. Any such modifications, as used in a siNA or siRNA molecule, are encompassed by “siRNA” and “siRNA molecule” and “siRNA duplex” and “siNA” and “siNA molecule” and “siNA duplex” for the purposes of this specification and claims. It will also be understood that the term “nucleotide” can also refer to a modified nucleotide, as further detailed herein.

As used herein, the term “nucleobase” refers to naturally-occurring nucleobases and their analogues. Examples of naturally-occurring nucleobases or their analogues include, but are not limited to, thymine, uracil, adenine, cytosine, guanine, aryl, heteroaryl, and an analogue or derivative thereof.

As used herein, the term “nucleotide overhang” or “overhang” refers to at least one unpaired nucleotide that protrudes from the duplex structure of a double-stranded RNA (e.g., siRNA duplex or dsRNA). For example, when a 3′ end of one strand of a dsRNA extends beyond the 5′ end of the other strand, or vice versa, there is a nucleotide overhang. The overhang(s) can be on the sense strand, the antisense strand or any combination thereof. Furthermore, the nucleotide(s) of an overhang can be present on the 5′ end, 3′ end or both ends of an antisense and/or sense strand of a dsRNA and can comprise modified nucleotides. Generally, if any nucleotide overhangs, as defined herein, are present, the sequence of such overhangs is not considered in determining the degree of complementarity between two sequences and such overhangs shall not be regarded as mismatches with regard to the determination of complementarity. By way of example, a sense strand of 21 nucleotides in length and an antisense strand of 21 nucleotides in length that hybridizes to form a 19 base pair duplex region with a 2 nucleotide overhang at the 3′ end of each strand would be considered to be fully complementary as the term is used herein.

As used herein, the term “blunt end” refers to an end of a dsRNA with no unpaired nucleotides, i.e., no nucleotide overhang. In some embodiments, a blunt end can be present on one or both ends of a dsRNA.

The terms “complementary,” “fully complementary” and “substantially complementary” herein can be used with respect to the base pairing between the sense strand and the antisense strand of a duplex siRNA or dsRNA, or between the antisense strand of a siRNA and a target sequence, as will be understood from the context of their use. As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of ordinary skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. In some embodiments, a sequence is “substantially complementary” to a target sequence if the sequence is at least about 80%, 85%, 90%, 95%, 96%, 97%, 98%, 99% or 100% complementary to a target sequence. Percent complementarity can be calculated, for example, by dividing the number of bases in a first sequence that are complementary to bases at corresponding positions in a second or target sequence by the total length of the first sequence. Such calculations are well within the ability of those ordinarily skilled in the art. A sequence may also be said to be substantially complementary to another sequence if there are no more than 5, 4, 3, 2, or 1 mismatches over a 30 base pair duplex region, for example, when the two sequences are hybridized. “Complementary” sequences, as used herein, can also include, or be formed entirely from, non-Watson-Crick base pairs and/or base pairs formed from modified nucleotides, in so far as the above requirements with respect to their ability to hybridize are fulfilled.

The use of percent identity (i.e., “identical”) is a common way of defining the number of differences in the nucleobases between two nucleic acid sequences. For example, where a first sequence is ACGT, a second sequence of ACGA would be considered a “non-identical” sequence with one difference. Percent identity may be calculated over the entire length of a sequence, or over a portion of the sequence. Percent identity may be calculated according to the number of nucleobases that have identical base pairing corresponding to the sequence to which it is being compared. The non-identical nucleobases may be adjacent to each other, dispersed throughout the sequence, or both. Such calculations are well within the ability of those ordinarily skilled in the art.

As used herein, “missense mutation” refers to when a change in a single base pair results in a substitution of a different amino acid in the resulting protein.

As used herein, the term “effective amount” or “therapeutically effective amount” refers to the amount of a siRNA of the present disclosure sufficient to effect beneficial or desired results, such as for example, the amount that will elicit the biological or medical response of a tissue, system, animal, or human that is being sought by a researcher, veterinarian, medical doctor, or other clinician. A therapeutically effective amount can be administered in one or more administrations, applications, or dosages and is not intended to be limited to a particular formulation or administration route. In some embodiments, “therapeutically effective amount” means an amount that alleviates at least one clinical symptom in a human patient, e.g., at least one symptom of a HSD17B13-associated disease or a liver disease.

As used herein, the terms “patient” and “subject” refer to organisms who use the siRNA molecules of the disclosure for the prevention or treatment of a medical condition, including in the methods of the present disclosure. Such organisms are preferably mammals, and more preferably humans. As used herein, a subject “in need” of treatment of an existing condition or of prophylactic treatment encompasses both a determination of need by a medical professional as well as the desire of a patient for such treatment. Administering of the compound (e.g., a siNA or siRNA of the present disclosure) to the subject includes both self-administration and administration to the patient by another.

As used herein, the term “active agent” or “active ingredient” or “therapeutic agent” refers to an ingredient with a pharmacological effect, such as a therapeutic effect, at a relevant dose. This includes siRNA molecules according to the disclosure.

As used herein, a “liver disease treatment agent” is an active agent which can be used to treat liver disease, either alone or in combination with another active agent, and is other than the siRNA of the present disclosure.

As used herein, the term “pharmaceutical composition” refers to the combination of at least one active agent with a carrier, inert or active, making the composition especially suitable for diagnostic or therapeutic use in vivo or ex vivo. In some embodiments, the term “pharmaceutical composition” means a composition comprising a siRNA molecule as described herein and at least one additional component selected from pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the mode of administration and dosage form used.

As used herein, the term “pharmaceutically acceptable carrier” refers to any pharmaceutical carrier, diluent, adjuvant, excipient, or vehicle, including those described herein, for example, solvents, buffers, solutions (e.g., a phosphate buffered saline solution), water, emulsions (e.g., such as an oil/water or water/oil emulsions), various types of wetting agents, stabilizers, preservatives, antibacterial and antifungal agents, dispersion media, coatings, isotonic and absorption delaying agents and the like acceptable for use in formulating pharmaceuticals, including, for example, pharmaceuticals suitable for administration to humans. For examples of carriers, see, for example, Martin, Remington's Pharmaceutical Sciences, 15th Ed., Mack Publ. Co., Easton, PA [1975].

As used herein, the terms “treat”, “treating”, and “treatment” include any effect, e.g., lessening, reducing, modulating, ameliorating or eliminating, that results in the improvement of the condition, disease, disorder, and the like; or of one or more symptoms associated with the condition, disease, or disorder; or of the cause(s) of the condition, disease, or disorder. For example, with respect to HSD17B13-associated disease, the terms “treat”, “treating”, and “treatment” include, but are not limited to, alleviation or amelioration of one or more symptoms associated with HSD17B13 gene expression and/or HSD17B13 protein production, e.g., fatty liver (steatosis), nonalcoholic steatohepatitis (NASH), cirrhosis of the liver, accumulation of fat in the liver, inflammation of the liver, hepatocellular necrosis, liver fibrosis, obesity, hepatocellular carcinoma (HCC) or nonalcoholic fatty liver disease (NAFLD). “Treatment” can also mean prolonging survival as compared to expected survival in the absence of treatment.

As used herein, the terms “alleviate” and “alleviating” refer to reducing the severity of the condition and/or a symptom thereof, such as reducing the severity by, for example, at least 10%, 20%, 30%, 40%, 50%, 60%, 70%, 80%, 90%, or 95%.

As used herein, the term “downregulate” or “downregulating” is used interchangeably with “reducing”, “inhibiting”, or “suppressing” or other similar terms, and includes any level of downregulation.

As used herein, the term “HSD17B13 gene” refers to the hydroxysteroid 17-beta dehydrogenase 13 gene and includes variants thereof. HSD17B13 has a sequence shown in the nucleotide sequence of SEQ ID NO: 261, which corresponds to the nucleotide sequence of the coding sequence of GenBank Accession No. NM_178135.5 (nucleotides 42 to 944), which is incorporated by reference in its entirety. Additional examples of HSD17B13 gene sequences, including for other mammalian genes, are readily available using public databases, including, for example, NCBI RefSeq, GenBank, UniProt, and OMIM.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

siRNA Molecules

Disclosed herein are double-stranded short (or small) interfering RNA (siRNA) molecules that specifically downregulate expression of a hydroxysteroid 17-beta dehydrogenase 13 (HDS17B13) gene.

In some embodiments, the double-stranded siRNA molecule comprises (a) a sense strand comprising a nucleotide sequence that is at least about 60%, 65%, 70%, 75%, 80%, 85%, 90%, 95%, or 100% identical to the nucleotide sequence of any one of SEQ ID NOs: 1-100, 201-230, 262-287, 314-445, 576-603 or 638; and/or (b) an antisense strand comprising a nucleotide sequence that is at least about 60%, 65%, 70%, 75%, 80%, 85%, 90%, 95%, or 100% identical to the nucleotide sequence of any one of SEQ ID NOs: 101-200, 231-260, 288-313, 446-575, 604-637 or 639-644.

In some embodiments, the double-stranded siRNA molecule comprises (a) a sense strand comprising a nucleotide sequence of any one SEQ ID NOs: 1-100, 201-230, 262-287, 314-445, 576-603 or 638; and/or (b) an antisense strand comprising a nucleotide sequence of any one of SEQ ID NOs: 101-200, 231-260, 288-313, 446-575, 604-637 or 639-644.

In some embodiments, the double-stranded siRNA molecule comprises a sense strand comprising a nucleotide sequence of any one of SEQ ID NOs: 1-100 or 201-230. In some embodiments, the siRNA molecule comprises an antisense strand comprising a nucleotide sequence of any one of SEQ ID NOs: 101-200 or 231-260. In some embodiments, the siRNA molecule comprises (a) a sense strand comprising a nucleotide sequence of any one of SEQ ID NOs: 1-100 or 201-230 and (b) an antisense strand comprising a nucleotide sequence of any one of SEQ ID NOs: 101-200 or 231-260.

In some embodiments, the double-stranded siRNA molecule comprises a sense strand comprising a nucleotide sequence of any one of SEQ ID NOs: 316-445. In some embodiments, the siRNA molecule comprises an antisense strand comprising a nucleotide sequence of any one of SEQ ID NOs: 446-575. In some embodiments, the siRNA molecule comprises (a) a sense strand comprising a nucleotide sequence of any one of SEQ ID NOs: 316-445 and (b) an antisense strand comprising a nucleotide sequence of any one of SEQ ID NOs: 446-575.

In some embodiments, the double-stranded siRNA molecule comprises a sense strand comprising a nucleotide sequence of any one of SEQ ID NOs: 262-287, 314 or 315. In some embodiments, the siRNA molecule comprises an antisense strand comprising a nucleotide sequence of any one of SEQ ID NOs: 288-313. In some embodiments, the siRNA molecule comprises (a) a sense strand comprising a nucleotide sequence of any one of SEQ ID NOs: 262-287, 314 or 315 and (b) an antisense strand comprising a nucleotide sequence of any one of SEQ ID NOs: 288-313.

In some embodiments, the double-stranded siRNA molecule comprises a sense strand comprising a nucleotide sequence of any one of SEQ ID NOs: 576-603 or 638. In some embodiments, the siRNA molecule comprises an antisense strand comprising a nucleotide sequence of any one of SEQ ID NOs: 604-637 or 639-644. In some embodiments, the siRNA molecule comprises (a) a sense strand comprising a nucleotide sequence of any one of SEQ ID NOs: 576-603 or 638 and (b) an antisense strand comprising a nucleotide sequence of any one of SEQ ID NOs: 604-637 or 639-644.

In some embodiments, the double-stranded siRNA molecule comprises (a) a sense strand comprising at least about 15, 16, 17, 18, 19, 20, or 21 consecutive nucleotides of the nucleotide sequence of any one SEQ ID NOs: 1-100, 201-230, 262-287, 314-445, 576-603 or 638; and/or (b) an antisense strand comprising at least about 15, 16, 17, 18, 19, 20, 21, 22, or 23 consecutive nucleotides of the nucleotide sequence of any one of SEQ ID NOs: 101-200, 231-260, 288-313, 446-575, 604-637 or 639-644

In some embodiments, the double-stranded siRNA molecule comprises (a) a sense strand comprising a nucleotide sequence having at least about 15, 16, 17, 18, 19, 20, or 21 consecutive nucleotides of the nucleotide sequence of any one of SEQ ID NOs: 1-100, 201-230, 262-287, 314-445, 576-603 or 638; and/or (b) an antisense strand comprising a nucleotide sequence having at least about 15, 16, 17, 18, 19, 20, 21, 22, or 23 consecutive nucleotides of the nucleotide sequence of any one of SEQ ID NOs: 101-200, 231-260, 288-313, 446-575, 604-637 or 639-644.

In some embodiments, at least one end of the double-stranded siRNA molecule is a blunt end. In some embodiments, both ends of the double-stranded siRNA molecule are blunt ends. In some embodiments, one end of the double-stranded siRNA molecule comprises a blunt end and one end of the double-stranded siRNA molecule comprises an overhang.

In some embodiments, at least one end of the siRNA molecule comprises an overhang, wherein the overhang comprises at least one unpaired nucleotide. In some embodiments, at least one end of the siRNA molecule comprises an overhang, wherein the overhang comprises at least two unpaired nucleotides. In some embodiments, both ends of the siRNA molecule comprise an overhang, wherein the overhang comprises at least one unpaired nucleotide. In some embodiments, both ends of the siRNA molecule comprise an overhang, wherein the overhang comprises at least two unpaired nucleotides. In some embodiments, the siRNA molecule comprises an overhang of two unpaired nucleotides at the 3′ end of the sense strand. In some embodiments, the siRNA molecule comprises an overhang of two unpaired nucleotides at the 3′ end of the antisense strand. In some embodiments, the siRNA molecule comprises an overhang of two unpaired nucleotides at the 3′ end of the sense strand and the 3′ end of the antisense strand.

In some embodiments, the double stranded siRNA molecule is selected from any one of siNA Duplex ID Nos. ds-siNA D1-D178 or mds-siNA MD1-MD178. In some embodiments, the double stranded siRNA molecule is selected from any one of siRNA Duplex ID Nos. ds-siNA D1-D178. In some embodiments, the double stranded siRNA molecule is selected from any one of siRNA Duplex ID Nos. mds-siNA MD1-MD178.

In some embodiments, the double stranded siRNA molecule is selected from any one of the siRNA Duplexes of Table 8 or Table 9 or Table 10 or Table 11 or Table 12. In some embodiments, the double stranded siRNA molecule is selected from any one of the siRNA Duplexes of Table 8. In some embodiments, the double stranded siRNA molecule is selected from any one of the siRNA Duplexes of Table 9. In some embodiments, the double stranded siRNA molecule is selected from any one of the siRNA Duplexes of Table 10. In some embodiments, the double stranded siRNA molecule is selected from any one of the siRNA Duplexes of Table 11. In some embodiments, the double stranded siRNA molecule is selected from any one of the siRNA Duplexes of Table 12.

In some embodiments, the double stranded siRNA molecule is about 17 to about 29 base pairs in length, or from 19-23 base pairs, or from 19-21 base pairs, one strand of which is complementary to a target mRNA, that when added to a cell having the target mRNA, or produced in the cell in vivo, causes degradation of the target mRNA.

In some embodiments, the siRNA molecules of the disclosure comprise a nucleotide sequence that is complementary to a nucleotide sequence of a target gene. In some embodiments, the siRNA molecule of the disclosure interacts with a nucleotide sequence of a target gene in a manner that causes inhibition of expression of the target gene.

The siRNA molecules can be obtained using any one of a number of techniques known to those of ordinary skill in the art. In some embodiments, the siRNA molecules may be synthesized as two separate, complementary nucleic acid molecules, or as a single nucleic acid molecule with two complementary regions. For example, the siRNAs of the disclosure may be chemically synthesized using appropriately protected ribonucleoside phosphoramidites and a conventional RNA synthesizer or other well-known methods. In addition, the siRNAs may be produced by a commercial supplier, such as, for example, Dharmacon/Horizon (Lafayette, Colo., USA), Glen Research (Sterling, Va., USA), ChemGenes (Ashland, Mass., USA) and Cruachem (Glasgow, UK). In some embodiments, the siRNA molecules may be encoded by a plasmid.

### Sense Strand

Any of the siRNA molecules described herein may comprise a sense strand. In some embodiments, the sense strand comprises between about 15 to about 50 nucleotides. In some embodiments, the sense strand comprises between about 15 to about 45 nucleotides. In some embodiments, the sense strand comprises between about 15 to about 40 nucleotides. In some embodiments, the sense strand comprises between about 15 to about 35 nucleotides. In some embodiments, the sense strand comprises between about 15 to about 30 nucleotides. In some embodiments, the sense strand comprises between about 15 to about 25 nucleotides. In some embodiments, the sense strand comprises between about 17 to about 23 nucleotides. In some embodiments, the sense strand comprises between about 17 to about 22 nucleotides. In some embodiments, the sense strand comprises between about 17 to about 21 nucleotides. In some embodiments, the sense strand comprises between about 18 to about 23 nucleotides. In some embodiments, the sense strand comprises between about 18 to about 22 nucleotides. In some embodiments, the sense strand comprises between about 18 to about 21 nucleotides. In some embodiments, the sense strand comprises between about 19 to about 23 nucleotides. In some embodiments, the sense strand comprises between about 19 to about 22 nucleotides. In some embodiments, the sense strand comprises between about 19 to about 21 nucleotides.

In some embodiments, the sense strand comprises at least about 15, 16, 17, 18, 19, 20, 21, 22, 23, 24, 25, 26, 27, 28, 29, or 30 or more nucleotides. In some embodiments, the sense strand comprises at least about 15 nucleotides. In some embodiments, the sense strand comprises at least about 16 nucleotides. In some embodiments, the sense strand comprises at least about 17 nucleotides. In some embodiments, the sense strand comprises at least about 18 nucleotides. In some embodiments, the sense strand comprises at least about 19 nucleotides. In some embodiments, the sense strand comprises at least about 20 nucleotides. In some embodiments, the sense strand comprises at least about 21 nucleotides. In some embodiments, the sense strand comprises at least about 22 nucleotides. In some embodiments, the sense strand comprises at least about 23 nucleotides.

In some embodiments, the sense strand comprises less than about 50, 45, 40, 35, 30, 29, 28, 27, 26, 25, 24, 23, 22, 21, 20, or 19 or fewer nucleotides. In some embodiments, the sense strand comprises less than about 30 nucleotides. In some embodiments, the sense strand comprises less than about 25 nucleotides. In some embodiments, the sense strand comprises less than about 24 nucleotides. In some embodiments, the sense strand comprises less than about 23 nucleotides. In some embodiments, the sense strand comprises less than about 22 nucleotides. In some embodiments, the sense strand comprises less than about 21 nucleotides. In some embodiments, the sense strand comprises less than about 20 nucleotides. In some embodiments, the sense strand comprises less than about 19 nucleotides.

In some embodiments, the sense strand comprises a sequence that is at least about 60%, 65%, 70%, 75%, 80%, 85%, 90%, 95%, or 100% identical to a fragment of the HSD17B13 gene across the entire length of the sense strand. In some embodiments, the sense strand comprises a sequence that is at least about 70% identical to a fragment of the HSD17B13 gene across the entire length of the sense strand. In some embodiments, the sense strand comprises a sequence that is at least about 75% identical to a fragment of the HSD17B13 gene across the entire length of the sense strand. In some embodiments, the sense strand comprises a sequence that is at least about 80% identical to a fragment of the HSD17B13 gene across the entire length of the sense strand. In some embodiments, the sense strand comprises a sequence that is at least about 85% identical to a fragment of the HSD17B13 gene across the entire length of the sense strand. In some embodiments, the sense strand comprises a sequence that is at least about 90% identical to a fragment of the HSD17B13 gene across the entire length of the sense strand. In some embodiments, the sense strand comprises a sequence that is at least about 95% identical to a fragment of the HSD17B13 gene across the entire length of the sense strand. In some embodiments, the sense strand comprises a sequence that is about 100% identical to a fragment of the HSD17B13 gene across the entire length of the sense strand. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the sense strand comprises a sequence having between about 15 to about 50 consecutive nucleotides of a fragment of the HSD17B13 gene. In some embodiments, the sense strand comprises a sequence having between about 15 to about 45 consecutive nucleotides of a fragment of the HSD17B13 gene. In some embodiments, the sense strand comprises a sequence having between about 15 to about 40 consecutive nucleotides of a fragment of the HSD17B13 gene. In some embodiments, the sense strand comprises a sequence having between about 15 to about 35 consecutive nucleotides of a fragment of the HSD17B13 gene. In some embodiments, the sense strand comprises a sequence having between about 15 to about 30 consecutive nucleotides of a fragment of the HSD17B13 gene. In some embodiments, the sense strand comprises a sequence having between about 15 to about 25 consecutive nucleotides of a fragment of the HSD17B13 gene. In some embodiments, the sense strand comprises between about 17 to about 23 consecutive nucleotides of a fragment of the HSD17B13 gene. In some embodiments, the sense strand comprises between about 17 to about 22 consecutive nucleotides of a fragment of the HSD17B13 gene. In some embodiments, the sense strand comprises between about 17 to about 21 consecutive nucleotides of a fragment of the HSD17B13 gene. In some embodiments, the sense strand comprises between about 18 to about 23 consecutive nucleotides of a fragment of the HSD17B13 gene. In some embodiments, the sense strand comprises between about 18 to about 22 consecutive nucleotides of a fragment of the HSD17B13 gene. In some embodiments, the sense strand comprises between about 18 to about 21 consecutive nucleotides of a fragment of the HSD17B13 gene. In some embodiments, the sense strand comprises between about 19 to about 23 consecutive nucleotides of a fragment of the HSD17B13 gene. In some embodiments, the sense strand comprises between about 19 to about 22 consecutive nucleotides of a fragment of the HSD17B13 gene. In some embodiments, the sense strand comprises between about 19 to about 21 consecutive nucleotides of a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the sense strand comprises a sequence having at least about 15, 16, 17, 18, 19, 20, 21, 22, 23, 24, 25, 26, 27, 28, 29, or 30 or more consecutive nucleotides of a fragment of the HSD17B13 gene. In some embodiments, the sense strand comprises a sequence having at least about 15 consecutive nucleotides of a fragment of the HSD17B13 gene. In some embodiments, the sense strand comprises a sequence having at least about 16 consecutive nucleotides of a fragment of the HSD17B13 gene. In some embodiments, the sense strand comprises a sequence having at least about 17 consecutive nucleotides of a fragment of the HSD17B13 gene. In some embodiments, the sense strand comprises a sequence having at least about 18 consecutive nucleotides of a fragment of the HSD17B13 gene. In some embodiments, the sense strand comprises a sequence having at least about 19 consecutive nucleotides of a fragment of the HSD17B13 gene. In some embodiments, the sense strand comprises a sequence having at least about 20 consecutive nucleotides of a fragment of the HSD17B13 gene. In some embodiments, the sense strand comprises a sequence having at least about 21 consecutive nucleotides of a fragment of the HSD17B13 gene. In some embodiments, the sense strand comprises a sequence having at least about 22 consecutive nucleotides of a fragment of the HSD17B13 gene. In some embodiments, the sense strand comprises a sequence having at least about 23 consecutive nucleotides of a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the sense strand comprises a sequence having less than about 50, 45, 40, 35, 30, 29, 28, 27, 26, 25, 24, 23, 22, 21, 20, or 19 or fewer consecutive nucleotides of a fragment of the HSD17B13 gene. In some embodiments, the sense strand comprises a sequence having less than about 35 consecutive nucleotides of a fragment of the HSD17B13 gene. In some embodiments, the sense strand comprises a sequence having less than about 30 consecutive nucleotides of a fragment of the HSD17B13 gene. In some embodiments, the sense strand comprises a sequence having less than about 25 consecutive nucleotides of a fragment of the HSD17B13 gene. In some embodiments, the sense strand comprises a sequence having less than about 24 consecutive nucleotides of a fragment of the HSD17B13 gene. In some embodiments, the sense strand comprises a sequence having less than about 23 consecutive nucleotides of a fragment of the HSD17B13 gene. In some embodiments, the sense strand comprises a sequence having less than about 22 consecutive nucleotides of a fragment of the HSD17B13 gene. In some embodiments, the sense strand comprises a sequence having less than about 21 consecutive nucleotides of a fragment of the HSD17B13 gene. In some embodiments, the sense strand comprises a sequence having less than about 20 consecutive nucleotides of a fragment of the HSD17B13 gene. In some embodiments, the sense strand comprises a sequence having less than about 19 consecutive nucleotides of a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the sense strand comprises a sequence having less than or equal to 5, 4, 3, 2, or 1 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having less than or equal to 5 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having less than or equal to 4 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having less than or equal to 3 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having less than or equal to 2 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having less than or equal to 1 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the sense strand comprises a sequence having 0 nucleobase differences to a fragment of the HSD17B13 gene across the entire length of the sense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the sense strand comprises a nucleotide sequence of any one of SEQ ID NOs: 1-100, 201-230, 262-287, 314-445, 576-603 or 638. In some embodiments, the sense strand comprises a nucleotide sequence that is at least about 60%, 65%, 70%, 75%, 80%, 85%, 90%, 95%, or 100% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 70% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 75% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 80% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 85% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 90% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is at least about 95% identical to the nucleotide sequence of any one of SEQ ID NOs: 1-100, 201-230, 262-287, 314-445, 576-603 or 638 across the entire length of sense strand. In some embodiments, the sense strand comprises a nucleotide sequence that is about 100% identical to the nucleotide sequence of any one of SEQ ID NOs: 1-100, 201-230, 262-287, 314-445, 576-603 or 638 across the entire length of sense strand.

In some embodiments, the sense strand comprises at least about 15, 16, 17, 18, 19, 20, or 21 consecutive nucleotides of the nucleotide sequence of any one of SEQ ID NOs: 1-100, 201-230, 262-287, 314-445, 576-603 or 638. In some embodiments, the sense strand comprises at least about 17 consecutive nucleotides of the nucleotide sequence of any one of SEQ ID NOs: 1-100, 201-230, 262-287, 314-445, 576-603 or 638. In some embodiments, the sense strand comprises at least about 18 consecutive nucleotides of the nucleotide sequence of any one of SEQ ID NOs: 1-100, 201-230, 262-287, 314-445, 576-603 or 638. In some embodiments, the sense strand comprises at least about 19 consecutive nucleotides of the nucleotide sequence of any one of SEQ ID NOs: 1-100, 201-230, 262-287, 314-445, 576-603 or 638. In some embodiments, the sense strand comprises at least about 20 consecutive nucleotides of the nucleotide sequence of any one of SEQ ID NOs: 1-100, 201-230, 262-287, 314-445, 576-603 or 638. In some embodiments, the sense strand comprises at least about 21 consecutive nucleotides of the nucleotide sequence of any one of SEQ ID NOs: 1-100, 201-230, 262-287, 314-445, 576-603 or 638.

In some embodiments, the sense strand comprises a nucleotide sequence having less than or equal to 5, 4, 3, 2, or 1 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having less than or equal to 5 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having less than or equal to 4 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having less than or equal to 3 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having less than or equal to 2 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having less than or equal to 1 mismatches to the nucleotide sequence of any one of SEQ ID NOs: 101-200, 231-260, 288-313, 446-575, 604-637 or 639-644 across the entire length of the sense strand. In some embodiments, the sense strand comprises a nucleotide sequence having 0 mismatches to the nucleotide sequence of any one of SEQ ID NOs: 101-200, 231-260, 288-313, 446-575, 604-637 or 639-644 across the entire length of the sense strand.

In some embodiments, the sense strand comprises a nucleotide sequence of any of the sense strands listed in Table 8 or Table 9 or Table 10 or Table 11 or Table 12. In some embodiments, the sense strand comprises a nucleotide sequence of any of the sense strands listed in Table 8. In some embodiments, the sense strand comprises a nucleotide sequence of any of the sense strands listed in Table 9. In some embodiments, the sense strand comprises a nucleotide sequence of any of the sense strands listed in Table 10. In some embodiments, the sense strand comprises a nucleotide sequence of any of the sense strands listed in Table 11. In some embodiments, the sense strand comprises a nucleotide sequence of any of the sense strands listed in Table 12.

In some embodiments, the sense strand may comprise an overhang sequence. In some embodiments, the overhang sequence comprises at least about 1, 2, 3, 4, or 5 or more nucleotides. In some embodiments, the overhang sequence comprises at least about 1 nucleotide. In some embodiments, the overhang sequence comprises at least about 2 nucleotides. In some embodiments, the overhang sequence comprises at least about 3 nucleotides. In some embodiments, the overhang sequence comprises at least about 4 nucleotides. In some embodiments, the overhang sequence comprises at least about 5 nucleotides.

In some embodiments, the sense strand may comprise at least 1, 2, 3, or 4 phosphorothioate internucleoside linkages. In some embodiments, at least one phosphorothioate internucleoside linkage is between the nucleotides at positions 1 and 2 from the 5′ end of the sense strand. In some embodiments, at least one phosphorothioate internucleoside linkage is between the nucleotides at positions 2 and 3 from the 5′ end of the sense strand. In some embodiments, at least one phosphorothioate internucleoside linkage is between the nucleotides at positions 1 and 2 from the 3′ end of the sense strand. In some embodiments, at least one phosphorothioate internucleoside linkage is between the nucleotides at positions 2 and 3 from the 3′ end of the sense strand.

In some embodiments, the sense strand may comprise a nucleotide sequence comprising 2′-fluoro nucleotides at positions 3, 7-9, 12 and 17. In some embodiments, the sense strand may comprise a nucleotide sequence comprising 2′-fluoro nucleotides at positions 3, 7, 8, and 17. In some embodiments, the sense strand may comprise a nucleotide sequence comprising 2′-fluoro nucleotides at positions 5 and 7-9 from the 5′ end of the nucleotide sequence. In some embodiments, the sense strand may comprise a nucleotide sequence comprising 2′-fluoro nucleotides at positions 7 and 9-11 from the 5′ end of the nucleotide sequence. In some embodiments, the sense strand may comprise a nucleotide comprising 2′-fluoro nucleotides at positions 5, 9-11, 14, and 19 from the 5′ end of the nucleotide sequence. In some embodiments, the sense strand may comprise a nucleotide sequence consisting of 19 to 23, or 19 to 21, nucleotides, wherein 2′-fluoro nucleotides are at positions 5 and 7-9 from the 5′ end of the nucleotide sequence. In some embodiments, the sense strand may comprise a nucleotide sequence consisting of 19 to 23, or 19 to 21, nucleotides, wherein 2′-fluoro nucleotides are at positions 7 and 9-11 from the 5′ end of the nucleotide sequence. In some embodiments, the sense strand may comprise a nucleotide sequence consisting of 19 to 23, or 19 to 21, nucleotides, wherein 2′-fluoro nucleotides are at positions 5, 9-11, 14, and 19 from the 5′ end of the nucleotide sequence. In some embodiments, the nucleotide at position 5, 9, 10, and/or 11 from the 5′ end of the first nucleotide sequence is a 2′-fluoro nucleotide. Antisense Strand

Any of the siRNA molecules described herein may comprise an antisense strand. In some embodiments, the antisense strand comprises between about 15 to about 50 nucleotides. In some embodiments, the antisense strand comprises between about 15 to about 45 nucleotides. In some embodiments, the antisense strand comprises between about 15 to about 40 nucleotides. In some embodiments, the antisense strand comprises between about 15 to about 35 nucleotides. In some embodiments, the antisense strand comprises between about 15 to about 30 nucleotides. In some embodiments, the antisense strand comprises between about 15 to about 25 nucleotides. In some embodiments, the antisense strand comprises between about 17 to about 23 nucleotides. In some embodiments, the antisense strand comprises between about 17 to about 22 nucleotides. In some embodiments, the antisense strand comprises between about 17 to about 21 nucleotides. In some embodiments, the antisense strand comprises between about 18 to about 23 nucleotides. In some embodiments, the antisense strand comprises between about 18 to about 22 nucleotides. In some embodiments, the antisense strand comprises between about 18 to about 21 nucleotides. In some embodiments, the antisense strand comprises between about 19 to about 23 nucleotides. In some embodiments, the antisense strand comprises between about 19 to about 22 nucleotides. In some embodiments, the antisense strand comprises between about 19 to about 21 nucleotides.

In some embodiments, the antisense strand comprises at least about 15, 16, 17, 18, 19, 20, 21, 22, 23, 24, 25, 26, 27, 28, 29, or 30 or more nucleotides. In some embodiments, the antisense strand comprises at least about 15 nucleotides. In some embodiments, the antisense strand comprises at least about 16 nucleotides. In some embodiments, the antisense strand comprises at least about 17 nucleotides. In some embodiments, the antisense strand comprises at least about 18 nucleotides. In some embodiments, the antisense strand comprises at least about 19 nucleotides. In some embodiments, the antisense strand comprises at least about 20 nucleotides. In some embodiments, the antisense strand comprises at least about 21 nucleotides. In some embodiments, the antisense strand comprises at least about 22 nucleotides. In some embodiments, the antisense strand comprises at least about 23 nucleotides.

In some embodiments, the antisense strand comprises less than about 50, 45, 40, 35, 30, 29, 28, 27, 26, 25, 24, 23, 22, 21, 20, or 19 or fewer nucleotides. In some embodiments, the antisense strand comprises less than about 30 nucleotides. In some embodiments, the antisense strand comprises less than about 25 nucleotides. In some embodiments, the antisense strand comprises less than about 24 nucleotides. In some embodiments, the antisense strand comprises less than about 23 nucleotides. In some embodiments, the antisense strand comprises less than about 22 nucleotides. In some embodiments, the antisense strand comprises less than about 21 nucleotides. In some embodiments, the antisense strand comprises less than about 20 nucleotides. In some embodiments, the antisense strand comprises less than about 19 nucleotides.

In some embodiments, the antisense strand comprises a sequence that is at least about 60%, 65%, 70%, 75%, 80%, 85%, 90%, 95%, or 100% complementary to a fragment of the HSD17B13 gene across the entire length of the antisense strand. In some embodiments, the antisense strand comprises a sequence that is at least about 70% complementary to a fragment of the HSD17B13 gene across the entire length of the antisense strand. In some embodiments, the antisense strand comprises a sequence that is at least about 75% complementary to a fragment of the HSD17B13 gene across the entire length of the antisense strand. In some embodiments, the antisense strand comprises a sequence that is at least about 80% complementary to a fragment of the HSD17B13 gene across the entire length of the antisense strand. In some embodiments, the antisense strand comprises a sequence that is at least about 85% complementary to a fragment of the HSD17B13 gene across the entire length of the antisense strand. In some embodiments, the antisense strand comprises a sequence that is at least about 90% complementary to a fragment of the HSD17B13 gene across the entire length of the antisense strand. In some embodiments, the antisense strand comprises a sequence that is at least about 95% complementary to a fragment of the HSD17B13 gene across the entire length of the antisense strand. In some embodiments, the antisense strand comprises a sequence that is about 100% complementary to a fragment of the HSD17B13 gene across the entire length of the antisense strand. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the antisense strand comprises a sequence having between about 15 to about 50 consecutive nucleotides complementary to a fragment of the HSD17B13 gene. In some embodiments, the antisense strand comprises a sequence having between about 15 to about 45 consecutive nucleotides complementary to a fragment of the HSD17B13 gene. In some embodiments, the antisense strand comprises a sequence having between about 15 to about 40 consecutive nucleotides complementary to a fragment of the HSD17B13 gene. In some embodiments, the antisense strand comprises a sequence having between about 15 to about 35 consecutive nucleotides complementary to a fragment of the HSD17B13 gene. In some embodiments, the antisense strand comprises a sequence having between about 15 to about 30 consecutive nucleotides complementary to a fragment of the HSD17B13 gene. In some embodiments, the antisense strand comprises a sequence having between about 15 to about 25 consecutive nucleotides complementary to a fragment of the HSD17B13 gene. In some embodiments, the antisense strand comprises between about 17 to about 23 consecutive nucleotides complementary to a fragment of the HSD17B13 gene. In some embodiments, the antisense strand comprises between about 17 to about 22 consecutive nucleotides complementary to a fragment of the HSD17B13 gene. In some embodiments, the antisense strand comprises between about 17 to about 21 consecutive nucleotides complementary to a fragment of the HSD17B13 gene. In some embodiments, the antisense strand comprises between about 18 to about 23 consecutive nucleotides complementary to a fragment of the HSD17B13 gene. In some embodiments, the antisense strand comprises between about 18 to about 22 consecutive nucleotides complementary to a fragment of the HSD17B13 gene. In some embodiments, the antisense strand comprises between about 18 to about 21 consecutive nucleotides complementary to a fragment of the HSD17B13 gene. In some embodiments, the antisense strand comprises between about 19 to about 23 consecutive nucleotides complementary to a fragment of the HSD17B13 gene. In some embodiments, the antisense strand comprises between about 19 to about 22 consecutive nucleotides of a fragment of the HSD17B13 gene. In some embodiments, the antisense strand comprises between about 19 to about 21 consecutive nucleotides complementary to a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the antisense strand comprises a sequence having at least about 15, 16, 17, 18, 19, 20, 21, 22, 23, 24, 25, 26, 27, 28, 29, or 30 or more consecutive nucleotides complementary to a fragment of the HSD17B13 gene. In some embodiments, the antisense strand comprises a sequence having at least about 15 consecutive nucleotides complementary to a fragment of the HSD17B13 gene. In some embodiments, the antisense strand comprises a sequence having at least about 16 consecutive nucleotides complementary to a fragment of the HSD17B13 gene. In some embodiments, the antisense strand comprises a sequence having at least about 17 consecutive nucleotides complementary to a fragment of the HSD17B13 gene. In some embodiments, the antisense strand comprises a sequence having at least about 18 consecutive nucleotides complementary to a fragment of the HSD17B13 gene. In some embodiments, the antisense strand comprises a sequence having at least about 19 consecutive nucleotides complementary to a fragment of the HSD17B13 gene. In some embodiments, the antisense strand comprises a sequence having at least about 20 consecutive nucleotides complementary to a fragment of the HSD17B13 gene. In some embodiments, the antisense strand comprises a sequence having at least about 21 consecutive nucleotides complementary to a fragment of the HSD17B13 gene. In some embodiments, the antisense strand comprises a sequence having at least about 22 consecutive nucleotides complementary to a fragment of the HSD17B13 gene. In some embodiments, the antisense strand comprises a sequence having at least about 23 consecutive nucleotides complementary to a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the antisense strand comprises a sequence having less than about 50, 45, 40, 35, 30, 29, 28, 27, 26, 25, 24, 23, 22, 21, 20, or 19 or fewer consecutive nucleotides complementary to a fragment of the HSD17B13 gene. In some embodiments, the antisense strand comprises a sequence having less than about 35 consecutive nucleotides complementary to a fragment of the HSD17B13 gene. In some embodiments, the antisense strand comprises a sequence having less than about 30 consecutive nucleotides complementary to a fragment of the HSD17B13 gene. In some embodiments, the antisense strand comprises a sequence having less than about 25 consecutive nucleotides complementary to a fragment of the HSD17B13 gene. In some embodiments, the antisense strand comprises a sequence having less than about 24 consecutive nucleotides complementary to a fragment of the HSD17B13 gene. In some embodiments, the antisense strand comprises a sequence having less than about 23 consecutive nucleotides complementary to a fragment of the HSD17B13 gene. In some embodiments, the antisense strand comprises a sequence having less than about 22 consecutive nucleotides complementary to a fragment of the HSD17B13 gene. In some embodiments, the antisense strand comprises a sequence having less than about 21 consecutive nucleotides complementary to a fragment of the HSD17B13 gene. In some embodiments, the antisense strand comprises a sequence having less than about 20 consecutive nucleotides complementary to a fragment of the HSD17B13 gene. In some embodiments, the antisense strand comprises a sequence having less than about 19 consecutive nucleotides complementary to a fragment of the HSD17B13 gene. In some embodiments, the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the antisense strand comprises a sequence having less than or equal to 5, 4, 3, 2, or 1 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having less than or equal to 5 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having less than or equal to 4 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having less than or equal to 3 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having less than or equal to 2 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having less than or equal to 1 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the antisense strand comprises a sequence having 0 mismatches to a fragment of the HSD17B13 gene across the entire length of the antisense strand, wherein the fragment of the HSD17B13 gene consists of about 15, 16, 17, 18, 19, 20, 21, 22, 23, 24, 25, 26, 27, 28, 29, or 30 consecutive nucleotides of the HSD17B13 gene. In some embodiments, the fragment of the HSD17B13 gene consists of about 15 consecutive nucleotides of the HSD17B13 gene. In some embodiments, the fragment of the HSD17B13 gene consists of about 16 consecutive nucleotides of the HSD17B13 gene. In some embodiments, the fragment of the HSD17B13 gene consists of about 17 consecutive nucleotides of the HSD17B13 gene. In some embodiments, the fragment of the HSD17B13 gene consists of about 18 consecutive nucleotides of the HSD17B13 gene. In some embodiments, the fragment of the HSD17B13 gene consists of about 19 consecutive nucleotides of the HSD17B13 gene. In some embodiments, the fragment of the HSD17B13 gene consists of about 20 consecutive nucleotides of the HSD17B13 gene. In some embodiments, the fragment of the HSD17B13 gene consists of about 21 consecutive nucleotides of the HSD17B13 gene. In some embodiments, the fragment of the HSD17B13 gene consists of about 22 consecutive nucleotides of the HSD17B13 gene. In some embodiments, the fragment of the HSD17B13 gene consists of about 23 consecutive nucleotides of the HSD17B13 gene.

In some embodiments, the antisense strand comprises a nucleotide sequence of any one of SEQ ID NOs: 101-200, 231-260, 288-313, 446-575, 604-637 or 639-644. In some embodiments, the antisense strand comprises a nucleotide sequence that is at least about 60%, 65%, 70%, 75%, 80%, 85%, 90%, 95%, or 100% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 70% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 75% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 80% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 85% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 90% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is at least about 95% identical to the nucleotide sequence of any one of SEQ ID NOs: 101-200, 231-260, 288-313, 446-575, 604-637 or 639-644 across the entire length of antisense strand. In some embodiments, the antisense strand comprises a nucleotide sequence that is about 100% identical to the nucleotide sequence of any one of SEQ ID NOs: 101-200, 231-260, 288-313, 446-575, 604-637 or 639-644 across the entire length of antisense strand

In some embodiments, the antisense strand comprises at least about 15, 16, 17, 18, 19, 20, 21, 22, or 23 consecutive nucleotides of the nucleotide sequence of any one of SEQ ID NOs: 101-200, 231-260, 288-313, 446-575, 604-637 or 639-644. In some embodiments, the antisense strand comprises at least about 17 consecutive nucleotides of the nucleotide sequence of any one of SEQ ID NOs: 101-200, 231-260, 288-313, 446-575, 604-637 or 639-644. In some embodiments, the antisense strand comprises at least about 18 consecutive nucleotides of the nucleotide sequence of any one of SEQ ID NOs: 101-200, 231-260, 288-313, 446-575, 604-637 or 639-644. In some embodiments, the antisense strand comprises at least about 19 consecutive nucleotides of the nucleotide sequence of any one of SEQ ID NOs: 101-200, 231-260, 288-313, 446-575, 604-637 or 639-644. In some embodiments, the antisense strand comprises at least about 20 consecutive nucleotides of the nucleotide sequence of any one of SEQ ID NOs: 101-200, 231-260, 288-313, 446-575, 604-637 or 639-644. In some embodiments, the antisense strand comprises at least about 21 consecutive nucleotides of the nucleotide sequence of any one of SEQ ID NOs: 101-200, 231-260, 288-313, 446-575, 604-637 or 639-644. In some embodiments, the antisense strand comprises at least about 22 consecutive nucleotides of the nucleotide sequence of any one of SEQ ID NOs: 101-200, 231-260, 288-313, 446-575, 604-637 or 639-644. In some embodiments, the antisense strand comprises at least about 23 consecutive nucleotides of the nucleotide sequence of any one of SEQ ID NOs: 101-200, 231-260, 288-313, 446-575, 604-637 or 639-644.

In some embodiments, the antisense strand comprises a nucleotide sequence having less than or equal to 5, 4, 3, 2, or 1 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having less than or equal to 5 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having less than or equal to 4 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having less than or equal to 3 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having less than or equal to 2 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having less than or equal to 1 mismatches to the nucleotide sequence of any one of SEQ ID NOs: 1-100, 201-230, 262-287, 314-445, 576-603 or 638 across the entire length of the antisense strand. In some embodiments, the antisense strand comprises a nucleotide sequence having 0 mismatches to the nucleotide sequence of any one of SEQ ID NOs: 1-100, 201-230, 262-287, 314-445, 576-603 or 638 across the entire length of the antisense strand.

In some embodiments, the antisense strand comprises a nucleotide sequence of any of the antisense strands listed in Table 8 or Table 9 or Table 10 or Table 11 or Table 12. In some embodiments, the antisense strand comprises a nucleotide sequence of any of the antisense strands listed in Table 8. In some embodiments, the antisense strand comprises a nucleotide sequence of any of the antisense strands listed in Table 9. In some embodiments, the antisense strand comprises a nucleotide sequence of any of the antisense strands listed in Table 10. In some embodiments, the antisense strand comprises a nucleotide sequence of any of the antisense strands listed in Table 11. In some embodiments, the antisense strand comprises a nucleotide sequence of any of the antisense strands listed in Table 12.

In some embodiments, the antisense strand may comprise an overhang sequence at either the 3′ or 5′ end. In some embodiments, the overhang sequence comprises at least about 1, 2, 3, 4, or 5 or more nucleotides. In some embodiments, the overhang sequence comprises at least about 1 nucleotide. In some embodiments, the overhang sequence comprises at least about 2 nucleotides. In some embodiments, the overhang sequence comprises at least about 3 nucleotides. In some embodiments, the overhang sequence comprises at least about 4 nucleotides. In some embodiments, the overhang sequence comprises at least about 5 nucleotides. In some embodiments, the overhang sequence comprises a UU sequence.

In some embodiments, the antisense strand may comprise at least 1, 2, 3, or 4 phosphorothioate internucleoside linkages. In some embodiments, at least one phosphorothioate internucleoside linkage is between the nucleotides at positions 1 and 2 from the 5′ end of the antisense strand. In some embodiments, at least one phosphorothioate internucleoside linkage is between the nucleotides at positions 2 and 3 from the 5′ end of the antisense strand. In some embodiments, at least one phosphorothioate internucleoside linkage is between the nucleotides at positions 1 and 2 from the 3′ end of the antisense strand. In some embodiments, at least one phosphorothioate internucleoside linkage is between the nucleotides at positions 2 and 3 from the 3′ end of the antisense strand.

In some embodiments, the antisense strand may comprise a nucleotide sequence comprising 2′-fluoro nucleotides at positions 2, 6, 14, and 16 from the 5′ end of the nucleotide sequence. In some embodiments, the antisense strand may comprise a nucleotide sequence comprising 2′-fluoro nucleotides at positions 2 and 14 from the 5′ end of the nucleotide sequence. In some embodiments, the antisense strand may comprise a nucleotide sequence comprising 2′-fluoro nucleotides at positions 2, 5, 8, 14, and 17 from the 5′ end of the nucleotide sequence.

In some embodiments, the antisense strand may comprise a nucleotide sequence consisting of 17 to 23, or 19 to 23, nucleotides, wherein 2′-fluoro nucleotides are at positions 2, 6, 14, and 16 from the 5′ end of the nucleotide sequence. In some embodiments, the antisense strand may comprise a nucleotide sequence consisting of 17 to 23, or 19 to 23, nucleotides, wherein 2′-fluoro nucleotides are at positions 2 and 14 from the 5′ end of the nucleotide sequence. In some embodiments, the antisense strand may comprise a nucleotide sequence consisting of 17 to 23, or 19 to 23, nucleotides, wherein 2′-fluoro nucleotides are at positions 2, 5, 8, 14, and 17 from the 5′ end of the nucleotide sequence. In some embodiments, the antisense strand may comprise a nucleotide sequence consisting of 17 to 23, or 19 to 23, nucleotides, wherein 2′-fluoro nucleotides are at positions 2, 6, 10, 14, and 18.

Modified siRNAs

In some embodiments, the siRNA molecules disclosed herein may be chemically modified. In some embodiments, the siRNA molecules may be modified, for example, to enhance stability and/or bioavailability and/or provide otherwise beneficial characteristics in vitro, in vivo, and/or ex vivo. For example, siRNA molecules may be modified such that the two strands (sense and antisense) maintain the ability to hybridize to each other and/or the siRNA molecules maintain the ability to hybridize to a target sequence. Examples of siRNA modifications include modifications to the ribose sugar, nucleobase, and/or phosphodiester backbone, including but not limited to those described herein. Non-limiting examples of siRNA modifications are described, e.g., in WO 2020/243490; WO 2020/097342; WO 2021/119325; PCT/US2021/019629; PCT/US2021/019628; PCT/US2021/021199; Sig. Transduct. Target Ther. 5 (101), 1-25, 2020; and J. Am. Chem. Soc. 136 (49), 16958-16961, 2014, the contents of each of which are hereby incorporated herein by reference in their entirety.

In some embodiments, the siRNA molecules disclosed herein comprise modified nucleotides having a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′ modifications include, but are not limited to, 2′-OH, 2′-S-alkyl, 2′-N-alkyl, 2′-O-alkyl, 2′-S-alkenyl, 2′-N-alkenyl, 2′-O-alkenyl, 2′-S-alkynyl, 2′-N-alkynyl, 2′-O-alkynyl, 2′-O-allyl, 2′-C-allyl, 2′-fluoro, 2′-O-methyl (OMe or OCH₃), 2′-O-methoxyethyl, 2′-ara-F, 2′-OCF₃, 2′-O(CH₂)₂SCH₃, 2′-O-aminoalkyl, 2′-amino (e.g. NH₂), 2′-O-ethylamine, and 2′-azido, wherein the alkyl, alkenyl and alkynyl can be substituted or unsubstituted. Modifications at the 5′ position of the pentose ring include, but are not limited to, 5′-methyl (R or S), 5′-vinyl, and 5′-methoxy. Sugar modifications may also include, for example, LNA, UNA, GNA, and DNA. In some embodiments, the siRNA molecules of the disclosure comprise one or more 2′-O-methyl nucleotides, 2′-fluoro nucleotides, or combinations thereof.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of any sense or antisense nucleotide sequences described herein are 2′-O-methyl nucleotides. In some embodiments, between about 2 to 20 modified nucleotides of any sense or antisense nucleotide sequences described herein are 2′-O-methyl nucleotides. In some embodiments, between about 5 to 25 modified nucleotides of any sense or antisense nucleotide sequences described herein are 2′-O-methyl nucleotides. In some embodiments, between about 10 to 25 modified nucleotides of any sense or antisense nucleotide sequences described herein are 2′-O-methyl nucleotides. In some embodiments, between about 12 to 25 modified nucleotides of any sense or antisense nucleotide sequences described herein are 2′-O-methyl nucleotides. In some embodiments, at least about 10, 11, 12, 13, 14, 15, 16, 17, 18, 19, 20, 21, or 22 modified nucleotides of any sense or antisense nucleotide sequences described herein are 2′-O-methyl nucleotides. In some embodiments, at least about 12 modified nucleotides of any sense or antisense nucleotide sequences described herein are 2′-O-methyl nucleotides. In some embodiments, at least about 13 modified nucleotides of any sense or antisense nucleotide sequences described herein are 2′-O-methyl nucleotides. In some embodiments, at least about 14 modified nucleotides of any sense or antisense nucleotide sequences described herein are 2′-O-methyl nucleotides. In some embodiments, at least about 15 modified nucleotides of any sense or antisense nucleotide sequences described herein are 2′-O-methyl nucleotides. In some embodiments, at least about 16 modified nucleotides of any sense or antisense nucleotide sequences described herein are 2′-O-methyl nucleotides. In some embodiments, at least about 17 modified nucleotides of any sense or antisense nucleotide sequences described herein are 2′-O-methyl nucleotides. In some embodiments, at least about 18 modified nucleotides of any sense or antisense nucleotide sequences described herein are 2′-O-methyl nucleotides. In some embodiments, at least about 19 modified nucleotides of any sense or antisense nucleotide sequences described herein are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of any sense or antisense nucleotide sequences described herein are 2′-O-methyl nucleotides. In some embodiments, less than or equal to 21 modified nucleotides of any sense or antisense nucleotide sequences described herein are 2′-O-methyl nucleotides. In some embodiments, less than or equal to 20 modified nucleotides of any sense or antisense nucleotide sequences described herein are 2′-O-methyl nucleotides. In some embodiments, less than or equal to 19 modified nucleotides of any sense or antisense nucleotide sequences described herein are 2′-O-methyl nucleotides. In some embodiments, less than or equal to 18 modified nucleotides of any sense or antisense nucleotide sequences described herein are 2′-O-methyl nucleotides. In some embodiments, less than or equal to 17 modified nucleotides of any sense or antisense nucleotide sequences described herein are 2′-O-methyl nucleotides. In some embodiments, less than or equal to 16 modified nucleotides of any sense or antisense nucleotide sequences described herein are 2′-O-methyl nucleotides. In some embodiments, less than or equal to 15 modified nucleotides of any sense or antisense nucleotide sequences described herein are 2′-O-methyl nucleotides. In some embodiments, less than or equal to 14 modified nucleotides of any sense or antisense nucleotide sequences described herein are 2′-O-methyl nucleotides. In some embodiments, less than or equal to 13 modified nucleotides of any sense or antisense nucleotide sequences described herein are 2′-O-methyl nucleotides. In some embodiments, at least one modified nucleotide of any sense or antisense nucleotide sequences described herein is a 2′-O-methyl pyrimidine. In some embodiments, at least 5, 6, 7, 8, 9, or 10 modified nucleotides of any sense or antisense nucleotide sequences described herein are 2′-O-methyl pyrimidines. In some embodiments, at least one modified nucleotide of any sense or antisense nucleotide sequences described herein is a 2′-O-methyl purine. In some embodiments, at least 5, 6, 7, 8, 9, or 10 modified nucleotides of any sense or antisense nucleotide sequences described herein are 2′-O-methyl purines. In some embodiments, the 2′-O-methyl nucleotide is a 2′-O-methyl nucleotide mimic.

In some embodiments, the nucleotide at position 3, 5, 7, 8, 9, 10, 11, 12, 14, 17, and/or 19 from the 5′ end of any sense or antisense nucleotide sequences described herein is a 2′-fluoro nucleotide. In some embodiments, at least two nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least three nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least four nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, at least five nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, the nucleotides at positions 3, 5, 7, 8, 9, 10, 11, 12, 14, 17, and/or 19 from the 5′ end of any sense or antisense nucleotide sequences described herein are 2′-fluoro nucleotides. In some embodiments, the nucleotide at position 3 from the 5′ end of any sense or antisense nucleotide sequences described herein is a 2′-fluoro nucleotide. In some embodiments, the nucleotide at position 7 from the 5′ end of any sense or antisense nucleotide sequences described herein is a 2′-fluoro nucleotide. In some embodiments, the nucleotide at position 8 from the 5′ end of any sense or antisense nucleotide sequences described herein is a 2′-fluoro nucleotide. In some embodiments, the nucleotide at position 9 from the 5′ end of any sense or antisense nucleotide sequences described herein is a 2′-fluoro nucleotide. In some embodiments, the nucleotide at position 12 from the 5′ end of any sense or antisense nucleotide sequences described herein is a 2′-fluoro nucleotide. In some embodiments, the nucleotide at position 17 from the 5′ end of any sense or antisense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 1, 3, 5, 7, 8, 9, 10, 11, 12, 14, 17, and/or 19 from the 5′ end of any sense or antisense nucleotide sequences described herein is a 2′-fluoro nucleotide. In some embodiments, the nucleotide at positions 1, 3, 5, 7, 8, 9, 10, 11, 12, 14, 17, and/or 19 from the 5′ end of any sense or antisense nucleotide sequences described herein is a 2′-fluoro nucleotide. In some embodiments, at least two nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, at least three nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, the nucleotides at positions 1, 3, 5, 7, 8, 9, 10, 11, 12, 14, 17, and/or 19 from the 5′ end of any sense or antisense nucleotide sequences described herein are 2′-fluoro nucleotides. In some embodiments, the 2′-fluoro nucleotide is a 2′-fluoro nucleotide mimic.

In some embodiments, at least 1, 2, 3, 4, 5, 6, or 7 nucleotides at position 2, 4, 6, 8, 10, 12, 14, 16, and/or 18 from the 5′ end of any sense or antisense nucleotide sequences described herein is a 2′-fluoro nucleotide. In some embodiments, the nucleotide at positions, 4, 6, 8, 10, 12, 14, 16, and/or 18 from the 5′ end of any sense or antisense nucleotide sequences described herein is a 2′-fluoro nucleotide. In some embodiments, at least two nucleotides at positions 2, 4, 6, 8, 10, 12, 14, 16, and/or 18 from the 5′ end of any sense or antisense nucleotide sequences described herein are 2′-fluoro nucleotides. In some embodiments, at least three nucleotides at positions 2, 4, 6, 8, 10, 12, 14, 16, and/or 18 from the 5′ end of any sense or antisense nucleotide sequences described herein are 2′-fluoro nucleotides. In some embodiments, the nucleotides at positions 2, 4, 6, 8, 10, 12, 14, 16, and/or 18 from the 5′ end of any sense or antisense nucleotide sequences described herein are 2′-fluoro nucleotides. In some embodiments, the 2′-fluoro nucleotide is a 2′-fluoro nucleotide mimic.

In some embodiments, the nucleotide at position 1 from the 5′ end of any sense nucleotide sequences described herein is a 2′-fluoro nucleotide. In some embodiments, the nucleotide at position 3 from the 5′ end of any sense nucleotide sequences described herein is a 2′-fluoro nucleotide. In some embodiments, the nucleotide at position 5 from the 5′ end of any sense nucleotide sequences described herein is a 2′-fluoro nucleotide. In some embodiments, the nucleotide at position 7 from the 5′ end of any sense nucleotide sequences described herein is a 2′-fluoro nucleotide. In some embodiments, the nucleotide at position 8 from the 5′ end of any sense nucleotide sequences described herein is a 2′-fluoro nucleotide. In some embodiments, the nucleotide at position 9 from the 5′ end of any sense nucleotide sequences described herein is a 2′-fluoro nucleotide. In some embodiments, the nucleotide at position 10 from the 5′ end of any sense nucleotide sequences described herein is a 2′-fluoro nucleotide. In some embodiments, the nucleotide at position 11 from the 5′ end of any sense nucleotide sequences described herein is a 2′-fluoro nucleotide. In some embodiments, the nucleotide at position 12 from the 5′ end of any sense nucleotide sequences described herein is a 2′-fluoro nucleotide. In some embodiments, the nucleotide at position 14 from the 5′ end of any sense nucleotide sequences described herein is a 2′-fluoro nucleotide. In some embodiments, the nucleotide at position 17 from the 5′ end of any sense nucleotide sequences described herein is a 2′-fluoro nucleotide. In some embodiments, the nucleotide at position 19 from the 5′ end of any sense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 1, 3, 5, 7, 8, 9, 10, 11, 12, 14, 17, and/or 19 from the 5′ end of any sense nucleotide sequences described herein is a 2′-fluoro nucleotide. In some embodiments, the nucleotide at position 5, 7, 8, 9, 10, 11, 14, and/or 19 from the 5′ end of any sense nucleotide sequences described herein is a 2′-fluoro nucleotide. In some embodiments, the nucleotide at position 5, 7, 8, and/or 9 from the 5′ end of any sense nucleotide sequences described herein is a 2′-fluoro nucleotide. In some embodiments, the nucleotide at position 7, 9, 10, and/or 11 from the 5′ end of any sense or antisense nucleotide sequences described herein is a 2′-fluoro nucleotide. In some embodiments, the nucleotide at position 5, 9, 10, 11, 14, and/or 19 from the 5′ end of any sense nucleotide sequences described herein is a 2′-fluoro nucleotide. In some embodiments, the 2′-fluoro nucleotide is a 2′-fluoro nucleotide mimic.

In some embodiments, the nucleotide at position 2 from the 5′ end of any antisense nucleotide sequences described herein is a 2′-fluoro nucleotide. In some embodiments, the nucleotide at position 4 from the 5′ end of any antisense nucleotide sequences described herein is a 2′-fluoro nucleotide. In some embodiments, the nucleotide at position 6 from the 5′ end of any antisense nucleotide sequences described herein is a 2′-fluoro nucleotide. In some embodiments, the nucleotide at position 8 from the 5′ end of any antisense nucleotide sequences described herein is a 2′-fluoro nucleotide. In some embodiments, the nucleotide at position 10 from the 5′ end of any antisense nucleotide sequences described herein is a 2′-fluoro nucleotide. In some embodiments, the nucleotide at position 12 from the 5′ end of any antisense nucleotide sequences described herein is a 2′-fluoro nucleotide. In some embodiments, the nucleotide at position 14 from the 5′ end of any antisense nucleotide sequences described herein is a 2′-fluoro nucleotide. In some embodiments, the nucleotide at position 16 from the 5′ end of any antisense nucleotide sequences described herein is a 2′-fluoro nucleotide. In some embodiments, the nucleotide at position 18 from the 5′ end of any antisense nucleotide sequences described herein is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 2, 4, 5, 6, 8, 10, 12, 14, 16, 17 and/or 18 from the 5′ end of any antisense nucleotide sequences described herein is a 2′-fluoro nucleotide. In some embodiments, the nucleotide at position 2, 5, 6, 8, 14, 16, and/or 17 from the 5′ end of any antisense nucleotide sequences described herein is a 2′-fluoro nucleotide. In some embodiments, the nucleotide at position 2, 6, 14, and/or 16 from the 5′ end of any antisense nucleotide sequences described herein is a 2′-fluoro nucleotide. In some embodiments, the nucleotide at position 2, and/or 14 from the 5′ end of any antisense nucleotide sequences described herein is a 2′-fluoro nucleotide. In some embodiments, the nucleotide at position 2, 5, 8, 14, and/or 17 from the 5′ end of any antisense nucleotide sequences described herein is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

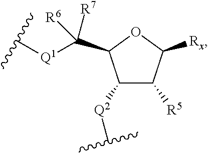

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

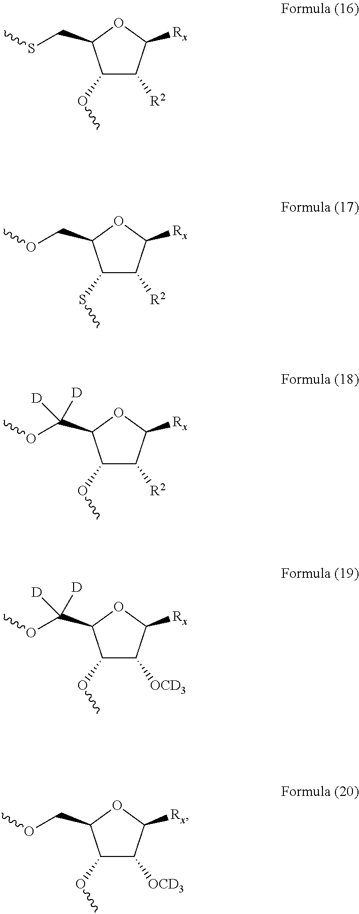

wherein Rₓ is independently a nucleobase and R² is F or —OCH₃. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure

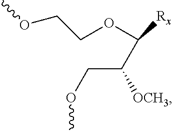

wherein Rₓ is a nucleobase, aryl, heteroaryl, or H. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure:

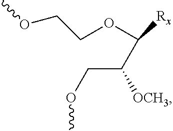

wherein Rₓ is a nucleobase. In some embodiments, the nucleobase is selected from thymine, cytosine, guanine, adenine, uracil, and an analogue or derivative thereof.

In some embodiments, the sense strand or the antisense strand may comprise at least 1, at least 2, at least 3, at least 4, or at least 5 or more modified nucleotide(s) having the following chemical structure:

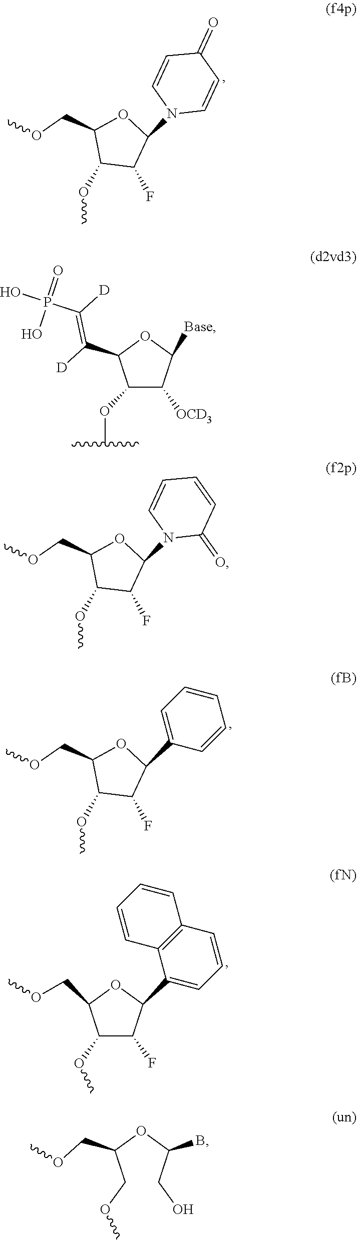

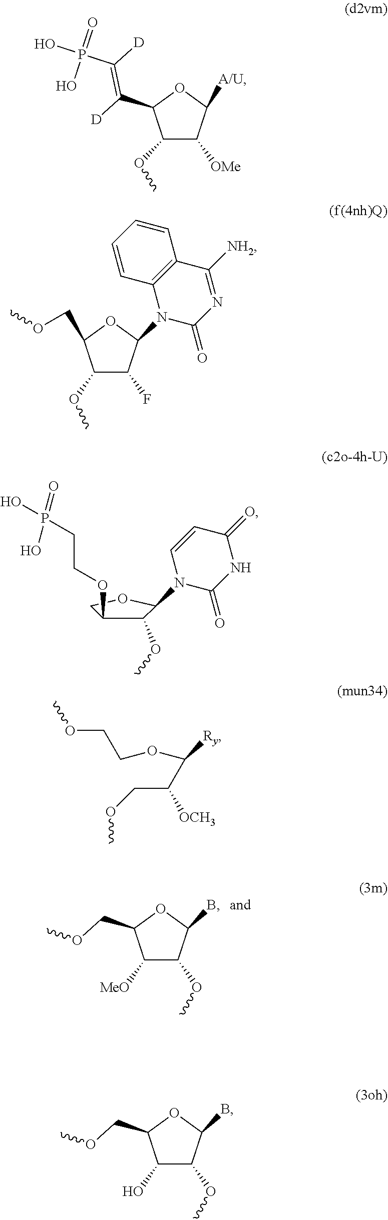

wherein B and R_{y} is a nucleobase. In some embodiments, the nucleobase is selected from thymine, cytosine, guanine, adenine, uracil, and an analogue or derivative thereof.

In some embodiments, any sense or antisense nucleotide sequence described herein comprises, consists of, or consists essentially of ribonucleic acids (RNAs). In some embodiments, any sense or antisense nucleotide sequence described herein comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of any sense or antisense nucleotide sequence described herein are independently selected from 2′-O-methyl RNA and 2′-fluoro RNA.

In some embodiments, the siRNA molecules disclosed herein include end modifications at the 5′ end and/or the 3′ end of the sense strand and/or the antisense strand. In some embodiments, the siRNA molecules disclosed herein comprise a phosphate moiety at the 5′ end of the sense strand and/or antisense strand. In some embodiments, the 5′ end of the sense strand and/or antisense strand comprises a phosphate mimic or analogue (e.g., “5′ terminal phosphate mimic”). In some embodiments, the 5′ end of the sense strand and/or antisense strand comprises a vinyl phosphonate or a variation thereof (e.g., “5′ terminal vinyl phosphonate”).

In some embodiments, the siRNA molecules comprise at least one backbone modification, such as a modified internucleoside linkage. In some embodiments, the siRNA molecules described herein comprise at least one phosphorothioate internucleoside linkage. In particular embodiments, the phosphorothioate internucleoside linkages may be positioned at the 3′ or 5′ ends of the sense and/or antisense strands.

In some embodiments, siRNA molecules include an overhang of at least one unpaired nucleotide. In some embodiments in which the siRNA molecule comprises a nucleotide overhang, two or more of the unpaired nucleotides in the overhang can be connected by a phosphorothioate internucleoside linkage. In certain embodiments, all the unpaired nucleotides in a nucleotide overhang at the 3′ end of the antisense strand and/or the sense strand are connected by phosphorothioate internucleoside linkages. In some embodiments, all the unpaired nucleotides in a nucleotide overhang at the 5′ end of the antisense strand and/or the sense strand are connected by phosphorothioate internucleoside linkages. In some embodiments, all of the unpaired nucleotides in any nucleotide overhang are connected by phosphorothioate internucleoside linkages.

In some embodiments, the sense or the antisense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of any sense or antisense nucleotide sequences described herein. In some embodiments, at least one phosphorothioate internucleoside linkage is between the nucleotides at positions 2 and 3 from the 5′ end of any sense or antisense nucleotide sequences described herein. In some embodiments, the sense strand comprises two phosphorothioate internucleoside linkages between the nucleotides at positions 1 to 3 from the 5′ end of any sense or antisense nucleotide sequences described herein.

In some embodiments, the modified nucleotides that can be incorporated into the siRNA molecules of the disclosure may have more than one chemical modification described herein. For instance, in some embodiments, the modified nucleotide may have a modification to the ribose sugar as well as a modification to the phosphodiester backbone. By way of example, a modified nucleotide may comprise a 2′ sugar modification (e.g., 2′-fluoro or 2′-O-methyl) and a modification to the 5′ phosphate that would create a modified internucleoside linkage when the modified nucleotide was incorporated into a polynucleotide. For instance, in some embodiments, the modified nucleotide may comprise a sugar modification, such as a 2′-fluoro modification or a 2′-O-methyl modification, for example, as well as a 5′ phosphorothioate group. In some embodiments, the sense and/or antisense strand of the siRNA molecules of the disclosure comprises a combination of 2′ modified nucleotides and phosphorothioate internucleoside linkages. In some embodiments, the sense and/or antisense strand of the siRNA molecules of the disclosure comprises a combination of 2′ sugar modifications, phosphorothioate internucleoside linkages, and 5′ terminal vinyl phosphonate.

In some embodiments, any of the siRNAs disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the siRNAs disclosed herein comprise 1 or more modified nucleotides. In some embodiments, any of the siRNAs disclosed herein comprise 2 or more modified nucleotides. In some embodiments, any of the siRNAs disclosed herein comprise 5 or more modified nucleotides. In some embodiments, any of the siRNAs disclosed herein comprise 8 or more modified nucleotides. In some embodiments, any of the siRNAs disclosed herein comprise 10 or more modified nucleotides. In some embodiments, any of the siRNAs disclosed herein comprise 15 or more modified nucleotides. In some embodiments, any of the siRNAs disclosed herein comprise 20 or more modified nucleotides. In some embodiments, any of the siRNAs disclosed herein comprise 30 or more modified nucleotides. In some embodiments, any of the siRNAs disclosed herein comprise 35 or more modified nucleotides. In some embodiments, any of the siRNAs disclosed herein comprise 40 or more modified nucleotides. In some embodiments, any of the siRNAs disclosed herein comprise 45 or more modified nucleotides. In some embodiments, all of the nucleotides in the siRNA molecule are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, any of the sense strands disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the sense strands disclosed herein comprise 1 or more modified nucleotides. In some embodiments, any of the sense strands disclosed herein comprise 2 or more modified nucleotides. In some embodiments, any of the sense strands disclosed herein comprise 5 or more modified nucleotides. In some embodiments, any of the sense strands disclosed herein comprise 8 or more modified nucleotides. In some embodiments, any of the sense strands disclosed herein comprise 10 or more modified nucleotides. In some embodiments, any of the sense strands disclosed herein comprise 15 or more modified nucleotides. In some embodiments, any of the sense strands disclosed herein comprise 17 or more modified nucleotides. In some embodiments, any of the sense strands disclosed herein comprise 18 or more modified nucleotides. In some embodiments, any of the sense strands disclosed herein comprise 19 or more modified nucleotides. In some embodiments, any of the sense strands disclosed herein comprise 20 or more modified nucleotides. In some embodiments, any of the sense strands disclosed herein comprise 21 or more modified nucleotides. In some embodiments, all of the nucleotides in the sense strand are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, any of the antisense strands disclosed herein comprise 1, 2, 3, 4, 5, 6, 7, 8, 9, 10, 11, 12, 13, 14, 15, 16, 17, 18, 19, 20, 21, 22, 23, 24, 25, or more modified nucleotides. In some embodiments, any of the antisense strands disclosed herein comprise 1 or more modified nucleotides. In some embodiments, any of the antisense strands disclosed herein comprise 2 or more modified nucleotides. In some embodiments, any of the antisense strands disclosed herein comprise 5 or more modified nucleotides. In some embodiments, any of the antisense strands disclosed herein comprise 8 or more modified nucleotides. In some embodiments, any of the antisense strands disclosed herein comprise 10 or more modified nucleotides. In some embodiments, any of the antisense strands disclosed herein comprise 15 or more modified nucleotides. In some embodiments, any of the antisense strands disclosed herein comprise 17 or more modified nucleotides. In some embodiments, any of the antisense strands disclosed herein comprise 18 or more modified nucleotides. In some embodiments, any of the antisense strands disclosed herein comprise 19 or more modified nucleotides. In some embodiments, any of the antisense strands disclosed herein comprise 20 or more modified nucleotides. In some embodiments, any of the antisense strands disclosed herein comprise 21 or more modified nucleotides. In some embodiments, any of the antisense strands disclosed herein comprise 22 or more modified nucleotides. In some embodiments, any of the antisense strands disclosed herein comprise 23 or more modified nucleotides. In some embodiments, all of the nucleotides in the antisense strand are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, at least about 10%, 20%, 30%, 40%, 45%, 50%, 55%, 60%, 65%, 70%, 75%, 80%, 90%, 95%, or 100% of the nucleotides in any of the sense strands disclosed herein are modified nucleotides. In some embodiments, at least about 10% of the nucleotides in any of the sense strands disclosed herein are modified nucleotides. In some embodiments, at least about 30% of the nucleotides in any of the sense strands disclosed herein are modified nucleotides. In some embodiments, at least about 50% of the nucleotides in any of the sense strands disclosed herein are modified nucleotides. In some embodiments, at least about 60% of the nucleotides in any of the sense strands disclosed herein are modified nucleotides. In some embodiments, at least about 70% of the nucleotides in any of the sense strands disclosed herein are modified nucleotides. In some embodiments, at least about 80% of the nucleotides in any of the sense strands disclosed herein are modified nucleotides. In some embodiments, at least about 90% of the nucleotides in any of the sense strands disclosed herein are modified nucleotides. In some embodiments, at least about 100% of the nucleotides in any of the sense strands disclosed herein are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

In some embodiments, at least about 10%, 20%, 30%, 40%, 45%, 50%, 55%, 60%, 65%, 70%, 75%, 80%, 90%, 95%, or 100% of the nucleotides in any of the antisense strands disclosed herein are modified nucleotides. In some embodiments, at least about 10% of the nucleotides in any of the antisense strands disclosed herein are modified nucleotides. In some embodiments, at least about 30% of the nucleotides in any of the antisense strands disclosed herein are modified nucleotides. In some embodiments, at least about 50% of the nucleotides in any of the antisense strands disclosed herein are modified nucleotides. In some embodiments, at least about 60% of the nucleotides in any of the antisense strands disclosed herein are modified nucleotides. In some embodiments, at least about 70% of the nucleotides in any of the antisense strands disclosed herein are modified nucleotides. In some embodiments, at least about 80% of the nucleotides in any of the antisense strands disclosed herein are modified nucleotides. In some embodiments, at least about 90% of the nucleotides in any of the antisense strands disclosed herein are modified nucleotides. In some embodiments, at least about 100% of the nucleotides in any of the antisense strands disclosed herein are modified nucleotides. In some embodiments, the one or more modified nucleotides is independently selected from a 2′-O-methyl nucleotide, a 2′-fluoro nucleotide, a locked nucleic acid, a nucleoside analog, a 5′ terminal vinyl phosphonate, and a 5′ phosphorothioate.

siRNA Conjugates

In some embodiments, the siRNA molecules disclosed herein may comprise one or more conjugates or ligands. As used herein, a “conjugate” or “ligand” refers to any compound or molecule that is capable of interacting with another compound or molecule, directly or indirectly. In some embodiments, the ligand may modify one or more properties of the siRNA molecule to which it is attached, such as the pharmacodynamic, pharmacokinetic, binding, absorption, cellular distribution, cellular uptake, charge and/or clearance properties of the siRNA molecule. Non-limiting examples of such conjugates are described, e.g., in WO 2020/243490; WO 2020/097342; WO 2021/119325; PCT/US2021/019629; PCT/US2021/019628; PCT/US2021/021199; Sig. Transduct. Target Ther. 5 (101), 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, each of which is incorporated by reference herein.

In some embodiments, the ligand may be attached to the 5′ end and/or the 3′ end of the sense and/or antisense strand of the siRNA via covalent attachment such as to a nucleotide. In some embodiments, the ligand is covalently attached via a linker to the sense or antisense strand of the siRNA molecule. The ligand can be attached to nucleobases, sugar moieties, or internucleoside linkages of polynucleotides (e.g., sense strand or antisense strand) of the siRNA molecules of the disclosure.

In some embodiments, the type of conjugate or ligand used and the extent of conjugation of siRNA molecules of the disclosure can be evaluated, for example, for improved pharmacokinetic profiles, bioavailability, and/or stability of siRNA molecules while at the same time maintaining the ability of the siRNA to mediate RNAi activity. In some embodiments, a conjugate or ligand alters the distribution, targeting or lifetime of a siRNA molecule into which it is incorporated. In some embodiments, a conjugate or ligand provides an enhanced affinity for a selected target, e.g., molecule, cell or cell type, compartment (e.g., a cellular or organ compartment), tissue, organ or region of the body, as, e.g., compared to a molecule absent such a ligand.

In some embodiments, a conjugate or ligand can include a naturally occurring substance or a recombinant or synthetic molecule. Non-limiting examples of conjugates and ligands include serum proteins (e.g., human serum albumin, low-density lipoprotein, globulin), cholesterol moieties, vitamins (e.g., biotin, vitamin E, vitamin B12), folate moieties, steroids, bile acids (e.g., cholic acid), fatty acids (e.g., palmitic acid, myristic acid), carbohydrates (e.g., a dextran, pullulan, chitin, chitosan, inulin, cyclodextrin, hyaluronic acid, or N-acetyl-galactosamine (GalNAc)), glycosides, phospholipids, antibodies or binding fragment thereof (e.g., antibody or binding fragment that targets the siRNA to a specific cell type, such as liver), a dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules (e.g., cholesterol, tocopherol, long fatty acids (e.g., docosanoic, palmitoyl, docosahexaenoic), cholic acid, adamantane acetic acid, 1-pyrene butyric acid, dihydrotestosterone, 1,3-BisO (hexadecyl)glycerol, geranyloxyhexyl group, hexadecylglycerol, borneol, menthol, 1,3-propanediol, heptadecyl group, 03-(oleoyl) lithocholic acid, 03-(oleoyl) cholenic acid, dimethoxytrityl, or phenoxazine), peptides (e.g., antennapedia peptide, Tat peptide, RGD peptides), alkylating agents, polymers, such as polyethylene glycol (PEG) (e.g., PEG-40K), poly amino acids, polyamines (e.g., spermine, spermidine), alkyls, substituted alkyls, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some embodiments, the conjugate or ligand comprises a carbohydrate. Carbohydrates include, but are not limited to, sugars (e.g., monosaccharides, disaccharides, trisaccharides, tetrasaccharides, and oligosaccharides containing from about 4, 5, 6, 7, 8, or 9 monosaccharide units) and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units.

In some embodiments, the carbohydrate incorporated into the conjugate or ligand is an amino sugar, such as galactosamine, glucosamine, N-acetyl-galactosamine (GalNAc), and N-acetyl-glucosamine. In some embodiments, the conjugate or ligand comprises N-acetyl-galactosamine and derivatives thereof. Non-limiting examples of GalNAc- or galactose-containing ligands that can be incorporated into the siRNAs of the disclosure are described in WO 2020/243490; WO 2020/097342; WO 2021/119325; PCT/US2021/019629; PCT/US2021/019628; PCT/US2021/021199; Sig. Transduct. Target Ther. 5 (101), 1-25, 2020; ACS Chem. Biol. 10 (5), 1181-1187, 2015; J. Am. Chem. Soc. 136 (49), 16958-16961, 2014; Nucleic Acids Res. 42 (13), 8796-8807, 2014; Molec. Ther. 28 (8), 1759-1771, 2020; and Nucleic Acid Ther. 28 (3), 109-118, 2018, all of which are hereby incorporated herein by reference in their entireties.

The conjugate or ligand can be attached or conjugated to the siRNA molecule directly or indirectly. For instance, in some embodiments, the ligand is covalently attached directly to the sense or antisense strand of the siRNA molecule. In other embodiments, the ligand is covalently attached via a linker to the sense or antisense strand of the siRNA molecule. The ligand can be attached to nucleobases, sugar moieties, or internucleoside linkages of polynucleotides (e.g. sense strand or antisense strand) of the siRNA molecules of the disclosure. In some embodiments, the conjugate or ligand may be attached to the 5′ end and/or to the 3′ end of the sense and/or antisense strand of the siRNA molecule. In certain embodiments, the ligand is covalently attached to the 5′ end of the sense strand. In some embodiments, the ligand is covalently attached to the 3′ end of the sense strand. In some embodiments, the ligand is attached to the 5′ terminal nucleotide of the sense strand or the 3′ terminal nucleotide of the sense strand.

In some embodiments, the conjugate or ligand covalently attached to the sense and/or antisense strand of the siRNA molecule comprises a GalNAc derivative. In some embodiments, the GalNAc derivative is attached to the 5′ end and/or to the 3′ end of the sense and/or antisense strand of the siRNA molecule. In some embodiments, the GalNAc derivative is attached to the 3′ end of the sense strand. In some embodiments, the GalNAc derivative is attached to the 5′ end of the sense strand. In some embodiments, the GalNAc derivative is attached to the 3′ end of the antisense strand. In some embodiments, the GalNAc derivative is attached to the 5′ end of the antisense strand. In some embodiments, the GalNAc derivative is attached to the 5′ end of the sense strand and to the 3′ end of the sense strand.

In some embodiments, the conjugate or ligand is a GalNAc derivative comprising 1, 2, 3, 4, 5, or 6 monomeric GalNAc units. In some embodiments, the conjugate or ligand is a GalNAc derivative comprising 1 monomeric GalNAc units. In some embodiments, the conjugate or ligand is a GalNAc derivative comprising 2 monomeric GalNAc units. In some embodiments, the conjugate or ligand is a GalNAc derivative comprising 3 monomeric GalNAc units. In some embodiments, the conjugate or ligand is a GalNAc derivative comprising 4 monomeric GalNAc units. In some embodiments, the conjugate or ligand is a GalNAc derivative comprising 5 monomeric GalNAc units. In some embodiments, the conjugate or ligand is a GalNAc derivative comprising 6 monomeric GalNAc units. In some embodiments, a various amounts of monomeric GalNAc units are attached at the 5′ end and the 3′ end of the sense strand. In some embodiments, a various amounts of monomeric GalNAc units are attached at the 5′ end and the 3′ end of the antisense strand. In some embodiments, 1, 2, 3, 4, 5, or 6 monomeric GalNAc units are attached at the 5′ end of the sense strand. In some embodiments, 1, 2, 3, 4, 5, or 6 monomeric GalNAc units are attached at the 3′ end of the sense strand. In some embodiments, 1, 2, 3, 4, 5, or 6 monomeric GalNAc units are attached at the 5′ end of the antisense strand. In some embodiments, 1, 2, 3, 4, 5, or 6 monomeric GalNAc units are attached at the 3′ end of the antisense strand. In some embodiments, the same number of monomeric GalNAc units are attached at both the 5′ end and the 3′ end of the sense strand. In some embodiments, the same number of monomeric GalNAc units are attached at both the 5′ end and the 3′ end of the antisense strand. In some embodiments, different number of monomeric GalNAc units are attached at the 5′ end and the 3′ end of the sense strand. In some embodiments, different number of monomeric GalNAc units are attached at the 5′ end and the 3′ end of the antisense strand.

In some embodiments, the double stranded siRNA molecule of any one of siRNA Duplex ID Nos. ds-siNA D1-D178 or mds-siNA MD1-MD178, further comprises a GalNAc derivative attached to the 5′ end and/or to the 3′ end of the sense and/or antisense strand of the siRNA molecule. In some embodiments, the double stranded siRNA molecule selected from any one of the siRNA Duplexes of Table 8 or Table 9 or Table 10 or Table 11 or Table 12 further comprises a GalNAc derivative attached to the 5′ end and/or to the 3′ end of the sense and/or antisense strand of the siRNA molecule.

### HSD17B13

In some embodiments, any of the siRNAs disclosed herein specifically downregulate expression of HSD17B13 gene or a variant thereof. In some embodiments, any of the siRNAs disclosed herein specifically downregulate expression of HSD17B13 gene or a variant thereof in a cell by at least about 10%, 15%, 20%, 25%, 30%, 35%, 40%, 45%, 50%, 55%, 60%, 65%, 70%, 75%, 80%, 85%, 90%, 95%, or 100%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 30%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 50%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 60%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 70%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 75%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 80%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 85%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 90%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 95%, wherein the percent of downregulation of expression is compared to a cell not contacted with the siRNA. In some embodiments, any of the siRNAs disclosed herein specifically downregulate expression of HSD17B13 gene or a variant thereof in a cell by at least about 100%, wherein the percent of downregulation of expression is compared to a cell not contacted with the siRNA.

The expression of HSD17B13 gene is measured by any method known in the art. Exemplary methods for measuring expression of HSD17B13 gene include, but are not limited to, quantitative PCR, RT-PCR, RT-qPCR, western blot, Southern blot, northern blot, FISH, DNA microarray, tiling array, and RNA-Seq. The expression of the HSD17B13 gene may be assessed, for example, based on the level, or the change in the level, of any variable associated with HSD17B13 gene expression, e.g., HSD17B13 mRNA level, HSD17B13 protein level, and/or the number or extent of amyloid deposits. This level may be assessed, for example, in an individual cell or in a group of cells, including, for example, a sample derived from a subject. In some embodiments, downregulation or inhibition may be assessed by a decrease in an absolute or relative level of one or more variables that are associated with HSD17B13 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive or attenuated agent control).

In some embodiments, the HSD17B13 gene comprises a nucleotide sequence that is at least about 80%, 85%, 90%, 91%, 92%, 93%, 94%, 95%, 96%, 97%, 98%, 99%, or 100% identical to the nucleotide sequence of SEQ ID NO: 1 across the full-length of SEQ ID NO: 261 (GenBank Accession No. NM_178135.5 (nucleotides 42 to 944)).

In some embodiments, the HSD17B13 gene comprises a nucleotide sequence having less than or equal to 1, 2, 3, 4, 5, 6, 7, 8, 9, 10, 11, 12, 13, 14, 15, 16, 17, 18, 19, or 20 nucleotide mismatches to the nucleotide sequence of SEQ ID NO: 261 across the full-length of SEQ ID NO: 261.

In some embodiments, the fragment of the HSD17B13 gene is about 10 to about 50, or about 15 to about 50, or about 15 to about 45 nucleotides, or about 15 to about 40, or about 15 to about 35, or about 15 to about 30, or about 15 to about 25, or about 17 to about 23 nucleotides, or about 17 to about 22, or about 17 to about 21, or about 18 to about 23, or about 18 to about 22, or about 18 to about 21, or about 19 to about 23, or about 19 to about 22, or about 19 to about 21 nucleotides in length.

Administration of siRNA

Administration of any of the siRNAs disclosed herein may be conducted by methods known in the art, including as described below. The siRNAs of the present disclosure may be given systemically or locally, for example, orally, nasally, parenterally, topically, intracisternally, intravaginally, or rectally, and are given in forms suitable for each administration route.

The delivery of a siRNA molecule of the disclosure to a cell, e.g., a cell within a subject, such as a human subject (e.g., a subject in need thereof, including a subject having a disease, disorder or condition associated with HSD17B13 gene expression) can be achieved in a number of different ways. For example, in some embodiments, delivery may be performed by contacting a cell with a siRNA of the disclosure either in vitro, in vivo, or ex vivo. In some embodiments, in vivo delivery may be performed, for example, by administering a pharmaceutical composition comprising a siRNA molecule to a subject. In some embodiments, in vivo delivery may be performed by administering one or more vectors that encode and direct the expression of the siRNA.

In general, any method of delivering a nucleic acid molecule (in vitro, in vivo, or ex vivo) can be adapted for use with a siRNA molecule of the disclosure. For in vivo delivery, factors to consider in order to deliver a siRNA molecule include, for example, biological stability of the delivered molecule, prevention of non-specific effects, and accumulation of the delivered molecule in the target tissue and non-target tissue.

In some embodiments, the non-specific effects of a siRNA can be minimized by local administration, for example, by direct injection or implantation into a tissue or topically administering the preparation. Local administration to a treatment site can, for example, maximize the local concentration of the agent, limit the exposure of the agent to systemic tissues that can otherwise be harmed by the agent or that can degrade the agent, and permit a lower total dose of the siRNA molecule to be administered.

In some embodiments, the siRNAs or pharmaceutical compositions comprising the siRNAs of the disclosure can be locally administered to relevant tissues ex vivo, or in vivo through, for example, injection, infusion pump or stent, with or without their incorporation in biopolymers.

For administering a siRNA for the treatment of a disease, the siRNA can be modified or alternatively delivered using a drug delivery system; both methods can act, for example, to prevent the rapid degradation of the dsRNA by endo- and exo-nucleases in vivo. Modification of the siRNA or the pharmaceutical carrier can also permit targeting of the siRNA composition to the target tissue and avoid undesirable off-target effects. For example, siRNA molecules can be modified by conjugation to lipophilic groups such as cholesterol as described above to, e.g., enhance cellular uptake and prevent degradation.

In some embodiments, the siRNA can be delivered using drug delivery systems such as a nanoparticle, a dendrimer, a polymer, liposomes, or a cationic delivery system. Positively charged cationic delivery systems can facilitate binding of a siRNA molecule (negatively charged) and also enhance interactions at the negatively charged cell membrane to permit efficient uptake of a siRNA by the cell. In some embodiments, cationic lipids, dendrimers, or polymers can either be bound to a siRNA, or induced to form a vesicle or micelle that encases a siRNA. The formation of vesicles or micelles may further prevent degradation of the siRNA when administered systemically, for example.

Some non-limiting examples of drug delivery systems useful for systemic delivery of siRNAs include DOTAP, cardiolipin, polyethyleneimine, Arg-Gly-Asp (RGD) peptides, and polyamidoamines. In some embodiments, a siRNA forms a complex with cyclodextrin for systemic administration.

### Pharmaceutical Compositions

The siRNA molecules of the disclosure can be administered to animals, including to mammals, and in particular to humans, as pharmaceuticals by themselves, in mixtures with one another, and/or in the form of pharmaceutical compositions.

The present disclosure includes pharmaceutical compositions and formulations which include the siRNA molecules of the disclosure. In some embodiments, a siRNA molecule of the disclosure may be administered in a pharmaceutical composition. In some embodiments, the pharmaceutical compositions of the disclosure comprise one or more siRNA molecules of the disclosure and a pharmaceutically acceptable carrier. When reference is made in the present disclosure to a siRNA molecule, it is to be understood that reference is also made to a pharmaceutical composition containing the siRNA molecule, if appropriate.

In some embodiments, the pharmaceutical composition comprises at least 1, 2, 3, 4, 5, 6, 7, 8, 9, 10, 11, 12, 13, 14, 15, 16, 17, 18, 19, or 20 or more of any of the siRNA molecules disclosed herein.

In some embodiments, any of the pharmaceutical compositions disclosed herein comprise one or more excipients, carriers, wetting agents, diluents, emulsifiers, lubricants, coloring agents, release agents, coating agents, sweetening, flavoring and perfuming agents, preservatives and antioxidants.

In some embodiments, a siRNA molecule of the disclosure may be administered in “naked” form, where the modified or unmodified siRNA molecule is directly suspended in aqueous or suitable buffer solvent, as a “free siRNA.” The free siRNA may be in a suitable buffer solution, which may comprise, for example, acetate, citrate, prolamine, carbonate, or phosphate, or any combination thereof. In one embodiment, the buffer solution is phosphate buffered saline (PBS). The pH and osmolality of the buffer solution containing the siRNA can be adjusted such that it is suitable for administering to a subject.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain embodiments, a pharmaceutical composition of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siRNA molecule) of the present disclosure. In certain embodiments, an aforementioned composition renders orally bioavailable a siRNA molecule of the present disclosure.

Methods of preparing these formulations or pharmaceutical compositions include, for example, the step of bringing into association a siRNA molecule of the present disclosure with the carrier and, optionally, one or more accessory ingredients. In general, the formulations are prepared by uniformly and intimately bringing into association a siRNA molecule of the present disclosure with liquid carriers, or finely divided solid carriers, or both, and then, if necessary, shaping the product.

Administration of the pharmaceutical compositions of the present disclosure may be via any common route, and they are given in forms suitable for each administration route. Such routes include, but are not limited to, parenteral (e.g., subcutaneous, intramuscular, intraperitoneal or intravenous), oral, nasal, airway (e.g., aerosol), buccal, intradermal, transdermal, sublingual, rectal, and vaginal. In some embodiments, administration is by direct injection into liver tissue or delivery through the hepatic portal vein. In some embodiments, the pharmaceutical composition is administered orally. In some embodiments, the pharmaceutical composition is administered parenterally. In some embodiments, the compositions are administered by subcutaneous or intravenous infusion or injection. In some embodiments, the pharmaceutical composition is administered subcutaneously.

Pharmaceutical compositions of the disclosure suitable for oral administration may be, for example, in the form of capsules (e.g., hard or soft capsules), cachets, pills, tablets, lozenges (using a flavored basis, usually, e.g., sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a siRNA molecule of the present disclosure as an active ingredient. A siRNA molecule of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as, for example, sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made, for example, by compression or molding, optionally with one or more accessory ingredients. Compressed tablets may be prepared, for example, using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made, for example, by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the siRNA molecules of the disclosure include, for example, pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as, for example, wetting agents, emulsifying and suspending agents, sweetening, flavoring, coloring, perfuming and preservative agents.

Suspensions, in addition to the siRNA molecules, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more siRNA molecules of the disclosure with one or more suitable nonirritating excipients or carriers comprising, for example, cocoa butter, polyethylene glycol, a suppository wax or a salicylate, and which, for example, is solid at room temperature, but liquid at body temperature and, therefore, will melt in the rectum or vaginal cavity and release the siRNA molecule.

Formulations of the present disclosure which are suitable for vaginal administration also include, for example, pessaries, tampons, creams, gels, pastes, foams or spray formulations containing such carriers as are known in the art to be appropriate.

Dosage forms for the topical or transdermal administration of a siRNA molecule of this disclosure include, for example, powders, sprays, ointments, pastes, creams, lotions, gels, solutions, patches and inhalants. The siRNA molecule may be mixed under sterile conditions with a pharmaceutically acceptable carrier, and with any preservatives, buffers, or propellants which may be required.

The ointments, pastes, creams and gels may contain, in addition to an active siRNA molecule of this disclosure, excipients, such as, for example, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a siRNA molecule of this disclosure, excipients such as, for example, lactose, talc, silicic acid, aluminum hydroxide, calcium silicates and polyamide powder, or mixtures of these substances. Sprays can additionally contain customary propellants, such as, for example, chlorofluorohydrocarbons and volatile unsubstituted hydrocarbons, such as butane and propane.

Transdermal patches have the added advantage of providing controlled delivery of a siRNA molecule) of the present disclosure to the body. Such dosage forms can be made by dissolving or dispersing the siRNA molecule in the proper medium. Absorption enhancers can also be used to increase the flux of the siRNA molecule across the skin. The rate of such flux can be controlled, for example, by either providing a rate controlling membrane or dispersing the siRNA molecule in a polymer matrix or gel.

Pharmaceutical compositions of this disclosure suitable for parenteral administration comprise one or more siRNA molecules of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain, for example, sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions of the disclosure may also contain adjuvants such as preservatives, wetting agents, emulsifying agents and dispersing agents. Prevention of the action of microorganisms upon the subject compounds may be ensured, for example, by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about, for example, by the inclusion of agents which delay absorption such as aluminum monostearate and gelatin.

In some embodiments, in order to prolong the effect of a drug, it is desirable to slow the absorption of the drug, for example from subcutaneous or intramuscular injection. This may be accomplished, for example, by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

In some embodiments, the administration is via a depot injection. Injectable depot forms can be made by forming microencapsule matrices of the subject siRNA molecules in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations can also be prepared, for example, by entrapping the drug in liposomes or microemulsions which are compatible with body tissue.

Depot injection may release the siRNA in a consistent way over a prolonged time period. Thus, a depot injection may reduce the frequency of dosing needed to obtain a desired effect, e.g., a desired inhibition of HSD17B13, or a therapeutic or prophylactic effect. A depot injection may also provide more consistent serum concentrations. Depot injections may include, for example, subcutaneous injections or intramuscular injections. In some embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used, for example, for intravenous, subcutaneous, arterial, or epidural infusions. In some embodiments, the infusion pump is a subcutaneous infusion pump. In other embodiments, the pump is a surgically implanted pump that delivers the siRNA to the subject.

In some embodiments, the pharmaceutical compositions of the disclosure are packaged with or stored within a device for administration. Devices for injectable formulations include, but are not limited to, injection ports, pre-filled syringes, auto injectors, injection pumps, on-body injectors, and injection pens. Devices for aerosolized or powder formulations include, but are not limited to, inhalers, insufflators, aspirators, and the like. Thus, the present disclosure includes administration devices comprising a pharmaceutical composition of the disclosure for treating or preventing one or more of the disorders described herein.

The mode of administration may be chosen, for example, based upon whether local or systemic treatment is desired and based upon the area to be treated. The route and site of administration may be chosen, for example, to enhance targeting.

Regardless of the route of administration selected, the siRNA molecules of the present disclosure, which may be used in a suitable hydrated form, and/or the pharmaceutical compositions of the present disclosure, may be formulated into pharmaceutically-acceptable dosage forms by methods known to those of skill in the art. Methods for the formulation of pharmaceutical compositions depend on a number of criteria, including, but not limited to, route of administration, type and extent of disease or disorder to be treated, and/or dose to be administered. In some embodiments, the pharmaceutical compositions are formulated based on the intended route of delivery. The preparation of the pharmaceutical compositions can be carried out in a known manner. For this purpose, one or more compounds, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage.

The pharmaceutical compositions may conveniently be presented in unit dosage form and may be prepared by any methods known in the art of pharmacy. The amount of active ingredient which can be combined with a carrier material to produce a single dosage form will vary depending upon the host being treated and the particular mode of administration, for example, as described below. The amount of active ingredient which can be combined with a carrier material to produce a single dosage form will generally be, for example, that amount of the siRNA molecule which produces a therapeutic effect. In some embodiments, for example, out of one hundred percent, this amount will range from about 0.1 percent to about ninety-nine percent of active ingredient, or from about 5 percent to about 70 percent, or from about 10 percent to about 30 percent.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient. For example, the siRNA molecules in the pharmaceutical compositions of the disclosure may be administered in dosages sufficient to downregulate the expression of a HSD17B13 gene.

The siRNA molecules and pharmaceutical compositions of the present disclosure may be used to treat a disease in a subject in need thereof, for example in the methods described below.

### Dosages

The dosage amount and/or regimen utilizing a siRNA molecule of the disclosure may be selected in accordance with a variety of factors including, for example, the activity of the particular siRNA molecule of the present disclosure employed, or the salt thereof; the severity of the condition to be treated; the route of administration; the time of administration; the rate of excretion or metabolism of the particular siRNA molecule being employed; the rate and extent of absorption; the duration of the treatment; other drugs, compounds and/or materials used in combination with the particular siRNA molecule employed; the type, species, age, sex, weight, condition, general health and prior medical history of the patient being treated; the renal and hepatic function of the patient; and like factors well known in the medical arts. A consideration of these factors is well within the purview of the ordinarily skilled clinician for the purpose of determining a therapeutically effective amount.

In some embodiments, a suitable daily dose of a siRNA molecule of the disclosure is, for example, the amount of the siRNA molecule that is the lowest dose effective to produce a therapeutic effect. For example, a physician or veterinarian could start doses of the siRNA molecules of the disclosure employed in a pharmaceutical composition at levels lower than that required in order to achieve the desired therapeutic effect and gradually increase the dosage until the desired effect is achieved. Such an effective dose may depend, for example, upon the factors described above. In some embodiments, the siRNA molecules of the disclosure may be administered in dosages sufficient to downregulate or inhibit expression of a HSD17B13 gene.

In some embodiments, the siRNA molecule is administered at about 0.01 mg/kg to about 200 mg/kg, or at about 0.1 mg/kg to about 100 mg/kg, or at about 0.5 mg/kg to about 50 mg/kg. In some embodiments, the siRNA molecule is administered at about 1 mg/kg to about 40 mg/kg, or at about 1 mg/kg to about 30 mg/kg, or at about 1 mg/kg to about 20 mg/kg, or at about 1 mg/kg to about 15 mg/kg, or at about 1 mg/kg to about 10 mg/kg. In some embodiments, the siRNA molecule is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.85, 0.90, 0.95, or 1 mg/kg. In some embodiments, the siRNA molecule is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the siRNA molecule is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the siRNA molecule is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

In some embodiments, treatment of a subject with a therapeutically effective amount of a siRNA molecule of the disclosure can include a single treatment or a series of treatments. In some embodiments, the siRNA molecule is administered as a single dose or may be divided into multiple doses. In some embodiments, the effective daily dose of the siRNA molecule may be administered as two, three, four, five, six, seven, eight, nine, ten or more doses or sub-doses administered separately at appropriate intervals throughout the day, optionally, in unit dosage forms.

In some embodiments, the siRNA molecule is administered once daily. In some embodiments, the siRNA molecule is administered once weekly. In some embodiments, the siRNA molecule is administered at least 1, 2, 3, 4, 5, 6, 7, 8, 9, 10, 11, 12, 13, 14, or 15 times per day. In some embodiments, the siRNA molecule is administered at least 1, 2, 3, 4, 5, 6, 7, 8, 9, 10, 11, 12, 13, 14, 15, 16, 17, 18, 19, 20, or 21 times a week. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, the siRNA molecule is administered once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the siRNA molecule is administered every 3 days. In some embodiments, the siRNA molecule is administered once every 1, 2, 3, 4, 5, 6, 7, 8, 9, 10, 11, 12, 13, 14, or 15 weeks. In some embodiments, the siRNA molecule is administered once a month. In some embodiments, the siRNA molecule is administered once every 1, 2, 3, 4, 5, 6, 7, 8, 9, 10, 11, 12, 13, 14, or 15 months.

In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the siRNA molecule is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the siRNA molecule is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the siRNA molecule is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the siRNA molecule is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, a repeat-dose regimen may include administration of a therapeutically effective amount of siRNA on a regular basis, such as every other day, once weekly, once per quarter (i.e., about every 3 months), or once a year. In some embodiments, the dosage amount and/or frequency may be decreased after an initial treatment period. In some embodiments, when the siRNA molecules described herein are co-administered with another active agent, the therapeutically effective amount may be less than when the siRNA molecule is used alone.

### Methods and Uses

Disclosed herein are also methods of treating a HSD17B13-associated disease in a subject in need thereof, comprising administering to the subject any of the siRNA molecules and/or pharmaceutical compositions comprising a siRNA molecule disclosed herein. In an embodiment, the HSD17B13-associated disease is a liver disease.

When the siRNA molecules of the present disclosure are administered as pharmaceuticals, to humans and animals, they can be given per se or as a pharmaceutical composition as described above containing, for example, 0.1 to 99% (more preferably, 10 to 30%) of siRNA molecule in combination with a pharmaceutically acceptable carrier.

In some embodiments, a method of treating a disease in a subject in need thereof comprises administering to the subject an amount of any of the siRNA molecules disclosed herein. In an embodiment, the amount is a therapeutically effective amount. In some embodiments, a method of treating a disease in a subject in need thereof comprises administering to the subject an amount of any of the pharmaceutical compositions disclosed herein. In an embodiment, the amount is a therapeutically effective amount.

In some embodiments, a method of treating a disease in a subject in need thereof comprises administering to the subject any of the siRNA molecules or pharmaceutical compositions disclosed herein in combination with an additional active agent. In some embodiments, the additional active agent is a liver disease treatment agent. In an embodiment, the amount of the siRNA molecule is a therapeutically effective amount. In an embodiment, the amount of the additional active agent is a therapeutically effective amount.

In some embodiments, the siRNA molecule and the liver disease treatment agent are administered separately. In some embodiments, the siRNA molecule or pharmaceutical composition and the liver disease treatment agent are administered concurrently. In some embodiments, the siRNA molecule or pharmaceutical composition and the liver disease treatment agent are administered sequentially. In some embodiments, the siRNA molecule or pharmaceutical composition is administered prior to administering the liver disease treatment agent. In some embodiments, the siRNA molecule or pharmaceutical composition is administered after administering the liver disease treatment agent. In some embodiments, the pharmaceutical composition comprises the siRNA and the liver disease treatment agent.

Also disclosed herein are methods of reducing the expression level of HSD17B13 in a subject in need thereof comprising administering to the subject an amount of a siRNA molecule or pharmaceutical composition according to the disclosure. In an embodiment, the amount of the additional active agent is a therapeutically effective amount. In some embodiments, the method of reducing the expression level of HSD17B13 in a subject in need thereof comprising administering to the subject an amount of a siRNA molecule or pharmaceutical composition according to the disclosure reduces the expression level of HSD17B13 in hepatocytes in the subject following administration of the siRNA molecule or pharmaceutical composition as compared to the HSD17B13 expression level in a patient not receiving the siRNA or pharmaceutical composition.

Also disclosed herein are methods of preventing at least one symptom of a liver disease in a subject in need thereof comprising administering to the subject an amount of any of the siRNA molecules or pharmaceutical compositions of the disclosure, thereby preventing at least one symptom of a liver disease in the subject. In an embodiment, the amount of the additional active agent is a therapeutically effective amount.

In another aspect, disclosed herein are uses of any of the siRNA molecules or pharmaceutical compositions of the disclosure in the manufacture of a medicament for treating a liver disease. In some embodiments, the present disclosure provides use of a siRNA molecule of the disclosure or pharmaceutical composition comprising an siRNA of the disclosure that targets a HSD17B13 gene in a cell of a mammal in the manufacture of a medicament for inhibiting expression of the HSD17B13 gene in the mammal.

The methods and uses disclosed herein include administering to a mammal, e.g., a human, a pharmaceutical composition comprising a siRNA molecule that targets a HSD17B13 gene in a cell of the mammal and maintaining for a time sufficient to obtain degradation of the mRNA transcript of the HSD17B13 gene, thereby inhibiting expression of the HSD17B13 gene in the mammal.

The patient or subject of the described methods may be a mammal, and it includes humans and non-human mammals. In some embodiments, the subject is a human, such as an adult human, human teenager, human child, human toddler, or human infant.

The siRNA molecules and/or pharmaceutical compositions of the disclosure can be administered in the disclosed methods and uses by any administration route known in the art, including those described above such as, for example, subcutaneous, intravenous, oral, intraperitoneal, or parenteral routes, including, e.g., intracranial (e.g., intraventricular, intraparenchymal and intrathecal), intramuscular, transdermal, airway (aerosol), nasal, rectal, and topical (including buccal and sublingual) administration.

The siRNA molecules and/or pharmaceutical compositions of the disclosure can be administered in the disclosed methods and uses in any of the of dosages or dosage regimens described above.

### HSD17B13-Associated Diseases

Any of the siRNAs and/or pharmaceutical compositions and/or methods and/or uses disclosed herein may be used to treat a disease, disorder, and/or condition. In some embodiments, the disease, disorder, and/or condition is associated with HSD17B13 expression or activity. In some embodiments, the disease, disorder, and/or condition is a liver disease. As used herein, the term “HSD17B13-associated disease” includes a disease, disorder, or condition that would benefit from a downregulation in HSD17B13 gene expression, replication or activity. Non-limiting examples of HSD17B13-associated diseases include, but are not limited to, fatty liver (steatosis), nonalcoholic steatohepatitis (NASH), cirrhosis of the liver, accumulation of fat in the liver, inflammation of the liver, hepatocellular necrosis, liver fibrosis, obesity, hepatocellular carcinoma (HCC), or nonalcoholic fatty liver disease (NAFLD). In an embodiment, the HSD17B13-associated disease is NAFLD. In an embodiments, the HSD17B13-associated disease is NASH. In an embodiment, the HSD17B13-associated disease is fatty liver (steatosis). In an embodiment, the HSD17B13-associated disease is NAFLD. In an embodiment, the HSD17B13-associated disease is HCC.

### Combination Therapies

Any of the siRNAs or pharmaceutical compositions disclosed herein may be combined with one or more additional active agents in a pharmaceutical composition or in any method according to the disclosure or for use in treating a liver disease. An additional active agent refers to an ingredient with a pharmacologically effect at a relevant dose; an additional active agent may be another siRNA according to the disclosure, a siRNA not in accordance with the disclosure, or a non-siRNA active agent.

In some embodiments, at least 2, 3, 4, 5, 6, 7, 8, 9, 10 or more siRNAs disclosed herein are combined in a combination therapy.

In some embodiments, any of the siRNAs or pharmaceutical compositions disclosed herein are combined with a liver disease treatment agent in a combination therapy. In some embodiments, the liver disease treatment agent is selected from a peroxisome proliferator-activator receptor (PPAR) agonist, farnesoid X receptor (FXR) agonist, lipid-altering agent, incretin-based therapy, PNPLA3 inhibitors, and thyroid hormone receptor (THR) modulator.

In some embodiments, any of the siRNAs or pharmaceutical compositions disclosed herein are combined with a PPAR agonist. In some embodiments, the PPAR agonist is selected from a PPARα agonist, dual PPARα/δ agonist, PPARγ agonist, and dual PPARα/γ agonist. In some embodiments, the dual PPARα agonist is a fibrate. In some embodiments, the PPARα/δ agonist is elafibranor. In some embodiments, the PPARγ agonist is a thiazolidinedione (TZD). In some embodiments, TZD is pioglitazone. In some embodiments, the dual PPARα/γ agonist is saroglitazar.

In some embodiments, any of the siRNAs or pharmaceutical compositions disclosed herein are combined with a FXR agonist. In some embodiments, the FXR agonist is selected from obeticholic acis (OCA) and TERN-1010.

In some embodiments, any of the siRNAs or pharmaceutical compositions disclosed herein are combined with a lipid-altering agent. In some embodiments, the lipid-altering agent is aramchol.

In some embodiments, any of the siRNAs or pharmaceutical compositions disclosed herein are combined with an incretin-based therapy. In some embodiments, the incretin-based therapy is a glucagon-like peptide 1 (GLP-1) receptor agonist or dipeptidyl peptidase 4 (DPP-4) inhibitor. In some embodiments, the GLP-1 receptor agonist is exenatide or liraglutide. In some embodiments, the DPP-4 inhibitor is sitagliptin or vildapliptin.

In some embodiments, any of the siRNAs or pharmaceutical compositions disclosed herein are combined with a THR modulator. In some embodiments, the THR modulator is selected from a THR-beta modulator and thyroid hormone analogue. Exemplary THR modulators are described in Jakobsson, et al., Drugs, 2017, 77 (15): 1613-1621, Saponaro, et al., Front Med (Lausanne), 2020, 7:331, and Kowalik, et al., Front Endocrinol, 2018, 9:382, which are incorporated by reference in their entireties. In some embodiments, the THR-beta modulator is a THR-beta agonist. In some embodiments, the THR-beta agonist is selected from is selected from KB141, sobetirome, Sob-AM2, eprotirome, VK2809, resmetirom, MB07344, IS25, TG68, GC-24 and any one of the compounds disclosed in U.S. Pat. No. 11,091,467, which is incorporated in its entirety herein. In some embodiments, the thyroid hormone analogue is selected from L-94901 and CG-23425.

Generally, the liver disease treatment agent may be used in any combination with the siRNA molecules of the disclosure in a single dosage formulation (e.g., a fixed dose drug combination), or in one or more separate dosage formulations which allows for concurrent or sequential administration of the active agents (co-administration of the separate active agents) to subjects. In some embodiments, the siRNA and the liver disease treatment agent are administered concurrently. In some embodiments, the siRNA and the liver disease treatment agent are administered sequentially. In some embodiments, the siRNA is administered prior to administering the liver disease treatment agent. In some embodiments, the siRNA is administered after administering the liver disease treatment agent. The sequence and frequency in which the siRNA and the liver disease treatment agent are administered can vary. In some embodiments, the siRNA and the liver disease treatment agent are in separate containers. In some embodiments, the siRNA and the liver disease treatment agent are in the same container. In some embodiments, the pharmaceutical composition comprises the siRNA and the liver disease treatment agent. The siRNA and the liver disease treatment agent can be administered by the same route of administration or by different routes of administration.

Still other embodiments of the disclosure have the following features.

The present technology provides a short interfering nucleic acid (siNA) molecule. The siNA may be single-stranded. Alternatively, the siNA may be double-stranded (ds-siNA) molecules. In any embodiment, the nucleotides may be modified nucleotides, non-modified nucleotides, or any combination thereof. The nucleotides may be ribonucleotides, deoxyribonucleotides, or any combination thereof. The siNA may comprise at least 5 nucleotides. The siNA molecules described herein may comprise modified nucleotides selected from 2′-O-methyl nucleotides and 2′-fluoro nucleotides.

In any embodiment, the first nucleotide sequence may include a nucleotide sequence of any one of SEQ ID Nos: 1-100, 201-230, 262-287, 314, or 315. In any embodiment, the second nucleotide sequence may include a nucleotide sequence of any one of SEQ ID NOs: 101-200, 231-260, or 288-313.

In any embodiment, the siNA may reduce or inhibit the production of a hydroxysteroid dehydrogenase. In any embodiment, the siNA may target a hydroxysteroid 17-beta dehydrogenase 13 (HSD17B13) gene.

In any embodiment, the siNA molecules described herein may comprise 1, 2, 3, 4, 5, 6, 7, 8, 9, or 10 or more phosphorothioate internucleoside linkages. In any embodiment, the siNA molecules described herein may comprise 1, 2, 3, 4, 5, 6, 7, 8, 9, or 10 or more mesyl phosphoroamidate internucleoside linkage(s).

In any embodiment, the siNA molecules described herein may comprise a phosphorylation blocker. In any embodiment, the siNA molecules described herein may comprise a 5′-stabilized end cap. In any embodiment, the siNA molecules described herein may comprise a galactosamine. In any embodiment, the siNA molecules described herein may comprise a conjugated moeity. In any embodiment, the siNA molecules described herein may comprise a destabilizing nucleotide. In any embodiment, the siNA molecules described herein may comprise a modified nucleotide. In any embodiment, the siNA molecules described herein may comprise a thermally destabilizing nucleotide.

In any embodiment, the siNA molecules described herein may comprise one or more blunt ends. In any embodiment, the siNA molecules described herein may comprise one or more overhangs.

In one aspect, the siNA molecule comprises: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

In another aspect, the present technology also provides a molecule represented by Formula (VIII):

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O-methyl nucleotide or a nucleotide comprising a 5′-stabilized end cap or a phosphorylation blocker; B is a 2′-fluoro nucleotide; C represents overhanging nucleotides and is a 2′-O-methyl nucleotide, deoxy nucleotide, or uracil; n¹=1-6 nucleotides in length; each n², n⁶, n⁸, q³, q⁵, q⁷, q⁹, q¹¹, and q¹² is independently 0-1 nucleotides in length; each n³ and n⁴ is independently 1-3 nucleotides in length; n⁵ is 1-10 nucleotides in length; n⁷ is 0-4 nucleotides in length; each n⁹, q¹, and q² is independently 0-2 nucleotides in length; q⁴ is 0-3 nucleotides in length; q⁶ is 0-5 nucleotides in length; q⁸ is 2-7 nucleotides in length; and q¹⁰ is 2-11 nucleotides in length.

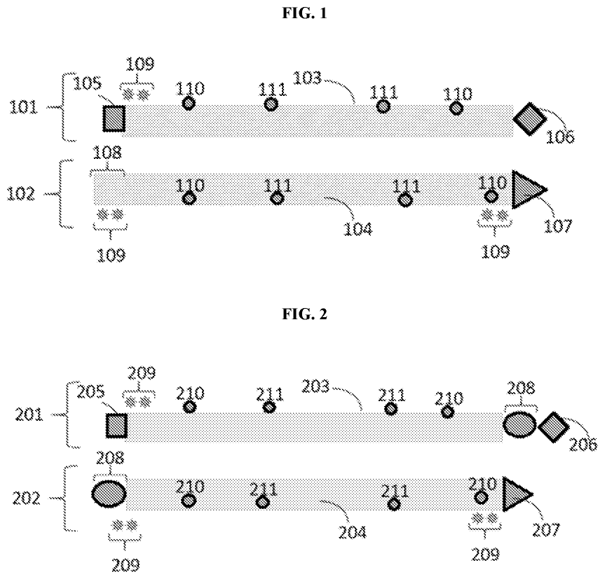

An exemplary siNA molecule of the present disclosure is shown in FIG. 1. As shown in FIG. 1, an exemplary siNA molecule comprises a sense strand (101) and an antisense strand (102). The sense strand (101) may comprise a first oligonucleotide sequence (103). The first oligonucleotide sequence (103) may comprise one or more phosphorothioate internucleoside linkages (109). The phosphorothioate internucleoside linkage (109) may be between the nucleotides at the 5′ or 3′ terminal end of the first oligonucleotide sequence (103). The phosphorothioate internucleoside linkage (109) may be between the first three nucleotides from the 5′ end of the first oligonucleotide sequence (103). The first oligonucleotide sequence (103) may comprise one or more 2′-fluoro nucleotides (110). The first oligonucleotide sequence (103) may comprise one or more 2′-O methyl nucleotides (111). The first oligonucleotide sequence (103) may comprise 15 or more modified nucleotides independently selected from 2′-fluoro nucleotides (110) and 2′-O methyl nucleotides (111). The sense strand (101) may further comprise a phosphorylation blocker (105). The sense strand (101) may further comprise a galactosamine (106). The antisense strand (102) may comprise a second oligonucleotide sequence (104). The second oligonucleotide sequence (104) may comprise one or more phophorothioate internucleoside linkages (109). The phosphorothioate internucleoside linkage (109) may be between the nucleotides at the 5′ or 3′ terminal end of the second oligonucleotide sequence (104). The phosphorothioate internucleoside linkage (109) may be between the first three nucleotides from the 5′ end of the second oligonucleotide sequence (104). The phosphorothioate internucleoside linkage (109) may be between the first three nucleotides from the 3′ end of the second oligonucleotide sequence (104). The second oligonucleotide sequence (104) may comprise one or more 2′-fluoro nucleotides (110). The second oligonucleotide sequence (104) may comprise one or more 2′-O methyl nucleotides (111). The second oligonucleotide sequence (104) may comprise 15 or more modified nucleotides independently selected from 2′-fluoro nucleotides (110) and 2′-O methyl nucleotides (111). The antisense strand (102) may further comprise a 5′-stabilized end cap (107). The siNA may further comprise one or more blunt ends. Alternatively, or additionally, one end of the siNA may comprise an overhang (108). The overhang (108) may be part of the sense strand (101). The overhang (108) may be part of the antisense strand (102). The overhang (108) may be distinct from the first nucleotide sequence (103). The overhang (108) may be distinct from the second nucleotide sequence (104). The overhang (108) may be part of the first nucleotide sequence (103). The overhang (108) may be part of the second nucleotide sequence (104). The overhang (108) may comprise 1 or more nucleotides. The overhang (108) may comprise 1 or more deoxyribonucleotides. The overhang (108) may comprise 1 or more modified nucleotides. The overhang (108) may comprise 1 or more modified ribonucleotides. The sense strand (101) may be shorter than the antisense strand (102). The sense strand (101) may be the same length as the antisense strand (102). The sense strand (101) may be longer than the antisense strand (102).

An exemplary siNA molecule of the present disclosure is shown in FIG. 2. As shown in FIG. 2, an exemplary siNA molecule comprises a sense strand (201) and an antisense strand (202). The sense strand (201) may comprise a first oligonucleotide sequence (203). The first oligonucleotide sequence (203) may comprise one or more phophorothioate internucleoside linkages (209). The phosphorothioate internucleoside linkage (209) may be between the nucleotides at the 5′ or 3′ terminal end of the first oligonucleotide sequence (203). The phosphorothioate internucleoside linkage (209) may be between the first three nucleotides from the 5′ end of the first oligonucleotide sequence (203). The first oligonucleotide sequence (203) may comprise one or more 2′-fluoro nucleotides (210). The first oligonucleotide sequence (203) may comprise one or more 2′-O methyl nucleotides (211). The first oligonucleotide sequence (203) may comprise 15 or more modified nucleotides independently selected from 2′-fluoro nucleotides (210) and 2′-O methyl nucleotides (211). The sense strand (201) may further comprise a phosphorylation blocker (205). The sense strand (201) may further comprise a galactosamine (206). The antisense strand (202) may comprise a second oligonucleotide sequence (204). The second oligonucleotide sequence (204) may comprise one or more phophorothioate internucleoside linkages (209). The phosphorothioate internucleoside linkage (209) may be between the nucleotides at the 5′ or 3′ terminal end of the second oligonucleotide sequence (204). The phosphorothioate internucleoside linkage (209) may be between the first three nucleotides from the 5′ end of the second oligonucleotide sequence (204). The phosphorothioate internucleoside linkage (209) may be between the first three nucleotides from the 3′ end of the second oligonucleotide sequence (204). The second oligonucleotide sequence (204) may comprise one or more 2′-fluoro nucleotides (210). The second oligonucleotide sequence (204) may comprise one or more 2′-O methyl nucleotides (211). The second oligonucleotide sequence (204) may comprise 15 or more modified nucleotides independently selected from 2′-fluoro nucleotides (210) and 2′-O methyl nucleotides (211). The antisense strand (202) may further comprise a 5′-stabilized end cap (207). The siNA may further comprise one or more overhangs (208). The overhang (208) may be part of the sense strand (201). The overhang (208) may be part of the antisense strand. (202). The overhang (208) may be distinct from the first nucleotide sequence (203). The overhang (208) may be distinct from the second nucleotide sequence (204). The overhang (208) may be part of the first nucleotide sequence (203). The overhang (208) may be part of the second nucleotide sequence (204). The overhang (208) may be adjacent to the 3′ end of the first nucleotide sequence (203). The overhang (208) may be adjacent to the 5′ end of the first nucleotide sequence (203). The overhang (208) may be adjacent to the 3′ end of the second nucleotide sequence (204). The overhang (208) may be adjacent to the 5′ end of the second nucleotide sequence (204). The overhang (208) may comprise 1 or more nucleotides. The overhang (208) may comprise 1 or more deoxyribonucleotides. The overhang (208) may comprise a TT sequence. The overhang (208) may comprise 1 or more modified nucleotides. The overhang (208) may comprise 1 or more modified nucleotides disclosed herein (e.g., 2-fluoro nucleotide, 2′-O methyl nucleotide, 2′-fluoro nucleotide mimic, 2′-O methyl nucleotide mimic, or a nucleotide comprising a modified nucleobase). The overhang (208) may comprise 1 or more modified ribonucleotides. The sense strand (201) may be shorter than the antisense strand (202). The sense strand (201) may be the same length as the antisense strand (202). The sense strand (201) may be longer than the antisense strand (202).

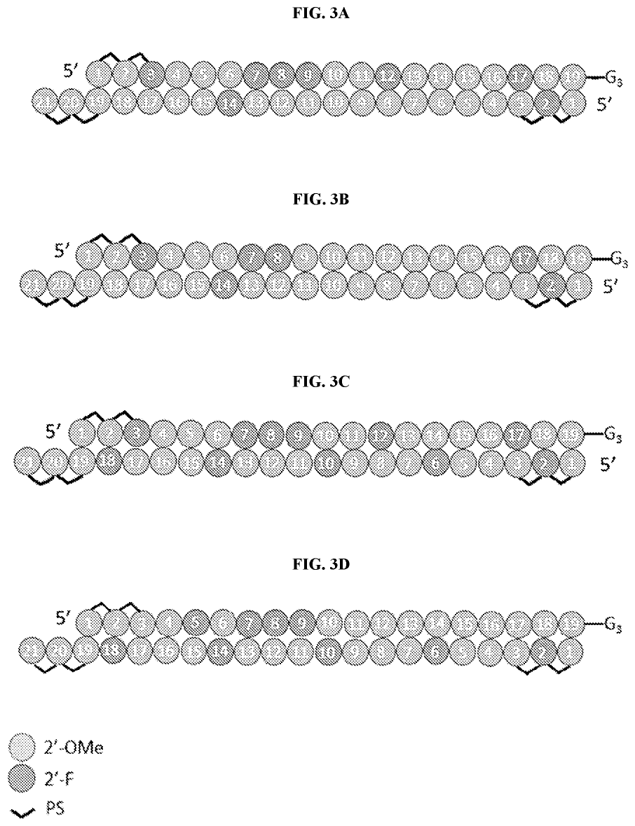

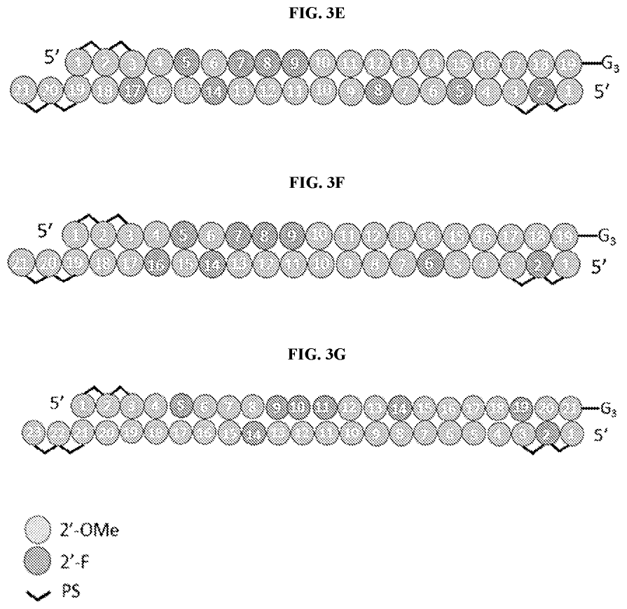

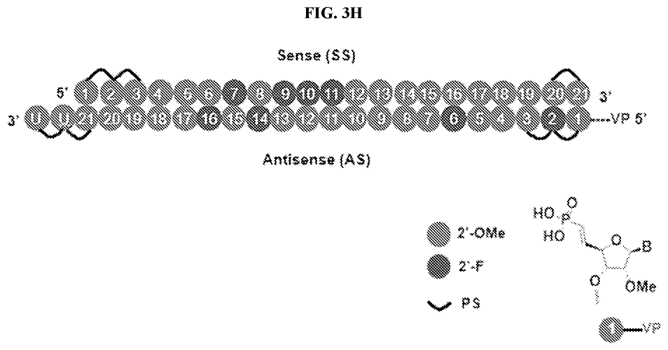

FIGS. 3A-3H depict exemplary ds-siNA modification patterns. As shown in FIGS. 3A-3H, an exemplary ds-siNA molecule may have the following formula:

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O methyl nucleotide or a nucleotide comprising a 5′ stabilized end cap or phosphorylation blocker; B is a 2′-fluoro nucleotide; C represents overhanging nucleotides and is a 2′-O methyl nucleotide, deoxy nucleotide, or uracil; n¹=1-6 nucleotides in length; each n², n⁶, n⁸, q³, q⁵, q⁷, q⁹, q¹¹, and q¹² is independently 0-1 nucleotides in length; each n³ and n⁴ is independently 1-3 nucleotides in length; n⁵ is 1-10 nucleotides in length; n⁷ is 0-4 nucleotides in length; each n⁹, q¹, and q² is independently 0-2 nucleotides in length; q⁴ is 0-3 nucleotides in length; q⁶ is 0-5 nucleotides in length; q⁸ is 2-7 nucleotides in length; and q¹⁰ is 2-11 nucleotides in length.

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The 5′-stabilizing end cap may be attached to the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker.

An exemplary ds-siNA molecule may have the following formula:

5′-A₂₋₆B₁A₁₋₃B₂₋₃A₂₋₁₀B₀₋₁A₀₋₄B₀₋₁A₀₋₂-3′
3′-C₂A₀₋₂B₀₋₁A₀₋₃B₀₋₁A₀₋₅B₀₋₁A₂₋₇B₁A₂₋₁₁B₁A₁-5′

wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O methyl nucleotide or a nucleotide comprising a 5′ stabilized end cap or phosphorylation blocker; B is a 2′-fluoro nucleotide; C represents overhanging nucleotides and is a 2′-O methyl nucleotide, deoxy nucleotide, or uracil.

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The vinyl phosphonate may be a deuterated vinyl phosphonate. The deuterated vinyl phosphonate may be a mono-deuterated vinyl phosphonate. The deuterated vinyl phosphonate may be a mono-di-deuterated vinyl phosphonate. The 5′-stabilizing end cap may be attached to the 5′ end of the antisense strand. The 5′-stabilizing end cap may be attached to the 3′ end of the antisense strand. The 5′-stabilizing end cap may be attached to the 5′ end of the sense strand. The 5′-stabilizing end cap may be attached to the 3′ end of the sense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker.

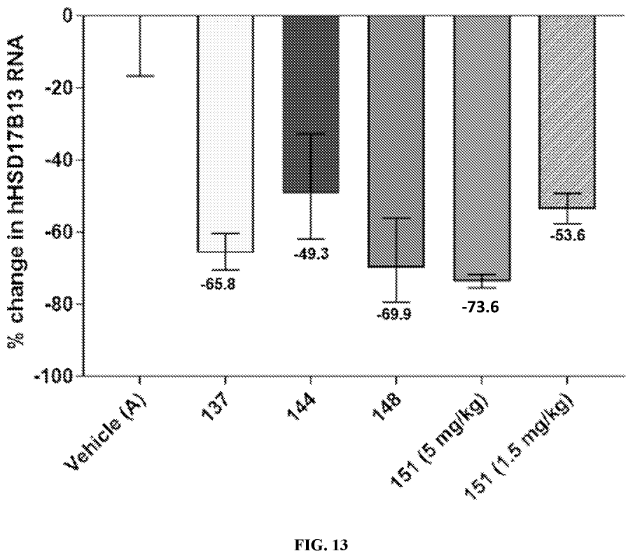

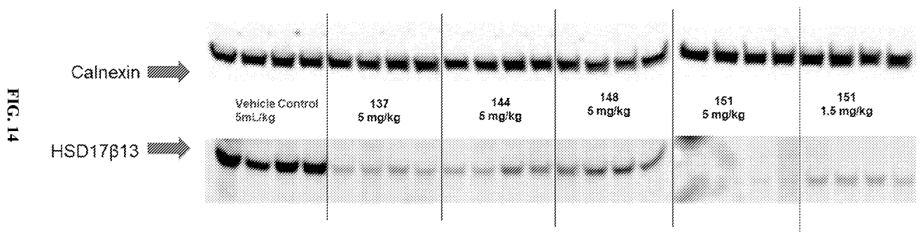

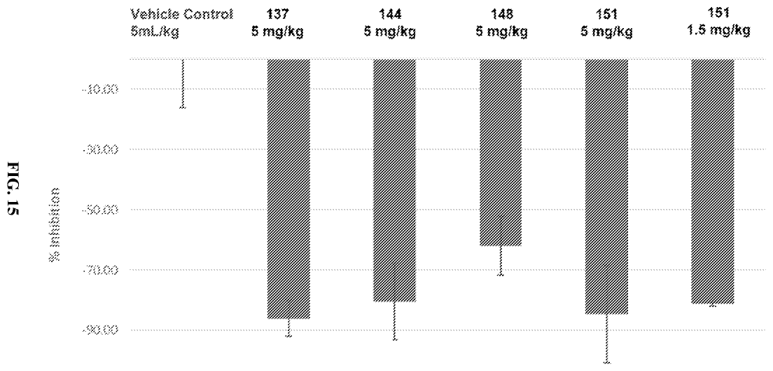

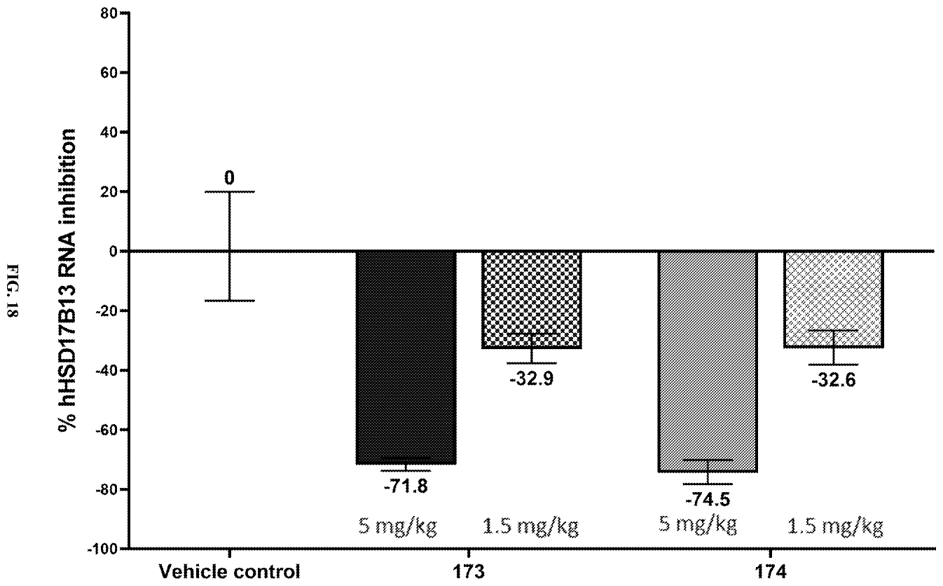

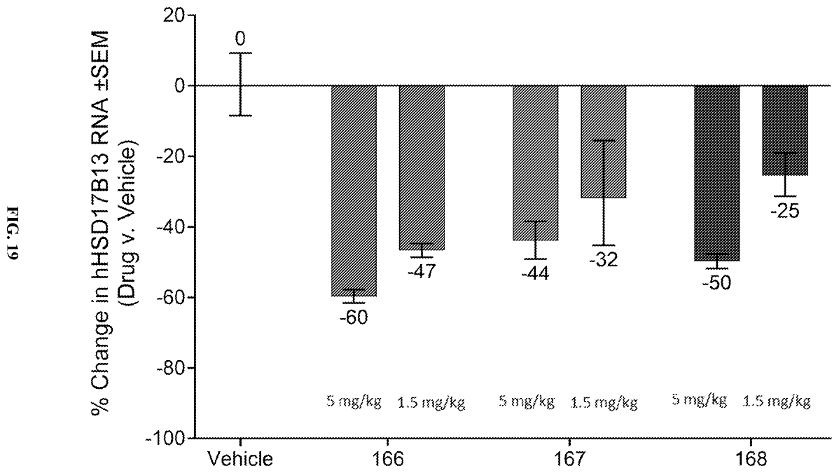

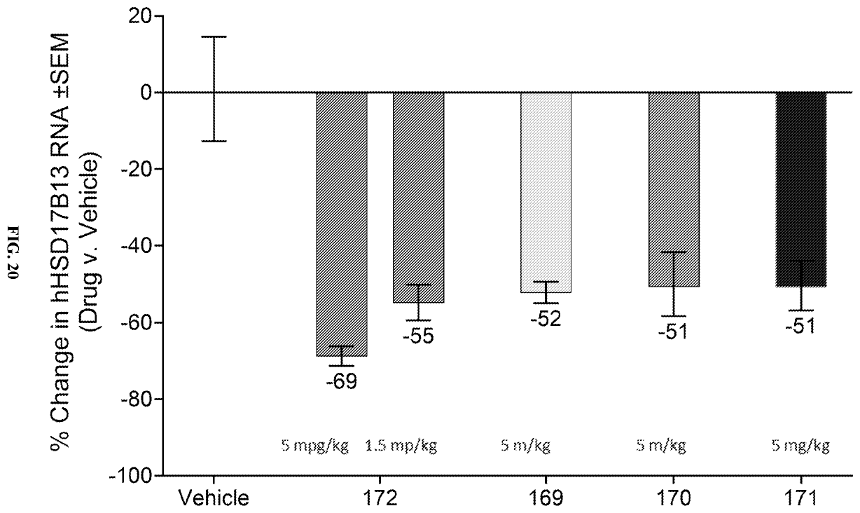

The exemplary ds-siNA shown in FIGS. 3A-3H comprise (i) a sense strand comprising 19-21 nucleotides; and (ii) an antisense strand comprising 21-23 nucleotides. The ds-siNA may further comprise (iii) a conjugated moiety, wherein the conjugated moiety is attached to the 3′ end of the antisense strand. The ds-siNA may comprise a 2 nucleotide overhang consisting of nucleotides at positions 20 and 21 from the 5′ end of the antisense strand. The ds-siNA may comprise a 2 nucleotide overhang consisting of nucleotides at positions 22 and 23 from the 5′ end of the antisense strand. The ds-siNA may further comprise 1, 2, 3, 4, 5, 6 or more phosphorothioate (ps) internucleoside linkages. At least one phosphorothioate internucleoside linkage may be between the nucleotides at positions 1 and 2 or positions 2 and 3 from the 5′ end of the sense strand. At least one phosphorothioate internucleoside linkage may be between the nucleotides at positions 1 and 2 or positions 2 and 3 from the 5′ end of the antisense strand. At least one phosphorothioate internucleoside linkage may be between the nucleotides at positions 19 and 20, positions 20 and 21, positions 21 and 22, or positions 22 and 23 from the 5′ end of the antisense strand. As shown in FIGS. 3A-3H, 4-6 nucleotides in the sense strand may be 2′-fluoro nucleotides. As shown in FIGS. 3A-3H, 2-5 nucleotides in the antisense strand may be 2′-fluoro nucleotides. As shown in FIGS. 3A-3H, 13-15 nucleotides in the sense strand may be 2′-O methyl nucleotides. As shown in FIGS. 3A-3H, 14-19 nucleotides in the antisense strand may be 2′-O methyl nucleotides. As shown in FIGS. 3A-3H, the ds-siNA does not contain a base pair between 2′-fluoro nucleotides on the sense and antisense strands. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker.

In some embodiments, the (a) a sense strand may comprise a first nucleotide sequence consisting of 17 to 23 nucleotides, wherein 2′-fluoro nucleotides are at positions 3, 7-9, 12, and 17 from the 5′ end of the first nucleotide sequence, and wherein 2′-O methyl nucleotides are at positions 1, 2, 4-6, 10, 11, and 13-16 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence. As shown in FIG. 3A, a ds-siNA may comprise (a) a sense strand consisting of 19 nucleotides, wherein 2′-fluoro nucleotides are at positions 3, 7-9, 12, and 17 from the 5′ end of the sense strand, and wherein 2′-O methyl nucleotides are at positions 1, 2, 4-6, 10, 11, 13-16, 18, and 19 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a fB, fN, f(4nh)Q, f4P, f2P, or fX nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 3, 7, 8, and 17 from the 5′ end of the first nucleotide sequence, and wherein 2′-O methyl nucleotides are at positions 1, 2, 4-6, and 9-16 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence. As shown in FIG. 3B, a ds-siNA may comprise (a) a sense strand consisting of 19 nucleotides, wherein 2′-fluoro nucleotides are at positions 3, 7, 8, and 17 from the 5′ end of the sense strand, and wherein 2′-O methyl nucleotides are at positions 1, 2, 4-6, 9-16, 18, and 19 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a fB, fN, f(4nh)Q, f4P, f2P, or fX nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 3, 7-9, and 17 from the 5′ end of the first nucleotide sequence, and wherein 2′-O methyl nucleotides are at positions 1, 2, 4-6, and 10-16 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 10, and 14 from the 5′ end of the second nucleotide sequence, and wherein 2′-O methyl nucleotides are at positions 1, 3-5, 7-9, 11-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 19-21 from the 5′ end of the second nucleotide sequence. As shown in FIG. 3C, a ds-siNA may comprise (a) a sense strand consisting of 19 nucleotides, wherein 2′-fluoro nucleotides are at positions 3, 7-9, 12 and 17 from the 5′ end of the sense strand, and wherein 2′-O methyl nucleotides are at positions 1, 2, 4-6, 10, 11, 13-16, 18, and 19 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 10, and 14 from the 5′ end of the second nucleotide sequence, and wherein 2′-O methyl nucleotides are at positions 1, 3-5, 7-9, 11-13 and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-fluoro nucleotide is at position 18 from the 5′ end of the second nucleotide sequence. In any embodiment, 2′-O methyl nucleotides are at positions 19-21 from the 5′ end of the second nucleotide sequence. As shown in FIG. 3D, a ds-siNA may comprise (a) a sense strand consisting of 19 nucleotides, wherein 2′-fluoro nucleotides are at positions 5 and 7-9 from the 5′ end of the sense strand, and wherein 2′-O methyl nucleotides are at positions 1-4, 6, and 10-19 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. The alternating 1:3 modification pattern may start at the nucleotide at any of positions 2, 6, 10, 14, and/or 18 from the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, or f2P nucleotide. In some embodiments, at least 1, 2, 3, 4 or more 2′-fluoro nucleotides on the antisense strand is a fB, fN, f(4nh)Q, f4P, or f2P nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 5, 8, 14, and 17 from the 5′ end of the first nucleotide sequence, and wherein 2′-O methyl nucleotides are at positions 1, 3, 4, 6, 7, 9-13, 15, and 16 from the 5′ end of the first nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence. As shown in FIG. 3E, a ds-siNA may comprise (a) a sense strand consisting of 19 nucleotides, wherein 2′-fluoro nucleotides are at positions 5 and 7-9 from the 5′ end of the sense strand, and wherein 2′-O methyl nucleotides are at positions 1-4, 6, and 10-19 from the 5′ end of the sense strand; (b) an antisense strand consisting of 21 nucleotides, wherein the nucleotides in the antisense strand comprise an alternating 1:2 modification pattern, and wherein 1 nucleotide is a 2′-fluoro nucleotide and 2 nucleotides are 2′-O methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:2 modification patterns on the antisense strand. The alternating 1:2 modification pattern may start at the nucleotide at any of positions 2, 5, 8, 14, and/or 17 from the 5′ end of the antisense strand. In some embodiments, the ds-siNA comprises (a) a sense strand consisting of 19 nucleotides, wherein 2′-fluoro nucleotides are at positions 5 and 7-9 from the 5′ end of the sense strand, and wherein 2′-O methyl nucleotides are at positions 1-4, 6, and 10-19 from the 5′ end of the sense strand; (b) an antisense strand consisting of 21 nucleotides, wherein 2′-fluoro nucleotides are at positions 2, 5, 8, 14, and 17 from the 5′ end of the antisense strand, and wherein 2′-O methyl nucleotides are at positions 1, 3, 4, 6, 7, 9-13, 15, 16, and 18-21 from the 5′ end of the sense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, or f2P nucleotide. In some embodiments, at least 1, 2, 3, 4 or more 2′-fluoro nucleotides on the antisense strand is a fB, fN, f(4nh)Q, f4P, or f2P nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 14, and 16 from the 5′ end of the second nucleotide sequence, and wherein 2′-O methyl nucleotides are at positions 1, 3-5, 7-13, 15, and 17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence. As shown in FIG. 3F, a ds-siNA may comprise (a) a sense strand consisting of 19 nucleotides, wherein 2′-fluoro nucleotides are at positions 5 and 7-9 from the 5′ end of the sense strand, and wherein 2′-O methyl nucleotides are at positions 1-4, 6, and 10-19 from the 5′ end of the sense strand; (b) an antisense strand consisting of 21 nucleotides, wherein 2′-fluoro nucleotides are at positions 2, 6, 14, and 16 from the 5′ end of the antisense strand, and wherein 2′-O methyl nucleotides are at positions 1, 3-5, 7-13, 15, and 17-21 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, at least 1, 2, 3, 4 or more 2′-fluoro nucleotides on the sense strand or antisense strand is a fB, fN, f(4nh)Q, f4P, or f2P nucleotide. In some embodiments, at least 1, 2, 3, 4 or more 2′-fluoro nucleotides on the sense strand or antisense strand is a f4P nucleotide. In some embodiments, at least 1, 2, 3, or 4 of the 2′-fluoro-nucleotides at positions 2, 6, 14, and 16 from the 5′ end of the antisense strand is a f4P nucleotide. In some embodiments, at least one of the 2′-fluoro-nucleotides at positions 2, 6, 14, and 16 from the 5′ end of the antisense strand is a f4P nucleotide. In some embodiments, at least two of the 2′-fluoro-nucleotides at positions 2, 6, 14, and 16 from the 5′ end of the antisense strand is a f4P nucleotide. In some embodiments, less than or equal to 3 of the 2′-fluoro-nucleotides at positions 2, 6, 14, and 16 from the 5′ end of the antisense strand is a f4P nucleotide. In some embodiments, less than or equal to 2 of the 2′-fluoro-nucleotides at positions 2, 6, 14, and 16 from the 5′ end of the antisense strand is a f4P nucleotide. In some embodiments, the 2′-fluoro-nucleotide at position 2 from the 5′ end of the antisense strand is a f4P nucleotide. In some embodiments, the 2′-fluoro-nucleotide at position 6 from the 5′ end of the antisense strand is a f4P nucleotide. In some embodiments, the 2′-fluoro-nucleotide at position 14 from the 5′ end of the antisense strand is a f4P nucleotide. In some embodiments, the 2′-fluoro-nucleotide at position 16 from the 5′ end of the antisense strand is a f4P nucleotide. In some embodiments, at least 1, 2, 3, 4 or more 2′-fluoro nucleotides on the sense strand or antisense strand is a f2P nucleotide. In some embodiments, at least 1, 2, 3, or 4 of the 2′-fluoro-nucleotides at positions 2, 6, 14, and 16 from the 5′ end of the antisense strand is a f2P nucleotide. In some embodiments, at least one of the 2′-fluoro-nucleotides at positions 2, 6, 14, and 16 from the 5′ end of the antisense strand is a f2P nucleotide. In some embodiments, at least two of the 2′-fluoro-nucleotides at positions 2, 6, 14, and 16 from the 5′ end of the antisense strand is a f2P nucleotide. In some embodiments, less than or equal to 3 of the 2′-fluoro-nucleotides at positions 2, 6, 14, and 16 from the 5′ end of the antisense strand is a f2P nucleotide. In some embodiments, less than or equal to 2 of the 2′-fluoro-nucleotides at positions 2, 6, 14, and 16 from the 5′ end of the antisense strand is a f2P nucleotide. In some embodiments, the 2′-fluoro-nucleotide at position 2 from the 5′ end of the antisense strand is a f2P nucleotide. In some embodiments, the 2′-fluoro-nucleotide at position 6 from the 5′ end of the antisense strand is a f2P nucleotide. In some embodiments, the 2′-fluoro-nucleotide at position 14 from the 5′ end of the antisense strand is a f2P nucleotide. In some embodiments, the 2′-fluoro-nucleotide at position 16 from the 5′ end of the antisense strand is a f2P nucleotide. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, or f2P nucleotide. In some embodiments, at least 1, 2, 3, 4 or more 2′-fluoro nucleotides on the antisense strand is a fB, fN, f(4nh)Q, f4P, or f2P nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 5, 9-11, and 14 from the 5′ end of the first nucleotide sequence, and wherein 2′-O methyl nucleotides are at positions 1-4, 6-8, 12, 13, and 15-17 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 21 nucleotides. In any embodiment, 2′-O methyl nucleotides are at positions 18, 20, and 21 from the 5′ end of the first nucleotide sequence. In any embodiment, 2′-fluoro nucleotide is at position 19 from the 5′ end of the first nucleotide sequence. In any embodiment, the second nucleotide sequence consists of 23 nucleotides. In any embodiment, 2′-O methyl nucleotides are at positions 18-23 from the 5′ end of the second nucleotide sequence. As shown in FIG. 3G, a ds-siNA may comprise (a) a sense strand consisting of 21 nucleotides, wherein 2′-fluoro nucleotides are at positions 5, 9-11, 14, and 19 from the 5′ end of the sense strand, and wherein 2′-O methyl nucleotides are at positions 1-4, 6-8, 12, 13, 15-18, 20, and 21 from the 5′ end of the sense strand; and (b) an antisense strand consisting of 23 nucleotides, wherein 2′-flouro nucleodies are at positions 2 and 14 from the 5′ end of the antisense strand, and wherein 2′-O methyl nucleotides are at positions 1, 3-13, and 15-23 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, or f2P nucleotide. In some embodiments, at least 1, 2, 3, 4 or more 2′-fluoro nucleotides on the antisense strand is a fB, fN, f(4nh)Q, f4P, or f2P nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

In some embodiments, the (a) a sense strand comprising a first nucleotide sequence consisting of 17 to 23 nucleotides, wherein 2′-fluoro nucleotides are at positions 7 and 9-11 from the 5′ end of the first nucleotide sequence, and wherein 2′-O methyl nucleotides are at positions 1-6, 8, and 12-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 14, and 16 from the 5′ end of the second nucleotide sequence, and wherein 2′-O methyl nucleotides are at positions 1, 3-5, 7-13, 15, and 17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 21 nucleotides. In any embodiment, 2′-O methyl nucleotides are at positions 18-21 from the 5′ end of the first nucleotide sequence. In any embodiment, the second nucleotide sequence consists of 23 nucleotides. In any embodiment, 2′-O methyl nucleotides are at positions 18-21 from the 5′ end of the second nucleotide sequence. As shown in FIG. 3H, a ds-siNA may comprise (a) a sense strand consisting of 21 nucleotides, wherein 2′-fluoro nucleotides are at positions 7 and 9-11 from the 5′ end of the sense strand, and wherein 2′-O methyl nucleotides are at positions 1-6, 8, and 12-21 from the 5′ end of the sense strand; and (b) an antisense strand consisting of 23 nucleotides, wherein 2′-flouro nucleodies are at positions 2, 6, 14, and 16 from the 5′ end of the antisense strand, and wherein 2′-O methyl nucleotides are at positions 1, 3-5, 7-13, 15, and 17-23 from the 5′ end of the antisense strand. Optionally, the nucleotides at positions 22 and 23 from the 5′ end of the antisense strand may be unlocked nucleotides. Optionally, the ds-siNA may further comprise a conjugated moiety attached to the 3′ end of the sense strand (not pictured). The ds-siNA may comprise a stabilizing end cap attached to the 5′ end of the antisense strand (pictured). The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 21 and 22, and positions 22 and 23 from the 5′ end of the antisense strand. In some embodiments, the 2′-O methyl nucleotide at position 1 from the 5′ end of the sense strand is further modified to contain a 5′ stabilizing end cap. In some embodiments, the 2′-O methyl nucleotide at position 1 from the 5′ end of the antisense strand is further modified to contain a 5′ stabilizing end cap. In some embodiments, the 2′-O methyl nucleotide at position 1 from the 5′ end of the sense strand is further modified to contain a phosphorylation blocker. In some embodiments, the 2′-O methyl nucleotide at position 1 from the 3′ end of the sense strand is further modified to contain a phosphorylation blocker. In some embodiments, the 2′-O methyl nucleotide at position 1 from the 5′ end of the antisense strand is further modified to contain a phosphorylation blocker. In some embodiments, the 2′-O methyl nucleotide at position 1 from the 3′ end of the antisense strand is further modified to contain a phosphorylation blocker. In some embodiments, the 2′-O methyl nucleotide at position 1 from the 5′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide, a d2vd3U nucleotide, an omeco-d3U nucleotide, a 4hU nucleotide, a v-mun nucleotide, a c20-4 h nucleotide, an omeco-munb nucleotide, or a d2vmA nucleotide. In some embodiments, the 2′-O methyl nucleotide at position 1 from the 5′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the sense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, the 2′-O methyl nucleotide at position 1 from the 3′ end of the antisense strand is a d2vd3 nucleotide, a d2vd3U nucleotide, an omeco-d3 nucleotide, an omeco-d3U nucleotide, a 4 h nucleotide, a 4hU nucleotide, a v-mun nucleotide, a c20-4 h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, or f2P nucleotide. In some embodiments, at least 1, 2, 3, 4 or more 2′-fluoro nucleotides on the antisense strand is a fB, fN, f(4nh)Q, f4P, or f2P nucleotide. In some embodiments, at least 1, 2, 3, 4 or more 2′-O methyl nucleotide on the sense or antisense strand is a 2′-O methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

Any of the siNAs disclosed herein may comprise a sense strand and an antisense strand. The sense strand may comprise a first nucleotide sequence that is 15 to 30 nucleotides in length. The antisense strand may comprise a second nucleotide sequence that is 15 to 30 nucleotides in length.

In some embodiments, the double-stranded short interfering nucleic acid (ds-siNA) molecule comprises: (a) a sense strand wherein at least one modified nucleotide is a 2′-O methyl nucleotide and the nucleotide at position 3, 5, 7, 8, 9, 10, 11, 12, 14, 17, and/or 19 from the 5′ end of the first nucleotide sequence is a 2′-fluoro nucleotide. In some embodiments, the double-stranded short interfering nucleic acid (ds-siNA) molecule comprises: (a) a sense strand wherein at least one modified nucleotide is a 2′-O methyl nucleotide and the nucleotide at position 7 from the 5′ end of the first nucleotide sequence is a 2′-fluoro nucleotide. In some embodiments, the double-stranded short interfering nucleic acid (ds-siNA) molecule comprises: (a) a sense strand wherein at least one modified nucleotide is a 2′-O methyl nucleotide and the nucleotide at position 7, 9, 10, and/or 11 from the 5′ end of the first nucleotide sequence is a 2′-fluoro nucleotide.

In some embodiments, the double-stranded short interfering nucleic acid (ds-siNA) molecule comprises: (b) an antisense strand wherein at least one modified nucleotide is a 2′-O methyl nucleotide and the nucleotide at position 2, 5, 6, 8, 10, 14, 16, 17, and/or 18 from the 5′ end of the second nucleotide sequence is a 2′-fluoro nucleotide. In some embodiments, the double-stranded short interfering nucleic acid (ds-siNA) molecule comprises: (b) an antisense strand wherein at least one modified nucleotide is a 2′-O methyl nucleotide and the nucleotide at position 2 of the second nucleotide sequence is a 2′-fluoro nucleotide.

In any embodiment, the ds-siNA molecule comprises 1 or more phosphorothioate internucleoside linkage. In any embodiment, the ds-siNA molecule comprises 1 or more mesyl phosphoroamidate internucleoside linkage. In any embodiment, the ds-siNA molecule may further comprise a phosphorylation blocker, a galactosamine, 5′-stabilized end cap, conjugated moiety, destabilized nucleotide, modified nucleotide, thermally destabilized nucleotide, or a combination to two or more thereof. In some embodiments, the sense strand further comprises a phosphorylation blocker or a galactosamine. In some embodiments, the antisense strand further comprises a 5′-stabilized end cap. In some embodiments, the sense strand further comprises a phosphorylation blocker or a galactosamine and the antisense strand further comprises a 5′-stabilized end cap.

The present technology provides compositions comprising one or more of the siNA molecules described herein. The present technology also provides compositions comprising two or more of the siNA molecules described herein.

The present technology provides compositions comprising any of the siNA molecule described and a pharmaceutically acceptable carrier or diluent.

The present technology provides compositions comprising two or more of the siNA molecules described herein for use as a medicament.

The present technology provides compositions comprising any of the siNA molecule described and a pharmaceutically acceptable carrier or diluent for use as a medicament.

The present technology provides methods of treating a disease in a subject in need thereof, the method comprising administering to the subject any of the siNA molecules described herein.

The present technology provides uses of any of the siNA molecules described herein in the manufacture of a medicament for treating a disease.

Short Interfering Nucleic Acid (siNA) Molecules

As indicated above, the present disclosure provides siNA molecules comprising modified nucleotides. Any of the siNA molecules described herein may be double-stranded siNA (ds-siNA) molecules. The terms “siNA molecules” and “ds-siNA molecules” may be used interchangeably. In some embodiments, the ds-siNA molecules comprise a sense strand and an antisense strand. The siNA may comprise any of the first nucleotide, second nucleotide, sense strand, or antisense strand sequences disclosed herein. The siNA may comprise 5 to 100, 5 to 90, 10 to 100, 10 to 90, 10 to 80, 10 to 70, 10 to 60, 10 to 50, 10 to 30, 10 to 25, 15 to 100, 15 to 90, 15 to 80, 15 to 70, 15 to 60, 15 to 50, 15 to 30, or 15 to 25 nucleotides. The siNA may comprise at least 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. The siNA may comprise less than or equal to 50, 45, 40, 39, 38, 37, 36, 35, 34, 33, 32, 31, 30, 29, 28, 27, 26, 25, 24, 23, 22, 21, 20, or 19 nucleotides. The nucleotides may be modified nucleotides. The siNA may be single stranded. The siNA may be double stranded.

siNA Sense Strand

Any of the siNA molecules described herein may comprise a sense strand. The sense strand may comprise a first nucleotide sequence. The first nucleotide sequence may be 15 to 30, 15 to 25, 15 to 23, 17 to 23, 19 to 23, or 19 to 21 nucleotides in length. In some embodiments, the first nucleotide sequence is 15, 16, 17, 18, 19, 20, 21, 22, 23, 24, 25, 26, 27, 28, 29, or 30 nucleotides in length. In some embodiments, the first nucleotide sequence is at least 19 nucleotides in length. In some embodiments, the first nucleotide sequence is at least 21 nucleotides in length.

In some embodiments, the sense strand is the same length as the first nucleotide sequence. In some embodiments, the sense strand is longer than the first nucleotide sequence. In some embodiments, the sense strand may further comprise 1, 2, 3, 4, or 5 or more nucleotides than the first nucleotide sequence. In some embodiments, the sense strand may further comprise a deoxyribonucleic acid (DNA). In some embodiments, the DNA is thymine (T). In some embodiments, the sense strand may further comprise a TT sequence. In some embodiments, the sense strand may further comprise one or more modified nucleotides that are adjacent to the first nucleotide sequence. In some embodiments, the one or more modified nucleotides are independently selected from any of the modified nucleotides disclosed herein (e.g., 2′-fluoro nucleotide, 2′-O methyl nucleotide, 2′-fluoro nucleotide mimic, 2′-O methyl nucleotide mimic, or a nucleotide comprising a modified nucleobase).

In some embodiments, the first nucleotide sequence comprises 15, 16, 17, 18, 19, 20, 21, 22, 23, or more modified nucleotides independently selected from a 2′-O methyl nucleotide and a 2′-fluoro nucleotide. In some embodiments, the first nucleotide sequence comprises 16, 17, 18, 19, 20, 21, 22, 23, or more modified nucleotides independently selected from a 2′-O methyl nucleotide and a 2′-fluoro nucleotide. In some embodiments, 70%, 75%, 80%, 85%, 90%, 95% or 100% of the nucleotides in the first nucleotide sequence are modified nucleotides independently selected from a 2′-O methyl nucleotide and a 2′-fluoro nucleotide. In some embodiments, 100% of the nucleotides in the first nucleotide sequence are modified nucleotides independently selected from a 2′-O methyl nucleotide and a 2′-fluoro nucleotide. In some embodiments, the 2′-O methyl nucleotide is a 2′-O methyl nucleotide mimic. In some embodiments, the 2′-fluoro nucleotide is a 2′-fluoro nucleotide mimic.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the first nucleotide sequence are 2′-O methyl nucleotides. In some embodiments, between about 2 to 20 modified nucleotides of the first nucleotide sequence are 2′-O methyl nucleotides. In some embodiments, between about 5 to 25 modified nucleotides of the first nucleotide sequence are 2′-O methyl nucleotides. In some embodiments, between about 10 to 25 modified nucleotides of the first nucleotide sequence are 2′-O methyl nucleotides. In some embodiments, between about 12 to 25 modified nucleotides of the first nucleotide sequence are 2′-O methyl nucleotides. In some embodiments, at least about 10, 11, 12, 13, 14, 15, 16, 17, 18, 19, 20, 21, or 22 modified nucleotides of the first nucleotide sequence are 2′-O methyl nucleotides. In some embodiments, at least about 12 modified nucleotides of the first nucleotide sequence are 2′-O methyl nucleotides. In some embodiments, at least about 13 modified nucleotides of the first nucleotide sequence are 2′-O methyl nucleotides. In some embodiments, at least about 14 modified nucleotides of the first nucleotide sequence are 2′-O methyl nucleotides. In some embodiments, at least about 15 modified nucleotides of the first nucleotide sequence are 2′-O methyl nucleotides. In some embodiments, at least about 16 modified nucleotides of the first nucleotide sequence are 2′-O methyl nucleotides. In some embodiments, at least about 17 modified nucleotides of the first nucleotide sequence are 2′-O methyl nucleotides. In some embodiments, at least about 18 modified nucleotides of the first nucleotide sequence are 2′-O methyl nucleotides. In some embodiments, at least about 19 modified nucleotides of the first nucleotide sequence are 2′-O methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O methyl nucleotides. In some embodiments, less than or equal to 21 modified nucleotides of the first nucleotide sequence are 2′-O methyl nucleotides. In some embodiments, less than or equal to 20 modified nucleotides of the first nucleotide sequence are 2′-O methyl nucleotides. In some embodiments, less than or equal to 19 modified nucleotides of the first nucleotide sequence are 2′-O methyl nucleotides. In some embodiments, less than or equal to 18 modified nucleotides of the first nucleotide sequence are 2′-O methyl nucleotides. In some embodiments, less than or equal to 17 modified nucleotides of the first nucleotide sequence are 2′-O methyl nucleotides. In some embodiments, less than or equal to 16 modified nucleotides of the first nucleotide sequence are 2′-O methyl nucleotides. In some embodiments, less than or equal to 15 modified nucleotides of the first nucleotide sequence are 2′-O methyl nucleotides. In some embodiments, less than or equal to 14 modified nucleotides of the first nucleotide sequence are 2′-O methyl nucleotides. In some embodiments, less than or equal to 13 modified nucleotides of the first nucleotide sequence are 2′-O methyl nucleotides. In some embodiments, at least one modified nucleotide of the first nucleotide sequence is a 2′-O methyl pyrimidine. In some embodiments, at least 5, 6, 7, 8, 9, or 10 modified nucleotides of the first nucleotide sequence are 2′-O methyl pyrimidines. In some embodiments, at least one modified nucleotide of the first nucleotide sequence is a 2′-O methyl purine. In some embodiments, at least 5, 6, 7, 8, 9, or 10 modified nucleotides of the first nucleotide sequence are 2′-O methyl purines. In some embodiments, the 2′-O methyl nucleotide is a 2′-O methyl nucleotide mimic.

In some embodiments, between 2 to 15 modified nucleotides of the first nucleotide sequence are 2′-fluoro nucleotides. In some embodiments, between 2 to 10 modified nucleotides of the first nucleotide sequence are 2′-fluoro nucleotides. In some embodiments, between 2 to 6 modified nucleotides of the first nucleotide sequence are 2′-fluoro nucleotides. In some embodiments, 1 to 6, 1 to 5, 1 to 4, or 1 to 3 modified nucleotides of the first nucleotide sequence are 2′-fluoro nucleotides. In some embodiments, at least 1, 2, 3, 4, 5, or 6 modified nucleotides of the first nucleotide sequence are 2′-fluoro nucleotides. In some embodiments, at least 2, 3, 4, 5, or 6 modified nucleotides of the first nucleotide sequence are 2′-fluoro nucleotides. In some embodiments, at least 1 modified nucleotide of the first nucleotide sequence is a 2′-fluoro nucleotide. In some embodiments, at least 2 modified nucleotides of the first nucleotide sequence are 2′-fluoro nucleotides. In some embodiments, at least 3 modified nucleotides of the first nucleotide sequence are 2′-fluoro nucleotides. In some embodiments, at least 4 modified nucleotides of the first nucleotide sequence are 2′-fluoro nucleotides. In some embodiments, at least 5 modified nucleotides of the first nucleotide sequence are 2′-fluoro nucleotides. In some embodiments, at least 6 modified nucleotides of the first nucleotide sequence are 2′-fluoro nucleotides. In some embodiments, 10, 9, 8, 7, 6, 5, 4, 3 or fewer modified nucleotides of the first nucleotide sequence are 2′-fluoro nucleotides. In some embodiments, 10, 9, 8, 7, 6, 5, 4, 3, or 2 modified nucleotides of the first nucleotide sequence are 2′-fluoro nucleotides. In some embodiments, 10 or fewer modified nucleotides of the first nucleotide sequence are 2′-fluoro nucleotides. In some embodiments, 7 or fewer modified nucleotides of the first nucleotide sequence are 2′-fluoro nucleotides. In some embodiments, 6 or fewer modified nucleotides of the first nucleotide sequence are 2′-fluoro nucleotides. In some embodiments, 5 or fewer modified nucleotides of the first nucleotide sequence are 2′-fluoro nucleotides. In some embodiments, 4 or fewer modified nucleotides of the first nucleotide sequence are 2′-fluoro nucleotides. In some embodiments, 3 or fewer modified nucleotides of the first nucleotide sequence are 2′-fluoro nucleotides. In some embodiments, 2 or fewer modified nucleotides of the first nucleotide sequence are 2′-fluoro nucleotides. In some embodiments, at least one modified nucleotide of the first nucleotide sequence is a 2′-fluoro pyrimidine. In some embodiments, 1, 2, 3, 4, 5, or 6 modified nucleotides of the first nucleotide sequence are 2′-fluoro pyrimidines. In some embodiments, at least one modified nucleotide of the first nucleotide sequence is a 2′-fluoro purine. In some embodiments, 1, 2, 3, 4, 5, or 6 modified nucleotides of the first nucleotide sequence are 2′-fluoro purines. In some embodiments, the 2′-fluoro nucleotide is a 2′-fluoro nucleotide mimic.

In some embodiments, the nucleotide at position 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, at least four nucleotides at positions 3, 5, 7, 8, 9, 10, 11, 12, 14, 17, and/or 19 from the 5′ end of the first nucleotide sequence are 2′-fluoro nucleotides. In some embodiments, at least five nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 3, 5, 7, 8, 9, 10, 11, 12, 14, 17, and/or 19 from the 5′ end of the first nucleotide sequence is a 2′-fluoro nucleotide. In some embodiments, the nucleotide at positions 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 5 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 10 from the 5′ end of the first nucleotide sequence is a 2′-fluoro nucleotide. In some embodiments, the nucleotide at position 11 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 14 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the nucleotide at position 19 from the 5′ end of the first nucleotide sequence is a 2′-fluoro nucleotide. In some embodiments, at least 1, 2, 3, 4, 5, 6, or 7 nucleotides at position 3, 5, 7, 8, 9, 10, 11, 12, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 3, 7, 8, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 5, 7, 8, and/or 9 from the 5′ end of the first nucleotide sequence is a 2′-fluoro nucleotide. In some embodiments, the nucleotide at position 5, 9, 10, 11, 12, and/or 19 from the 5′ end of the first nucleotide sequence is a 2′-fluoro nucleotide. In some embodiments, the nucleotide at position 5, 9, 10, 11, 14, and/or 19 from the 5′ end of the first nucleotide sequence is a 2′-fluoro nucleotide. In some embodiments, the nucleotide at position 5, 9, 10, and/or 11 from the 5′ end of the first nucleotide sequence is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

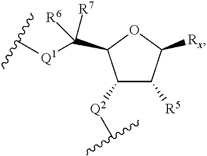

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

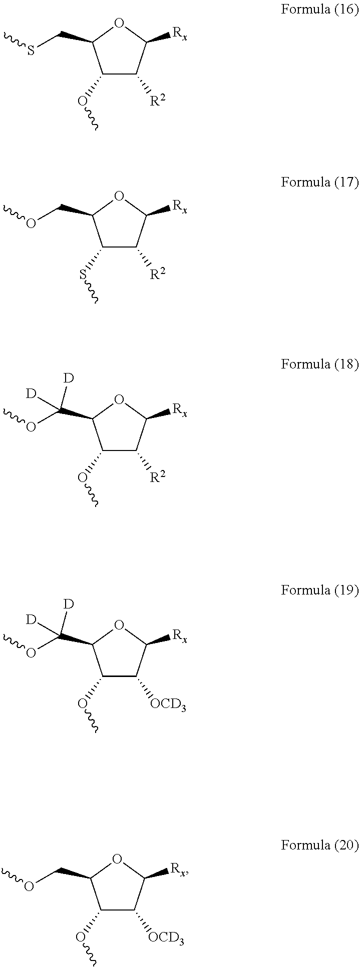

wherein Rₓ is independently a nucleobase and R² is F or —OCH₃. In some embodiments, the nucleobase is selected from thymine, cytosine, guanine, adenine, uracil, and an analogue or derivative thereof.

In some embodiments, the sense strand may comprise at least 1, at least 2, at least 3, at least 4, or at least 5 or more modified nucleotide(s) having the following chemical structure:

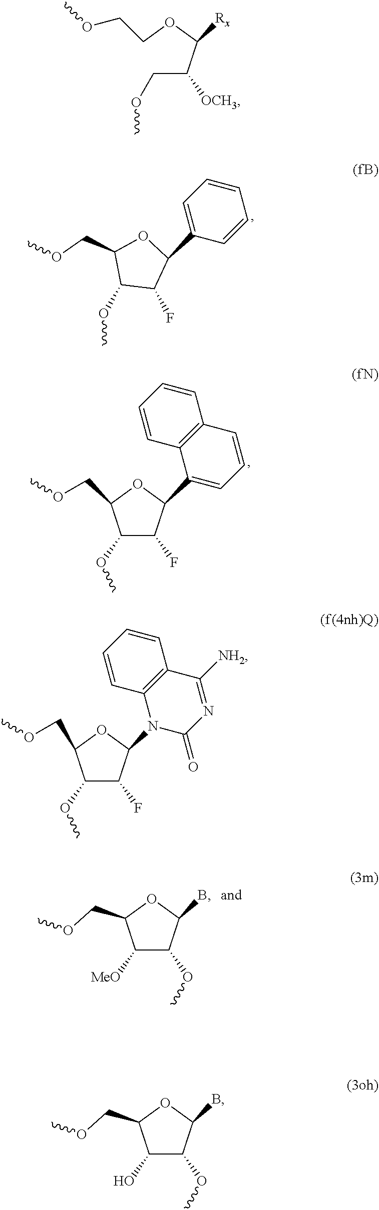

wherein B and Rₓ is a nucleobase, aryl, heteroaryl, or H. In some embodiments, the nucleobase is selected from thymine, cytosine, guanine, adenine, uracil, and an analogue or derivative thereof.

In some embodiments, the sense strand may comprise at least 1, at least 2, at least 3, at least 4, or at least 5 or more modified nucleotide(s) having the following chemical structure:

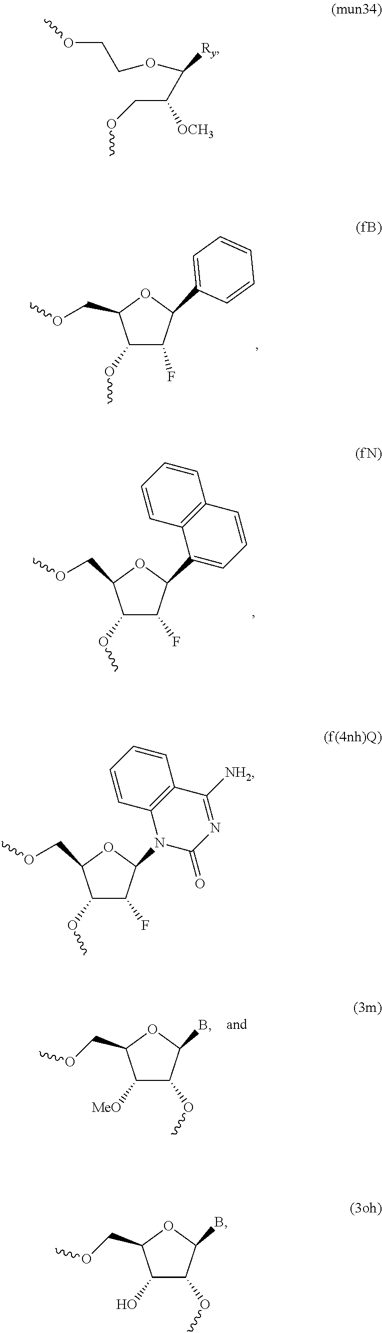

wherein B and R_{y} is a nucleobase. In some embodiments, the nucleobase is selected from thymine, cytosine, guanine, adenine, uracil, and an analogue or derivative thereof.

In some embodiments, the first nucleotide sequence comprises, consists of, or consists essentially of ribonucleic acids (RNAs). In some embodiments, the first nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the first nucleotide sequence are independently selected from 2′-O-methyl RNA and 2′-fluoro RNA.

In some embodiments, the sense strand may further comprise one or more internucleoside linkages independently selected from a phosphodiester (PO) internucleoside linkage, phosphorothioate (PS) internucleoside linkage, mesyl phosphoramidate internucleoside linkage (Ms), phosphorodithioate internucleoside linkage, and PS-mimic internucleoside linkage. In some embodiments, the PS-mimic internucleoside linkage is a sulfo internucleoside linkage.

In some embodiments, the sense strand may further comprise at least 1, 2, 3, 4, 5, 6,

7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of the first nucleotide sequence. In some embodiments, at least one phosphorothioate internucleoside linkage is between the nucleotides at positions 2 and 3 from the 5′ end of the first nucleotide sequence. In some embodiments, the sense strand comprises two phosphorothioate internucleoside linkages between the nucleotides at positions 1 to 3 from the 5′ end of the first nucleotide sequence.

In some embodiments, the sense strand may further comprise at least 1, 2, 3, 4, 5, 6, 7, 8, 9, 10, 11, 12, 13, 14, or 15 or more mesyl phosphoramidate internucleoside linkages. In some embodiments, the sense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the sense strand comprises 2 to 10, 2 to 8, 2 to 6, 1 to 5, 1 to 4, 1 to 3, or 1 to 2 mesyl phosphoramidate internucleoside linkages. In some embodiments, the sense strand comprises 1 to 2 mesyl phosphoramidate internucleoside linkages. In some embodiments, the sense strand comprises 2 to 4 mesyl phosphoramidate internucleoside linkages.

In some embodiments, the sense strand may comprise any of the modified nucleotides disclosed in the sub-section titled “Modified Nucleotides” below. In some embodiments, the sense stand may comprise a 5′-stabilized end cap, and the 5′-stabilized end cap may be selected from those disclosed in the sub-section titled “5′-Stabilized End Cap” below.

In some embodiments, any of the sense strands disclosed herein further comprise a TT sequence adjacent to the first nucleotide sequence.

siNA Antisense Strand

Any of the siNA molecules described herein may comprise an antisense strand. The antisense strand may comprise a second nucleotide sequence. The second nucleotide sequence may be 15 to 30, 15 to 25, 15 to 23, 17 to 23, 19 to 23, or 19 to 21 nucleotides in length. In some embodiments, the second nucleotide sequence is 15, 16, 17, 18, 19, 20, 21, 22, 23, 24, 25, 26, 27, 28, 29, or 30 nucleotides in length. In some embodiments, the second nucleotide sequence is at least 19 nucleotides in length. In some embodiments, the second nucleotide sequence is at least 21 nucleotides in length.

In some embodiments, the antisense strand is the same length as the second nucleotide sequence. In some embodiments, the antisense strand is longer than the second nucleotide sequence. In some embodiments, the antisense strand may further comprise 1, 2, 3, 4, or 5 or more nucleotides than the second nucleotide sequence. In some embodiments, the antisense strand is the same length as the sense strand. In some embodiments, the antisense strand is longer than the sense strand. In some embodiments, the antisense strand may further comprise 1, 2, 3, 4, or 5 or more nucleotides than the sense strand. In some embodiments, the antisense strand may further comprise a deoxyribonucleic acid (DNA). In some embodiments, the DNA is thymine (T). In some embodiments, the antisense strand may further comprise a TT sequence. In some embodiments, the antisense strand may further comprise one or more modified nucleotides that are adjacent to the second nucleotide sequence. In some embodiments, the one or more modified nucleotides are independently selected from any of the modified nucleotides disclosed herein (e.g., 2′-fluoro nucleotide, 2′-O methyl nucleotide, 2′-fluoro nucleotide mimic, 2′-O methyl nucleotide mimic, or a nucleotide comprising a modified nucleobase).

In some embodiments, the second nucleotide sequence comprises 15, 16, 17, 18, 19, 20, 21, 22, 23, or more modified nucleotides independently selected from a 2′-O methyl nucleotide and a 2′-fluoro nucleotide. In some embodiments, 70%, 75%, 80%, 85%, 90%, 95% or 100% of the nucleotides in the second nucleotide sequence are modified nucleotides independently selected from a 2′-O methyl nucleotide and a 2′-fluoro nucleotide. In some embodiments, 100% of the nucleotides in the second nucleotide sequence are modified nucleotides independently selected from a 2′-O methyl nucleotide and a 2′-fluoro nucleotide.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the second nucleotide sequence are 2′-O methyl nucleotides. In some embodiments, between about 2 to 20 modified nucleotides of the second nucleotide sequence are 2′-O methyl nucleotides. In some embodiments, between about 5 to 25 modified nucleotides of the second nucleotide sequence are 2′-O methyl nucleotides. In some embodiments, between about 10 to 25 modified nucleotides of the second nucleotide sequence are 2′-O methyl nucleotides. In some embodiments, between about 12 to 25 modified nucleotides of the second nucleotide sequence are 2′-O methyl nucleotides. In some embodiments, at least 10, 11, 12, 13, 14, 15, 16, 17, 18, 19, 20, 21, or 22 modified nucleotides of the second nucleotide sequence are 2′-O methyl nucleotides. In some embodiments, the second nucleotide sequence comprises 16, 17, 18, 19, 20, 21, 22, 23, or more modified nucleotides independently selected from a 2′-O methyl nucleotide and a 2′-fluoro nucleotide. In some embodiments, at least about 12 modified nucleotides of the second nucleotide sequence are 2′-O methyl nucleotides. In some embodiments, at least about 13 modified nucleotides of the second nucleotide sequence are 2′-O methyl nucleotides. In some embodiments, at least about 14 modified nucleotides of the second nucleotide sequence are 2′-O methyl nucleotides. In some embodiments, at least about 15 modified nucleotides of the second nucleotide sequence are 2′-O methyl nucleotides. In some embodiments, at least about 16 modified nucleotides of the second nucleotide sequence are 2′-O methyl nucleotides. In some embodiments, at least about 17 modified nucleotides of the second nucleotide sequence are 2′-O methyl nucleotides. In some embodiments, at least about 18 modified nucleotides of the second nucleotide sequence are 2′-O methyl nucleotides. In some embodiments, at least about 19 modified nucleotides of the second nucleotide sequence are 2′-O methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O methyl nucleotides. In some embodiments, less than or equal to 21 modified nucleotides of the second nucleotide sequence are 2′-O methyl nucleotides. In some embodiments, less than or equal to 20 modified nucleotides of the second nucleotide sequence are 2′-O methyl nucleotides. In some embodiments, less than or equal to 19 modified nucleotides of the second nucleotide sequence are 2′-O methyl nucleotides. In some embodiments, less than or equal to 18 modified nucleotides of the second nucleotide sequence are 2′-O methyl nucleotides. In some embodiments, less than or equal to 17 modified nucleotides of the second nucleotide sequence are 2′-O methyl nucleotides. In some embodiments, less than or equal to 16 modified nucleotides of the second nucleotide sequence are 2′-O methyl nucleotides. In some embodiments, less than or equal to 15 modified nucleotides of the second nucleotide sequence are 2′-O methyl nucleotides. In some embodiments, less than or equal to 14 modified nucleotides of the second nucleotide sequence are 2′-O methyl nucleotides. In some embodiments, less than or equal to 13 modified nucleotides of the second nucleotide sequence are 2′-O methyl nucleotides. In some embodiments, at least one modified nucleotide of the second nucleotide sequence is a 2′-O methyl pyrimidine. In some embodiments, at least 5, 6, 7, 8, 9, or 10 modified nucleotides of the second nucleotide sequence are 2′-O methyl pyrimidines. In some embodiments, at least one modified nucleotide of the second nucleotide sequence is a 2′-O methyl purine. In some embodiments, at least 5, 6, 7, 8, 9, or 10 modified nucleotides of the second nucleotide sequence are 2′-O methyl purines. In some embodiments, the 2′-O methyl nucleotide is a 2′-O methyl nucleotide mimic.

In some embodiments, between 2 to 15 modified nucleotides of the second nucleotide sequence are 2′-fluoro nucleotides. In some embodiments, between 2 to 10 modified nucleotides of the second nucleotide sequence are 2′-fluoro nucleotides. In some embodiments, between 2 to 6 modified nucleotides of the second nucleotide sequence are 2′-fluoro nucleotides. In some embodiments, 1 to 6, 1 to 5, 1 to 4, or 1 to 3 modified nucleotides of the second nucleotide sequence are 2′-fluoro nucleotides. In some embodiments, at least 1, 2, 3, 4, 5, or 6 modified nucleotides of the second nucleotide sequence are 2′-fluoro nucleotides. In some embodiments, at least 2, 3, 4, 5, or 6 modified nucleotides of the second nucleotide sequence are 2′-fluoro nucleotides. In some embodiments, at least 1 modified nucleotide of the second nucleotide sequence is a 2′-fluoro nucleotide. In some embodiments, at least 2 modified nucleotides of the second nucleotide sequence are 2′-fluoro nucleotides. In some embodiments, at least 3 modified nucleotides of the second nucleotide sequence are 2′-fluoro nucleotides. In some embodiments, at least 4 modified nucleotides of the second nucleotide sequence are 2′-fluoro nucleotides. In some embodiments, at least 5 modified nucleotides of the second nucleotide sequence are 2′-fluoro nucleotides. In some embodiments, 10, 9, 8, 7, 6, 5, 4, 3 or fewer modified nucleotides of the second nucleotide sequence are 2′-fluoro nucleotides. In some embodiments, less than or equal to 10, 9, 8, 7, 6, 5, 4, 3, or 2 modified nucleotides of the second nucleotide sequence are 2′-fluoro nucleotides. In some embodiments, 10 or fewer modified nucleotides of the second nucleotide sequence are 2′-fluoro nucleotides. In some embodiments, 7 or fewer modified nucleotides of the second nucleotide sequence are 2′-fluoro nucleotides. In some embodiments, 6 or fewer modified nucleotides of the second nucleotide sequence are 2′-fluoro nucleotides. In some embodiments, 5 or fewer modified nucleotides of the second nucleotide sequence are 2′-fluoro nucleotides. In some embodiments, 4 or fewer modified nucleotides of the second nucleotide sequence are 2′-fluoro nucleotides. In some embodiments, 3 or fewer modified nucleotides of the second nucleotide sequence are 2′-fluoro nucleotides. In some embodiments, 2 or fewer modified nucleotides of the second nucleotide sequence are 2′-fluoro nucleotides. In some embodiments, at least one modified nucleotide of the second nucleotide sequence is a 2′-fluoro pyrimidine. In some embodiments, 1, 2, 3, 4, 5, or 6 modified nucleotides of the second nucleotide sequence are 2′-fluoro pyrimidines. In some embodiments, at least one modified nucleotide of the second nucleotide sequence is a 2′-fluoro purine. In some embodiments, 1, 2, 3, 4, 5, or 6 modified nucleotides of the second nucleotide sequence are 2′-fluoro purines. In some embodiments, the 2′-fluoro nucleotide is a 2′-fluoro nucleotide mimic.

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

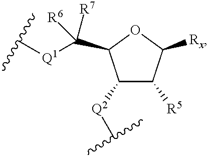

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16)-Formula (20):

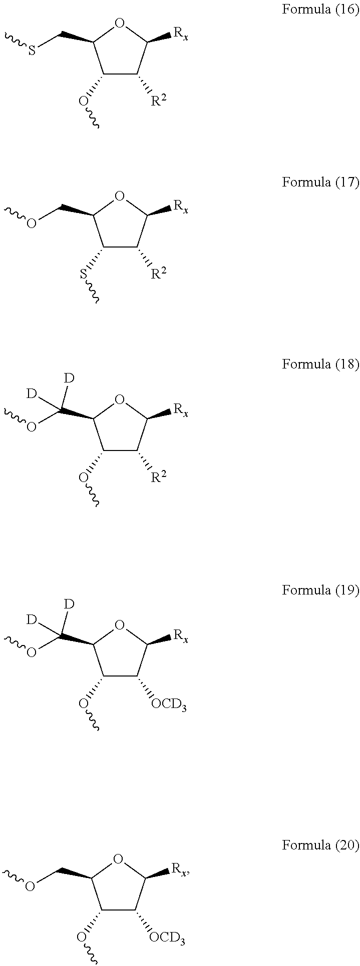

wherein Rₓ is a nucleobase and R² is independently F or —OCH₃. In some embodiments, the nucleobase is selected from thymine, cytosine, guanine, adenine, uracil, and an analogue or derivative thereof.

In some embodiments, the antisense strand may comprise at least 1, at least 2, at least 3, at least 4, or at least 5 or more modified nucleotide(s) having the following chemical structure:

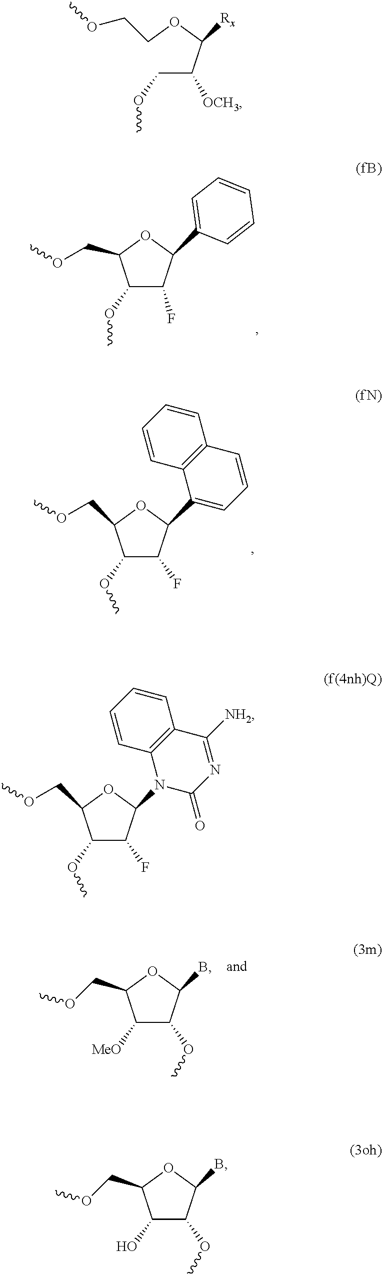

wherein B and Rx is a nucleobase, aryl, heteroaryl, or H. In some embodiments, the nucleobase is selected from thymine, cytosine, guanine, adenine, uracil, and an analogue or derivative thereof.

In some embodiments, the antisense strand may comprise at least 1, at least 2, at least 3, at least 4, or at least 5 or more modified nucleotide(s) having the following chemical structure:

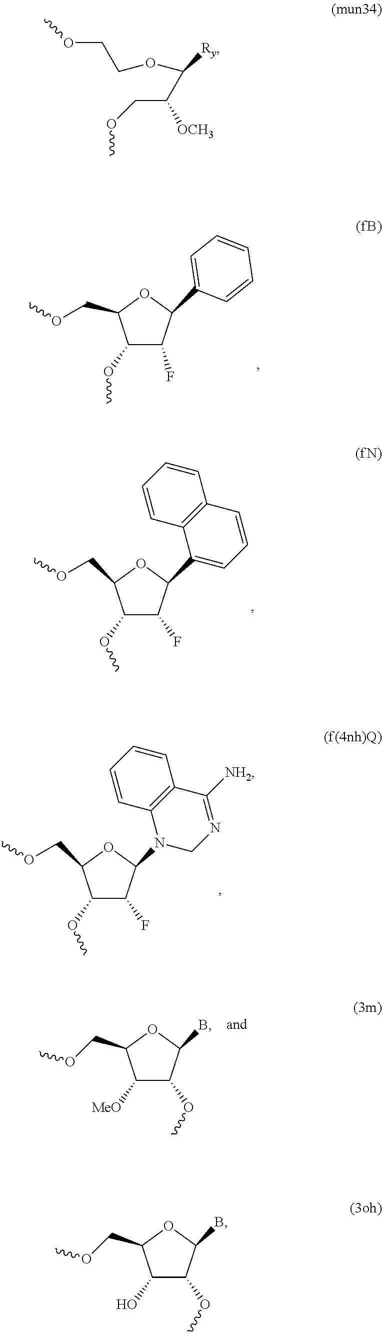

wherein B and R_{y} is a nucleobase. In some embodiments, the nucleobase is selected from thymine, cytosine, guanine, adenine, uracil, and an analogue or derivative thereof.

In some embodiments, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide. In some embodiments, the nucleotide at position 2, 5, 6, 8, 10, 14, 16, 17, and/or 18 from the 5′ end of the second nucleotide sequence is a 2′-fluoro nucleotide. In some embodiments, at least two nucleotides at positions 2, 5, 6, 8, 10, 14, 16, 17, and/or 18 from the 5′ end of the second nucleotide sequence are 2′-fluoro nucleotides. In some embodiments, at least three nucleotides at positions 2, 5, 6, 8, 10, 14, 16, 17, and/or 18 from the 5′ end of the second nucleotide sequence are 2′-fluoro nucleotides. In some embodiments, at least four nucleotides at positions 2, 5, 6, 8, 10, 14, 16, 17, and/or 18 from the 5′ end of the second nucleotide sequence are 2′-fluoro nucleotides. In some embodiments, at least five nucleotides at positions 2, 5, 6, 8, 10, 14, 16, 17, and/or 18 from the 5′ end of the second nucleotide sequence are 2′-fluoro nucleotides. In some embodiments, the nucleotides at positions 2 and/or 14 from the 5′ end of the second nucleotide sequence are 2′-fluoro nucleotides. In some embodiments, the nucleotides at positions 2, 6, and/or 16 from the 5′ end of the second nucleotide sequence are 2′-fluoro nucleotides. In some embodiments, the nucleotides at positions 2, 6, 14, and/or 16 from the 5′ end of the second nucleotide sequence are 2′-fluoro nucleotides. In some embodiments, the nucleotides at positions 2, 6, 10, 14, and/or 18 from the 5′ end of the second nucleotide sequence are 2′-fluoro nucleotides. In some embodiments, the nucleotides at positions 2, 5, 8, 14, and/or 17 from the 5′ end of the second nucleotide sequence are 2′-fluoro nucleotides. In some embodiments, the nucleotide at position 2 from the 5′ end of the second nucleotide sequence is a 2′-fluoro nucleotide. In some embodiments, the nucleotide at position 5 from the 5′ end of the second nucleotide sequence is a 2′-fluoro nucleotide. In some embodiments, the nucleotide at position 6 from the 5′ end of the second nucleotide sequence is a 2′-fluoro nucleotide. In some embodiments, the nucleotide at position 8 from the 5′ end of the second nucleotide sequence is a 2′-fluoro nucleotide. In some embodiments, the nucleotide at position 10 from the 5′ end of the second nucleotide sequence is a 2′-fluoro nucleotide. In some embodiments, the nucleotide at position 14 from the 5′ end of the second nucleotide sequence is a 2′-fluoro nucleotide. In some embodiments, the nucleotide at position 16 from the 5′ end of the second nucleotide sequence is a 2′-fluoro nucleotide. In some embodiments, the nucleotide at position 17 from the 5′ end of the second nucleotide sequence is a 2′-fluoro nucleotide. In some embodiments, the nucleotide at position 18 from the 5′ end of the second nucleotide sequence is a 2′-fluoro nucleotide. In some embodiments, the 2′-fluoro nucleotide is a 2′-fluoro nucleotide mimic.

In some embodiments, the nucleotides in the second nucleotide sequence are arranged in an alternating 1:3 modification pattern, wherein 1 nucleotide is a 2′-fluoro nucleotide and 3 nucleotides are 2′-O methyl nucleotides, and wherein the alternating 1:3 modification pattern occurs at least 2 times. In some embodiments, the alternating 1:3 modification pattern occurs 2-5 times. In some embodiments, at least two of the alternating 1:3 modification pattern occur consecutively. In some embodiments, at least two of the alternating 1:3 modification pattern occurs nonconsecutively. In some embodiments, at least 1, 2, 3, 4, or 5 alternating 1:3 modification pattern begins at nucleotide position 2, 6, 10, 14, and/or 18 from the 5′ end of the antisense strand. In some embodiments, at least one alternating 1:3 modification pattern begins at nucleotide position 2 from the 5′ end of the antisense strand. In some embodiments, wherein at least one alternating 1:3 modification pattern begins at nucleotide position 6 from the 5′ end of the antisense strand. In some embodiments, at least one alternating 1:3 modification pattern begins at nucleotide position 10 from the 5′ end of the antisense strand. In some embodiments, at least one alternating 1:3 modification pattern begins at nucleotide position 14 from the 5′ end of the antisense strand. In some embodiments, at least one alternating 1:3 modification pattern begins at nucleotide position 18 from the 5′ end of the antisense strand. In some embodiments, the 2′-fluoro nucleotide is a 2′-fluoro nucleotide mimic.

In some embodiments, the nucleotides in the second nucleotide sequence are arranged in an alternating 1:2 modification pattern, wherein 1 nucleotide is a 2′-fluoro nucleotide and 2 nucleotides are 2′-O methyl nucleotides, and wherein the alternating 1:2 modification pattern occurs at least 2 times. In some embodiments, the alternating 1:2 modification pattern occurs 2-5 times. In some embodiments, at least two of the alternating 1:2 modification pattern occurs consecutively. In some embodiments, at least two of the alternating 1:2 modification pattern occurs nonconsecutively. In some embodiments, at least 1, 2, 3, 4, or 5 alternating 1:2 modification pattern begins at nucleotide position 2, 5, 8, 14, and/or 17 from the 5′ end of the antisense strand. In some embodiments, at least one alternating 1:2 modification pattern begins at nucleotide position 2 from the 5′ end of the antisense strand. In some embodiments, at least one alternating 1:2 modification pattern begins at nucleotide position 5 from the 5′ end of the antisense strand. In some embodiments, at least one alternating 1:2 modification pattern begins at nucleotide position 8 from the 5′ end of the antisense strand. In some embodiments, at least one alternating 1:2 modification pattern begins at nucleotide position 14 from the 5′ end of the antisense strand. In some embodiments, at least one alternating 1:2 modification pattern begins at nucleotide position 17 from the 5′ end of the antisense strand. In some embodiments, the 2′-fluoro nucleotide is a 2′-fluoro nucleotide mimic.

In some embodiments, the second nucleotide sequence comprises, consists of, or consists essentially of ribonucleic acids (RNAs). In some embodiments, the second nucleotide sequence comprises, consists of, or consists essentially of modified RNAs. In some embodiments, the modified RNAs are selected from a 2′-O methyl RNA and 2′-fluoro RNA. In some embodiments, 15, 16, 17, 18, 19, 20, 21, 22, or 23 modified nucleotides of the second nucleotide sequence are independently selected from 2′-O methyl RNA and 2′-fluoro RNA. In some embodiments, the 2′-fluoro nucleotide is a 2′-fluoro nucleotide mimic.

In some embodiments, the sense strand may further comprise one or more internucleoside linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a sulfo internucleoside linkage.

In some embodiments, the antisense strand may further comprise at least 1, 2, 3, 4, 5, 6, 7, 8, 9, 10, 11, 12, 13, 14, or 15 or more phosphorothioate internucleoside linkages. In some embodiments, the antisense strand comprises 20, 19, 18, 17, 16, 15, 14, 13, 12, 11, 10, 9, 8, 7, 6, 5, 4, or 3 or fewer phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 8 phosphorothioate internucleoside linkages. In some embodiments, the antisense strand comprises 3 to 8 phosphorothioate internucleoside linkages. In some embodiments, the antisense strand comprises 4 to 8 phosphorothioate internucleoside linkages. In some embodiments, at least one phosphorothioate internucleoside linkage is between the nucleotides at positions 1 and 2 from the 5′ end of the second nucleotide sequence. In some embodiments, at least one phosphorothioate internucleoside linkage is between the nucleotides at positions 2 and 3 from the 5′ end of the second nucleotide sequence. In some embodiments, at least one phosphorothioate internucleoside linkage is between the nucleotides at positions 1 and 2 from the 3′ end of the second nucleotide sequence. In some embodiments, at least one phosphorothioate internucleoside linkage is between the nucleotides at positions 2 and 3 from the 3′ end of the second nucleotide sequence. In some embodiments, the antisense strand comprises two phosphorothioate internucleoside linkages between the nucleotides at positions 1 to 3 from the 5′ end of the first nucleotide sequence. In some embodiments, the antisense strand comprises two phosphorothioate internucleoside linkages between the nucleotides at positions 1 to 3 from the 3′ end of the first nucleotide sequence. In some embodiments, the antisense strand comprises (a) two phosphorothioate internucleoside linkages between the nucleotides at positions 1 to 3 from the 5′ end of the first nucleotide sequence; and (b) two phosphorothioate internucleoside linkages between the nucleotides at positions 1 to 3 from the 3′ end of the first nucleotide sequence.

In some embodiments, the antisense strand may further comprise at least 1, 2, 3, 4, 5, 6, 7, 8, 9, 10, 11, 12, 13, 14, or 15 or more mesyl phosphoramidate internucleoside linkages. In some embodiments, the antisense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 8 mesyl phosphoramidate internucleoside linkages. In some embodiments, the antisense strand comprises 3 to 8 mesyl phosphoramidate internucleoside linkages. In some embodiments, the antisense strand comprises 4 to 8 mesyl phosphoramidate internucleoside linkages.

In some embodiments, at least one end of the ds-siNA is a blunt end. In some embodiments, at least one end of the ds-siNA comprises an overhang, wherein the overhang comprises at least one nucleotide. In some embodiments, both ends of the ds-siNA comprise an overhang, wherein the overhang comprises at least one nucleotide. In some embodiments, the overhang comprises 1 to 5 nucleotides, 1 to 4 nucleotides, 1 to 3 nucleotides, or 1 to 2 nucleotides. In some embodiments, the overhang consists of 1 to 2 nucleotides.

In some embodiments, the sense strand may comprise any of the modified nucleotides disclosed in the sub-section titled “Modified Nucleotides” below. In some embodiments, the sense stand may comprise a 5′-stabilized end cap, and the 5′-stabilized end cap may be selected from those disclosed in the sub-section titled “5′-Stabilized End Cap” below.

In some embodiments, any of the antisense strands disclosed herein further comprise TT sequence adjacent to the second nucleotide sequence.

### Modified Nucleotides

The siNA molecules disclosed herein comprise one or more modified nucleotides. In some embodiments, the sense strands disclosed herein comprise one or more modified nucleotides. In some embodiments, any of the first nucleotide sequences disclosed herein comprise one or more modified nucleotides. In some embodiments, the antisense strands disclosed herein comprise one or more modified nucleotides. In some embodiments, any of the second nucleotide sequences disclosed herein comprise one or more modified nucleotides. In some embodiments, the one or more modified nucleotides is adjacent to the first nucleotide sequence. In some embodiments, at least one modified nucleotide is adjacent to the 5′ end of the first nucleotide sequence. In some embodiments, at least one modified nucleotide is adjacent to the 3′ end of the first nucleotide sequence. In some embodiments, at least one modified nucleotide is adjacent to the 5′ end of the first nucleotide sequence and at least one modified nucleotide is adjacent to the 3′ end of the first nucleotide sequence. In some embodiments, the one or more modified nucleotides is adjacent to the second nucleotide sequence. In some embodiments, at least one modified nucleotide is adjacent to the 5′ end of the second nucleotide sequence. In some embodiments, at least one modified nucleotide is adjacent to the 3′ end of the second nucleotide sequence. In some embodiments, at least one modified nucleotide is adjacent to the 5′ end of the second nucleotide sequence and at least one modified nucleotide is adjacent to the 3′ end of the second nucleotide sequence. In some embodiments, a 2′-O methyl nucleotide in any of sense strands or first nucleotide sequences disclosed herein is replaced with a modified nucleotide. In some embodiments, a 2′-O methyl nucleotide in any of antisense strands or second nucleotide sequences disclosed herein is replaced with a modified nucleotide.

In some embodiments, any of the siNA molecules, siNAs, sense strands, first nucleotide sequences, antisense strands, and second nucleotide sequences disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more modified nucleotides. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the siNA molecule, siNA, sense strand, first nucleotide sequence, antisense strand, or second nucleotide sequence are modified nucleotides.

In some embodiments, a modified nucleotide is selected from the group consisting of 2′-fluoro nucleotide, 2′-O methyl nucleotide, 2′-fluoro nucleotide mimic, 2′-O methyl nucleotide mimic, a locked nucleic acid, an unlocked nucleic acid, and a nucleotide comprising a modified nucleobase. In some embodiments, the unlocked nucleic acid is a 2′,3′-unlocked nucleic acid. In some embodiments, the unlocked nucleic acid is a 3′,4′-unlocked nucleic acid (e.g., mun34) in which the furanose ring lacks a bond between the 3′ and 4; carbons.

In some aspects, the siNA of the present disclosure will comprise at least one modified nucleotide selected from:

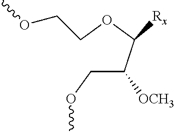

(wherein Rx is a nucleobase, aryl, heteroaryl, or H),

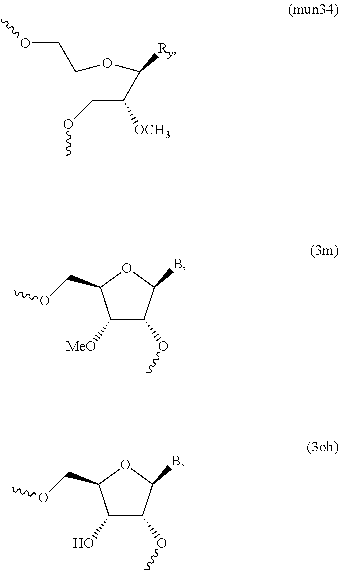

wherein B and R_{y} is a nucleobase, and

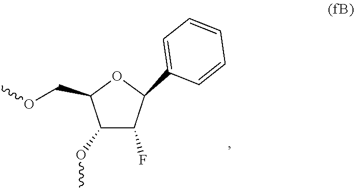

or combinations thereof. In some embodiments, the siNA may comprise at least 2, at least 3, at least 4, or at least 5 or more of these modified nucleotides. In some embodiments, the sense strand may comprise at least 1, at least 2, at least 3, at least 4, or at least 5 or more of

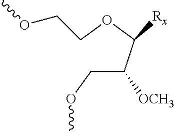

(wherein Rx is a nucleobase, aryl, heteroaryl, or H),

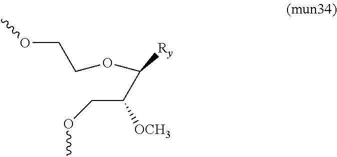

wherein B and R_{y} is a nucleobase, and

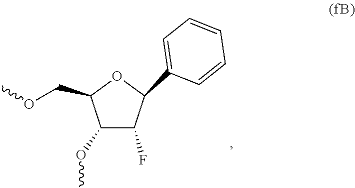

or combinations thereof. In some embodiments, the antisense strand may comprise at least 1, at least 2, at least 3, at least 4, or at least 5 or more of

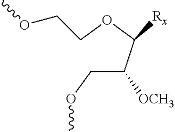

(wherein Rx is a nucleobase, aryl, heteroaryl, or H),

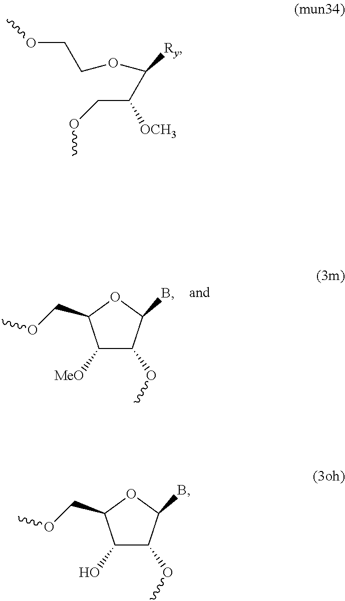

wherein B and R_{y} is a nucleobase, and

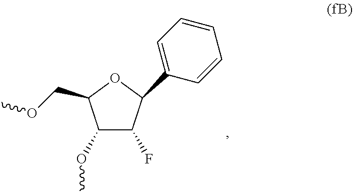

or combinations thereof. In some embodiments, both the sense strand and the antisense strand may each independently comprise at least 1, at least 2, at least 3, at least 4, or at least 5 or more of

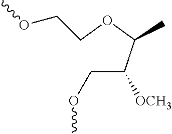

(wherein Rₓ is a nucleobase, aryl, heteroaryl, or H),

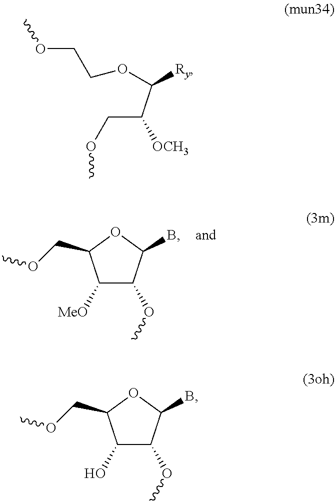

wherein B and R_{y} is a nucleobase, and

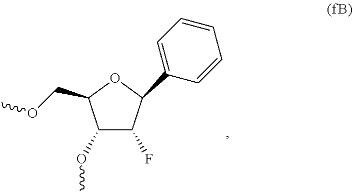

or combinations thereof. In some embodiments, the nucleobase is selected from thymine, cytosine, guanine, adenine, uracil, and an analogue or derivative thereof.

In some embodiments, any of the siNAs disclosed herein may additionally comprise other modified nucleotides, such as 2′-fluoro or 2′-O-methyl nucleotide mimics. For example, the disclosed siNA may comprise at least 1, 2, 3, 4, 5, 6, 7, 8, 9, or 10 or more 2′-fluoro or 2′-O-methyl nucleotide mimics. In some embodiments, any of the sense strands disclosed herein comprise at least 1, 2, 3, 4, 5, 6, 7, 8, 9, or 10 or more 2′-fluoro or 2′-O-methyl nucleotide mimics. In some embodiments, any of the first nucleotide sequences disclosed herein comprise at least 1, 2, 3, 4, 5, 6, 7, 8, 9, or 10 or more 2′-fluoro or 2′-O-methyl nucleotide mimics. In some embodiments, any of the antisense strand disclosed herein comprise at least 1, 2, 3, 4, 5, 6, 7, 8, 9, or 10 or more 2′-fluoro or 2′-O-methyl nucleotide mimics. In some embodiments, any of the second nucleotide sequences disclosed herein comprise at least 1, 2, 3, 4, 5, 6, 7, 8, 9, or 10 or more 2′-fluoro or 2′-O-methyl nucleotide mimics. In some embodiments, the 2′-fluoro or 2′-O-methyl nucleotide mimic is a nucleotide mimic of Formula (16)-Formula (20):

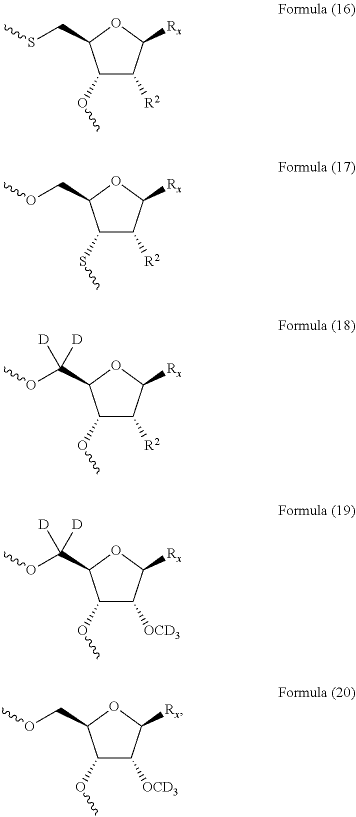

wherein Rₓ is a nucleobase and R² is independently F or —OCH₃. In some embodiments, the nucleobase is selected from thymine, cytosine, guanine, adenine, uracil, and an analogue or derivative thereof.

In some embodiments, the siNA molecules disclosed herein comprise at least one 2′-fluoro nucleotide, at least one 2′-O-methyl nucleotide, and at least one 2′-fluoro or 2′-O-methyl nucleotide mimic. In some embodiments, the at least one 2′-fluoro or 2′-O-methyl nucleotide mimic is adjacent to the first nucleotide sequence. In some embodiments, the at least one 2′-fluoro or 2′-O-methyl nucleotide mimic is adjacent to the 5′ end of first nucleotide sequence. In some embodiments, the at least one 2′-fluoro or 2′-O-methyl nucleotide mimic is adjacent to the 3′ end of first nucleotide sequence. In some embodiments, the at least one 2′-fluoro or 2′-O-methyl nucleotide mimic is adjacent to the second nucleotide sequence. In some embodiments, the at least one 2′-fluoro or 2′-O-methyl nucleotide mimic is adjacent to the 5′ end of second nucleotide sequence. In some embodiments, the at least one 2′-fluoro or 2′-O-methyl nucleotide mimic is adjacent to the 3′ end of second nucleotide sequence. In some embodiments, the first nucleotide sequence does not comprise a 2′-fluoro nucleotide mimic. In some embodiments, the first nucleotide sequence does not comprise a 2′-O-methyl nucleotide mimic. In some embodiments, the second nucleotide sequence does not comprise a 2′-fluoro nucleotide mimic. In some embodiments, the second nucleotide sequence does not comprise a 2′-O-methyl nucleotide mimic.

In some embodiments, any of the siNAs, sense strands, first nucleotide sequences, antisense strands, or second nucleotide sequences disclosed herein comprise at least one modified nucleotide that is

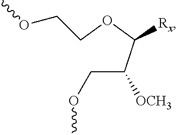

wherein Rx is a nucleobase, aryl, heteroaryl, or H;

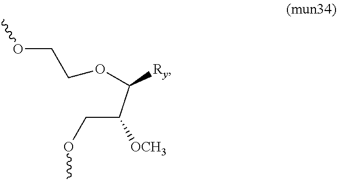

wherein R_{y} is a nucleobase, or

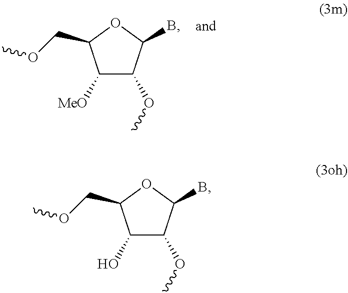

wherein B is a nucleobase.

### Phosphorylation Blocker

Further disclosed herein are siNA molecules comprising a phosphorylation blocker. In some embodiments, a 2′-O-methyl nucleotide in any of sense strands or first nucleotide sequences disclosed herein is replaced with a nucleotide containing a phosphorylation blocker. In some embodiments, a 2′-O-methyl nucleotide in any of antisense strands or second nucleotide sequences disclosed herein is replaced with a nucleotide containing a phosphorylation blocker. In some embodiments, a 2′-O-methyl nucleotide in any of sense strands or first nucleotide sequences disclosed herein is further modified to contain a phosphorylation blocker. In some embodiments, a 2′-O-methyl nucleotide in any of antisense strands or second nucleotide sequences disclosed herein is further modified to contain a phosphorylation blocker.

In some embodiments, any of the siNA molecules disclosed herein comprise a phosphorylation blocker of Formula (IV):

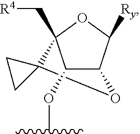

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring. In some embodiments, the nucleobase is selected from thymine, cytosine, guanine, adenine, uracil, and an analogue or derivative thereof.

In some embodiments, any of the siNA molecules disclosed herein comprise a phosphorylation blocker of Formula (IV):

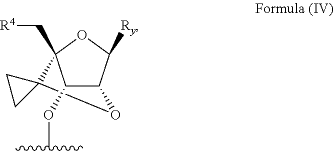

wherein R_{y} is a nucleobase, and R⁴ is —OCH₃ or —N(CH₂CH₂)₂O. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

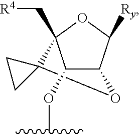

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring; and (b) a short interfering nucleic acid (siNA), wherein the phosphorylation blocker is conjugated to the siNA. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

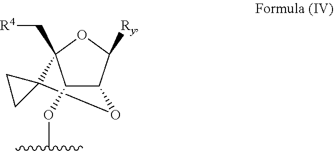

wherein R_{y} is a nucleobase, and R⁴ is —OCH₃ or —N(CH₂CH₂)₂O; and (b) a short interfering nucleic acid (siNA), wherein the phosphorylation blocker is conjugated to the siNA.

In some embodiments, the phosphorylation blocker is attached to the 3′ end of the sense strand or first nucleotide sequence. In some embodiments, the phosphorylation blocker is attached to the 3′ end of the sense strand or first nucleotide sequence via 1, 2, 3, 4, or 5 or more linkers. In some embodiments, the phosphorylation blocker is attached to the 5′ end of the sense strand or first nucleotide sequence. In some embodiments, the phosphorylation blocker is attached to the 5′ end of the sense strand or first nucleotide sequence via 1, 2, 3, 4, or 5 or more linkers. In some embodiments, the phosphorylation blocker is attached to the 3′ end of the antisense strand or second nucleotide sequence. In some embodiments, the phosphorylation blocker is attached to the 3′ end of the antisense strand or second nucleotide sequence via 1, 2, 3, 4, or 5 or more linkers. In some embodiments, the phosphorylation blocker is attached to the 5′ end of the antisense strand or second nucleotide sequence. In some embodiments, the phosphorylation blocker is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, mesyl phosphoramidate linker and phosphorodithioate linker.

### Conjugated Moiety

Further disclosed herein are siNA molecules comprising a conjugated moiety. In some embodiments, the conjugated moiety is selected from galactosamine, peptides, proteins, sterols, lipids, phospholipids, biotin, phenoxazines, active drug substance, cholesterols, phenanthridine, anthraquinone, acridine, fluoresceins, rhodamines, coumarins, and dyes. In some embodiments, the conjugated moiety is attached to the 3′ end of the sense strand or first nucleotide sequence. In some embodiments, the conjugated moiety is attached to the 3′ end of the sense strand or first nucleotide sequence via 1, 2, 3, 4, or 5 or more linkers. In some embodiments, the conjugated moiety is attached to the 5′ end of the sense strand or first nucleotide sequence. In some embodiments, the conjugated moiety is attached to the 5′ end of the sense strand or first nucleotide sequence via 1, 2, 3, 4, or 5 or more linkers. In some embodiments, the conjugated moiety is attached to the 3′ end of the antisense strand or second nucleotide sequence. In some embodiments, the conjugated moiety is attached to the 3′ end of the antisense strand or second nucleotide sequence via 1, 2, 3, 4, or 5 or more linkers. In some embodiments, the conjugated moiety is attached to the 5′ end of the antisense strand or second nucleotide sequence. In some embodiments, the conjugated moiety is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, phosphorodithioate linker, and mesyl phosphoramidate linker.

In some embodiments, the conjugated moiety is galactosamine. In some embodiments, any of the siNAs disclosed herein are attached to a conjugated moiety that is galactosamine. In some embodiments, the galactosamine is N-acetylgalactosamine (GalNAc). In some embodiments, any of the siNA molecules disclosed herein comprise GalNAc. In some embodiments, the GalNAc is of Formula (VI):

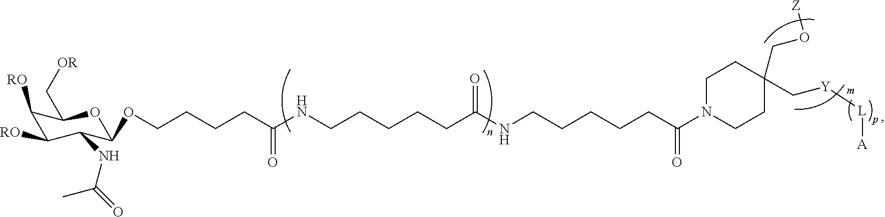

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. In some embodiments, the first protecting group is acetyl. In some embodiments, the second protecting group is trimethoxytrityl (TMT). In some embodiments, the activated group is a phosphoramidite group. In some embodiments, the phosphoramidite group is a cyanoethoxy N,N-diisopropylphosphoramidite group. In some embodiments, the linker is a C6-NH₂ group. In some embodiments, A is a short interfering nucleic acid (siNA) or siNA molecule. In some embodiments, m is 3. In some embodiments, R is H, Z is H, and n is 1. In some embodiments, R is H, Z is H, and n is 2.

In some embodiments, the GalNAc is of Formula (VII):

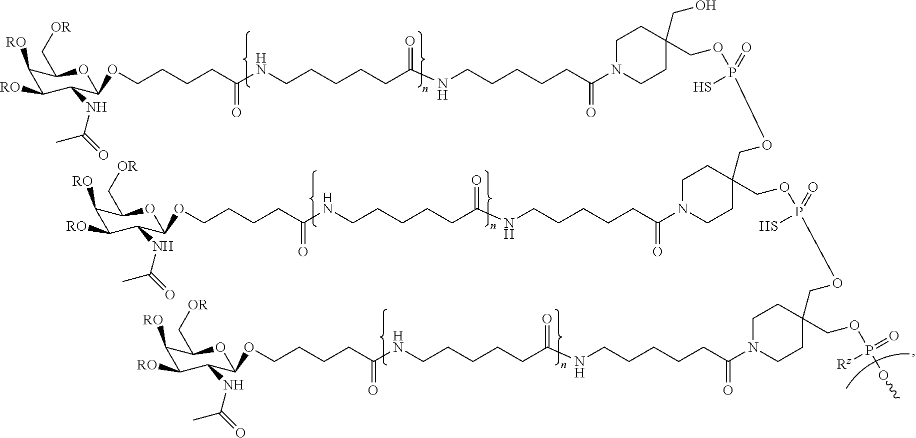

wherein R^{z} is OH or SH; and each n is independently 1 or 2.

In some embodiments, the galactosamine is attached to the 3′ end of the sense strand or first nucleotide sequence. In some embodiments, the galactosamine is attached to the 3′ end of the sense strand or first nucleotide sequence via 1, 2, 3, 4, or 5 or more linkers. In some embodiments, the galactosamine is attached to the 5′ end of the sense strand or first nucleotide sequence. In some embodiments, the galactosamine is attached to the 5′ end of the sense strand or first nucleotide sequence via 1, 2, 3, 4, or 5 or more linkers. In some embodiments, the galactosamine is attached to the 3′ end of the antisense strand or second nucleotide sequence. In some embodiments, the galactosamine is attached to the 3′ end of the antisense strand or second nucleotide sequence via 1, 2, 3, 4, or 5 or more linkers. In some embodiments, the galactosamine is attached to the 5′ end of the antisense strand or second nucleotide sequence. In some embodiments, the galactosamine is attached to the 5′ end of the antisense strand or second nucleotide sequence via 1, 2, 3, 4, or 5 or more linkers. In some embodiments, the one or more linkers are independently selected from the group consisting of a phosphodiester (p or po) linker, phosphorothioate (ps) linker, mesyl phosphoramidate linker (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

In some embodiments, the conjugated moiety is a lipid moiety. In some embodiments, any of the siNAs disclosed herein are attached to a conjugated moiety that is a lipid moiety. Examples of lipid moieties include, but are not limited to, a cholesterol moiety, a thioether, e.g., hexyl-S-tritylthiol, a thiocholesterol, an aliphatic chain, e.g., dodecandiol or undecyl residues a phospholipid, e.g., di-hexadecyl-rac-glycerol or triethylammonium 1-di-O-hexadecyl-rac-glycero-S—H-phosphonate, a polyamine or a polyethylene glycol chain, adamantane acetic acid, a palmityl moiety, or an octadecylamine or hexylamino-carbonyl-oxycholesterol moiety.

In some embodiments, the conjugated moiety is an active drug substance. In some embodiments, any of the siNAs disclosed herein are attached to a conjugated moiety that is an active drug substance. Examples of active drug substances include, but are not limited to, aspirin, warfarin, phenylbutazone, ibuprofen, suprofen, fenbufen, ketoprofen, (5)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

### 5′-Stabilized End Cap

Further disclosed herein are siNA molecules comprising a 5′-stabilized end cap. As used herein the terms “5′-stabilized end cap” and “5′ end cap” are used interchangeably. In some embodiments, a 2′-O methyl nucleotide in any of sense strands or first nucleotide sequences disclosed herein is replaced with a nucleotide containing a 5′-stabilized end cap. In some embodiments, a 2′-O methyl nucleotide in any of antisense strands or second nucleotide sequences disclosed herein is replaced with a nucleotide containing a 5′-stabilized end cap. In some embodiments, a 2′-O methyl nucleotide in any of sense strands or first nucleotide sequences disclosed herein is further modified to contain a 5′-stabilized end cap. In some embodiments, a 2′-O methyl nucleotide in any of antisense strands or second nucleotide sequences disclosed herein is further modified to contain a 5′-stabilized end cap.

In some embodiments, the 5′-stabilized end cap is a 5′ phosphate mimic. In some embodiments, the 5′-stabilized end cap is a modified 5′ phosphate mimic. In some embodiments, the modified 5′ phosphate is a chemically modified 5′ phosphate. In some embodiments, the 5′-stabilized end cap is a 5′-vinyl phosphonate. In some embodiments, the 5′-vinyl phosphonate is a 5′-(E)-vinyl phosphonate or 5′-(Z)-vinyl phosphonate. In some embodiments, the 5′-vinyl phosphonate is a deuterated vinyl phosphonate. In some embodiments, the deuterated vinyl phosphonate is a mono-deuterated vinyl phosphonate. In some embodiments, the deuterated vinyl phosphonate is a di-deuterated vinyl phosphonate. In some embodiments, the 5′-stabilized end cap is a phosphate mimic. Examples of phosphate mimics are disclosed in Parmar et al., 2018, J Med Chem, 61(3): 734-744, International Publication Nos. WO2018/045317 and WO2018/044350, and U.S. Pat. No. 10,087,210, each of which is incorporated by reference in its entirety.

In some aspects, the present disclosure provides siNA comprising a nucleotide phosphate mimic selected from:

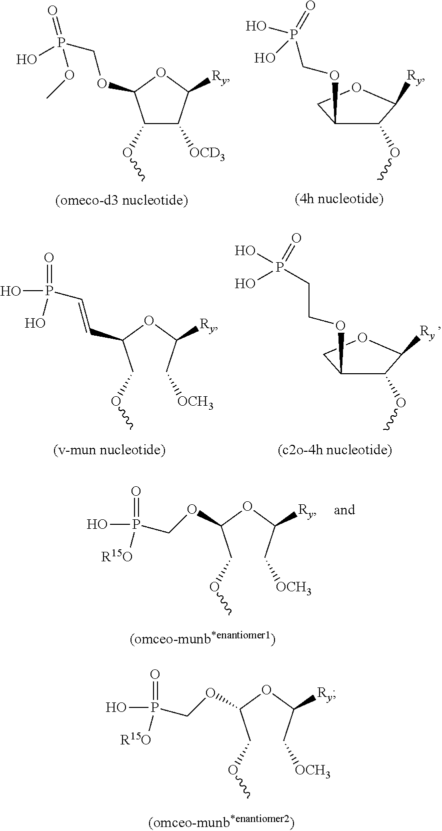

wherein R_{y} is a nucleobase and R¹⁵ is H or CH₃. In some embodiments, the nucleobase is selected from thymine, cytosine, guanine, adenine, uracil, and an analogue or derivative thereof. In some embodiments, the disclosed nucleotide phosphate mimics include, but are not limited to, the structures:

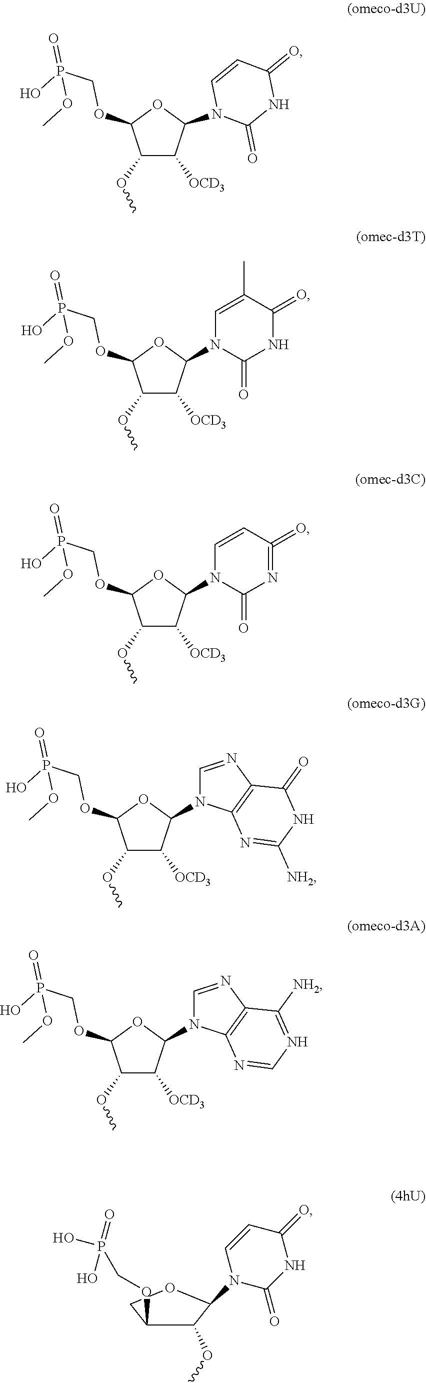

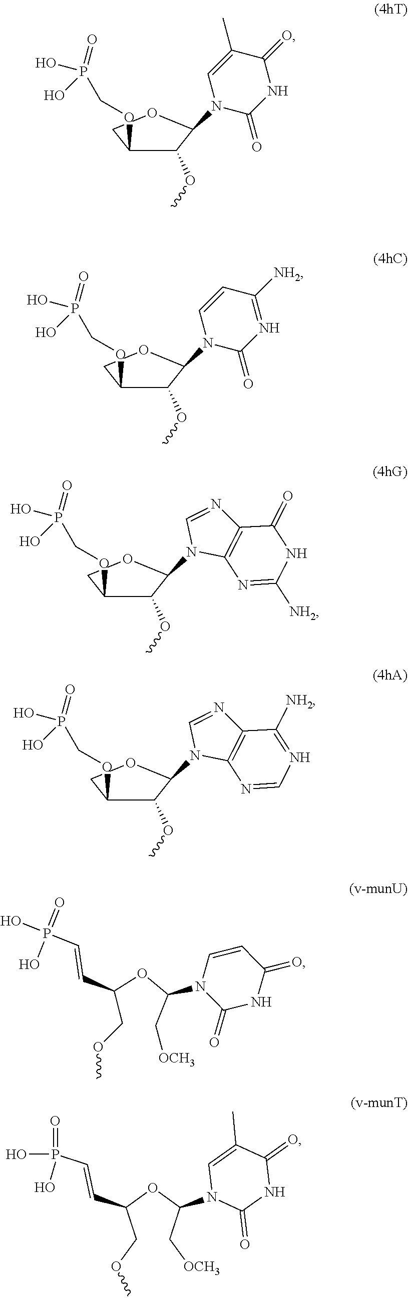

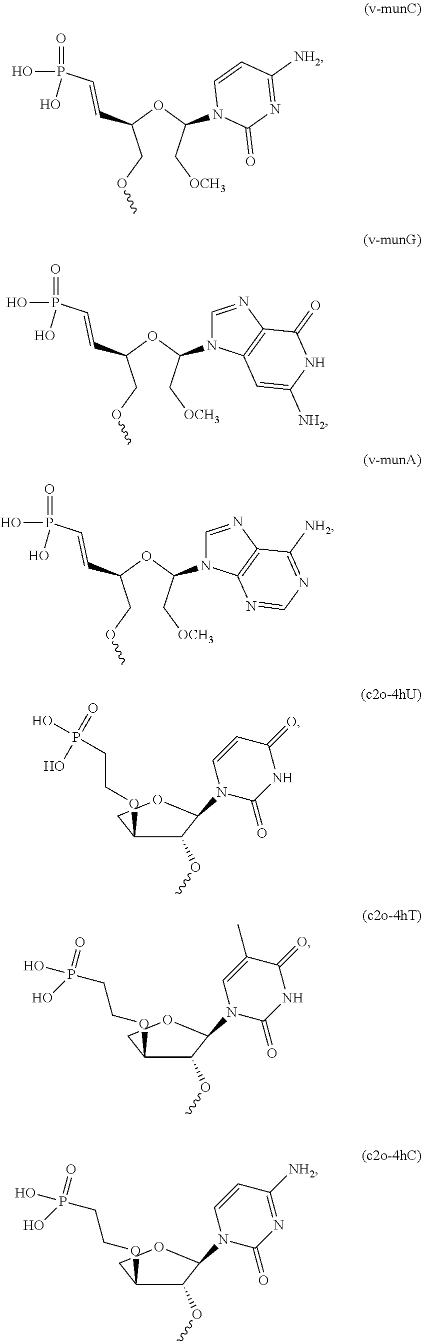

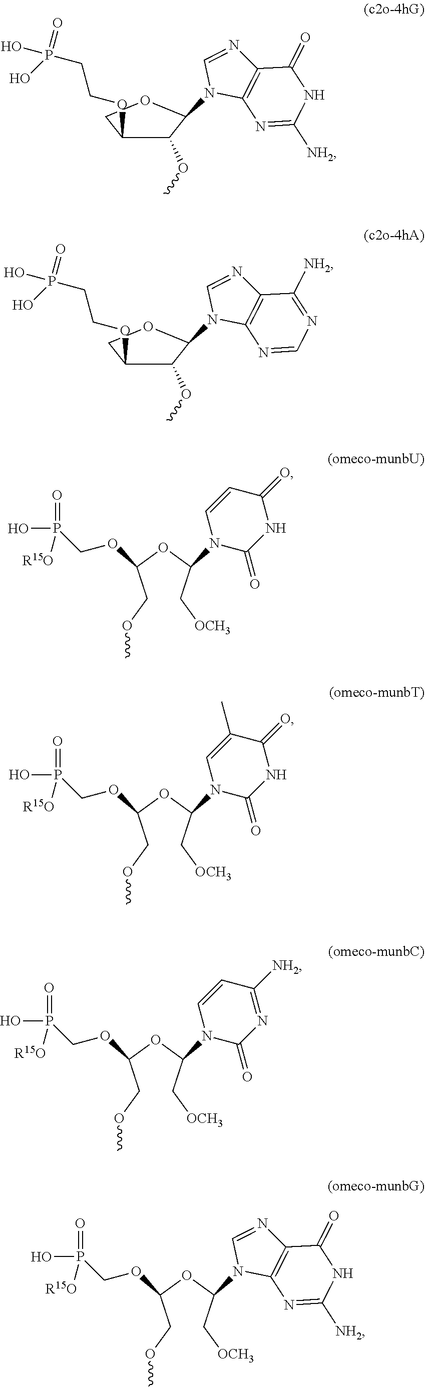

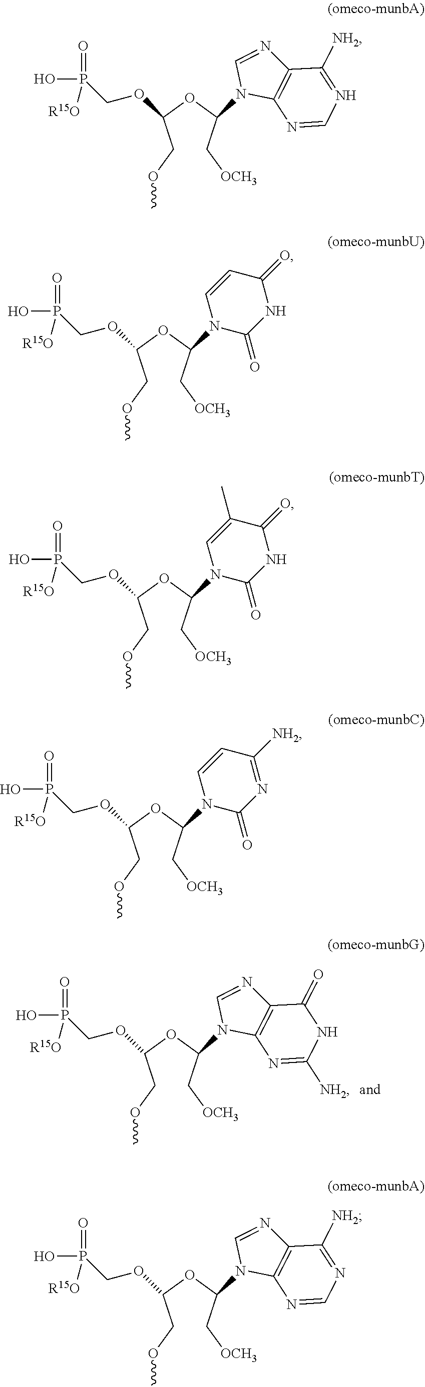

wherein R¹⁵ is H or CH₃.

In some aspects, the present disclosure provides siNA comprising a nucleotide phosphate mimic selected from:

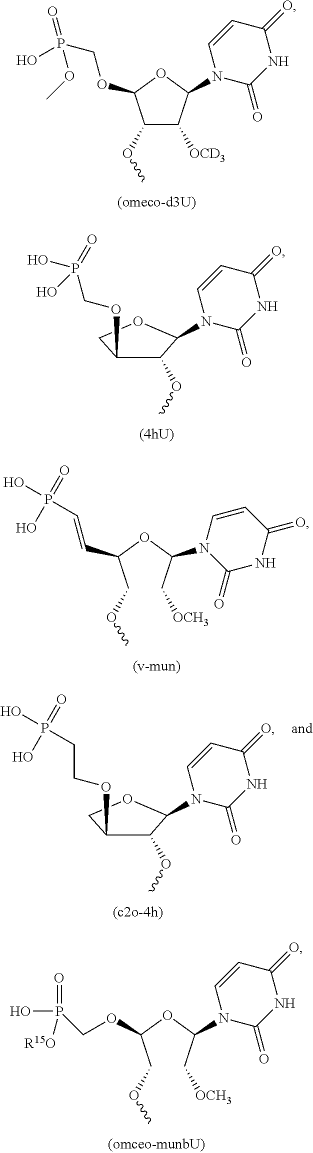

when R¹⁵ is CH₃); where R¹⁵ is H or CH₃. In some embodiments, one of these novel nucleotide phosphate mimics (e.g., omeco-d3 nucleotide, 4 h nucleotide, v-mun nucleotide, c20-4 h nucleotide, omeco-munb nucleotide, or d2vm nucleotide) are located at the 5′ end of the antisense strand; however, these novel nucleotide phosphate mimics may also be incorporated at the 5′ end of the sense strand, the 3′ end of the antisense strand, or the 3′ end of the sense strand.

Additionally or alternatively, the siNA molecules disclosed herein may comprise in the sense strand, the antisense strand, or both a 5′-stabilized end cap of Formula (Ia):

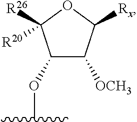

wherein Rₓ is H, a nucleobase, aryl, or heteroaryl; R²⁶ is

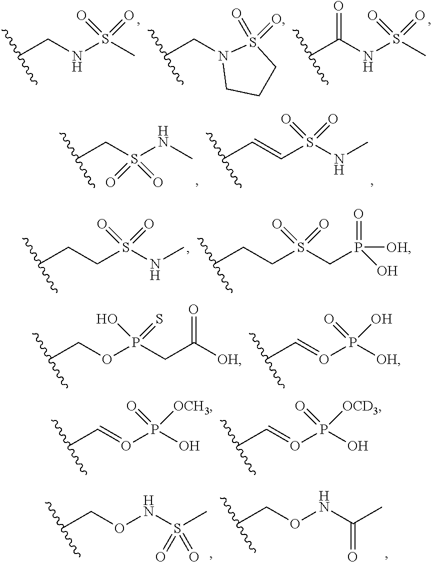

—CH═CD-Z, —CD═CH—Z, —CD═CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (Ib):

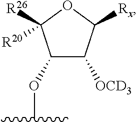

wherein Rₓ is H, a nucleobase, aryl, or heteroaryl; R²⁶ is

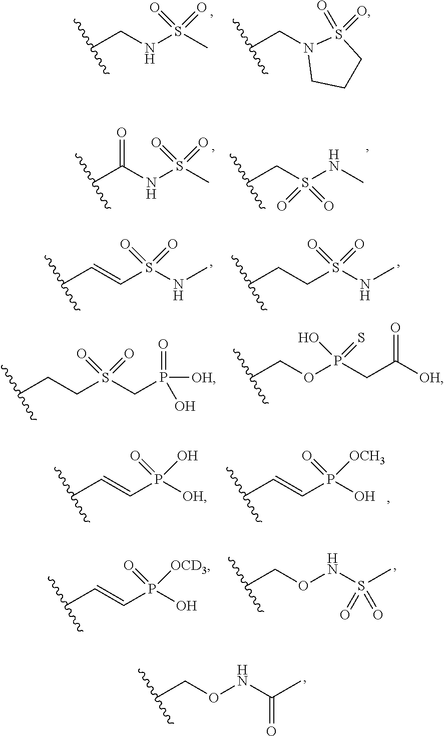

—CH═CD-Z, —CD═CH—Z, —CD═CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (Ic):

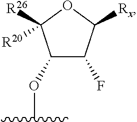

wherein Rₓ is a nucleobase, aryl, heteroaryl, or H, R²⁶ is

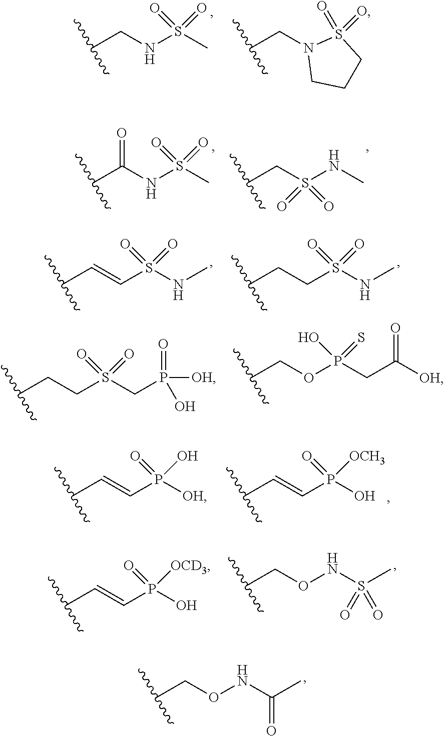

—CH═CD-Z, —CD═CH—Z, —CD═CD-Z, —(CR²¹R²²)ₙ—Z, or —(C₂-C₆ alkenylene)-Z and R²⁰ is hydrogen; or R²⁶ and R²⁰ together form a 3- to 7-membered carbocyclic ring substituted with —(CR²¹R²²), —Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, or —NR²³SO₂R²⁴; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or

R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIa):

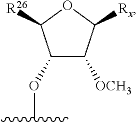

wherein Rₓ is a nucleobase, aryl, heteroaryl, or H, R²⁶ is

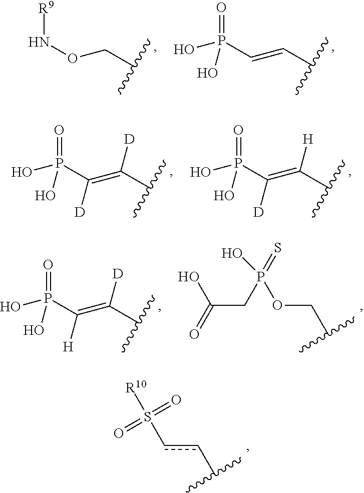

—CH₂SO₂NHCH₃, or

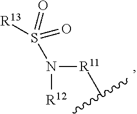

R⁹ is —SO₂CH₃ or —COCH₃, [symbol] is a double or single bond, R¹⁰═—CH₂PO₃H or —NHCH₃, R¹¹ is —CH₂— or —CO—, and R¹² is H and R¹³ is CH₃ or R¹² and R₁₃ together form —CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIb):

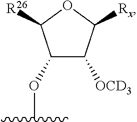

wherein Rₓ is a nucleobase, aryl, heteroaryl, or H, R²⁶ is

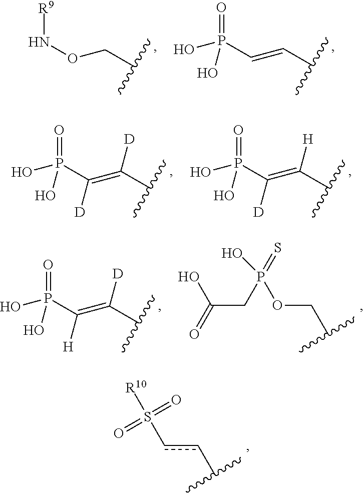

—CH₂SO₂NHCH₃, or

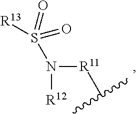

R⁹ is —SO₂CH₃ or —COCH₃, [symbol] is a double or single bond, R¹⁰═—CH₂PO₃H or —NHCH₃, R¹¹ is —CH₂— or —CO—, and R¹² is H and R¹³ is CH₃ or R¹² and R₁₃ together form —CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (III):

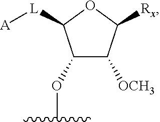

wherein Rₓ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂). In some embodiments, R¹ is an aryl. In some embodiments, the aryl is a phenyl.

Additionally or alternatively, the siNA molecules disclosed herein may comprise a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61) and Formula (62):

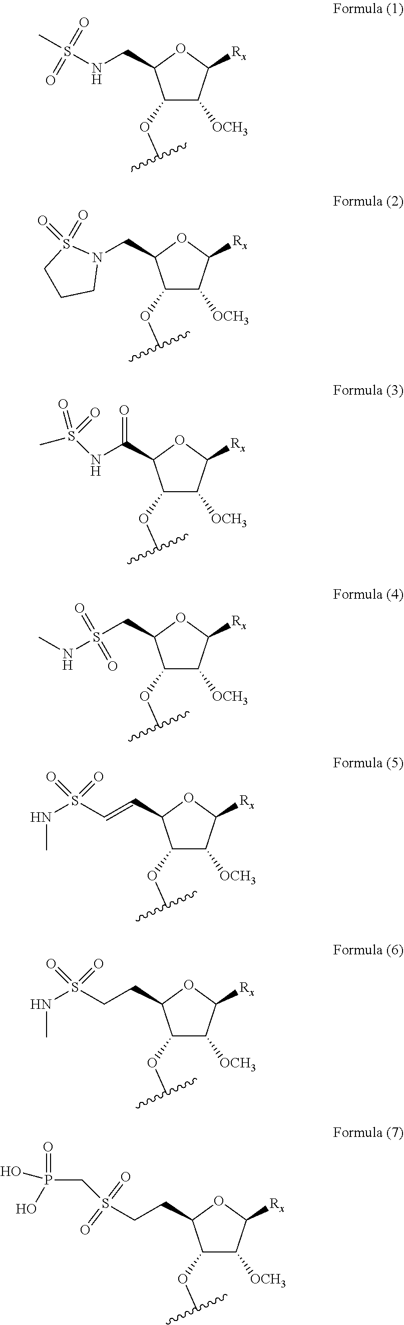

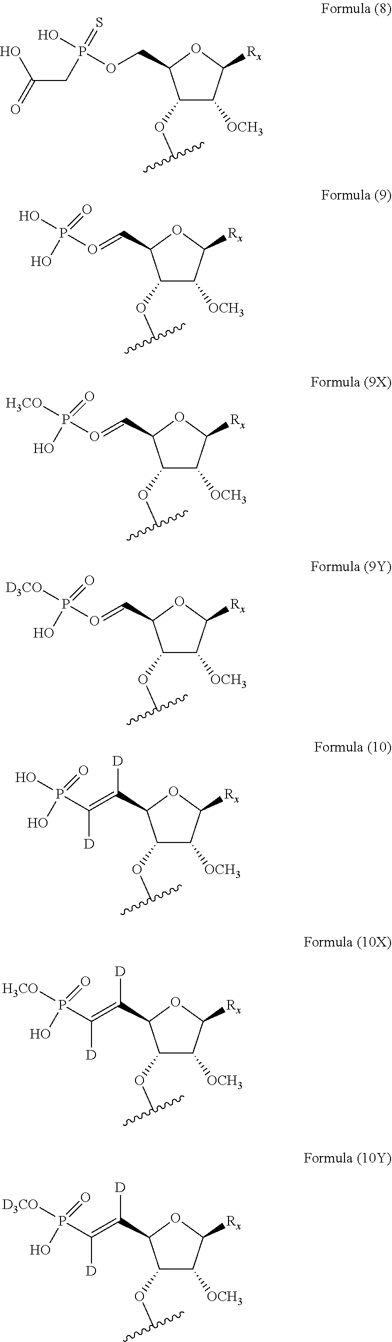

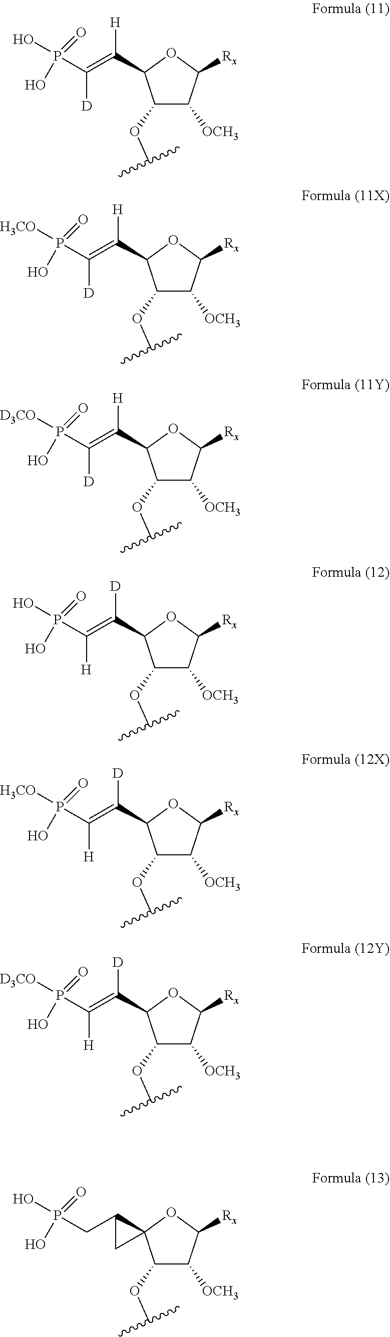

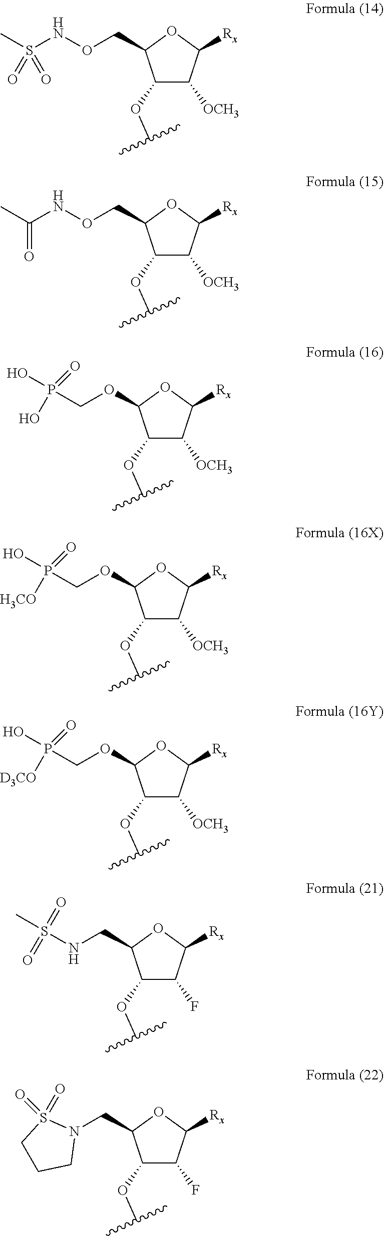

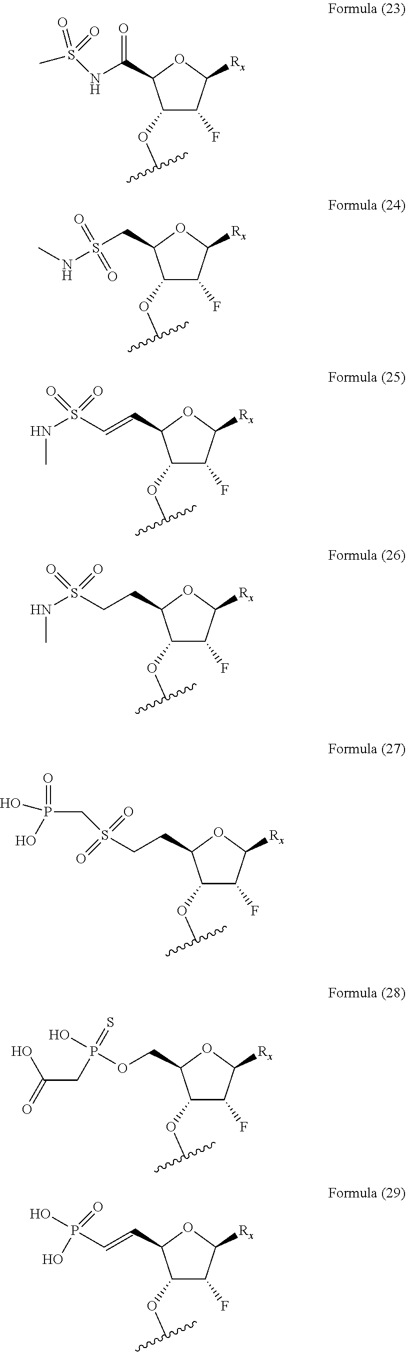

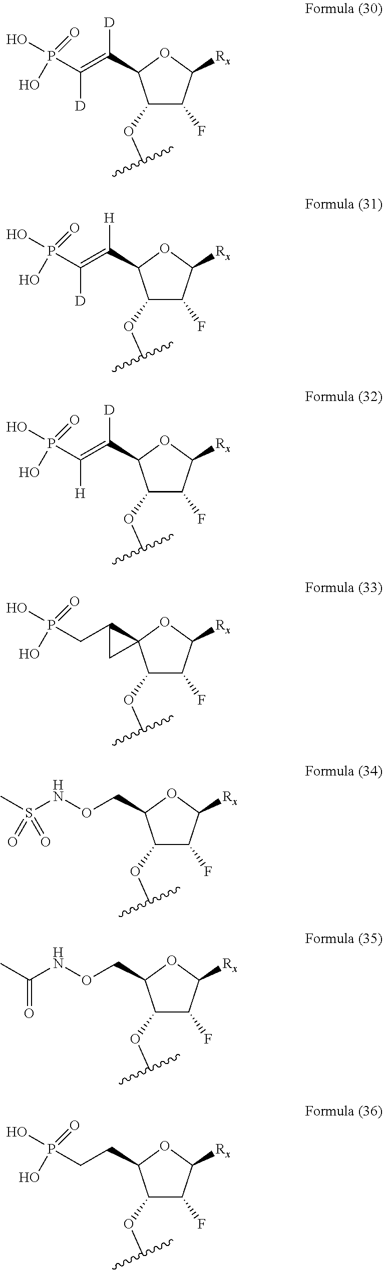

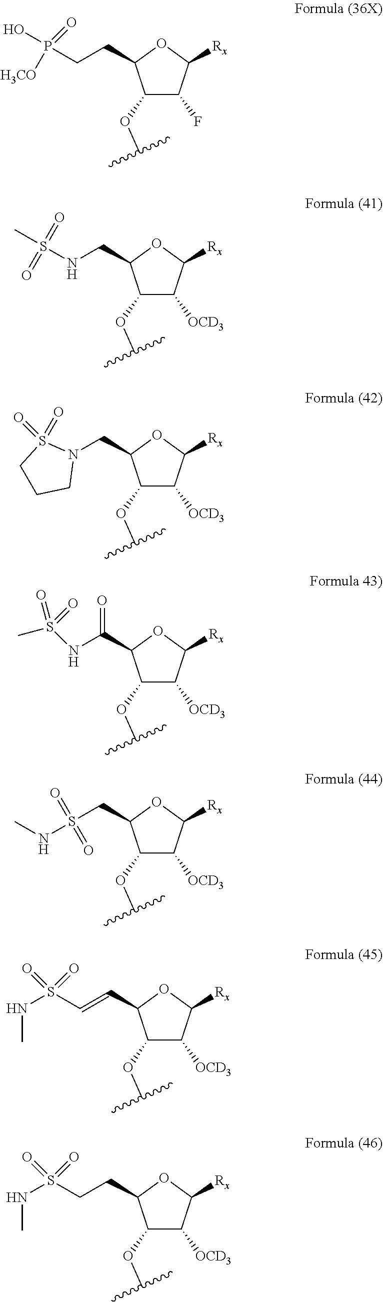

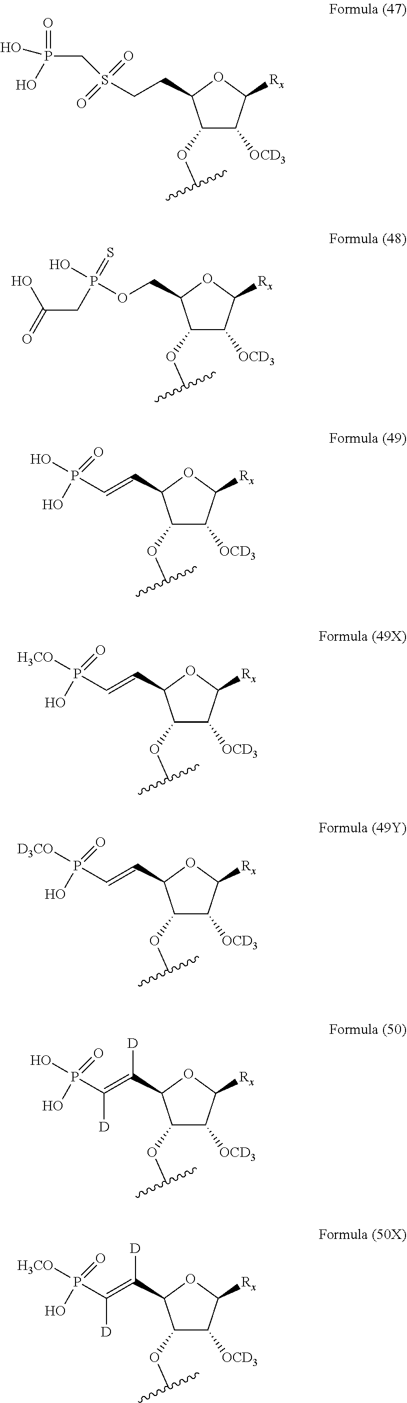

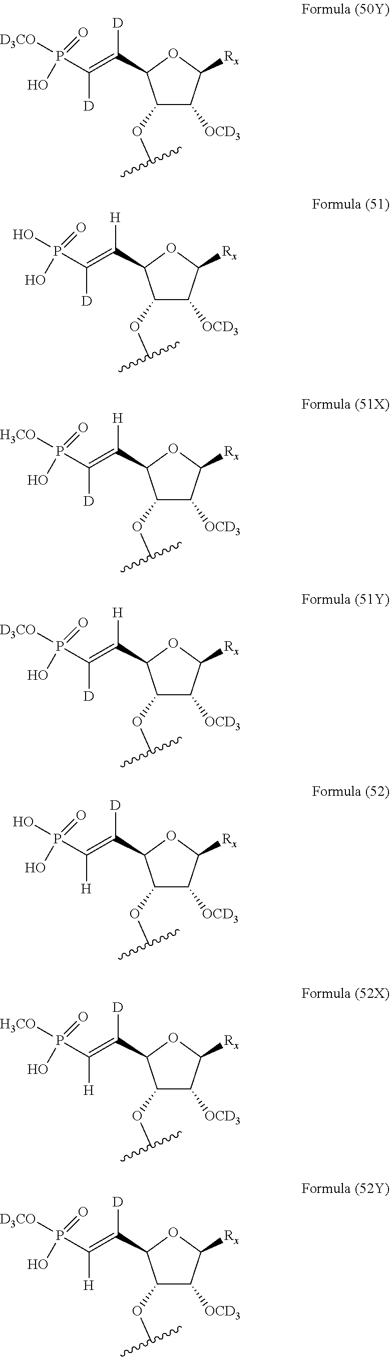

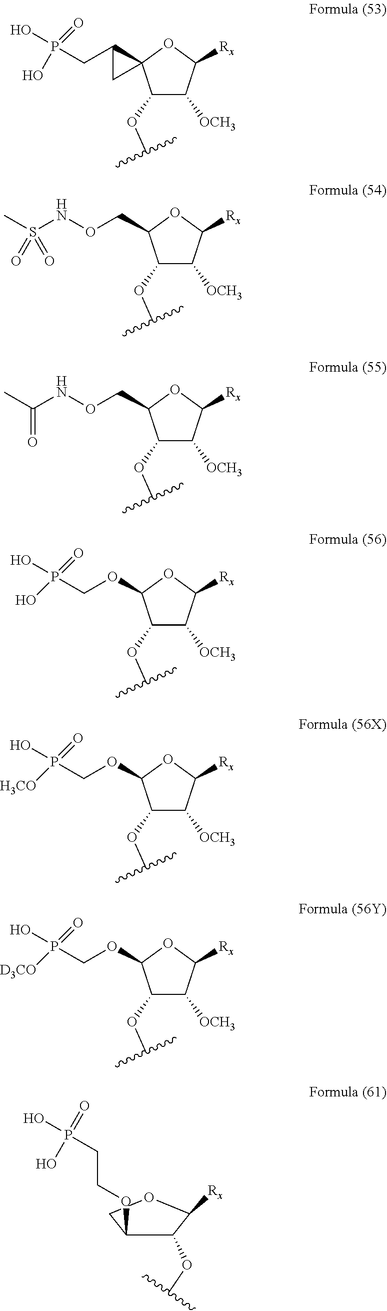

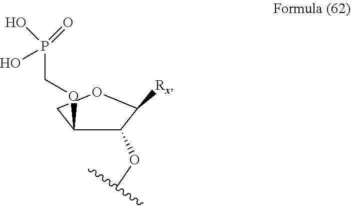

wherein Rₓ is a nucleobase, aryl, heteroaryl, or H.

In some embodiments, any of the siNA molecules disclosed herein comprise a 5′-stabilized end cap selected from the group consisting of Formula (50), Formula (50X), Formula (50Y), Formula (56), Formula (56X), Formula (56Y), Formula (61), and Formula (62):

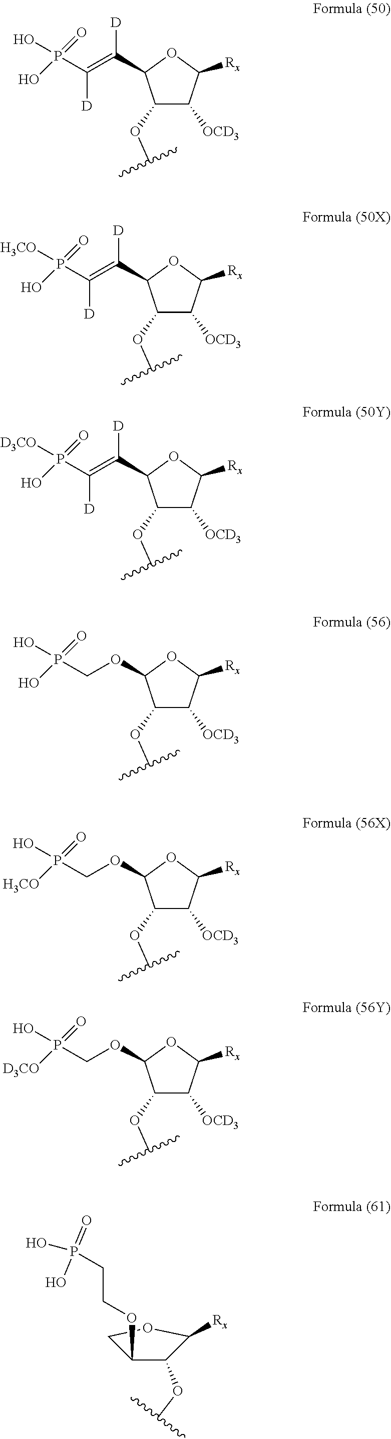

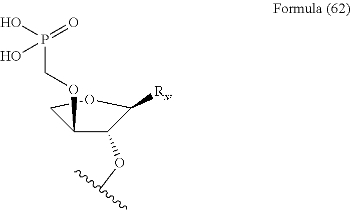

wherein Rₓ is a nucleobase, aryl, heteroaryl, or H.

In some embodiments, any of the siNA molecules disclosed herein comprise a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′:

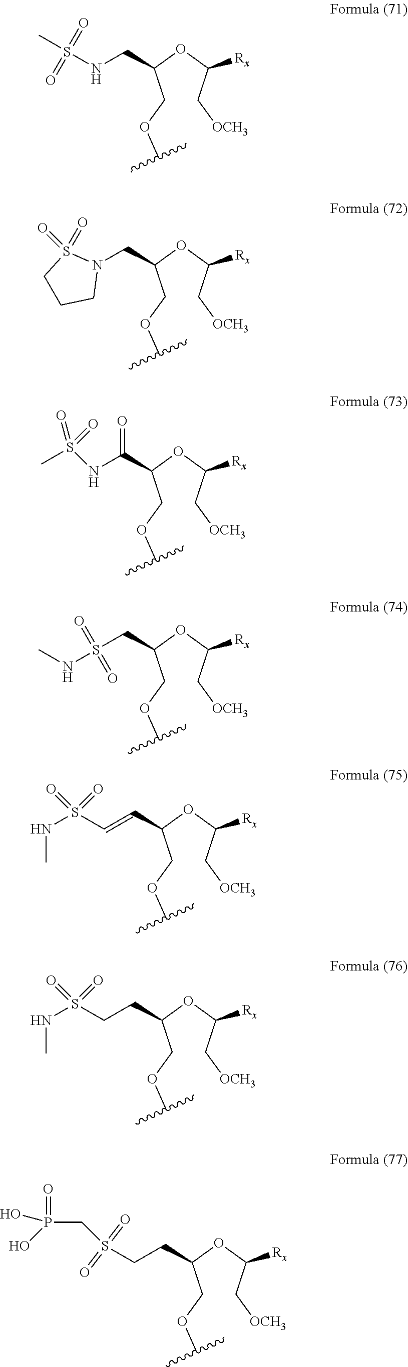

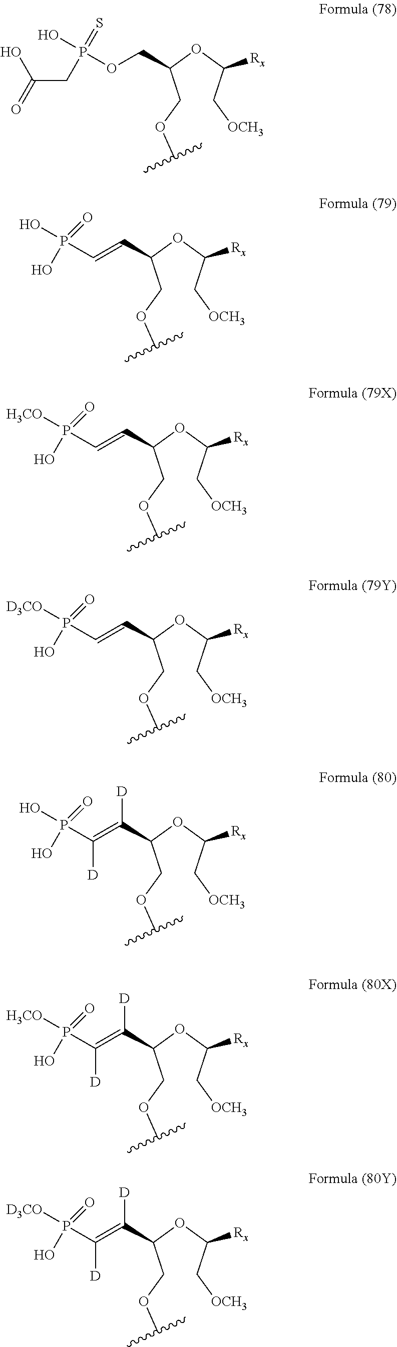

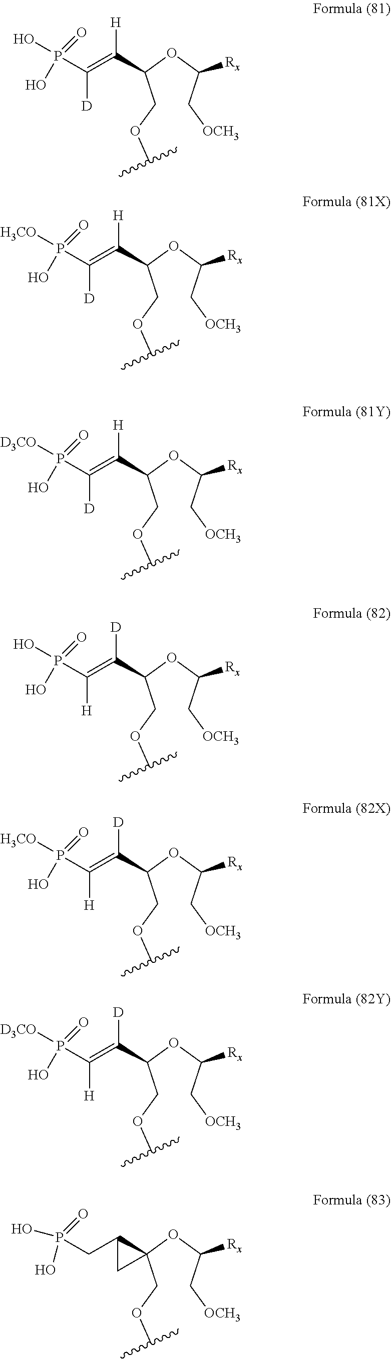

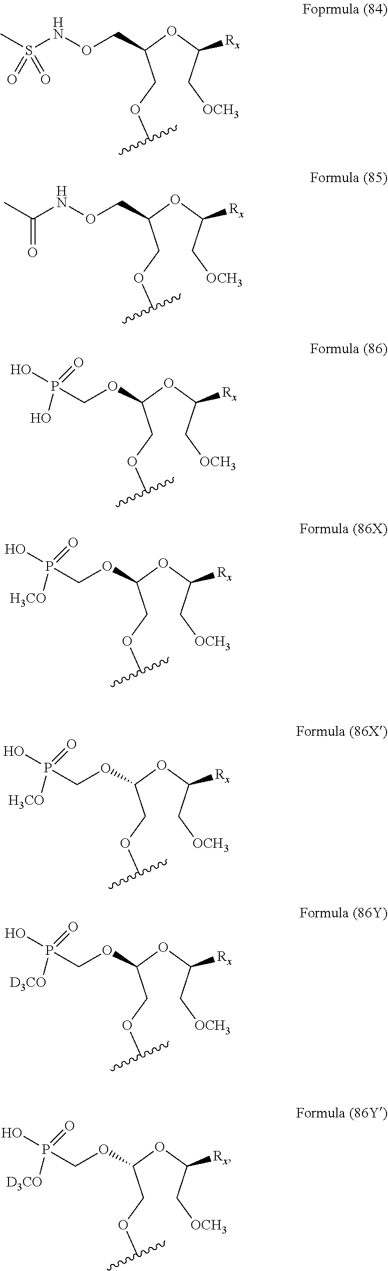

wherein Rₓ is a nucleobase, aryl, heteroaryl, or H.

In some embodiments, any of the siNA molecules disclosed herein comprise a 5′-stabilized end cap selected from the group consisting of Formula (78), Formula (79), Formula (79X), Formula (79Y), Formula (86), Formula (86X), and Formula (86X′):

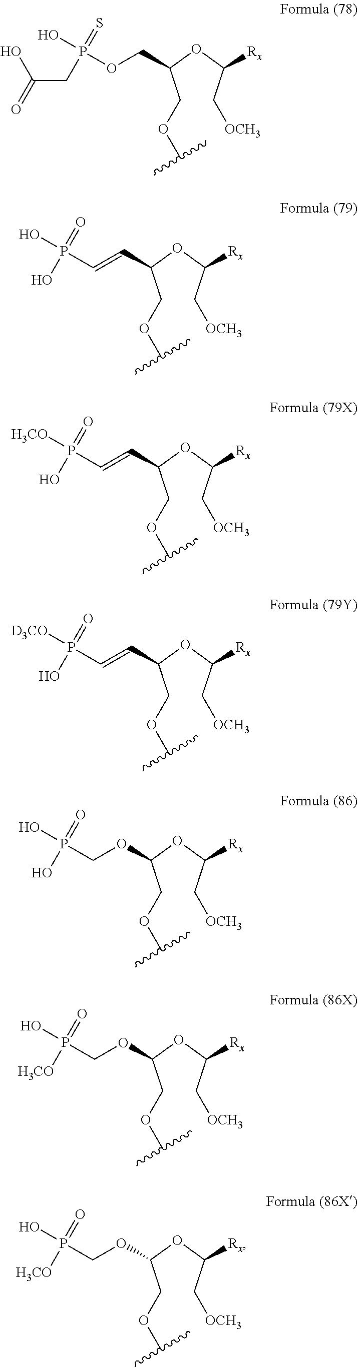

wherein Rₓ is a nucleobase, aryl, heteroaryl, or H.

In some embodiments, any of the siNA molecules disclosed herein comprise a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY):

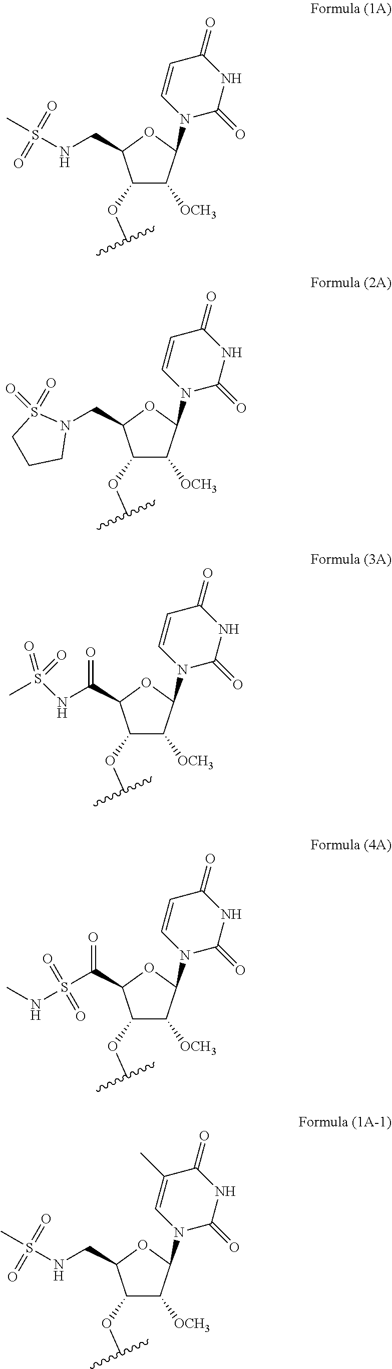 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1C(=O)S(=O)(=O)NC ; *O[C@H]1[C@@H](OC)[C@H](n2cc(C)c(=O)[nH]c2=O)O[C@@H]1CNS(C)(=O)=O]

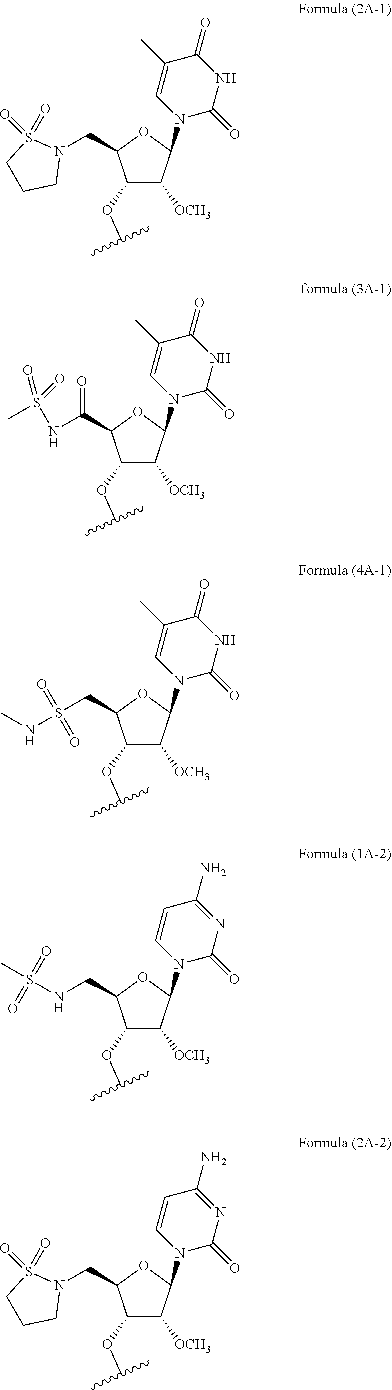 [SMILES: *O[C@H]1[C@@H](OC)[C@H](n2cc(C)c(=O)[nH]c2=O)O[C@@H]1CN1CCCS1(=O)=O ; *O[C@H]1[C@@H](OC)[C@H](n2cc(C)c(=O)[nH]c2=O)O[C@@H]1C(=O)NS(C)(=O)=O ; *O[C@H]1[C@@H](OC)[C@H](n2cc(C)c(=O)[nH]c2=O)O[C@@H]1CS(=O)(=O)NC ; *O[C@H]1[C@@H](OC)[C@H](n2ccc(N)nc2=O)O[C@@H]1CNS(C)(=O)=O ; *O[C@H]1[C@@H](OC)[C@H](n2ccc(N)nc2=O)O[C@@H]1CN1CCCS1(=O)=O]

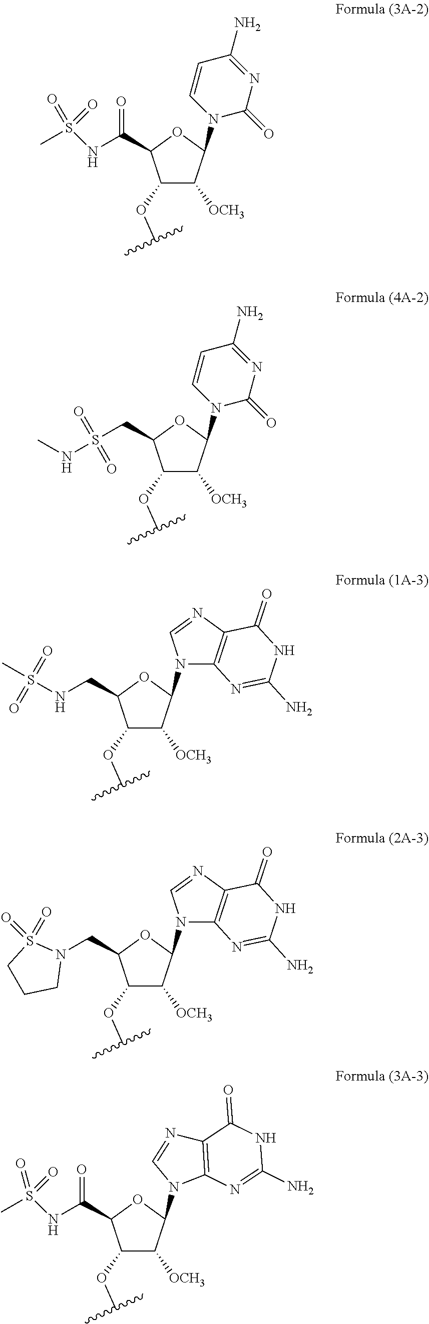 [SMILES: *O[C@H]1[C@@H](OC)[C@H](n2ccc(N)nc2=O)O[C@@H]1C(=O)NS(C)(=O)=O ; *O[C@H]1[C@@H](OC)[C@H](n2ccc(N)nc2=O)O[C@@H]1CS(=O)(=O)NC ; *O[C@H]1[C@@H](OC)[C@H](n2cnc3c(=O)[nH]c(N)nc32)O[C@@H]1CNS(C)(=O)=O ; *O[C@H]1[C@@H](OC)[C@H](n2cnc3c(=O)[nH]c(N)nc32)O[C@@H]1CN1CCCS1(=O)=O ; *O[C@H]1[C@@H](OC)[C@H](n2cnc3c(=O)[nH]c(N)nc32)O[C@@H]1C(=O)NS(C)(=O)=O]

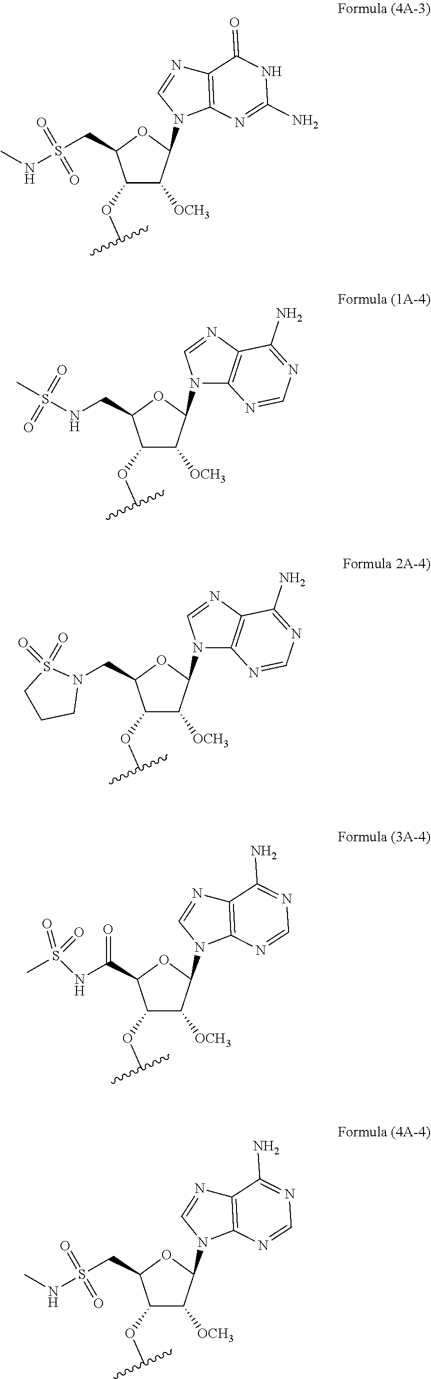 [SMILES: *O[C@H]1[C@@H](OC)[C@H](n2cnc3c(=O)[nH]c(N)nc32)O[C@@H]1CS(=O)(=O)NC ; *O[C@H]1[C@@H](OC)[C@H](n2cnc3c(N)ncnc32)O[C@@H]1CNS(C)(=O)=O ; *O[C@H]1[C@@H](OC)[C@H](n2cnc3c(N)ncnc32)O[C@@H]1CN1CCCS1(=O)=O ; *O[C@H]1[C@@H](OC)[C@H](n2cnc3c(N)ncnc32)O[C@@H]1C(=O)NS(C)(=O)=O ; *O[C@H]1[C@@H](OC)[C@H](n2cnc3c(N)ncnc32)O[C@@H]1CS(=O)(=O)NC]

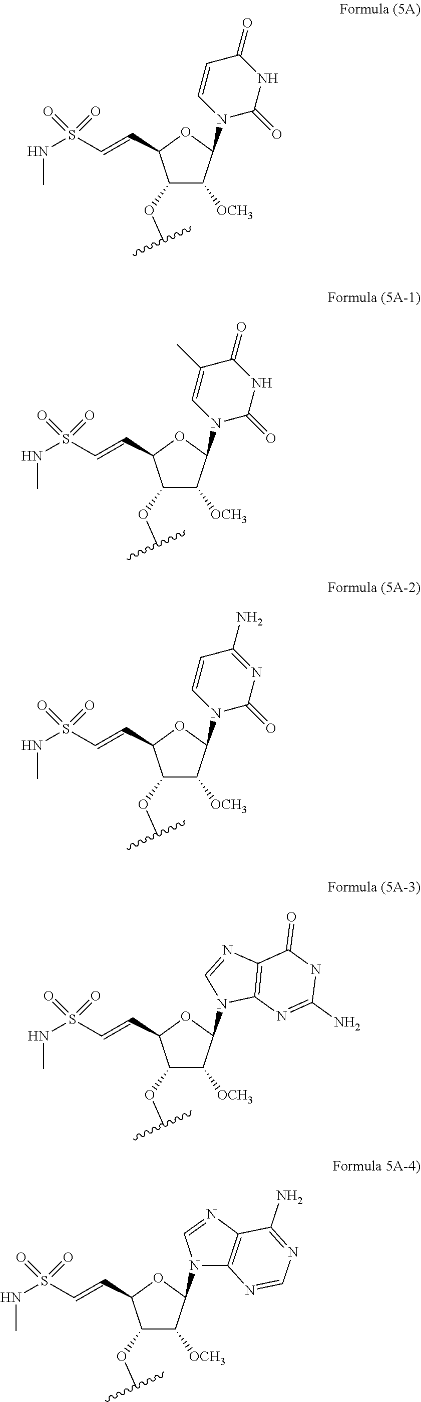 [SMILES: *O[C@H]1[C@@H](OC)[C@H](n2ccc(=O)[nH]c2=O)O[C@@H]1/C=C/S(=O)(=O)NC ; *O[C@H]1[C@@H](OC)[C@H](n2cc(C)c(=O)[nH]c2=O)O[C@@H]1/C=C/S(=O)(=O)NC ; *O[C@H]1[C@@H](OC)[C@H](n2ccc(N)nc2=O)O[C@@H]1/C=C/S(=O)(=O)NC ; *O[C@H]1[C@@H](OC)[C@H](n2cnc3c(=O)[nH]c(N)nc32)O[C@@H]1/C=C/S(=O)(=O)NC ; *O[C@H]1[C@@H](OC)[C@H](n2cnc3c(N)ncnc32)O[C@@H]1/C=C/S(=O)(=O)NC]

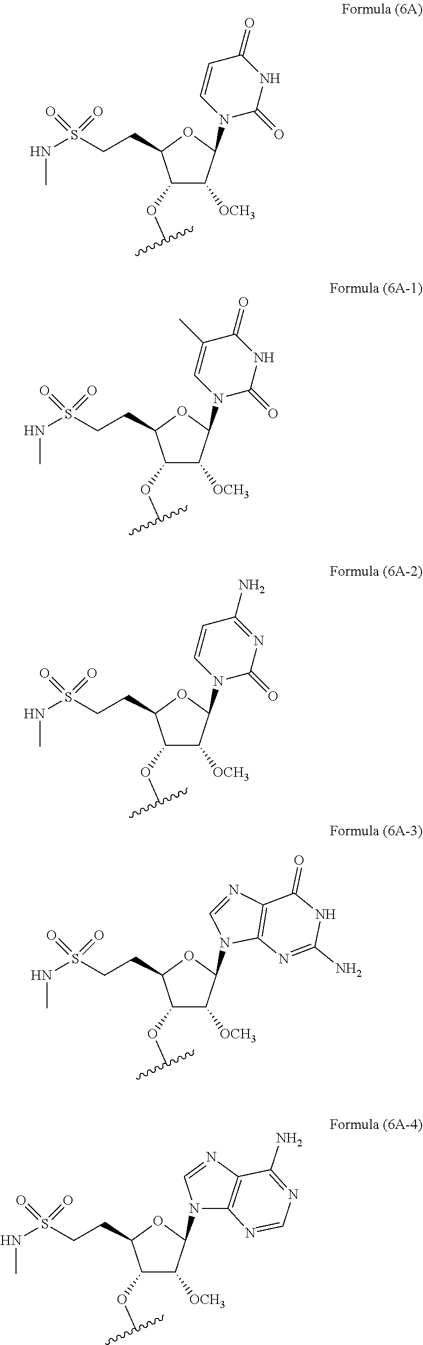 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2cc(C)c(=O)[nH]c2=O)O[C@@H]1CCS(=O)(=O)NC ; *O[C@H]1[C@@H](OC)[C@H](n2ccc(N)nc2=O)O[C@@H]1CCS(=O)(=O)NC ; *O[C@H]1[C@@H](OC)[C@H](n2cnc3c(=O)[nH]c(N)nc32)O[C@@H]1CCS(=O)(=O)NC ; *O[C@H]1[C@@H](OC)[C@H](n2cnc3c(N)ncnc32)O[C@@H]1CCS(=O)(=O)NC]

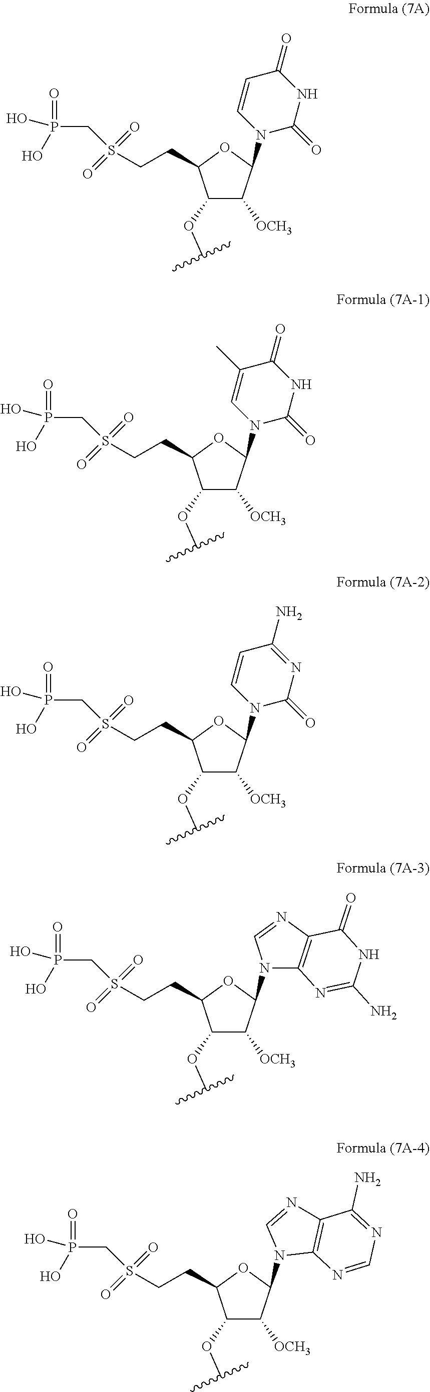 [SMILES: *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2cc(C)c(=O)[nH]c2=O)O[C@@H]1CCS(=O)(=O)CP(=O)(O)O ; *O[C@H]1[C@@H](OC)[C@H](n2ccc(N)nc2=O)O[C@@H]1CCS(=O)(=O)CP(=O)(O)O ; *O[C@H]1[C@@H](OC)[C@H](n2cnc3c(=O)[nH]c(N)nc32)O[C@@H]1CCS(=O)(=O)CP(=O)(O)O ; *O[C@H]1[C@@H](OC)[C@H](n2cnc3c(N)ncnc32)O[C@@H]1CCS(=O)(=O)CP(=O)(O)O]

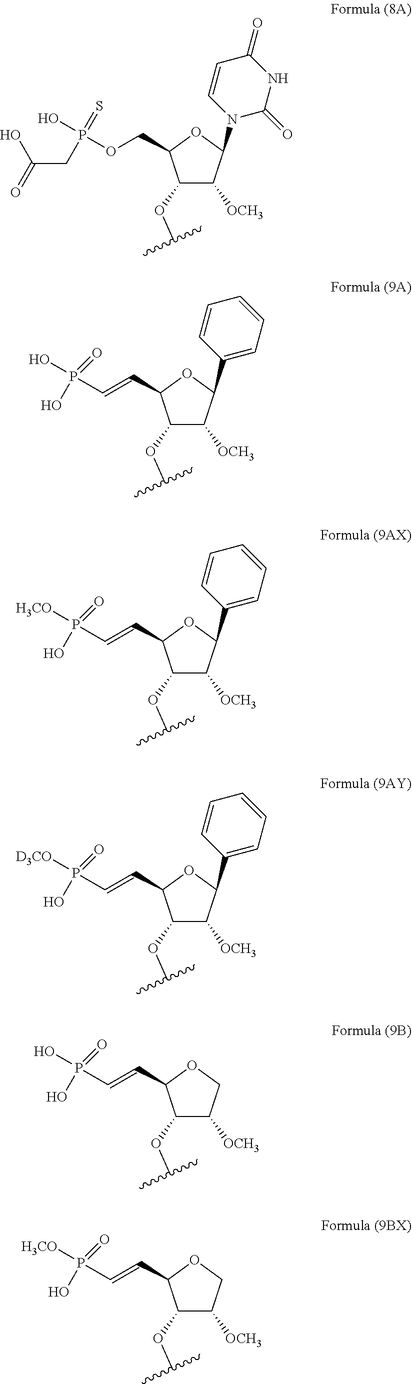

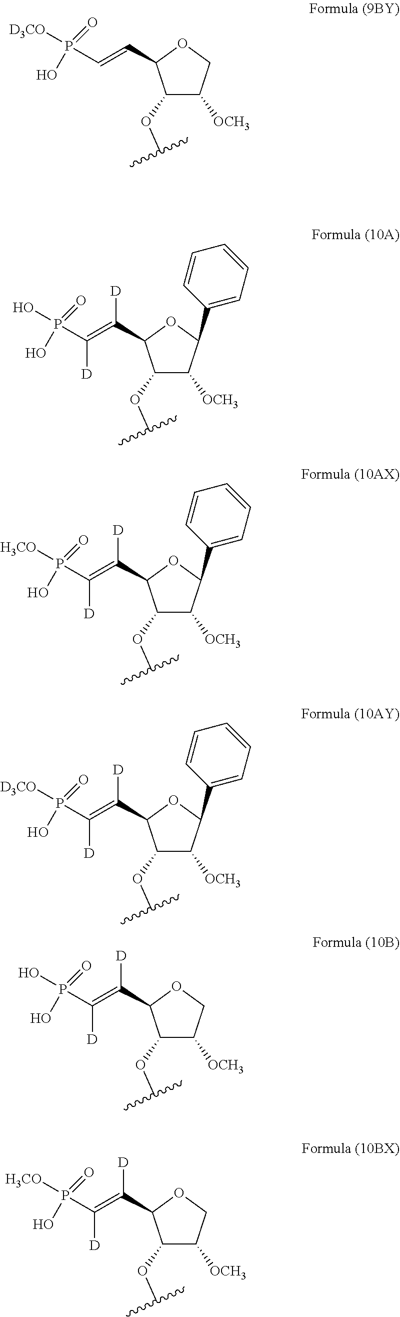

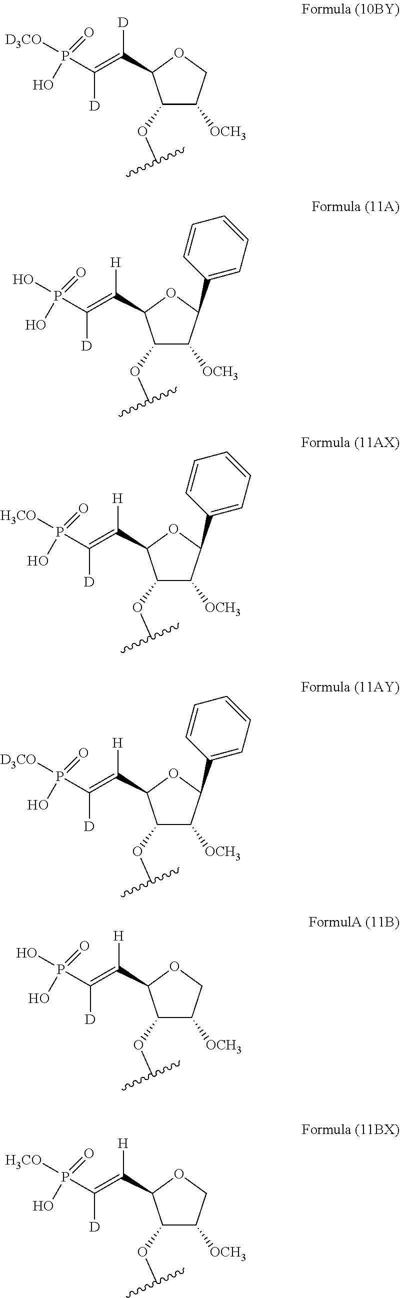

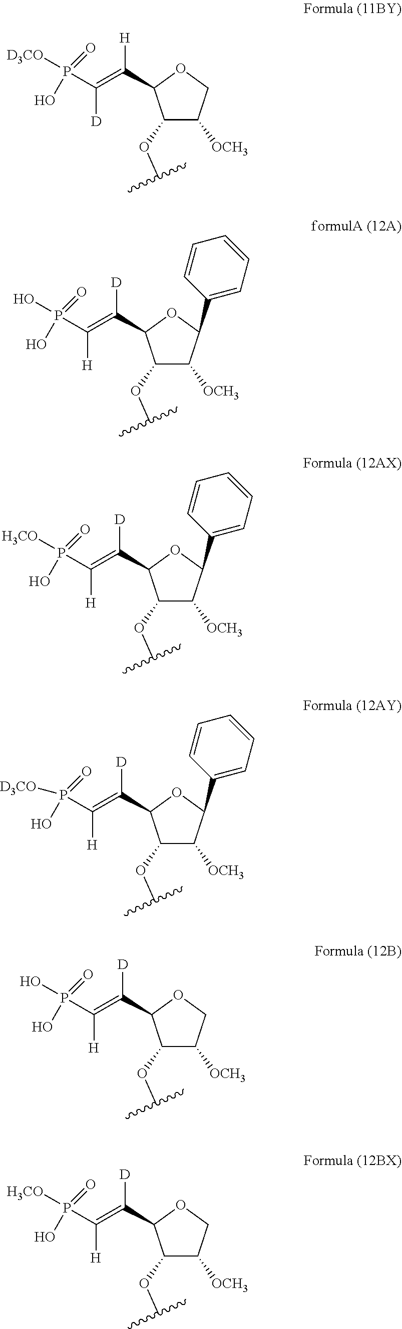

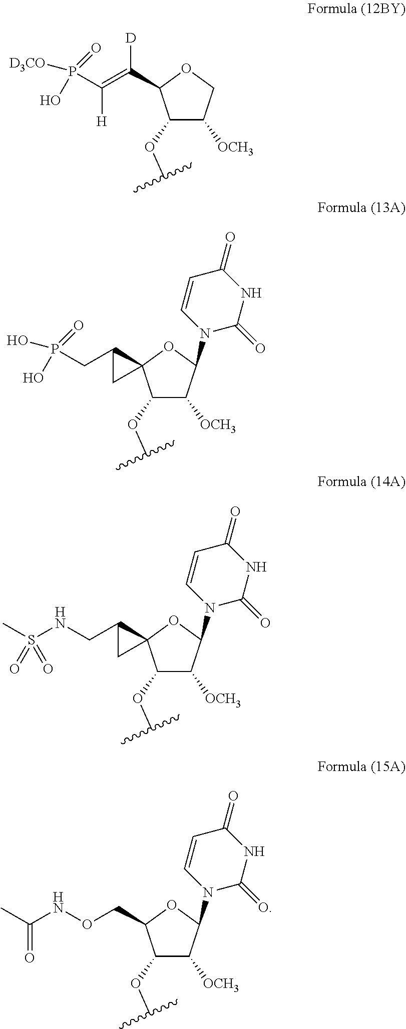

In some embodiments, any of the siNA molecules disclosed herein comprise a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

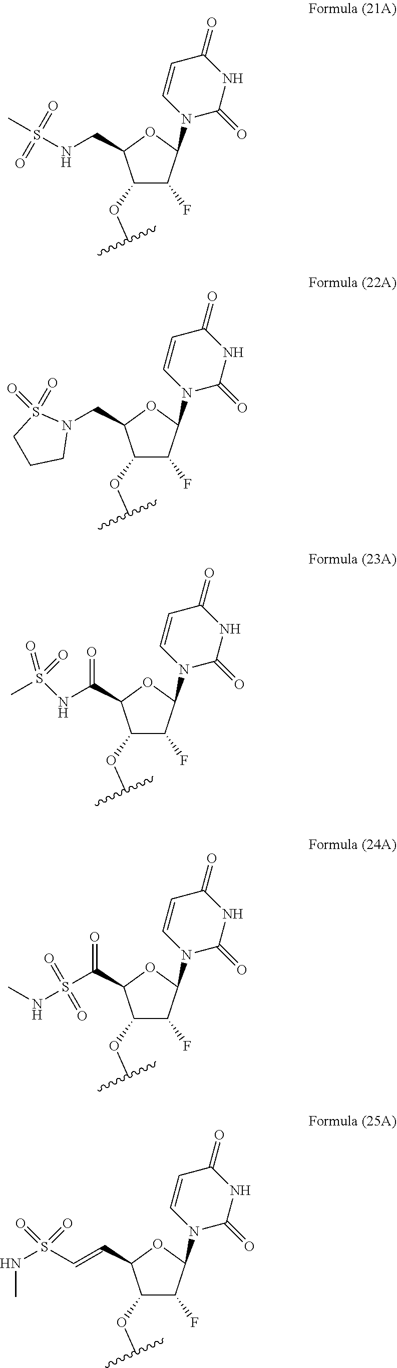 [SMILES: *O[C@H]1[C@@H](F)[C@H](n2ccc(=O)[nH]c2=O)O[C@@H]1CNS(C)(=O)=O ; *O[C@H]1[C@@H](F)[C@H](n2ccc(=O)[nH]c2=O)O[C@@H]1CN1CCCS1(=O)=O ; *O[C@H]1[C@@H](F)[C@H](n2ccc(=O)[nH]c2=O)O[C@@H]1C(=O)NS(C)(=O)=O ; *O[C@H]1[C@@H](F)[C@H](n2ccc(=O)[nH]c2=O)O[C@@H]1C(=O)S(=O)(=O)NC ; *O[C@H]1[C@@H](F)[C@H](n2ccc(=O)[nH]c2=O)O[C@@H]1/C=C/S(=O)(=O)NC]

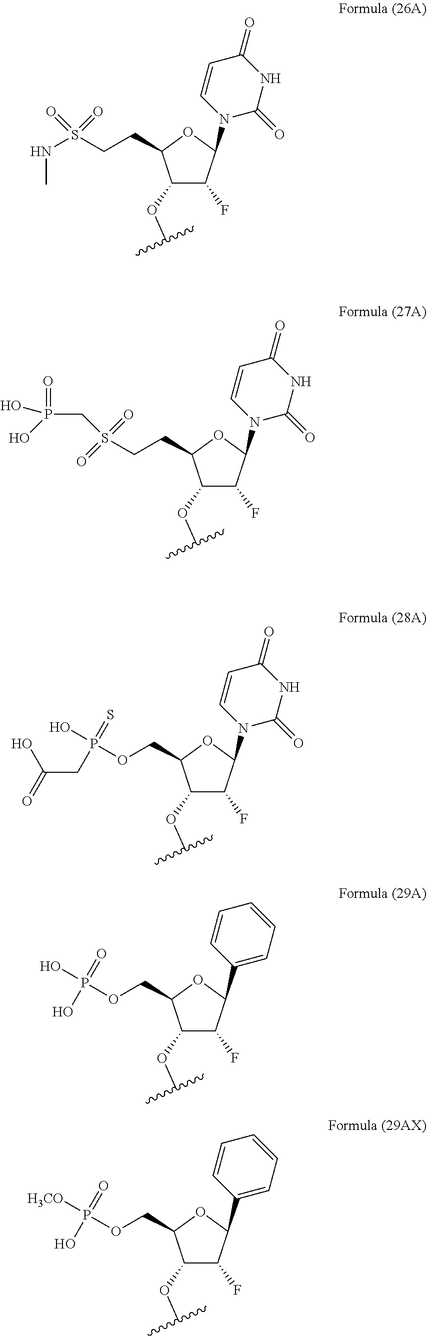 [SMILES: *O[C@H]1[C@@H](F)[C@H](n2ccc(=O)[nH]c2=O)O[C@@H]1CCS(=O)(=O)NC ; *O[C@H]1[C@@H](F)[C@H](n2ccc(=O)[nH]c2=O)O[C@@H]1CCS(=O)(=O)CP(=O)(O)O ; *O[C@H]1[C@@H](F)[C@H](n2ccc(=O)[nH]c2=O)O[C@@H]1COP(O)(=S)CC(=O)O ; *O[C@H]1[C@@H](F)[C@H](c2ccccc2)O[C@@H]1COP(=O)(O)O ; *O[C@H]1[C@@H](F)[C@H](c2ccccc2)O[C@@H]1COP(=O)(O)OC]

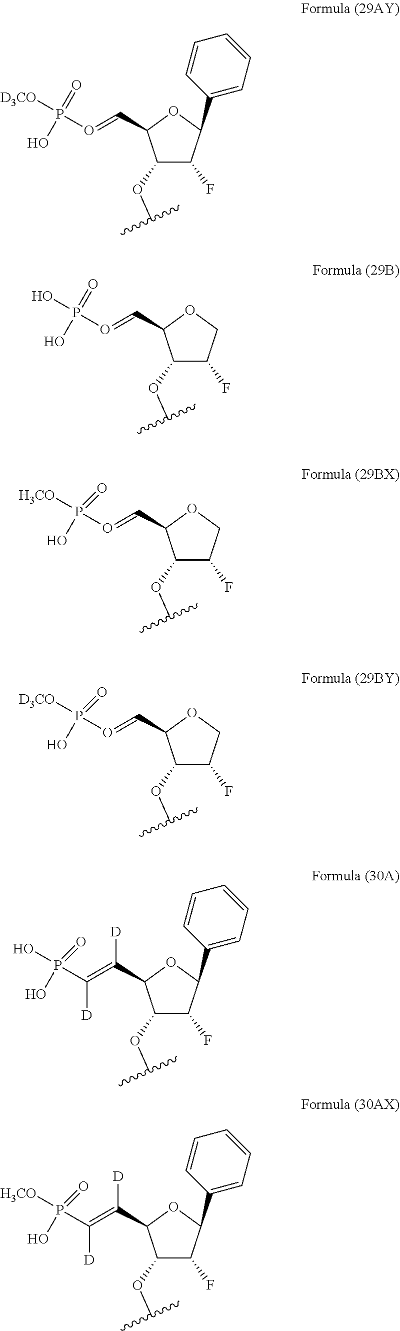

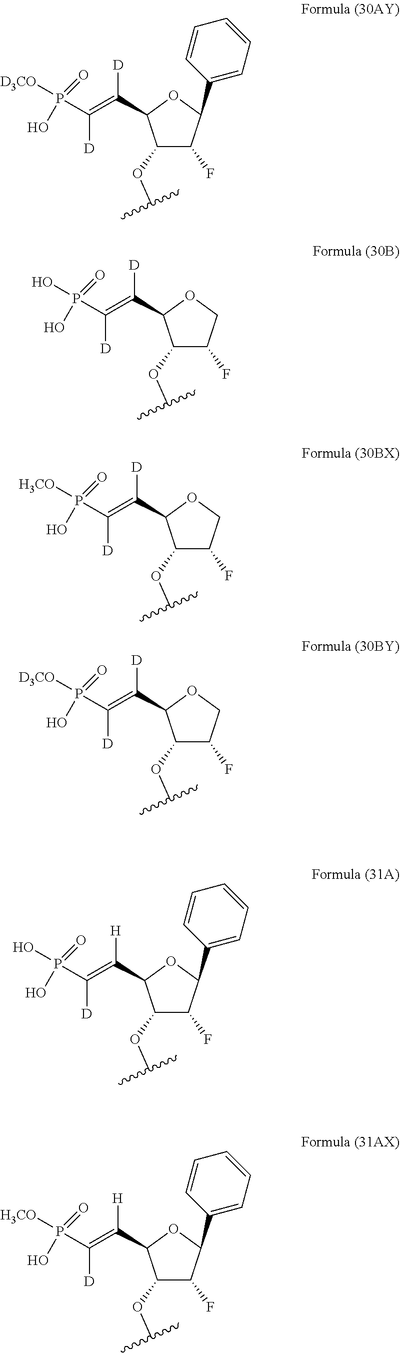

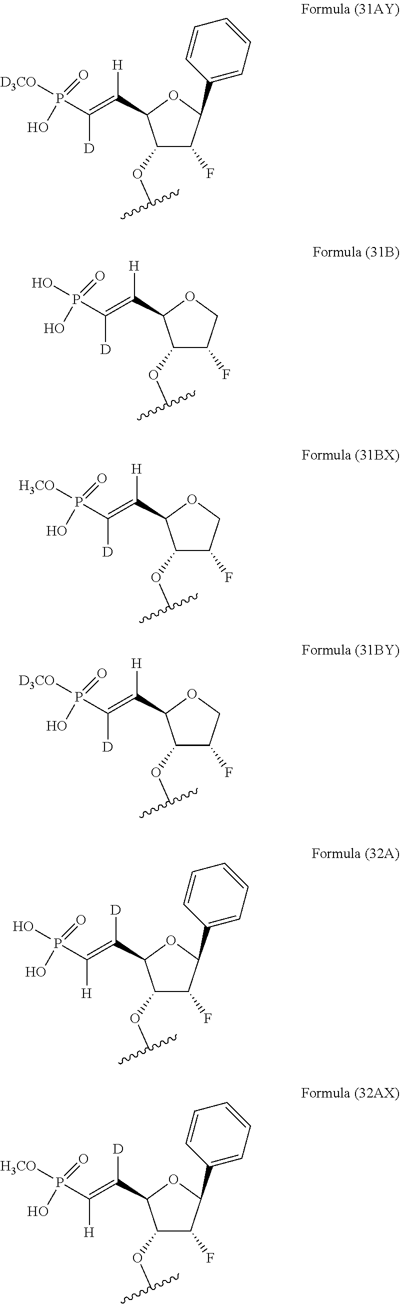

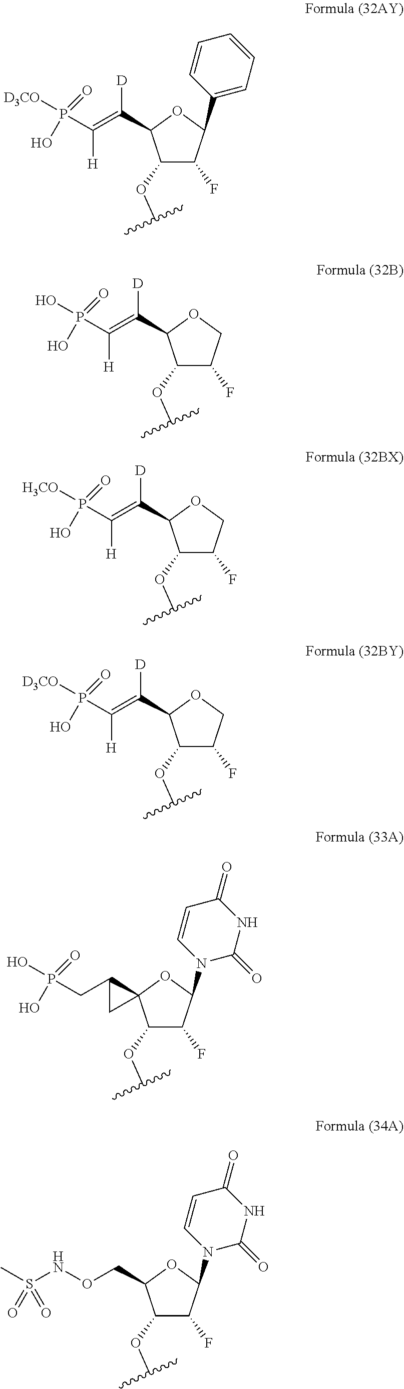

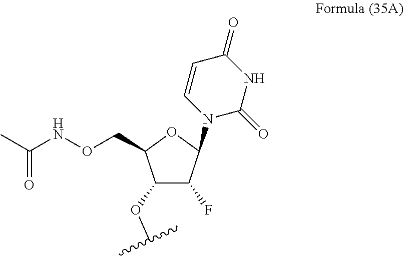 [SMILES: *O[C@H]1[C@@H](F)[C@H](n2ccc(=O)[nH]c2=O)O[C@@H]1CONC(C)=O]

In some embodiments, any of the siNA molecules disclosed herein comprise a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′):

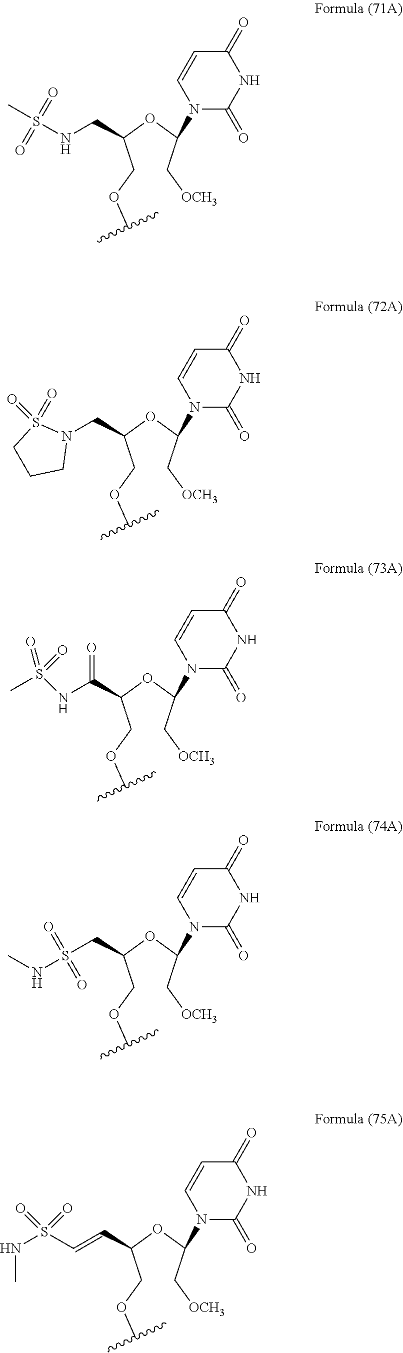 [SMILES: *OC[C@@H](CNS(C)(=O)=O)O[C@H](COC)n1ccc(=O)[nH]c1=O ; *OC[C@@H](CN1CCCS1(=O)=O)O[C@H](COC)n1ccc(=O)[nH]c1=O ; *OC[C@H](O[C@H](COC)n1ccc(=O)[nH]c1=O)C(=O)NS(C)(=O)=O ; *OC[C@@H](CS(=O)(=O)NC)O[C@H](COC)n1ccc(=O)[nH]c1=O ; *OC[C@@H](/C=C/S(=O)(=O)NC)O[C@H](COC)n1ccc(=O)[nH]c1=O]

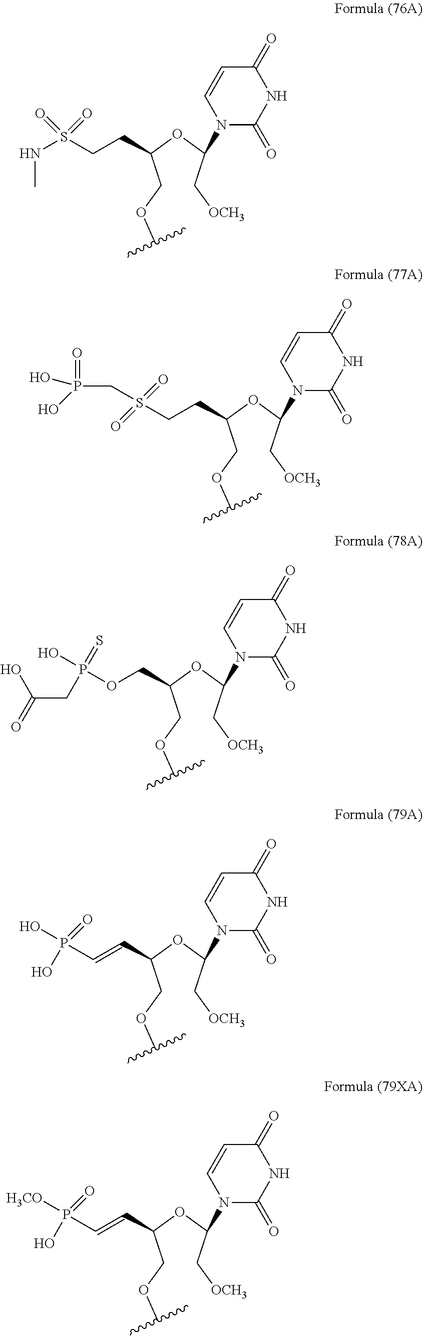 [SMILES: *OC[C@@H](CCS(=O)(=O)NC)O[C@H](COC)n1ccc(=O)[nH]c1=O ; *OC[C@@H](CCS(=O)(=O)CP(=O)(O)O)O[C@H](COC)n1ccc(=O)[nH]c1=O ; *OC[C@@H](COP(O)(=S)CC(=O)O)O[C@H](COC)n1ccc(=O)[nH]c1=O ; *OC[C@@H](/C=C/P(=O)(O)O)O[C@H](COC)n1ccc(=O)[nH]c1=O ; *OC[C@@H](/C=C/P(=O)(O)OC)O[C@H](COC)n1ccc(=O)[nH]c1=O]

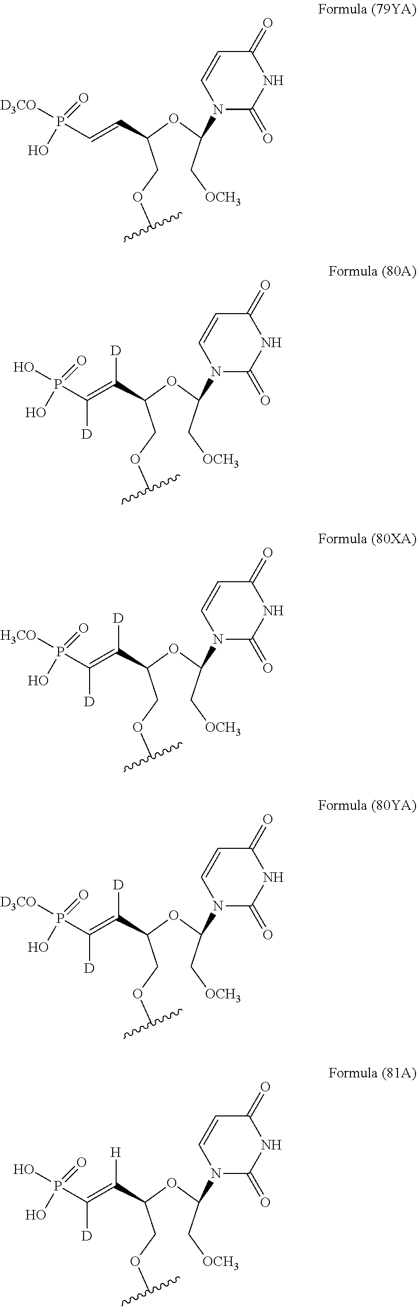

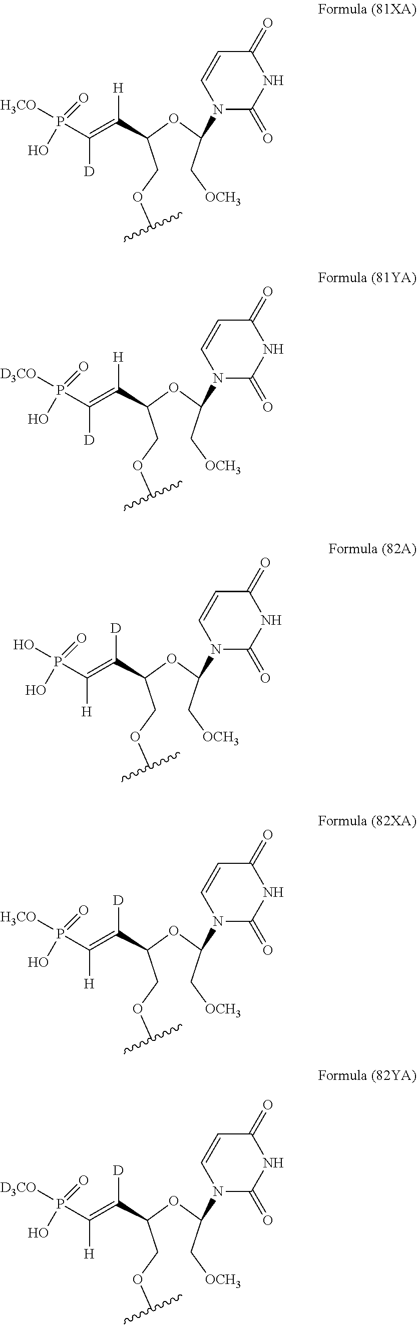

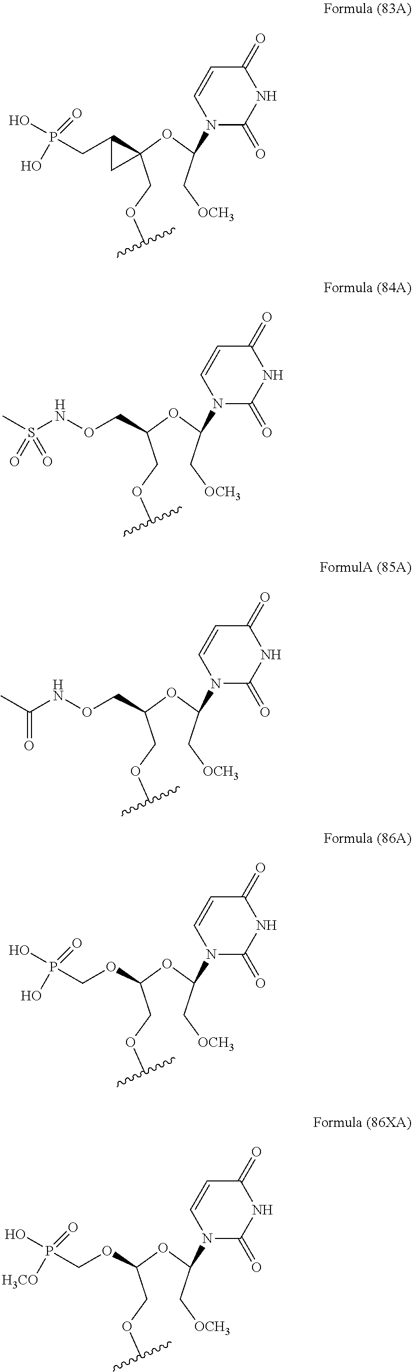

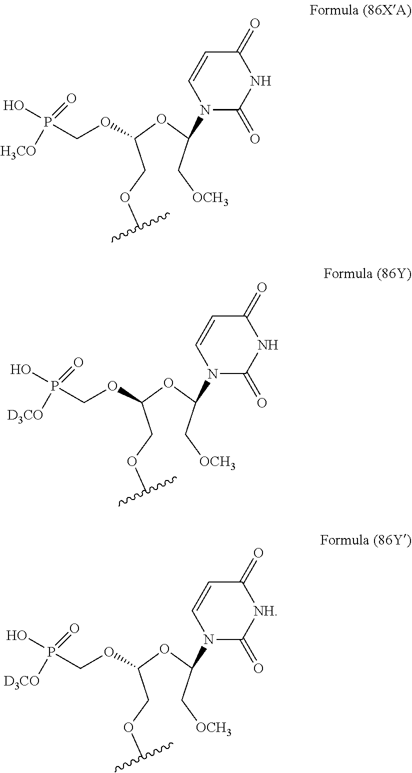

In some embodiments, any of the siNA molecules disclosed herein comprise a 5′-stabilized end cap selected from the group consisting of Formula (78A), Formula (79A), Formula (79XA), Formula (79YA), Formula (86A), Formula (86XA), and Formula (86X′A):

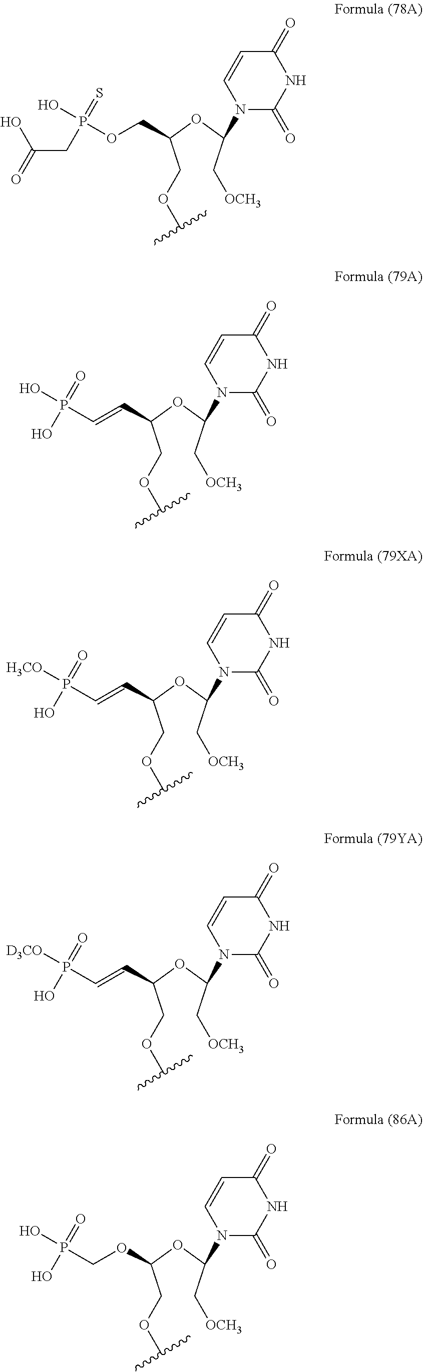

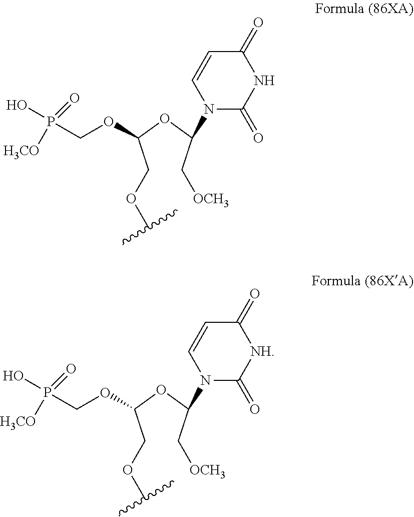 [SMILES: *OC[C@@H](OCP(=O)(O)OC)O[C@H](COC)n1ccc(=O)[nH]c1=O ; *OC[C@H](OCP(=O)(O)OC)O[C@H](COC)n1ccc(=O)[nH]c1=O]

In some embodiments, the 5′-stabilized end cap is attached to the 5′ end of the antisense strand. In some embodiments, the 5′-stabilized end cap is attached to the 5′ end of the antisense strand via 1, 2, 3, 4, or 5 or more linkers. In some embodiments, the one or more linkers are independently selected from the group consisting of a phosphodiester (p or po) linker, phosphorothioate (ps) linker, mesyl phosphoramidate (Ms) linker, phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

As indicated above, the present disclosure provides compositions comprising any of the siNA molecules, sense strands, antisense strands, first nucleotide sequences, or second nucleotide sequences described herein. The disclosed siNA and compositions thereof can be used in the treatment of various diseases and conditions (e.g., viral diseases, liver disease, etc.).

### Linker

In some embodiments, any of the siNAs, sense strands, first nucleotide sequences, antisense strands, and/or second nucleotide sequences disclosed herein comprise 1, 2, 3, 4, 5, 6, 7, 8, 9, 10, 11, 12, 13, 14, 15, 16, 17, 18, 19, 20, 21, 22, 23 or more internucleoside linkers. In some embodiments, 1, 2, 3, 4, 5, 6, 7, 8, 9, 10 or more internucleoside linkers are independently selected from the group consisting of a phosphodiester (p or po) linker, phosphorothioate (ps) linker, mesyl phosphoramidate (Ms) linker, or phosphorodithioate linker.

In some embodiments, any of the siNAs, sense strands, first nucleotide sequences, antisense strands, and/or second nucleotide sequences disclosed herein further comprise 1, 2, 3, 4 or more linkers that attach a conjugated moiety, phosphorylation blocker, and/or 5′ end cap to the siNA, sense strand, first nucleotide sequence, antisense strand, and/or second nucleotide sequences. In some embodiments, the 1, 2, 3, 4 or more linkers are independently selected from the group consisting of a phosphodiester (p or po) linker, phosphorothioate (ps) linker, mesyl phosphoramidate (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

### Target Gene

Without wishing to be bound by theory, upon entry into a cell, any of the ds-siNA molecules disclosed herein may interact with proteins in the cell to form a RNA-Induced Silencing Complex (RISC). Once the ds-siNA is part of the RISC, the ds-siNA may be unwound to form a single-stranded siNA (ss-siNA). The ss-siNA may comprise the antisense strand of the ds-siNA. The antisense strand may bind to a complementary messenger RNA (mRNA), which results in silencing of the gene that encodes the mRNA.

The target gene may be any hydroxysteroid dehydrogenase gene. In any embodiment, the gene is hydroxysteroid 17-beta dehydrogenase 13 (HSD17B13). The HSD17B13 has a sequence shown in the nucleotide sequence of SEQ ID NO: 261, which corresponds to the nucleotide sequence of the coding sequence of GenBank Accession No. NM_178135.5 (nucleotides 42 to 944), which is incorporated by reference in its entirety.

In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within SEQ ID NO: 261.

In some embodiments, the first nucleotide is at least about 60%, 65%, 70%, 75%, 80%, 85%, 90%, 95%, or 100% identical to a nucleotide region within SEQ ID NO: 261, with the exception that the thymines (Ts) in SEQ ID NO: 261 are replaced with uracil (U). In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within SEQ ID NO: 261.

### Compositions

As indicated above, the present disclosure provides compositions comprising any of the siNA molecules, sense strands, antisense strands, first nucleotide sequences, or second nucleotide sequences described herein. The compositions may comprise 1, 2, 3, 4, 5, 6, 7, 8, 9, 10, 11, 12 or more siNA molecules described herein. The compositions may comprise a first nucleotide sequence comprising a nucleotide sequence of any one SEQ ID NOs: 1-100, 201-230, 262-287, 314, and 315. In some embodiments, the composition comprises a second nucleotide sequence comprising a nucleotide sequence of any one of SEQ ID NOs: 101-200, 231-260, and 288-313. In some embodiments, the composition comprises a sense strand comprising a nucleotide sequence of any one of SEQ ID NOs: 1-100, 201-230, 262-287, 314, or 315. In some embodiments, the composition comprises an antisense strand comprising a nucleotide sequence of any one of SEQ ID NOs: 101-200, 231-260, or 288-313.

Alternatively, the compositions may comprise (a) a phosphorylation blocker; and (b) a short interfering nucleic acid (siNA). In some embodiments, the phosphorylation blocker is any of the phosphorylation blockers disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O methyl nucleotide. In some embodiments, the 2′-fluoro nucleotide or the 2′-O methyl nucleotide is independently selected from any of the 2′-fluoro or 2′-O methyl nucleotide mimics disclosed herein. In some embodiments, the siNA comprises a nucleotide sequence comprising any of the modification patterns disclosed herein.

In some embodiments, the composition comprises (a) a conjugated moiety; and (b) a short interfering nucleic acid (siNA). In some embodiments, the conjugated moiety is any of the galactosamines disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O methyl nucleotide. In some embodiments, the 2′-fluoro nucleotide or the 2′-O methyl nucleotide is independently selected from any of the 2′-fluoro or 2′-O methyl nucleotide mimics disclosed herein. In some embodiments, the siNA comprises a nucleotide sequence comprising any of the modification patterns disclosed herein.

In some embodiments, the composition comprises (a) a 5′-stabilized end cap; and (b) a short interfering nucleic acid (siNA). In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O methyl nucleotide. In some embodiments, the 2′-fluoro nucleotide or the 2′-O methyl nucleotide is independently selected from any of the 2′-fluoro or 2′-O methyl nucleotide mimics disclosed herein. In some embodiments, the siNA comprises a nucleotide sequence comprising any of the modification patterns disclosed herein.

In some embodiments, the composition comprises (a) at least one phosphorylation blocker, conjugated moiety, or 5′-stabilized end cap; and (b) a short interfering nucleic acid (siNA). In some embodiments, the phosphorylation blocker is any of the phosphorylation blockers disclosed herein. In some embodiments, the conjugated moiety is any of the galactosamines disclosed herein. In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O methyl nucleotide. In some embodiments, the 2′-fluoro nucleotide or the 2′-O methyl nucleotide is independently selected from any of the 2′-fluoro or 2′-O methyl nucleotide mimics disclosed herein. In some embodiments, the siNA comprises a nucleotide sequence comprising any of the modification patterns disclosed herein.

The composition may be a pharmaceutical composition. In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The composition may be a pharmaceutical composition. In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a siNA of the present disclosure which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., siNA molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siNA molecule) of the present disclosure. In certain embodiments, an aforementioned formulation renders orally bioavailable a compound (e.g., siNA molecule) of the present disclosure.

Methods of preparing these formulations or compositions include the step of bringing into association a compound (e.g., siNA molecule) of the present disclosure with the carrier and, optionally, one or more accessory ingredients. In general, the formulations are prepared by uniformly and intimately bringing into association a compound (e.g., siNA molecule) of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound (e.g., siNA molecule) of the present disclosure as an active ingredient. A compound (e.g., siNA molecule) of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds (e.g., siNA molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds (e.g., siNA molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., siNA molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., siNA molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., siNA molecule) of this disclosure include powders, sprays, ointments, pastes, creams, lotions, gels, solutions, patches and inhalants. The active compound (e.g., siNA molecule) may be mixed under sterile conditions with a pharmaceutically acceptable carrier, and with any preservatives, buffers, or propellants which may be required.

The ointments, pastes, creams and gels may contain, in addition to an active compound (e.g., siNA molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., siNA molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., siNA molecule) of the present disclosure to the body. Such dosage forms can be made by dissolving or dispersing the compound (e.g., siNA molecule) in the proper medium. Absorption enhancers can also be used to increase the flux of the compound (e.g., siNA molecule) across the skin. The rate of such flux can be controlled by either providing a rate controlling membrane or dispersing the compound (e.g., siNA molecule) in a polymer matrix or gel.

Ophthalmic formulations, eye ointments, powders, solutions and the like, are also contemplated as being within the scope of this invention.

Pharmaceutical compositions of this disclosure suitable for parenteral administration comprise one or more compounds (e.g., siNA molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., siNA molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., siNA molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### Treatments and Administration

The siNA molecules of the present disclosure may be used to treat a disease in a subject in need thereof. In some embodiments, a method of treating a disease in a subject in need thereof comprises administering to the subject any of the siNA molecules disclosed herein. In some embodiments, a method of treating a disease in a subject in need thereof comprises administering to the subject any of the compositions disclosed herein.

The preparations (e.g., siNA molecules or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNA molecules) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNA molecules) of the disclosure employed in the pharmaceutical composition at levels lower than that required in order to achieve the desired therapeutic effect and gradually increase the dosage until the desired effect is achieved.

In general, a suitable daily dose of a compound (e.g., siNA molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

When the compounds (e.g., siNA molecules) described herein are co-administered with another, the effective amount may be less than when the compound is used alone.

If desired, the effective daily dose of the active compound (e.g., siNA molecule) may be administered as two, three, four, five, six or more sub-doses administered separately at appropriate intervals throughout the day, optionally, in unit dosage forms. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered once every 1, 2, 3, 4, 5, 6, 7, or 8 weeks.

### Diseases

The siNA molecules and compositions described herein may be administered to a subject to treat a disease. Further disclosed herein are uses of any of the siNA molecules or compositions disclosed herein in the manufacture of a medicament for treating a disease.

In any embodiment, the disease is a liver disease. In any embodiment, the liver disease is nonalcoholic fatty liver disease (NAFLD). In some embodiments, the NAFLD is nonalcoholic steatohepatitis (NASH). In some embodiments, the liver disease is hepatocellular carcinoma (HCC). In some embodiments, the disease is nonalcoholic steatohepatitis (NASH).

Administration of siNA

Administration of any of the siNAs disclosed herein may be conducted by methods known in the art. In some embodiments, the siNA is administered by subcutaneous (SC) or intravenous (IV) delivery. The preparations (e.g., siNAs or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. In some embodiments, subcutaneous administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNAs) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNAs) of the disclosure employed in the pharmaceutical composition at levels lower than that required in order to achieve the desired therapeutic effect and gradually increase the dosage until the desired effect is achieved.

In some embodiments, the siNA or the composition is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg. In some embodiments, the siNA or the composition is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

In some embodiments, the siNA or the composition is administered at least 1, 2, 3, 4, 5, 6, 7, 8, 9, or 10 times. In some embodiments, the siNA or the composition is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month. In some embodiments, the siNA or the composition are administered at least once every 1, 2, 3, 4, 5, 6, 7, 8, 9, 10, 11, 12, 13, 14, 15, 16, 17, 18, 19, 20, or 21 days. In some embodiments, the siNA or the composition is administered for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

In some embodiments, the siNA or the composition is administered at a single dose of 5 mg/kg. In some embodiments, the siNA or the composition is administered at a single dose of 10 mg/kg. In some embodiments, the siNA or the composition is administered at three doses of 10 mg/kg once a week. In some embodiments, the siNA or the composition is administered at three doses of 10 mg/kg once every three days. In some embodiments, the siNA or the composition is administered at five doses of 10 mg/kg once every three days. In some embodiments, the siNA or the composition is administered at six doses of ranging from 1 mg/kg to 15 mg/kg, 1 mg/kg to 10 mg/kg, 2 mg/kg to 15 mg/kg, 2 mg/kg to 10 mg/kg, 3 mg/kg to 15 mg/kg, or 3 mg/kg to 10 mg/kg. In some embodiments, the first dose and second dose are administered at least 3 days apart. In some embodiments, the second dose and third dose are administered at least 4 days apart. In some embodiments, the third dose and fourth dose, fourth dose and fifth dose, or fifth dose and sixth dose are administered at least 7 days apart.

In general, a suitable daily dose of a compound (e.g., siNA) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at about 1 mg/kg to about 40 mg/kg, about 1 mg/kg to about 30 mg/kg, about 1 mg/kg to about 20 mg/kg, about 1 mg/kg to about 15 mg/kg, or 1 mg/kg to about 10 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

If desired, the effective daily dose of the active compound (e.g., siNA) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

The subject of the described methods may be a mammal, and it includes humans and non-human mammals. In some embodiments, the subject is a human, such as an adult human.

### Combination Therapies

Any of the methods disclosed herein may further comprise administering to the subject a liver disease treatment agent. Any of the compositions disclosed herein may further comprise a liver disease treatment agent. In some embodiments, the liver disease treatment agent is selected from a peroxisome proliferator-activator receptor (PPAR) agonist, farnesoid X receptor (FXR) agonist, lipid-altering agent, and incretin-based therapy. In some embodiments, the PPAR agonist is selected from a PPARα agonist, dual PPARα/δ agonist, PPARγ agonist, and dual PPARα/γ agonist. In some embodiments, the dual PPARα agonist is a fibrate. In some embodiments, the PPARα/δ agonist is elafibranor. In some embodiments, the PPARγ agonist is a thiazolidinedione (TZD). In some embodiments, TZD is pioglitazone. In some embodiments, the dual PPARα/γ agonist is saroglitazar. In some embodiments, the FXR agonist is obeticholic acis (OCA). In some embodiments, the lipid-altering agent is aramchol. In some embodiments, the incretin-based therapy is a glucagon-like peptide 1 (GLP-1) receptor agonist or dipeptidyl peptidase 4 (DPP-4) inhibitor. In some embodiments, the GLP-1 receptor agonist is exenatide or liraglutide. In some embodiments, the DPP-4 inhibitor is sitagliptin or vildapliptin. In some embodiments, the siNA and the liver disease treatment agent are administered concurrently. In some embodiments, the siNA and the liver disease treatment agent are administered sequentially. In some embodiments, the siNA is administered prior to administering the liver disease treatment agent. In some embodiments, the siNA is administered after administering the liver disease treatment agent. In some embodiments, the siNA and the liver disease treatment agent are in separate containers. In some embodiments, the siNA and the liver disease treatment agent are in the same container.

### EXAMPLES

The following examples are provided to illustrate the present disclosure. Those ordinarily skilled in the art will readily understand that known variations of the following methods, procedures, and/or materials can be used. These examples are provided for the purpose of further illustration and are not intended to be limitations on the disclosure.

Throughout the disclosure, including in the sequences, abbreviations and acronyms may be used with the following meanings unless otherwise indicated:

| Abbreviation(s) | Reagent |
|---|---|
| A | Adenosine |
| C | Cytidine |
| G | Guanosine |
| U | Uridine |
| fX | 2′-fluoro on X where X is A, C, G, or U |
| mX | 2′-O-methyl on X where X is A, C, G, or |
| | U |
| ps | phosphorothioate internucleoside linkage |
| v | vinyl phosphonate |
| EC50 | half-maximal effective concentration |
| GalNAc | N-acetylgalactosamine (including |
| | variations thereof, such as GalNAc4) |
| PD | pharmacodynamics |
| PK | pharmacokinetics |
| PNPLA3 | Patatin-like phospholipase domain- |
| | containing protein 3 gene, including |
| | variants thereof as described herein |
| RT-qPCR | reverse transcriptase-quantitative |
| | polymerase chain reaction |
| DMF | Dimethylformamide |
| AcSK | Acesulfame potassium |
| TBAI | Tetra-n-butylammonium iodide |
| H₂O | Water |
| EA/EtOAc | Ethyl acetate |
| Na₂SO₄ | Sodium sulfate |
| CDCl₃ | Deuterated chloroform |
| CH₃CN/ACN/MeCN | Acetonitrile |
| MeOH | Methanol |
| NaOH | Sodium hydroxide |
| Ar | Argon gas |
| HCl | Hydrochloric acid |
| i-Pr₂O | Diisopropyl ether |
| THF | Tetrahydrofuran |
| LiBr | Lithium bromide |
| DIEA/DIPEA | N,N-Diisopropylethylamine |
| Pd/C | Palladium metal on carbon support |
| N₂ | Nitrogen gas |
| H₂ | Hydrogen gas |
| CD₃CN | Deuterated acetonitrile |
| TBAF | Tetra-n-butylammonium fluoride |
| DCM/CH₂Cl₂ | Dichloromethane |
| MS | Molecular sieves |
| NaHCO₃ | Sodium bicarbonate |
| NH₄HCO₃ | Ammonium bicarbonate |
| iPrOH/iPr-OH/IPA | Isopropanol |
| TEA | Triethanolamine |
| PPh₃ | Triphenylphosphine |
| DIAD | Diisopropyl azodicarboxylate |
| EtOH | Ethanol |
| NH2NH2•H2O | Hydrazine monohydrate |
| DMSO-d₆ | Deuterated dimethyl sulfoxide |
| Py/Pyr | Pyridine |
| MsCl | Methanesulfonyl chloride |
| PE | Petroleum ether |
| CH₃COOH/AcOH | Acetic acid |
| SiO₂ | Silica/Silicone dioxide |
| I₂ | Iodine |
| Na₂S₂O₃ | Sodium thiosulfate |
| AgNO₃ | Silver nitrate |
| DMTCl/DMTrCl | 4,4′-dimethoxytrityl chloride |
| DTT | Dithiothreitol |
| LiOH•H₂O | Lithium hydroxide monohydrate |
| DCI | 1,l′-Carbonyldiimidazole |
| TEMPO | (2,2,6,6-Tetramethylpiperidin-1-yl)oxyl |
| DIB | Diisobutylene |
| SOCl₂ | Thionyl chloride |
| CD₃OD | Deuterated methanol |
| NaBD₄ | Sodium borodeuteride |
| TBSCl | Tert-butyldimethylsilyl chloride |
| Et3SiH | Triethylsilane |
| TFA | Trifluoroacetic acid |
| NH₃•H₂O/NH₃*H₂O | Ammonia |
| FA/HCOOH/HCO₂H | Formic acid |
| BTT | Benzyl-thio-tetrazole |
| DDTT | 3-[(Dimethylaminomethylene)amino]- |
| | 3H-1,2,4-dithiazole-5-thione |
| K₂CO₃ | Potassium carbonate |
| NaH₂PO₄ | Monosodium phosphate |
| NaBr | Sodium bromide |
| KSAc | Potassium thioacetate |
| LiAlH₄ | Lithium aluminium hydride |
| DMSO | Dimethyl sulfoxide |
| CEOP[N(iPr)₂]₂/ | 2-Cyanoethyl N,N- |
| CEP[N(iPr)₂]₂/CEP/CEPCl | diisopropylchlorophosphoramidite |
| (CD₃O)₂Mg | Deuterated magnesium methoxide or d6- |
| | magnesium methoxide |
| NH₄Cl | Ammonium chloride |
| ACN-d₃ | Deuterated acetonitrile |
| D₂O | Heavy water/deuterium oxide |
| PDC | Pyridinium dichromate |
| Ac₂O | Acetic anhydride |
| MeOD | Monodeuterated methanol |
| CH₃COOD | Monodeuteroacetic acid |
| DCA | Dichloroacetic acid |
| TES | 2-{[1,3-Dihydroxy-2- |
| | (hydroxymethyl)propan-2- |
| | yl]amino}ethane-1-sulfonic acid |
| DMAP | 4-Dimethylaminopyridine |
| TPSCl | Triphenylsilyl chloride |
| BzCl | Benzoyl chloride |
| DMTrSH | 4,4′-Dimethoxytrityl thiol |
| NaOMe | Sodium methoxide |
| EDCI | 1-Ethyl-3-(3- |
| | dimethylaminopropyl)carbodiimide |
| POM | Polyoxymethylene |
| KOH | Potassium hydroxide |
| NaCl | Sodium chloride |
| iBuCl | Isobutyryl chloride |
| DAIB | (Diacetoxyiodo)benzene |
| NaI | Sodium iodide |
| Boc | Tert-butyloxycarbonyl |
| TMG | Tetramethylguanidine |
| TMSCHN₂ | Trimethylsilyldiazomethane |
| IBX | 2-Iodoxybenzoic acid |
| PivCl | Pivaloyl chloride/chloromethyl pivalate |
| NaH | Sodium hydride |
| CD₃I | Iodomethane-d₃ |
| BSA | Bis(trimethylsilyl)acetamide |
| TMSOTf | Trimethylsilyl trifluoromethanesulfonate |
| CH₃NH₂ | Methylamine |
| DPC | 1,5-Diphenylcarbazide |
| TrtCl/TrCl | Trityl chloride |
| DAST | Diethylaminosulfur trifluoride |
| Tf-Cl/TfCl | Trifluoromethanesulfonyl chloride |
| Et₃N | Triethylamine |
| KOAc | Potassium acetate |
| DABCO | 1,4-Diazabicyclo[2.2.2]octane |
| NaOAc | Sodium acetate |
| n-BuLi | n-Butyl lithium |
| BF₃•OEt₂ | Boron trifluoride etherate |
| BCl₃ | Boron trichloride/trichloroborane |
| NaN₃ | Sodium azide |
| DBU | 1,8-Diazabicyclo[5.4.0]undec-7-ene |
| NH₄F | Ammonium fluoride |
| (COCl)₂ | Oxalyl dichloride |
| MeNH₂ | Methylamine |
| Rh₂(OAc)₄ | Rhodium (II) acetate |
| Boc₂O | Di-tert-butyl dicarbonate |
| PPTS | Pyridinium p-toluenesulfonate |
| Ms₂O | Methanesulfonic anhydride |
| NaBH₄ | Sodium borohydride |
| PhCO2K | Potassium benzoate |
| p-TsOH/TsOH | p-Toluenesulfonic acid |
| NH₃ | Ammonia |
| TBDPSCl | tert-Butyldiphenylsilyl chloride |
| NaIO₄ | Sodium periodate |
| BAIB | (Diacetoxyiodo)benzene |
| Pb(OAc)₄ | Lead (IV) tetraacetate |
| MgSO₄ | Magnesium sulfate |
| CO₂ | Carbon dioxide |
| H₂O₂ | Hydrogen peroxide |
| CaCO₃ | Calcium carbonate |
| DIBAL-H | Diisobutylaluminum hydride |
| CuSO₄ | Copper (II) sulfate |
| CH₃I | Iodomethane |
| Ag₂O | Silver oxide |
| SnCl₄ | Tin (IV) chloride |
| MMTrCl | 4-Methoxytrityl chloride |
| Et₃Si | Triethylsilane |
| NaNO₂ | Sodium nitrite |
| TMSCl | Trimethylsilyl chloride |
| PacCl | Phenoxyacetyl chloride |
| BOMCl | Benzyl chloromethyl ether |
| DCE | Ethylene dichloride |
| t-BuOH | T-butyl alcohol |
| P₂O₅ | Phosphorus pentoxide |
| ETT | 5-Ethylthio-1H-tetrazole |
| AMA | Ammonia methylamine |

### Example 1. siNA Synthesis

This example describes an exemplary method for synthesizing ds-siNAs.

The 2′-OMe phosphoramidite 5′-O-DMT-deoxy Adenosine (NH-Bz), 3′-O-(2-cyanoethyl-N,N-diisopropyl phosphoramidite, 5′-O-DMT-deoxy Guanosine (NH-ibu), 3′-O-(2-cyanoethyl-N,N-diisopropyl phosphoramidite, 5′-O-DMT-deoxy Cytosine (NH-Bz), 3′-O-(2-cyanoethyl-N,N-diisopropyl phosphoramidite, 5′-O-DMT-Uridine 3′-O-(2-cyanoethyl-N,N-diisopropyl phosphoramidite were purchased from Thermo Fisher Milwaukee WI, USA.

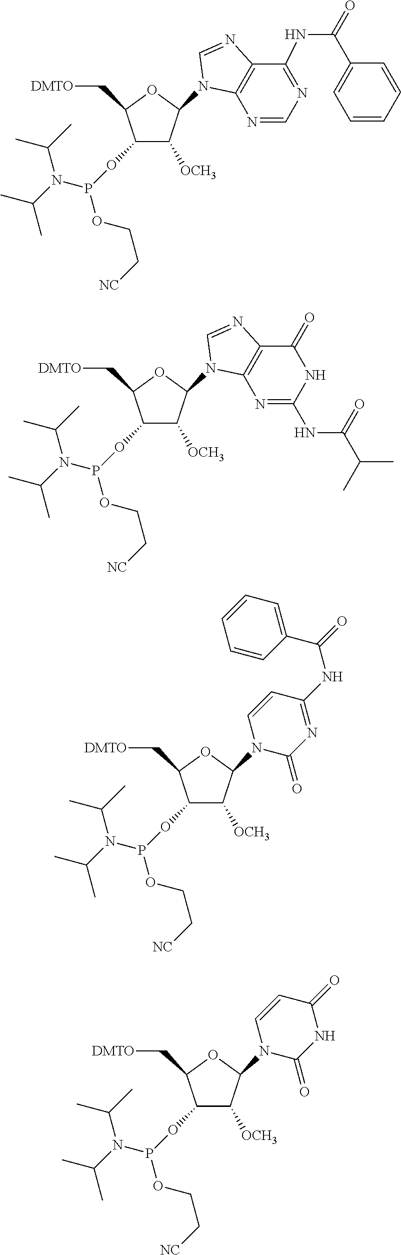

The 2′-F-5′-O-DMT-(NH-Bz) Adenosine-3′-O-(2-cyanoethyl-N,N-diisopropyl phosphoramidite, 2′-F-5′-O-DMT-(NH-ibu)-Guanosine, 3′-O-(2-cyanoethyl-N,N-diisopropyl phosphoramidite, 5′-O-DMT-(NH-Bz)-Cytosine, 2′-F-3′-O-(2-cyanoethyl-N,N-diisopropyl phosphoramidite, 5′-O-DMT-Uridine, 2′-F-3′-O-(2-cyanoethyl-N,N-diisopropyl phosphoramidite were purchased from Thermo Fisher Milwaukee WI, USA.

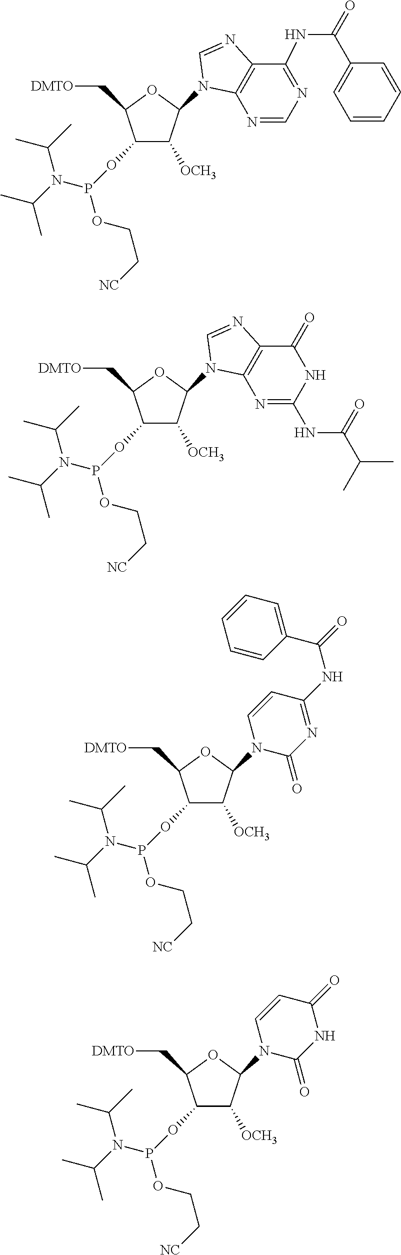

All the monomers were dried in vacuum desiccator with desiccants (P₂O₅, RT 24 h). The solid supports (CPG) attached to the nucleosides and universal supports were obtained from LGC and Chemgenes. The chemicals and solvents for post synthesis workflow were purchased from commercially available sources like VWR/Sigma and used without any purification or treatment. Solvent (Acetonitrile) and solutions (amidite and activator) were stored over molecular sieves during synthesis.

The oligonucleotides were synthesized on DNA/RNA Synthesizers (Expedite 8909 or ABI-394 or MM-48) using standard oligonucleotide phosphoramidite chemistry starting from the 3′ residue of the oligonucleotide preloaded on CPG support. An extended coupling of 0.1M solution of phosphoramidite in CH₃CN in the presence of 5-(ethylthio)-1H-tetrazole activator to a solid bound oligonucleotide followed by standard capping, oxidation and deprotection afforded modified oligonucleotides. The 0.1M I₂, THF: Pyridine; Water-7:2:1 was used as oxidizing agent while DDTT ((dimethylamino-methylidene)amino)-3H-1,2,4-dithiazaoline-3-thione was used as the sulfur-transfer agent for the synthesis of oligoribonucleotide phosphorothioates. The stepwise coupling efficiency of all modified phosphoramidites was more than 98%.

| Reagents | Detailed Description |
|---|---|
| Deblock Solution | 3% Dichloroacetic acid (DCA) in Dichloro- |
| | methane |
| Amidite Concentration | 0.1M in Anhydrous Acetonitrile |
| Activator | 0.25M Ethyl-thio-Tetrazole (ETT) |
| Cap-A solution | Acetic anhydride in Pyridine/THF |
| Cap-B Solution | 16% 1-Methylimidazole in THF |
| Oxidizing Solution | 0.02M I₂, THF:Pyridine; Water-7:2:1 |
| Sulfurizing Solution | 0.2M DDTT in Pyridine/Acetonitrile 1:1 |

### Cleavage and Deprotection:

Deprotection and cleavage from the solid support was achieved with mixture of ammonia methylamine (1:1, AMA) for 15 min at 65° C. When the universal linker was used, the deprotection was left for 90 min at 65° C. or solid supports were heated with aqueous ammonia (28%) solution at 55° C. for 8-16 h to deprotect the base labile protecting groups.

Quantitation of Crude siNA

Samples were dissolved in deionized water (1.0 mL) and quantitated as follows: blanking was first performed with water alone (2 ul) on Thermo Scientific™ Nanodrop UV spectrophotometer or BioTek™ Epoch™ plate reader then oligo sample reading was obtained at 260 nm. The crude material is dried down and stored at −20° C.

### Crude HPLC/LC-MS Analysis

The 0.1 OD of the crude samples were analyzed by HPLC and LC-MS. After confirming the crude LC-MS data then purification step was performed if needed based on the purity.

### HPLC Purification

The unconjugated and GalNAc modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.0 M NaBr, pH 8.5 (buffer B). Fractions containing full-length oligonucleotides were pooled.

Desalting of Purified siNA

The purified dry siNA was then desalted using Sephadex G-25 M (Amersham Biosciences). The cartridge was conditioned with 10 mL of deionized water thrice. Finally, the purified siNA dissolved thoroughly in 2.5 mL RNAse free water was applied to the cartridge drop wise. The salt free siNA was eluted with 3.5 mL deionized water directly into a screw cap vial. Alternatively, some unconjugated siNA was deslated using Pall AcroPrep™ 3K MWCO desalting plates.

### IEX HPLC and Electrospray LC/MS Analysis

Approximately 0.10 OD of siNA was dissolved in water and then pipetted into HPLC autosampler vials for IEX-HPLC and LC/MS analysis. Analytical HPLC and ES LC-MS confirmed the identity and purity of the compounds.

### Duplex Preparation:

Single strand oligonucleotides (Sense and Antisense strands) were annealed (1:1 by molar equivalents, heat at 90° C. for 2 min followed by gradual cooling at room temperature) to give the duplex ds-siNA. The final compounds were analyzed on size exclusion chromatography (SEC).

### Example 2: Synthesis of 5′ End Cap Monomer

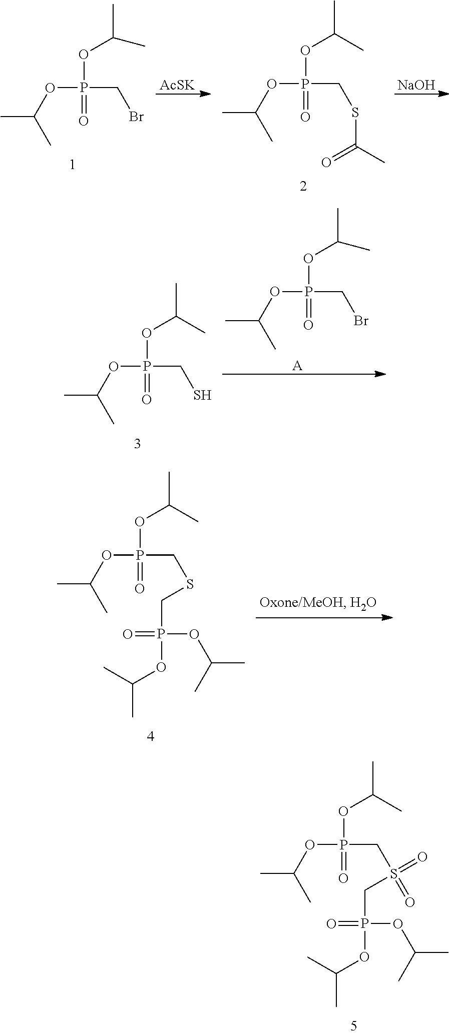

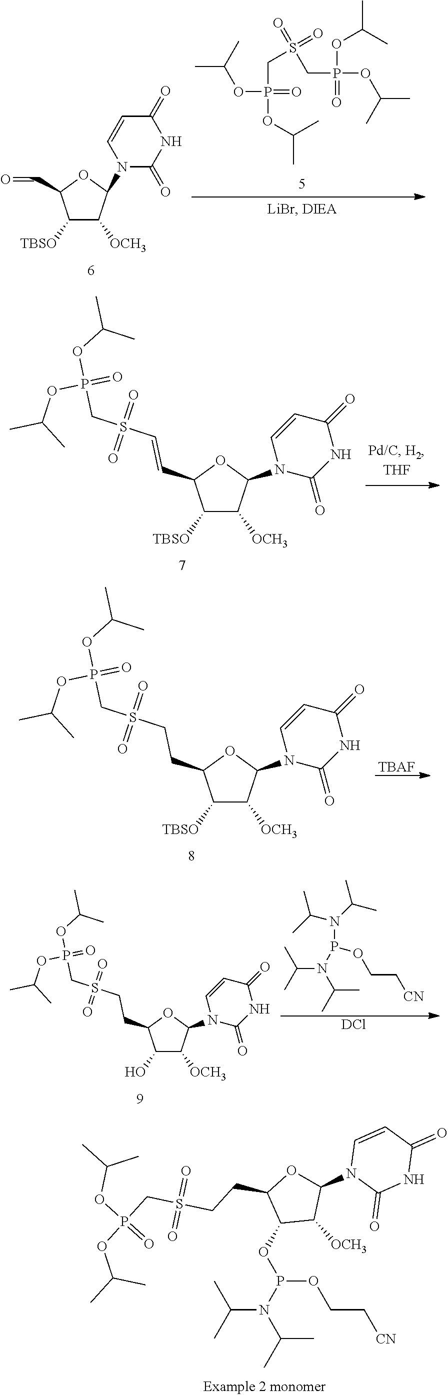

### Example 2 Monomer Synthesis Scheme

Preparation of (2): To a solution of 1 (15 g, 57.90 mmol) in DMF (150 mL) were added AcSK (11.24 g, 98.43 mmol) and TBAI (1.07 g, 2.89 mmol), and the mixture was stirred at 25° C. for 12 h. Upon completion as monitored by LCMS, the mixture was diluted with H₂O (10 mL) and extracted with EA (200 mL*3). The combined organic layers were washed with brine (200 mL*3), dried over anhydrous Na₂SO₄, filtered and concentrated under reduced pressure to give 2 (14.5 g, 96.52% yield, 98% purity) as a colorless oil. ESI-LCMS: 254.28 [M+H]⁺; ¹H NMR (400 MHz, CDCl₃) δ=4.78-4.65 (m, 2H), 3.19 (d, J=14.1 Hz, 2H), 2.38 (s, 3H), 1.32 (t, J=6.7 Hz, 12H); ³¹P NMR (162 MHz, CDCl₃) δ=20.59.

Preparation of (3): To a solution of 2 (14.5 g, 57.02 mmol) in CH₃CN (50 mL) and MeOH (25 mL) was added NaOH (3 M, 28.51 mL), and the mixture was stirred at 25° C. for 12 h under Ar. Upon completion as monitored by TLC, the reaction mixture was concentrated under reduced pressure to remove CH₃CN and CH₃OH. The residue was diluted with water (50 mL) and adjust pH=7 by 6M HCl, and the mixture was extracted with EA (50 mL*3). The combined organic layers were washed with brine (50 mL*3), dried over anhydrous Na₂SO₄, filtered and concentrated under reduced pressure to give 3 (12.1 g, crude) as a colorless oil.

Preparation of (4): To a solution of 3 (12.1 g, 57.01 mmol) in CH₃CN (25 mL) and MeOH (25 mL) was added A (14.77 g, 57.01 mmol) dropwise at 25° C., and the mixture was stirred at 25° C. under Ar for 12 h. Upon completion as monitored by LCMS, the reaction mixture was concentrated under reduced pressure to give 4 (19.5 g, 78.85% yield) as a colorless oil. ¹H NMR (400 MHz, CDCl₃) δ=4.80-4.66 (m, 4H), 2.93 (d, J=11.3 Hz, 4H), 1.31 (dd, J=3.9, 6.1 Hz, 24H); ³¹P NMR (162 MHz, CDCl₃) δ=22.18.

Preparation of (5): To a solution of 4 (19.5 g, 49.95 mmol) in MeOH (100 mL) and H₂O (100 mL) was added Oxone (61.41 g, 99.89 mmol) at 25° C. in portions, and the mixture was stirred at 25° C. for 12 h under Ar. Upon completion as monitored by LCMS, the reaction mixture was filtered, and the filtrate was concentrated under reduced pressure to remove MeOH. The residue was extracted with EA (50 mL*3). The combined organic layers were washed with brine (50 mL*3), dried over anhydrous Na₂SO₄, filtered and concentrated under reduced pressure to give a residue. The crude product was triturated with i-Pr₂O and n-Hexane (1:2, 100 mL) at 25° C. for 30 min to give 5 (15.6 g, 73.94% yield) as a white solid. ¹H NMR (400 MHz, CDCl₃) δ=4.92-4.76 (m, 4H), 4.09 (d, J=16.1 Hz, 4H), 1.37 (dd, J=3.5, 6.3 Hz, 24H); ³¹P NMR (162 MHz, CDCl₃) δ=10.17.

Preparation of (7): To a mixture of 5 (6.84 g, 16.20 mmol) in THF (20 mL) was added LiBr (937.67 mg, 10.80 mmol) until dissolved, followed by DIEA (1.40 g, 10.80 mmol, 1.88 mL) under argon at 15° C. The mixture was stirred at 15° C. for 15 min. 6 (4 g, 10.80 mmol) were added. The mixture was stirred at 15° C. for 3 h. Upon completion as monitored by LCMS, the reaction mixture was quenched by addition of H₂O (40 mL) and extracted with EA (40 mL*3). The combined organic layers were washed with brine (100 mL), dried over Na₂SO₄, filtered and concentrated under reduced pressure to give a residue. The residue was purified by flash reverse-phase chromatography (120 g C-18 Column, Eluent of 0˜60% ACN/H₂O gradient @ 80 mL/min) to give 7 (5.7 g, 61.95% yield) as a colorless oil. ESI-LCMS: 611.2 [M+H]⁺; ¹H NMR (400 MHz, CDCl₃); δ=9.26 (s, 1H), 7.50 (d, J=8.1 Hz, 1H), 7.01 (s, 2H), 5.95 (d, J=2.7 Hz, 1H), 5.80 (dd, J=2.1, 8.2 Hz, 1H), 4.89-4.72 (m, 2H), 4.66 (d, J=7.2 Hz, 1H), 4.09-4.04 (m, 1H), 3.77 (dd, J=2.7, 4.9 Hz, 1H), 3.62 (d, J=3.1 Hz, 1H), 3.58 (d, J=3.1 Hz, 1H), 3.52 (s, 3H), 1.36 (td, J=1.7, 6.1 Hz, 12H), 0.92 (s, 9H), 0.12 (s, 6H); ³¹P NMR (162 MHz, CDCl₃) δ=9.02

Preparation of (8): To a mixture of 7 (5.4 g, 8.84 mmol) in THF (80 mL) was added Pd/C (5.4 g, 10% purity) under N₂. The suspension was degassed under vacuum and purged with H₂ several times. The mixture was stirred under H₂ (15 psi) at 20° C. for 1 hr. Upon completion as monitored by LCMS, the reaction mixture was filtered, and the filtrate was concentrated to give 8 (5.12 g, 94.5% yield) as a white solid. ESI-LCMS: 613.3 [M+H]⁺; ¹H NMR (400 MHz, CD₃CN) δ=9.31 (s, 1H), 7.37 (d, J=8.0 Hz, 1H), 5.80-5.69 (m, 2H), 4.87-4.75 (m, 2H), 4.11-4.00 (m, 1H), 3.93-3.85 (m, 1H), 3.80-3.74 (m, 1H), 3.66-3.60 (m, 1H), 3.57-3.52 (m, 1H), 3.49 (s, 3H), 3.46-3.38 (m, 1H), 2.35-2.24 (m, 1H), 2.16-2.03 (m, 1H), 1.89-1.80 (m, 1H), 1.37-1.34 (m, 12H), 0.90 (s, 9H), 0.09 (s, 6H); ³¹P NMR (162 MHz, CD₃CN) δ=9.41.

Preparation of (9): To a solution of 8 (4.4 g, 7.18 mmol) in THF (7.2 mL) was added TBAF (1 M, 7.18 mL), and the mixture was stirred at 20° C. for 1 hr. Upon completion as monitored by LCMS, the reaction mixture was diluted with H₂O (50 mL) and extracted with EA (50 mL*4). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜5%, MeOH/DCM gradient @ 40 mL/min) to give 9 (3.2 g, 88.50% yield) as a white solid. ESI-LCMS: 499.2 [M+H]⁺¹; ¹H NMR (400 MHz, CD₃CN) δ=9.21 (s, 1H), 7.36 (d, J=8.3 Hz, 1H), 5.81-5.72 (m, 2H), 4.88-4.74 (m, 2H), 3.99-3.87 (m, 2H), 3.84 (dd, J=1.9, 5.4 Hz, 1H), 3.66-3.47 (m, 7H), 2.98 (s, 1H), 2.44-2.15 (m, 2H), 1.36 (d, J=6.0 Hz, 12H); ³¹P NMR (162 MHz, CD₃CN) δ=9.48.

Preparation of (Example 2 monomer): To a mixture of 9 (3.4 g, 6.82 mmol, 1 eq) and 4A MS (3.4 g) in MeCN (50 mL) was added P1 (2.67 g, 8.87 mmol, 2.82 mL, 1.3 eq) at 0° C., followed by addition of 1H-imidazole-4,5-dicarbonitrile (886.05 mg, 7.50 mmol) at 0° C. The mixture was stirred at 20° C. for 2 h. Upon completion as monitored by LCMS, the reaction mixture was quenched by addition of saturated aq. NaHCO₃ (50 mL) and diluted with DCM (100 mL). The organic layer was washed with saturated aq. NaHCO₃ (50 mL*2), dried over Na₂SO₄, filtered and concentrated under reduced pressure to give a residue. The residue was purified by prep-HPLC: column: YMC-Triart Prep C18 250*50 mm*10 um; mobile phase: [water (10 mM NH₄HCO₃)-ACN]; B %: 15% to give a impure product. The impure product was further purified by a flash silica gel column (0% to 5% i-PrOH in DCM with 0.5% TEA) to give Example 2 monomer (2.1 g, 43.18% yield) as a white solid. ESI-LCMS: 721.2 [M+Na]⁺; H NMR (400 MHz, CD₃CN) δ=9.29 (s, 1H), 7.45 (d, J=8.1 Hz, 1H), 5.81 (d, J=4.2 Hz, 1H), 5.65 (d, J=8.1 Hz, 1H), 4.79-4.67 (m, 2H), 4.26-4.05 (m, 2H), 4.00-3.94 (m, 1H), 3.89-3.63 (m, 6H), 3.53-3.33 (m, 5H), 2.77-2.61 (m, 2H), 2.31-2.21 (m, 1H), 2.16-2.07 (m, 1H), 1.33-1.28 (m, 12H), 1.22-1.16 (m, 1H), 1.22-1.16 (m, 11H); ³¹P NMR (162 MHz, CD₃CN) δ=149.89, 149.78, 10.07, 10.02.

### Example 3. Synthesis of 5′ End Cap Monomer

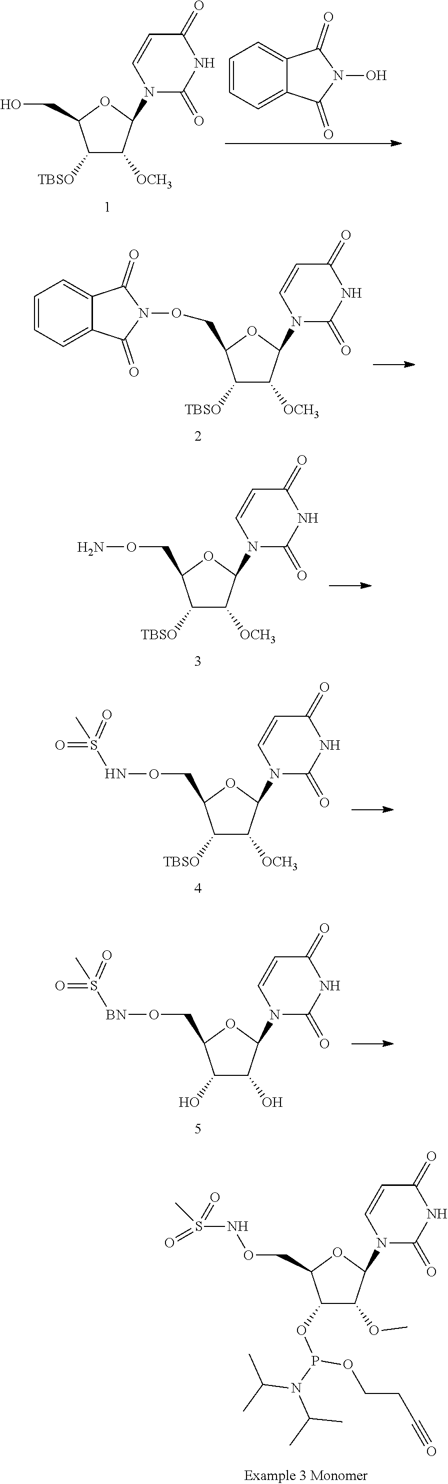

### Example 3 Monomer Synthesis Scheme

Preparation of (2): To a solution of 1 (5 g, 13.42 mmol) in DMF (50 mL) were added PPh₃ (4.58 g, 17.45 mmol) and 2-hydroxyisoindoline-1,3-dione (2.85 g, 17.45 mmol), followed by a solution of DIAD (4.07 g, 20.13 mmol, 3.91 mL) in DMF (10 mL) dropwise at 15° C. The resulting solution was stirred at 15° C. for 18 hr. The reaction mixture was then diluted with DCM (50 mL), washed with H₂O (60 mL*3) and brine (30 mL), dried over Na₂SO₄, filtered and evaporated to give a residue. The residue was then triturated with EtOH (55 mL) for 30 min, and the collected white powder was washed with EtOH (10 mL*2) and dried to give 2 (12.2 g, 85.16% yield) as a white powder (the reaction was set up in two batches and combined) ESI-LCMS: 518.1 [M+H]⁺.

Preparation of (3): 2 (6 g, 11.59 mmol) was suspended in MeOH (50 mL), and then NH₂NH₂·H₂O (3.48 g, 34.74 mmol, 3.38 mL, 50% purity) was added dropwise at 20° C. The reaction mixture was stirred at 20° C. for 4 hr. Upon completion, the reaction mixture was diluted with EA (20 mL) and washed with NaHCO₃ (10 mL*2) and brine (10 mL). The combined organic layers were then dried over Na₂SO₄, filtered and evaporated to give 3 (8.3 g, 92.5% yield) as a white powder. (The reaction was set up in two batches and combined). ESI-LCMS: 388.0 [M+H]⁺; ¹H NMR (400 MHz, DMSO-d₆) δ=11.39 (br s, 1H), 7.72 (d, J=8.1 Hz, 1H), 6.24-6.09 (m, 2H), 5.80 (d, J=4.9 Hz, 1H), 5.67 (d, J=8.1 Hz, 1H), 4.26 (t, J=4.9 Hz, 1H), 4.03-3.89 (m, 1H), 3.87-3.66 (m, 3H), 3.33 (s, 3H), 0.88 (s, 9H), 0.09 (d, J=1.3 Hz, 6H)

Preparation of (4): To a solution of 3 (7 g, 18.06 mmol) and Py (1.43 g, 18.06 mmol, 1.46 mL) in DCM (130 mL) was added a solution of MsCl (2.48 g, 21.68 mmol, 1.68 mL) in DCM (50 mL) dropwise at −78° C. under N₂. The reaction mixture was allowed to warm to 15° C. in 30 min and stirred at 15° C. for 3 h. The reaction mixture was quenched by addition of ice-water (70 mL) at 0° C., and then extracted with DCM (50 mL*3). The combined organic layers were washed with saturated aq. NaHCO₃ (50 mL) and brine (30 mL), dried over Na₂SO₄, filtered and concentrated under reduced pressure to give a residue. The residue was purified by flash silica gel chromatography (ISCOR; 30 g SepaFlash® Silica Flash Column, Eluent of 0˜20% i-PrOH/DCM gradient @ 30 mL/min to give 4 (6.9 g, 77.94% yield) as a white solid. ESI-LCMS: 466.1 [M+H]⁺; ¹H NMR (400 MHz, DMSO-d₆) δ=11.41 (br s, 1H), 10.15 (s, 1H), 7.69 (d, J=8.1 Hz, 1H), 5.80 (d, J=4.4 Hz, 1H), 5.65 (d, J=8.1 Hz, 1H), 4.24 (t, J=5.2 Hz, 1H), 4.16-3.98 (m, 3H), 3.87 (t, J=4.8 Hz, 1H), 3.00 (s, 3H), 2.07 (s, 3H), 0.88 (s, 9H), 0.10 (d, J=1.5 Hz, 6H)

Preparation of (5): To a solution of 4 (6.9 g, 14.82 mmol) in THF (70 mL) was added TBAF (1 M, 16.30 mL) at 15° C. The reaction mixture was stirred at 15° C. for 18 hr, and then evaporated to give a residue. The residue was purified by flash silica gel chromatography (ISCO®; 24 g SepaFlash® Silica Flash Column, Eluent of 0˜9% MeOH/Ethyl acetate gradient @ 30 mL/min) to give 5 (1.8 g, 50.8% yield) as a white solid. ESI-LCMS: 352.0 [M+H]⁺; ¹H NMR (400 MHz, DMSO-d₆) δ=11.40 (s, 1H), 10.13 (s, 1H), 7.66 (d, J=8.1 Hz, 1H), 5.83 (d, J=4.9 Hz, 1H), 5.65 (dd, J=1.8, 8.1 Hz, 1H), 5.36 (d, J=6.2 Hz, 1H), 4.13-4.00 (m, 4H), 3.82 (t, J=5.1 Hz, 1H), 3.36 (s, 3H), 3.00 (s, 3H)

Preparation of (Example 3 monomer): To a mixture of 5 (3 g, 8.54 mmol) and DIEA (2.21 g, 17.08 mmol, 2.97 mL) in ACN (90 mL) was added CEPCl (3.03 g, 12.81 mmol) dropwise at 15° C. The reaction mixture was stirred at 15° C. for 5 h. Upon completion, the reaction mixture was diluted with EA (40 mL) and quenched with 5% NaHCO₃ (20 mL). The organic layer was washed with brine (30 mL), dried over Na₂SO₄, filtered and evaporated to give a residue. The residue was purified by flash silica gel chromatography (ISCO®; 12 g SepaFlash® Silica Flash Column, Eluent of 0˜15% i-PrOH/(DCM with 2% TEA) gradient @ 20 mL/min) to Example 3 monomer (2.1 g, 43.93% yield) as a white solid. ESI-LCMS: 552.3 [M+H]⁺; ¹H NMR (400 MHz, CD₃CN) δ=8.78 (br s, 1H), 7.57 (dd, J=4.6, 8.2 Hz, 1H), 5.97-5.80 (m, 1H), 5.67 (d, J=8.3 Hz, 1H), 4.46-4.11 (m, 4H), 3.95-3.58 (m, 5H), 3.44 (d, J=16.3 Hz, 3H), 3.02 (d, J=7.5 Hz, 3H), 2.73-2.59 (m, 2H), 1.23-1.15 (m, 12H); ³¹P NMR (162 MHz, CD₃CN) δ=150.30, 150.10

### Example 4: Synthesis of 5′ End Cap Monomer

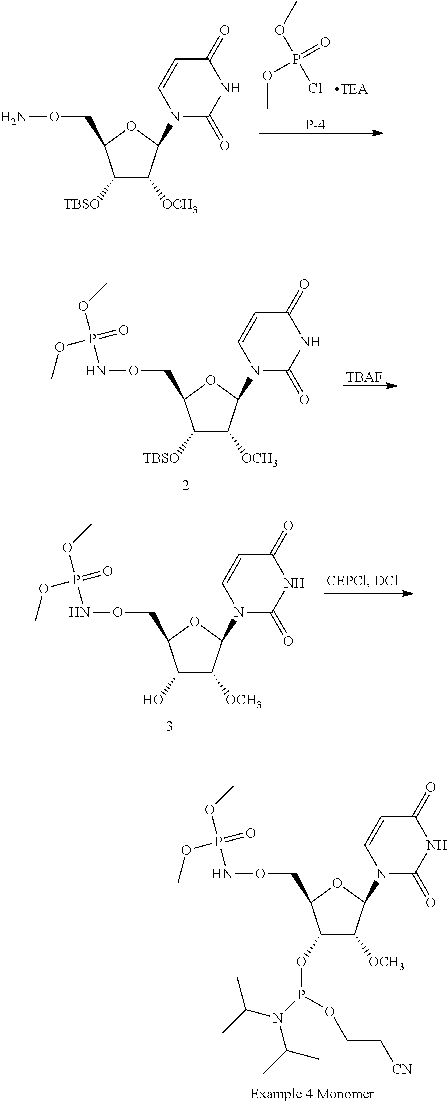

### Example 4 Monomer Synthesis Scheme

Preparation of (2): To the solution of 1 (5 g, 12.90 mmol) and TEA (1.57 g, 15.48 mmol, 2.16 mL) in DCM (50 mL) was added P-4 (2.24 g, 15.48 mmol, 1.67 mL) in DCM (10 mL) dropwise at 15° C. under N₂. The reaction mixture was stirred at 15° C. for 3 h. Upon completion as monitored by LCMS and TLC (PE:EtOAc=0:1), the reaction mixture was concentrated to dryness, diluted with H₂O (20 mL), and extracted with EA (50 mL*3). The combined organic layers were washed with brine (30 mL*3), dried over anhydrous Na₂SO₄, filtered, and the filtrate was concentrated under reduced pressure to give a residue. The residue was purified by flash silica gel chromatography (ISCO®; 40 g SepaFlash® Silica Flash Column, Eluent of 0˜95% Ethyl acetate/Petroleum ether gradient @ 60 mL/min) to give 2 (5.3 g, 71.3% yield) as a white solid. ESI-LCMS: 496.1 [M+H]⁺: H NMR (400 MHz, CDCl₃) δ=0.10 (d, J=4.02 Hz, 6H) 0.91 (s, 9H) 3.42-3.54 (m, 3H) 3.65-3.70 (m, 1H) 3.76-3.89 (m, 6H) 4.00 (dd, J=10.92, 2.89 Hz, 1H) 4.08-4.13 (m, 1H) 4.15-4.23 (m, 2H) 5.73 (dd, J=8.28, 2.01 Hz, 1H) 5.84 (d, J=2.76 Hz, 1H) 6.86 (d, J=15.81 Hz, 1H) 7.72 (d, J=8.03 Hz, 1H) 9.10 (s, 1H); ³¹P NMR (162 MHz, CD₃CN) δ=9.65

Preparation of (3): To a solution of 2 (8.3 g, 16.75 mmol) in THF (50 mL) were added TBAF (1 M, 16.75 mL) and CH₃COOH (1.01 g, 16.75 mmol, 957.95 uL). The mixture was stirred at 20° C. for 12 hr. Upon completion as monitored by LCMS, the reaction mixture was concentrated under reduced pressure. The residue was purified by column chromatography (SiO₂, PE:EA=0˜100%; MeOH/EA=0˜10%) to give 3 (5 g, 77.51% yield) as a white solid. ESI-LCMS: 382.1 [M+H]⁺; ¹H NMR (400 MHz, CDCl₃) δ=3.35 (s, 3H) 3.65 (br d, J=2.76 Hz, 3H) 3.68 (d, J=2.76 Hz, 3H) 3.77 (t, J=5.08 Hz, 1H) 3.84-4.10 (m, 4H) 5.33 (br d, J=5.52 Hz, 1H) 5.62 (d, J=7.77 Hz, 1H) 5.83 (d, J=4.94 Hz, 1H) 7.69 (d, J=7.71 Hz, 1H) 9.08 (d, J=16.81 Hz, 1H) 11.39 (br s, 1H); ³¹P NMR (162 MHz, CD₃CN) δ=15.41

Preparation of (Example 4 monomer): To a solution of 3 (2 g, 5.25 mmol) and DIPEA (2.03 g, 15.74 mmol, 2.74 mL, 3 eq) in MeCN (21 mL) and pyridine (7 mL) was added CEOP[N(iPr)₂]₂/CEP[N(iPr)₂]₂/CEP/CEPCl (1.86 g, 7.87 mmol) dropwise at 20° C., and the mixture was stirred at 20° C. for 3 hr. Upon completion as monitored by LCMS, the reaction mixture was diluted with water (20 mL) and extracted with EA (50 mL). The combined organic layers were washed with brine (30 mL), dried over anhydrous Na₂SO₄, filtered, and the filtrate was concentrated under reduced pressure to give a residue. The residue was purified by flash silica gel chromatography (ISCO®; 25 g SepaFlash® Silica Flash Column, Eluent of 0˜45% (Ethyl acetate:EtOH=4:1)/Petroleum ether gradient) to give Example 4 monomer (1.2 g, 38.2% yield) as a white solid. ESI-LCMS: 604.1 [M+H]⁺. ¹H NMR (400 MHz, CD₃CN) δ=1.12-1.24 (m, 12H) 2.61-2.77 (m, 2H) 3.43 (d, J=17.64 Hz, 3H) 3.59-3.69 (m, 2H) 3.71-3.78 (m, 6H) 3.79-4.14 (m, 5H) 4.16-4.28 (m, 1H) 4.29-4.42 (m, 1H) 5.59-5.72 (m, 1H) 5.89 (t, J=4.53 Hz, 1H) 7.48 (br d, J=12.76 Hz, 1H) 7.62-7.74 (m, 1H) 9.26 (br s, 1H); ³¹P NMR (162 MHz, CD₃CN) δ=150.57, 149.96, 9.87

### Example 5: Synthesis of 5′ End Cap Monomer

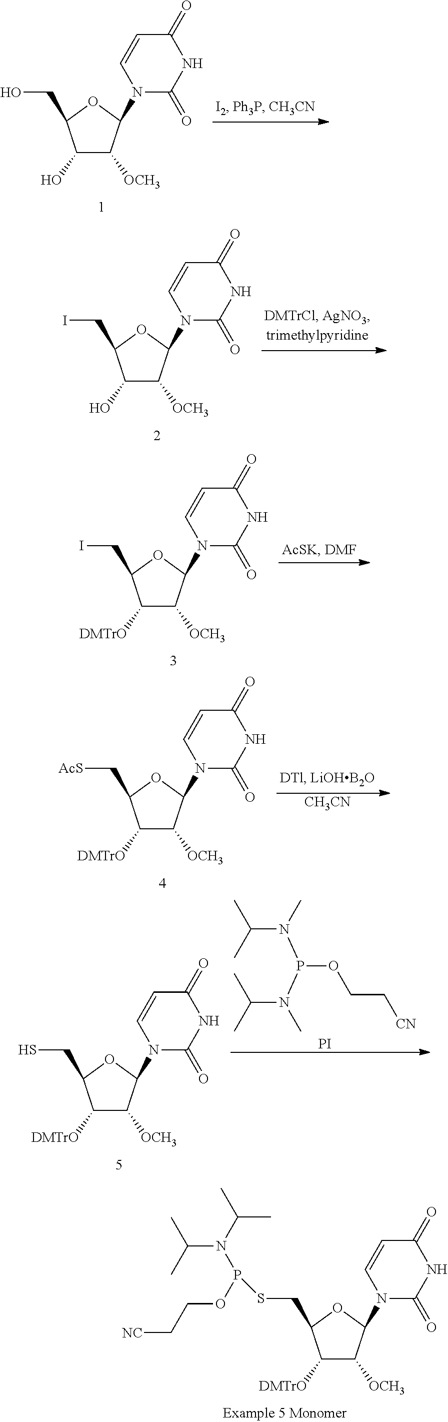

### Example 5 Monomer Synthesis Scheme

Preparation of (2): To a solution of 1 (30 g, 101.07 mmol, 87% purity) in CH₃CN (1.2 L) and Py (60 mL) were added I₂ (33.35 g, 131.40 mmol, 26.47 mL) and PPh₃ (37.11 g, 141.50 mmol) in one portion at 10° C. The reaction was stirred at 25° C. for 48 h. Upon completion, the mixture was diluted with saturated aq. Na₂S₂O₃ (300 mL) and saturated aq. NaHCO₃ (300 mL), concentrated to remove CH₃CN, and extracted with EtOAc (300 mL*3). The combined organic layers were washed with brine (300 mL), dried over Na₂SO₄, filtered and concentrated under reduced pressure to give a residue. The residue was purified by flash silica gel chromatography (ISCO®; 330 g SepaFlash® Silica Flash Column, Eluent of 0-60% Methanol/Dichloromethane gradient @ 100 mL/min) to give 2 (28.2 g, 72% yield) as a brown solid. ESI-LCMS: 369.1 [M+H]⁺: H NMR (400 MHz, DMSO-d₆) δ=11.43 (s, 1H), 7.68 (d, J=8.1 Hz, 1H), 5.86 (d, J=5.5 Hz, 1H), 5.69 (d, J=8.1 Hz, 1H), 5.46 (d, J=6.0 Hz, 1H), 4.08-3.96 (m, 2H), 3.90-3.81 (m, 1H), 3.60-3.51 (m, 1H), 3.40 (dd, J=6.9, 10.6 Hz, 1H), 3.34 (s, 3H).

Preparation of (3): To the solution of 2 (12 g, 32.6 mmol) in DCM (150 mL) were added AgNO₃ (11.07 g, 65.20 mmol), 2,4,6-trimethylpyridine (11.85 g, 97.79 mmol, 12.92 mL), and DMTCl (22.09 g, 65.20 mmol) at 10° C., and the reaction mixture was stirred at 10° C. for 16 hr. Upon completion, the mixture was filtered and the filtrate was concentrated under reduced pressure. The residue was purified by flash silica gel chromatography (ISCO®; 120 g SepaFlash® Silica Flash Column, Eluent of 0˜50% Ethyl acetate/Petroleum ether gradient @ 60 mL/min) to give 3 (17 g, 70.78% yield) as a yellow solid. ESI-LCMS: 693.1 [M+Na]⁺¹; H NMR (400 MHz, DMSO-d₆) δ=11.46 (s, 1H), 7.60 (d, J=8.4 Hz, 1H), 7.49 (d, J=7.2 Hz, 2H), 7.40-7.30 (m, 6H), 7.29-7.23 (m, 1H), 6.93 (d, J=8.8 Hz, 4H), 5.97 (d, J=6.0 Hz, 1H), 5.69 (d, J=8.0 Hz, 1H), 4.05-4.02 (m, 1H), 3.75 (d, J=1.2 Hz, 6H), 3.57 (t, J=5.6 Hz, 1H), 3.27 (s, 4H), 3.06 (t, J=10.4 Hz, 1H), 2.98-2.89 (m, 1H).

Preparation of (4): To a solution of 3 (17 g, 25.35 mmol) in DMF (200 mL) was added AcSK (11.58 g, 101.42 mmol) at 25° C., and the reaction was stirred at 60° C. for 2 hr. The mixture was diluted with H₂O (600 mL) and extracted with EtOAc (300 mL*4). The combined organic layers were washed with brine (300 mL), dried over Na₂SO₄, filtered, and concentrated under reduced pressure to give 4 (15.6 g, crude) as a brown solid, which was used directly without further purification. ESI-LCMS: 641.3 [M+H]⁺.

Preparation of (5): To a solution of 4 (15.6 g, 25.21 mmol) in CH₃CN (200 mL) were added DTT (11.67 g, 75.64 mmol, 11.22 mL) and LiOH·H₂O (1.06 g, 25.21 mmol) at 10° C. under Ar. The reaction was stirred at 10° C. for 1 hr. The mixture was concentrated under reduced pressure to remove CH₃CN, and the residue was diluted with H₂O (400 mL) and extracted with EtOAc (200 mL*3). The combined organic layers were washed with brine (300 mL), dried over Na₂SO₄, filtered and concentrated under reduced pressure to give a residue. The residue was purified by flash silica gel chromatography (ISCO®; 220 g SepaFlash® Silica Flash Column, Eluent of 0˜60% Ethyl acetate/Petroleum ether gradient @ 100 mL/min) to give 5 (8.6 g, 56.78% yield) as a white solid. ESI-LCMS: 599.3 [M+Na]⁺; ¹H NMR (400 MHz, DMSO-d₆) δ=8.79 (s, 1H), 7.61 (d, J=8.0 Hz, 1H), 7.56-7.46 (m, 2H), 7.45-7.37 (m, 4H), 7.36-7.27 (m, 3H), 6.85 (dd, J=2.8, 8.8 Hz, 4H), 5.85 (d, J=1.3 Hz, 1H), 5.68 (dd, J=2.0, 8.2 Hz, 1H), 4.33-4.29 (m, 1H), 3.91 (dd, J=4.8, 8.2 Hz, 1H), 3.81 (d, J=1.6 Hz, 6H), 3.33 (s, 3H), 2.85-2.80 (m, 1H), 2.67-2.55 (m, 2H), 1.11 (t, J=8.8 Hz, 1H).

Preparation of (Example 5 monomer): To a solution of 5 (6 g, 10.40 mmol) in DCM (120 mL) were added P1 (4.08 g, 13.53 mmol, 4.30 mL) and DCI (1.35 g, 11.45 mmol) in one portion at 10° C. under Ar. The reaction was stirred at 10° C. for 2 hr. The reaction mixture was diluted with saturated aq. NaHCO₃ (50 mL) and extracted with DCM (20 mL*3). The combined organic layers were washed with brine (30 mL), dried over Na₂SO₄, filtered and concentrated under reduced pressure to give a residue. The residue was purified by prep-HPLC (column: YMC-Triart Prep C18 250*50 mm*10 um; mobile phase: [water (10 mM NH₄HCO₃)-ACN]; B %: 35%-81%, 20 min) to give Example 5 monomer (3.54 g, 43.36% yield) as a yellow solid. ESI-LCMS: 776.4 [M+H]⁺; ¹H NMR (400 MHz, DMSO-d₆) δ=7.65-7.38 (m, 7H), 7.37-7.22 (m, 3H), 6.90 (d, J=8.4 Hz, 4H), 5.92 (s, 1H), 5.66 (t, J=8.2 Hz, 1H), 4.13 (d, J=4.0 Hz, 1H), 4.00-3.88 (m, 1H), 3.87-3.59 (m, 10H), 3.33 (d, J=5.8 Hz, 3H), 3.12-2.94 (m, 1H), 2.78-2.60 (m, 3H), 2.55-2.48 (m, 1H), 1.36-0.98 (m, 12H); ³¹P NMR (162 MHz, DMSO-d₆) δ=162.69.

### Example 6: Synthesis of 5′ End Cap Monomer

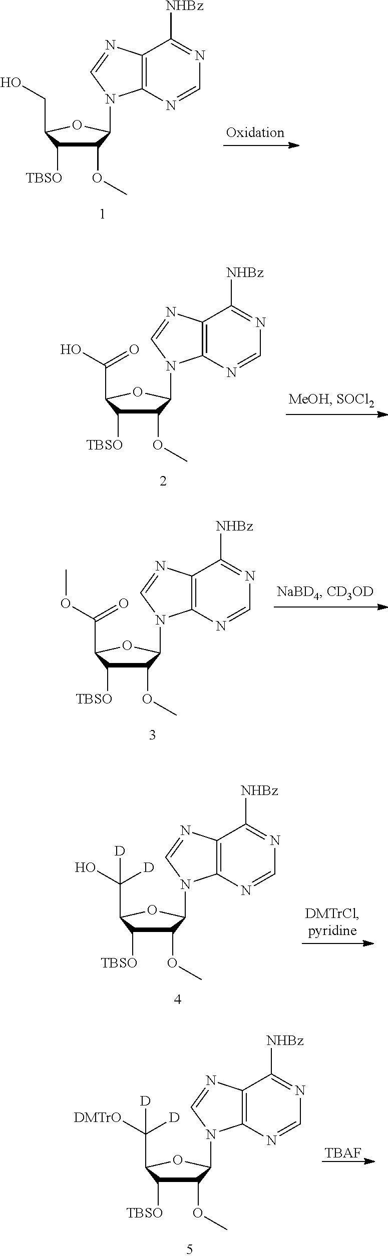

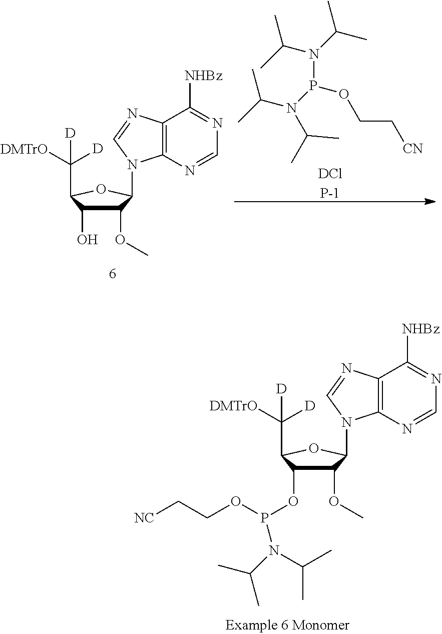

### Example 6 Monomer Synthesis Scheme

Preparation of (2): To a solution of 1 (22.6 g, 45.23 mmol) in DCM (500 mL) and H₂O (125 mL) were added TEMPO (6.40 g, 40.71 mmol) and DIB (29.14 g, 90.47 mmol) at 0° C. The mixture was stirred at 20° C. for 20 h. Upon completion as monitored by LCMS, saturated aq. NaHCO₃ was added to the mixture to adjust pH >8. The mixture was diluted with H₂O (200 mL) and washed with DCM (100 mL*3). The aqueous layer was collected, adjusted to pH <5 by HCl (4M), and extracted with DCM (200 mL*3). The combined organic layers were washed with brine (300 mL), dried over Na₂SO₄, filtered, and concentrated under reduced pressure to give 2 (17.5 g, 68.55% yield) as a yellow solid. ESI-LCMS: 514.2 [M+H]⁺; 1H NMR (400 MHz, DMSO-d₆) δ=11.27 (s, 1H), 8.86 (s, 1H), 8.78 (s, 1H), 8.06 (d, J=7.5 Hz, 2H), 7.68-7.62 (m, 1H), 7.59-7.52 (m, 2H), 6.28 (d, J=6.8 Hz, 1H), 4.82-4.76 (m, 1H), 4.54 (dd, J=4.1, 6.7 Hz, 1H), 4.48 (d, J=1.8 Hz, 1H), 3.32 (s, 3H), 0.94 (s, 9H), 0.18 (d, J=4.8 Hz, 6H).

Preparation of (3): To a solution of 2 (9.3 g, 18.11 mmol) in MeOH (20 mL) was added SOCl₂ (3.23 g, 27.16 mmol, 1.97 mL) dropwise at 0° C. The mixture was stirred at 20° C. for 0.5 hr. Upon completion as monitored by LCMS, the reaction mixture was quenched by addition of saturated aq. NaHCO₃ (80 mL) and concentrated under reduced pressure to remove MeOH. The aqueous layer was extracted with DCM (80 mL*3). The combined organic layers were washed with brine (200 mL), dried over Na₂SO₄, filtered and concentrated under reduced pressure to give a residue. The residue was purified by flash silica gel chromatography (ISCOR; 120 g SepaFlash® Silica Flash Column, Eluent of 0˜5%, MeOH/DCM gradient @ 85 mL/min) to give 3 (5.8 g, 60% yield) as a yellow solid. ESI-LCMS: 528.3 [M+H]⁺; ¹H NMR (400 MHz, DMSO-d₆) δ=11.28 (s, 1H), 8.79 (d, J=7.3 Hz, 2H), 8.06 (d, J=7.5 Hz, 2H), 7.68-7.62 (m, 1H), 7.60-7.53 (m, 2H), 6.28 (d, J=6.6 Hz, 1H), 4.87 (dd, J=2.4, 4.0 Hz, 1H), 4.61 (dd, J=4.3, 6.5 Hz, 1H), 4.57 (d, J=2.2 Hz, 1H), 3.75 (s, 3H), 3.32 (s, 3H), 0.94 (s, 9H), 0.17 (d, J=2.2 Hz, 6H).

Preparation of (4): To a mixture of 3 (5.7 g, 10.80 mmol) in CD₃OD (120 mL) was added NaBD₄ (1.63 g, 43.21 mmol) in portions at 0° C., and the mixture was stirred at 20° C. for 1 hr. Upon completion as monitored by LCMS, the reaction mixture was neutralized by AcOH (˜10 mL) and concentrated under reduced pressure to give a residue. The residue was purified by flash silica gel chromatography (ISCO®; 40 g SepaFlash® Silica Flash Column, Eluent of 0˜5%, MeOH/DCM gradient @ 40 mL/min) to give 4 (4.15 g, 7.61 mmol, 70.45% yield) as a yellow solid. ESI-LCMS: 502.2 [M+H]⁺; ¹H NMR (400 MHz, DMSO-d₆) δ=11.23 (s, 1H), 8.76 (s, 2H), 8.04 (d, J=7.3 Hz, 2H), 7.69-7.62 (m, 1H), 7.60-7.52 (m, 2H), 6.14 (d, J=6.0 Hz, 1H), 5.18 (s, 1H), 4.60-4.51 (m, 2H), 3.98 (d, J=3.0 Hz, 1H), 3.32 (s, 3H), 0.92 (s, 9H), 0.13 (d, J=1.5 Hz, 6H).

Preparation of (5): To a solution of 4 (4.85 g, 9.67 mmol) in pyridine (50 mL) was added DMTrCl (5.90 g, 17.40 mmol) at 25° C. and the mixture was stirred for 2 hr. Upon completion as monitored by LCMS, the reaction mixture was concentrated under reduced pressure to remove pyridine. The residue was diluted with EtOAc (150 mL) and washed with H₂O (50 mL*3), dried over Na₂SO₄, filtered and concentrated under reduced pressure to give a residue. The residue was purified by flash silica gel chromatography (ISCO®; 80 g SepaFlash® Silica Flash Column, Eluent of 0˜70%, EA/PE gradient @ 60 mL/min) to give 5 (6.6 g, 84.06% yield) as a yellow solid. ESI-LCMS: 804.3 [M+H], ¹H NMR (400 MHz, DMSO-d₆) δ=11.22 (s, 1H), 8.68 (d, J=11.0 Hz, 2H), 8.03 (d, J=7.3 Hz, 2H), 7.68-7.60 (m, 1H), 7.58-7.49 (m, 2H), 7.37-7.30 (m, 2H), 7.27-7.16 (m, 7H), 6.88-6.79 (m, 4H), 6.17 (d, J=4.2 Hz, 1H), 4.72 (t, J=5.0 Hz, 1H), 4.60 (t, J=4.5 Hz, 1H), 4.03-3.98 (m, 1H), 3.71 (s, 6H), 0.83 (s, 9H), 0.12-0.03 (m, 6H).

Preparation of (6): To a solution of 5 (6.6 g, 8.21 mmol) in THF (16 mL) was added TBAF (1 M, 8.21 mL), and the mixture was stirred at 20° C. for 2 hr. Upon completion as monitored by LCMS, the reaction mixture was diluted with EA (150 mL) and washed with H₂O (50 mL*3). The organic layer was washed with brine (150 mL), dried over Na₂SO₄, filtered and concentrated under reduced pressure to give a residue. The residue was purified by flash silica gel chromatography (ISCO®; 80 g SepaFlash® Silica Flash Column, Eluent of 10-100%, EA/PE gradient @ 30 mL/min) to give 6 (5.4 g, 94.4% yield) as a yellow solid. ESI-LCMS: 690.3 [M+H]⁺; ¹H NMR (400 MHz, DMSO-d₆) δ=11.24 (s, 1H), 8.69 (s, 1H), 8.62 (s, 1H), 8.05 (d, J=7.3 Hz, 2H), 7.69-7.62 (m, 1H), 7.60-7.52 (m, 2H), 7.40-7.33 (m, 2H), 7.30-7.18 (m, 7H), 6.84 (dd, J=5.9, 8.9 Hz, 4H), 6.19 (d, J=4.8 Hz, 1H), 5.36 (d, J=6.0 Hz, 1H), 4.59-4.52 (m, 1H), 4.48 (q, J=5.1 Hz, 1H), 4.11 (d, J=4.8 Hz, 1H), 3.72 (d, J=1.0 Hz, 6H), 3.40 (s, 3H).

Preparation of (Example 6 monomer): To a solution of 6 (8.0 g, 11.60 mmol) in MeCN (150 mL) was added P-1 (4.54 g, 15.08 mmol, 4.79 mL) at 0° C., followed by DCI (1.51 g, 12.76 mmol) in one portion. The mixture was warmed to 20° C. and stirred for 2 h. Upon completion as monitored by LCMS, the reaction mixture was quenched by addition of saturated aq. NaHCO₃ (50 mL) and diluted with DCM (250 mL). The organic layer was washed with saturated aq. NaHCO₃ (50 mL*2), dried over Na₂SO₄, filtered and concentrated under reduced pressure. The residue was purified by a flash silica gel column (0% to 60% EA in PE contain 0.5% TEA) to give Example 6 monomer (5.75 g, 55.37% yield, 99.4% purity) as a white solid. ESI-LCMS: 890.4 [M+H]⁺; ¹H NMR (400 MHz, CD₃CN) δ=9.55 (s, 1H), 8.63-8.51 (m, 1H), 8.34-8.24 (m, 1H), 7.98 (br d, J=7.5 Hz, 2H), 7.65-7.55 (m, 1H), 7.53-7.46 (m, 2H), 7.44-7.37 (m, 2H), 7.32-7.17 (m, 7H), 6.84-6.77 (m, 4H), 6.14 (d, J=4.3 Hz, 1H), 4.84-4.73 (m, 1H), 4.72-4.65 (m, 1H), 4.34-4.27 (m, 1H), 3.91-3.61 (m, 9H), 3.50-3.43 (m, 3H), 2.72-2.61 (m, 1H), 2.50 (t, J=6.0 Hz, 1H), 1.21-1.15 (m, 10H), 1.09 (d, J=6.8 Hz, 2H); ³¹P NMR (162 MHz, CD₃CN) δ=150.01, 149.65

### Example 7: Synthesis of 5′ End Cap Monomer

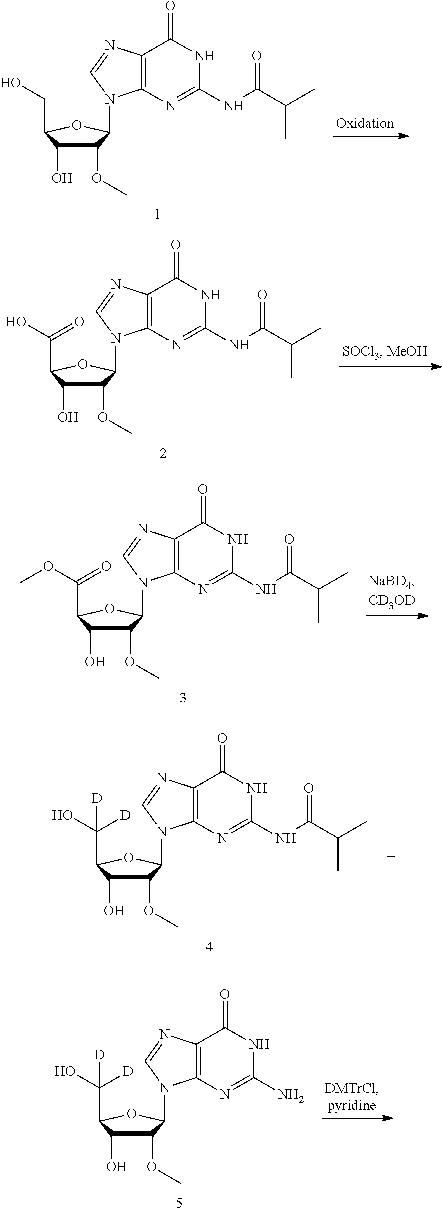

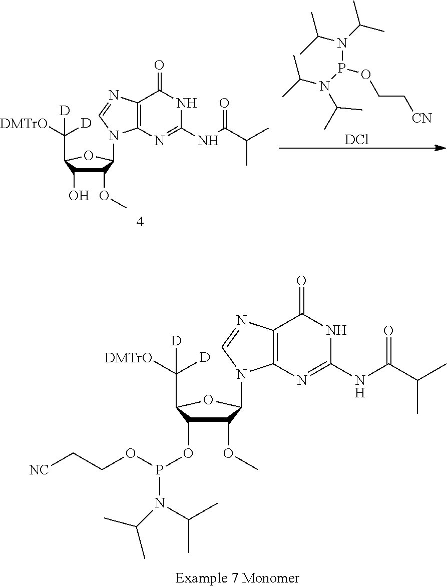

### Example 7 Monomer Synthesis Scheme

Preparation of (2): To a solution of 1 (10 g, 27.22 mmol) in CH₃CN (200 mL) and H₂O (50 mL) were added TEMPO (3.85 g, 24.50 mmol) and DIB (17.54 g, 54.44 mmol). The mixture was stirred at 25° C. for 12 h. Upon completion as monitored by LCMS, the reaction mixture was concentrated under reduced pressure to give a residue. The residue was triturated with EtOAc (600 mL) for 30 min. The resulting suspension was filtered and the collected solid was washed with EtOAc (300 mL*2) to give 2 (20.09 g, 91.5% yield) as a white solid. ESI-LCMS: 382.0 [M+H]⁺.

Preparation of (3): To a solution of 2 (6 g, 15.73 mmol) in MeOH (100 mL) was added SOCl₂ (2.81 g, 23.60 mmol, 1.71 mL) dropwise at 0° C. The mixture was stirred at 25° C. for 12 h. Upon completion as monitored by LCMS, the reaction mixture was quenched by addition of NaHCO₃ (4 g) and stirred at 25° C. for 30 min. The reaction mixture was filtered and the filtrate was concentrated under reduced pressure to give 3 (18.8 g, 95.6% yield) as a white solid. The crude product was used for the next step without further purification. (The reaction was set up in parallel 3 batches and combined). ESI-LCMS: 396.1 [M+H]⁺; ¹H NMR (400 MHz, DMSO-d₆) δ=12.26-11.57 (m, 2H), 8.42-8.06 (m, 1H), 6.14-5.68 (m, 2H), 4.56 (s, 2H), 4.33 (dd, J=4.0, 7.3 Hz, 1H), 3.77 (m, 3H), 3.30 (s, 3H), 2.81-2.69 (m, 1H), 1.11 (s, 6H)

Preparation of (4 & 5): To a mixture of 3 (10.1 g, 25.55 mmol) in CD₃OD (120 mL) was added NaBD₄ (3.29 g, 86.86 mmol, 3.4 eq) in portions at 0° C. The mixture was stirred at 25° C. for 1 h. Upon completion as monitored by LCMS, the reaction mixture was neutralized with AcOH (˜15 mL) and concentrated under reduced pressure to give a residue. The residue was purified by flash silica gel chromatography (ISCO®; 120 g SepaFlash® Silica Flash Column, Eluent of 0˜7.4%, MeOH/DCM gradient @ 80 mL/min) to give 4 (2.98 g, 6.88 mmol, 27% yield) as a yellow solid. ESI-LCMS: 370.1 [M+H]⁺ and 5 (10.9 g, crude) as a yellow solid. ESI-LCMS: 300.1 [M+H]⁺; ¹H NMR (400 MHz, CD₃OD) δ=7.85 (s, 1H), 5.87 (d, J=6.0 Hz, 1H), 4.46-4.39 (m, 1H), 4.34 (t, J=5.4 Hz, 1H), 4.08 (d, J=3.1 Hz, 1H), 3.49-3.38 (m, 4H)

Preparation of 6: To a solution of 4 (1.9 g, 4.58 mmol, 85.7% purity) in pyridine (19 mL) was added DMTrCl (2.02 g, 5.96 mmol). The mixture was stirred at 25° C. for 2 h under N₂. Upon completion as monitored by LCMS, the reaction mixture was quenched by MeOH (10 mL) and concentrated under reduce pressure to give a residue. The residue was diluted with H₂O (10 mL*3) and extracted with EA (20 mL*3). The combined organic layers were washed with brine (20 mL), dried over anhydrous Na₂SO₄, filtered and concentrated under reduce pressure to give a residue. The residue was purified by flash silica gel chromatography (ISCO®; 25 g SepaFlash® Silica Flash Column, Eluent of 0˜77%, PE: (EA with 10% EtOH): 1% TEA@ 35 mL/min) to give 6 (2.6 g, 81.71% yield, 96.71% purity) as a white foam. ESI-LCMS: 672.2 [M+H]⁺; ¹H NMR (400 MHz, CDCl₃) δ=12.02 (s, 1H), 7.96 (s, 1H), 7.83 (s, 1H), 7.51 (d, J=7.4 Hz, 2H), 7.37 (d, J=8.6 Hz, 4H), 7.25-7.17 (m, 2H), 6.80 (t, J=8.4 Hz, 4H), 5.88 (d, J=6.3 Hz, 1H), 4.69 (t, J=5.7 Hz, 1H), 4.64 (s, 1H), 4.54 (s, 1H), 4.19 (d, J=2.9 Hz, 1H), 3.77 (d, J=4.5 Hz, 6H), 3.60-3.38 (m, 3H), 2.81 (s, 1H), 1.81 (td, J=6.9, 13.7 Hz, 1H), 0.97 (d, J=6.8 Hz, 3H), 0.80 (d, J=6.9 Hz, 3H).

Preparation of Example 7 monomer: To a solution of 6 (8.4 g, 12.5 mmol) in MeCN (80 mL) was added P-1 (4.9 g, 16.26 mmol, 5.16 mL) at 0° C., followed by addition of DCI (1.624 g, 13.76 mmol) in one portion at 0° C. under Ar. The mixture was stirred at 25° C. for 2 h. Upon completion as monitored by LCMS, the reaction mixture was quenched with saturated aq. NaHCO₃ (20 mL) and extracted with DCM (50 mL*2). The combined organic layers were dried over anhydrous Na₂SO₄, filtered and concentrated under reduce pressure to give a residue. The residue was purified by flash silica gel chromatography (ISCO®; 40 g SepaFlash® Silica Flash Column, Eluent of 0˜52% PE:EA (10% EtOH): 5% TEA, @ 80 mL/min) to give Example 7 monomer (3.4 g, 72.1% yield) as a white foam. ESI-LCMS: 872.4 [M+H]⁺; ¹H NMR (400 MHz, CD₃CN) δ=12.46-11.07 (m, 1H), 9.29 (s, 1H), 7.84 (d, J=14.6 Hz, 1H), 7.42 (t, J=6.9 Hz, 2H), 7.34-7.17 (m, 7H), 6.85-6.77 (m, 4H), 5.95-5.77 (m, 1H), 4.56-4.40 (m, 2H), 4.24 (dd, J=4.0, 13.3 Hz, 1H), 3.72 (d, J=2.0 Hz, 7H), 3.66-3.53 (m, 3H), 3.42 (d, J=11.8 Hz, 3H), 2.69-2.61 (m, 1H), 2.60-2.42 (m, 2H), 1.16-1.00 (m, 18H); ³¹P NMR (162 MHz, CD₃CN) δ=149.975, 149.9.

### Example 8: Synthesis of 5′ End Cap Monomer

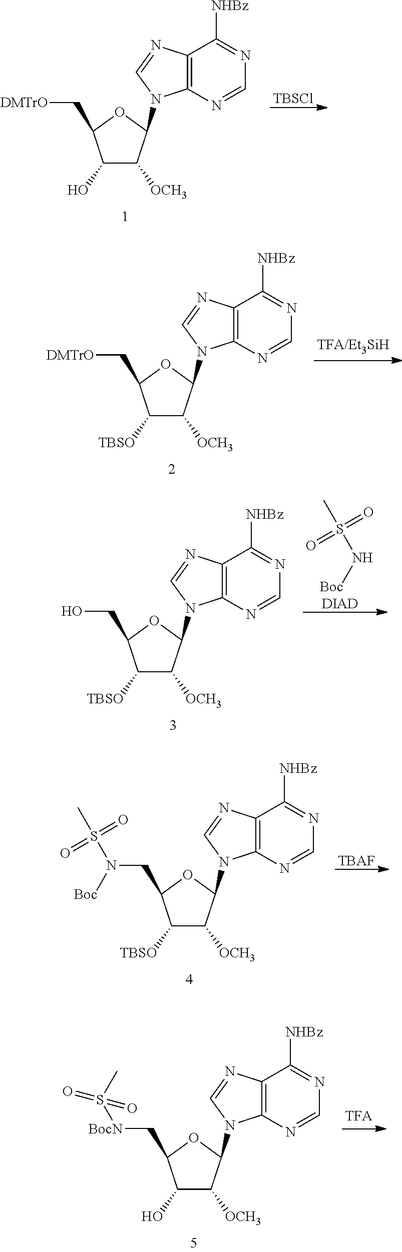

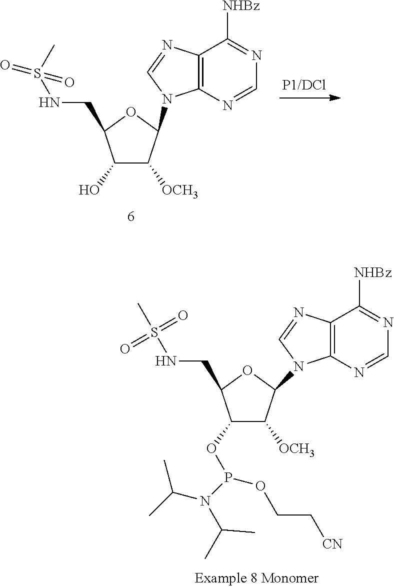

### Example 8 Monomer Synthesis Scheme

Preparation of (2): To a solution of 1 (40 g, 58.16 mmol) in DMF (60 mL) were added imidazole (11.88 g, 174.48 mmol), NaI (13.08 g, 87.24 mmol), and TBSCl (17.52 g, 116.32 mmol) at 20° C. in one portion. The reaction mixture was stirred at 20° C. for 12 h. Upon completion, the mixture was diluted with EA (200 mL). The organic layer was washed with brine/water (80 mL/80 mL*4), dried over Na₂SO₄, filtered and evaporated to give 2 (50.8 g, crude) as yellow solid. ESI-LCMS: 802.3 [M+H]⁺

Preparation of (3): To a solution of 2 (8.4 g, 10.47 mmol) in DCM (120 mL) were added Et₃SiH (3.06 g, 26.3 mmol, 4.2 mL) and TFA (1.29 g, 0.84 mL) dropwise at 0° C. The reaction mixture was stirred at 20° C. for 2 h. The reaction mixture was washed with saturated aq·NaHCO₃ (15 mL) and brine (80 mL). The organic layer was dried over Na₂SO₄, filtered and evaporated. The residue was purified by flash silica gel chromatography (ISCO®; 80 g SepaFlash® Silica Flash Column, Eluent of 0˜83% EA/PE gradient @ 80 mL/min) to give 3 (2.92 g, 55.8% yield) as a white solid. ESI-LCMS: 500.2 [M+H]⁺; ¹H NMR (400 MHz, CDCl₃) δ=8.79 (s, 1H), 8.14 (s, 1H), 8.02 (d, J=7.6 Hz, 2H), 7.64-7.58 (m, 1H), 7.56-7.49 (m, 2H), 5.98-5.93 (m, 1H), 4.63-4.56 (m, 2H), 4.23 (s, 1H), 3.98 (dd, J=1.5, 13.1 Hz, 1H), 3.75 (dd, J=1.5, 13.1 Hz, 1H), 3.28 (s, 3H), 2.06-1.99 (m, 1H), 1.00-0.90 (m, 9H), 0.15 (d, J=7.0 Hz, 6H).

Preparation of (4): 3 (6 g, 12.01 mmol) and tert-butyl N-methylsulfonylcarbamate (3.52 g, 18.01 mmol) were co-evaporated with toluene (50 mL), dissolved in dry THF (100 mL), and cooled to 0° C. PPh₃ (9.45 g, 36.03 mmol) was then added, followed by dropwise addition of DIAD (7.28 g, 36.03 mmol, 7.00 mL) in dry THF (30 mL). The reaction mixture was stirred at 20° C. for 18 h. Upon completion, the reaction mixture was then diluted with DCM (100 mL) and washed with water (70 mL) and brine (70 mL), dried over Na₂SO₄, filtered and evaporated to give a residue. The residue was purified by flash silica gel chromatography (ISCO®; 80 g SepaFlash® Silica Flash Column, Eluent of 0˜100% Ethyl acetate/Petroleum ether gradient @ 60 mL/min) followed by reverse-phase HPLC (0.1% NH₃·H₂O condition, eluent at 74%) to give 4 (2.88 g, 25% yield) as a white solid. ESI-LCMS: 677.1 [M+H]⁺; ¹H NMR (400 MHz, CDCl₃) δ=9.24 (s, 1H), 8.84 (s, 1H), 8.36 (s, 1H), 8.05 (br d, J=7.3 Hz, 2H), 7.66-7.42 (m, 4H), 6.16 (d, J=5.0 Hz, 1H), 4.52 (br t, J=4.5 Hz, 1H), 4.25-4.10 (m, 1H), 3.97 (br dd, J=8.0, 14.8 Hz, 1H), 3.48 (s, 3H), 3.27 (s, 3H), 1.54 (s, 9H), 0.95 (s, 9H), 0.14 (d, J=0.8 Hz, 6H).

Preparation of (5): To a solution of 4 (2.8 g, 4.14 mmol) in THF (20 mL) was added TBAF (4 M, 1.03 mL) and the mixture was stirred at 20° C. for 12 h. The reaction mixture was then evaporated. The residue was purified by flash silica gel chromatography (ISCO®; 12 g SepaFlash® Silica Flash Column, Eluent of 0˜6% MeOH/ethyl acetate gradient @ 20 mL/min) to give 5 (2.1 g, 83.92% yield) as a white solid. ESI-LCMS: 563.1 [M+H]⁺; ¹H NMR (400 MHz, CDCl₃) δ=8.85-8.77 (m, 1H), 8.38 (s, 1H), 8.11-7.99 (m, 2H), 7.64-7.50 (m, 4H), 6.19 (d, J=2.8 Hz, 1H), 4.36-4.33 (m, 1H), 4.29 (br d, J=4.3 Hz, 1H), 4.22-4.02 (m, 2H), 3.65-3.59 (m, 3H), 3.28 (s, 3H), 1.54 (s, 9H).

Preparation of (6): To a solution of 5 (2.1 g, 3.73 mmol) in DCM (20 mL) was added TFA (7.70 g, 67.53 mmol, 5 mL) at 0° C. The reaction mixture was stirred at 20° C. for 24 h. Upon completion, the reaction was quenched with saturated aq. NaHCO₃ to reach pH 7. The organic layer was dried over Na₂SO₄, filtered, and evaporated at low pressure. The residue was purified by flash silica gel chromatography (ISCO®; 12 g SepaFlash® Silica Flash Column, Eluent of 0˜7% DCM/MeOH gradient @ 20 mL/min) to give 1.6 g (impure, 75% LCMS purity), followed by prep-HPLC [FA condition, column: Boston Uni C18 40*150*5 um; mobile phase: [water (0.225% FA)-ACN]; B %: 8%-38%, 7.7 min.] to give 6 (1.04 g, 63.7% yield) as a white solid. ESI-LCMS: 485.0 [M+Na]⁺; ¹H NMR (400 MHz, DMSO-d₆) δ=11.27-11.21 (m, 1H), 8.77 (s, 1H), 8.74 (s, 1H), 8.05 (d, J=7.3 Hz, 2H), 7.68-7.62 (m, 1H), 7.59-7.53 (m, 2H), 7.39 (t, J=6.3 Hz, 1H), 6.16 (d, J=6.0 Hz, 1H), 5.48 (d, J=5.5 Hz, 1H), 4.55 (t, J=5.5 Hz, 1H), 4.43-4.37 (m, 1H), 4.08-4.02 (m, 1H), 3.41-3.36 (m, 1H), 3.35 (s, 3H), 3.31-3.22 (m, 1H), 2.91 (s, 3H).

Preparation of (Example 8 monomer): To a solution of 6 (1 g, 2.16 mmol) in DCM (30 mL) was added P1 (977.58 mg, 3.24 mmol, 1.03 mL), followed by DCI (306.43 mg, 2.59 mmol) at 0° C. in one portion under Ar atmosphere. The mixture was degassed and purged with Ar for 3 times, warmed to 20° C., and stirred for 2 hr under Ar atmosphere. Upon completion as monitored by LCMS and TLC (PE:EtOAc=4:1), the reaction mixture was diluted with sat.aq. NaHCO₃ (30 mL) and extracted with DCM (50 mL*2). The combined organic layers were dried over anhydrous Na₂SO₄, filtered, and the filtrate was concentrated under reduced pressure to give a residue. The crude product was purified by reversed-phase HPLC (40 g C18 column: neutral condition, Eluent of 0˜57% of 0.3% NH₄HCO₃ in H₂O/CH₃CN ether gradient @ 35 mL/min) to give Example 8 monomer (0.49 g, 33.7% yield) as a white solid. ESI-LCMS: 663.1 [M+H]⁺; ¹H NMR (400 MHz, CD₃CN) δ=1.19-1.29 (m, 12H) 2.71 (q, J=5.77 Hz, 2H) 2.94 (d, J=6.27 Hz, 3H) 3.35 (d, J=15.56 Hz, 3H) 3.40-3.52 (m, 2H) 3.61-3.97 (m, 4H) 4.23-4.45 (m, 1H) 4.55-4.74 (m, 2H) 6.02 (dd, J=10.67, 6.40 Hz, 1H) 7.25 (br s, 1H) 7.47-7.57 (m, 2H) 7.59-7.68 (m, 1H) 8.01 (d, J=7.78 Hz, 2H) 8.28 (s, 1H) 8.66 (s, 1H) 9.69 (br s, 1H); ³¹P NMR (162 MHz, CD₃CN) δ=150.92, 149.78.

### Example 9. Synthesis of 5′-Stabilized End Cap Modified Oligonucleotides

This example provides an exemplary method for synthesizing the siNAs comprising a 5′-stabilized end caps disclosed herein. The 5′-stabilized end cap and/or deuterated phosphoramidites were dissolved in anhydrous acetonitrile and oligonucleotide synthesis was performed on a Expedite 8909 Synthesizer using standard phosphoramidite chemistry. An extended coupling (12 minutes) of 0.12 M solution of phosphoramidite in anhydrous CH₃CN in the presence of Benzyl-thio-tetrazole (BTT) activator to a solid bound oligonucleotide followed by standard capping, oxidation and sulfurization produced modified oligonucleotides. The 0.02 M 12, THF: Pyridine; Water 7:2:1 was used as an oxidizing agent, while DDTT (dimethylamino-methylidene)amino)-3H-1,2,4-dithiazaoline-3-thione was used as the sulfur-transfer agent for the synthesis of oligoribonucleotide with a phosphorothioate backbone. The stepwise coupling efficiency of all modified phosphoramidites was achieved around 98%. After synthesis the solid support was heated with aqueous ammonia (28%) solution at 45° C. for 16 h or 0.05 M K₂CO₃ in methanol was used to deprotect the base labile protecting groups. The crude oligonucleotides were precipitated with isopropanol and centrifuged (Eppendorf 5810R, 3000 g, 4° C., 15 min) to obtain a pellet. The crude product was then purified using ion exchange chromatography (TSK gel column, 20 mM NaH₂PO₄, 10% CH₃CN, 1 M NaBr, gradient 20-60% 1 M NaBr over 20 column volumes) and fractions were analyzed by ion change chromatography on an HPLC. Pure fractions were pooled and desalted by Sephadex G-25 column and evaporated to dryness. The purity and molecular weight were determined by HPLC analysis and ESI-MS analysis. Single strand RNA oligonucleotides (sense and antisense strand) were annealed (1:1 by molar equivalents) at 90° C. for 3 min followed by RT 40 min) to produce the duplexes.

### Example 10. Synthesis of Monomer

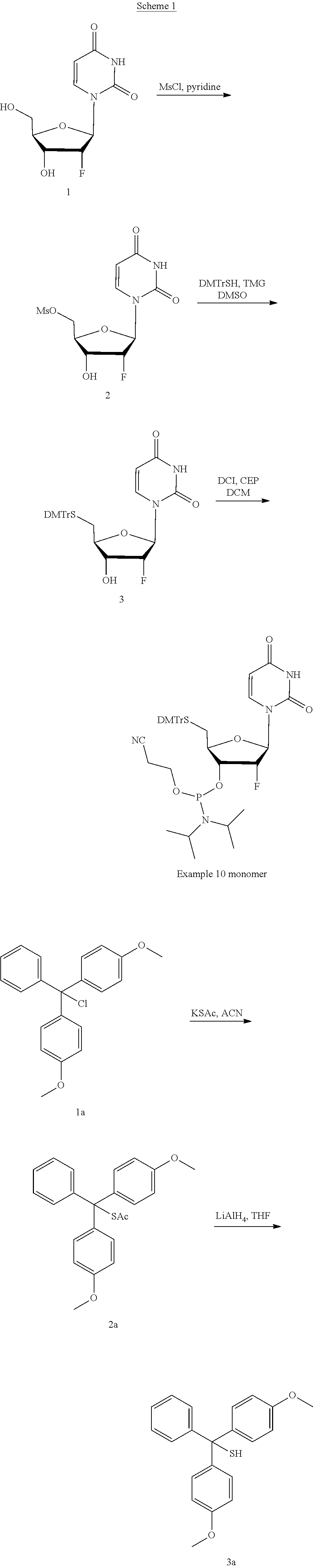

Preparation of (2a): To a solution of 1a (10.0 g, 29.5 mmol) in ACN (200.0 mL), KSAc (13.5 g, 118.6 mmol) was added at r.t., the mixture was stirred at r.t. for 15 h, TLC showed 1a was consumed completely. Mixture was filtered by silica gel and filter cake was washed with DCM (100.0 mL), the filtrate was concentrated to give crude 2a (11.1 g) as an oil. ¹H-NMR (400 MHz, CDCl₃): δ 7.32-7.24 (m, 5H), 7.16 (d, J=8.9 Hz, 4H), 6.82 (d, J=8.9 Hz, 4H), 3.82 (s, 6H), 2.28 (s, 3H).

Preparation of (3a): To a solution of crude 2a (11.1 g, 29.2 mmol) in THF (290.0 mL), LiAlH₄ (2.0 g, 52.6 mmol) was added at 0° C. and kept for 10 min, reaction was stirred at r.t. for 5 h under N₂, TLC showed 2a was consumed completely. Mixture was put into aqueous NaHCO₃ solution and extracted with EA (500.0 mL*2), organic phase was concentrated to give crude which was purified by column chromatography (SiO₂, PE/EA=30:1 to 10:1) to give 3a (8.1 g, 95% purity) as a white solid. ESI-LCMS: m/z 335.3 [M−H]⁻; ¹H-NMR (400 MHz, CDCl₃): δ 7.33-7.24 (m, 5H), 7.19 (d, J=8.8 Hz, 4H), 6.82 (d, J=8.8 Hz, 4H), 3.83 (s, 6H), 3.09 (s, 1H).

Preparation of (2): To a solution of 1 (20.0 g, 81.3 mmol) in pyridine (400.0 mL), MsCl (10.23 g, 89.43 mmol) was added dropwise at −10° C., reaction was stirred at −10° C. for 1 h, LCMS showed 1 was consumed completely, 100.0 mL aqueous NaHCO₃ solution was added and extracted with DCM (100.0 mL*2), organic phase was concentrated to give crude which was purified by column chromatography (SiO₂, DCM/MeOH=30:1 to 10:1) to give 2 (9.5 g, 97% purity) as a white solid. ESI-LCMS: m/z 325.3 [M+H]⁺; ¹H-NMR (400 MHz, DMSO-d₆): δ 11.45 (s, 1H), 7.64-7.62 (d, J=8.0 Hz, 1H), 5.92-5.85 (m, 2H), 5.65-5.63 (d, J=8.0 Hz, 1H), 5.26-5.11 (m, 1H), 4.53-4.37 (m, 2H), 4.27-4.16 (m, 1H), 4.10-4.04 (m, 1H), 3.23 (s, 3H).

Preparation of (3): Intermediate 3 was prepared by prepared according to reaction condition described in reference Helvetica Chimica Acta, 2004, 87. 2812. To a solution of 2 (9.2 g, 28.3 mmol) in dry DMSO (130.0 mL). DMTrSH (14.31 g, 42.5 mmol) was added, followed by tetramethylguanidine (3.6 g, 31.2 mmol) was added under N₂, reaction was stirred at r.t. for 3 h, LCMS showed 2 was consumed completely. 100.0 mL H₂O was added and extracted with EA (100.0 mL*2), organic phase was concentrated to give crude which was purified by column chromatography (SiO₂, PE/EA=5:1 to 1:1) to give 3 (12.0 g, 97% purity) as a white solid. ESI-LCMS: m/z 563.2 [M−H]⁻; ¹H-NMR (400 MHz, DMSO-d₆): δ 11.43-11.42 (d, J=4.0 Hz, 1H), 7.57-7.55 (d, J=8.0 Hz, 1H), 7.33-7.17 (m, 9H), 6.89-6.86 (m, 4H), 5.80-5.74 (m, 1H), 5.65-5.62 (m, 1H), 5.58-5.57 (d, J=4.0 Hz, 1H), 5.16-5.01 (m, 1H), 3.98-3.90 (m, 1H), 3.73 (s, 6H), 3.73-3.67 (m, 1H), 2.50-2.37 (m, 2H).

Preparation of Example 10 monomer: To a solution of 3 (10.0 g, 17.7 mmol) in dichloromethane (120.0 mL) with an inert atmosphere of nitrogen was added CEOP[N(iPr)₂]₂ (6.4 g, 21.2 mmol) and DCI (1.8 g, 15.9 mmol) in order at room temperature. The resulting solution was stirred for 1.0 h at room temperature and diluted with 50 mL dichloromethane and washed with 2×50 mL of saturated aqueous sodium bicarbonate and 1×50 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give Example 10 monomer (12.8 g, 98% purity, 93% yield) as an oil. ESI-LCMS: m/z 765.2 [M+H]⁺; ¹H-NMR (400 MHz, DMSO-d₆): δ 11.44 (s, 1H), 7.70-7.66 (m, 1H), 7.32-7.18 (m, 9H), 6.89-6.85 (m, 4H), 5.80-5.64 (m, 2H), 5.38-5.22 (m, 1H), 4.38-4.15 (m, 1H), 3.81-3.70 (m, 8H), 3.61-3.43 (m, 3H), 2.76-2.73 (m, 1H), 2.66-2.63 (m, 1H), 2.50-2.41 (m, 2H), 1.12-1.05 (m, 9H), 0.97-0.95 (m, 3H); ³¹P-NMR (162 MHz, DMSO-d₆): δ 149.01, 148.97, 148.74, 148.67; ¹⁹F-NMR (376 MHz, DMSO-d₆): δ 149.01, 148.97, 148.74, 148.67.

### Example 11. Synthesis of Monomer

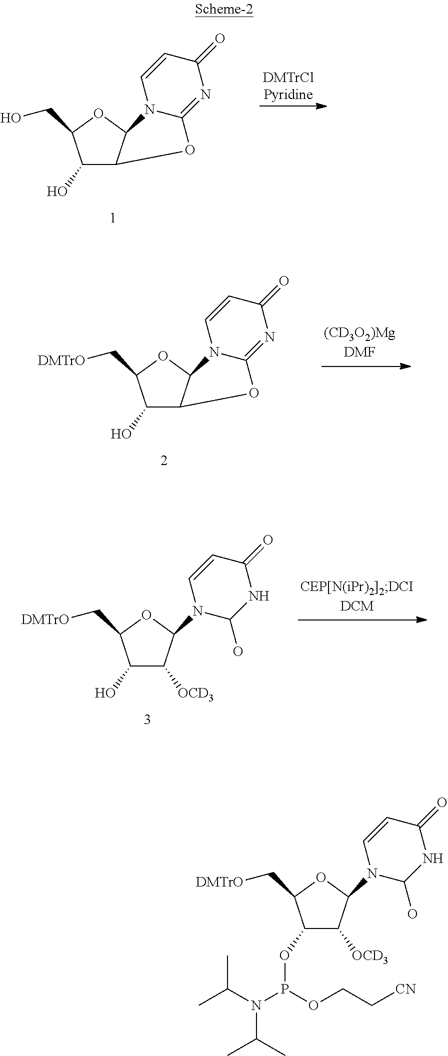

Preparation of (2): To a stirred solution of 1 (2.0 g, 8.8 mmol) in pyridine (20 mL) were added DMTrCl (3.3 g, 9.7 mmol) at r.t. The reaction mixture was stirred at r.t. for 2.5 hrs. With ice-bath cooling, the reaction was quenched with water and the product was extracted with EA (100 mL). The organic phase was evaporated to dryness under reduced pressure to give a residue which was purified by silica gel column chromatography (eluent, DCM:MeOH=50:1˜20:1) to give 2 (3.7 g, 7.2 mmol, 80.1%) as a white solid. ESI-LCMS: m/z 527 [M−H]⁻.

Preparation of (3): To the solution of 2 (2.8 g, 5.3 mmol) in dry DMF (56 mL) was added (CD₃O)₂ Mg (2.9 g, 31.8 mmol) at r.t. under N₂ atmosphere. The reaction mixture was stirred at 100° C. for 15 hrs. With ice-bath cooling, the reaction was quenched with saturated aq. NH₄Cl and extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (2.0 g, 3.6 mmol, 67.9%) as a white solid. ESI-LCMS: m/z 562 [M−H]⁻: ¹H-NMR (400 MHz, DMSO-d₆): δ 11.38 (s, 1H), 7.73 (d, J=8 Hz, 1H), 7.46-7.19 (m, 9H), 6.91 (d, J=7.4 Hz, 4H), 5.81-5.76 (AB, J=20 Hz, 1H), 5.30 (d, J=8 Hz, 1H), 5.22 (s, 1H), 4.25-4.15 (m, 1H), 3.99-3.92 (m, 1H), 3.85-3.79 (m, 1H), 3.74 (s, 6H), 3.34-3.18 (m, 31H).

Preparation of Example 11 monomer: To a suspension of 3 (2.0 g, 3.5 mmol) in DCM (20 mL) was added DCI (357 mg, 3.0 mmol) and CEP[N(iPr)₂]₂ (1.3 g, 4.3 mmol). The mixture was stirred at r.t. for 1 h. LC-MS showed 3 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 11 monomer (2.1 g, 2.7 mmol, 77.1%) as a white solid. ESI-LCMS: m/z 764 [M+H]⁺; ¹H-NMR (400 MHz, ACN-d₃): δ 9.45-8.90 (m, 1H, exchanged with D₂O), 7.88-7.66 (m, 1H), 7.50-7.18 (m, 9H), 6.93-6.80 (m, 4H), 5.85 (d, J=8.2 Hz, 1H), 5.29-5.16 (m, 1H), 4.57-4.37 (m, 1H), 4.18-4.09 (m, 1H), 3.98-3.90 (m, 1H), 3.90-3.74 (m, 7H), 3.74-3.50 (m, 3H), 3.48-3.31 (m, 2H), 2.70-2.61 (m, 1H), 2.56-2.46 (m, 1H), 1.24-1.12 (m, 9H), 1.09-0.99 (m, 3H). ³¹P-NMR (162 MHz, ACN-d₃): δ=149.87, 149.55.

### Example 12. Synthesis of Monomer

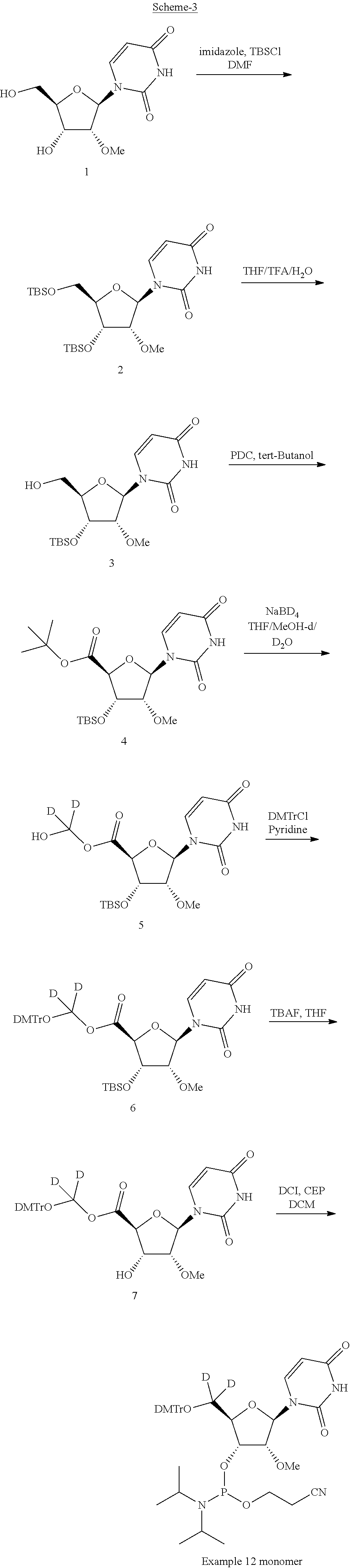

Preparation of (2): To the solution of 1 (39.2 g, 151.9 mmol) in DMF (390.0 mL) was added imidazole (33.0 g, 485.3 mmol) and TBSCl (57.2 g, 379.6 mmol) at 0° C. The reaction mixture was stirred at room temperature for 15 hrs under N₂ atmosphere. After addition of water, the resulting mixture was extracted with EA (500.0 mL). The combined organic layer was washed with water and brine, dried over Na₂SO₄, concentrated to give the crude 2 (85.6 g) as a white solid which was used directly for next step. ESI-LCMS: m/z 487.7 [M+H]⁺.

Preparation of (3): A solution of crude 2 (85.6 g) in a mixture solvent of TFA/H₂O=1/1 (400.0 mL) and THF (400.0 mL) was stirred at 0° C. for 30 min. After completion of reaction, the resulting mixture was added con. NH₃*H₂O to pH=7, and then extracted with EA (500.0 mL). The organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (36.6 g, 98.4 mmol, 64.7% over two step) as a white solid. ESI-LCMS: m/z 372.5 [M+H]⁺; ¹H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 3.68-3.52 (m, 2H), 0.88 (s, 9H), 0.09 (s, 6H).

Preparation of (4): To the solution of 3 (36.6 g, 98.4 mmol) in dry DCM (200.0 mL) and DMF (50.0 mL) was added PDC (73.9 g, 196.7 mmol), tert-butyl alcohol (188.0 mL) and Ac₂O (93.0 mL) at r.t under N₂ atmosphere, the reaction mixture was stirred at r.t for 2 hrs. The solvent was removed to give a residue which was purified by silica gel column chromatography (eluent, PE/EA=4:1 ˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (24.3 g, 54.9 mmol, 55.8%) as a white solid. ESI-LCMS: m/z 443.2 [M+H]⁺; ¹H-NMR (400 MHz, DMSO-d₆): δ 11.30 (d, J=1 Hz, 1H), 7.92 (d, J=8 Hz, 1H), 5.86 (d, J=6 Hz, 1H), 5.67-5.65 (m, 1H), 4.33-4.31 (m, 1H), 4.13 (d, J=3 Hz, 1H), 3.73-3.70 (m, 1H), 1.34 (s, 9H), 0.77 (s, 9H), 0.08 (s, 6H).

Preparation of (5): To the solution of 4 (18.0 g, 40.7 mmol) in dry THF/MeOD/D₂O=10/2/1 (145.0 mL) was added NaBD₄ (5.1 g, 122.1 mmol) three times during an hour at 50° C., the reaction mixture was stirred at r.t. for 2 hrs. After completion of reaction, adjusted pH value to 7 with CH₃COOD, after addition of water, the resulting mixture was extracted with EA (300.0 mL). The combined organic layer was washed with water and brine, dried over Na₂SO₄, concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (10.4 g, 27.8 mmol, 68.3%) as a white solid. ESI-LCMS: m/z 375.2 [M+H]⁺; ¹H-NMR (400 MHz, DMSO-d₆): δ 11.36 (d, J=1 Hz, 1H), 7.92 (d, J=8 Hz, 1H), 5.83 (d, J=5 Hz, 1H), 5.67-5.65 (m, 1H), 5.19 (s, 1H), 4.30 (t, J=5 Hz, 1H), 3.85-3.83 (m, 2H), 0.88 (s, 9H), 0.09 (s, 6H).

Preparation of (6): To a stirred solution of 5 (10.4 g, 27.8 mmol) in pyridine (100.0 mL) was added DMTrCl (12.2 g, 36.1 mmol) at r.t., The reaction mixture was stirred at r.t. for 2.5 hrs, the reaction was quenched with water and extracted with EA (200.0 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (13.5 g, 19.9 mmol, 71.6%) as a white solid. ESI-LCMS: m/z 677.8 [M+H]⁺; ¹H-NMR (400 MHz, DMSO-d₆): δ 11.39 (d, J=1 Hz, 1H), 7.86 (d, J=4 Hz, 1H), 7.35-7.21 (m, 9H), 6.90-6.88 (m, 4H), 5.78 (d, J=2 Hz, 1H), 5.30-5.27 (m, 1H), 4.33-4.30 (m, 1H), 3.91 (d, J=7 Hz, 1H), 3.85-3.83 (m, 1H), 3.73 (s, 6H), 3.38 (s, 3H), 0.77 (s, 9H), 0.03 (s, 3H), 0.01 (s, 3H).

Preparation of (7): To a solution of 6 (13.5 g, 19.9 mmol) in THF (130.0 mL) was added 1 M TBAF solution (19.0 mL). The reaction mixture was stirred at r.t. for 1.5 hrs. LC-MS showed 6 was consumed completely. Water (500.0 mL) was added and extracted with EA (300.0 mL), the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (10.9 g, 19.4 mmol, 97.5%) as a white solid. ESI-LCMS: m/z 563.6 [M+H]⁺; ¹H-NMR (400 MHz, DMSO-d₆): δ 11.39 (s, 1H), 7.23 (d, J=8 Hz, 1H), 7.73 (d, J=8 Hz, 1H), 7.36-7.23 (m, 9H), 6.90 (d, J=8 Hz, 4H), 5.81 (d, J=3 Hz, 1H), 5.30-5.28 (m, 1H), 5.22 (d, J=7 Hz, 1H), 4.20 (q, J=7 Hz, 1H), 3.93 (d, J=7 Hz, 1H), 3.81 (t, J=5 Hz, 1H), 3.74 (s, 6H), 3.41 (s, 3H).

Preparation of Example 12 monomer: To a suspension of 7 (10.9 g, 19.4 mmol) in DCM (100.0 mL) was added DCI (1.8 g, 15.7 mmol) and CEP[N(iPr)₂]₂ (6.1 g, 20.4 mmol). The mixture was stirred at r.t. for 1 h. LC-MS showed 7 was consumed completely.

The mixture was washed with water twice and brine, dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 12 monomer (12.5 g, 14.5 mmol, 74.7%) as a white solid. ESI-LCMS: m/z 863.6 [M+H]⁺; ¹H-NMR (400 MHz, DMSO-d₆): δ 11.39 (s, 1H), 7.81-7.55 (m, 1H), 7.40-7.22 (m, 9H), 6.92-6.87 (m, 4H), 5.83-5.80 (m, 1H), 5.32-5.25 (m, 1H), 4.46-4.34 (m, 1H), 4.10-3.98 (m, 2H), 3.84-3.73 (m, 7H), 3.60-3.50 (m, 3H), 3.42, 3.40 (s, 3H), 2.78 (t, J=6 Hz, 1H), 2.62-2.59 (m, 1H), 2.07 (s, 1H), 1.17-0.96 (m, 12H); ³¹P-NMR (162 MHz, DMSO-d₆): δ 149.37, 149.06.

### Example 13. Synthesis of Monomer

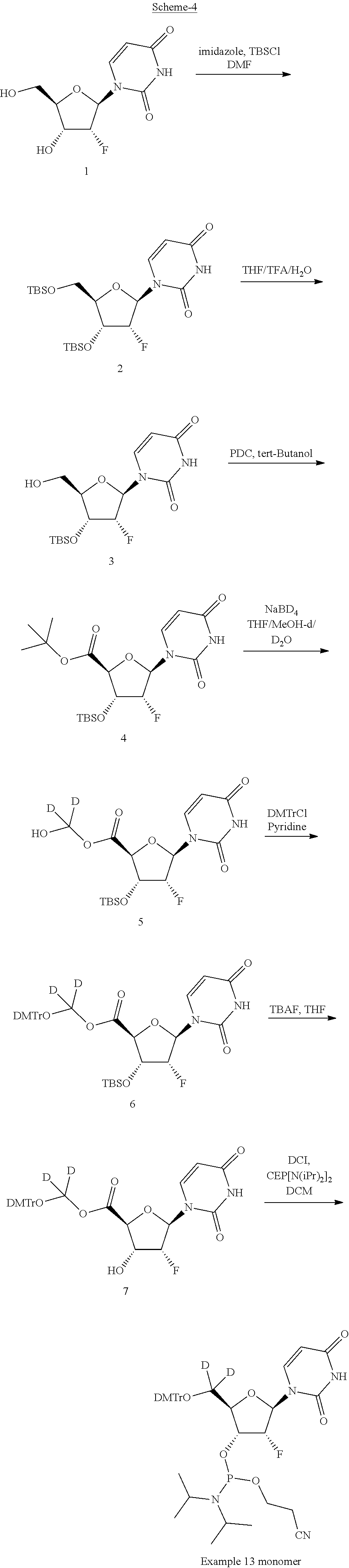

Preparation of (2): To the solution of 1 (13.0 g, 52.8 mmol) in DMF (100 mL) was added imidazole (12.6 g, 184.8 mmol) and TBSCl (19.8 g, 132.0 mmol) at 0° C., and the reaction mixture was stirred at room temperature for 15 h under N₂ atmosphere. After addition of water, the resulting product was extracted with EA (500 mL). The combined organic layer was washed with water and brine, dried over Na₂SO₄, and concentrated to give the crude 2 (30.6 g) as a white solid which was used directly for next step. ESI-LCMS: m/z 475 [M+H]⁺. WO2017106710A1

Preparation of (3): A solution of crude 2 (30.6 g) in a mixture solvent of TFA/H₂O=1/1 (100 mL) and THF (100 mL) was stirred at 0° C. for 30 min. After completion of reaction, the resulting mixture was added con. NH₃*H₂O to pH=7.5, and then the mixture was extracted with EA (500 mL), the organic layer was washed with brine, dried over Na₂SO₄ and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 3 (12.0 g, 33.3 mmol, 65.8% over two step) as a white solid. ESI-LCMS: m/z 361 [M+H]⁺; ¹H-NMR (400 MHz, DMSO-d₆): δ 11.39 (s, J=1 Hz, 1H, exchanged with D₂O), 7.88 (d, J=8 Hz, 1H), 5.91-5.86 (m, 1H), 5.66-5.62 (m, 1H), 5.21 (t, J=5.2 Hz, 1H, exchanged with D₂O), 5.18-5.03 (m, 1H), 4.37-4.29 (m, 1H), 3.87-3.83 (m, 1H), 3.78-3.73 (m, 1H), 3.56-3.51 (m, 1H), 0.87 (s, 9H), 0.09 (s, 6H). WO2017106710A1.

Preparation of (4): To the solution of 3 (11.0 g, 30.5 mmol) in dry DCM (60 mL) and DMF (15 mL) was added PDC (21. g, 61.0 mmol), tert-butyl alcohol (45 mL) and Ac₂O (32 mL) at r.t under N₂ atmosphere. And the reaction mixture was stirred at r.t for 2 h. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (9.5 g, 22.0 mmol, 72.3%) as a white solid. ESI-LCMS: m/z 431 [M+H]⁺; ¹H-NMR (400 MHz, DMSO-d₆): δ 11.45 (s, J=1 Hz, 1H, exchanged with D₂O), 7.93 (d, J=8.5 Hz, 1H), 6.02-5.97 (m, 1H), 5.76-5.74 (m, 1H), 5.29-5.14 (m, 1H), 4.59-4.52 (m, 1H), 4.29-4.27 (m, 1H), 1.46 (s, 9H), 0.89 (s, 9H), 0.12 (s, 6H).

Preparation of (5): To the solution of 4 (8.5 g, 19.7 mmol) in dry THF/MeOD/D₂O=10/2/1 (80 mL) was added NaBD₄ (2.5 g, 59.1 mmol) three times per an hour at 50° C. And the reaction mixture was stirred at r.t for 2 h. After completion of reaction, adjusted pH value to 7 with CH₃COOD, after addition of water, the resulting mixture was extracted with EA (300 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (3.5 g, 9.7 mmol, 50.3%) as a white solid. ESI-LCMS: m/z 363 [M+H]⁺; ¹H-NMR (400 MHz, DMSO-d₆): δ 11.41 (s, J=1 Hz, 1H, exchanged with D₂O), 7.88 (d, J=8 Hz, 1H), 5.91-5.86 (m, 1H), 5.66-5.62 (m, 1H), 5.19 (t, J=5.2 Hz, 1H, exchanged with D₂O), 5.18-5.03 (m, 1H), 4.37-4.29 (m, 1H), 3.87-3.83 (m, 1H), 0.88 (s, 9H), 0.10 (s, 6H).

Preparation of (6): To a stirred solution of 5 (3.4 g, 9.7 mmol) in pyridine (35 mL) were added DMTrCl (3.4 g, 10.1 mmol) at r.t. And the reaction mixture was stirred at r.t for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (PCT Int. Appl., 2019173602), (5.5 g, 8.3 mmol, 85.3%) as a white solid. ESI-LCMS: m/z 665 [M+H]⁺; ¹H-NMR (400 MHz, DMSO-d₆): δ 11.50 (d, J=1 Hz, 1H, exchanged with D₂O), 7.92 (d, J=4 Hz, 1H), 7.44-7.27 (m, 9H), 6.96-6.93 (m, 4H), 5.94 (d, J=20.5 Hz, 1H), 5.39-5.37 (m, 1H), 5.32-5.17 (m, 1H), 4.60-4.51 (m, 1H), 4.01 (d, J=8.8 Hz, 1H), 3.80 (s, 6H), 0.80 (s, 9H), 0.09 (s, 3H), −0.05 (s, 3H).

Preparation of (7): To a solution of 6 (5.5 g, 8.3 mmol) in THF (50 mL) was added 1 M TBAF solution (9 mL). The reaction mixture was stirred at r.t. for 1.5 h. LC-MS showed 6 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (4.1 g, 7.5 mmol, 90.0%) as a white solid. ESI-LCMS: m/z 551 [M+H]⁺; ¹H-NMR (400 MHz, DMSO-d₆): δ 11.42 (s, 1H, exchanged with D₂O), 7.76 (d, J=8.2 Hz, 1H), 7.39-7.22 (m, 9H), 6.90-6.88 (m, 4H), 5.83 (d, J=20.5 Hz, 1H), 5.65 (d, J=7.0 Hz, 1H, exchanged with D₂O), 5.29 (d, J=7.2 Hz, 1H), 5.18-5.03 (m, 1H), 4.40-4.28 (m, 1H), 4.01 (d, J=8.8 Hz, 1H), 3.74 (s, 6H).

Preparation of Example 13 monomer: To a suspension of 7 (4.1 g, 7.5 mmol) in DCM (40 mL) was added DCI (0.7 g, 6.4 mmol) and CEP[N(iPr)₂]₂ (2.9 g, 9.7 mmol). The mixture was stirred at r.t. for 1 h. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 13 monomer (5.0 g, 6.6 mmol, 90.0%) as a white solid. ESI-LCMS: m/z 751 [M+H]⁺; ¹H-NMR (400 MHz, DMSO-d₆): δ 11.43 (s, 1H), 7.85-7.82 (m, 1H), 7.40-7.23 (m, 9H), 6.90-6.85 (m, 4H), 5.94-5.86 (m, 1H), 5.40-5.24 (m, 2H), 4.74-4.49 (m, 1H), 4.12-4.09 (m, 2H), 3.79-3.47 (m, 10H), 2.78-2.59 (m, 2H), 1.14-0.93 (m, 12H). ³¹P-NMR (162 MHz, DMSO-d₆): δ 149.67, 149.61, 149.32, 149.27.

### Example 14. Synthesis of Monomer

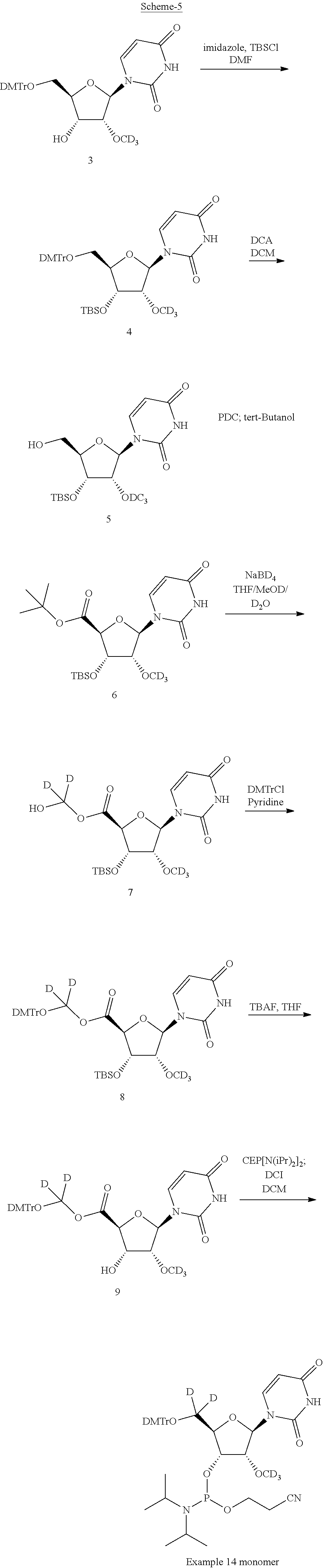

Preparation of (4): To the solution of 3 (14.3 g, 25.4 mmol, Scheme 2) in pyridine (150 mL) was added imidazole (4.5 g, 66.6 mmol) and TBSCl (6.0 g, 40.0 mmol) at 0° C., and the reaction mixture was stirred at room temperature for 15 h under N₂ atmosphere. After addition of water, the resulting mixture was extracted with EA (500 mL). The combined organic layer was washed with water and brine, dried over Na₂SO₄, and concentrated to give the crude 4 (18.0 g) as a white solid which was used directly for next step. ESI-LCMS: m/z 676 [M−H]⁻.

Preparation of (5): To the solution of crude 4 (18.0 g) in the solution of DCA (6%) in DCM (200 mL) was added TES (50 mL) at r.t, and the reaction mixture was stirred at room temperature for 5-10 min. After completion of reaction, the resulting mixture was added pyridine to pH=7, and then the solvent was removed and the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 5 (6.5 g, 17.2 mmol, 67.7% for two step) as a white solid. ESI-LCMS: m/z 376 [M+H]⁺; ¹H-NMR (400 MHz, DMSO-d₆): δ 7.92 (d, J=8 Hz, 1H), 5.82 (d, J=5.2 Hz, 1H), 5.68-5.63 (m, 1H), 5.20-5.15 (m, 1H), 4.32-4.25 (m, 1H), 3.87-3.80 (m, 2H), 3.69-3.61 (m, 1H), 3.57-3.49 (m, 1H), 0.88 (s, 9H), 0.09 (s, 6H).

Preparation of (6): To the solution of 5 (6.5 g, 17.2 mmol) in dry DCM (35 mL) and DMF (9 mL) was added PDC (12.9 g, 34.3 mmol), tert-butyl alcohol (34 mL) and Ac₂O (17 mL) at r.t under N₂ atmosphere. And the reaction mixture was stirred at r.t for 2 hrs. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (5.5 g, 12.3 mmol, 70.1%) as a white solid. ESI-LCMS: m/z 446 [M+H]⁺; ¹H-NMR (400 MHz, DMSO-d₆): δ=11.29 (s, 1H), 7.91 (d, J=8.4 Hz, 1H), 5.85 (d, J=6.4 Hz, 1H), 5.71-5.61 (m, 1H), 4.35-4.28 (m, 1H), 4.12 (d, J=3.2 Hz, 1H), 3.75-3.67 (m, 1H), 1.33 (s, 9H), 0.76 (s, 9H), 0.00 (d, J=1.6 Hz, 6H).

Preparation of (7): To the solution of 6 (5.4 g, 12.1 mmol) in THF/MeOD/D₂O=10/2/1 (44 mL) was added NaBD₄ (1.5 g, 36.3 mmol) at r.t. and the reaction mixture was stirred at 50° C. for 2 hrs. After completion of reaction, adjusted pH value to 7 with CH₃COOD. Water was added, the resulting mixture was extracted with EA (500 mL). The combined organic layer was washed with water and brine, dried over Na₂SO₄, and concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (2.6 g, 6.8 mmol, 56.1%) as a white solid. ESI-LCMS: m/z 378 [M+H]⁺; ¹H-NMR (400 MHz, DMSO-d₆): δ 11.35 (s, 1H), 7.91 (d, J=8.0 Hz, 1H), 5.82 (d, J=5.2 Hz, 1H), 5.69-5.60 (m, 1H), 5.14 (s, 1H), 4.34-4.20 (m, 1H), 3.88-3.76 (m, 2H), 0.87 (s, 9H), 0.08 (s, 6H).

Preparation of (8): To a stirred solution of 7 (2.6 g, 6.8 mmol) in pyridine (30 mL) were added DMTrCl (3.5 g, 10.3 mmol) at r.t. And the reaction mixture was stirred at r.t. for 2.5 hrs. With ice-bath cooling, the reaction was quenched with water and the product was extracted into EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (4.3 g, 6.3 mmol, 90.1%) as a white solid. ESI-LCMS: m/z 678 [M−H]⁻; ¹H-NMR (400 MHz, DMSO-d₆): δ 11.39 (s, 1H), 7.86 (d, J=8.0 Hz, 1H), 7.42-7.17 (m, 9H), 6.96-6.83 (m, 4H), 5.82-5.69 (m, 2H), 5.29 (d, J=8.4 Hz, 1H), 4.36-4.25 (m, 1H), 3.90 (d, J=7.2 Hz, 1H), 3.86-3.80 (m, 1H), 3.73 (s, 6H), 0.75 (s, 9H), 0.02 (s, 3H), −0.04 (s, 3H).

Preparation of (9): To a solution of 8 (4.3 g, 6.3 mmol) in THF (45 mL) was added 1 M TBAF solution (6 mL). The reaction mixture was stirred at r.t. for 1.5 hrs. LCMS showed 8 was consumed completely. Water (200 mL) was added. The product was extracted with EA (200 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 8 (3.5 g, 6.1 mmol, 90.1%) as a white solid. ESI-LCMS: m/z 678 [M−H]⁻; ¹H-NMR (400 MHz, DMSO-d₆): δ 11.38 (d, J=2.0 Hz, 1H), 7.23 (d, J=8.0 Hz, 1H), 7.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of Example 14 monomer: To a suspension of 9 (2.1 g, 3.7 mmol) in DCM (20 mL) was added DCI (373 mg, 3.1 mmol) and CEP[N(iPr)₂]₂ (1.3 g, 4.4 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 14 monomer (2.2 g, 3.5 mmol, 80%) as a white solid. ESI-LCMS: m/z 766 [M+H]⁺; ¹H-NMR (400 MHz, ACN-d₃): δ 9.65-8.86 (m, 1H, exchanged with D₂O), 7.93-7.68 (m, 1H), 7.52-7.19 (m, 9H), 6.94-6.78 (m, 4H), 5.95-5.77 (m, 1H), 5.31-5.17 (m, 1H), 4.61-4.37 (m, 1H), 4.20-4.07 (m, 1H), 4.01-3.51 (m, 10H), 2.74-2.59 (m, 1H), 2.57-2.43 (m, 1H), 1.27-1.10 (m, 9H), 1.09-0.95 (m, 3H). ³¹P-NMR (162 MHz, ACN-d₃): δ=149.88, 149.55.

### Example 15. Synthesis of Monomer

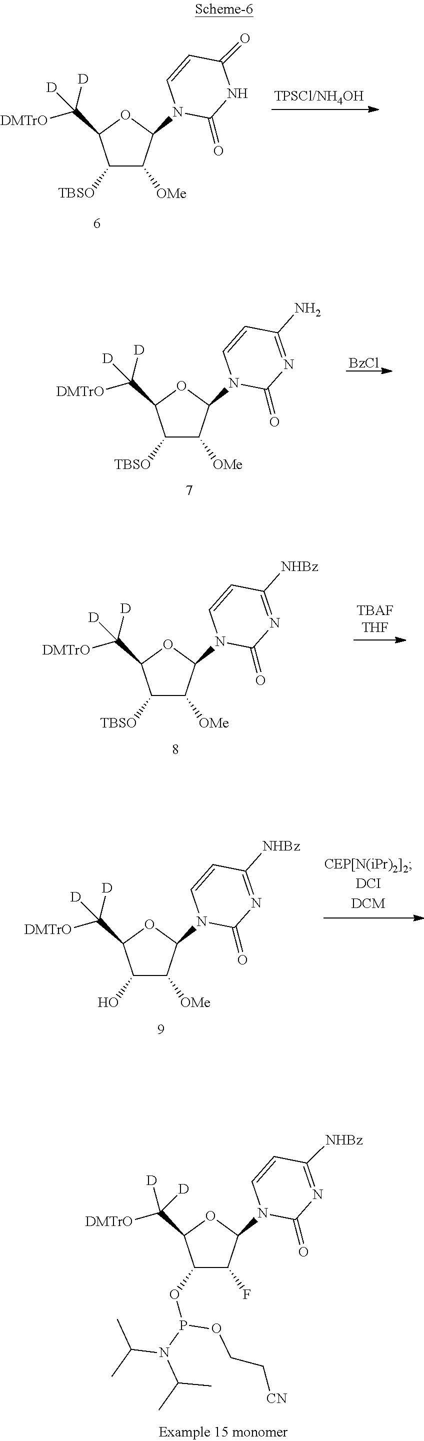

Preparation of (7): To a solution of 6 (17 g, 25.1 mmol, Scheme 3) in ACN (170 mL) was added DMAP (6.13 g, 50.3 mmol) and TEA (5.1 g, 50.3 mmol, 7.2 mL), Then added TPSCl (11.4 g, 37.7 mmol) at 0° C. under N₂ atmosphere and the mixture was stirred at r.t. for 3 h under N₂ atmosphere. Then con. NH₃·H₂O (27.3 g, 233.7 mmol) was added at r.t. and the mixture was stirred at r.t. for 16 h. The reaction was quenched with water and the product was extracted with EA (200 mL). The organic phase was concentrated to give the crude 7 (17.0 g) as a white solid which was used directly for next step.

Preparation of (8): To a stirred solution of 7 (17.0 g, 25.1 mmol) in pyridine (170 mL) were added BzCl (4.3 g, 30.1 mmol) 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (19.0 g, 24.3 mmol, 95.6% over two step) as a white solid. ESI-LCMS: m/z 780 [M+H]⁺.

Preparation of (9): To a solution of 8 (19.0 g, 24.3 mmol) in THF (190 mL) was added 1 M TBAF solution (24 mL). The reaction mixture was stirred at r.t. for 1.0 h. LC-MS showed 8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 9 (15.2 g, 23.1 mmol, 95.5%) as a white solid. ESI-LCMS: m/z 666 [M+H]⁺; ¹H-NMR (400 MHz, DMSO-d₆): δ 11.28 (s, 1H), 8.41 (m, 1H), 8.00-7.99 (m, 2H), 7.63-7.15 (m, 13H), 6.93-6.89 (m, 4H), 5.87 (s, 1H), 5.20 (d, J=7.4 Hz, 1H), 4.30 (m, 1H), 4.02 (m, 1H), 3.75 (s, 7H), 3.53 (s, 3H).

Preparation of Example 15 monomer: To a suspension of 9 (10.0 g, 15.0 mmol) in DCM (100 mL) was added DCI (1.5 g, 12.7 mmol) and CEP[N(iPr)₂]₂ (5.4 g, 18.0 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 15 monomer (11.5 g, 13.5 mmol, 90.7%) as a white solid. ESI-LCMS: m/z 866 [M+H]⁺; ¹H-NMR (400 MHz, DMSO-d₆): δ=11.28 (s, 1H), 8.48-8.41 (m, 1H), 8.00-7.99 (m, 2H), 7.63-7.11 (m, 13H), 6.93-6.89 (m, 4H), 5.92 (m, 1H), 4.55-4.44 (m, 1H), 4.17 (m, 1H), 3.95 (m, 1H), 3.80-3.62 (m, 7H), 3.57-3.46 (m, 5H), 3.32 (s, 1H), 2.78 (m, 1H), 2.62-2.59 (m, 1H), 1.19-0.94 (m, 12H); ³¹P-NMR (162 MHz, DMSO-d₆): δ=149.52, 148.82.

### Example 16. Synthesis of Monomer

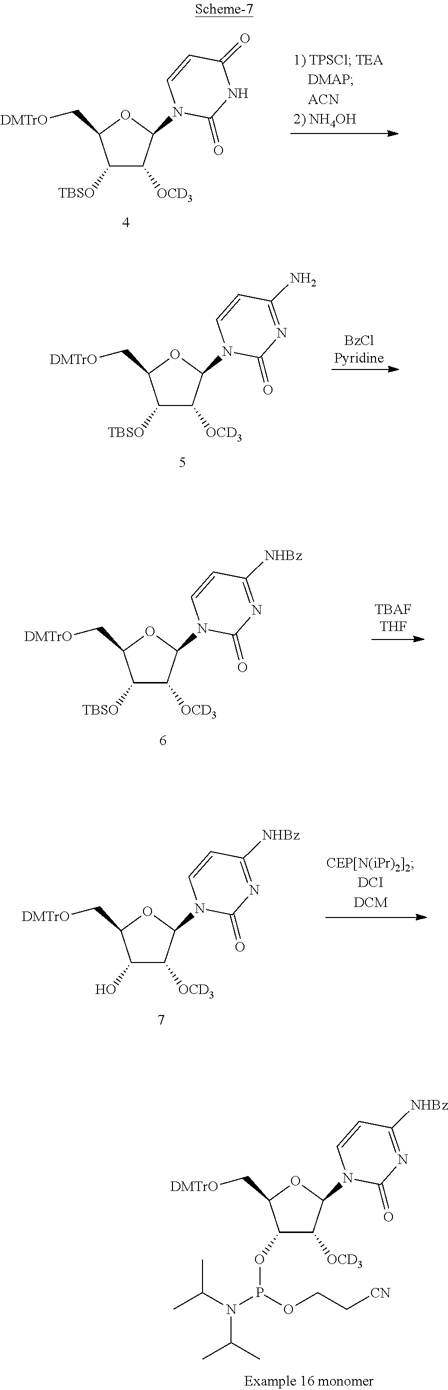

Preparation of (5): To the solution of 4 (18.8 g, Scheme 5) in dry ACN (200 mL) was added TPSCl (16.8 g, 65.2 mmol) and TEA (5.6 g, 65.2 mmol) and DMAP (6.8 g, 65.2 mmol), and the reaction mixture was stirred at room temperature for 3.5 hrs under N₂ atmosphere. After addition of water, the resulting mixture was extracted with EA (300 mL). The combined organic layer was washed with water and brine, dried over Na₂SO₄, and concentrated to give the crude 5 (22.0 g) as a white solid which was used directly for next step. ESI-LCMS: m/z 677 [M−H]⁺.

Preparation of (6): To a solution of 5 (22.0 g) in pyridine (150 mL) was added BzCl (6.8 g, 48.9 mmol) under ice bath. The reaction mixture was stirred at r.t. for 2.5 hrs. LCMS showed 5 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give the crude 6 (20.8 g, 26.7 mmol, 82% yield over two steps) as a white solid. ESI-LCMS: m/z 781 [M+H]⁺; ¹H-NMR (400 MHz, DMSO-d₆): δ 11.30 (s, 1H), 8.55 (d, J=8.0 Hz, 1H), 8.00-7.98 (m, 2H), 7.74-7.66 (m, 1H), 7.60-7.50 (m, 2H), 7.47-7.31 (m, 4H), 7.30-7.2 (m, 5H), 7.20-7.1 (m, 1H), 6.91 (d, J=7.4 Hz, 4H), 5.91-5.86 (AB, J=20.0 Hz, 1H), 4.30 (d, J=8.0 Hz, 1H), 3.87-3.78 (s, 1H), 3.78-3.70 (m, 6H), 3.62-3.51 (m, 1H), 3.28-3.2 (m, 1H), 2.15-2.05 (m, 3H), 0.73 (s, 9H), 0.00 (m, 6H).

Preparation of (7): To a solution of 6 (20.8 g, 26.7 mmol) in THF (210 mL) was added 1 M TBAF solution (32 mL). The reaction mixture was stirred at r.t. for 1.5 hrs. LCMS showed 6 was consumed completely. Water (600 mL) was added. The product was extracted with EA (400 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (12.4 g, 18.6 mmol, 70%) as a white solid. ESI-LCMS: m/z 667 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 7H), 2.57-2.42 (m, 2H).

Preparation of Example 16 monomer: To a suspension of 7 (12.4 g, 18.6 mmol) in DCM (120 mL) was added DCI (1.7 g, 15.8 mmol) and CEP[N(iPr)₂]₂ (7.3 g, 24.2 mmol). The mixture was stirred at r.t. for 2 hrs. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 16 monomer (13.6 g, 15.7 mmol, 84.0%) as a white solid. ESI-LCMS: m/z 867 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 10H), 2.74-2.59 (m, 1H), 2.57-2.43 (m, 1H), 1.27-1.10 (m, 9H), 1.09-0.95 (m, 3H). ³¹P-NMR (162 MHz, DMSO-d₆): δ 149.59, 148.85.

### Example 17. Synthesis of Monomer

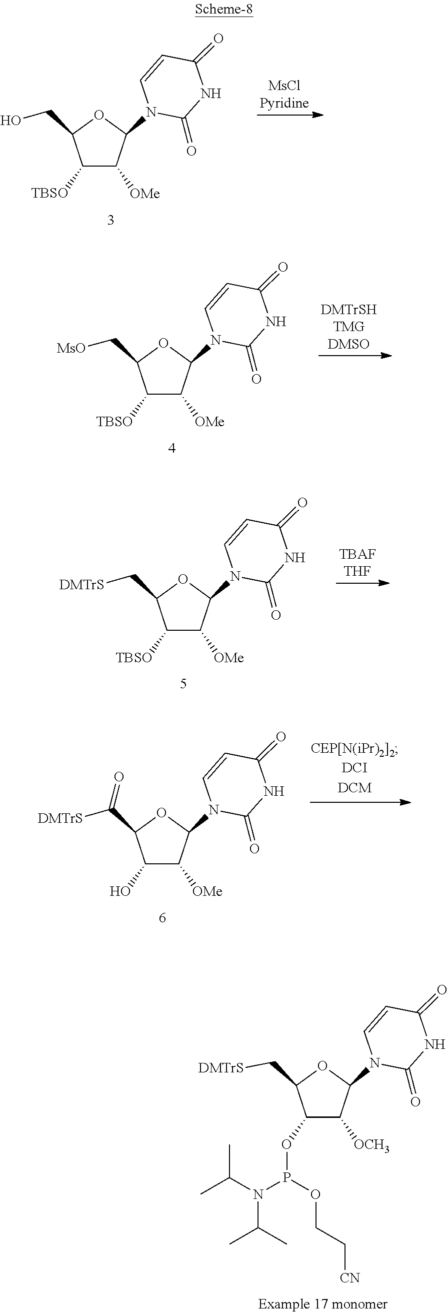

Preparation of (4): To a solution of 3 (13.1 g, 35.2 mmol, Scheme 3) in pyridine (130 mL) was added MsCl (4.8 g, 42.2 mmol) under-10˜0° C. The reaction mixture was stirred at r.t. for 2.5 h under N₂ atmosphere. TLC (DCM/MeOH=15:1) showed the reaction was consumed. The mixture was diluted with EA and water was added. The product was extracted with EA. The organic layer was washed with brine and dried over Na₂SO₄ and concentrated to give the crude. This resulted in to give the product 4 (14.2 g) which was used directly for the next step. ESI-LCMS: m/z 451 [M+H]⁺; ¹H-NMR (400 MHz, DMSO-d₆) δ 11.43 (m, 1H), 7.67-7.65 (m, 1H), 5.90-5.80 (m, 1H), 5.75-5.64 (m, 1H), 4.52-4.21 (m, 3H), 4.12-3.90 (m, 2H), 3.48-3.21 (m, 6H), 0.95-0.78 (s, 9H), 0.13-0.03 (s, 6H).

Preparation of (5): To a solution of 4 (14.2 g) in DMSO (200 mL) was added DMTrSH (19.6 g, 63.2 mmol) and tetramethylguanidine (5.1 g, 47.4 mmol) at r.t. The reaction mixture was stirred at r.t. for 3.5 h under N₂ atmosphere. LCMS showed 4 the reaction was consumed. The mixture was diluted with EA and water was added. The product was extracted with EA. The organic layer was washed with brine and dried over Na₂SO₄ and concentrated to give the crude. The crude was purified by silica gel column (SiO₂, PE/EA=10:1˜1:1) to give 5 (14.2 g, 20.6 mmol, 58.5% yield over two steps) as a white solid. ESI-LCMS: m/z 689 [M+H]⁻; ¹H-NMR (400 MHz, DMSO-d₆) δ 11.39 (m, 1H), 7.63-7.61 (d, J=8.0 Hz, 1H), 7.45-7.1 (m, 9H), 6.91-6.81 (m, 4H), 5.80-5.70 (m, 2H), 4.01-3.91 (m, 1H), 3.85-3.78 (m, 1H), 3.78-3.65 (m, 6H), 3.60-3.51 (m, 1H), 3.43-3.2 (m, 3H), 2.50-2.32 (m, 2H), 0.95-0.77 (s, 9H), −0.00-0.02 (s, 6H).

Preparation of (6): To a solution of 5 (14.2 g, 20.6 mmol) in THF (140 mL) was added 1 M TBAF solution (20 mL). The reaction mixture was stirred at r.t. under N₂ atmosphere for 2.5 h. LCMS showed 5 was consumed completely. Water was added. The product was extracted with EA and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 6 (10.5 g, 18.2 mmol, 88.5%) as a white solid. ESI-LCMS: m/z 576 [M+H]⁺; ¹H-NMR (400 MHz, DMSO-d₆) δ 11.38 (m, 1H), 7.56-7.54 (d, J=8.0 Hz, 1H), 7.45-7.1 (m, 9H), 6.91-6.81 (m, 4H), 5.80-5.70 (m, 2H), 4.05-4.00 (m, 1H), 3.81-3.79 (m, 1H), 3.74 (m, 2H), 3.78-3.65 (m, 6H), 3.60-3.51 (m, 1H), 3.43-3.2 (m, 3H), 2.40-2.32 (m, 1H).

Preparation of Example 17 monomer: To a suspension of 9 (10.5 g, 18.2 mmol) in DCM (100 mL) was added DCI (1.7 g, 15.5 mmol) and CEP[N(iPr)₂]₂ (7.2 g, 23.7 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 17 monomer (12.5 g, 16.1 mmol, 88%) as a white solid. ESI-LCMS: m/z 776 [M+H]⁺; ¹H-NMR (400 MHz, DMSO-d₆) δ 11.41 (m, 1H), 7.64-7.59 (m, 1H), 7.40-7.25 (m, 4H), 7.25-7.10 (m, 5H), 6.89-6.86 (m, 4H), 5.72-5.67 (m, 2H), 4.02-4.00 (m, 2H), 3.76-3.74 (m, 8H), 3.74-3.73 (m, 3H), 3.51-3.49 (d, J=8 Hz, 1H), 3.33-3.29 (m, 1H), 2.77-2.73 (m, 1H), 2.63-2.60 (m, 1H), 2.50-2.47 (m, 1H), 1.12-0.99 (m, 12H). ³¹P-NMR (162 MHz, DMSO-d₆): δ 148.92, 148.84.

### Example 18. Synthesis of Monomer

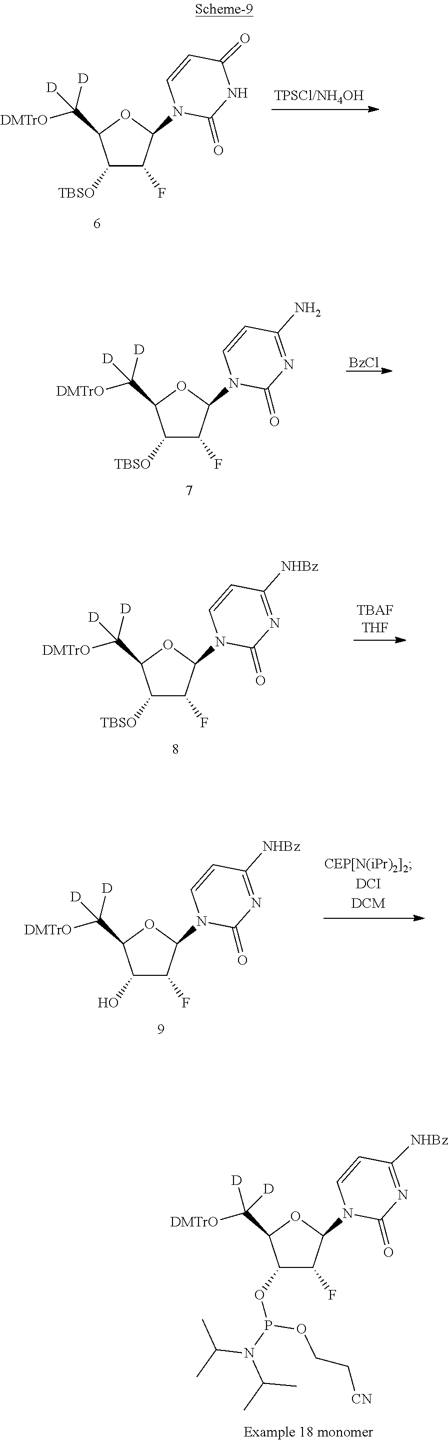

Preparation of (7): To a solution of 6 (16 g, 24.1 mmol, Scheme 4) in ACN (160 mL) was added DMAP (5.9 g, 48.2 mmol) and TEA (4.8 g, 48.2 mmol), then added TPSCl (10.9 g, 36.1 mmol) at 0° C. under N₂ atmosphere and the mixture was stirred at r.t. for 5 hrs under N₂ atmosphere. Then con. NH₃·H₂O (30 mL) was added at r.t. and the mixture was stirred at r.t. for 16 h. The reaction was quenched with water and the product was extracted with EA (200 mL). The organic phase was concentrated to give the crude 7 (16.0 g) as a white solid which was used directly for next step.

Preparation of (8): To a stirred solution of 7 (16.0 g, 24.1 mmol) in pyridine (160 mL) were added BzCl (4.1 g, 28.9 mmol) 0° C. under N₂ atmosphere. And the reaction mixture was stirred at r.t. for 2.5 h. With ice-bath cooling, the reaction was quenched with water and the product was extracted with EA (200 mL). The organic phase was evaporated to dryness under reduced pressure to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (18.0 g, 23.4 mmol, 97.0%) as a white solid. ESI-LCMS: m/z 768 [M+H]⁺; ¹H-NMR (400 MHz, DMSO-d₆): δ 11.31 (s, 1H), 8.47 (d, J=7.2 Hz, 1H), 7.99 (d, J=7.6 Hz, 2H), 7.65-7.16 (m, 13H), 6.92 (d, J=8.8 Hz, 4H), 6.01 (d, J=18.4 Hz, 1H), 5.18-5.04 (dd, 1H), 4.58-4.52 (m, 1H), 4.07 (d, J=9.6 Hz, 1H), 3.75 (s, 6H), 0.73 (s, 9H), 0.05 (s, 3H), −0.06 (s, 3H).

Preparation of (9): To a solution of 8 (18.0 g, 23.4 mmol) in THF (180 mL) was added 1 M TBAF solution (23 mL). The reaction mixture was stirred at r.t. for 1.5 h. LC-MS showed 8 was consumed completely. Water (500 mL) was added. The product was extracted with EA (300 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 7 (13.7 g, 21.1 mmol, 90.5%) as a white solid. ESI-LCMS: m/z 654.2 [M+H]⁺; ¹H-NMR (400 MHz, DMSO-d₆): δ 11.31 (s, 1H), 8.35 (d, J=7.4 Hz, 1H), 8.01 (m, 2H), 7.65-7.16 (m, 13H), 6.92 (d, J=8.8 Hz, 4H), 5.94 (d, J=18.0 Hz, 1H), 5.71 (d, J=7.0 Hz, 1H), 5.12-4.98 (dd, 1H), 4.51-4.36 (m, 1H), 4.09 (d, J=9.6 Hz, 1H), 3.75 (s, 6H).

Preparation of Example 18 monomer: To a suspension of 9 (10.6 g, 16.2 mmol) in DCM (100 mL) was added DCI (1.6 g, 13.7 mmol) and CEP[N(iPr)₂]₂ (5.8 g, 19.4 mmol). The mixture was stirred at r.t. for 1 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 18 monomer (10.5 g, 14.5 mmol, 75.9%) as a white solid. ESI-LCMS: m/z 854.3 [M+H]⁺; ¹H-NMR (400 MHz, DMSO-d₆): δ 11.31 (s, 1H), 8.41-8.37 (m, 1H), 8.01 (d, J=7.7 Hz, 2H), 7.65-7.16 (m, 13H), 6.92-6.88 (m, 4H), 6.06-5.98 (m, 1H), 5.33-5.15 (m, 1H), 4.78-4.58 (m, 1H), 4.23-4.19 (m, 1H), 3.81-3.73 (m, 6H), 3.60-3.50 (m, 3H), 3.32 (s, 1H), 2.76 (t, J=6.0 Hz, 1H), 2.60 (t, J=5.8 Hz, 1H), 1.15-0.94 (m, 12H); ³¹P-NMR (162 MHz, DMSO-d₆): δ 150.23, 150.18, 149.43, 149.38.

### Example 19. Synthesis of Monomer

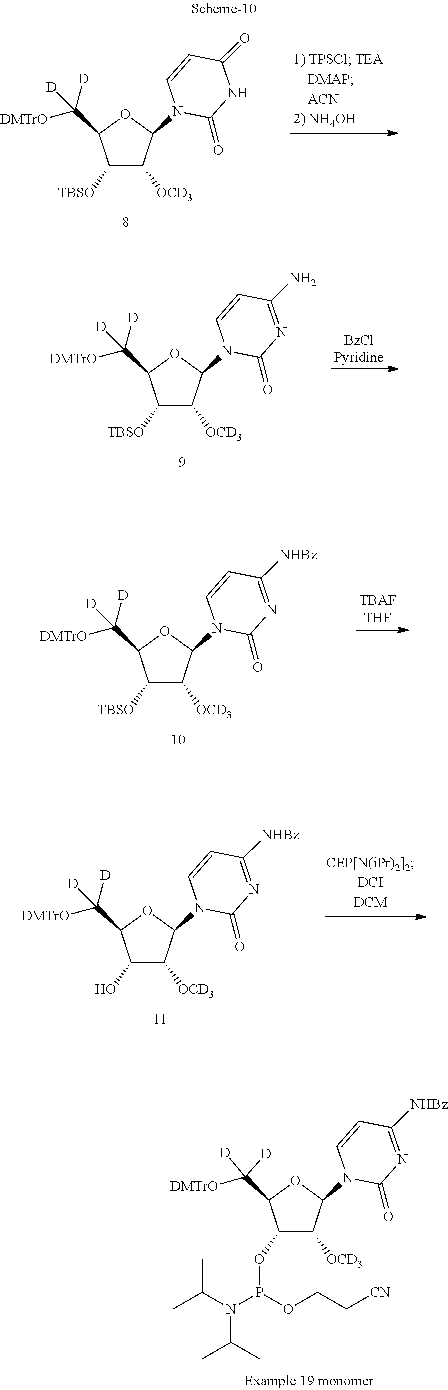

Preparation of (9): To a solution of 8 (18.8 g, 26.4 mmol, Scheme 5) in ACN (200 mL) was added TPSCl (16.8 g, 55.3 mmol) and DMAP (5.6 g, 55.3 mmol) and TEA (6.8 g, 55.3 mmol). The reaction mixture was stirred at r.t. for 3.5 hrs. LCMS showed the reaction was consumed. The mixture was diluted with con. NH₄OH (28 mL). The mixture was diluted with water and EA. The product was extracted with EA. The organic layer was washed with brine and dried over Na₂SO₄ and concentrated to give the crude 9 (18.5 g) which was used directly for the next step.

Preparation of (10): To a solution of 9 (18.8 g, 27.69 mmol) in pyridine (200 mL) was added BzCl (5.8 g, 41.5 mmol) under ice bath. The reaction mixture was stirred at r.t. for 2.5 hrs. LCMS showed 9 was consumed. The mixture was diluted with EA and water was added. The product was extracted with EA. The crude was purified by Flash-Prep-HPLC with the following conditions (IntelFlash-1): Column, C18 silica gel; mobile phase,

CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give 10 (19.8 g, 25.3 mmol, 91% yield) as a white solid. ESI-LCMS: m/z 783 [M−H]⁻; ¹H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of (11): To a solution of 10 (18.8 g, 26.4 mmol) in THF (190 mL) was added 1 M TBAF solution (28 mL). The reaction mixture was stirred at r.t. for 1.5 hrs. LCMS showed 10 was consumed completely. Water (200 mL) was added. The product was extracted with EA (200 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 11 (17.1 g, 25.6 mmol, 96%) as a white solid. ESI-LCMS: m/z 669 [M−H]⁻; ¹H-NMR (400 MHz, DMSO-d₆): δ 11.29 (d, J=2.0 Hz, 1H), 8.42 (d, J=8.0 Hz, 1H), 8.02-8.00 (m, 2H), 7.64-7.62 (m, 1H), 7.60-7.41 (m, 2H), 7.47.41-7.19 (m, 9H), 6.94-6.85 (m, 4H), 5.81 (d, J=4.0 Hz, 1H), 5.33-5.26 (m, 1H), 5.21 (d, J=7.2 Hz, 1H), 4.06-3.90 (m, 2H), 3.83-3.77 (m, 1H), 3.74 (s, 6H).

Preparation of Example 19 monomer: To a suspension of 11 (10.8 g, 16.2 mmol) in DCM (100 mL) was added DCI (1.5 g, 13.7 mmol) and CEP[N(iPr)₂]₂ (5.8 g, 19.3 mmol). The mixture was stirred at r.t. for 2 hrs. LC-MS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 19 monomer (11.3 g, 13 mmol, 80%) as a white solid. ESI-LCMS: m/z 868 [M+H]⁺; ¹H-NMR (400 MHz, DMSO-d₆): δ 11.03 (m, 1H), 8.51-8.48 (m, 1H), 8.08-7.95 (m, 2H), 7.63-7.54 (m, 1H), 7.52-7.19 (m, 9H), 7.16-7.07 (m, 1H), 6.94-6.89 (m, 3H), 5.95-5.87 (m, 1H), 5.31-5.17 (m, 1H), 4.61-4.37 (m, 1H), 4.20-4.07 (m, 1H), 3.82-3.47 (m, 10H), 2.74-2.59 (m, 1H), 2.57-2.43 (m, 1H), 1.27-1.10 (m, 9H), 1.09-0.95 (m, 3H). ³¹P-NMR (162 MHz, DMSO-d₆): δ 149.52, 148.81.

### Example 20. Synthesis of Monomer

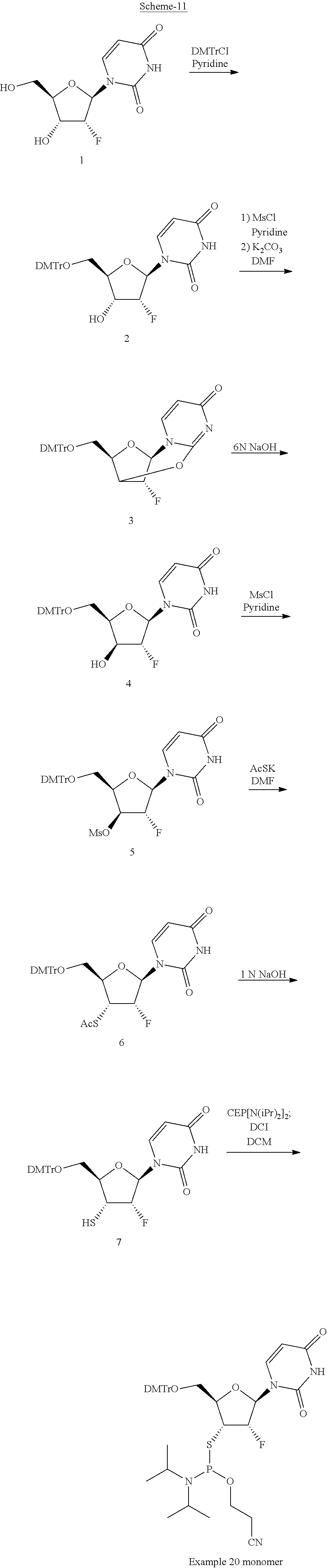

Preparation of (2): To a stirred solution of 1 (100.0 g, 406.5 mmol) in pyridine (1000 mL) were added DMTrCl (151.2 g, 447.1 mmol) at r.t. And the reaction mixture was stirred at r.t. for 2.5 hrs. With ice-bath cooling, the reaction was quenched with water and the product was extracted with EA (3000 mL). The organic phase was evaporated to dryness under reduced pressure to give a residue which was purified by silica gel column chromatography (SiO₂, dichloromethane:methanol=100:1) to give 2 (210.0 g, 90%) as a white solid. ESI-LCMS: m/z 548.2 [M+H]⁺; ¹H-NMR (400 MHz, DMSO-d₆): δ 11.43 (d, J=1.8 Hz, 1H), 7.77 (d, J=8.0 Hz, 1H), 7.40-7.21 (m, 9H), 6.92-6.88 (m, 4H), 5.89 (d, J=20.0 Hz, 1H), 5.31-5.29 (m, 1H), 5.19-5.04 (dd, 1H), 4.38-4.31 (m, 1H), 4.02-3.98 (m, 1H), 3.74 (s, 6H), 3.30 (d, J=3.2 Hz, 2H); ¹⁹F-NMR (376 MHz, DMSO-d₆): δ −199.51.

Preparation of (3): To a stirred solution of 2 (100.0 g, 182.8 mmol) in pyridine (1000 mL) were added MsCl (31.2 g, 274.2 mmol) at 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give the crude (114.0 g) as a white solid which was used directly for next step. To the solution of the crude (114.0 g, 187.8 mmol) in DMF (2000 mL) was added K₂CO₃ (71.5 g, 548.4 mmol), and the reaction mixture was stirred at 90° C. for 15 h under N₂ atmosphere. After addition of water, the resulting mixture was extracted with EA (500 mL). The combined organic layer was washed with water and brine, dried over Na₂SO₄, and concentrated to give a residue which was purified by silica gel column chromatography (SiO₂, dichloromethane:methanol=30:1) to give 3 (100.0 g, 90%) as a white solid. ESI-LCMS: m/z 531.2 [M+H]⁺; ¹H-NMR (400 MHz, DMSO-d₆): δ 7.79 (d, J=8.0 Hz, 1H), 7.40-7.21 (m, 9H), 6.89-6.83 (m, 4H), 6.14 (d, J=5.4 Hz, 1H), 6.02-5.90 (dd, 1H), 5.87 (d, J=20.0 Hz, 1H), 5.45 (m, 1H), 4.61 (m, 1H), 3.73 (d, J=1.9 Hz, 6H), 3.30-3.15 (m, 2H), 1.24-1.16 (m, 1H); ¹⁹F-NMR (376 MHz, DMSO-d₆): δ −204.23.

Preparation of (4): A solution of 3 (100 g, 187.8 mmol) in THF (1000 mL) was added 6N NaOH (34 mL, 206.5 mmol). The mixture was stirred at r.t. for 6 h. After completion of reaction, the resulting mixture was added H₂O, and then the mixture was extracted with EA, the organic layer was washed with brine, dried over sodium sulfate and removed to give the residue was purified by silica gel column chromatography (SiO₂, dichloromethane:methanol=30:1) to give 4 (90.4 g, 90%) as a white solid. ESI-LCMS: m/z 548.2 [M+H]⁺; ¹⁹F-NMR (376 MHz, DMSO-d₆): δ −184.58.

Preparation of (5): To a stirred solution of 4 (90.4 g, 165.2 mmol) in pyridine (1000 mL) were added MsCl (61.5 g, 495.6 mmol) at 0° C. under N₂ atmosphere. And the reaction mixture was stirred at r.t for 16 hrs. With ice-bath cooling, the reaction was quenched with water and the product was extracted with EA. the organic layer was washed with brine, dried over sodium sulfate and removed to give the residue was purified by silica gel column chromatography (SiO₂, PE:EA=1:1) to give 5 (75.0 g, 90%) as a white solid. ESI-LCMS: m/z 626.2 [M+H]⁺; ¹H-NMR (400 MHz, DMSO-d₆): δ 11.51 (d, J=1.6 Hz, 1H), 7.43-7.23 (m, 10H), 6.92-6.88 (m, 4H), 6.08 (d, J=20.0 Hz, 1H), 5.55-5.39 (m, 2H), 4.59 (m, 1H), 3.74 (s, 6H), 3.48-3.28 (m, 2H), 3.17 (s, 3H); ¹⁹F-NMR (376 MHz, DMSO-d₆): δ −187.72.

Preparation of (6): To the solution of 5 (75.0 g, 120.4 mmol) in DMF (1500 mL) was added KSAc (71.5 g, 548.4 mmol) at 110° C. under N₂ atmosphere, After the reaction mixture was stirred at 110° C. for 3 h were added KSAc (71.5 g, 548.4 mmol) under N₂ atmosphere. And the reaction mixture was stirred at r.t for 16 h. After addition of water, the resulting mixture was extracted with EA. The combined organic layer was washed with water and brine, dried over Na₂SO₄, and concentrated to give a residue which was purified by silica gel column chromatography (SiO₂, PE:EA=1:1) to give 6 (29.0 g, 90%) as a white solid. ESI-LCMS: m/z 605.2 [M+H]⁺; ¹H-NMR (400 MHz, DMSO-d₆): δ 11.45 (d, J=1.9 Hz, 1H), 7.95 (d, J=8.0 Hz, 1H), 7.38-7.21 (m, 9H), 6.92-6.87 (m, 4H), 5.93 (m, 1H), 5.50-5.36 (dd, 1H), 5.25-5.23 (dd, 1H), 4.54-4.42 (m, 1H), 4.17-4.12 (m, 1H), 3.74 (m, 7H), 3.35-3.22 (m, 2H), 2.39 (s, 1H); ¹⁹F-NMR (376 MHz, DMSO-d₆): δ −181.97.

Preparation of (7): A solution of 6 (22 g, 36.3 mmol) in a mixture solvent of THF/MeOH (1:1, 200 mL) was added IN NaOMe (70 mL, 72.6 mmol) was stirred at 20° C. for 4 h. After completion of reaction, the resulting mixture was added H₂O, and then the mixture was extracted with EA, the organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 25 min, the eluted product was collected at CH₃CN/H₂O (0.5% NH₄HCO₃)=4/3; Detector, UV 254 nm. This resulted in to give 7 (10.5 g, 14.5 mmol, 75.9%) as a white solid. ESI-LCMS: m/z 565.1 [M+H]⁺; ¹H-NMR (400 MHz, DMSO-d₆): δ 11.45 (s, 1H), 7.83 (d, J=8.0 Hz, 1H), 7.40-7.23 (m, 9H), 6.90 (d, J=8.8 Hz, 4H), 5.88 (m, 1H), 5.29-5.15 (m, 2H), 3.72 (m, 7H), 3.43 (m, 2H), 2.78 (d, J=10.6 Hz, 1H).

Preparation of Example 20 monomer: To a suspension of 7 (10.5 g, 18.6 mmol) in DCM (100 mL) was added DCI (1.8 g, 15.7 mmol) and CEP[N(iPr)₂]₂ (6.7 g, 22.3 mmol). The mixture was stirred at r.t. for 1 h. LC-MS showed 8 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 20 monomer (10.5 g, 14.5 mmol, 75.9%) as a white solid. ESI-LCMS: m/z 765.3 [M+H]⁺; ¹H-NMR (400 MHz, DMSO-d₆): δ 11.40 (d, J=12.2 Hz, 1H), 7.90-7.86 (m, 1H), 7.41-7.24 (m, 9H), 6.91-6.89 (m, 4H), 5.97 (m, 1H), 5.33-5.10 (m, 2H), 4.18-4.16 (m, 1H), 3.91-3.39 (m, 17H), 2.81 (t, J=5.6 Hz, 1H), 2.66 (t, J=6.0 Hz, 1H), 1.33-0.97 (m, 12H); ³¹P-NMR (162 MHz, DMSO-d₆): δ 164.57, 160.13.

### Example 21. Synthesis of Monomer

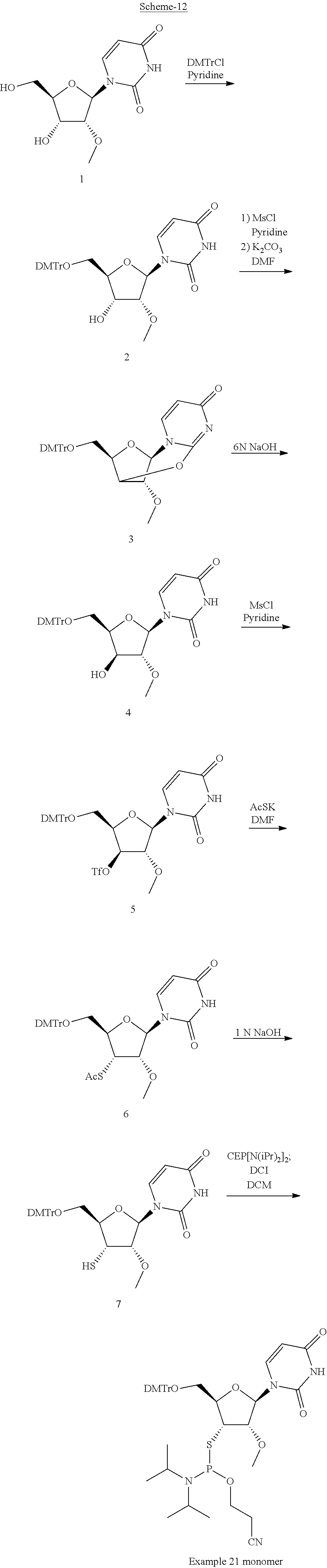

Preparation of (2): To a stirred solution of 1 (100.0 g, 387.5 mmol) in pyridine (1000 mL) was added DMTrCl (151.2 g, 447.1 mmol) at r.t. And the reaction mixture was stirred at r.t. for 2.5 hrs. With ice-bath cooling, the reaction was quenched with water and the product was extracted with EA (3000 mL). The organic phase was evaporated to dryness under reduced pressure to give a residue which was purified by silica gel column chromatography (SiO₂, dichloromethane:methanol=100:1) to give 2 (200.0 g, 90%) as a white solid. ESI-LCMS: m/z 561 [M+H]⁺.

Preparation of (3): To a stirred solution of 2 (73.0 g, 130.3 mmol) in pyridine (730 mL) were added MsCl (19.5 g, 169.2 mmol) at 0° C. under N₂ atmosphere. And the reaction mixture was stirred at r.t for 2.5 h. With ice-bath cooling, the reaction was quenched with water and the product was extracted with EA (200 mL). The organic phase was evaporated to dryness under reduced pressure to give the crude (80.0 g) as a white solid which was used directly for next step. To the solution of the crude (80.0 g, 130.3 mmol) in DMF (1600 mL) was added K₂CO₃ (71.5 g, 390.9 mmol), and the reaction mixture was stirred at 90° C. for 15 h under N₂ atmosphere. After addition of water, the resulting mixture was extracted with EA (500 mL). The combined organic layer was washed with water and brine, dried over Na₂SO₄, and concentrated to give a residue which was purified by silica gel column chromatography (SiO₂, dichloromethane:methanol=30:1) to give 3 (55.0 g, 90%) as a white solid. ESI-LCMS: m/z 543. [M+H]⁺; ¹H-NMR (400 MHz, DMSO-d₆): δ 7.68 (d, J=8.0 Hz, 1H), 7.40-7.21 (m, 9H), 6.89-6.83 (m, 4H), 5.96 (s, 1H), 5.83 (d, J=5.4 Hz, 1H), 5.26 (s, 1H), 4.59 (s, 1H), 4.46 (t, J=6.0 Hz, 1H), 3.72 (s, 6H), 3.44 (s, 3H), 3.18-3.12 (m, 2H).

Preparation of (4): A solution of 3 (55 g, 101.8 mmol) in THF (550 mL) was added 6N NaOH (34 mL, 206.5 mmol). The mixture was stirred at 20° C. for 6 hrs. After completion of reaction, the resulting mixture was added H₂O, and then the mixture was extracted with EA, the organic layer was washed with brine, dried over sodium sulfate and removed to give the residue was purified by silica gel column chromatography (SiO₂, dichloromethane:methanol=30:1) to give 4 (57.4 g, 87%) as a white solid. ESI-LCMS: m/z 561 [M+H]⁺.

Preparation of (5): To a stirred solution of 4 (57.4 g, 101.8 mmol) in pyridine (550 mL) were added MsCl (61.5 g, 495.6 mmol) at 0° C. under N₂ atmosphere. And the reaction mixture was stirred at r.t for 16 h. With ice-bath cooling, the reaction was quenched with water and the product was extracted with EA. the organic layer was washed with brine, dried over sodium sulfate and removed to give the residue was purified by silica gel column chromatography (SiO₂, PE:EA=1:1) to give 5 (57.0 g, 90%) as a white solid. ESI-LCMS: m/z 639 [M+H]⁺.

Preparation of (6): To the solution of 5 (57.0 g, 89.2 mmol) in DMF (600 mL) was added KSAc (71.5 g, 448.4 mmol) at 110° C. under N₂ atmosphere, After the reaction mixture was stirred at 110° C. for 3 h were added KSAc (71.5 g, 448.4 mmol) under N₂ atmosphere. And the reaction mixture was stirred at r.t for 16 h. After addition of water, the resulting mixture was extracted with EA. The combined organic layer was washed with water and brine, dried over Na₂SO₄, and concentrated to give a residue which was purified by silica gel column chromatography (SiO₂, PE:EA=1:1) to give 6 (29.0 g, 47%) as a white solid. ESI-LCMS: m/z 619.2 [M+H]⁺; ¹H-NMR (400 MHz, DMSO-d₆): δ 11.41 (s, 1H), 8.06 (s, 1H), 7.40-7.23 (m, 9H), 6.90 (d, J=8.8 Hz, 4H), 5.82 (s, 1H), 5.10-5.08 (dd, 1H), 4.38-4.34 (m, 1H), 4.08-4.02 (m, 3H), 3.74 (s, 6H), 3.45 (s, 3H), 3.25 (m, 2H), 2.37 (s, 3H); ESI-LCMS: m/z 619 [M+H]⁺.

Preparation of (7): A solution of 6 (22 g, 35.3 mmol) in a mixture solvent of THF/MeOH (1:1, 200 mL) was added IN NaOMe (70 mL, 72.6 mmol) was stirred at 20° C. for 4 h. After completion of reaction, the resulting mixture was added H₂O, and then the mixture was extracted with EA, the organic layer was washed with brine, dried over sodium sulfate and removed to give the residue was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 25 min, the eluted product was collected at CH₃CN/H₂O (0.5% NH₄HCO₃)=4/3; Detector, UV 254 nm. This resulted in to give 7 (14.0 g, 70.9%) as a white solid. ESI-LCMS: m/z 576.1 [M+H]⁺; ¹H-NMR (400 MHz, DMSO-d₆): δ 11.38 (s, 1H), 7.90 (d, J=8.0 Hz, 1H), 7.40-7.23 (m, 9H), 6.90 (d, J=8.8 Hz, 4H), 5.80 (s, 1H), 5.15-5.13 (dd, 1H), 3.93 (m, 1H), 3.87 (d, J=5.0 Hz, 1H), 3.74 (s, 6H), 3.59 (m, 2H), 3.49 (s, 3H), 3.39 (d, J=2.2 Hz, 2H), 2.40 (d, J=10.2 Hz, 1H).

Preparation of Example 21 monomer: To a suspension of 7 (10.5 g, 18.6 mmol) in DCM (100 mL) was added DCI (1.8 g, 15.7 mmol) and CEP[N(iPr)₂]₂ (6.7 g, 22.3 mmol). The mixture was stirred at r.t. for 1 h. LC-MS showed 7 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 21 monomer (10.5 g, 14.5 mmol, 75.9%) as a white solid. ESI-LCMS: m/z 776.3 [M+H]⁺; ¹H-NMR (400 MHz, DMSO-d₆): δ 11.40 (d, J=12.2 Hz, 1H), 8.04-7.96 (dd, 1H), 7.43-7.24 (m, 9H), 6.92-6.87 (m, 4H), 5.84 (m, 1H), 4.93 (m, 1H), 4.13 (m, 1H), 3.91-3.39 (m, 17H), 2.82 (t, J=5.6 Hz, 1H), 2.68 (t, J=6.0 Hz, 1H), 1.22-0.97 (m, 12H); ³¹P-NMR (162 MHz, DMSO-d₆): δ 165.06, 157.59.

### Example 22. Synthesis of 5′ End Cap Monomer

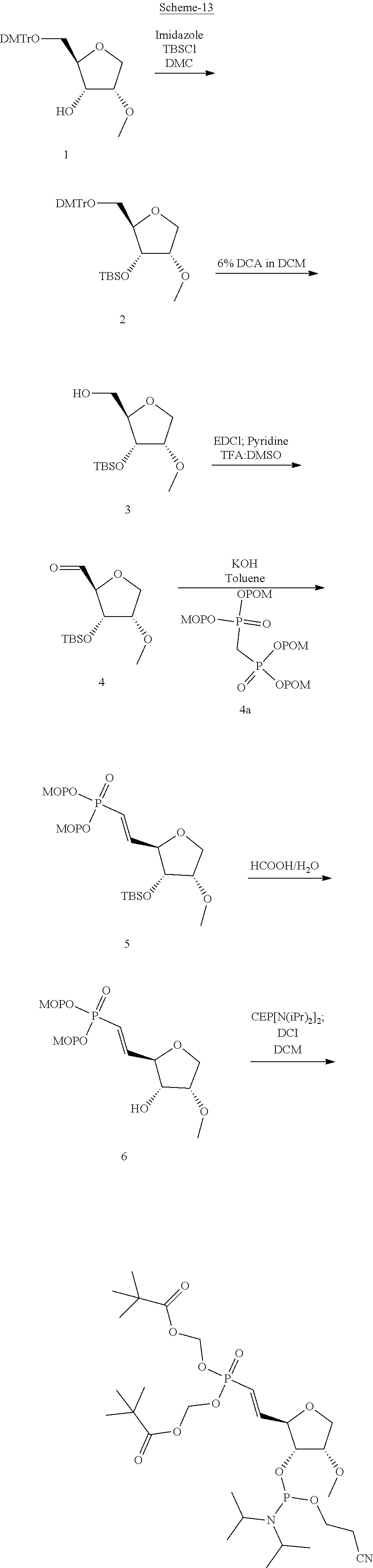

Preparation of (2): To a solution of 1 (11.2 g, 24.7 mmol) in DCM (120 mL), imidazole (4.2 g, 61.9 mmol) and TBSCl (5.6 g, 37.1 mmol) were added at r.t., mixture was stirred at r.t. for 15 hrs, LCMS showed 1 was consumed completely. Mixture was added water (500 mL) and extracted with DCM (50 mL*2). The organic phase was dried over Na₂SO₄ and concentrated to give 2 (16.0 g) as an oil for the next step.

Preparation of (3): To a solution of 2 (16.0 g, 28.4 mmol) was added 6% DCA in DCM (160 mL) and triethylsilane (40 mL) at r.t. The reaction mixture was stirred at r.t. for 2 hrs. TLC showed 2 was consumed completely. Water (300 mL) was added, mixture was extracted with DCM (50 mL*4), organic phase was dried by Na₂SO₄, concentrated by reduce pressure to give crude which was purified by column chromatography (SiO₂, PE/EA=10:1 to 1:1) to give 3 (4.9 g, 65.9% yield) as an oil. ESI-LCMS: m/z 263 [M+H]⁺; ¹H-NMR (400 MHz, DMSO-d₆) δ 4.84-4.50 (m, 1H), 4.3-4.09 (m, 1H), 3.90-3.80 (m, 1H), 3.75-3.67 (m, 1H), 3.65-3.57 (m, 2H), 3.50-3.44 (m, 1H), 3.37-3.28 (m, 4H), 0.95-0.78 (s, 9H), 0.13-0.03 (s, 6H).

Preparation of (4): To a solution of 3 (3.3 g, 12.6 mmol) in DMSO (33 mL) was added EDCI (7.2 g, 37.7 mmol). The mixture was added pyridine (1.1 g, 13.8 mmol) and TFA (788.6 mg, 6.9 mmol). The reaction mixture was stirred at r.t. for 3 hrs. TLC (PE/EA=4:1) showed 3 was consumed. The mixture was diluted with EA and water was added. The product was extracted with EA. The organic layer was washed with brine and dried over Na₂SO₄ and concentrated to give the crude. This resulted in to give 4 (3.23 g) as an oil for the next step.

Preparation of (5): To a solution of 4 (3.3 g, 12.6 mmol) in toluene (30 mL) was added POM ester 4a (reference for 4a Journal of Medicinal Chemistry, 2018, 61 (3), 734-744) (7.9 g, 12.6 mmol) and KOH (1.3 g, 22.6 mmol) at r.t. The reaction mixture was stirred at 40° C. for 8 hrs. LCMS showed 4 was consumed. The mixture was diluted with water and EA was added. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=91/9 Detector, UV 254 nm. This resulted in to give 5 (5.4 g, 9.5 mmol, 75.9% yield) as an oil. ESI-LCMS: m/z 567.2 [M+H]⁺; ¹H-NMR (400 MHz, CDCl₃) δ 6.89-6.77 (m, 1H), 6.07-5.96 (m, 1H), 5.86-5.55 (m, 4H), 4.85-4.73 (m, 1H), 4.36-4.27 (m, 1H), 4.05-3.96 (m, 1H), 3.95-3.85 (m, 1H), 3.73-3.65 (m, 1H), 3.44-3.35 (m, 3H), 1.30-1.25 (s, 18H), 0.94-0.84 (s, 9H), 0.14-0.05 (s, 6H). ³¹P-NMR (162 MHz, CDCl₃) δ 18.30, 15.11.

Preparation of (6): To a solution of 5 (5.4 g, 9.5 mmol) in HCOOH (30 mL)/H₂O (30 mL)=1:1 at r.t. The reaction mixture was stirred at r.t. for 15 hrs. LCMS showed the reaction was consumed. The mixture was diluted with con. NH₄OH till pH=7.5. The product was extracted with EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% HCOOH)=30/70 increasing to CH₃CN/H₂O (0.5% HCOOH)=70/30 within 45 min, the eluted product was collected at CH₃CN/H₂O (0.5% HCOOH)=59/41 Detector, UV 220 nm. This resulted in to give 6 (2.4 g, 5.7 mmol, 59.4% yield) as an oil. ESI-LCMS: m/z 453.2 [M+H]⁺; ¹H-NMR (400 MHz, DMSO-d₆) δ 6.84-6.68 (m, 1H), 6.07-5.90 (m, 1H), 5.64-5.55 (m, 4H), 5.32-5.24 (m, 1H), 4.23-4.15 (m, 1H), 4.00-3.90 (m, 1H), 3.89-3.80 (m, 1H), 3.78-3.69 (m, 2H), 3.37-3.30 (s, 3H), 1.30-1.10 (s, 18H). ³¹P-NMR (162 MHz, DMSO-d₆) δ 18.14.

Preparation of Example 22 monomer: To a solution of 6 (2.1 g, 4.5 mmol) in DCM (21 mL) were added DCI (452.5 mg, 3.8 mmol) and CEP[N(iPr)₂]₂ (1.8 g, 5.9 mmol) at r.t. The reaction mixture was stirred at r.t. for 15 hrs under N₂ atmosphere. LCMS showed 6 was consumed. The mixture was diluted with water. The product was extracted with DCM (30 mL). The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 28 min, the eluted product was collected at CH₃CN/H₂O (0.5% NH₄HCO₃)=80/20 Detector, UV 254 nm. This resulted in to give

Example 22 monomer (2.8 g, 4.3 mmol, 95.2% yield) as an oil. ESI-LCMS: m/z 653.2 [M+H]⁺; ¹H-NMR (400 MHz, DMSO-d₆) δ 6.89-6.77 (m, 1H), 6.11-5.96 (m, 1H), 5.65-5.50 (m, 4H), 4.39-4.34 (d, J=20 Hz, 1H), 4.18-3.95 (m, 2H), 3.94-3.48 (s, 6H), 3.40-3.28 (m, 4H), 2.84-2.75 (m, 2H), 1.26-1.98 (s, 30H). ³¹P-NMR (162 MHz, DMSO-d₆) δ 149.018, 148.736, 17.775, 17.508.

### Example 23. Synthesis of 5′ End Cap Monomer

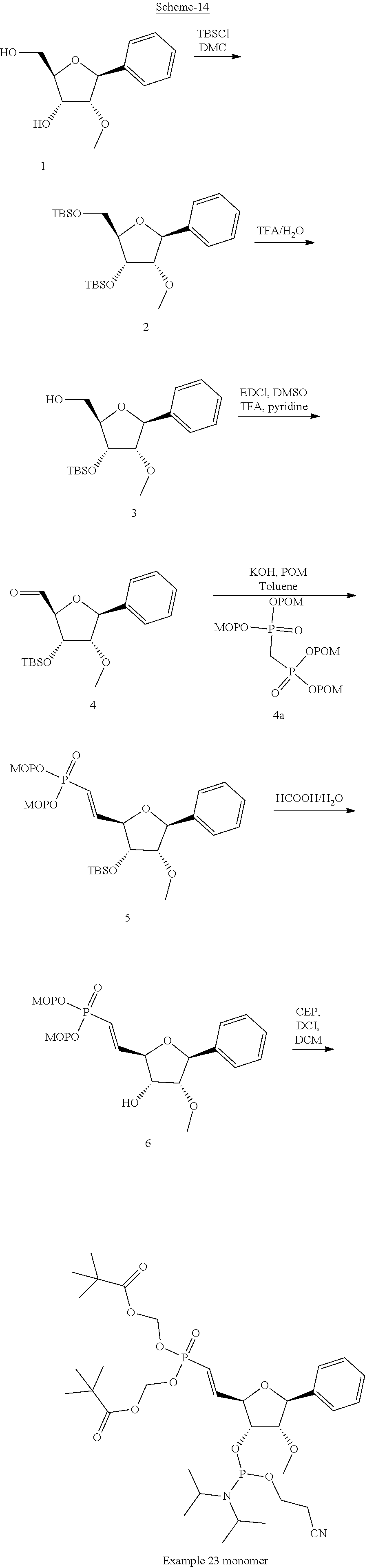

Preparation of (2): To a solution of 1 (ref for 1 Tetrahedron, 2013, 69, 600-606) (10.60 g, 47.32 mmol) in DMF (106 mL), imidazole (11.26 g, 165.59 mmol) and TBSCl (19.88 g, 132.53 mmol) were added. The mixture was stirred at r.t. for 3.5 hrs, LCMS showed 1 was consumed completely. Water was added and extracted with EA, dried over by Na₂SO₄. The filtrate was evaporated under reduced pressure to give 2 (20.80 g, 45.94 mmol, 97.19% yield) for the next step.

Preparation of (3): To a solution of 2 (20.80 g, 45.94 mmol) in THF (248 mL), was added TFA (124 mL) and H₂O (124 mL) at 0° C., reaction mixture was stirred for 30 min. LCMS showed 2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 3 (10.00 g, 29.59 mmol, 64.31% yield). ¹H-NMR (400 MHz, DMSO-d₆): δ 7.33-7.18 (m, 5H), 4.83-4.80 (m, 1H), 4.61-4.59 (m, 1H), 4.21-4.19 (m, 1H), 3.75-3.74 (m, 1H), 3.23 (m, 3H), 3.13 (m, 3H), 2.41-2.40 (m, 1H), 0.81 (m, 9H), 0.00 (m, 6H).

Preparation of (4): To a solution of 3 (3.70 g, 10.95 mmol) in DMSO (37 mL) was added EDCI (6.30 g, 32.84 mmol). Then pyridine (0.95 g, 12.05 mmol) and TFA (0.69 g, 6.02 mmol) was added in N₂ atmosphere. The mixture was stirred for 3 hrs at r.t. LCMS showed 3 was consumed completely. Water was poured into and extracted with EA, washed with sat. NaCl (aq.), dried over by Na₂SO₄. The filtrate was evaporated under reduced pressure to give the crude product which was directly used for next step.

Preparation of (5): To a solution of 4 in toluene (100.00 mL), was added 4a (6.93 g, 10.97 mmol) and KOH (1.11 g, 19.78 mmol). It was stirred for 3.5 hrs at 40° C. in N₂ atmosphere. TLC and LCMS showed 4 was consumed completely. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 5 (4.30 g, 6.70 mmol, 61.17% yield). ¹H-NMR (400 MHz, CDCl₃): δ 7.27-7.26 (m, 4H), 7.17 (m, 1H), 6.94-6.82 (m, 1H), 6.13-6.02 (m, 1H), 5.63-5.56 (m, 4H), 4.90-4.89 (m, 1H), 4.45-4.41 (m, 1H), 3.98-3.95 (m, 1H), 3.39-3.29 (m, 4H), 1.90 (m, 1H), 1.12-0.83 (m, 29H), 0.00 (m, 7H); ³¹P-NMR (162 MHz, CDCl₃): δ 18.021, 14.472.

Preparation of (6): To a solution of 5 (4.30 g, 6.70 mmol) in THF (43.00 mL) was added HCOOH (100 mL) and H₂O (100 mL). It was stirred overnight at r.t. LCMS showed 5 was consumed completely. NH₄OH was poured into it and was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.10 g, 3.98 mmol, 59.32% yield). ¹H-NMR (400 MHz, CDCl₃): δ 7.40-7.28 (m, 5H), 7.11-7.00 (m, 1H), 6.19-6.14 (m, 1H), 5.71-5.68 (m, 4H), 4.95-4.94 (m, 1H), 4.48-4.47 (m, 1H), 4.05-4.03 (m, 1H), 3.62-3.61 (m, 1H), 3.46 (m, 3H), 3.00-2.99 (m, 1H), 1.22 (m, 18H); ³¹P-NMR (162 MHz, CDCl₃): δ 18.134.

Preparation of Example 23 monomer: To a solution of 6 (2.10 g, 3.98 mmol) in DCM (21 mL) was added DCI (410 mg, 3.47 mmol). CEP (1.40 g, 4.65 mmol) was added in a N₂ atmosphere. LCMS showed 6 was consumed completely. DCM and H₂O was poured, the organic phase was washed with water and sat. NaCl (aq.), dried over by Na₂SO₄. The filtrate was evaporated under reduced pressure at 40° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 23 monomer (2.10 g, 2.88 mmol). 1H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H); ³¹P-NMR (162 MHz, DMSO-d₆): δ 149.588, 148.920, 17.355, 17.010.

### Example 24. Synthesis of 5′ End Cap Monomer

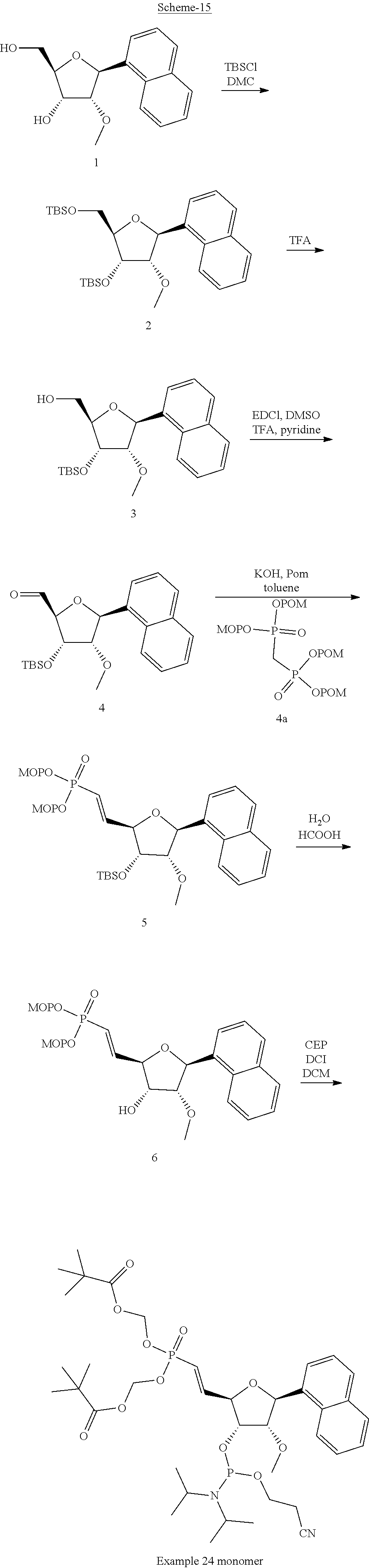

Preparation of (2): To a solution of 1 (5.90 g, 21.50 mmol) in DMF (60.00 mL), imidazole (4.39 g, 64.51 mmol) and TBSCl (7.63 g, 49.56 mmol) were added. The mixture was stirred at r.t. for 3.5 hrs, LCMS showed 1 was consumed completely. Water was added and extracted with EA, dried over by Na₂SO₄. The filtrate was evaporated under reduced pressure to give 2 (11.00 g, 21.91 mmol, 98.19% yield) for the next step. ESI-LCMS: m/z 225.1 [M+H]⁺.

Preparation of (3): To a solution of 2 (11.00 g, 21.91 mmol) in THF (55.00 mL) was added TFA (110.00 mL) and H₂O (55.00 mL) at 0° C., reaction mixture was stirred for 30 min. LCMS showed 2 was consumed completely. Then was extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 3 (6.20 g, 16.32 mmol, 72.94% yield). ESI-LCMS: m/z 411.2 [M+H]⁺.

Preparation of (4): To a solution of 3 (3.50 g, 9.02 mmol) in DMSO (35.00 mL) was added EDCI (5.19 g, 27.06 mmol). Then pyridine (0.78 g, 9.92 mmol) and TFA (0.57 g, 4.96 mmol) was added in N₂ atmosphere. The mixture was stirred for 3 h at r.t. Water was poured into it and was extracted with EA, washed with sat. NaCl (aq.), dried over by Na₂SO₄. The filtrate was evaporated under reduced pressure to give the crude product which was directly used for next step. ESI-LCMS: m/z 406.2 [M+H]⁺.

Preparation of (5): To a solution of 4 in toluene (100.00 mL) was added 4a (5.73 g, 9.07 mmol) and KOH (916.3 g, 16.33 mmol). It was stirred for 3.5 h at 40° C. in N₂ atmosphere. Then was extracted with EA, washed with water and sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 5 (5.02 g, 7.25 mmol, 80.44% yield). ESI-LCMS: m/z 693.2 [M+H]⁺; ³¹P-NMR (162 MHz, DMSO-d₆): δ 17.811

Preparation of (6): To a solution of 5 (4.59 g, 6.63 mmol) in THF (46.00 mL) was added HCOOH (92.00 mL) and H₂O (92.00 mL). It was stirred overnight at r.t. NH₄OH was poured into it and extracted with EA, washed with sat. NaCl (aq.), dried over by Na₂SO₄. The filtrate was evaporated under reduced pressure to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.52 g, 4.36 mmol, 65.80% yield).

Preparation of Example 24 monomer: To a solution of 6 (2.00 g, 3.46 mmol) in DCM (21.00 mL) was added DCI (370.00 mg, 3.11 mmol) and CEP (1.12 g, 4.15 mmol) was added in N₂ atmosphere. DCM and H₂O was poured, the organic phase was washed with water and sat. NaCl (aq.), dried over by Na₂SO₄. The filtrate was evaporated under reduced pressure at 38° C. to give the crude product which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 24 monomer (2.10 g, 2.70 mmol, 78.07% yield). ¹H-NMR (400 MHz, DMSO-d₆): δ 7.39-7.32 (m, 6H), 6.21-6.11 (m, 1H), 5.64-5.61 (m, 4H), 4.91-4.85 (m, 1H), 4.59 (m, 1H), 4.28-4.25 (m, 1H), 3.84-3.60 (m, 5H), 3.36-3.36 (m, 2H), 2.83-2.79 (m, 2H), 1.18-1.14 (m, 29H). ³¹P-NMR (162 MHz, DMSO-d₆): δ 149.588, 148.920, 17.355, 17.010.

### Example 25. Synthesis of Monomer

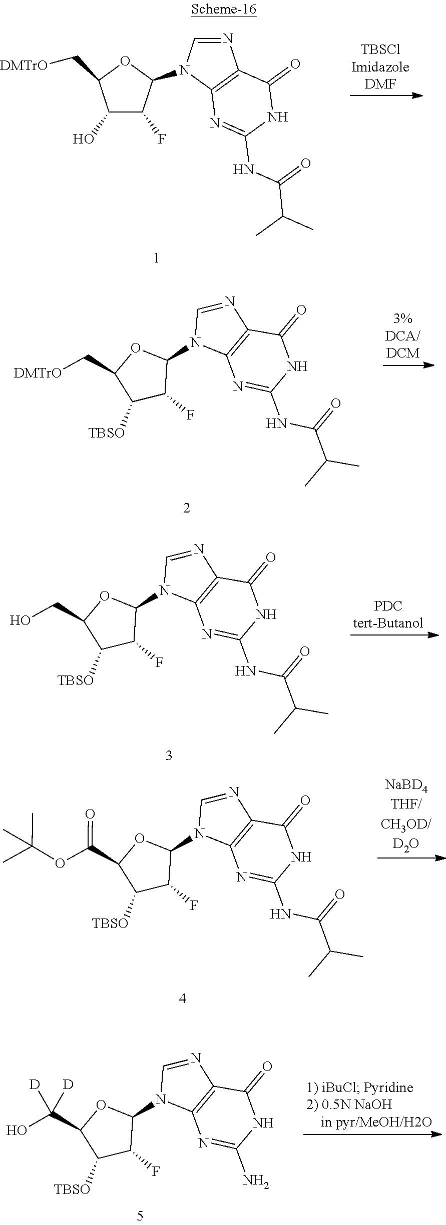

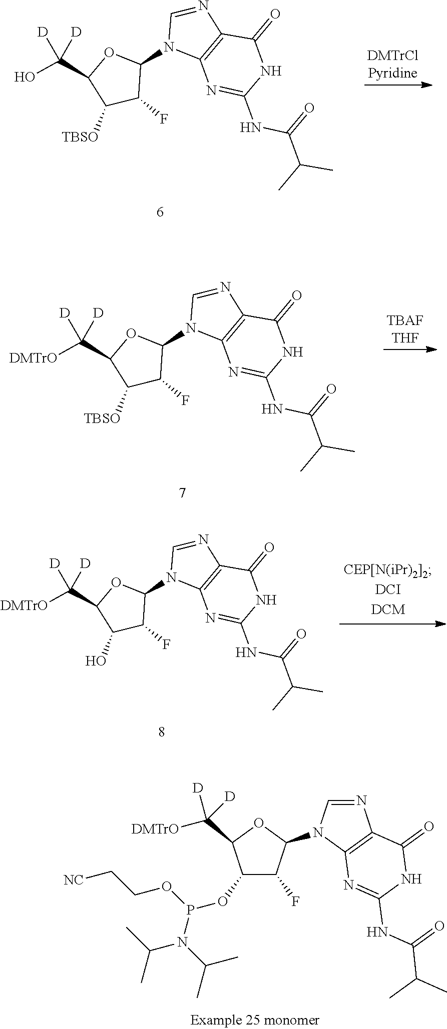

Preparation of (2): To a solution of 1 (35.0 g, 53.2 mmol) in DMF (350 mL) was added imidazole (9.0 g, 133.0 mmol) then added TBSCl (12.0 g, 79.8 mmol) at 0° C. The mixture was stirred at r.t. for 14 hrs. TLC showed 1 was consumed completely. Water was added to the reaction. The product was extracted with EA, The organic layer was washed with NaHCO₃ and brine. Then the solution was concentrated under reduced pressure the crude 2 (41.6 g) as a white solid which was used directly for next step. ESI-LCMS: m/z 772 [M+H]⁺.

Preparation of (3): To a solution of 2 (41.0 g, 53.1 mmol) in 3% DCA (53.1 mmol, 350 mL) and Et₃SiH (53.1 mmol, 100 mL) at 0° C. The mixture was stirred at 0° C. for 0.5 h. TLC showed 2 was consumed completely. NaHCO₃ was added to the reaction. The product was extracted with EA, The organic layer was washed with NaHCO₃ and brine. Then the solution was concentrated under reduced pressure. The residue silica gel column chromatography (eluent, DCM/MeOH=100:1˜20:1). This resulted in to give 3 (20.0 g, 41.7 mmol, 78.6% over two step) as a white solid. ESI-LCMS: m/z 470 [M+H]⁺; ¹H-NMR (400 MHz, DMSO-d₆): δ 12.12 (s, 1H), 11.67 (s, 1H), 8.28 (s, 1H), 6.12-6.07 (dd, J=15 Hz, 1H), 5.75 (d, J=5 Hz, 1H), 5.48-5.24 (m, 2H), 4.55-4.49 (m, 1H), 3.97 (s, 1H), 3.75-3.55 (m, 2H), 2.79-2.76 (m, 1H), 1.12 (d, J=6 Hz, 6H), 0.88 (s, 9H), 0.11 (d, J=6 Hz, 6H).

Preparation of (4): To the solution of 3 (20 g, 42.6 mmol) in dry DCM (100 mL) and DMF (60 mL) was added PDC (20. g, 85.1 mmol), tert-butyl alcohol (63.1 g, 851.8 mmol) and Ac₂O (43.4 g, 425.9 mmol) at r.t. under N₂ atmosphere. And the reaction mixture was stirred at r.t. for 2 h. The solvent was removed to give a residue which was purified by silica gel column chromatography (eluent, PE:EA=4:1˜2:1) to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 4 (16.0 g, 29.0 mmol, 68.2% yield) as a white solid. ESI-LCMS: m/z 540 [M+H]⁺; ¹H-NMR (400 MHz, DMSO-d₆): δ 12.12 (s, 1H), 11.69 (s, 1H), 8.28 (s, 1H), 6.21-6.17 (dd, J=15 Hz, 1H), 5.63-5.55 (m, 1H), 4.75-4.72 (m, 1H), 4.41 (d, J=5 Hz, 1H), 2.79-2.76 (m, 1H), 1.46 (s, 9H), 1.13-1.11 (m, 6H), 0.90 (s, 9H), 0.14 (d, J=2 Hz, 6H).

Preparation of (5): To the solution of 4 (16.0 g, 29.6 mmol) in dry THF/MeOD/D₂O=10/2/1 (195 mL) was added NaBD₄ (3.4 g, 88.9 mmol) at r.t. and the reaction mixture was stirred at 50° C. for 2 h. After completion of reaction, adjusted pH value to 7 with CH₃COOD, after addition of water, the resulting mixture was extracted with EA (300 mL). The combined organic layer was washed with water and brine, dried over Na₂SO₄, Then the solution was concentrated under reduced pressure the crude 5 (11.8 g) as a white solid which was used directly for next step. ESI-LCMS: m/z 402 [M+H]⁺.

Preparation of (6): To a solution of 5 (5.0 g, 12.4 mmol) in pyridine (50 mL) was added iBuCl (2.6 g, 24.9 mmol) at 0° C. under N₂ atmosphere. The mixture was stirred at r.t. for 14 h. TLC showed 5 was consumed completely. Then the solution diluted with EA. The organic layer was washed with NaHCO₃ and brine. Then the solution was concentrated under reduced pressure to give the crude. To a solution of the crude in pyridine (50 mL) was added 2N NaOH (MeOH/H₂O=4:1, 15 mL) at 0° C. The mixture was stirred at 0° C. for 10 min. Then the solution diluted with EA The organic layer was washed with NH₄Cl and brine. Then the solution was concentrated under reduced pressure the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 6 (6 g, 10.86 mmol, 87.17% yield) as a white solid. ESI-LCMS: m/z 472.2 [M+H]⁺; ¹H-NMR (400 MHz, DMSO-d₆): δ 12.12 (s, 1H), 11.67 (s, 1H), 8.28 (s, 1H), 6.12-6.07 (dd, J=15 Hz, 1H), 5.48-5.24 (m, 2H), 5.22 (s, 1H), 4.55-4.49 (m, 1H), 3.97 (d, J=5 Hz, 1H), 2.79-2.76 (m, 1H), 1.12 (d, J=6 Hz, 6H), 0.88 (s, 9H), 0.11 (d, J=6 Hz, 6H).

Preparation of (7): To a solution of 6 (3.8 g, 8.1 mmol) in pyridine (40 mL) was added DMTrCl (4.1 g, 12.1 mmol) at 20° C. The mixture was stirred at 20° C. for 1 h. TLC showed 7 was consumed completely. Water was added to the reaction. The product was extracted with EA, The organic layer was washed with NaHCO₃ and brine. Then the solution was concentrated under reduced pressure to give the crude product of 7 (6 g, 7.6 mmol, 94.3% yield) as a yellow solid. ESI-LCMS: m/z 775 [M+H]⁺.

Preparation of (8): To a solution of 7 (6.0 g, 7.75 mmol) in THF (60 mL) was added TBAF (2.4 g, 9.3 mmol). The mixture was stirred at r.t. for 1 h. TLC showed 7 was consumed completely. Water was added to the reaction. The product was extracted with EA, The organic layer was washed with NaHCO₃ and brine. Then the solution was concentrated under reduced pressure, the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 8 (4.0 g, 5.9 mmol, 76.6% yield) as a white solid. ESI-LCMS: m/z 660 [M+H]⁺; ¹H-NMR (400 MHz, DMSO-d₆): δ 12.12 (s, 1H), 11.67 (s, 1H), 8.12 (s, 1H), 7.34-7.17 (m, 9H), 6.83-6.78 (m, 4H), 6.23-6.18 (m, 1H), 5.66 (d, J=7 Hz, 1H), 5.48-5.35 (m, 1H), 4.65-4.54 (m, 1H), 3.72 (d, J=2 Hz, 6H), 2.79-2.73 (m, 1H), 1.19-1.06 (m, 6H).

Preparation of Example 25 monomer: To a solution of 9 (4.0 g, 6.1 mmol) in DCM (40 mL) was added DCI (608 mg, 5.1 mmol) and CEP (2.2 g, 7.3 mmol) under N₂ pro. The mixture was stirred at 20° C. for 0.5 h. TLC showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 25 monomer (5.1 g, 5.81 mmol, 95.8% yield) as a white solid. ESI-LCMS: m/z 860 [M+H]⁺; ¹H-NMR (400 MHz, DMSO-d₆): δ 12.12 (s, 1H), 11.67 (s, 1H), 8.12 (s, 1H), 7.34-7.17 (m, 9H), 6.83-6.78 (m, 4H), 6.23-6.18 (m, 1H), 5.67-5.54 (m, 1H), 4.70-4.67 (m, 1H), 4.23-4.20 (m, 1H), 3.72 (m, 6H), 3.60-3.48 (m, 3H), 2.79-2.58 (m, 3H), 1.13-0.94 (m, 18H); ³¹P-NMR (162 MHz, DMSO-d₆): δ 150.31, 150.26, 140.62, 149.57.

### Example 26: Synthesis of Monomer

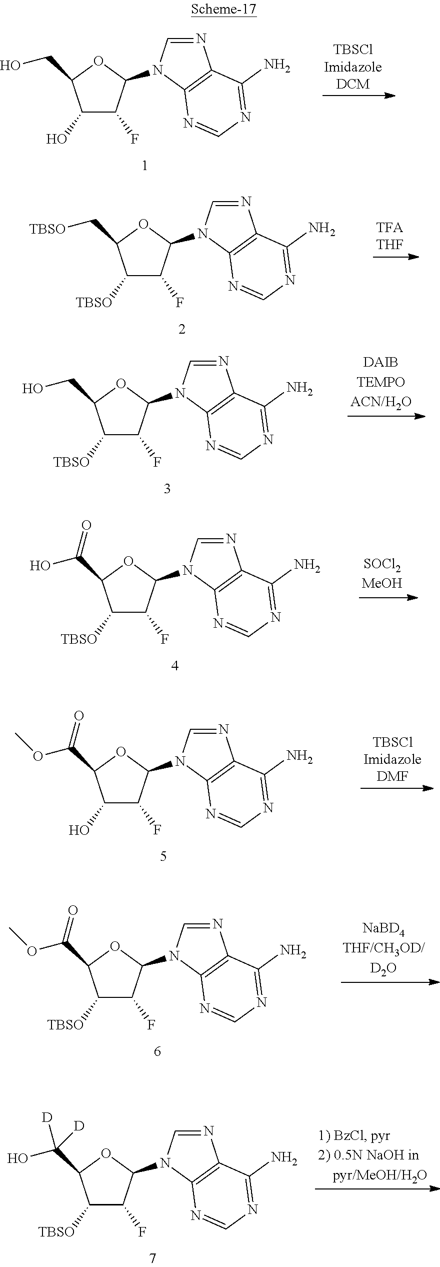

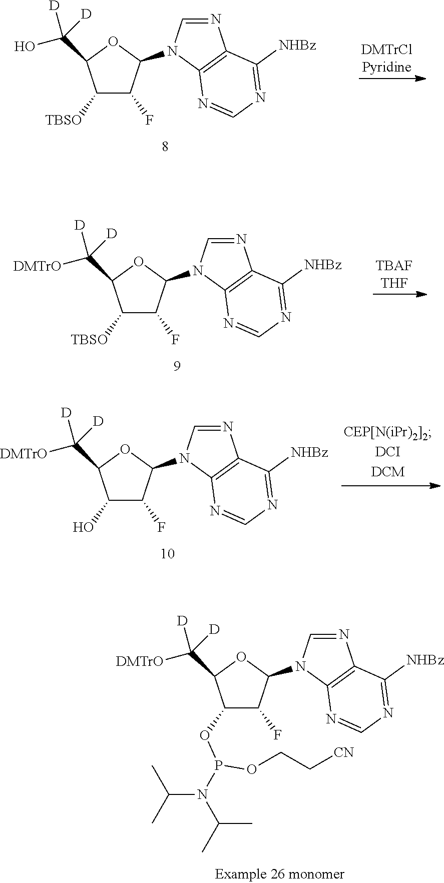

Preparation of (2): To a solution of 1 (35 g, 130.2 mmol) in DMF (350 mL) was added imidazole (26.5 g, 390.0 mmol) then added TBSCl (48.7 g, 325.8 mmol) at 0° C. The mixture was stirred at r.t. for 14 h. TLC showed 1 was consumed completely. Water was added to the reaction. The product was extracted with EA, The organic layer was washed with NaHCO₃ and brine. Then the solution was concentrated under reduced pressure the crude 2 (64.6 g) as a white solid which was used directly for next step. ESI-LCMS: m/z 498 [M+H]⁺.

Preparation of (3): To a solution of 2 (64.6 g, 130.2 mmol) in THF (300 mL) and added TFA/H₂O (1:1, 300 mL) at 0° C. The mixture was stirred at 0° C. for 2 h. TLC showed 2 was consumed completely. NaHCO₃ was added to the reaction. The product was extracted with EA, The organic layer was washed with NaHCO₃ and brine. Then the solution was concentrated under reduced pressure. The residue was purified by silica gel column chromatography (eluent, DCM:MEOH=100:1˜20:1). This resulted in to give 3 (31.3 g, 81.7 mmol, 62.6% over two step) as a white solid. ESI-LCMS: m/z 384 [M+H]⁺.

Preparation of (4): To a solution of 3 (31.3 g, 81.7 mmol) in ACN/H₂O (1:1, 350 mL) was added DAIB (78.0 g, 244.0 mmol) and Tempo (3.8 g, 24.4 mmol). The mixture was stirred at 40° C. for 2 h. TLC showed 3 was consumed completely. Then filtered to give 4 (22.5 g, 55.5 mmol, 70.9%) as a white solid. ESI-LCMS: m/z 398 [M+H]⁺.

Preparation of (5): To a solution of 4 (22.5 g, 55.5 mmol) in MeOH (225 mL) held at −15° C. with an ice/MeOH bath was added SOCl₂ (7.6 mL, 94.5 mmol), dropwise at such a rate that the reaction temp did not exceed 7° C. After the addition was complete, cooling was removed, the reaction was allowed to stir at room temp. The mixture was stirred at r.t. for 14 h. TLC showed 4 was consumed completely. Then the solution was concentrated under reduced pressure to get crude 5 (23.0 g) as a white solid which was used directly for next step. ESI-LCMS: m/z 298 [M+H]⁺.

Preparation of (6): To a solution of 5 (23 g, 55.5 mmol) in DMF (220 mL) was added imidazole (11.6 g, 165.0 mmol) then added TBSCl (12.3 g, 82.3 mmol) at 0° C. The mixture was stirred at 20° C. for 14 h. TLC showed 1 was consumed completely. Water was added to the reaction. The product was extracted with EA, The organic layer was washed with NaHCO₃ and brine. Then the solution was concentrated under reduced pressure. The residue was purified by silica gel column chromatography (eluent, DCM:MEOH=100:1˜20:1). This resulted in to give 6 (21.3 g, 51.1 mmol, 90% over two step) as a white solid. ESI-LCMS: m/z 412 [M+H]⁺.

Preparation of (7): To the solution of 6 (21.0 g, 51.0 mmol) in dry THF/MeOD/D₂O=10/2/1 (260.5 mL) was added NaBD₄ (6.4 g, 153.1 mmol) at r.t. and the reaction mixture was stirred at 50° C. for 2 h. After completion of reaction, the resulting mixture was added CH₃COOD to pH=7, after addition of water, the resulting mixture was extracted with EA (300 mL). The combined organic layer was washed with water and brine, dried over Na₂SO₄. Then the solution was concentrated under reduced pressure and the residue was used for next step without further purification. ESI-LCMS: m/z 386 [M+H]⁺.

Preparation of (8): To a stirred solution of 7 (14.0 g, 35 mmol) in pyridine (50 mL) were added BzCl (17.2 g, 122.5 mmol) at 0° C. under N₂ atmosphere. The mixture was stirred at r.t. for 14 h. TLC showed 7 was consumed completely. Then the solution diluted with EA. The organic layer was washed with NaHCO₃ and brine. Then the solution was concentrated under reduced pressure and the residue was used for next step without further purification. To a solution of the crude in pyridine (300 mL) then added 2M NaOH (MeOH: H₂O=4:1, 60 mL) at 0° C. The mixture was stirred at 0° C. for 10 min. Then the solution diluted with EA. The organic layer was washed with NH₄Cl and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 8 (14 g, 28.02 mmol, 69.21% yield) as a white solid. ESI-LCMS: m/z 490 [M+H]⁺; ¹H-NMR (400 MHz, DMSO-d₆): δ 11.24 (s, 1H), 8.76 (s, 1H), 8.71 (m, 1H), 8.04 (d, J=7 Hz, 2H), 7.66-7.10 (m, 5H), 6.40-6.35 (dd, 1H), 5.71-5.56 (m, 1H), 5.16 (s, 1H), 4.79-4.72 (m, 1H), 4.01 (m, 1H), 0.91 (s, 9H), 0.14 (m, 6H).

Preparation of (9): To a solution of 8 (5.1 g, 10.4 mmol) in pyridine (50 mL) was added DMTrCl (5.3 g, 15.6 mmol). The mixture was stirred at r.t. for 1 h. TLC showed 8 was consumed completely. Water was added to the reaction. The product was extracted with EA, The organic layer was washed with NaHCO₃ and brine. Then the solution was concentrated under reduced pressure and the residue was used for next step without further purification. ESI-LCMS: m/z 792 [M+H]⁺.

Preparation of (10): To a solution of 9 (7.9 g, 10.0 mmol) in THF (80 mL) was added 1M TBAF in THF (12 mL). The mixture was stirred at r.t. for 1 h. TLC showed 9 was consumed completely. Water was added to the reaction. The product was extracted with EA, The organic layer was washed with NaHCO₃ and brine. Then the solution was concentrated under reduced pressure the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 10 as a white solid. ESI-LCMS: m/z 678 [M+H]⁺; ¹H-NMR (400 MHz, DMSO-d₆): δ 11.25 (s, 1H), 8.74 (s, 1H), 8.62 (s, 1H), 8.04 (d, J=7 Hz, 2H), 7.66-7.53 (m, 3H), 7.33-7.15 (m, 9H), 6.82-6.78 (m, 4H), 6.43 (d, J=20 Hz, 1H), 5.76-5.60 (m, 1H), 4.88-4.80 (m, 1H), 4.13 (d, J=8 Hz, 1H), 3.71 (m, 6H).

Preparation of Example 26 monomer: To a solution of 10 (6.2 g, 9.1 mmol) in DCM (60 mL) was added DCI (1.1 g, 9.4 mmol) and CEP (3.3 g, 10.9 mmol) under N₂ pro. The mixture was stirred at 20° C. for 0.5 h. TLC showed 10 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 26 monomer (7.5 g, 8.3 mmol, 90.7%) as a white solid. ESI-LCMS: m/z 878 [M+H]⁺; ¹H-NMR (400 MHz, DMSO-d₆): δ 11.25 (s, 1H), 8.68-8.65 (dd, 2H), 8.04 (m, 2H), 7.66-7.53 (m, 3H), 7.33-7.15 (m, 9H), 6.82-6.78 (m, 4H), 6.53-6.43 (m, 1H), 5.96-5.81 (m, 1H), 5.36-5.15 (m, 1H), 4.21 (m, 1H), 3.86-3.52 (m, 10H), 2.79-2.61 (m, 2H), 1.21-0.99 (m, 12H); ³¹P-NMR (162 MHz, DMSO-d₆): δ 149.60, 149.56, 149.48.

### Example 27. Synthesis of End Cap Monomer

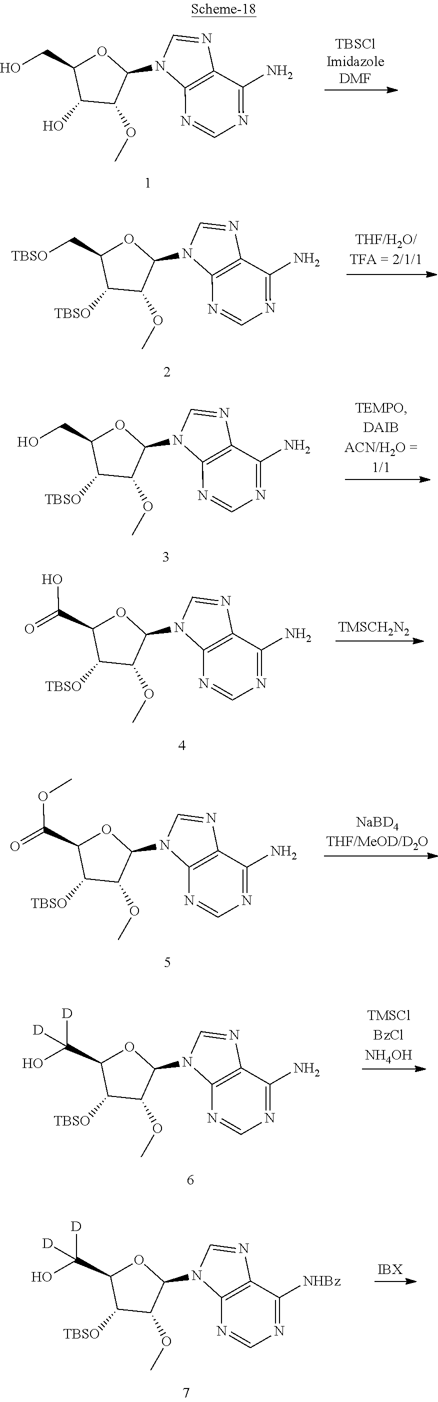

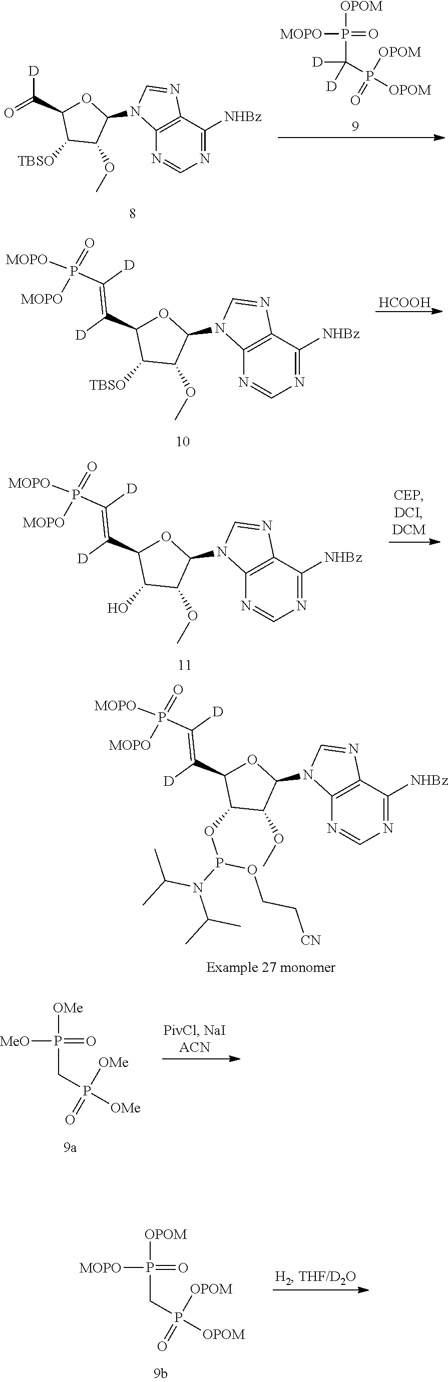

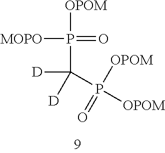

Preparation of (2): To a solution of 1 (20.0 g, 71.2 mmol) in dry pyridine (200.0 mL) was added TBSCl (26.8 g, 177.9 mmol) and imidazole (15.6 g, 227.8 mmol). The mixture was stirred at r.t. for 15 h. TLC showed 1 was consumed completely. The reaction mixture was concentrated to give residue. The residue was quenched with DCM (300.0 mL). The DCM layer was washed with H₂O (100.0 mL*2) and brine. The DCM layer concentrated to give crude 2 (45.8 g) as a yellow oil. The crude used to next step directly. ESI-LCMS m/z 510.5 [M+H]⁺.

Preparation of (3): To a mixture solution of 2 (45.8 g) in THF (300.0 mL) was added mixture of H₂O (100.0 mL) and TFA (100.0 mL) at 0° C. over 30 min. Then the reaction mixture was stirred at 0° C. for 4 h. TLC showed the 2 was consumed completely. The reaction mixture pH was adjusted to 7-8 with NH₃·H₂O (100 mL). Then the mixture was extracted with EA (500.0 mL*2). The combined EA layer was washed with brine and concentrated to give crude which was purified by c.c. (PE:EA=5:1 ˜1:0) to give compound 3 (21.0 g, 53.2 mmol, 74.7% yield over 2 steps) as a white solid. ESI-LCMS m/z 396.2 [M+H]⁺.

Preparation of (4): To a solution of 3 (21.0 g, 53.2 mmol) in ACN (100.0 mL) and water (100.0 mL) were added (diacetoxyiodo)benzene (51.0 g, 159.5 mmol) and TEMPO (2.5 g, 15.9 mmol), The reaction mixture was stirred at 40° C. for 1 h. TLC showed the 3 was consumed completely. The reaction mixture was cooled down to r.t. and filtered, the filtrate was concentrated to give crude which was purified by crystallization (ACN) to give 4 (14.5 g, 35.4 mmol, 66.2% yield). ESI-LCMS m/z 410.1 [M+H]⁺.

Preparation of (5): To a solution of 4 (14.5 g, 35.4 mmol) in toluene (90.0 mL) and MeOH (60.0 mL) was added trimethylsilyldiazomethane (62.5 mL, 2.0 M, 141.8 mmol) at 0° C., then stirred at r.t. for 2 h. TLC showed the 4 was consumed completely. The solvent was removed under reduce pressure, the residue was purified by crystallization (ACN) to give 5 (10.0 g, 23.6 mmol, 66.6% yield). ESI-LCMS m/z 424.2 [M+H]⁺

Preparation of (6): To the solution of 5 (10.0 g, 23.6 mmol) in dry THF/MeOD/D₂O=10/2/1 (100.0 mL) was added NaBD₄ (2.98 g, 70.9 mmol) three times during an hour at 40° C., the reaction mixture was stirred at r.t. for 2.0 h. The resulting mixture was added CH₃COOD change pH=7.5, after addition of water, the resulting mixture was extracted with EA (50.0 mL*3). The combined organic layer was washed with water and brine, dried over Na₂SO₄, concentrated to give a residue which was purified by c.c. (PE/EA=1:1 ˜1:0). This resulted in to give 6 (6.1 g, 15.4 mmol, 65.3% yield) as a white solid. ESI-LCMS m/z 398.1 [M+H]⁺; ¹H-NMR (400 MHz, DMSO-d₆) δ 8.28 (s, 1H), 8.02 (s, 1H), 7.23 (s, 2H), 5.86 (d, J=6.4 Hz, 1H), 5.26 (s, 1H), 4.42-4.41 (m, 1H), 4.35-4.32 (m, 1H), 3.82 (d, J=2.6 Hz, 1H), 3.14 (s, 3H), 0.78 (s, 9H), 0.00 (d, J=0.9 Hz, 6H).

Preparation of (7): To a solution of 6 (6.1 g, 15.4 mmol) in pyridine (60.0 mL) was added the benzoyl chloride (6.5 g, 46.2 mmol) drop wise at 5° C. The reaction mixture was stirred at r.t. for 2 h. TLC showed the 6 was consumed completely. The reaction mixture was cooled down to 10° C. and quenched with H₂O (20.0 mL), extracted with EA (200.0 mL*2), combined the EA layer. The organic phase was washed with brine and dried over Na₂SO₄, concentrated to give the crude (12.0 g) which was dissolved in pyridine (60.0 mL), cooled to 0° C., 20.0 mL NaOH (2 M in methanol: H₂O =4:1) was added and stirred for 10 min. The reaction was quenched by saturated solution of ammonium chloride, the aqueous layer was extracted with EA (200.0 mL*2), combined the EA layer, washed with brine and dried over Na₂SO₄, concentrated. The residue was purified by c.c. (PE/EA=10:1 ˜1:1) to give 7 (7.0 g, 13.9 mmol, 90.2% yield). ESI-LCMS m/z 502.2 [M+H]⁺; ¹H-NMR (400 MHz, DMSO-d₆) δ 11.24 (s, 1H, exchanged with D₂O) 8.77 (s, 2H), 8.04-8.06 (m, 2H), 7.64-7.66 (m, 2H), 7.54-7.58 (m, 2H), 6.14-6.16 (d, J=5.9 Hz, 1H), 5.20-5.23 (m, 1H), 4.58-4.60 (m, 1H), 4.52-4.55 (m, 1H), 3.99-4.01 (m, 1H), 3.34 (s, 4H), 0.93 (s, 9H), 0.14-0.15 (d, J=1.44 Hz, 6H).

Preparation of (8): To a stirred solution of 7 (5.5 g, 10.9 mmol) in DMSO (55.0 mL) was added EDCI (6.3 g, 32.9 mmol), pyridine (0.9 g, 10.9 mmol) and TFA (0.6 g, 5.5 mmol), the reaction mixture was stirred at r.t. for 15 h. The reaction was quenched with water and extracted with EA (100.0 mL). The organic phase was washed by brine, dried over Na₂SO₄, The organic phase was evaporated to dryness under reduced pressure to give a residue 8 (4.8 g) which was used directly to next step. ESI-LCMS: m/z 517.1 [M+H₂O]⁺.

Preparation of (9b): A solution of 9a (35.0 g, 150.8 mmol) and NaI (90.5 g, 603.4 mmol) in dry ACN (180.0 mL) was added chloromethyl pivalate (113.6 g, 754.3 mmol) at r.t., the reaction was stirred at 80° C. for 4 h. The reaction was cooled to r.t. and quenched by water, then the mixture was extracted with EA (500.0 mL*3), combined the organic layer was washed with saturated solution of ammonium chloride, followed by with brine and dried over Na₂SO₄. Then the organic layer was concentrated to give a residue which was purified by c.c., this resulted in to give 9b (38.0 g, 60.1 mmol, 39.8% yield) as a white solid. ESI-LCMS m/z 655.2 [M+Na]⁺; ¹H-NMR (400 MHz, CDCl₃): δ 5.74-5.67 (m, 8H), 2.67 (t, J=21.6 Hz, 2H), 1.23 (s, 36H).

Preparation of (9): 3.8 g 10% Pd/C was washed with dry THF (30.0 mL) three times. Then transferred into a round-bottom flask charged with 9b (38.0 g, 60.1 mmol) and solvent (dry THF: D₂O=5:1, 400.0 mL), the mixture was stirred at 80° C. under 1 L H₂ balloon for 15 h. The reaction was cooled to r.t. and extracted with EA (500.0 mL*3), combined the organic layer was washed with brine and dried over Na₂SO₄. The residue 9 (3.0 g, 3.7 mmol, 38.8% yield) as a white solid was used directly to next step without further purification. ESI-LCMS m/z 657.2 [M+Na]⁺; ¹H-NMR (400 MHz, CDCl₃): δ 5.74-5.67 (m, 8H), 1.23 (s, 36H).

Preparation of (10): A solution of 8 (4.8 g, 9.6 mmol), 9 (7.3 g, 11.5 mmol) and K₂CO₃ (4.0 g, 38.8 mmol) in dry THF (60.0 mL) and D₂O (20.0 mL) was stirred at r.t. 18 h. LC-MS showed 8 was consumed completely. The product was extracted with EA (300.0 mL) and the organic layer was washed with brine and dried over Na₂SO₄. Then the organic layer was concentrated to give a residue which was purified by c.c. (PE/EA=5:1 ˜1:1) and MPLC. This resulted in to give 10 (3.0 g, 3.7 mmol, 38.8% yield) as a white solid. ESI-LCMS m/z 806.4 [M+H]⁺; ¹H-NMR (400 MHz, DMSO-d₆): δ 11.25 (s, 1H, exchanged with D₂O) 8.75 (s, 2H), 8.07-8.05 (d, J=8.0 Hz, 2H), 7.67-7.54 (m, 3H), 6.05 (d, J=5.1 Hz, 1H), 5.65-5.58 (m, 4H), 4.80-4.70 (m, 2H), 4.59-4.57 (m, 1H), 3.36 (s, 3H), 1.11 (s, 9H), 1.10 (s, 9H), 0.94 (s, 9H), 0.17-0.16 (m, 6H); ³¹P NMR (162 MHz, DMSO-d₆) δ 17.02.

Preparation of (11): To a round-bottom flask was added 10 (3.0 g, 3.7 mmol) in a mixture of H₂O (30.0 mL), HCOOH (30.0 mL). The reaction mixture was stirred at 40° C. for 15 hrs. LC-MS showed the 10 was consumed completely. The reaction mixture was adjusted the pH=6-7 with con. NH₃·H₂O (100.0 mL). Then the mixture was extracted with DCM (100.0 mL*3). The combined DCM layer was dried over Na₂SO₄. Filtered and filtrate was concentrated to give crude which was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 20 min, the eluted product was collected at CH₃CN/H₂O (0.5% NH₄HCO₃)=3/2; Detector, UV 254 nm. To give product 11 (1.8 g, 2.6 mmol, 70.3% yield). ESI-LCMS m/z=692.2 [M+H]⁺; ¹H-NMR (400 MHz, DMSO-d₆): δ 11.11 (s, 1H, exchanged with D₂O) 8.71-8.75 (d, J=14.4, 2H), 8.04-8.06 (m, 2H), 7.64-7.65 (m, 1H), 7.54-7.58 (m, 2H), 6.20-6.22 (d, J=5.4, 2H), 5.74-5.75 (d, J=5.72, 2H), 5.56-5.64 (m, 4H), 4.64-4.67 (m, 1H), 4.58-4.59 (m, 1H), 4.49-4.52 (m, 1H), 3.37 (s, 3H), 1.09-1.10 (d, J=1.96, 18H); ³¹P NMR (162 MHz, DMSO-d₆) δ 17.46.

Preparation of Example 27 monomer: To a solution of 11 (1.8 g, 2.6 mmol) in DCM (18.0 mL) was added the DCI (276.0 mg, 2.3 mmol), then CEP[N(ipr)₂]₂ (939.5 mg, 3.1 mmol) was added. The mixture was stirred at r.t. for 1 h. TLC showed 11 consumed completely. The reaction mixture was washed with H₂O (50.0 mL*2) and brine (50.0 mL*2), dried over Na₂SO₄ and concentrated to give crude which was purified by Flash-Prep-HPLC with the following conditions (IntelFlash-1): Column, C18 silica gel; mobile phase, CH₃CN/H₂O (0.5% NH₄HCO₃)-1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. The product was concentrated to give Example 27 monomer (2.0 g, 2.2 mmol, 86.2% yield) as a white solid. ESI-LCMS m/z 892.3 [M+H]⁺; ¹H-NMR (400 MHz, DMSO-d₆): δ 11.27 (s, 1H, exchanged with D₂O) 8.72-8.75 (m, 2H), 8.04-8.06 (m, 2H), 7.54-7.68 (m, 3H), 6.20-6.26 (m, 1H), 5.57-5.64 (m, 4H), 4.70-4.87 (m, 3H), 3.66-3.88 (m, 4H), 3.37-3.41 (m, 3H), 2.82-2.86 (m, 2H), 1.20-1.21 (m, 12H), 1.08-1.09 (m, 18H); ³¹P-NMR (162 MHz, DMSO-d₆): δ 150.03, 149.19, 17.05, 16.81.

### Example 28. Synthesis of 5′ End Cap Monomer

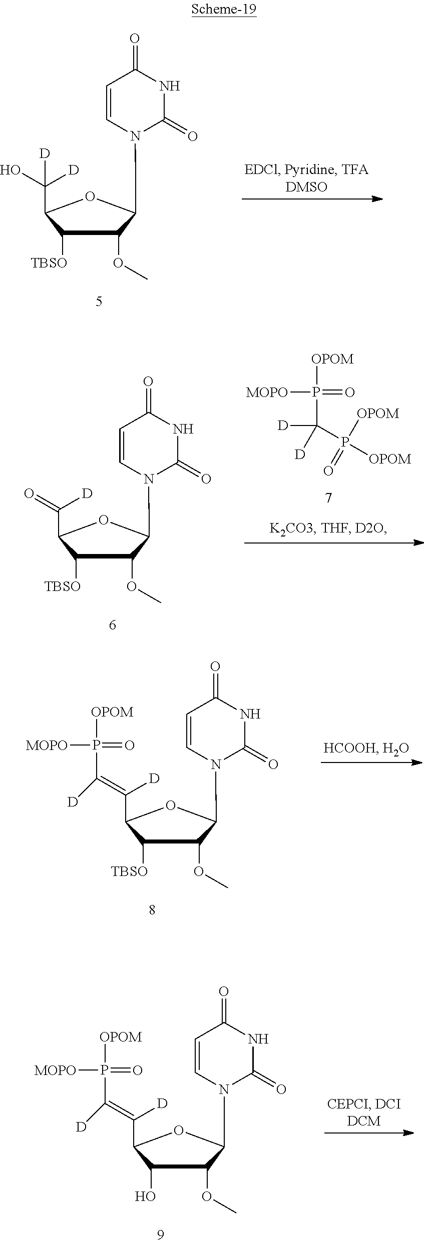

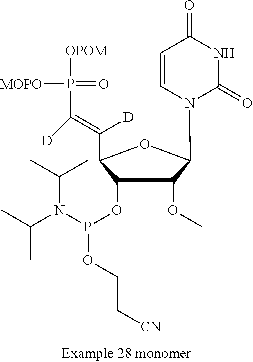

Preparation of (6): To a stirred solution of 5 (8.0 g, 21.3 mmol, Scheme 3) in DMSO (80.0 mL) were added EDCI (12.2 g, 63.9 mmol), pyridine (1.7 g, 21.3 mmol), TFA (1.2 g, 10.6 mmol) at r.t. And the reaction mixture was stirred at r.t. for 1.5 h. The reaction was quenched with water and extracted with EA (200.0 mL). The organic phase was washed by brine, dried over Na₂SO₄, The organic phase was evaporated to dryness under reduced pressure to give a residue 6 which was used directly to next step. ESI-LCMS: m/z 372.3 [M+H]⁺.

Preparation of (8): To a solution of K₂CO₃ (5.5 g, 8.3 mmol) in dry THF (60.0 mL) and D₂O (20.0 mL) was added a solution of 6 (8.0 g, 21.5 mmol) in dry THF (10.0 mL). The reaction mixture was stirred at r.t. overnight. LC-MS showed 6 was consumed completely. The product was extracted with EA (300.0 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 8 (5.0 g, 7.3 mmol, 40.0%) as a white solid. ESI-LCMS: m/z 679.3 [M+H]⁺; ¹H-NMR (400 MHz, Chloroform-d): δ 9.91 (s, 1H), 7.29 (d, J=8.1 Hz, 1H), 5.82 (d, J=2.7 Hz, 1H), 5.72 (d, J=8.1 Hz, 1H), 5.65-5.54 (m, 4H), 4.43 (dd, J=7.2, 3.2 Hz, 1H), 3.92 (dd, J=7.2, 5.0 Hz, 1H), 3.65 (dd, J=5.1, 2.7 Hz, 1H), 3.44 (s, 3H), 1.13 (s, 18H), 0.82 (s, 9H), 0.01 (d, J=4.8 Hz, 6H); ³¹P NMR (162 MHz, Chloroform-d): δ 16.40.

Preparation of (9): To a solution of HCOOH (50.0 mL) and H₂O (50.0 mL) was added 8 (5.0 g, 7.3 mmol). The reaction mixture was stirred at 40° C. overnight. LC-MS showed 8 was consumed completely. A solution of NaHCO₃ (500.0 mL) was added. The product was extracted with EA (300.0 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/2 within 20 min, the eluted product was collected at CH₃CN/H₂O (0.5% NH₄HCO₃)=1/1; Detector, UV 254 nm. This resulted in to give 9 (3.0 g, 5.4 mmol, 73.2%) as a white solid. ESI-LCMS: m/z 565.2 [M+H]⁺; ¹H-NMR (400 MHz, DMSO-d₆): δ 11.43 (s, 1H), 7.64 (d, J=8.1 Hz, 1H), 5.83 (d, J=4.3 Hz, 1H), 5.69-5.56 (m, 5H), 5.54 (d, J=6.7 Hz, 1H), 4.37 (dd, J=6.1, 2.9 Hz, 1H), 4.12 (q, J=6.1 Hz, 1H), 3.96 (dd, J=5.4, 4.3 Hz, 1H), 3.39 (s, 3H), 1.16 (s, 18H); ³¹P NMR (162 MHz, DMSO-d₆): δ 17.16.

Preparation of Example 28 monomer: To a suspension of 9 (2.6 g, 4.6 mmol) in DCM (40.0 mL) was added DCI (0.5 g, 5.6 mmol) and CEP[N(iPr)₂]₂ (1.7 g, 5.6 mmol). The mixture was stirred at r.t. for 1.0 h. LC-MS showed 9 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 28 monomer (3.0 g, 3.9 mmol, 85.2%) as a white solid. ESI-LCMS: m/z 765.3 [M+H]⁺; ¹H-NMR (400 MHz, DMSO-d₆): δ 11.44 (s, 1H), 7.71 (dd, J=8.1, 3.8 Hz, 1H), 5.81 (dd, J=4.4, 2.5 Hz, 1H), 5.74-5.53 (m, 5H), 4.59-4.33 (m, 2H), 4.20-4.14 (m, 1H), 3.88-3.53 (m, 4H), 3.39 (d, J=16.2 Hz, 3H), 2.80 (td, J=5.9, 2.9 Hz, 2H), 1.16 (d, J=1.9 Hz, 30H); ³¹P-NMR (162 MHz, DMSO-d₆): δ 147.68, 149.16, 16.84, 16.55.

### Example 29. Synthesis of Monomer

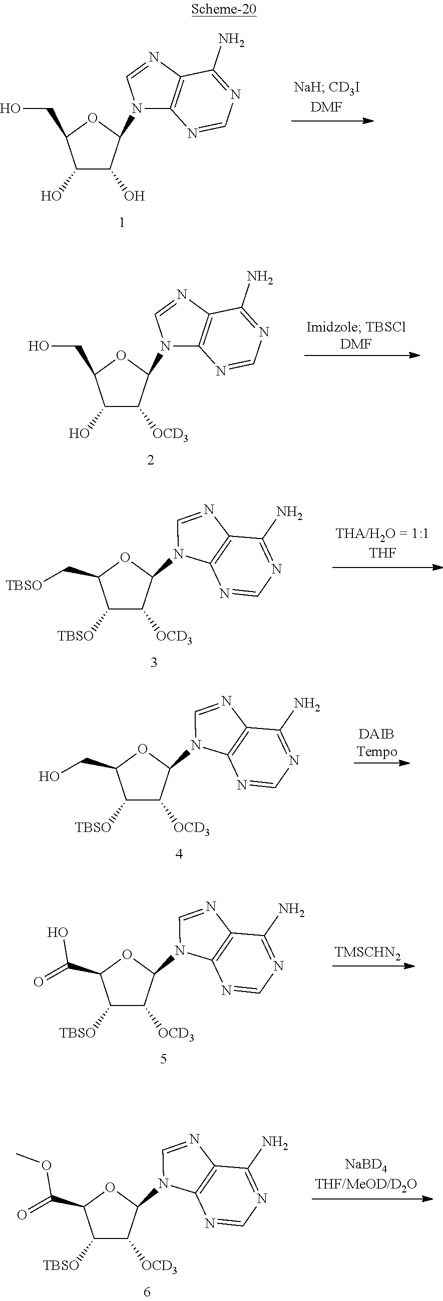

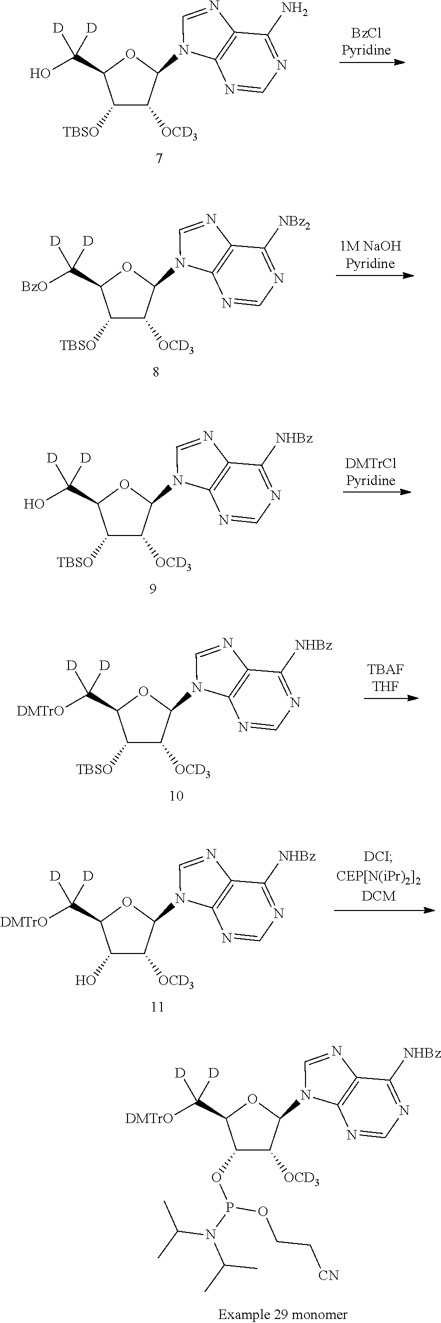

Preparation of (2): To a solution of 1 (26.7 g*2, 0.1 mol) in DMF (400 mL) was added sodium hydride (4.8 g, 0.1 mol) for 30 min, then was added CD₃I (16 g, 0.1 mol) at 0° C. for 2.5 hr (ref. for selective 2′-O-alkylation reaction conditions, J. Org. Chem. 1991, 56, 5846-5859). The mixture was stirring at r.t. for another 1 h. LCMS showed the reaction was consumed. The mixture was filtered and the clear solution was evaporated to dryness and was evaporated with CH₃OH. The crude was purified by silica gel column (SiO₂, DCM/MeOH=50:1˜15:1). This resulted in to give the product 2 (35.5 g, 124.6 mmol, 62% yield) as a solid. ESI-LCMS: m/z 285 [M+H]⁺.

Preparation of (3): To a solution of 2 (35.5 g, 124.6 mmol) in pyridine (360 mL) was added imidazole (29.7 g, 436.1 mmol) and TBSCl (46.9 g, 311.5 mmol). The mixture was stirred at r.t. over night. LCMS showed 2 was consumed completely. The reaction was quenched with water (500 mL). The product was extracted into ethyl acetate (1 L). The organic layer was washed with brine and dried over anhydrous Na₂SO₄. The crude was purified by silica gel column (SiO₂, PE/EA=4:1˜1:1). This resulted in to give the product 3 (20.3 g, 39.6 mmol, 31.8% yield) as a solid. ESI-LCMS: m/z 513 [M+H]⁺; ¹H-NMR (400 MHz, DMSO-d₆): δ 8.32 (m, 1H), 8.13 (m, 1H), 7.31 (m, 2H), 6.02-6.01 (d, J=4.0 Hz, 1H), 4.60-4.58 (m, 1H), 4.49-4.47 (m, 1H), 3.96-3.86 (m, 2H), 3.72-3.68 (m, 1H), 0.91-0.85 (m, 18H), 0.13-0.01 (m, 12H).

Preparation of (4): To a solution of 3 (20.3 g, 39.6 mmol) in THF (80 mL) was added TFA (20 mL) and water (20 mL) at 0° C. The reaction mixture was stirred at 0° C. for 5 h. LC-MS showed 3 was consumed completely. Con. NH₄OH was added to the mixture at 0° C. to quench the reaction until the pH=7.5. The product was extracted into ethyl acetate (200 mL). The organic layer was washed with brine and dried over anhydrous Na₂SO₄. The solution was then concentrated under reduced pressure and the residue was washed by PE/EA=5:1. This resulted in to give 4 (10.5 g, 26.4 mmol, 66.6% yield) as a white solid. ESI-LCMS: m/z 399 [M+H]⁺; ¹H-NMR (400 MHz, DMSO-d₆): δ 8.41 (m, 1H), 8.14 (m, 1H), 7.37 (m, 2H), 5.99-5.97 (d, J=8.0 Hz, 1H), 5.43 (m, 1H), 4.54-4.44 (m, 2H), 3.97-3.94 (m, 1H), 3.70-3.53 (m, 2H), 0.91 (m, 9H), 0.13-0.12 (m, 6H).

Preparation of (5): To a solution of 4 (10.5 g, 26.4 mmol) in ACN/H₂O=1:1 (100 mL) was added DAIB (25.4 g, 79.2 mmol) and TEMPO (1.7 g, 7.9 mmol). The reaction mixture was stirred at 40° C. for 2 h. LCMS showed 4 was consumed. The mixture was diluted with EA and water was added. The product was extracted with EA. The organic layer was washed with brine and dried over anhydrous Na₂SO₄. The solution was then concentrated under reduced pressure and the residue was washed by ACN. This resulted in to give 5 (6.3 g, 15.3 mmol, 57.9% yield) as a white solid. ESI-LCMS: m/z 413 [M+H]⁺; ¹H-NMR (400 MHz, DMSO-d₆): δ=8.48 (m, 1H), 8.16 (m, 1H), 7.41 (m, 2H), 6.12-6.10 (d, J=8.0 Hz, 1H), 4.75-4.73 (m, 1H), 4.42-4.36 (m, 2H), 3.17 (m, 6H), 2.07 (m, 2H), 0.93 (m, 9H), 0.17-0.15 (m, 6H).

Preparation of (6): To a solution of 5 (6.3 g, 15.3 mmol) in toluene (36 mL) and methanol (24 mL) was added (trimethylsilyl)diazomethane (7.0 g, 61.2 mmol) till the yellow color not disappear at r.t. for 2 min. LCMS showed the reaction was consumed. The solvent was removed to give the cured 6 (6.0 g) as a solid which used for the next step. ESI-LCMS: m/z 427 [M+H]⁺; ¹H-NMR (400 MHz, DMSO-d₆): δ 8.45 (m, 1H), 8.15 (m, 1H), 7.35 (m, 2H), 6.12-6.10 (d, J=8.0 Hz, 1H), 4.83-4.81 (m, 1H), 4.50-4.46 (m, 1H), 3.73 (m, 3H), 3.31 (m, 1H), 0.93 (m, 9H), 0.15-0.14 (m, 6H).

Preparation of (7): To the solution of 6 (6 g) in dry THF/MeOD/D₂O=10/2/1 (78 mL) was added NaBD₄ (2.3 g, 54.8 mmol) at r.t. And the reaction mixture was stirred at r.t for 2.5 hr. After completion of reaction, adjusted pH value to 7 with CH₃COOD, after addition of water, the resulting mixture was extracted with EA (100 mL). The combined organic layer was washed with water and brine, dried over Na₂SO₄, and concentrated to give 7 (5.7 g) which was used for the next step. ESI-LCMS: m/z 401 [M+H]⁺.

Preparation of (8): To a solution of 7 (5.7 g) in pyridine (60 mL) was added BzCl (10.0 g, 71.3 mmol) under ice bath. The reaction mixture was stirred at r.t. for 2.5 hrs. LCMS showed 7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=7/3; Detector, UV 254 nm. This resulted in to give the crude 8 (6.2 g, 8.7 mmol, 57% yield, over two steps) as a white solid. ESI-LCMS: m/z 713 [M+H]⁺.

Preparation of (9): To a solution of 8 (6.2 g, 8.7 mmol) in pyridine (70 mL) and was added 1M NaOH (MeOH/H₂O=4/1) (24 mL). LCMS showed 8 was consumed. The mixture was added saturated NH₄Cl till pH=7.5. The mixture was diluted with water and EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=67/33 Detector, UV 254 nm. This resulted in to give the product 10 (4.3 g, 8.5 mmol, 98% yield) as a white solid. ESI-LCMS: m/z 505 [M+H]⁺; ¹H-NMR (400 MHz, DMSO-d₆): δ 11.23 (m, 1H), 8.77 (m, 2H), 8.06-8.04 (m, 2H), 7.66-7.63 (m, 2H), 7.57-7.53 (m, 3H), 6.16-6.14 (d, J=8.0 Hz, 1H), 5.17 (m, 1H), 4.60-4.52 (m, 2H), 3.34 (m, 1H), 0.93 (m, 9H), 0.14 (m, 6H).

Preparation of (10): To a stirred solution of 9 (4.3 g, 8.5 mmol) in pyridine (45 mL) were added DMTrCl (3.3 g, 9.8 mmol) at r.t. And the reaction mixture was stirred at r.t for 2.5 hr. With ice-bath cooling, the reaction was quenched with water and the product was extracted into EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=97/3 Detector, UV 254 nm. This resulted in to give the product 10 (6.5 g, 8.1 mmol, 95% yield) as a white solid. ESI-LCMS: m/z 807 [M+H]⁺; ¹H-NMR (400 MHz, DMSO-d₆): δ 11.23 (m, 1H), 8.70-8.68 (m, 2H), 8.04-8.02 (m, 2H), 7.66-7.62 (m, 1H), 7.56-7.52 (m, 2H), 7.35-7.26 (m, 2H), 7.25-7.17 (m, 7H), 6.85-6.82 (m, 4H), 6.18-6.16 (d, J=8.0 Hz, 1H), 4.73-4.70 (m, 1H), 4.61-4.58 (m, 1H), 3.71 (m, 6H), 3.32 (m, 1H), 0.83 (m, 9H), 0.09-0.03 (m, 6H).

Preparation of (11): To a solution of 10 (3.5 g, 4.3 mmol) in THF (35 mL) was added 1 M TBAF solution (5 mL). The reaction mixture was stirred at r.t. for 1.5 h. LCMS showed 10 was consumed completely. Water (100 mL) was added. The product was extracted with EA (100 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 20 min, the eluted product was collected at CH₃CN/H₂O (0.5% NH₄HCO₃)=62/38; Detector, UV 254 nm. This resulted in to give 11 (2.7 g, 3.9 mmol, 90.7%) as a white solid. ESI-LCMS: m/z 693 [M+H]⁺.

Preparation of Example 29 monomer: To a suspension of 11 (2.7 g, 3.9 mmol) in DCM (30 mL) was added DCI (0.39 g, 3.3 mmol) and CEP[N(iPr)₂]₂ (1.4 g, 4.7 mmol). The mixture was stirred at r.t. for 2 h. LC-MS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=73/27; Detector, UV 254 nm. This resulted in to give Example 29 monomer (3.3 g, 3.7 mmol, 94.9%) as a white solid. ESI-LCMS: m/z 893 [M+H]⁺; ¹H-NMR (400 MHz, DMSO-d₆): δ=11.24 (m, 1H), 8.66-8.64 (m, 2H), 8.06-8.03 (m, 2H), 7.65-7.53 (m, 3H), 7.42-7.38 (m, 2H), 7.37-7.34 (m, 2H), 7.25-7.19 (m, 7H), 6.86-6.80 (m, 4H), 6.20-6.19 (d, J=4.0 Hz, 1H), 4.78 (m, 2H), 4.22-4.21 (m, 1H), 3.92-3.83 (m, 1H), 3.72 (m, 6H), 3.62-3.57 (m, 3H), 2.81-2.78 (m, 1H), 2.64-2.61 (m, 1H), 1.17-1.04 (m, 12H); ³¹P-NMR (162 MHz, DMSO-d₆): δ 149.51, 149.30.

### Example 30. Synthesis of Monomer

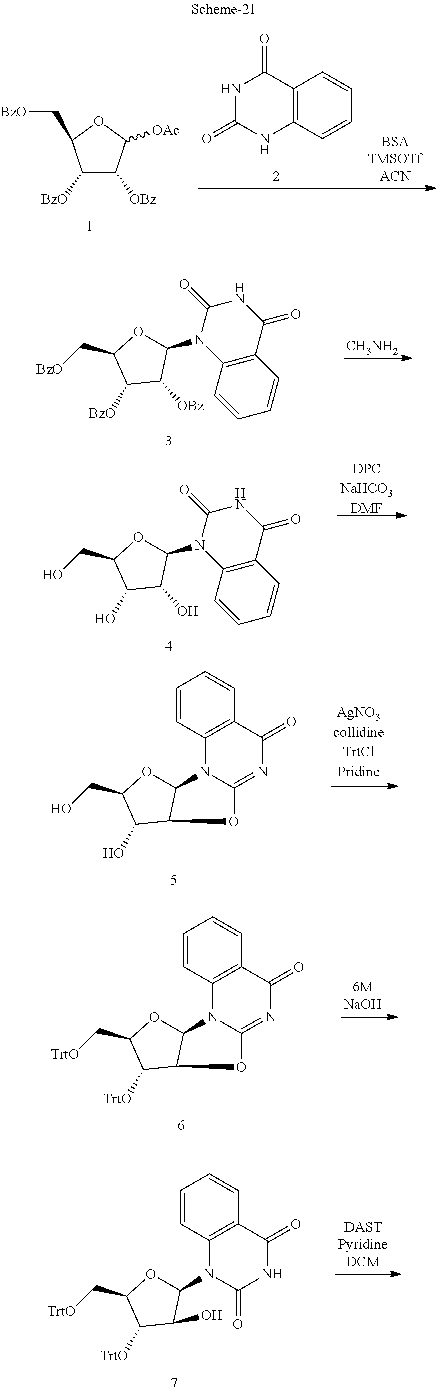

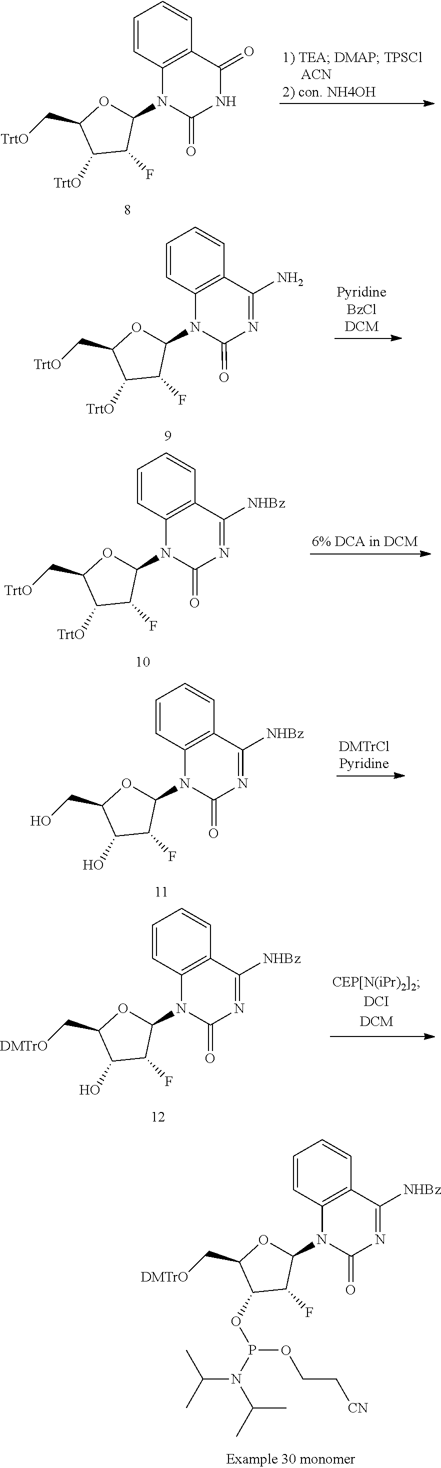

Preparation of (3): To the solution of 1 (70 g, 138.9 mmol) in dry acetonitrile (700 mL) was added 2 (27.0 g, 166.7 mmol), BSA (112.8 g, 555.5 mmol). The mixture was stirred at 50° C. for 1 h. Then the mixture was cooled to −5° C. and TMSOTf (46.2 g, 208.3 mmol) slowly added to the mixture. Then the reaction mixture was stirred at r.t for 48 h. Then the solution was cooled to 0° C. and saturated aq. NaHCO₃ was added and the resulting mixture was extracted with EA. The combined organic layer was washed with water and brine, dried over Na₂SO₄, and concentrated under reduced pressure to give a residue which was purified by silica gel column chromatography (eluent, PE:EA-3:1˜1:1) to give 3 (70 g, 115.3 mmol, 81.6%) as a white solid. ESI-LCMS: m/z 605 [M−H]⁺.

Preparation of (4): To the solution of 3 (70.0 g, 115.3 mmol) in methylammonium solution (1 M, 700 mL), and the reaction mixture was stirred at 40° C. for 15 h. After completion of reaction, the resulting mixture was concentrated. The residue was crystallized from EA. Solid was isolated by filtration, washed with PE and dried overnight at 45° C. in vacuum to give 4 (31.0 g, 105.4 mmol, 91.1%) as a white solid. ESI-LCMS: m/z 295 [M+H]⁺; ¹H-NMR (400 MHz, DMSO): δ 11.63 (s, 1H), 8.07-7.99 (m, 1H), 7.81 (d, J=8.4 Hz, 1H), 7.72-7.63 (m, 1H), 7.34-7.26 (m, 1H), 6.18 (d, J=6.4 Hz, 1H), 5.24 (s, 1H), 5.00 (s, 2H), 4.58-4.47 (m, 1H), 4.19-4.10 (m, 1H), 3.85-3.77 (m, 1H), 3.75-3.66 (m, 1H), 3.66-3.57 (m, 1H).

Preparation of (5): To the solution of 4 (20.0 g, 68.0 mmol) in dry DMF (200 mL) was added DPC (18.9 g, 88.0 mmol) and NaHCO₃ (343 mg, 4 mmol) at r.t, and the reaction mixture was stirred at 150° C. for 35 min. After completion of reaction, the resulting mixture was poured into tert-Butyl methyl ether (4 L). Solid was isolated by filtration, washed with PE and dried in vacuum to give crude 5 (21.0 g) as a brown solid which was used directly for next step (ref for 5, Journal of Organic Chemistry, 1989, vol. 33, p. 1219-1225). ESI-LCMS: m/z 275 [M−H]⁻.

Preparation of (6): To the solution of 5 (crude, 21.0 g) in Pyridine (200 mL) was added AgNO₃ (31.0 g, 180.0 mmol) and collidine (88.0 g, 720 mmol) and TrtCl (41.5 g, 181 mmol) at r.t, and the reaction mixture was stirred at r.t for 15 h. After addition of water, the resulting mixture was extracted with EA. The combined organic layer was washed with water and brine, dried over Na₂SO₄, and concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (10.0 g, 13.1 mmol, 20% yield over 3 steps) as a white solid. ESI-LCMS: m/z 761 [M+H]⁺.

Preparation of (7): To the solution of 6 (10.0 g, 13.1 mmol) in THF (100 mL) was added 6 N NaOH (30 mL) at r.t, and the reaction mixture was stirred at r.t for 1 hr. After addition of NH₄Cl, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in to give 7 (9.3 g, 11.9 mmol, 90%) as a white solid. ESI-LCMS: m/z 777 [M−H]⁻; ¹H-NMR (400 MHz, DMSO-d₆): δ 11.57 (s, 1H), 8.02 (d, J=8.7 Hz, 1H), 7.88-7.81 (m, 1H), 7.39-7.18 (m, 30H), 7.09-6.99 (m, 30H), 6.92-6.84 (m, 30H), 6.44 (d, J=4.0 Hz, 1H), 4.87 (d, J=4.0 Hz, 1H), 4.37-4.29 (m, 1H), 4.00-3.96 (m, 1H), 3.76-3.70 (m, 1H), 3.22-3.13 (m, 1H), 3.13-3.04 (m, 1H).

Preparation of (8): To the solution of 7 (8.3 g, 10.7 mmol) in dry DCM (80 mL) was added Pyridine (5.0 g, 64.2 mmol) and DAST (6.9 g, 42.8 mmol) at 0° C., and the reaction mixture was stirred at r.t for 15 hr. After addition of NH₄Cl, the resulting mixture was extracted with DCM. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 8 (6.8 g, 8.7 mmol, 81.2%) as a white solid. ESI-LCMS: m/z 779 [M−H]⁺; ¹⁹F-NMR (376 MHz, DMSO-d₆): δ −183.05.

Preparation of (9): To the solution of 8 (5.8 g, 7.5 mmol) in dry ACN (60 mL) was added TEA (1.5 g, 15.1 mmol), DMAP (1.84 g, 15.1 mmol) and TPSCl (4.1 g, 13.6 mmol) at r.t, and the reaction mixture was stirred at room temperature for 3 h under N₂ atmosphere. After completion of reaction, the mixture was added NH₃·H₂O (12 mL). After addition of water, the resulting mixture was extracted with EA. The combined organic layer was washed with water and brine, dried over Na₂SO₄, and concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 9 (5.5 g, 7 mmol, 90.2%) as a white solid. ESI-LCMS: m/z 780 [M+H]⁺.

Preparation of (10): To a solution of 9 (5.5 g, 7 mmol) in DCM (50 mL) with an inert atmosphere of nitrogen was added pyridine (5.6 g, 70.0 mmol) and BzCl (1.2 g, 8.5 mmol) in order at 0° C. The reaction solution was stirred for 30 minutes at room temperature. The solution was diluted with DCM (100 mL) and the combined organic layer was washed with water and brine, dried over Na₂SO₄, and concentrated under reduced pressure to give a residue which was purified by silica gel column chromatography (eluent, PE:EA=5:1˜2:1) to give 10 (5.4 g, 6.1 mmol, 90.6%) as a white solid. ESI-LCMS: m/z 884 [M+H]⁺; ¹⁹F-NMR (376 MHz, DMSO-d₆): δ −183.64.

Preparation of (11): To the solution of 10 (5.4 g, 6.1 mmol) in the solution of DCA (6%) in DCM (60 mL) was added TES (15 mL) at r.t, and the reaction mixture was stirred at room temperature for 5-10 min. After completion of reaction, the resulting mixture was added NaHCO₃, the resulting mixture was extracted with DCM. The combined organic layer was washed with water and brine, dried over Na₂SO₄, and concentrated under reduced pressure and the residue was crystallized from EA. Solid was isolated by filtration, washed with PE and dried overnight at 45° in vacuum to give 11 (2.0 g, 5.0 mmol, 83.2%) as a white solid. ESI-LCMS: m/z 400 [M+H]⁺.

Preparation of (12): To a solution of 11 (2.0 g, 5.0 mmol) in dry Pyridine (20 mL) was added DMTrCl (2.0 g, 6.0 mmol). The reaction mixture was stirred at r.t. for 2.5 h. LCMS showed 11 was consumed and water (200 mL) was added. The product was extracted with EA (200 mL) and the organic layer was washed with brine and dried over Na₂SO₄ and concentrated to give the crude. The crude was purified by c.c. (PE:EA=4:1˜1:1) to give crude 12. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 12 (2.1 g, 3 mmol, 60%) as a white solid. ESI-LCMS: m/z 702 [M+H]⁺; ¹H-NMR (400 MHz, DMSO-d₆): δ 12.63 (s, 1H), 8.54 (d, J=7.8 Hz, 1H), 8.25 (d, J=7.2 Hz, 2H), 7.82 (d, J=3.6 Hz, 2H), 7.67-7.58 (m, 1H), 7.57-7.49 (m, 2H), 7.49-7.39 (m, 1H), 7.39-7.31 (m, 2H), 7.27-7.09 (m, 7H), 6.82-6.69 (m, 4H), 6.23 (d, J=26.1 Hz, 1H), 5.59-5.49 (m, 1H), 4.83-4.61 (m, 1H), 4.15-4.01 (m, 1H), 3.74-3.59 (m, 6H), 3.33-3.28 (m, 1H), 3.16-3.05 (m, 1H). ¹⁹F-NMR (376 MHz, DMSO-d₆): δ −191.66.

Preparation of Example 30 monomer: To a suspension of 12 (2.1 g, 3.0 mmol) in DCM (20 mL) was added DCI (310 mg, 2.6 mmol) and CEP[N(iPr)₂]₂ (1.1 g, 3.7 mmol). The mixture was stirred at r.t. for 1 h. LC-MS showed 12 was consumed completely. The solution was washed with water twice and washed with brine and dried over Na₂SO₄. Then concentrated to give the crude. The crude was by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 30 monomer (2.1 g, 2.3 mmol, 80.0%) as a white solid. ESI-LCMS: m/z 902 [M+H]⁺. ¹H-NMR (400 MHz, DMSO-d₆): δ 12.64 (s, 1H), 8.54 (d, J=7.6 Hz, 1H), 8.24 (d, J=7.7 Hz, 2H), 7.93-7.88 (m, 2H), 7.67-7.58 (m, 1H), 7.56-7.42 (m, 3H), 7.41-7.29 (m, 2H), 7.27-7.08 (m, 7H), 6.82-6.64 (m, 4H), 6.37-6.18 (m, 1H), 6.03-5.72 (m, 1H), 5.26-4.83 (m, 1H), 4.28-4.12 (m, 1H), 3.88-3.72 (m, 1H), 3.71-3.37 (m, 9H), 3.15-3.00 (m, 1H), 2.83-2.75 (m, 1H), 2.66-2.57 (m, 1H), 1.21-0.88 (m, 12H). ¹⁹F-NMR (376 MHz, DMSO-d₆): δ −189.71. ³¹P-NMR (162 MHz, DMSO-d₆): δ 149.48, 149.50, 148.95, 148.88.

### Example 31. Synthesis of Monomer

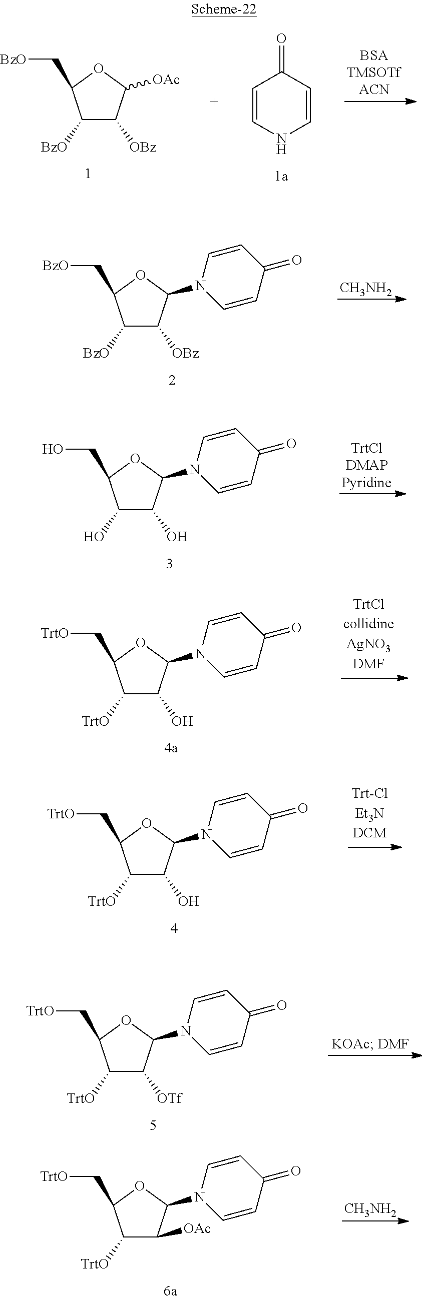

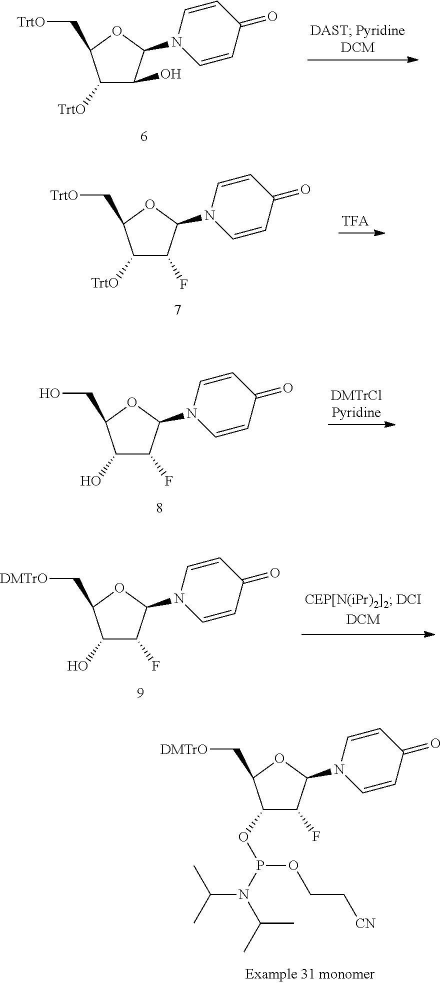

Preparation of (2): To a solution of 1 (40.0 g, 79.3 mmol), 1a (7.6 g, 80.1 mmol) in ACN (100 mL). Then added BSA (35.2 g, 174.4 mmol) under N₂ atmosphere. The mixture was stirred at 50° C. for 1 h until the solution was clear. Then cool down to 0° C. and dropped TMSOTf (18.5 g, 83.2 mmol). The mixture was stirred at 75° C. for 1 h, TLC showed 1 was consumed completely. Then the solution was diluted with EA, washed with H₂O twice. The solvent was concentrated under reduced pressure and the residue was used for next step. ESI-LCMS: m/z 540 [M+H]⁺.

Preparation of (3): To a solution of 2 (37.1 g, 68.7 mmol) in 30% CH₂NH₂/MeOH solution (200 mL). The mixture was stirred at 25° C. for 2 h. TLC showed 2 was consumed completely. The solvent was concentrated under reduced pressure and the residue was washed with EA twice to give 3 (12.5 g, 55.2 mmol) (ref. for intermediate 3 Bioorganic & Medicinal Chemistry Letters, 1996, Vol. 6, No. 4, pp. 373-378) which was used directly for the next step. ESI-LCMS: m/z 228 [M+H]⁺.

Preparation of (4): To a solution of 3 (12.5 g, 55.2 mmol) in pyridine (125 mL) and added DMAP (1.3 g, 11.0 mmol), TrtCl (30.7 g, 110.5 mmol). The mixture was stirred at r.t. for 24 h. TLC showed 3 was consumed completely. H₂O was added to the mixture. Then filtered and the solution diluted with EA. The organic layer was washed with NaHCO₃ and brine. The solvent was concentrated under reduced pressure and then added ACN, filtered to give 4a (17.0 g, 35.4 mmol, 64% yield) as a white solid.

To a solution of 4a (17.0 g, 35.4 mmol) in DMF (200 mL), collidine (5.2 g, 43.5 mmol), TrCl (13.1 g, 47.1 mmol) were added after 2 h and then again after 3 h TrCl (13.1 g, 47.1 mmol), AgNO3 (8.0 g, 47.1 mmol). The mixture was stirred at 25° C. for 24 h. TLC showed 4a was consumed completely. Then filtered and the solution diluted with EA. The organic layer was washed with NaHCO₃ and brine. The solvent was concentrated under reduced pressure and then added ACN, filtered to get 4 (14.2 g, 19.5 mmol, 54% yield) as a white solid. ESI-LCMS: m/z 712 [M+H]⁺; ¹H-NMR (400 MHz, DMSO-d₆): δ 7.83 (d, J=8 Hz, 2H), 7.42-7.20 (m, 30H), 6.18 (d, J=7 Hz, 1H), 6.09 (d, J=8 Hz, 2H), 5.60 (d, J=7 Hz, 1H), 4.22 (m, 1H), 3.90 (d, J=5 Hz, 1H), 2.85 (d, J=10 Hz, 1H), 2.76 (s, 1H), 2.55-2.50 (dd, 1H).

Preparation of (5): To a solution of 4 (14.2 g, 19.9 mmol) in DCM (150 mL), DMAP (2.4 g, 19.9 mmol), TEA (4.0 g, 39.9 mmol, 5.6 mL) were added. Then cool down to 0° C., TfCl (6.7 g, 39.9 mmol) dissolved in DCM (150 mL) were dropped. The mixture was stirred at 25° C. for 1 h. TLC showed 4 was consumed completely. Then filtered and the solution diluted with EA. The organic layer was washed with NaHCO₃ and brine. The solvent was concentrated under reduced pressure to get 5 (16.8 g, 19.9 mmol) as a brown solid. ESI-LCMS: m/z 844 [M+H]⁺.

Preparation of (6): To a solution of 5 (16.8 g, 19.9 mmol) in DMF (200 mL), KOAc (9.7 g, 99.6 mmol) were added, The mixture was stirred at 25° C. for 14 h and 50° C. for 3 h, TLC showed 5 was consumed completely. Then filtered and the solution diluted with EA. The organic layer was washed with H₂O and brine. The solvent was concentrated under reduced pressure to get 6a (15.0 g, 18.9 mmol, 90% yield) as a brown solid. To a solution of 6a (15.0 g, 19.9 mmol) in 30% CH₃NH₂/MeOH solution (100 mL) were added. The mixture was stirred at 25° C. for 2 h, TLC showed 6a was consumed completely. Then the solvent was concentrated under reduced pressure and the residue was purified by cc (0-5% MeOH in DCM) to give 6 (11.6 g, 16.3 mmol, 82% yield) as a yellow solid. ESI-LCMS: m/z 712 [M+H]⁺; ¹H-NMR (400 MHz, DMSO-d₆): δ 7.59 (d, J=8 Hz, 2H), 7.37-7.22 (m, 30H), 6.01 (d, J=8 Hz, 2H), 5.84 (d, J=3 Hz, 1H), 5.42 (d, J=4 Hz, 1H), 3.78-3.70 (m, 3H), 3.10 (t, J=9 Hz, 1H), 2.53 (d, J=4 Hz, 6H), 1.77 (s, 6H).

Preparation of (7): To a solution of 6 (11.6 g, 16.32 mmol) in DCM (200 mL), DAST (7.9 g, 48.9 mmol) were added at 0° C., The mixture was stirred at 25° C. for 16 h, TLC showed 6 was consumed completely. Then the solution was diluted with EA, washed with NaHCO₃ twice, The solvent was concentrated under reduced pressure the residue purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in to give 7 (11.6 g, 13.8 mmol, 84% yield) as a white solid. ESI-LCMS: m/z 714 [M+H]⁺.

Preparation of (8): To a solution of 7 (11.6 g, 16.2 mmol) in DCM (100 mL) was added TFA (10 mL). The mixture was stirred at 20° C. for 1 h. TLC showed 7 was consumed completely. Then the solution was concentrated under reduced pressure the residue was purified by silica gel column (0˜20% MeOH in DCM) and Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=0/1; Detector, UV 254 nm. This resulted in to give 9 (1.7 g, 7.2 mmol, 45% yield) as a white solid. ESI-LCMS: m/z 229.9 [M+H]⁺; ¹H-NMR (400 MHz, DMSO-d₆): δ 7.91 (d, J=8 Hz, 2H), 6.14 (d, J=8 Hz, 2H), 5.81-5.76 (m, 2H), 5.28 (t, J=5 Hz, 1H), 5.13-4.97 (t, J=4 Hz, 1H), 4.23 (m, 1H), 3.97 (m, 1H), 3.74-3.58 (m, 2H); ¹⁹F-NMR (376 MHz, DMSO-d₆): δ −206.09.

Preparation of (9): To a solution of 8 (1.4 g, 6.1 mmol) in pyridine (14 mL) was added DMTrCl (2.5 g, 7.3 mmol) at 20° C. The mixture was stirred at 20° C. for 1 h. TLC showed 8 was consumed completely. Water was added to the reaction. The product was extracted with EA, The organic layer was washed with NaHCO₃ and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=1/1; Detector, UV 254 nm. This resulted in to give 9 (2.5 g, 4.6 mmol, 76 yield) as a white solid. ESI-LCMS: m/z 532.2 [M+H]⁺; ¹H-NMR (400 MHz, DMSO-d₆): δ 7.87-7.84 (m, 2H), 7.40-7.22 (m, 9H), 6.91-6.87 (m, 4H), 5.98-5.95 (m, 2H), 5.88-5.77 (m, 2H), 5.16-5.02 (m, 1H), 4.42 (m, 1H), 4.05 (m, 1H), 3.74 (s, 6H), 3.35 (m, 2H); ¹⁹F-NMR (376 MHz, DMSO-d₆): δ −202.32.

Preparation of Example 31 monomer: To a solution of 9 (2.2 g, 4.1 mmol) in DCM (20 mL) was added DCI (415 mg, 3.5 mmol) and CEP (1.5 g, 4.9 mmol) under N₂ pro. The mixture was stirred at 20° C. for 0.5 h. TLC showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 31 monomer (2.6 g, 3.5 mmol, 85% yield) as a white solid. ESI-LCMS: m/z 732.2 [M+H]⁺; ¹H-NMR (400 MHz, DMSO-d₆): δ 7.87-7.84 (m, 2H), 7.40-7.22 (m, 9H), 6.91-6.87 (m, 4H), 5.98-5.95 (m, 2H), 5.90-5.88 (m, 1H), 5.30-5.17 (m, 1H), 4.62 (m, 1H), 4.19 (m, 1H), 3.78-3.73 (m, 7H), 3.62-3.35 (m, 5H), 2.78 (t, J=5 Hz, 1H), 2.63 (t, J=6 Hz, 1H), 1.14-0.96 (m, 12H); ¹⁹F-NMR (376 MHz, DMSO-d₆): δ −200.77, 200.80, 201.62, 201.64. ³¹P-NMR (162 MHz, DMSO-d₆): δ 150.31, 150.24, 149.66, 149.60.

### Example 32. Synthesis of End Cap Monomer

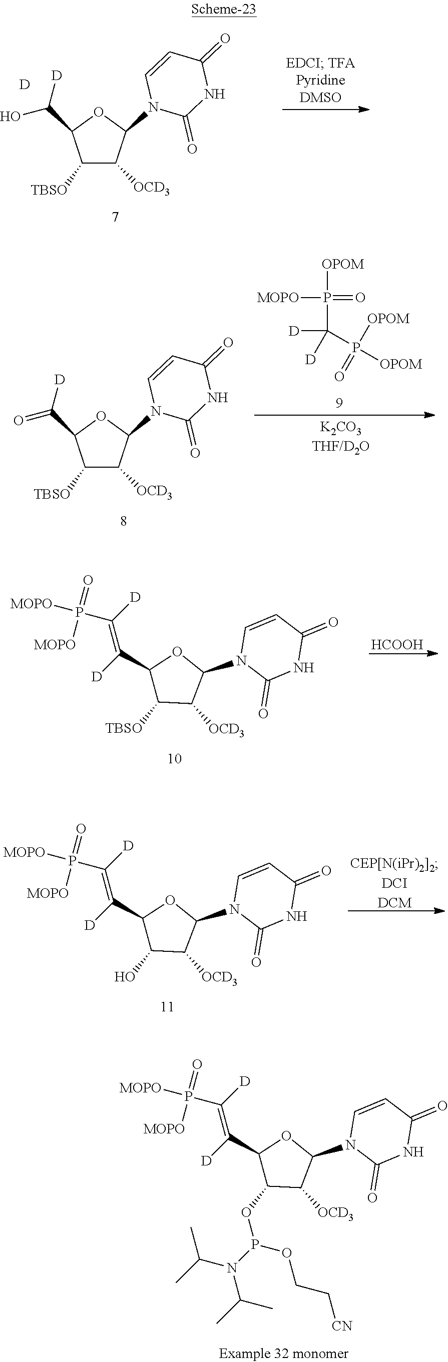

Preparation of (8): To a stirred solution of 7 (13.4 g, 35.5 mmol, Scheme 5) in DMSO (135 mL) were added EDCI (6.3 g, 32.9 mmol) and pyridine (0.9 g, 10.9 mmol), TFA (0.6 g, 5.5 mmol) at r.t. And the reaction mixture was stirred at r.t for 2 h. LCMS showed 7 consumed completely. The reaction was quenched with water and the product was extracted with EA (1800 mL). The organic phase was washed by brine, dried over Na₂SO₄, The organic phase was evaporated to dryness under reduced pressure to give a residue 8 (13.2 g, 35.3 mmol, 99.3% yield). Which was used directly to next step. ESI-LCMS: m/z=375 [M+H₂O]⁺

Preparation of (10): A solution of 8 (13.2 g, 35.3 mmol), 9 (26.8 g, 42.3 mmol, Scheme 18) and K₂CO₃ (19.5 g, 141.0 mmol) in dry THF (160 mL) and D₂O (53 mL) was stirred at r.t. 17 h. LCMS showed most of 8 was consumed. The product was extracted with EA (2500 mL) and the organic layer was washed with brine and dried over Na₂SO₄. Then the organic layer was concentrated to give a residue which was purified by c.c. (PE:EA=10:1 ˜1:2) to give product 10 (8.1 g, 11.8 mmol, 33.4% yield) as a white solid. ESI-LCMS m/z=682 [M+H]⁺; ¹H-NMR (400 MHz, DMSO-d₆): δ 11.42 (s, 1H), 7.69-7.71 (d, J=8.1 Hz, 1H), 5.78-5.79 (d, J=3.7 Hz, 1H), 5.65-5.67 (m, 1H), 5.59-5.63 (m, 4H), 4.29-4.35 (m, 2H), 3.97-3.99 (m, 1H), 1.15 (s, 18H), 0.87 (s, 9H), 0.07-0.08 (d, J=5.1 Hz, 6H). ³¹P-NMR (162 MHz, DMS O-d₆) δ 16.62.

Preparation of (11): To a round-bottom flask was added 10 (7.7 g, 11.1 mmol) in a mixture of HCOOH (80 mL) and H₂O (80 mL). The reaction mixture was stirred at 40° C. for 3 h. LCMS showed the 10 was consumed completely. The reaction mixture was adjusted the pH=7.0 with con·NH₃·H₂O (100 mL). Then the mixture was extracted with DCM (100 mL*3). The combined DCM layer was dried over Na₂SO₄. Filtered and filtrate was concentrated to give crude which was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/1 within 20 min, the eluted product was collected at CH₃CN/H₂O (0.5% NH₄HCO₃)=1/0; Detector, UV 254 nm. To give product 11 (5.5 g, 9.6 mmol, 86.1% yield) as a white solid. ESI-LCMS m/z=568 [M+H]⁺; ¹H-NMR (400 MHz, DMSO-d₆): δ 11.42 (s, 1H, exchanged with D₂O), 7.62-7.64 (d, J=8.1, 1H), 5.81-5.82 (d, J=4.3, 1H), 5.58-5.66 (m, 5H), 5.52-5.53 (d, J=6.6, 1H), 4.34-4.37 (m, 1H), 4.09-4.13 (m, 1H), 3.94-3.96 (t, J=9.7, 1H), 1.15 (s, 18H), 0 (s, 1H). ³¹P NMR (162 MHz, DMSO-d₆) δ 17.16.

Preparation of Example 32 monomer: To a solution of 11 (5.3 g, 9.3 mmol) in DCM (40 mL) was added the DCI (1.1 g, 7.9 mmol), then CEP[N(ipr)₂]₂ (3.4 g, 11.2 mmol) was added. The mixture was stirred at r.t. for 1 h. LCMS showed 11 consumed completely. The reaction mixture was washed with H₂O (50 mL*2) and brine (50 mL*1). Dried over Na₂SO₄ and concentrated to give crude which was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. The product was concentrated to give Example 32 monomer (6.2 g, 8.0 mmol, 85.6% yield) as a white solid. ESI-LCMS m/z=768 [M+H]⁺; ¹H-NMR (400 MHz, DMSO-d₆): δ 11.43 (s, 1H), 7.68-7.71 (m, 1H), 5.79-5.81 (m, 1H), 5.58-5.67 (m, 5H), 4.34-4.56 (m, 2H), 4.14-4.17 (m, 1H), 3.54-3.85 (m, 4H), 2.78-2.81 (m, 2H), 1.13-1.17 (m, 30H). ³¹P-NMR (162 MHz, DMSO-d₆): δ 149.66, 149.16, 16.84, 16.56.

### Example 33. Synthesis of Monomer

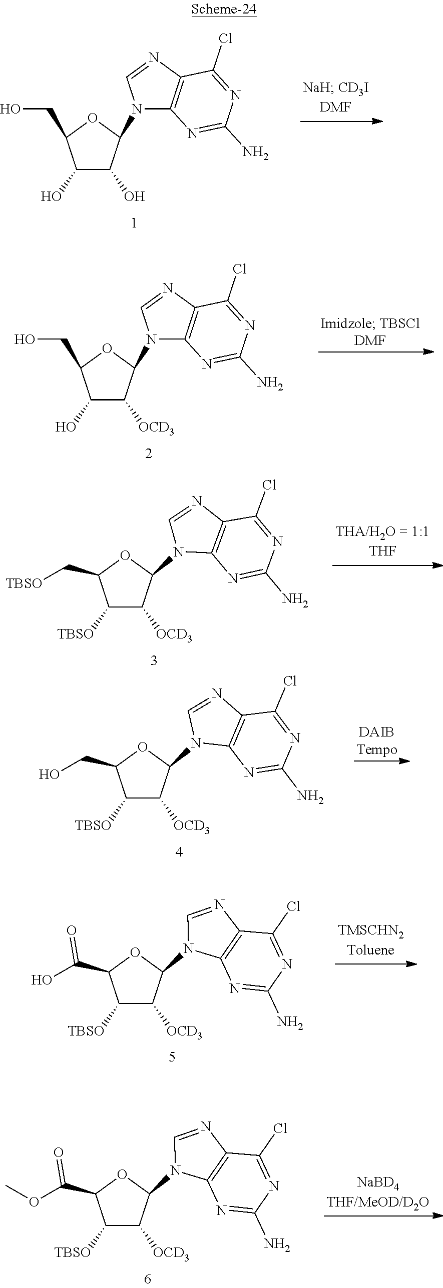

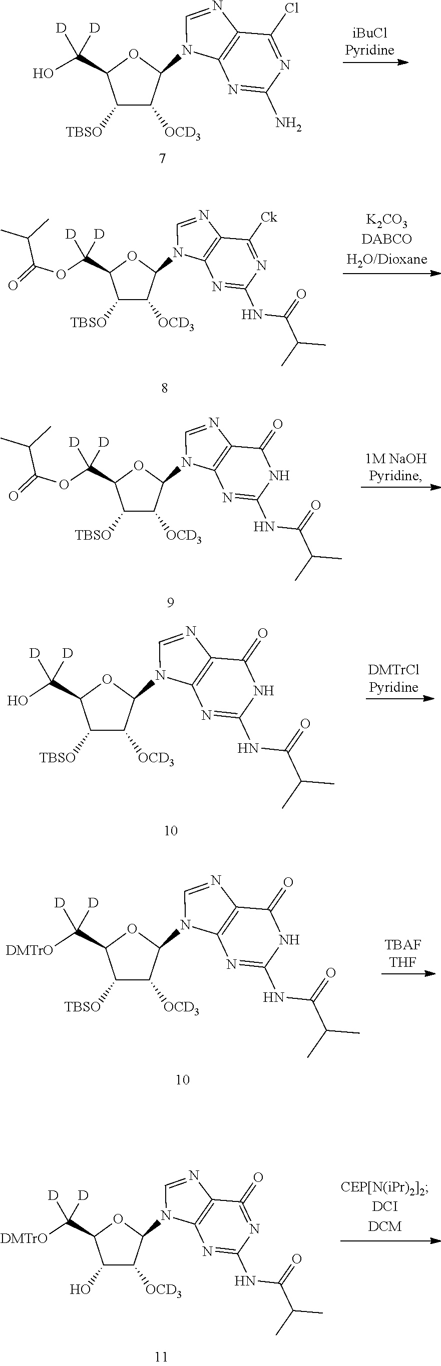

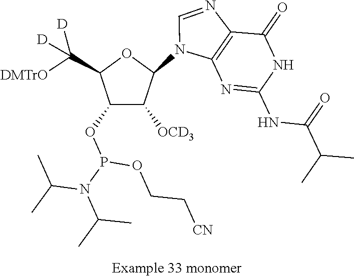

Preparation of (2): To a solution of 1 (20.0 g, 66.4 mmol) in dry DMF (400 mL) was added sodium hydride (1.9 g, 79.7 mmol) for 30 min, then was added CD₃I (9.1 g, 79.7 mmol) in dry DCM (40 mL) at −20° C. for 5.5 hr. LCMS showed the reaction was consumed. The mixture was filtered and the clear solution was evaporated to dryness and was evaporated with CH₃OH. The crude was purified by silica gel column (SiO₂, DCM/MeOH=50:1˜10:1). This resulted in to give the product 2 (7.5 g, 23.5 mmol, 35.5% yield) as a solid. ESI-LCMS: m/z 319 [M+H]⁺; ¹H-NMR (400 MHz, DMSO-d₃): δ=8.38 (m, 1H), 6.97 (m, 2H), 5.93-5.81 (m, 1H), 5.27-5.26 (d, J=4 Hz, 1H), 5.13-5.11 (m, 1H), 4.39-4.31 (m, 1H), 4.31-4.25 (m, 1H), 3.96-3.94 (m, 1H), 3.66-3.63 (m, 1H), 3.63-3.56 (m, 1H).

Preparation of (3): To a solution of 2 (7.5 g, 23.5 mmol) in dry DMF (75 mL) was added Imidazole (5.6 g, 82.3 mmol) and TBSCl (8.9 g, 58.8 mmol). The mixture was stirred at r.t. over night. LCMS showed 2 was consumed completely. The reaction was quenched with water (300 mL). The product was extracted into ethyl acetate (100 mL). The organic layer was washed with brine and dried over anhydrous Na₂SO₄. The solvent was removed to give the cured 3 (9.8 g) as a solid which used for the next step. ESI-LCMS: m/z 547 [M+H]⁺.

Preparation of (4): To a solution of 3 (9.8 g) in THF (40 mL) was added TFA (10 mL) and water (10 mL) at 0° C. The reaction mixture was stirred at 0° C. for 5 h. LC-MS showed 3 was consumed completely. Con. NH₄OH was added to the mixture at 0° C. to quench the reaction until the pH=7.5. The product was extracted into ethyl acetate (200 mL). The organic layer was washed with brine and dried over anhydrous Na₂SO₄. The solvent was removed to give the cured 4 (8.4 g) as a solid which used for the next step. ESI-LCMS: m/z 433 [M+H]⁺.

Preparation of (5): To a solution of 4 (8.4 g) in DCM/H₂O=2:1 (84 mL) was added DAIB (18.8 g, 58.4 mmol) and TEMPO (0.87 g, 5.8 mmol). The reaction mixture was stirred at 40° C. for 2 h. LCMS showed 4 was consumed. The mixture was diluted with DCM and water was added. The product was extracted with DCM. The organic layer was washed with brine and dried over anhydrous Na₂SO₄. The solution was then concentrated under reduced pressure. This resulted in to give 5 (14.4 g) as a white solid. ESI-LCMS: m/z 447 [M+H]⁺.

Preparation of (6): To a solution of 5 (14.4 g) in toluene (90 mL) and methanol (60 mL) was added 2M TMSCHN₂ (8.9 g, 78.1 mmol) till the yellow color not disappear at r.t. for 10 min. LCMS showed 5 was consumed. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=65/35 Detector, UV 254 nm. This resulted in to give the product 6 (3.5 g, 7.6 mmol, 32.3% yield over three steps, 70% purity) as a white solid. ESI-LCMS: m/z 461 [M+H]⁺.

Preparation of (7): To the solution of 6 (3.5 g, 7.6 mmol) in dry THF/MeOD/D₂O=10/2/1 (45 mL) was added NaBD₄ (0.96 g, 22.8 mmol). And the reaction mixture was stirred at r.t for 2.5 hr. After completion of reaction, the resulting mixture was added CH₃COOD to pH=7, after addition of water, the resulting mixture was extracted with EA (100 mL). The combined organic layer was washed with water and brine, dried over Na₂SO₄, and concentrated to give 7 (3.3 g) which was used for the next step. ESI-LCMS: m/z 435 [M+H]⁺.

Preparation of (8): To a solution of 7 (3.3 g) in dry DCM (30 mL) was added pyridine (5.9 g, 74.5 mmol) and iBuCl (2.4 g, 22.4 mmol) in DCM (6 mL) under ice bath. The reaction mixture was stirred at 0° C. for 2.5 hr. LCMS showed 7 was consumed. The mixture was diluted with EA and water was added. The product was extracted with EA. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=87/13; Detector, UV 254 nm. This resulted in to give the crude 8 (1.6 g, 2.8 mmol, 36.8% yield over two steps) as a white solid. ESI-LCMS: m/z 575 [M+H]⁺.

Preparation of (9): To a solution of 8 (1.6 g, 2.8 mmol) in H₂O/dioxane=1:1 (30 ml) was added K₂CO₃ (772.8 mg, 5.6 mmol) and DABCO (739.2 mg, 2.9 mmol). The reaction mixture was stirred at 50° C. for 3 hr. LCMS showed 8 was consumed. The mixture was diluted with EA and water was added. The product was extracted with EA. The combined organic layer was washed with water and brine, dried over Na₂SO₄, and concentrated to give 9 (1.8 g) which was used for the next step. ESI-LCMS: m/z 557 [M+H]⁺.

Preparation of (10): To a solution of 9 (1.8 g) in pyridine (20 mL) and was added 2M NaOH (MeOH/H₂O=4/1) (5 mL) at 0° C. for 1 h. LCMS showed 9 was consumed. The mixture was added saturated NH₄Cl till pH=7.5. The mixture was diluted with water and EA. The organic layer was washed with brine and dried over Na₂SO₄ and concentrated to give the crude. This resulted in to give the product 10 (1.5 g) as a white solid which was used for the next step. ESI-LCMS: m/z 487 [M+H]⁺.

Preparation of (11): To a stirred solution of 10 (1.5 g) in pyridine (20 mL) were added DMTrCl (1.1 g, 3 mmol) at r.t. And the reaction mixture was stirred at r.t for 2.5 hr. With ice-bath cooling, the reaction was quenched with water and the product was extracted into EA. The organic layer was washed with brine and dried over Na₂SO₄ and concentrated to give the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=7/3 Detector, UV 254 nm. This resulted in to give the product 11 (1.9 g, 2.4 mmol, 85.7% yield over two steps) as a white solid. ESI-LCMS: m/z 789.3 [M+H]⁺; ¹H-NMR (400 MHz, DMSO-d₆): δ 12.10 (m, 1H), 11.63 (m, 1H), 8.20 (m, 1H), 7.35-7.33 (m, 2H), 7.29-7.19 (m, 7H), 6.86-6.83 (m, 4H), 5.89-5.88 (d, J=4 Hz, 1H), 4.40-4.28 (m, 2H), 3.72 (m, 6H), 2.81-2.76 (m, 1H), 1.13-1.11 (m, 6H), 0.80 (m, 9H), 0.05-0.01 (m, 7H).

Preparation of (12): To a solution of 11 (1.9 g, 2.4 mmol) in THF (20 mL) was added 1 M TBAF solution (3 mL). The reaction mixture was stirred at r.t. for 1.5 h. LCMS showed 11 was consumed completely. Water (100 mL) was added. The product was extracted with EA (50 mL) and the organic layer was washed with brine and dried over Na₂SO₄. Then the organic layer was concentrated to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=1/0 within 20 min, the eluted product was collected at CH₃CN/H₂O (0.5% NH₄HCO₃)=58/42; Detector, UV 254 nm. This resulted in to give 12 (1.5 g, 2.2 mmol, 91.6% yield) as a white solid. ESI-LCMS: m/z 675.3 [M+H]⁺; ¹H-NMR (400 MHz, DMSO-d₆): δ 12.09 (m, 1H), 11.60 (m, 1H), 8.14 (m, 1H), 7.35-7.27 (m, 2H), 7.25-7.20 (m, 7H), 6.85-6.80 (m, 4H), 5.96-5.94 (d, J=8 Hz, 1H), 5.26-5.24 (m, 1H), 4.35-4.28 (m, 2H), 3.72 (m, 6H), 3.32 (m, 1H), 2.79-2.72 (m, 1H), 1.13-1.11 (m, 6H).

Preparation of Example 33 monomer: To a suspension of 11 (1.5 g, 2.2 mmol) in DCM (15 mL) was added DCI (220.8 mg, 1.9 mmol) and CEP[N(iPr)₂]₂ (795.7 mg, 2.6 mmol) under N₂ pro. The mixture was stirred at r.t. for 2 h. LCMS showed 11 was consumed completely. The solution was washed with water twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=4/1; Detector, UV 254 nm. This resulted in to give Example 33 monomer (1.6 g, 1.8 mmol, 83% yield) as a white solid. ESI-LCMS: m/z 875 [M+H]⁺; ¹H-NMR (400 MHz, DMSO-d₆): δ 12.12 (m, 1H), 11.60 (m, 1H), 8.15 (m, 1H), 7.37-7.29 (m, 2H), 7.27-7.20 (m, 7H), 6.86-6.81 (m, 4H), 5.94-5.88 (m, 1H), 4.54-4.51 (m, 2H), 4.21-4.20 (m, 1H), 3.73-3.54 (m, 10H), 2.80-2.75 (m, 1H), 2.61-2.58 (m, 1H), 1.19-1.11 (m, 19H). ³¹P-NMR (162 MHz, DMSO-d₆): δ=149.77, 149.71.

### Example 34. Synthesis of Monomer

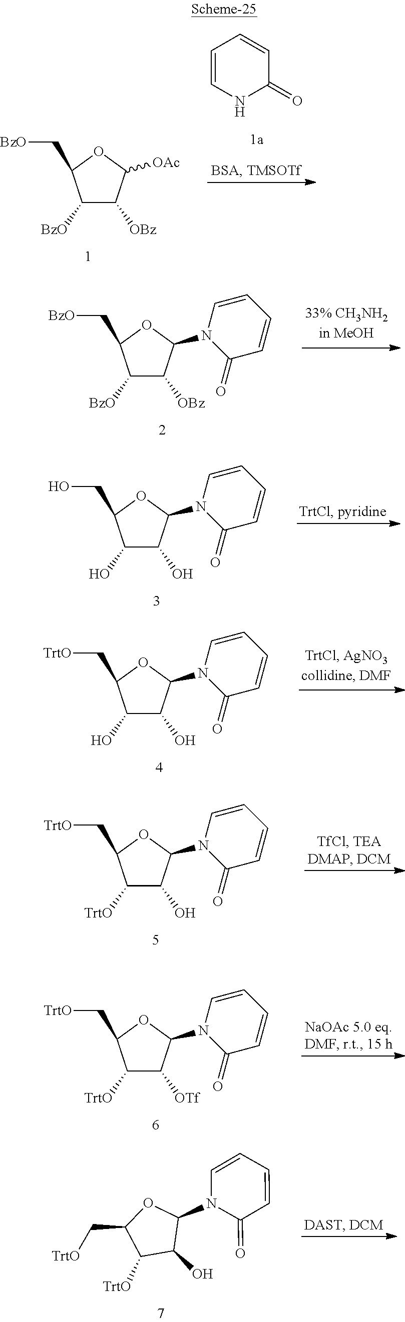

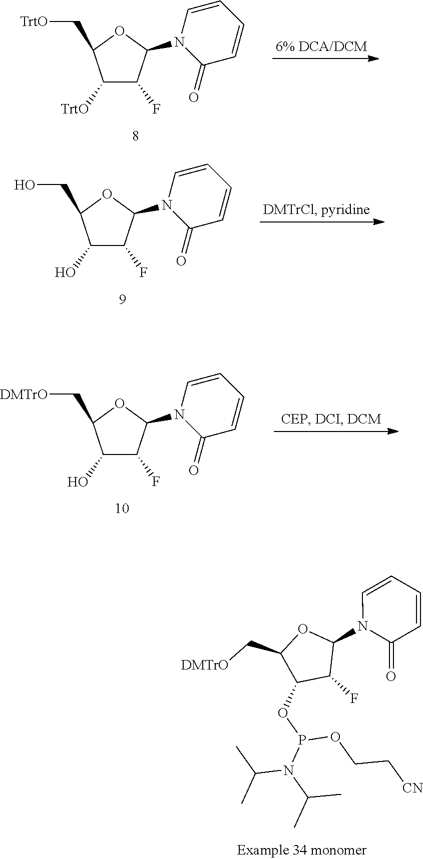

Preparation of (2): To a solution of 1 (50.0 g, 99.2 mmol) and 1a (11.3 g, 119.0 mmol) in ACN (500.0 mL). Then added BSA (53.2 g, 218.0 mmol) under N₂ Pro. The mixture was stirred at 50° C. for 1 h until the solution was clear. Then cool down to 0° C. and dropped TMSOTf (26.4 g, 119.0 mmol). The mixture was stirred at 75° C. for 1 h, TLC showed 1 was consumed completely. The reaction was quenched by sodium bicarbonate solution at 0° C., then the solution was diluted with EA, washed with H₂O twice. The solvent was concentrated under reduced pressure and the crude 2 (60.1 g) was used for next step. ESI-LCMS: m/z 540.2 [M+H]⁺.

Preparation of (3): To a solution of 2 (60.1 g) in CH₃NH₂/ethanol (500.0 mL). The mixture was stirred at 25° C. for 2 h. TLC showed 2 was consumed completely. The solvent was concentrated under reduced pressure and the residue was purified by c.c. (MeOH:DCM=50:1 ˜10:1) to give 3 (22.0 g, 96.9 mmol, 97.3% yield over two steps). ESI-LCMS: m/z 228.0 [M+H]⁺; ¹H-NMR (400 MHz, DMSO-d₆): δ 8.01-7.98 (m, 1H), 7.43-7.38 (m, 1H), 6.37-6.35 (m, 1H), 6.27-6.23 (m, 1H), 6.03 (d, J=3.5 Hz, 1H), 5.39 (d, J=4.2 Hz, 1H), 5.11 (t, J=5.1 Hz, 1H), 5.03 (d, J=5.1 Hz, 1H), 3.98-3.95 (m, 2H), 3.91-3.88 (m, 1H), 3.74-3.57 (m, 2H).

Preparation of (4): To a solution of 3 (22.0 g, 96.9 mmol) in pyridine (250.0 mL), TrtCl (30.7 g, 110.5 mmol) was added. The mixture was stirred at 25° C. for 24 h. TLC showed 3 was consumed completely, H₂O was added to the mixture. Then filtered and the filtrate diluted with EA, the organic layer was washed with NaHCO₃ and brine. The solvent was concentrated under reduced pressure and then purified by c.c. (PE/EA=5:1˜0:1) to give 4 (38.8 g, 82.5 mmol, 85.1% yield) as a white solid. ESI-LCMS: m/z 470.1 [M+H]⁺.

Preparation of (5): To a solution of 4 (38.8 g, 82.5 mmol) in DMF (500.0 mL), collidine (10.0 g, 107.3 mmol), TrtCl (27.6 g, 99.1 mmol) were added followed by AgNO₃ (18.0 g, 105.1 mmol). The mixture was stirred at 25° C. for 4 h. TLC showed 4 was consumed completely. Then filtered and the filtrate diluted with EA. The organic layer was washed with NaHCO₃ and brine. The solvent was concentrated under reduced pressure and then purified by c.c. (PE/EA=5:1 ˜1:1) to give a mixture of 5 (52.3 g, 73.5 mmol, 86.3% yield) as white solid. ESI-LCMS: m/z 711.1 [M+H]⁺.

Preparation of (6): To a solution of 5 (52.3 g, 73.5 mmol) in DCM (500.0 mL), DMAP (8.9 g, 73.5 mmol), TEA (14.9 g, 147.3 mmol, 20.6 mL) were added, cool down to 0° C., TfCl (16.1 g, 95.6 mmol) dissolved in DCM (100.0 mL) were dropped. The mixture was stirred at 25° C. for 1 h. TLC showed 5 was consumed completely. Then filtered and the solution diluted with EA. The organic layer was washed with NaHCO₃ and brine. The solvent was concentrated under reduced pressure to get crude 6 (60.2 g) as a brown solid. ESI-LCMS: m/z 844.2 [M+H]⁺.

Preparation of (7): To a solution of 6 (60.2 g) in DMF (500.0 mL), KOAc (36.1 g, 367.8 mmol) were added, The mixture was stirred at 25° C. for 14 h and 50° C. for 3 h, TLC showed 6 was consumed completely. Then filtered and the solution diluted with EA. The organic layer was washed with H₂O and brine. The solvent was concentrated under reduced pressure, residue was purified by c.c. (PE/EA=5:1 ˜1:1) to give 7 (28.0 g, 39.3 mmol, 53.5% yield) as yellow solid. ESI-LCMS: m/z 710.2 [M−H]⁻; ¹H-NMR (400 MHz, DMSO-d₆): δ 7.37-7.25 (m, 33H), 6.34-6.31 (m, 2H), 6.13-6.10 (m, 1H), 5.08 (d, J=4.2 Hz, 1H), 3.99 (d, J=7.6 Hz, 1H), 3.74 (s, 1H), 3.12 (t, J=9.2 Hz, 1H), 2.72-2.69 (m, 1H).

Preparation of (8): To a solution of 7 (28.0 g, 39.3 mmol) in DCM (300.0 mL), DAST (31.6 g, 196.6 mmol) was added at 0° C., the mixture was stirred at 25° C. for 16 h, TLC showed 7 was consumed completely. Then the solution was diluted with EA, washed with NaHCO₃ twice, the solvent was removed under reduced pressure, residue was purified by c.c. (PE/EA=5:1 ˜3:1) to give 8 (5.0 g, 7.0 mmol, 17.8% yield) as a white solid. ESI-LCMS: m/z 748.2 [M+2NH₄]⁺; ¹H-NMR (400 MHz, DMSO-d₆): δ 7.57-7.18 (m, 35H), 6.30 (d, J=8.8 Hz, 1H), 6.00 (d, J=19.5 Hz, 1H), 5.92-5.88 (m, 1H), 4.22-4.17 (m, 2H), 3.94 (s, 0.5H), 3.80 (s, 0.5H), 3.35-3.31 (m, 1H), 3.14-3.10 (m, 1H); ¹⁹F-NMR (376 MHz, DMSO-d₆): δ −193.54.

Preparation of (9): To a solution of 8 (5.0 g, 7.0 mmol) in DCM (60.0 mL) was added DCA (3.6 mL) and TES (15.0 mL). The mixture was stirred at 20° C. for 1 h, TLC showed 8 was consumed completely. Then the solution was concentrated under reduced pressure, the residue was purified by Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=0/1; Detector, UV 254 nm. This resulted in to give 9 (1.6 g, 6.9 mmol, 98.5% yield) as a white solid. ESI-LCMS: m/z 229.9 [M+H]⁺; ¹H-NMR (400 MHz, DMSO-d₆): δ 8.06-8.04 (m, 1H), 7.48-7.43 (m, 1H), 6.39 (d, J=9.0 Hz, 1H), 6.31-6.27 (m, 1H), 6.16-6.11 (m, 1H), 5.63 (s, 1H), 5.26 (s, 1H), 4.95-4.81 (m, 1H), 4.20-411 (m, 1H), 3.95 (d, J=8.2 Hz, 1H), 3.84 (d, J=12.4 Hz, 1H), 3.64 (d, J=12.1 Hz, 1H); ¹⁹F-NMR (376 MHz, DMSO-d₆): δ −201.00.

Preparation of (10): To a solution of 9 (1.6 g, 6.9 mmol) in pyridine (20.0 mL) was added DMTrCl (3.5 g, 10.5 mmol) at 20° C. and stirred for 1 h. TLC showed 9 was consumed completely. Water was added and extracted with EA, the organic layer was washed with NaHCO₃ and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O (0.5% NH₄HCO₃)=1/1; Detector, UV 254 nm. This resulted in to give 10 (2.2 g, 4.2 mmol, 60.8% yield) as a white solid. ESI-LCMS: m/z 530.1 [M−H]⁻; ¹H-NMR (400 MHz, DMSO-d₆): δ 7.93-7.91 (m, 1H), 7.47-7.23 (m, 10H), 6.91-6.89 (m, 4H), 6.41 (d, J=8.8 Hz, 1H), 6.13 (d, J=18.8 Hz, 1H), 6.00-5.96 (m, 1H), 5.68 (d, J=6.6 Hz, 1H), 5.01 (d, J=4.2 Hz, 0.5H), 4.88 (d, J=4.2 Hz, 0.5H), 4.42-4.31 (m, 1H), 4.10-4.08 (m, 1H), 3.74 (s, 6H), 3.40-3.34 (m, 2H); ¹⁹F-NMR (376 MHz, DMSO-d₆): δ −199.49.

Preparation of Example 34 monomer: To a solution of 10 (2.2 g, 4.2 mmol) in DCM (20.0 mL) was added DCI (415 mg, 3.5 mmol) and CEP (1.5 g, 4.9 mmol) under N₂ pro. The mixture was stirred at 20° C. for 0.5 h. TLC showed 10 was consumed completely. The product was extracted with DCM, the organic layer was washed with H₂O and brine. Then the solution was concentrated under reduced pressure and the residue was purified by cc (PE/EA=5:1 ˜ 1:1) and Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in to give Example 34 monomer (2.1 g, 3.0 mmol, 73.1% yield) as a white solid. ESI-ESI-LCMS: m/z 732.2 [M+H]⁺; ¹H-NMR (400 MHz, DMSO-d₆): δ 7.98-7.92 (m, 1H), 7.42-7.24 (m, 10H), 6.91-6.85 (m, 4H), 6.43-6.39 (m, 1H), 6.18-6.11 (m, 1H), 6.01-5.97 (m, 1H), 5.22-5.19 (m, 0.5H), 5.09-5.06 (m, 0.5H), 4.73-4.52 (m, 1H), 4.21-4.19 (m, 1H), 3.79-3.62 (m, 7H), 3.57-3.47 (m, 4H), 3.32-3.28 (m, 1H), 2.75-2.58 (m, 1H), 1.13-0.92 (m, 12H); ¹⁹F-NMR (376 MHz, DMSO-d₆): δ −196.82, −196.84, −197.86-197.88; ³¹P-NMR (162 MHz, DMSO-d₆): δ 149.88, 149.83, 149.39, 149.35.

### Example 35. Synthesis of Monomer

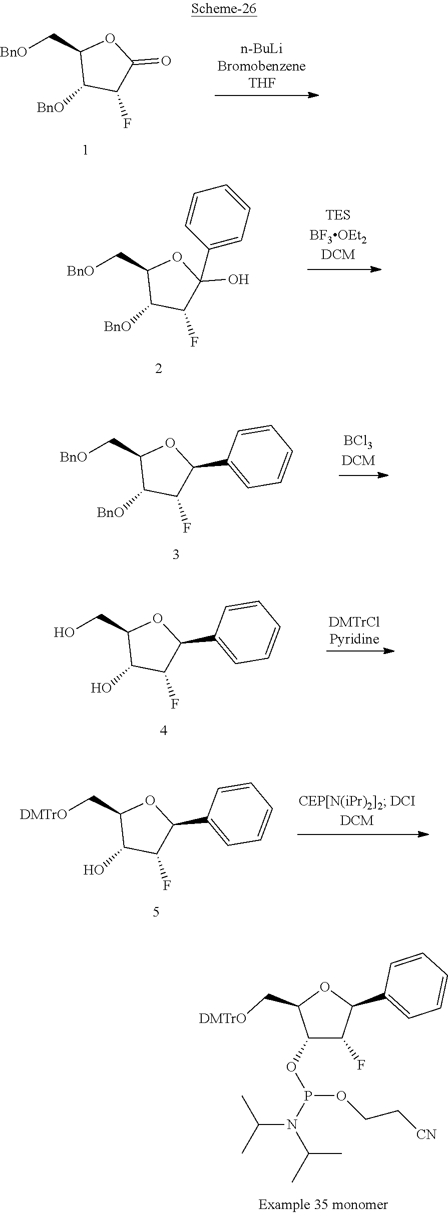

Preparation of (2): To the solution of Bromobenzene (2.1 g, 13.6 mmol) in dry THF (15 mL) was added 1.6 M n-BuLi (7 mL, 11.8 mmol) drop wise at −78° C. The mixture was stirred at −78° C. for 0.5 h. Then the 1 (3.0 g, 9.1 mmol, Wang, Guangyi et al, Journal of Medicinal Chemistry, 2016, 59 (10), 4611-4624) was dissolved in THF (15 mL) and added to the mixture drop wise with keeping at −78° C. Then the reaction mixture was stirred at −78° C. for 1 hr. LC-MS showed 1 was consumed completely. Then the solution was added to saturated aq. NH₄Cl and the resulting mixture was extracted with EA. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 2 (3.0 g, 7.3 mmol, 80.0%) as a white solid. ESI-LCMS: m/z 391 [M−OH]⁻.

Preparation of (3): To the solution of 2 (4.0 g, 9.8 mmol) in DCM (40 mL) was added TES (1.9 g, 11.7 mmol) at −78° C., and the mixture was added BF₃·OEt₂ (2.1 g, 14.7 mmol) drop wise at −78° C. The mixture was stirred at −40° C. for 1 hr. LC-MS showed 2 was consumed completely. Then the solution was added to saturated aq. NaHCO₃ and the resulting mixture was extracted with DCM. The combined organic layer was washed with water and brine, dried over Na₂SO₄, and concentrated under reduced pressure to give a residue which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in to give 3 (3.1 g, 5.3 mmol, 54.0%) as a water clear oil. ESI-LCMS: m/z 410 [M+H₂O]⁺; ¹H-NMR (400 MHz, CDCl₃: δ 7.48-7.25 (m, 15H), 5.24-5.13 (m, 1H), 4.93-4.74 (m, 1H), 4.74-4.46 (m, 4H), 4.37-4.25 (m, 1H), 4.19-4.05 (m, 1H), 4.00-3.80 (m, 1H), 3.77-3.63 (m, 1H). ¹⁹F-NMR (376 MHz, CDCl₃): δ −196.84.

Preparation of (4): To the solution of 3 (2.1 g, 5.3 mmol) in dry DCM (20 mL) was added 1 M BCl₃ (25 mL, 25.5 mmol) drop wise at −78° C., and the reaction mixture was stirred at −78° C. for 0.5 hr. LC-MS showed 3 was consumed completely. After completion of reaction, the resulting mixture was poured into water (50 mL). The solution was extracted with DCM and the combined organic layer was concentrated under reduced pressure to give a crude. The crude in MeOH (4 mL) was added 1 M NaOH (15 mL), and the mixture was stirred at r.t for 5˜10 min. The mixture was extracted with EA. The combined organic layer was washed with brine, dried over Na₂SO₄, and concentrated under reduced pressure to give a residue which was purified by silica gel column chromatography (eluent, DCM:MeOH=40:1˜15:1) to give 4 (1.0 g, 4.7 mmol, 88.6%) as a water clear oil. ESI-LCMS: m/z 211 [M−H]⁻; ¹H-NMR (400 MHz, DMSO-d₆): δ 7.58-7.19 (m, 5H), 5.41 (d, J=6.1 Hz, 1H), 5.09-5.95 (m, 1H), 5.95-4.84 (m, 1H), 4.82-4.59 (m, 1H), 4.14-3.94 (m, 1H), 3.89-3.80 (m, 1H), 3.78-3.67 (m, 1H), 3.65-3.53 (m, 1H). ¹⁹F-NMR (376 MHz, DMSO-d₆): δ −196.46.

Preparation of (5): To a solution of 4 (1.0 g, 4.7 mmol) in Pyridine (10 mL) was added DMTrCl (2.0 g, 5.7 mmol). The reaction mixture was stirred at r.t. for 2 hr. LCMS showed 4 was consumed and water (100 mL) was added. The product was extracted with EA (100 mL) and the organic layer was washed with brine and dried over Na₂SO₄ and concentrated to give the crude. The crude was further purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. This resulted in to give 5 (2.1 g, 4.1 mmol, 87.0%) as a red oil. ESI-LCMS: m/z 513 [M−H]⁻; ¹H-NMR (400 MHz, DMSO-d₆): δ 7.56-7.16 (m, 14H), 6.94-9.80 (m, 4H), 5.45 (d, J=6.3 Hz, 1H), 5.21-5.09 (m, 1H), 4.89-4.68 (m, 1H), 4.18-4.03 (m, 2H), 3.74 (s, 6H), 3.33-3.29 (m, 1H), 3.26-3.17 (m, 1H). ¹⁹F-NMR (376 MHz, DMSO-d₆): δ −194.08.

Preparation of Example 35 monomer: To a suspension of 5 (2.1 g, 4.1 mmol) in DCM (20 mL) was added DCI (410 mg, 3.4 mmol) and CEP[N(iPr)₂]₂ (1.5 g, 4.9 mmol). The mixture was stirred at r.t. for 1 h. LC-MS showed 5 was consumed completely. The solution was washed with water twice and washed with brine and dried over Na₂SO₄. Then concentrated to give the crude. The crude was purification by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give Example 35 monomer (2.1 g, 2.9 mmol, 70.0%) as a white solid. ESI-LCMS: m/z 715 [M+H]⁺; ¹H-NMR (400 MHz, DMSO-d₆): δ 7.59-7.16 (m, 14H), 6.94-9.80 (m, 4H), 5.26-5.12 (m, 1H), 5.06-4.77 (m, 1H), 4.50-4.20 (m, 1H), 4.20-4.10 (m, 1H), 3.83-3.63 (m, 7H), 3.59-3.37 (m, 4H), 3.25-3.13 (m, 1H), 2.80-2.66 (m, 1H), 2.63-2.53 (m, 1H), 1.18-0.78 (m, 12H). ¹⁹F-NMR (376 MHz, DMSO-d₆): δ −194.40, −194.42, −194.50, −194.53. ³¹P-NMR (162 MHz, DMSO-d₆): δ 149.38, 149.30, 149.02, 148.98.

### Example 36: Synthesis of 5′ End Cap Monomer

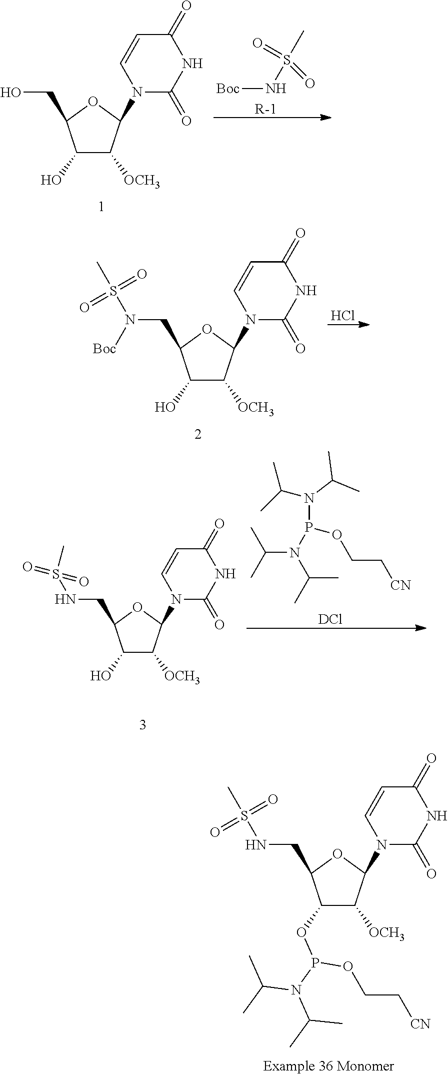

Preparation of (2): 1 (15 g, 58.09 mmol) and tert-butyl N-methylsulfonylcarbamate (17.01 g, 87.13 mmol) were dissolved in THF (250 mL), and PPh₃ (30.47 g, 116.18 mmol) was added followed by dropwise addition of DIAD (23.49 g, 116.18 mmol, 22.59 mL) at 0° C. The reaction mixture was stirred at 15° C. for 12 h. Upon completion as monitored by TLC (DCM/MeOH=10/1), the reaction mixture was evaporated to give a residue. The residue was purified by flash silica gel chromatography (ISCO®; 120 g SepaFlash® Silica Flash Column, Eluent of 0˜20% MeOH/DCM gradient @ 60 mL/min) to give 2 (6.9 g, 24.28% yield) as a white solid. ESI-LCMS: m/z 457.9 [M+Na]⁺; ¹H NMR (400 MHz, CDCl₃) δ=8.64 (br s, 1H), 7.64 (d, J=8.2 Hz, 1H), 5.88 (d, J=1.9 Hz, 1H), 5.80 (dd, J=2.2, 8.2 Hz, 1H), 4.19-4.01 (m, 3H), 3.90 (dt, J=5.5, 8.2 Hz, 1H), 3.82-3.78 (m, 1H), 3.64 (s, 3H), 3.32 (s, 3H), 2.75 (d, J=8.9 Hz, 1H), 1.56 (s, 9H).

Preparation of (3): 2 (6.9 g, 15.85 mmol) was dissolved in MeOH (40 mL), and a solution of HCl/MeOH (4 M, 7.92 mL) was added dropwise. The reaction mixture was stirred at 15° C. for 12 h, and then evaporated to give a residue. The residue was purified by flash silica gel chromatography (ISCO®; 40 g SepaFlash® Silica Flash Column, Eluent of 0˜10% MeOH/DCM gradient @ 40 mL/min) to give 3 (2.7 g, 50.30% yield) as a white solid. ESI-LCMS: m/z 336.0 [M+H]⁺; ¹H NMR (400 MHz, CD₃CN) δ=9.20 (br s, 1H), 7.52 (d, J=8.1 Hz, 1H), 5.75 (d, J=3.8 Hz, 1H), 5.64 (dd, J=2.0, 8.1 Hz, 1H), 5.60-5.52 (m, 1H), 4.15-3.99 (m, 1H), 3.96-3.81 (m, 2H), 3.46 (s, 3H), 3.44-3.35 (m, 1H), 3.34-3.26 (m, 1H), 2.92 (s, 3H).

Preparation of (Example 36 monomer): To a solution of 3 (2.14 g, 6.38 mmol) in DCM (20 mL) was added dropwise 3-bis(diisopropylamino)phosphanyloxypropanenitrile (2.50 g, 8.30 mmol, 2.63 mL) at 0° C., followed by 1H-imidazole-4, 5-dicarbonitrile (829 mg, 7.02 mmol), and the mixture was purged under Ar for 3 times. The reaction mixture was stirred at 15° C. for 2 h. Upon completion, the mixture was quenched with 5% NaHCO₃ (20 mL), extracted with DCM (20 mL*2), washed with brine (15 mL), dried over Na₂SO₄, filtered, and evaporated to give a residue. The residue was purified by flash silica gel chromatography (ISCO®; 40 g SepaFlash® Silica Flash Column, Eluent of 0˜10% (Phase B: i-PrOH/DCM=1/2)/Phase A: DCM with 5% TEA gradient @ 40 mL/min) to give Example 36 monomer (1.73 g, 48.59% yield) as a white solid. ESI-LCMS: m/z 536.3 [M+H]⁺; ¹H NMR (400 MHz, CD₃CN) δ=7.58-7.48 (m, 1H), 5.83-5.78 (m, 1H), 5.71-5.64 (m, 1H), 4.40-4.29 (m, 1H), 4.19-4.07 (m, 1H), 3.98 (td, J=5.3, 13.3 Hz, 1H), 3.90-3.78 (m, 2H), 3.73-3.59 (m, 3H), 3.41 (d, J=14.8 Hz, 4H), 2.92 (br d, J=7.0 Hz, 3H), 2.73-2.63 (m, 2H), 1.23-1.11 (m, 12H); ³¹P NMR (162 MHz, CD₃CN) δ=149.81, 150.37.

### Example 37: Synthesis of 5′ End Cap Monomer

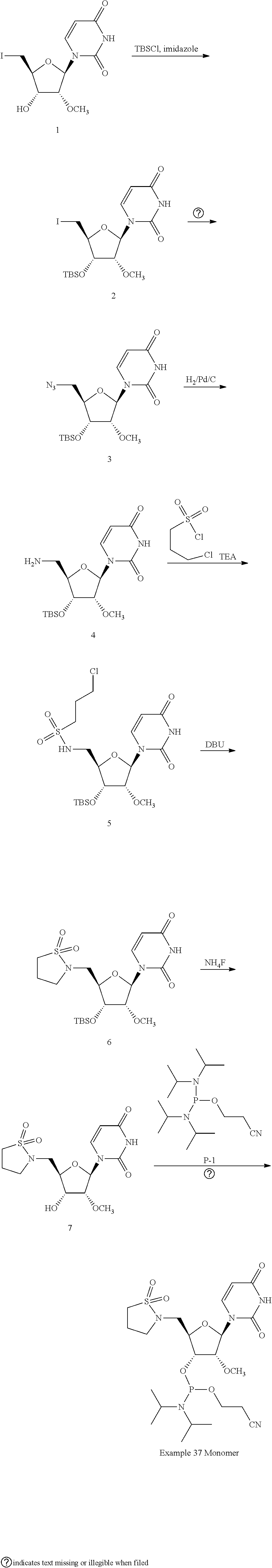

Preparation of (2): To a solution of 1 (10 g, 27.16 mmol) in DMF (23 mL) were added imidazole (3.70 g, 54.33 mmol) and TBSCl (8.19 g, 54.33 mmol) at 25° C. The mixture was stirred at 25° C. for 2 hr. Upon completion, the reaction mixture was diluted with H₂O (20 mL) and extracted with EA (30 mL*2). The combined organic layers were washed with brine (20 mL*2), dried over Na₂SO₄, filtered and concentrated under reduced pressure to give 2 (13 g, 99.2% yield) as a white solid. ESI-LCMS: m/z 482.9 [M+H]⁺.

Preparation of (3): To a solution of 2 (35.00 g, 72.56 mmol) in DMF (200 mL) was added NaN₃ (14.15 g, 217.67 mmol). The mixture was stirred at 60° C. for 17 h. Upon completion, the reaction mixture was diluted with H₂O (200 mL) and extracted with EA (200 mL*2). The combined organic layers were washed with brine (100 mL*2), dried over Na₂SO₄, filtered and concentrated under reduced pressure to give 3 (31.8 g, crude) as a yellow solid. ESI-LCMS: m/z 398.1 [M+H]⁺; ¹H NMR (400 MHz, DMSO-d₆) δ=11.21 (d, J=1.3 Hz, 1H), 7.50 (d, J=8.1 Hz, 1H), 5.57 (d, J=4.5 Hz, 1H), 5.46 (dd, J=2.1, 8.0 Hz, 1H), 4.06 (t, J=5.2 Hz, 1H), 3.81-3.64 (m, 2H), 3.44-3.30 (m, 2H), 2.31-2.25 (m, 3H), 0.65 (s, 9H), −0.13 (s, 6H).

Preparation of (4): To a solution of 3 (7 g, 17.61 mmol) in THF (60 mL) was added Pd/C (2 g) at 25° C. The reaction mixture was stirred at 25° C. for 3 h under H₂ atmosphere (15 PSI). The reaction mixture was filtered, and the filtrate was concentrated to give 4 (5.4 g, 75.11% yield) as a gray solid. ESI-LCMS: m/z 372.1 [M+H]⁺; ¹H NMR (400 MHz, DMSO-d₆) δ=7.93 (d, J=8.0 Hz, 1H), 5.81 (d, J=5.5 Hz, 1H), 5.65 (d, J=8.3 Hz, 1H), 4.28 (t, J=4.6 Hz, 1H), 3.88 (t, J=5.3 Hz, 1H), 3.74 (q, J=4.6 Hz, 1H), 3.31 (s, 3H), 2.83-2.66 (m, 2H), 0.88 (s, 9H), 0.09 (s, 6H).

Preparation of (5): To a solution of 4 (3 g, 8.08 mmol) in DCM (30 mL) was added TEA (2.45 g, 24.23 mmol, 3.37 mL) followed by dropwise addition of 3-chloropropane-1-sulfonyl chloride (1.50 g, 8.48 mmol, 1.03 mL) at 25° C. The reaction mixture was stirred at 25° C. for 18 h under N₂ atmosphere. Upon completion, the reaction mixture was diluted with H₂O (50 mL) and extracted with DCM (50 mL*2). The combined organic layers were washed with brine (50 mL*2), dried over Na₂SO₄, filtered and concentrated under reduced pressure. The residue was purified by flash silica gel chromatography (ISCO®; 24 g SepaFlash® Silica Flash Column, Eluent of 0˜30% MeOH/DCM @ 50 mL/min) to give 5 (3.6 g, 84.44% yield) as a white solid. ESI-LCMS: m/z 512.1 [M+H]⁺; ¹H NMR (400 MHz, DMSO-d₆) δ=11.42 (s, 1H), 7.75 (d, J=8.1 Hz, 1H), 7.49 (t, J=6.2 Hz, 1H), 5.83 (d, J=5.8 Hz, 1H), 5.70-5.61 (m, 1H), 4.33-4.23 (m, 1H), 3.95 (t, J=5.5 Hz, 1H), 3.90-3.78 (m, 1H), 3.73 (t, J=6.5 Hz, 2H), 3.30 (s, 3H), 3.26-3.12 (m, 4H), 2.14-2.02 (m, 2H), 0.88 (s, 9H), 0.11 (d, J=3.3 Hz, 6H).

Preparation of (6): To a solution of 5 (5 g, 9.76 mmol) in DMF (45 mL) was added DBU (7.43 g, 48.82 mmol, 7.36 mL). The mixture was stirred at 25° C. for 16 h. The reaction mixture was concentrated to give a residue, diluted with H₂O (50 mL) and extracted with EA (50 mL*2). The combined organic layers were washed with brine (50 mL*2), dried over Na₂SO₄, filtered and concentrated under reduced pressure. The residue was purified by flash silica gel chromatography (ISCO®; 24 g SepaFlash® Silica Flash Column, Eluent of 0˜80% EA/PE @ 40 mL/min) to give 6 (4.4 g, 89.06% yield) as a white solid. ESI-LCMS: m/z 476.1 [M+H]⁺; ¹H NMR (400 MHz, DMSO-d₆) δ=11.43 (d, J=1.7 Hz, 1H), 7.72 (d, J=8.1 Hz, 1H), 5.82 (d, J=4.8 Hz, 1H), 5.67 (dd, J=2.1, 8.1 Hz, 1H), 4.22 (t, J=5.1 Hz, 1H), 3.99-3.87 (m, 2H), 3.33-3.27 (m, 6H), 3.09 (dd, J=6.6, 14.7 Hz, 1H), 2.26-2.16 (m, 2H), 0.88 (s, 9H), 0.10 (d, J=3.8 Hz, 6H).

Preparation of (7): To a solution of 6 (200 mg, 420.49 umol) in MeOH (2 mL) was added NH₄F (311.48 mg, 8.41 mmol, 20 eq), and the mixture was stirred at 80° C. for 2 h. The mixture was filtered and concentrated to give a residue, which was purified by flash silica gel chromatography (ISCO®; 4 g SepaFlash® Silica Flash Column, Eluent of 0˜50% MeOH/DCM @ 20 mL/min) to give 7 (120 mg, 76.60% yield) as a white solid. ESI-LCMS: m/z 362.1 [M+H]⁺; ¹H NMR (400 MHz, DMSO-d₆) δ=11.37 (br s, 1H), 7.68 (d, J=8.1 Hz, 1H), 5.81 (d, J=4.6 Hz, 1H), 5.65 (d, J=8.0 Hz, 1H), 4.02 (q, J=5.6 Hz, 1H), 3.95-3.83 (m, 2H), 3.34 (s, 9H), 3.09 (dd, J=6.9, 14.6 Hz, 1H), 2.26-2.14 (m, 2H).

Preparation of (Example 37 monomer): To a solution of 7 (1.5 g, 4.15 mmol) in CH₃CN (12 mL) were added 3-bis(diisopropylamino)phosphanyloxypropanenitrile (1.63 g, 5.40 mmol, 1.71 mL) and 1H-imidazole-4,5-dicarbonitrile (539.22 mg, 4.57 mmol) in one portion at 0° C. The reaction mixture was gradually warmed to 25° C. The reaction mixture was stirred at 25° C. for 2 h under N₂ atmosphere. Upon completion, the reaction mixture was diluted with NaHCO₃ (20 mL) and extracted with DCM (20 mL*2). The combined organic layers were washed with brine (20 mL*2), dried over Na₂SO₄, filtered and concentrated under reduced pressure to give a residue, which was purified by flash silica gel chromatography (ISCO®; 12 g SepaFlash® Silica Flash Column, Eluent of 0˜85% EA/PE with 0.5% TEA @ 30 mL/min to give Example 37 monomer (800 mg, 33.6% yield) as a white solid. ESI-LCMS: m/z 562.3 [M+H]⁺; ¹H NMR (400 MHz, CD₃CN) δ=9.28 (br s, 1H), 7.55 (br dd, J=8.3, 12.8 Hz, 1H), 5.86 (br d, J=3.9 Hz, 1H), 5.65 (br d, J=8.0 Hz, 1H), 4.33-4.06 (m, 2H), 4.00-3.89 (m, 1H), 4.08-3.86 (m, 1H), 3.89-3.72 (m, 4H), 3.43 (br d, J=15.1 Hz, 6H), 3.23-3.05 (m, 3H), 2.69 (br s, 2H), 2.36-2.24 (m, 2H), 1.26-1.10 (m, 12H): ³¹P NMR (162 MHz, CD₃CN) δ=149.94, 149.88.

### Example 38: Synthesis of 5′ End Cap Monomer

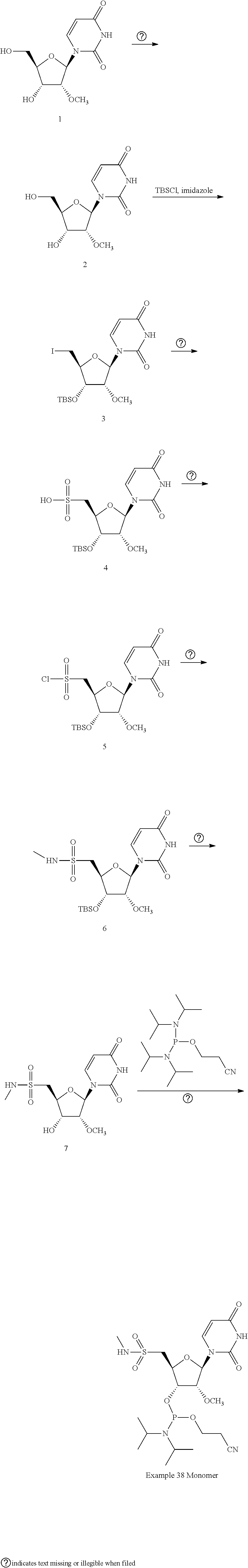

Preparation of (2): To a solution of 1 (30 g, 101.07 mmol, 87% purity) in CH₃CN (1.2 L) and Py (60 mL) were added I₂ (33.35 g, 131.40 mmol, 26.47 mL) and PPh₃ (37.11 g, 141.50 mmol) in one portion at 10° C. The reaction was stirred at 25° C. for another 48 h. The mixture was diluted with aq. Na₂S203 (300 mL) and aq·NaHCO₃ (300 mL), concentrated to remove CH₃CN, and then extracted with EtOAc (300 mL*3). The combined organic layers were washed with brine (300 mL), dried over Na₂SO₄, filtered and concentrated under reduced pressure to give a residue. The residue was purified by flash silica gel chromatography (ISCO®; 330 g SepaFlash® Silica Flash Column, Eluent of 0-60% Methanol/Dichloromethane gradient @ 100 mL/min) to give 2 (28.2 g, 72.00% yield, 95% purity) as a brown solid. ESI-LCMS: m/z 369.1 [M+H]⁺; ¹H NMR (400 MHz, DMSO-d₆) δ=11.43 (s, 1H), 7.68 (d, J=8.1 Hz, 1H), 5.86 (d, J=5.5 Hz, 1H), 5.69 (d, J=8.1 Hz, 1H), 5.46 (d, J=6.0 Hz, 1H), 4.08-3.96 (m, 2H), 3.90-3.81 (m, 1H), 3.60-3.51 (m, 1H), 3.40 (dd, J=6.9, 10.6 Hz, 1H), 3.34 (s, 3H).

Preparation of (3): To a solution of 2 in DMF (90 mL) were added imidazole (4.25 g, 62.48 mmol) and TBSCl (6.96 g, 46.18 mmol) in one portion at 15° C. The mixture was stirred at 15° C. for 6 h. The reaction mixture was quenched by addition of H₂O (300 mL) and extracted with EtOAc (300 mL*2). The combined organic layers were washed with brine (300 mL), dried over Na₂SO₄, filtered and concentrated under reduced pressure to give 3 (13.10 g, crude) as a white solid. ESI-LCMS: m/z 483.0 [M+H]⁺.

Preparation of (4): To a solution of 3 (10 g, 20.73 mmol) in MeOH (20 mL), H₂O (80 mL), and dioxane (20 mL) was added Na₂SO₃ (15.68 g, 124.38 mmol), and the mixture was stirred at 80° C. for 24 h. The reaction mixture was concentrated under reduced pressure to remove MeOH. The aqueous layer was extracted with EtOAc (80 mL*2) and concentrated under reduced pressure to give a residue. The residue was triturated with MeOH (100*3 mL) to give 4 (9.5 g, 94.48% yield, 90% purity) as a white solid. ESI-LCMS: m/z 437.0 [M+H]⁺.

Preparation of (5): To a solution of 4 (11 g, 21.42 mmol, 85% purity) in DCM (120 mL) was added DMF (469.65 mg, 6.43 mmol, 494.37 uL) at 0° C., followed by dropwise addition of oxalyl dichloride (13.59 g, 107.10 mmol, 9.37 mL). The mixture was stirred at 20° C. for 2 h. The reaction mixture was quenched by addition of water (60 mL) and the organic layer 5 (0.1125 M, 240 mL DCM) was used directly for next step. (This reaction was set up for two batches and combined) ESI-LCMS: m/z 455.0 [M+H]⁺.

Preparation of (6): 5 (186.4 mL, 0.1125 M in DCM) was diluted with DCM (60 mL) and treated with methylamine (3.26 g, 41.93 mmol, 40% purity). The mixture was stirred at 20° C. for 2 h. The reaction mixture was concentrated under reduced pressure to give a residue. The residue was purified by flash silica gel chromatography (ISCO®; 40 g SepaFlash® Silica Flash Column, Eluent of 0˜10%, MeOH/DCM gradient @ 40 mL/min) to give AGS-9-3-008 (1.82 g, 18.53% yield, 96% purity) as a yellow solid. ESI-LCMS: m/z 472.0 [M+Na]⁺; ¹H NMR (400 MHz, CDCl₃) δ=9.08 (s, 1H), 7.31 (d, J=8.1 Hz, 1H), 5.78 (d, J=8.1 Hz, 1H), 5.57 (d, J=3.8 Hz, 1H), 4.61-4.48 (m, 1H), 4.41-4.27 (m, 2H), 4.13-4.03 (m, 1H), 3.46 (s, 3H), 3.43-3.33 (m, 2H), 2.78 (d, J=5.2 Hz, 3H), 0.92 (s, 9H), 0.13 (s, 6H).

Preparation of (7): To a solution of 6 (2.3 g, 5.12 mmol) in MeOH (12 mL) was added HCl/MeOH (4 M, 6.39 mL). The mixture was stirred at 20° C. for 2 h. The reaction mixture was concentrated under reduced pressure to give a residue. The residue was purified by flash silica gel chromatography (ISCO®; 24 g SepaFlash® Silica Flash Column, Eluent of 0˜15%, MeOH/DCM gradient @ 30 mL/min) to give 7 (1.4 g, 79.98% yield) as a pink solid. ESI-LCMS: m/z 336.1 [M+H]⁺; ¹H NMR (400 MHz, CDCl₃) δ=9.12 (s, 1H), 7.39 (d, J=8.0 Hz, 1H), 5.79 (d, J=3.3 Hz, 1H), 5.66 (dd, J=2.1, 8.2 Hz, 1H), 5.13 (s, 1H), 4.13 (t, J=4.0, 7.4 Hz, 1H), 4.07-4.02 (m, 1H), 3.87 (dd, J=3.3, 5.5 Hz, 1H), 3.47 (s, 3H), 3.43-3.37 (m, 2H), 2.65 (d, J=4.5 Hz, 3H).

Preparation of (Example 38 monomer): To a mixture of 7 (1.7 g, 5.07 mmol) and 4A MS (1.4 g) in MeCN (18 mL) was added 3-bis(diisopropylamino)phosphanyloxypropanenitrile (1.99 g, 6.59 mmol, 2.09 mL) at 0° C., followed by addition of 1H-imidazole-4,5-dicarbonitrile (658.57 mg, 5.58 mmol) in one portion at 0° C. The mixture was stirred at 20° C. for 2 h. Upon completion, the reaction mixture was quenched by addition of sat. NaHCO₃ solution (20 mL) and diluted with DCM (40 mL). The organic layer was washed with sat. NaHCO₃ (20 mL*2), dried over Na₂SO₄, filtered and concentrated under reduced pressure to give a residue. The residue was purified by a flash silica gel column (0% to 5% i-PrOH in DCM with 5% TEA) to give Example 38 monomer (1.30 g, 46.68% yield) as a white solid. ESI-LCMS: m/z 536.2 [M+H]⁺; ¹H NMR (400 MHz, CD₃CN) 8=9.00 (s, 1H), 7.40 (d, J=8.0 Hz, 1H), 5.85-5.76 (m, 1H), 5.64 (d, J=8.0 Hz, 1H), 5.08 (d, J=5.0 Hz, 1H), 4.42-4.21 (m, 2H), 4.00 (td, J=4.6, 9.3 Hz, 1H), 3.89-3.61 (m, 4H), 3.47-3.40 (m, 4H), 3.37-3.22 (m, 1H), 2.71-2.60 (m, 5H), 1.21-1.16 (m, 11H), 1.21-1.16 (m, 1H); ³¹P NMR (162 MHz, CD₃CN) δ=150.07, 149.97

### Example 39: Synthesis of 5′ End Cap Monomer

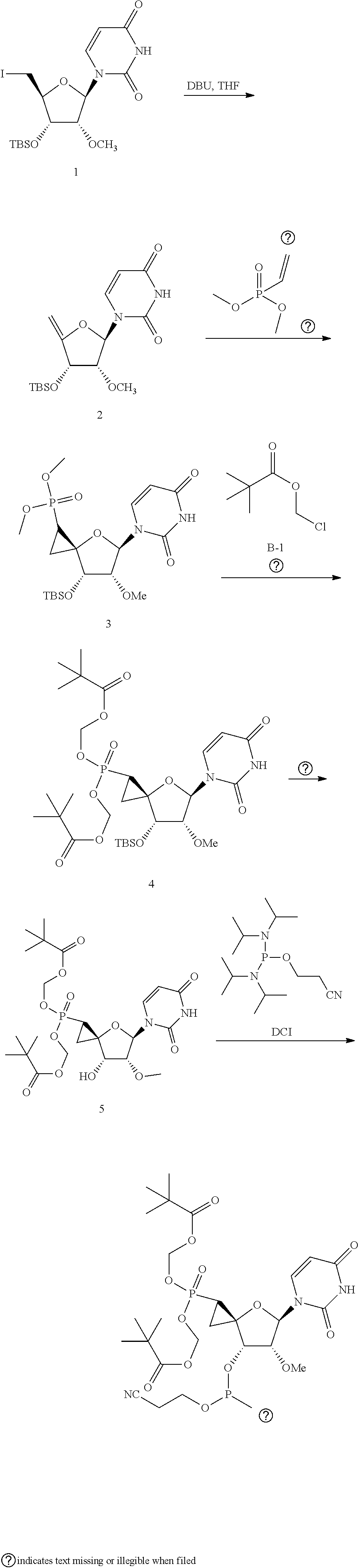

Preparation of (2): To a solution of 1 (13.10 g, 27.16 mmol) in THF (100 mL) was added DBU (20.67 g, 135.78 mmol, 20.47 mL). The mixture was stirred at 60° C. for 6 h. Upon completion, the reaction mixture was quenched by addition of sat. NH₄Cl solution (600 mL) and extracted with EA (600 mL*2). The combined organic layers were washed with brine (100 ml), dried over Na₂SO₄, filtered and concentrated under reduced pressure to give a residue. The residue was purified by flash silica gel chromatography (ISCO®; 120 g SepaFlash® Silica Flash Column, Eluent of 0˜50% (Phase B: ethyl acetate:dichloromethane=1:1)/Phase A: petroleum ethergradient@ 45 mL/min) to give 2 (5.9 g, 60.1% yield) as a white solid. ESI-LCMS: m/z 355.1 [M+H]⁺; ¹H NMR (400 MHz, DMSO-d₆) δ=11.61-11.30 (m, 1H), 7.76-7.51 (m, 1H), 6.04 (d, J=5.4 Hz, 1H), 5.75 (s, 1H), 5.73-5.67 (m, 1H), 4.78 (d, J=4.9 Hz, 1H), 4.41 (d, J=1.1 Hz, 1H), 4.30 (t, J=4.8 Hz, 1H), 4.22 (d, J=1.4 Hz, 1H), 4.13 (t, J=5.1 Hz, 1H), 4.06-3.97 (m, 1H), 3.94-3.89 (m, 1H), 3.82-3.75 (m, 1H), 3.33 (s, 3H), 3.30 (s, 2H), 1.17 (t, J=7.2 Hz, 1H), 0.89 (s, 9H), 0.16-0.09 (m, 6H).

Preparation of (3): To a solution of 2 (4 g, 11.28 mmol) in DCM (40 mL) was added Ru(II)-Pheox (214.12 mg, 338.53 umol) in one portion followed by addition of diazo (dimethoxyphosphoryl)methane (2.54 g, 16.93 mmol) dropwise at 0° C. under N₂. The reaction was stirred at 20° C. for 16 h. Upon completion, the reaction mixture was filtered and concentrated under reduced pressure to give a residue. The residue was purified by flash silica gel chromatography (ISCO®; 80 g SepaFlash® Silica Flash Column, Eluent of 0˜4% MeOH/DCM@ 60 mL/min) to give 3 (5 g, 86.47% yield) as a red liquid. ESI-LCMS: m/z 477.1 [M+H]⁺; ¹H NMR (400 MHz, DMSO-d₆) δ=11.46 (s, 1H), 7.49 (d, J=8.0 Hz, 1H), 6.01-5.87 (m, 1H), 5.75 (dd, J=2.0, 8.0 Hz, 1H), 4.58 (d, J=3.8 Hz, 1H), 4.23 (dd, J=3.8, 7.8 Hz, 1H), 3.80-3.68 (m, 6H), 3.30 (s, 3H), 1.65-1.46 (m, 2H), 1.28-1.16 (m, 1H), 0.91 (s, 9H), 0.10 (d, J=4.3 Hz, 6H); ³¹P NMR (162 MHz, DMSO-d₆) δ=27.5

Preparation of (4): To a mixture of 3 (2.8 g, 5.88 mmol) and NaI (1.76 g, 11.75 mmol) in CH₃CN (30 mL) was added chloromethyl 2,2-dimethylpropanoate (2.21 g, 14.69 mmol, 2.13 mL) at 25° C. The mixture was stirred at 80° C. for 40 h under Ar. The reaction mixture was filtered and concentrated under reduced pressure to give a residue. The residue was purified by flash silica gel chromatography (ISCO®; 40 g SepaFlash® Silica Flash Column, Eluent of 0˜50% Ethylacetate/Petroleum ether gradient @ 40 mL/min) to give 4 (2.1 g, 51.23% yield, 97% purity) as a yellow solid. ESI-LCMS: 677.3 [M+H]⁺.

Preparation of (5): A mixture of 4 (2.09 g, 3.09 mmol) in H₂O (1.5 mL) and HCOOH (741.81 mg, 15.44 mmol, 6 mL) was stirred at 15° C. for 40 h. Upon completion, the reaction mixture was quenched by saturated aq. NaHCO₃ (300 mL) and extracted with EA (300 mL*2). The combined organic layers were washed with brine (300 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜5% Methanol/Dichloromethane@ 45 mL/min) to give 5 (1.51 g, 85.19% yield) as a yellow solid. ESI-LCMS: 585.1 [M+Na]⁺; ¹H NMR (400 MHz, DMSO-d₆) δ=11.45 (d, J=1.8 Hz, 1H), 7.44 (d, J=8.2 Hz, 1H), 6.04 (d, J=7.5 Hz, 1H), 5.78-5.51 (m, 6H), 4.39 (t, J=4.4 Hz, 1H), 4.15 (dd, J=4.3, 7.4 Hz, 1H), 4.03 (q, J=7.1 Hz, 1H), 1.99 (s, 1H), 1.66 (dd, J=8.6, 10.8 Hz, 1H), 1.55-1.29 (m, 2H), 1.18 (d, J=2.0 Hz, 18H).

Preparation of (Example 39 monomer): To a solution of 5 (2.5 g, 4.44 mmol) in MeCN (30 mL) was added 3-bis(diisopropylamino)phosphanyloxypropanenitrile (1.74 g, 5.78 mmol, 1.84 mL) at 0° C., followed by 1H-imidazole-4,5-dicarbonitrile (577.36 mg, 4.89 mmol) in one portion under Ar. The mixture was gradually warmed to 20° C. and stirred at 20° C. for 1 h. The reaction mixture was quenched by addition of sat·NaHCO₃ solution (50 mL) and diluted with DCM (250 mL). The organic layer was washed with sat. NaHCO₃ solution (50 mL*2), dried over Na₂SO₄, filtered and concentrated under reduced pressure to give a residue. The residue was purified by a flash silica gel column (0% to 50% EA/PE with 0.5% TEA) to give Example 39 monomer (1.85 g, 54.1% yield) as a white solid. ESI-LCMS: 785.2 [M+Na]⁺; 1H NMR (400 MHz, CD₃CN) δ=9.18 (s, 1H), 7.31 (d, J=8.3 Hz, 1H), 6.06 (d, J=7.8 Hz, 1H), 5.72-5.60 (m, 5H), 4.85-4.76 (m, 1H), 4.27 (m, 1H), 3.93-3.64 (m, 4H), 3.41 (d, J=16.6 Hz, 3H), 2.80-2.62 (m, 2H), 1.76-1.49 (m, 3H), 1.23-1.19 (m, 30H); ³¹P NMR (162 MHz, CD₃CN) δ=150.66 (s), 150.30, 24.77, 24.66.

### Example 40: Synthesis of 5′ End Cap Monomer

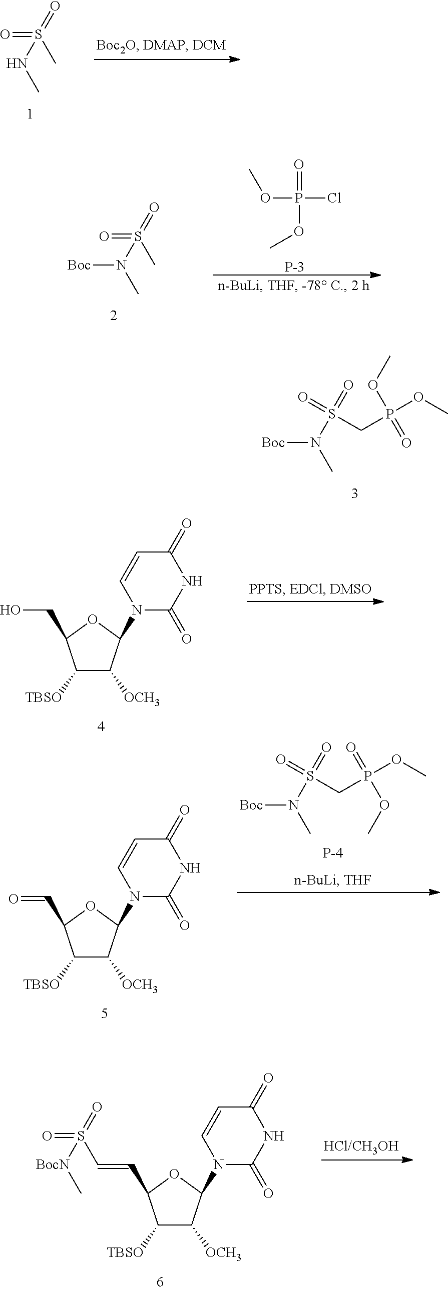

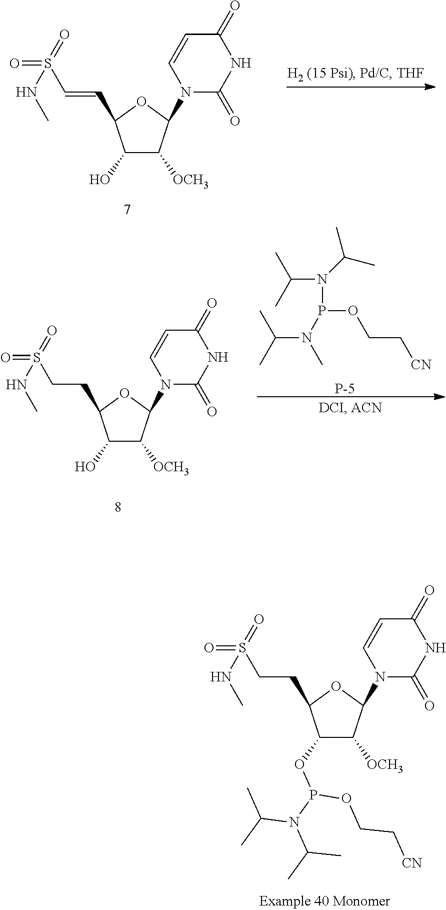

Preparation of (2): To a solution of 1 (15 g, 137.43 mmol) in DCM (75 mL) were added Boc₂O (31.49 g, 144.30 mmol, 33.15 mL) and DMAP (839.47 mg, 6.87 mmol, 0.05 eq) at 0° C. The mixture was stirred at 20° C. for 16 hr, and concentrated under reduced pressure to give 2 (29.9 g, crude) as a yellow oil. ¹H NMR (400 MHz, CDCl₃) δ=3.23 (s, 3H), 3.16 (s, 3H), 1.51 (s, 9H).

Preparation of (3): To a solution of 2 (24.9 g, 118.99 mmol) in THF (250 mL) was added n-BuLi (2.5 M, 47.60 mL) dropwise at −78° C. under Ar and stirred at −78° C. for 1 hr. P-3 (17.19 g, 118.99 mmol, 12.83 mL) was added at 0° C. and stirred for 1 hr. The reaction mixture was quenched by saturated aq. NH₄Cl (100 mL), and then extracted with EA (100 mL*2). The combined organic layers were washed with brine (100 mL*2), dried over Na₂SO₄, filtered and concentrated under reduced pressure to give a residue. The residue was purified by flash silica gel chromatography (ISCO®; 80 g SepaFlash® Silica Flash Column, Eluent of 0˜50% Ethylacetate/Petroleum ether gradient @ 65 mL/min) to give 3 (7.1 g, 18.62% yield) as a yellow oil. ESI-LCMS: 339.9 [M+Na]⁺; ¹H NMR (400 MHz, CDCl₃) δ=4.12 (s, 1H), 4.08 (s, 1H), 3.83 (s, 3H), 3.81 (s, 3H), 3.22 (s, 3H), 1.51 (s, 9H).

Preparation of (5): To a mixture of 4 (15 g, 40.27 mmol) and PPTS (10.12 g, 40.27 mmol) in DMSO (75 mL) was added EDCI (23.16 g, 120.81 mmol) at 20° C. The mixture was stirred at 20° C. for 4 hr. The reaction mixture was diluted with water (150 mL) and extracted with EA (150 mL*2). The combined organic layers were washed with brine (150 mL*2), dried over Na₂SO₄, filtered and concentrated under reduced pressure to give 5 (12 g, crude) as a white solid. ESI-LCMS: 371.2 [M+H]⁺; ¹H NMR (400 MHz, CDCl₃) δ=9.77 (s, 1H), 7.62 (d, J=8.1 Hz, 1H), 5.83-5.76 (m, 2H), 4.53 (d, J=4.3 Hz, 1H), 4.43 (br t, J=4.4 Hz, 1H), 3.95 (br t, J=4.7 Hz, 1H), 3.47-3.35 (m, 5H), 0.92 (s, 9H), 0.13 (d, J=5.8 Hz, 6H).

Preparation of (6): To a solution of P4 (8.02 g, 25.27 mmol) in THF (40 mL) was added n-BuLi (2.5 M, 8.42 mL) dropwise under Ar at −78° C., and the mixture was stirred at −78° C. for 0.5 hr. A solution of 4 (7.8 g, 21.05 mmol) in THF (40 mL) was added dropwise. The mixture was allowed to warm to 0° C. and stirred for another 2 hr. The reaction mixture was quenched by saturated aq. NH₄Cl solution (80 mL) and extracted with EA (80 mL). The combined organic layers were washed with brine (80 mL*2), dried over Na₂SO₄, filtered and concentrated under reduced pressure to give a residue. The residue was purified by flash silica gel chromatography (ISCO®; 80 g SepaFlash® Silica Flash Column, Eluent of 0˜38% ethylacetate/petroleum ether gradient @ 60 mL/min) to give 7 (7.7 g, 13.43 mmol, 63.8% yield) as a white solid. ESI-LCMS: 506.2 [M−tBu]⁺; ¹H NMR (400 MHz, CDCl₃) δ=8.97 (s, 1H), 7.25 (d, J=8.3 Hz, 1H), 6.95-6.88 (m, 1H), 6.87-6.81 (m, 1H), 5.83-5.77 (m, 2H), 4.58 (dd, J=4.4, 6.7 Hz, 1H), 4.05 (dd, J=5.0, 7.5 Hz, 1H), 3.82-3.77 (m, 1H), 3.53 (s, 3H), 3.20 (s, 3H), 1.50 (s, 9H), 0.91 (s, 9H), 0.11 (d, J=2.5 Hz, 6H).

Preparation of (7): To a solution of 6 (7.7 g, 13.71 mmol) in MeOH (10 mL) was added HCl/MeOH (4 M, 51.40 mL) at 20° C. The mixture was stirred at 20° C. for 16 hr. Upon completion, the reaction mixture was concentrated under reduced pressure to remove MeOH. The residue was purified by flash silica gel chromatography (ISCO®; 80 g SepaFlash® Silica Flash Column, Eluent of 0˜4% MeOH/DCM @ 60 mL/min) to give 7 (4.1 g, 86.11% yield) as a white solid. ESI-LCMS: 369.9 [M+Na]⁺; ¹H NMR (400 MHz, DMSO-d₆) δ=11.44 (s, 1H), 7.66 (d, J=8.3 Hz, 1H), 7.11 (q, J=4.9 Hz, 1H), 6.69 (dd, J=6.0, 15.1 Hz, 1H), 6.56-6.47 (m, 1H), 5.82 (d, J=4.0 Hz, 1H), 5.67 (dd, J=2.0, 8.0 Hz, 1H), 5.56 (br s, 1H), 4.42 (t, J=6.1 Hz, 1H), 4.13 (t, J=5.8 Hz, 1H), 3.97 (t, J=4.8 Hz, 1H), 3.39 (s, 3H), 2.48 (d, J=5.3 Hz, 3H)

Preparation of (8): To a solution of 7 (2.5 g, 7.20 mmol) in THF (25 mL) was added Pd/C (2.5 g, 10% purity) under H₂ atmosphere, and the suspension was degassed and purged with H₂ for 3 times. The mixture was stirred under H₂ (15 Psi) at 20° C. for 1 hr. Upon completion, the reaction mixture was filtered and concentrated under reduced pressure to give a residue. The residue was purified by flash silica gel chromatography (ISCO®; 25 g SepaFlash® Silica Flash Column, Eluent of 0˜5% Ethylacetate/Petroleum ether gradient @ 50 mL/min) to give 8 (2.2 g, 87.49% yield) as a white solid. ESI-LCMS: 372.1 [M+Na]⁺; ¹H NMR (400 MHz, DMSO-d₆) δ=11.40 (s, 1H), 7.62 (d, J=8.0 Hz, 1H), 6.93 (q, J=4.9 Hz, 1H), 5.76 (d, J=4.5 Hz, 1H), 5.66 (d, J=8.0 Hz, 1H), 5.26 (d, J=6.3 Hz, 1H), 3.97 (q, J=5.9 Hz, 1H), 3.91-3.79 (m, 2H), 3.36 (s, 3H), 3.14-3.00 (m, 2H), 2.56 (d, J=5.0 Hz, 3H), 2.07-1.87 (m, 2H).

Preparation of (Example 40 monomer): To a solution of 8 (2.2 g, 6.30 mmol, 1 eq) in CH₃CN (25 mL) was added P-1 (2.47 g, 8.19 mmol, 2.60 mL, 1.3 eq) at 0° C., and then 1H-imidazole-4,5-dicarbonitrile (818.07 mg, 6.93 mmol, 1.1 eq) was added in one portion at 0° C. under Ar. The mixture was stirred at 20° C. for 2 hr. Upon completion, the reaction mixture was quenched by saturated aq. NaHCO₃ (25 mL), and extracted with DCM (25 mL*2). The combined organic layers were washed with brine (25 mL*2), dried over Na₂SO₄, filtered and concentrated under reduced pressure to give a residue. The residue was purified by flash silica gel chromatography (ISCO®; 40 g SepaFlash® Silica Flash Column, Eluent of 40˜85% ethylacetate/petroleum ether gradient @ 40 mL/min) to give Example 40 monomer (2.15 g, 61.32% yield) as a white solid. ESI-LCMS: 572.2 [M+Na]⁺; ¹H NMR (400 MHz, CD₃CN) δ=9.32 (br s, 1H), 7.39 (d, J=8.1 Hz, 1H), 5.82-5.75 (m, 1H), 5.66 (dd, J=0.7, 8.1 Hz, 1H), 5.14 (qd, J=4.9, 9.4 Hz, 1H), 4.24-4.02 (m, 2H), 3.99-3.93 (m, 1H), 3.90-3.60 (m, 4H), 3.43 (d, J=17.5 Hz, 3H), 3.18-3.08 (m, 2H), 2.74-2.61 (m, 5H), 2.19-2.11 (m, 1H), 2.09-1.98 (m, 1H), 1.19 (ddd, J=2.4, 4.0, 6.6 Hz, 12H). ³¹P NMR (162 MHz, CD₃CN) δ=149.77 (s), 149.63 (br s).

### Example 41

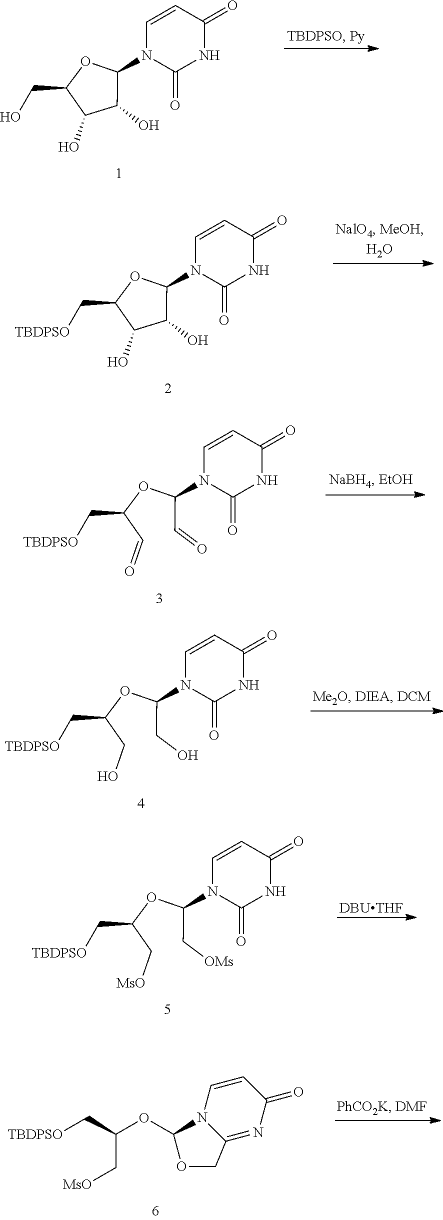

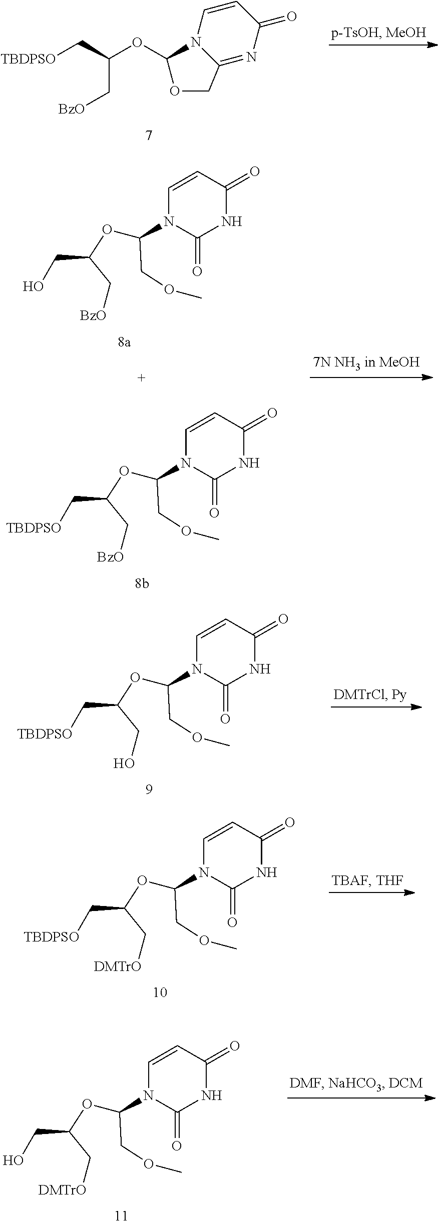

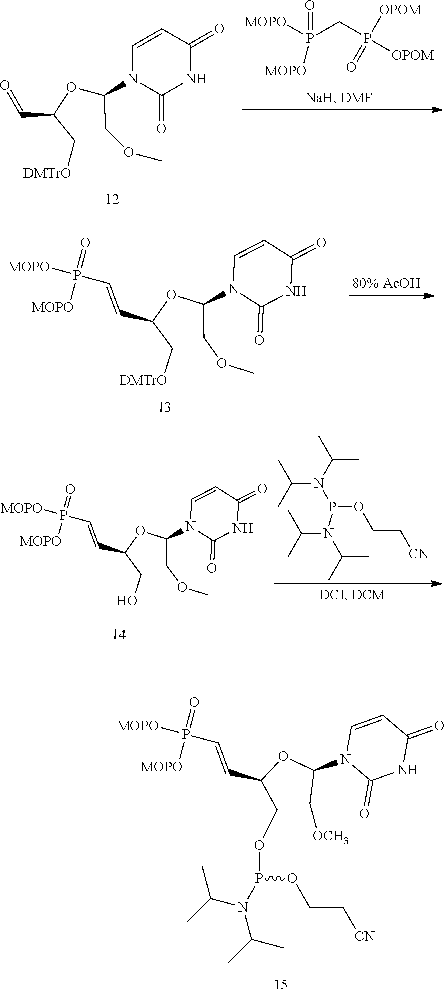

### Preparation of 2

Into a 5000-mL 3-necked round-bottom flask purged and maintained with an inert atmosphere of argon, was placed uridine (150.00 g, 614.24 mmol, 1.00 eq), pyridine (2.2 L), TBDPSCl (177.27 g, 644.95 mmol, 1.05 eq). The resulting solution was stirred overnight at room temperature. The resulting mixture was concentrated. The resulting solution was extracted with 3×1000 mL of dichloromethane and the organic layers combined. The resulting mixture was washed with 3×1 L of 0.5N HCl (aq.) and 2×500 mL of 0.5N NaHCO₃(aq.). The resulting mixture was washed with 2×1 L of H₂O. The mixture was dried over anhydrous sodium sulfate. The solids were filtered out. The filtrate was concentrated. This resulted in 262 g (crude) 2. LC-MS (m/z) 483.00 [M+H]⁺; 1 H NMR (400 MHz, DMSO-d₆) δ 11.35 (d, J=2.2 Hz, 1H), 7.70 (d, J=8.1 Hz, 1H), 7.64 (m, 4H), 7.52-7.40 (m, 6H), 5.80 (d, J=4.1 Hz, 1H), 5.50 (d, J=5.1 Hz, 1H), 5.28 (dd, J=8.0, 2.2 Hz, 1H), 5.17 (d, J=5.3 Hz, 1H), 4.15-4.05 (m, 2H), 4.00-3.85 (m, 2H), 3.85-3.73 (m, 1H), 1.03 (s, 9H).

### Preparation of 3

Into a 10 L 3-necked round-bottom flask purged and maintained with an inert atmosphere of argon, was placed a solution of 2 (260.00 g, 538.7 mmol, 1.0 eq.) in MeOH (5000 mL). This was followed by the addition of a solution of NaIO₄ (126.8 g, 592.6 mmol, 1.1 eq.) in H₂O (1600 mL) in several batches at 0° C. The resulting solution was stirred for 1 hr at room temperature. The reaction was then quenched by the addition of 3 L of Na₂S203 (sat.) at 0° C. The resulting solution was extracted with 3×1 L of dichloromethane and the organic layers combined and dried over anhydrous sodium sulfate. The solids were filtered out. The filtrate was concentrated. This resulted in 290 g (crude) of 3 as a white solid.

### Preparation of 4

Into a 5 L 3-necked round-bottom flask purged and maintained with an inert atmosphere of argon, was placed 3 (290 g, 603.4 mmol, 1.0 eq), EtOH (3 L). This was followed by the addition of NaBH₄ (22.8 g, 603.4 mmol, 1.0 eq), in portions at 0° C. The resulting solution was stirred for 1 hr at room temperature. The reaction was then quenched by the addition of 2000 mL of water/ice. The resulting solution was extracted with 3×1000 mL of dichloromethane and the organic layers combined and dried over anhydrous sodium sulfate. The solids were filtered out. The filtrate was concentrated. This resulted in 230 g (crude) of 4 as a white solid. LC-MS: m/z 485.10 [M+H]′. 1H NMR (400 MHz, DMSO-d₆) δ 11.28 (d, J=2.2 Hz, 1H), 7.63-7.37 (m, 11H), 5.84 (dd, J=6.4, 4.9 Hz, 1H), 5.44 (dd, J=8.0, 2.2 Hz, 1H), 5.11 (t, J=6.0 Hz, 1H), 4.78 (t, J=5.2 Hz, 1H), 3.65 (dd, J=11.4, 5.7 Hz, 1H), 3.60-3.52 (m, 5H), 3.18 (d, J=5.2 Hz, 1H), 0.96 (s, 9H).

### Preparation of 5

Into a 5000-mL 3-necked round-bottom flask purged and maintained with an inert atmosphere of argon, was placed a solution of 4 (120 g, 1 eq) in DCM (1200 mL). This was followed by the addition of DIEA (95.03 g, 3 eq) at 0 degrees C. To this was added methanesulfonic anhydride (129 g, 3 eq), in portions at 0° C. The resulting solution was stirred for 1 hr at room temperature. The reaction was then quenched by the addition of 1000 mL of water/ice. The resulting solution was extracted with 3×500 mL of dichloromethane and the organic layers combined and dried over anhydrous magnesium sulfate. The solids were filtered out. The filtrate was concentrated. This resulted in 160 g (crude) of 5 as a yellow solid; LC-MS (m/z) 641.05 [M+H]⁺.

### Preparation of 6

Into a 1 L round-bottom flask, was placed a solution of 5 (160.00 g, 1.00 equiv) in THF (1600 mL), DBU (108 g, 2.8 equiv). The resulting solution was stirred for 1 hr at 30° C. The reaction was then quenched by the addition of 3000 mL of water/ice. The resulting solution was extracted with 3×500 mL of dichloromethane and the organic layers combined and dried over anhydrous sodium sulfate. The solids were filtered out. The filtrate was concentrated. This resulted in 150 g (crude) of 6 as brown oil; LC-MS: (ES, m/z): 567.25 [M+H]⁺

¹ HNMR (400 MHz, DMSO-d₆) δ 7.83 (d, J=7.4 Hz, 1H), 7.67-7.55 (m, 4H), 7.55-7.35 (m, 6H), 6.05 (dd, J=5.9, 1.7 Hz, 1H), 5.72 (d, J=7.4 Hz, 1H), 4.81 (dd, J=10.4, 5.8 Hz, 1H), 4.58-4.46 (m, 2H), 4.42 (p, J=5.2, 4.6 Hz, 1H), 4.33 (dd, J=10.6, 5.9 Hz, 1H), 3.79-3.70 (m, 2H), 3.23 (s, 3H), 0.98 (s, 9H).

### Preparation of 7

Into a 3000-mL round-bottom flask purged and maintained with an inert atmosphere of argon, was placed 6 (150.00 g, 201.950 mmol, 1. eq), DMF (1300.00 mL), potassium benzoate (44.00 g, 1.0 eq). The resulting solution was stirred for 1.5 hr at 80° C. The reaction was then quenched by the addition of 500 ml of water/ice. The resulting solution was extracted with 3×500 mL of dichloromethane The resulting mixture was washed with 3×1000 ml of H₂O. The resulting mixture was concentrated. The residue was applied onto a silica gel column with EA/PE (99:1). The collected fractions were combined and concentrated. This resulted in 40 g of 7 as yellow oil. LC-MS: m/z 571.20 [M+H]⁺; ¹HNMR: (400 MHz, DMSO-d₆) δ 7.97-7.91 (m, 2H), 7.89 (d, J=7.4 Hz, 1H), 7.74-7.51 (m, 7H), 7.51-7.31 (m, 6H), 6.16 (m, 1H), 5.76 (d, J=7.4 Hz, 1H), 4.78 (m, 1H), 4.61 (m, 1H), 4.55-4.46 (m, 2H), 4.38 (m, 1H), 3.82 (d, J=5.0 Hz, 2H), 0.97 (s, 9H)

### Preparation of 8b

Into a 2-L round-bottom flask, was placed 7 (30.00 g, 1 eq), MeOH (1.20 L), p-toluenesulfonic acid (4.50 g, 0.5 eq). The resulting solution was stirred for 2 hr at 70° C. The reaction was then quenched by the addition of 3 L of NaHCO₃ (sat.). The pH value of the solution was adjusted to 7 with NaHCO₃ (sat.). The resulting solution was extracted with 3×1 L of ethyl acetate and the organic layers combined and dried over anhydrous sodium sulfate. The solids were filtered out. The filtrate was concentrated under vacuum. The crude product was purified by Flash-Prep-HPLC with the following conditions (IntelFlash-1): Column, silica gel; mobile phase, PE/EA=50/50 increasing to PE/EA=25/75 within 30; Detector, 254. This resulted in 11.5 g (3.1% yield in seven steps) 8b as a white solid. LC-MS: m/z 625.15 [M+Na]⁺; ¹HNMR: (400 MHz, DMSO-d₆) δ 11.37 (d, J=2.3 Hz, 1H), 7.99-7.93 (m, 2H), 7.74-7.65 (m, 1H), 7.63-7.50 (m, 7H), 7.50-7.33 (m, 6H), 6.08 (t, J=6.0 Hz, 1H), 5.49 (m, 1H), 4.60 (m, 1H), 4.43 (m, 1H), 4.03-3.96 (m, 1H), 3.70 (d, J=5.3 Hz, 2H), 3.62-3.49 (m, 2H), 3.21 (s, 3H), 0.97 (s, 9H).

### Preparation of 9

Into a 2-L round-bottom flask, was placed 8b

(11.50 g). To the above 7M NH₃ (g) in MeOH (690.00 mL) was introduced in at 30° C. The resulting solution was stirred overnight at 30 degrees C. The resulting mixture was concentrated under vacuum. The crude product was purified by Flash with the following conditions (IntelFlash-1): Column, silica gel; mobile phase, PE/EA-60/40 increasing to PE/EA=1/99 within 60; Detector, 254. This resulted in 8.1 g (97% yield) of 9 as a white solid. LC-MS−: m/z 499.35 [M+H]⁺; ¹HNMR−: (300 MHz, DMSO-d₆) δ 11.31 (s, 1H), 7.64-7.50 (m, 5H), 7.48-7.35 (m, 6H), 6.02 (t, J=5.8 Hz, 1H), 5.45 (d, J=8.0 Hz, 1H), 4.80 (t, J=5.1 Hz, 1H), 3.58 (m, 7H), 3.27 (s, 3H), 0.96 (s, 9H).

### Preparation of 10

Into a 250-mL round-bottom flask, was placed 9 (8.10 g, 1 equiv), pyridine (80.0 mL), DMTr-Cl (7.10 g, 1.3 eq). The flask was evacuated and flushed three times with Argon. The resulting solution was stirred for 2 hr at room temperature. The reaction was then quenched by the addition of 500 mL of NaHCO₃ (sat.). The resulting solution was extracted with 2×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 11.5 g (88% yield) of 10 as a white solid; LC-MS: m/z 823.40 [M+Na]⁺; 1HNMR: (300 MHz, DMSO-d₆) δ 11.37 (s, 1H), 7.55-7.18 (m, 20H), 6.92-6.83 (m, 4H), 6.14 (t, J=5.9 Hz, 1H), 5.48 (d, J=8.0 Hz, 1H), 3.74 (m, 7H), 3.57 (m, 4H), 3.25 (m, 5H), 0.84 (s, 9H).

### Preparation of 11

Into a 1000-mL round-bottom flask, was placed 10 (11.5 g, 1.00 eq), THF (280.00 mL), TBAF (14.00 mL, 1.00 eq). The resulting solution was stirred for 3 hr at room temperature. The reaction was then quenched by the addition of 1 L of water. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.8 g (98% yield) of 11 as a white solid. LC-MS: m/z 561.20 [M−H]⁻; ¹HNMR: (300 MHz, DMSO-d₆) δ 11.32 (s, 1H), 7.66 (d, J=8.1 Hz, 1H), 7.52-7.39 (m, 2H), 7.39-7.20 (m, 7H), 6.96-6.83 (m, 4H), 6.17 (t, J=5.9 Hz, 1H), 5.63 (d, J=8.0 Hz, 1H), 4.63 (t, J=5.6 Hz, 1H), 3.90-3.46 (m, 9H), 3.26 (s, 5H), 3.19-2.98 (m, 2H).

### Preparation of 12

Into a 3-L round-bottom flask, was placed 11 (7.80 g, 1.00 eq), DCM (300.00 mL), NaHCO₃ (3.50 g, 3 eq). This was followed by the addition of Dess-Martin (7.06 g, 1.2 equiv) with stirring at 0° C., and the resulting solution was stirred for 20 min at 0° C. The resulting solution was stirred for 5 hr at room temperature. The reaction mixture was cooled to 0 degree C. with a water/ice bath. The reaction was then quenched by the addition of 500 mL of NaHCO₃: Na₂S203=1:1. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 5.8 g (75% yield) of 12 as a white solid. LC-MS: m/z 558.80 [M−H]⁻; ¹HNMR−: (300 MHz, DMSO-d₆) δ 11.35-11.22 (m, 1H), 9.43 (s, 1H), 7.75 (d, J=8.1 Hz, 1H), 7.49-7.19 (m, 8H), 6.90 (m, 5H), 6.00 (t, J=5.9 Hz, 1H), 5.66 (m, 1H), 4.40 (m, 1H), 3.75 (s, 7H), 3.70-3.56 (m, 3H), 3.29 (d, J=3.7 Hz, 3H).

### Preparation of 13

Into a 250-mL 3-round-bottom flask, was placed THF (150.00 mL), NaH (1.07 g, 60% w, 3.00 equiv). The flask was evacuated and flushed three times with Argon, and the reaction mixture was cooled to −78° C. This was followed by the addition of [[(bis[[(2,2-dimethylpropanoyl)oxy]methoxy]phosphoryl)methyl([(2,2-dimethylpropanoyl)oxy]methoxy)phosphoryl]oxy]methyl 2,2-dimethylpropanoate (14.60 g, 2.6 eq, in 60 m L THF) dropwise with stirring at −78° C. in 10 min, and the resulting solution was stirred for 30 min at −78° C. This was followed by the addition of 12 (5.00 g, 1.00 eq, in 50 mL THF) dropwise with stirring at −78° C. in 10 min. The resulting solution was stirred for 4 hr at room temperature. The reaction was then quenched by the addition of 400 mL of NH₄Cl (sat.). The resulting solution was extracted with 3×400 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.2 g (crude) of 13 as a solid. LC-MS: m/z: 865.10 [M−H].⁻

### Preparation of 14

Into a 500-mL round-bottom flask, was placed 13

(6.00 g), H₂O (30.00 mL), AcOH (120.00 mL). The resulting solution was stirred for 1 hr at 50 degrees C. The reaction mixture was cooled to 0 degree C. with a water/ice bath. The reaction was then quenched by the addition of 2 L of NaHCO₃ (sat.). The pH value of the solution was adjusted to 7 with NaHCO₃ (sat.). The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 2.6 g (44% yield in two steps) of 14 as yellow oil. LC-MS: m/z 587.25 [M+Na]⁺; ¹HNMR: (300 MHz, DMSO-d₆) δ 11.31 (s, 1H), 7.73 (d, J=8.1 Hz, 1H), 6.63 (ddd, J=24.2, 17.2, 4.2 Hz, 1H), 6.14-5.96 (m, 2H), 5.65-5.48 (m, 5H), 5.09 (t, J=5.6 Hz, 1H), 4.17 (s, 1H), 3.65 (d, J=6.1 Hz, 2H), 3.52 (m, 2H), 3.27 (s, 3H), 1.15 (d, J=3.7 Hz, 18H); ³¹PNMR−: (162 MHz, DMSO-d₆) δ 17.96.

### Preparation of 15

Into a 250-mL 3-necked round-bottom flask, was placed DCM (60.00 mL), DCI (351.00 mg, 1.2 eq), 3-[[bis(diisopropylamino)phosphanyl]oxy]propanenitrile (971.00 mg, 1.3 eq), 4A MS. The flask was evacuated and flushed three times with Argon, and the reaction mixture was cooled to 0° C. This was followed by the addition of 14 (1.40 g, 1.00 eq, in 30 mL DCM) dropwise with stirring at 0° C. in 30 second. The resulting solution was stirred for 1 hr at room temperature. The reaction was then quenched by the addition of 50 mL of water. The resulting solution was extracted with 3×50 mL of ethyl acetate and the organic layers combined. The resulting mixture was washed with 3×50 ml of NaCl (sat.). The mixture was dried over anhydrous magnesium sulfate. The solids were filtered out. The filtrate was concentrated under vacuum. The crude product was purified by Prep-Archiral-SFC with the following conditions: Column: Ultimate Diol, 2*25 cm, 5 [symbol] Ìm; Mobile Phase A: CO₂, Mobile Phase B: ACN (0.2% TEA); Flow rate: 50 mL/min; Gradient: isocratic 30% B; Column Temperature (20° C.): 35; Back Pressure (bar): 100; Wave Length: 254 nm; RT1 (min): 2.58; Sample Solvent: MeOH-HPLC; Injection Volume: 1 mL; Number Of Runs: 4. This resulted in 1.31 g (65% yield) 15 as yellow oil. LC-MS: m/z 763.40 [M−H]⁻; ¹HNMR−: (300 MHz, Acetonitrile-d₃) δ 9.05 (s, 1H), 7.51 (d, J=8.1 Hz, 1H), 6.64 (dddd, J=23.8, 17.1, 4.8, 1.9 Hz, 1H), 6.23-5.92 (m, 2H), 5.70-5.51 (m, 5H), 4.38 (d, J=4.9 Hz, 1H), 3.96-3.56 (m, 8H), 3.35 (s, 3H), 2.70 (m, 2H), 1.33-1.14 (m, 30H); 31 PNMR−: (Acetonitrile-d₃) δ 148.75, 148.53, 16.68.

### Example 42

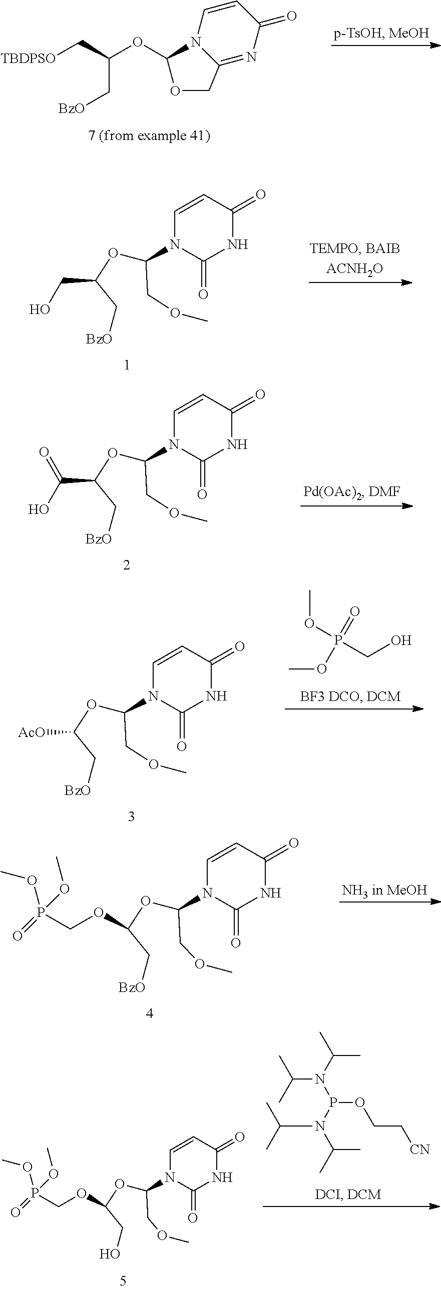

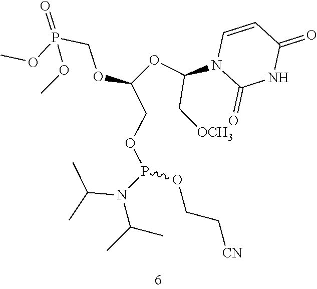 [SMILES: COC[C@@H](O[C@@H](COP(OCCC#N)N(C(C)C)C(C)C)OCP(=O)(OC)OC)n1ccc(=O)[nH]c1=O]

### Preparation of 1

A solution of 7 from Example 41 (23 g, 40.300 mmol, 1.00 equiv) and p-TsOH (9.02 g, 52.390 mmol, 1.3 equiv) in MeOH (1000 mL) was stirred for overnight at 40° C. under argon atmosphere. The reaction was quenched with sat. sodium bicarbonate (aq.) at 0 degrees C. The resulting mixture was extracted with EtOAc (2×500 mL). The combined organic layers were washed with water (2×500 mL), dried over anhydrous MgSO₄. After filtration, the filtrate was concentrated under reduced pressure. The residue was purified by reverse flash chromatography with the following conditions: column, C18 silica gel; mobile phase, ACN in water, 10% to 90% gradient in 30 min; detector, UV 254 nm. This resulted in 1 (5.3 g, 36. %) as a colorless oil; LC-MS: (ES, m/z): 365 [M+H]⁺; ¹H-NMR: (300 MHz, DMSO-d₆) δ 11.20 (s, 1H), 8.09-7.78 (m, 2H), 7.63-7.50 (m, 2H), 7.51-7.35 (m, 2H), 5.95 (t, J=5.9 Hz, 1H), 5.51 (d, J=8.1 Hz, 1H), 4.73 (t, J=5.7 Hz, 1H), 4.41 (dd, J=11.9, 3.3 Hz, 1H), 4.17 (dd, J=11.9, 6.3 Hz, 1H), 3.69 (dq, J=10.1, 6.8, 6.3 Hz, 1H), 3.48-3.40 (m, 2H), 3.39-3.29 (m, 2H), 3.07 (s, 3H).

### Preparation of 2

Into a 250-mL 3-necked round-bottom flask, was placed 1 (7.00 g, 19.212 mmol, 1.00 equiv), ACN (60.00 mL), H₂O (60.00 mL), TEMPO (0.72 g, 4.611 mmol, 0.24 equiv), BAIB (13.61 g, 42.267 mmol, 2.20 equiv). The resulting solution was stirred for 1 overnight at 30° C. The reaction was then quenched by the addition of 200 ml of water/ice. The resulting solution was extracted with 2×200 mL of ethyl acetate, The resulting mixture was washed with 2×200 ml of water. The mixture was dried over anhydrous sodium sulfate and concentrated. The crude product was purified by Flash-Prep-HPLC with the following conditions (IntelFlash-1): Column, C18 silica gel; mobile phase, ACN/H₂O=5/95 increasing to ACN/H₂O=95/5 within 30 min; Detector, UV 254 nm; product was obtained. This resulted in 5 g (68.8%) of 2 as a solid. LC-MS: (ES, m/z): 379 [M+H]⁺; ¹H NMR (300 MHz, DMSO-d₆) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H).

### Preparation of 3

Into a 250-mL round-bottom flask, was placed 2 (4.5 g, 11.894 mmol, 1.00 equiv), DMF (90.00 mL), Pb(OAc)₄ (15.82 g, 35.679 mmol, 3.00 equiv). The resulting solution was stirred overnight at 30° C. The reaction was then quenched by the addition of 200 mL of water/ice. The resulting solution was extracted with 2×200 mL of ethyl acetate The resulting mixture was washed with 2×200 ml of water. The mixture was dried over anhydrous sodium sulfate and concentrated. The crude product was purified by Flash with the following conditions (IntelFlash-1): Column, C18 silica gel; mobile phase, ACN/H₂O-5/95 increasing to ACN/H₂O=95/5 within 30 min; Detector, UV 254 nm; product was obtained. This resulted in 4 g 3 as oil; LC-MS: (ES, m/z): 415 [M+Na]⁺; ¹H NMR (300 MHz, DMSO-d₆) δ 11.39 (s, 1H), 7.93 (dd, J=24.2, 7.6 Hz, 2H), 7.75-7.46 (m, 4H), 6.35-6.03 (m, 2H), 5.71-5.47 (m, 1H), 4.60-4.14 (m, 2H), 3.88-3.54 (m, 2H), 3.26 (d, J=6.7 Hz, 3H), 2.03 (d, J=49.7 Hz, 3H).

### Preparation of 4

Into a 250-mL 3-necked round-bottom flask purged and maintained with an inert atmosphere of argon, was placed 3 (4.00 g, 10.195 mmol, 1.00 eq), DCM (80.00 mL), dimethyl hydroxymethylphosphonate (22.85 g, 163.114 mmol, 16.00 eq), BF₃·Et₂O (28.94 g, 203.91 mmol, 20 eq). The resulting solution was stirred overnight at room temperature. The reaction was then quenched by the addition of 500 mL of water/ice. The resulting solution was extracted with 2×500 mL of ethyl acetate The resulting mixture was washed with 2×500 ml of water. The mixture was dried over anhydrous sodium sulfate and concentrated. The residue was applied onto a silica gel column with dichloromethane/methanol (20/1). This resulted in 2 g (41.5%) of 4 as a solid.

LC-MS: (ES, m/z): 490 [M+H₂O]⁺; ¹H-NMR (300 MHz, DMSO-d₆) δ 11.39 (d, J=5.4 Hz, 1H), 7.96 (dt, J=11.5, 9.3 Hz, 2H), 7.81-7.40 (m, 4H), 6.29-5.98 (m, 1H), 5.56 (dd, J=12.2, 8.1 Hz, 1H), 5.28-4.99 (m, 1H), 4.29 (dp, J=25.1, 5.9 Hz, 2H), 4.16-3.84 (m, 2H), 3.75-3.53 (m, 7H), 3.28 (d, J=12.5 Hz, 2H).

### Preparation of 5

Into a 100-mL round-bottom flask, was placed 4 (2.00 g, 4.234 mmol, 1.00 equiv), 7M NH₃ (g) in THF (20.00 mL) was added. The resulting solution was stirred overnight at 25° C. The resulting mixture was concentrated under vacuum. The crude product was purified by prep-sfc Column: Lux 5 um i-Cellulose-5, 3*25 cm, 5 um; Mobile Phase A: CO₂, Mobile Phase B: MeOH (0.1% 2M NH₃-MEOH); Flow rate: 70 mL/min; Gradient: isocratic 50% B; Column Temperature (25° C.): 35; Back Pressure (bar): 100; Wave Length: 220 nm; RT1 (min): 3.75; RT2 (min): 4.92; Sample Solvent: MeOH:DCM=1:1; Injection Volume: 1 mL; Number Of Runs: 15, This resulted in 330 mg (21.2%) of 5 as a solid. ¹H-NMR−: (300 MHz, DMSO-d₆) δ 11.14 (s, 1H), 7.63 (d, J=8.1 Hz, 1H), 6.06 (t, J=5.9 Hz, 1H), 5.64 (d, J=8.0 Hz, 1H), 4.89 (s, 1H), 4.63 (t, J=5.3 Hz, 1H), 3.98 (d, J=9.8 Hz, 2H), 3.70 (dd, J=10.7, 1.2 Hz, 8H), 3.63 (dd, J=6.0, 3.2 Hz, 1H), 3.29 (s, 3H).

### Preparation of 6

To a stirred solution of 3-{[bis(diisopropylamino)phosphanyl]oxy}propanenitrile (324.10 mg, 1.075 mmol, 1.2 equiv) and 1H-imidazole-4,5-dicarbonitrile (126.99 mg, 1.075 mmol, 1.2 equiv) in DCM (10 mL) was added 5 (330 mg, 0.9 mmol, 1.00 eq) dropwise at 25° C. under argon atmosphere. The resulting mixture was stirred for 30 min at 25 degrees C. The reaction was quenched with water/ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 μm; Mobile Phase A: CO₂, Mobile Phase B: ACN; Flow rate: 50 mL/min; Gradient: isocratic 30% B; Column Temperature (25° C.): 35; Back Pressure (bar): 100; Wave Length: 254 nm; RT1 (min): 3.95; Sample Solvent: ACN; Injection Volume: 1 mL; Number Of Runs: 10, This resulted in 6 (349 mg, 68.4%) as a light yellow oil. LC-MS: (ES, m/z): 567.25 [M+H]⁺; ¹H-NMR: (300 MHz, DMSO-d₆) δ 11.38 (s, 1H), 7.64 (dd, J=8.0, 1.3 Hz, 1H), 6.09 (dt, J=5.8, 3.4 Hz, 1H), 5.65 (dd, J=8.0, 3.2 Hz, 1H), 4.83 (q, J=5.5 Hz, 1H), 4.03 (dt, J=9.7, 2.2 Hz, 2H), 3.83-3.40 (m, 14H), 3.30 (s, 3H), 2.77 (t, J=5.9 Hz, 2H), 1.12 (ddd, J=9.2, 6.7, 1.7 Hz, 12H); ³¹P NMR (DMSO-d₆) δ 148.0, 147.6, 23.1

### Example 43

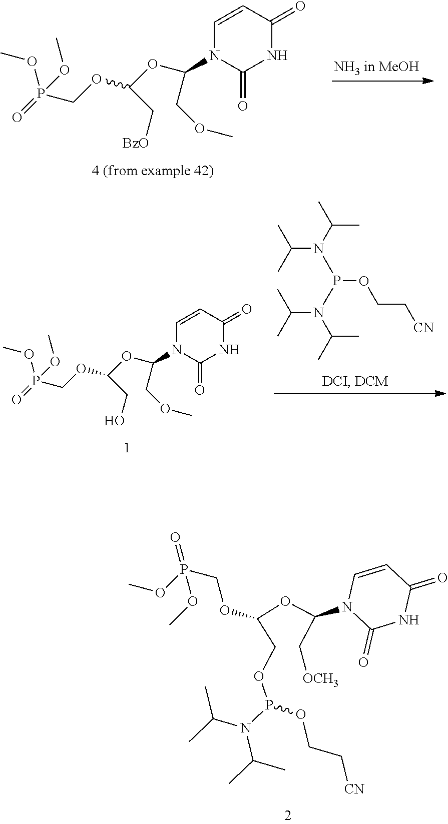

### Preparation of 1

Into a 100-mL round-bottom flask, was placed 2 4 from Example 42 (2.00 g, 4.234 mmol, 1.00 equiv), 7M NH₃(g) in THF (20.00 mL) was added. The resulting solution was stirred overnight at 25° C. The resulting mixture was concentrated under vacuum. The crude product was purified by prep-sfc Column: Lux 5 um i-Cellulose-5, 3*25 cm, 5 μm; Mobile Phase A: CO₂, Mobile Phase B: MeOH (0.1% 2M NH₃-MeOH); Flow rate: 70 mL/min; Gradient: isocratic 50% B; Column Temperature (C): 35; Back Pressure (bar): 100; Wave Length: 220 nm; RT1 (min): 3.75; RT2 (min): 4.92; Sample Solvent: MeOH:DCM=1:1; Injection Volume: 1 mL; Number Of Runs: 15, This resulted in 320 mg (22.8%) of 1 as a solid. 1H-NMR− −14-3-40: (300 MHz, DMSO-d₆) δ 11.11 (s, 1H), 7.70 (d, J=8.0 Hz, 1H), 6.03 (t, J=6.1 Hz, 1H), 5.64 (d, J=8.0 Hz, 1H), 4.97 (s, 1H), 4.76 (t, J=5.3 Hz, 1H), 4.07-3.85 (m, 1H), 3.79 (dd, J=13.9, 9.3 Hz, 1H), 3.73-3.55 (m, 9H), 3.41 (d, J=5.0 Hz, 2H), 3.28 (s, 3H).

### Preparation of 2

To a stirred solution/mixture of 3-{[bis(diisopropylamino)phosphanyl]oxy}propanenitrile (517.58 mg, 1.717 mmol, 1.2 equiv) and 1H-imidazole-4,5-dicarbonitrile (202.79 mg, 1.717 mmol, 1.2 equiv) in DCM was added 1 (527 mg, 1.431 mmol, 1.00 eq.) dropwise at 25° C. under argon atmosphere. The resulting mixture was stirred for 30 min at 25° C. The reaction was quenched with Water/Ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 μm; Mobile Phase A: CO₂, Mobile Phase B: ACN (0.1% DEA)-HPLC-merk; Flow rate: 50 mL/min; Gradient: isocratic 30% B; Column Temperature (C): 35; Back Pressure (bar): 100; Wave Length: 254 nm; RT1 (min): 4.57; Sample Solvent: ACN; Injection Volume: 1 mL; Number Of Runs: 10 to afford 2 (264.8 mg, 31.7%) as a light yellow oil. LC-MS: (ES, m/z): 567.25 [M−H]⁻; ¹H NMR (300 MHz, DMSO-d₆) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H); ³¹P NMR (DMSO-d₆) δ 148.01, 147.67, 22.8.

### Example 44

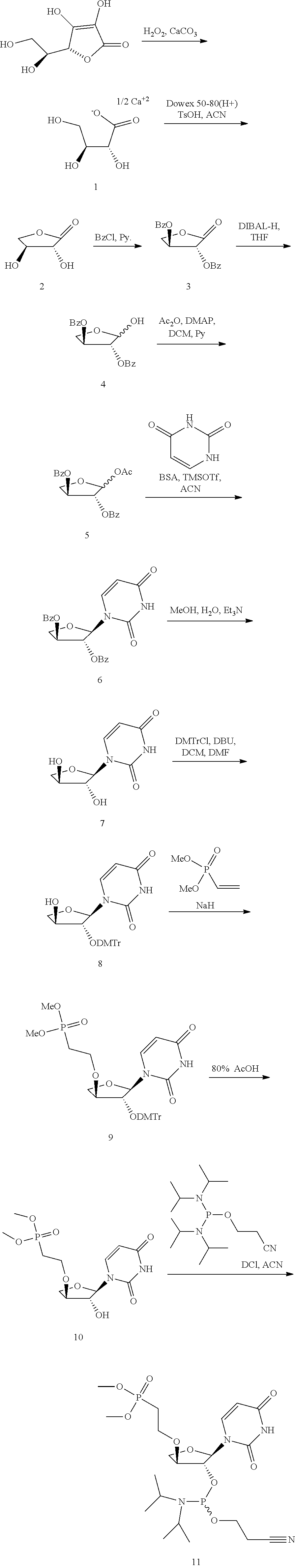

### Preparation of 1

To a stirred mixture of ascorbic acid (100.00 g, 567.78 mmol, 1.00 equiv) and CaCO₃ (113.0 g, 1129.02 mmol, 2 equiv) in H₂O (1.00 L) was added H₂O₂ (30%) (236.0 g, 6938.3 mmol, 12.22 equiv) dropwise at 0° C. The resulting mixture was stirred overnight at room temperature. The mixture was treat with charcoal and heat to 70 degrees until the no more peroxide was detected. The resulting mixture was filtered, the filter cake was washed with warm water (3×300 mL). The filtrate was concentrated under reduced pressure. The solid was diluted with MeOH (200 mL) and the mixture was stirred for 5 h. The resulting mixture was filtered, the filter cake was washed with MeOH (3×80 mL). The filtrate was concentrated under reduced pressure to afford L-threonate (86 g, 96.6%) as a white crude solid. ¹H-NMR−: (300 MHz, Deuterium Oxide) δ 4.02 (dd, J=4.6, 2.4 Hz, 1H), 3.91 (ddt, J=7.6, 5.3, 2.2 Hz, 1H), 3.78-3.44 (m, 2H).

### Preparation of 2

Into a 5 L round-bottom flask were added L-threonate (70.00 g, 518.150 mmol, 1.00 equiv) and H₂O (2 L) at room temperature. The residue was acidified to pH=1 with Dowex 50wX8, H(+)-Form). The resulting mixture was stirred for 1 h at 70° C. The resulting mixture was filtered, the filter cake was washed with water (2×1 L). The filtrate was concentrated under reduced pressure. The solid was co-evaporated with (2×2 L). Then the solid was diluted with ACN (700.00 mL), and the TsOH (5.35 g, 31.089 mmol, 0.06 equiv) was added. The resulting mixture was stirred for 1 h at 80 degrees C. under air atmosphere. The resulting mixture was filtered, the filter cake was washed with ACN (2×500 mL). The filtrate was concentrated under reduced pressure to 2 (70 g, crude) as a yellow oil.

### Preparation of 3

To a stirred solution of (2 (70.0 g crude, 593.2 mmol, 1.00 eq.) in pyridine (280.00 mL) was added benzoyl chloride (207.62 g, 1.483 mol, 2.5 equiv) dropwise at 0° C. under argon atmosphere. The resulting mixture was stirred for 1 h at room temperature under argon atmosphere. The reaction was quenched by the addition of sat. NaHCO₃(aq.) (500 mL) at 0 degrees C. The resulting mixture was extracted with CH₂Cl₂ (3×500 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc to afford (3 (80 g, 41.4%) as an off-white solid. LC-MS: (ES, m/z): 327 [M+H]⁺; ¹H-NMR: (300 MHz, CDCl₃) δ 8.18-8.04 (m, 4H), 7.68-7.61 (m, 2H), 7.50 (tt, J=7.1, 1.4 Hz, 4H), 5.96-5.57 (m, 2H), 5.11-5.00 (m, 1H), 4.45-4.35 (m, 1H).

### Preparation of 4

To a stirred solution of 3 (125 g, 383.078 mmol, 1.00 eq) in THF (1.50 L) was added DIBAL-H (1M) (600 mL, 2 eq) dropwise at −78° C. under argon atmosphere. The resulting mixture was stirred for 1 h at −78 degrees C. under argon atmosphere. Desired product was detected by LCMS. The reaction was quenched with MeOH at 0° C. The resulting mixture was diluted with EtOAc (600 mL). Then the resulting mixture was filtered, the filter cake was washed with EtOAc (3×800 mL). The filtrate was concentrated under reduced pressure. This resulted in 4 (73 g, crude) as a colorless solid. LC-MS: (ES, m/z): 392 [M+Na+ACN]+; 1H-NMR−: (400 MHz, Chloroform-d) δ 8.22-7.99 (m, 8H), 7.62 (dtd, J=7.4, 4.4, 2.2 Hz, 4H), 7.48 (td, J=7.8, 2.4 Hz, 8H), 5.87 (d, J=4.3 Hz, 1H), 5.77 (dt, J=6.6, 3.6 Hz, 1H), 5.56 (d, J=4.9 Hz, 2H), 5.50 (t, J=4.3 Hz, 1H), 4.73 (s, 1H), 4.63 (ddd, J=10.4, 7.9, 6.1 Hz, 2H), 4.28 (dd, J=10.3, 3.8 Hz, 1H), 3.99 (dd, J=10.6, 3.2 Hz, 1H).

### Preparation of 5

To a stirred solution of (4 (73.00 g, 222.344 mmol, 1.00 equiv) and DMAP (271.63 mg, 2.223 mmol, 0.01 equiv) and pyridine (365.00 mL) in DCM (365.00 mL) were added Ac₂O (24.97 g, 244.6 mmol, 1.1 equiv) dropwise at 0 degrees C. under argon atmosphere. The resulting mixture was stirred for 1 h at room temperature under argon atmosphere. The reaction was quenched with sat. NaHCO₃(aq.) at 0 degrees C. The resulting mixture was extracted with CH₂Cl₂ (3×500 mL). The combined organic layers were washed with sat. CuSO₄ (3×200 mL), dried over anhydrous Na₂SO₄. After filtration, the filtrate was concentrated under reduced pressure. The residue was purified by silica gel column chromatography, eluted with PE/EtOAc to afford 5 (60 g, 73%) as a colorless oil. LC-MS: (ES, m/z): 434 [M+Na+ACN]⁺; 1H-NMR: (400 MHz, Chloroform-d) δ 8.17-8.02 (m, 8H), 7.63 (tddd, J=7.9, 6.6, 3.2, 1.6 Hz, 4H), 7.57-7.44 (m, 8H), 6.66 (d, J=4.5 Hz, 1H), 6.40 (s, 1H), 5.83-5.53 (m, 4H), 4.67 (ddd, J=23.4, 10.5, 6.2 Hz, 2H), 4.24 (dd, J=10.5, 3.8 Hz, 1H), 4.19-4.01 (m, 1H), 2.18 (s, 3H), 2.06 (d, J=3.2 Hz, 3H).

### Preparation of 6

To a stirred mixture of 5 (50.00 g, 135.005 mmol, 1.00 eq) and uracil (15.13 g, 135.005 mmol, 1 eq) in can (500.00 mL) was added BSA (54.81 g, 270.010 mmol, 2 eq) in portions at room temperature under air atmosphere. The resulting mixture was stirred for 1 h at 60° C. under argon atmosphere. After that, the TMSOTf (90.02 g, 405.0 mmol, 3 eq) was added dropwise at 0° C. The resulting mixture was stirred for 2 h at 60° C. under argon atmosphere. The mixture was neutralized to pH=7 with saturated NaHCO₃(aq.) at 0° C. The resulting mixture was extracted with CH₂Cl₂ (3×400 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EtOAc (1:1) to afford 6 (43 g, 75.4%) as a white solid. LC-MS: (ES, m/z): [M+H]⁺; 423 464 [M+H+ACN]+; 1H-NMR−: (300 MHz, Chloroform-d) δ 9.08-8.89 (m, 1H), 8.17-7.94 (m, 4H), 7.70-7.43 (m, 7H), 6.19 (d, J=1.9 Hz, 1H), 5.84-5.71 (m, 2H), 5.62 (td, J=3.3, 2.8, 1.4 Hz, 1H), 4.59-4.44 (m, 2H), 4.14 (q, J=7.2 Hz, 1H).

### Preparation of 7

A solution of 6 (52.00 g, 123.108 mmol, 1 eq) was dissolved in 642 ml of MeOH/H₂O/TEA (5:1:1) at room temperature and heat to reflux until no more starting material was detected (2˜3 h). The resulting mixture was concentrated under reduced pressure. The residue was dissolved in EtOAc (600 mL) and the organic layer was extracted with water (5×800 mL). The aqueous layer was concentrated under vacuum to afford 7 (21 g, crude) as a off-white solid. The crude product was used in the next step directly without further purification. LC-MS−: (ES, m/z): 213 [M−H]⁻; 1 H-NMR: (300 MHz, DMSO-d₆) δ 11.26 (s, 1H), 7.68 (d, J=8.1 Hz, 1H), 5.75 (s, 1H), 5.65 (d, J=1.2 Hz, 1H), 5.59 (d, J=8.1 Hz, 1H), 5.39 (s, 1H), 4.10-3.97 (m, 4H).

### Preparation of 8

To a stirred mixture of 7 (16.00 g, 74.705 mmol, 1.00 equiv) and DBU (22.75 g, 149.409 mmol, 2 equiv) in DCM (80.00 mL) and DMF (200.00 mL) was added DMTr-Cl (7.88 g, 25.680 mmol, 1.1 equiv) dropwise at room temperature under argon atmosphere. The resulting mixture was stirred for 2 h at room temperature under argon atmosphere. The reaction was quenched by the addition of sat. NaHCO₃(aq.) (100 mL) at 0 degrees C. The resulting mixture was extracted with EtOAc (3×60 mL). The combined organic layers were washed with brine (2×50 mL), dried over anhydrous Na₂SO₄. After filtration, the filtrate was concentrated under reduced pressure. The residue was purified by silica gel column chromatography, eluted with PE (0.5% TEA)/EtOAc (2:3) to afford 8 (25 g, 64.8%) as a off-white solid; LC-MS: (ES, m/z): 515 [M−H]⁻; ¹H-NMR: (400 MHz, DMSO-d₆) δ 11.33 (s, 1H), 7.57 (d, J=8.1 Hz, 1H), 7.45-7.13 (m, 9H), 6.86 (t, J=8.5 Hz, 4H), 5.94 (d, J=1.7 Hz, 1H), 5.58 (d, J=8.1 Hz, 1H), 5.15 (d, J=2.6 Hz, 1H), 3.97-3.79 (m, 3H), 3.73 (d, J=2.3 Hz, 6H), 3.33 (d, J=2.5 Hz, 1H).

### Preparation of 9

To a stirred solution of 8 (6.00 g, 11.616 mmol, 1.00 eq) in THF (240.00 mL) was added NaH (60%) (1.40 g, 35.003 mmol, 3 eq) dropwise at 0° C. under argon atmosphere. The resulting mixture was stirred for 30 min at 0 degrees C. under argon atmosphere. Then the dimethyl ethenylphosphonate (15.81 g, 116.2 mmol, 10.00 eq) was added and the resulting mixture was stirred overnight at room temperature under argon atmosphere. The reaction was quenched with sat. NH₄Cl (aq.) at room temperature. The resulting mixture was extracted with EtOAc (3×100 mL). The combined organic layers were washed with brine (3×80 mL), dried over anhydrous Na₂SO₄. After filtration, the filtrate was concentrated under reduced pressure. The residue was purified by reverse flash chromatography with the following conditions: column, C18 mobile phase, ACN in water, 5% to 95% gradient in 30 min; detector, UV 254 nm to afford 9 (3.65 g, 48.15%) as a white solid.

LC-MS: (ES, m/z): 675 [M+Na]+; 1H-NMR−: (300 MHz, DMSO-d₆) δ 11.39 (s, 1H), 7.44-7.36 (m, 3H), 7.34-7.21 (m, 7H), 6.93-6.83 (m, 4H), 6.08 (d, J=2.0 Hz, 1H), 5.55 (d, J=8.1 Hz, 1H), 4.08 (d, J=11.0 Hz, 1H), 3.92 (d, J=2.0 Hz, 1H), 3.82-3.71 (m, 7H), 3.57 (dd, J=10.9, 3.6 Hz, 6H), 3.30-3.23 (m, 1H), 3.06-2.86 (m, 2H), 1.96 (dt, J=18.1, 7.1 Hz, 2H).

### Preparation of 10

A solution of 9 (2.80 g, 4.3 mmol, 1.00 equiv) in AcOH (12.00 mL) and H₂O (3.00 mL) was stirred for overnight at room temperature under air atmosphere. The reaction was quenched with sat. NaHCO₃(aq.) at 0 degrees C. The resulting mixture was washed with 3×20 mL of CH₂Cl₂. The product in the water layer. The water layer was concentrated under reduced pressure. The product was purified by Prep-SFC with the following conditions (Prep SFC80-2): Column, Green Sep Basic, 3*15 cm; mobile phase, CO₂ (70%) and IPA (0.5% 2M NH₃—MeOH) (30%); Detector, UV 254 nm; product was obtained. This resulted in 870 mg (57.89%) of 10 as a white solid. LC-MS: (ES, m/z): 351 [M+Na]+; 1H-NMR−: (300 MHz, DMSO-d₆) δ 11.28 (s, 1H), 7.56 (d, J=8.1 Hz, 1H), 5.86 (d, J=4.4 Hz, 1H), 5.65 (d, J=1.6 Hz, 1H), 5.56 (d, J=8.1 Hz, 1H), 4.17 (d, J=10.1 Hz, 1H), 4.10 (d, J=4.3 Hz, 1H), 4.00 (dd, J=10.1, 3.9 Hz, 1H), 3.87 (dt, J=4.1, 1.3 Hz, 1H), 3.72-3.49 (m, 8H), 2.08 (dd, J=7.1, 2.8 Hz, 1H), 2.05-1.96 (m, 1H).

### Preparation of 11

Into a 250 mL 3-necked round-bottom flask were added Molecularsieve and ACN (30.00 mL) at room temperature. The resulting mixture was stirred for 10 min at room temperature under argon atmosphere. Then to the stirred solution were added 3-[[bis(diisopropylamino)phosphanyl]oxy]propanenitrile (1058.46 mg, 3.512 mmol, 1.5 equiv) and DCI (359.12 mg, 3.043 mmol, 1.30 equiv). Then the dimethyl 10 (820.00 mg, 2.341 mmol, 1.00 equiv) in 30 mL ACN was added dropwise at room temperature under argon atmosphere. The resulting mixture was stirred for 1 h at room temperature under argon atmosphere. The resulting mixture was diluted with CH₂Cl₂ (60 mL). The combined organic layers were washed with water (3×40 mL) after filtration, dried over anhydrous MgSO4. After filtration, the filtrate was concentrated under reduced pressure. The residue was purified by Prep-TLC (0.5% TEA in PE/10% EtOH in EtOAc 1:9) to afford 11 (800 mg, 62.1%) as a colorless oil. LC-MS: (ES, m/z): 549 [M−H]⁻; 1H-NMR: (300 MHz, DMSO-d₆) δ 11.34 (s, 1H), 7.61 (dd, J=8.1, 1.7 Hz, 1H), 5.80 (dd, J=15.0, 1.8 Hz, 1H), 5.60 (d, J=8.1 Hz, 1H), 4.48-4.23 (m, 2H), 4.17-3.98 (m, 2H), 3.88-3.73 (m, 2H), 3.72-3.51 (m, 10H), 2.79 (q, J=5.9 Hz, 2H), 2.07 (dtt, J=17.9, 7.1, 3.2 Hz, 2H), 1.15 (ddd, J=6.3, 3.8, 2.1 Hz, 12H); ³¹P NMR (DMSO-d₆) δ 149.71, 149.35, 30.85, 30.75

### Example 45

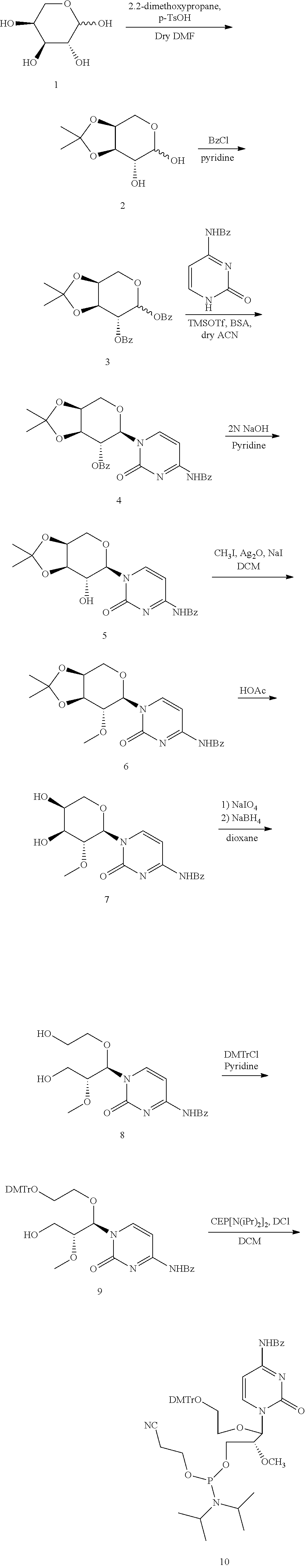

Preparation of 2: (J. Chem. Soc., Perkin Trans. 1, 1992, 1943-1952) To a solution of 1 (150.0 g, 1.0 mol) in DMF (2.0 L) was added 2, 2-dimethoxypropane (312.0 g, 3.0 mol) and p-TsOH (1.7 g, 10.0 mmol), then the reaction mixture was stirred at r.t. for 4 h, after the reaction, the solvent was concentrated to give the crude products which was used directly to next step.

Preparation of 3: (J. Chem. Soc., Perkin Trans. 1, 1992, 1943-1952) To a solution of 2 (190.0 g, 1.0 mol) in pyridine (2.0 L) was added BzCl (560.0 g, 4.0 mol) then the reaction mixture was stirred at r.t. for 2 h, after the reaction, the reaction mixture was poured into the ice water, EA was added for extraction, and the organic phase was washed with brine, dried over Na₂SO₄ and concentrated to give the crude product which was purified by silica gel column (EA:PE=1:5 to 1:1) to give 3 (350.0 g, 87.9% yield), ESI-LCMS: m/z=421.2 [M+Na]⁺.

Preparation of 4: (J. Chem. Soc., Perkin Trans. 1, 1992, 1943-1952) to a solution of 3 (240.0 g, 815.5 mmol) in MeCN (3.0 L) was added N-(2-oxo-1H-pyrimidin-4-yl) benzamide (193.0 g, 897.0 mmol) and BSA (496.6 g, 2.4 mol). then the reaction mixture was stirred at 50° C. for 30 min, then the reaction mixture was cooled to 0° C., and the TMSOTf (271.5 g, 1.2 mol) was added into the mixture at 0° C., then the reaction mixture was stirred at 70° C. for 2 h, after the reaction, the solvent was concentrated to give an oil, then the oil was poured into the solution of NaHCO₃ maintaining the mixture was slightly alkaline, EA was added for extraction, and the organic phase was washed with brine, dried over Na₂SO₄ and concentrated to give the crude product which was purified by silica gel column (EA:PE=1:3 to 1:1) to give 4 (180.0 g, 44.9% yield). ESI-LCMS: m/z=491.2 [M+H]⁺; ¹H NMR (400 MHz, DMSO-d₆) δ 11.19 (s, 1H), 8.20 (d, J=7.6 Hz, 1H), 8.01-7.84 (m, 4H), 7.73-7.57 (m, 2H), 7.50 (dt, J=10.4, 7.7 Hz, 4H), 7.40 (d, J=7.4 Hz, 1H), 6.03 (d, J=9.4 Hz, 1H), 5.33 (dd, J=9.4, 7.3 Hz, 1H), 4.66 (dd, J=7.3, 5.3 Hz, 1H), 4.45-4.35 (m, 2H), 4.22 (dd, J=13.7, 2.5 Hz, 1H), 1.58 (s, 3H), 1.34 (s, 3H).

Preparation of 5: To a solution of 4 (78.0 g, 158.7 mmol) in pyridine (800.0 mL) was added a solution of NaOH (6.3 g, 158.7 mmol) in a mixture solvent of H₂O and MeOH (4:1, 2N), Then the reaction mixture was stirred at 0° C. for 20 min, LC-MS and TLC show that the raw material was disappeared, then the mixture was pour into a solution of NH₄Cl, EA was added for extraction, and the organic phase was washed with brine, dried over Na₂SO₄ and concentrated to give the crude product, which was purified by silica gel column (DCM:MeOH=30:1 to 10:1) to give 5 (56.0 g, 91.0% yield). ESI-LCMS: m/z=388.1 [M+H]⁺; ¹H NMR (400 MHz, DMSO-d₆) δ 11.29 (s, 1H), 8.16 (d, J=7.6 Hz, 1H), 8.08-7.99 (m, 2H), 7.67-7.60 (m, 1H), 7.53 (t, J=7.6 Hz, 2H), 7.35 (d, J=7.6 Hz, 1H), 5.63 (d, J=6.1 Hz, 1H), 5.51 (d, J=9.5 Hz, 1H), 4.35-4.13 (m, 3H), 3.78 (dt, J=9.6, 6.5 Hz, 1H), 3.19 (d, J=5.1 Hz, 1H), 1.53 (s, 3H), 1.32 (s, 3H).

Preparation of 6: To a solution of 5 (15.0 g, 38.7 mmol) in DCM (200.0 mL) was added Ag₂O (35.8 g, 154.8 mmol), CH₃I (54.6 g, 387.2 mmol) and NaI (1.1 g, 7.7 mmol), then the reaction mixture was stirred at r.t. overnight, after the reaction, filtrate was obtained through filtration, and the filtrate concentrated the solvent to obtain the product 6 (13.0 g, 75.2% yield). ESI-LCMS: m/z=402.30 [M+H]⁺; ¹H NMR (400 MHz, DMSO-d₆) δ 11.30 (s, 1H), 8.22 (s, 1H), 8.00 (d, J=7.6 Hz, 2H), 7.71-7.20 (m, 4H), 5.56 (d, J=9.3 Hz, 1H), 4.33 (t, J=6.1 Hz, 1H), 4.26 (dd, J=6.2, 2.1 Hz, 1H), 4.20 (d, J=13.5 Hz, 1H), 3.98 (dd, J=13.5, 2.5 Hz, 1H), 3.66 (dd, J=9.3, 6.6 Hz, 1H), 3.34 (s, 3H), 1.57 (s, 3H), 1.32 (s, 3H).

Preparation of 7: To a solution of 6 (12.0 g, 29.9 mmol) was added CH₃COOH (120.0 mL), then the mixture was stirred at r.t. for 2 h, LC-MS and TLC showed that the raw material was disappeared, then the solvent was concentrated to get the crude product 7 (10.0 g, 83.3% yield). ESI-LCMS: m/z=362.1 [M+H]⁺.

Preparation of 8: To a solution of 7 (10.0 g, 24.9 mmol) in dioxane: H₂O=3:1 (120.0 mL) was added NaIO₄ (8.8 g, 41.5 mmol), then the reaction mixture was stirred at r.t. for 2 h, LC-MS and TLC showed that the raw material was disappeared, then the reaction mixture was cooled to 0° C., and NaBH₄ (2.4 g, 41.5 mmol) was added into the mixture and stirred at 0° C. for 0.5 h, LC-MS and TLC showed that the raw material was disappeared, then NH₄Cl was added into the mixture to adjust pH to be slightly alkaline, and concentrated to give the crude product, which was purified by silica gel column (PE:EA=5:1 to 1:1) to give 8 (8.0 g, 79.5% yield). ESI-LCMS: m/z=364.1 [M+H]⁺; ¹H NMR (400 MHz, DMSO-d₆) δ 11.26 (s, 1H), 8.14 (d, J=7.5 Hz, 1H), 8.07-7.94 (m, 2H), 7.67-7.59 (m, 1H), 7.52 (t, J=7.6 Hz, 2H), 7.37 (s, 1H), 5.91 (d, J=6.0 Hz, 1H), 4.77 (t, J=5.6 Hz, 1H), 4.70 (t, J=5.1 Hz, 1H), 3.70 (ddd, J=11.5, 5.0, 2.5 Hz, 1H), 3.57-3.39 (m, 6H), 3.31 (s, 3H).

Preparation of 9: To a solution of 8 (4.0 g, 11.0 mmol) in pyridine (50.0 mL) was added DMTrCl (5.5 g, 16.5 mmol), then the reaction mixture was stirred at r.t. for 2 h, LC-MS showed that the raw material was 20.0% and The ratio of product to by-product was 3.5:1. then the solvent was concentrated to get residue which was purified by silica gel column to give the purified products and by-products was 5 g in total, then the product was purified by SFC to get 9 (3.0 g, 40.9% yield). ESI-LCMS: m/z=666.2 [M+H]⁺; ¹H NMR (400 MHz, DMSO-d₆) δ 11.33 (s, 1H), 8.20 (d, J=7.4 Hz, 1H), 8.04 (d, J=7.7 Hz, 2H), 7.64 (t, J=7.4 Hz, 1H), 7.53 (t, J=7.6 Hz, 2H), 7.40 (d, J=7.8 Hz, 3H), 7.36-7.18 (m, 7H), 6.89 (d, J=8.4 Hz, 4H), 5.96 (d, J=5.7 Hz, 1H), 4.79 (t, J=5.7 Hz, 1H), 3.73 (s, 6H), 3.66-3.46 (m, 4H), 3.37 (s, 3H), 3.16 (ddd, J=10.1, 7.1, 3.0 Hz, 1H), 3.04 (dt, J=10.9, 3.4 Hz, 1H), 2.08 (s, 1H).

Preparation of 10: To a solution of 9 (2.8 g, 4.2 mmol) in DCM (30.0 mL) was added CEP[N(iPr)₂]₂ (1.3 g, 4.2 mmol) and DCI (601.2 mg, 5.1 mmol). The mixture was stirred at r.t. for 1 h. LC-MS showed 9 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=90/10; Detector, UV 254 nm. This resulted in to give 10 (2.8 g, 76.8% yield). ESI-LCMS: m/z=866.2 [M+H]⁺; ¹H NMR (400 MHz, DMSO-d₆) δ 11.34 (s, 1H), 8.22 (d, J=7.4 Hz, 1H), 8.09-7.98 (m, 2H), 7.64 (t, J=7.4 Hz, 1H), 7.53 (t, J=7.6 Hz, 2H), 7.45 (d, J=7.3 Hz, 1H), 7.39 (d, J=7.5 Hz, 2H), 7.31 (t, J=7.6 Hz, 2H), 7.24 (t, J=9.1 Hz, 5H), 6.89 (d, J=8.8 Hz, 4H), 5.96 (d, J=6.1 Hz, 1H), 4.02-3.86 (m, 1H), 3.84-3.63 (m, 11H), 3.56 (dtq, J=13.3, 6.6, 3.5, 3.1 Hz, 3H), 3.37 (s, 2H), 3.16 (ddd, J=10.0, 6.8, 3.3 Hz, 1H), 3.04 (ddd, J=10.7, 5.5, 3.0 Hz, 1H), 2.75 (td, J=5.9, 2.3 Hz, 2H), 1.18-1.07 (m, 12H); ³¹P NMR (DMSO-d₆) δ 148.02 (d, J=12.0 Hz).

### Example 46

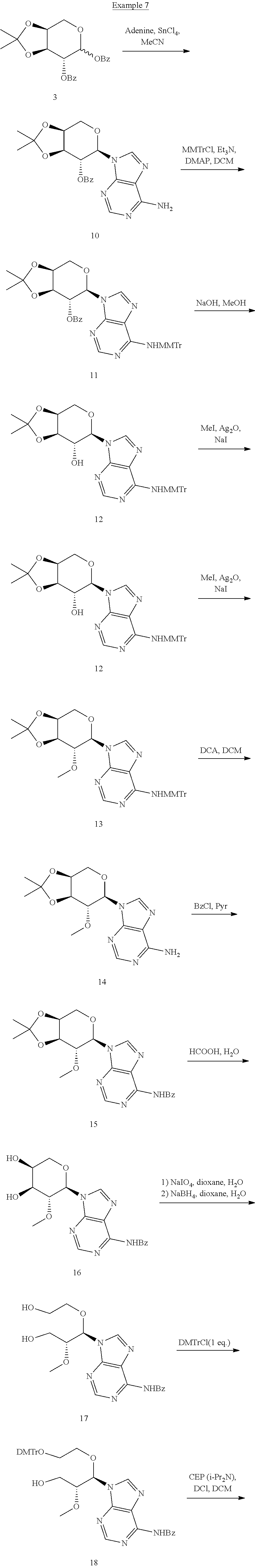

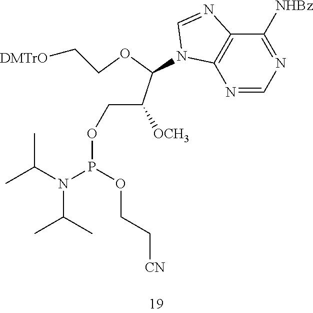

Preparation of 10: To the solution of 3 (200.0 g, 0.5 mol) in ACN (2000.0 mL) was added a solution of SnCl₄ in DCM (1000.0 mL) at 0° C. under N₂, and the reaction mixture was stirred at 0° C. for 4 h under N₂ atmosphere. Then the reaction solution was poured into saturated sodium bicarbonate solution, the resulting product was extracted with EA (3*500.0 mL). The combined organic layer was washed with water and brine, dried over Na₂SO₄, and concentrated to give the crude, which was purified by silica gel column (PE:EA=5:1 to 0:1) to give 10 (65.0 g, 31.4% yield) as a white solid. ESI-LCMS: m/z=412.0 [M+H]⁺; ¹H NMR (400 MHz, DMSO-d₆) δ 8.27 (s, 1H), 8.09 (s, 1H), 7.74-7.60 (m, 2H), 7.59-7.57 (m, 1H), 7.44-7.40 (m, 2H), 7.24 (s, 2H), 5.90 (d, J=9.6 Hz, 1H), 5.73 (dd, J=7.4 Hz, 1H), 4.63 (t, 1H), 4.50-4.30 (m, 2H), 4.21 (dd, J=13.6 Hz, 1H), 1.61 (s, 3H), 1.35 (s, 3H).

Preparation of 11: To a solution of 10 (40.0 g, 97.3 mmol) in DCM (500.0 mL) was added Et₃N (30.0 g, 297.0 mmol) and DMAP (1.2 g, 9.8 mmol) at r.t. The reaction mixture was replaced with N₂ over 3 times, then MMTrCl (45.0 g, 146.1 mmol) was added to the mixture. The reaction mixture was stirred at r.t. overnight. TLC and LC-MS showed that 10 was consumed, and the reaction mixture was added to an aqueous solution of NaHCO₃ in ice-water. Then extracted product with EA, washed the organic phase with brine, and dried the organic phase over Na₂SO₄, then concentrated to get 11 (66.5 g) as a crude, used next step directly.

Preparation of 12: To a solution of 11 (66.5 g, 97.3 mmol) in pyridine (600.0 mL) was added 2N NaOH (H₂O:MeOH=4:1) (200.0 mL) at r.t. Then the reaction mixture was stirred at 0° C. for 30 min, LC-MS and TLC showed that the raw material was disappeared, then the mixture was poured into a solution of NH₄Cl, EA was added for extraction, and the organic phase was washed with brine, dried over Na₂SO₄ and concentrated to give the crude product which was purified by silica gel column (EA:PE=1:5 to 1:1) to give 12 (50.0 g, 88.7% yield for two step). ESI-LCMS: m/z=580.4 [M+H]⁺; ¹H NMR (400 MHz, DMSO-d₆) δ 8.44 (s, 1H), 7.92 (s, 1H), 7.36-7.16 (m, 13H), 6.89-6.80 (m, 2H), 5.59 (d, J=6.0 Hz, 1H), 5.35 (d, J=9.6 Hz, 1H), 4.32-4.12 (m, 4H), 4.08-3.95 (m, 3H), 3.72 (s, 3H), 1.99 (s, 3H), 1.54 (s, 3H), 1.32 (s, 3H), 1.17 (t, J=7.1 Hz, 3H).

Preparation of 13: To a solution of 12 (46.0 g, 79.4 mmol) in CH₃I (200.0 mL) was added Ag₂O (36.6 g, 158.4 mmol) and NaI (6.0 g, 42.5 mmol), then the reaction mixture was stirred at r.t. for 4 h, then the reaction mixture was filtrated and concentrated the solvent to obtain the product 13 (46.0 g, 97.6% yield), used next step directly. ESI-LCMS: m/z=594.3 [M+H]⁺.

Preparation of 14: To a stirred solution of DCA (22.5 mL) in DCM (750.0 mL) was added 13 (46.0 g, 77.5 mmol) and Et₃Si (185.0 mL) at r.t. And the reaction mixture was stirred at r.t. for 12 h. The reaction solution was evaporated to dryness under reduced pressure to give a residue, which was slurry with a solution of NaHCO₃ (50.0 mL) to get 14 (19.0 g, 76% yield), which was used next step directly.

Preparation of 15: To a solution of 14 (16.0 g, 49.7 mmol) in pyridine (200.0 mL) was added BzCl (9.0 g, 64.7 mmol) at 0° C. Then the reaction mixture was stirred at r.t. for 2 h. LC-MS showed 6 was consumed completely, then the mixture was cooled to 0° C., and a solution of NaOH in MeOH and H₂O (2 N, 50.0 mL) was added into the reaction mixture, and the mixture was stirred for 1 h at 0° C., then the mixture was poured into a solution of NH₄Cl. The product was extracted with EA (300.0 mL) and the organic layer was washed with brine and dried over Na₂SO₄. Then the organic layer was concentrated to give a residue, which was purified by slurry with PE:EA (8:1, 900.0 mL) to get 15 (20.0 g, 95.0% yield). ESI-LCMS: m/z=426.2 [M+H]⁺; ¹H NMR (400 MHz, DMSO-d₆) δ 11.21 (s, 1H), 8.77-8.69 (m, 2H), 8.06 (d, J=7.6 Hz, 2H), 7.65 (t, J=7.4 Hz, 1H), 7.56 (t, J=7.6 Hz, 2H), 7.34-7.23 (m, 4H), 7.23-7.12 (m, 5H), 6.89-6.80 (m, 4H), 5.90 (d, J=7.9 Hz, 1H), 4.36-4.29 (m, 1H), 4.06 (t, J=8.8 Hz, 1H), 3.92 (dd, J=25.0, 6.9 Hz, OH), 3.72 (d, J=1.0 Hz, 7H), 3.59 (dt, J=10.4, 6.6 Hz, 1H), 3.24 (s, 3H), 2.97 (d, J=7.7 Hz, 1H), 2.76 (q, J=5.5 Hz, 2H), 1.14 (dd, J=9.2, 5.7 Hz, 12H).

Preparation of 16: To a mixture solution of HCOOH (180.0 mL) and H₂O (20.0 mL) was added 15 (19.0 g, 44.7 mmol). The reaction mixture was stirred at r.t. for 4 h. LC-MS showed 15 was consumed completely. Then the reaction mixture was concentrated to give a residue which was purified by slurry with MeOH (100.0 mL) to get 16 (16.0 g, 92.7% yield) as a white solid. ESI-LCMS: m/z=385.9 [M+H]⁺; ¹H NMR (400 MHz, DMSO-d₆) δ 11.21 (s, 1H), 8.77 (d, J=1.2 Hz, 2H), 8.09-8.02 (m, 2H), 7.70-7.61 (m, 1H), 7.56 (t, J=7.6 Hz, 2H), 5.56 (d, J=9.2 Hz, 1H), 5.21 (d, J=6.1 Hz, 1H), 4.94 (d, J=4.5 Hz, 1H), 4.18 (t, J=9.1 Hz, 1H), 4.09 (q, J=5.2 Hz, 1H), 3.88-3.71 (m, 4H), 3.21-3.14 (m, 6H).

Preparation of 17: To a solution of 16 (16.0 g, 41.4 mmol) in dioxane (200.0 mL) was added H₂O (32.0 mL), and NaIO₄ (9.7 g, 45.5 mmol), then the reaction mixture was stirred at r.t. for 1 h, LC-MS and TLC showed that the raw material was disappeared, then the reaction mixture was cooled to 0° C., and NaBH₄ (1.7 g, 45.5 mmol) was added into the mixture and stirred at 0° C. for 0.5 h, LC-MS and TLC showed that the intermediate state was disappeared, then the NH₄Cl was added into the mixture to adjust pH to be slightly alkaline, and concentrated at r.t. to give the crude product which was purified by silica gel column (DCM:MeOH=20:1 to 8:1) to give 17 (16.0 g, 99.5% yield). ESI-LCMS: m/z=388.0 [M+H]⁺; ¹H NMR (400 MHz, DMSO-d₆) δ 11.18 (s, 1H), 8.75 (s, 1H), 8.67 (s, 1H), 8.09-7.99 (m, 2H), 7.65 (t, J=7.4 Hz, 1H), 7.56 (t, J=7.6 Hz, 2H), 5.90 (d, J=7.6 Hz, 1H), 4.88 (t, J=5.7 Hz, 1H), 4.67 (t, J=5.5 Hz, 1H), 4.08-3.98 (m, 2H), 3.78 (ddd, J=12.1, 5.2, 3.1 Hz, 1H), 3.68-3.39 (m, 4H), 3.36 (s, OH), 3.20 (s, 3H), 1.99 (s, 1H), 1.17 (t, J=7.1 Hz, 1H).

Preparation of 18: To a solution of 17 (12.0 g, 31.0 mmol) in pyridine (50.0 mL) was added DMTrCl (11.5 g, 34.1 mmol), then the reaction mixture was stirred at r.t. for 2 h, LC-MS showed that the raw material was 15.0% remained and the ratio of product to by-product was 3.5:1. Then the reaction solution was poured into ice-water, and extracted with EA, wished with brine, dried over Na₂SO₄, filtered and concentrated to get residue which was purified by silica gel column to give the purified product and by-product were 13.0 g in total, then 4.0 g crude was purified by SFC to get 18 (3.3 g, 15.4% yield). ESI-LCMS: m/z=690.3 [M+H]⁺; ¹H NMR (400 MHz, DMSO-d₆) δ 11.21 (s, 1H), 8.75 (s, 1H), 8.69 (s, 1H), 8.10-8.03 (m, 2H), 7.70-7.61 (m, 1H), 7.56 (t, J=7.6 Hz, 2H), 7.35-7.12 (m, 9H), 6.90-6.80 (m, 4H), 5.94 (d, J=7.5 Hz, 1H), 4.88 (t, J=5.6 Hz, 1H), 4.36 (t, J=5.1 Hz, 1H), 4.11 (dt, J=7.4, 3.6 Hz, 1H), 3.82 (ddd, J=11.9, 5.1, 3.1 Hz, 1H), 3.72 (d, J=1.3 Hz, 7H), 3.64 (ddd, J=11.9, 6.2, 4.2 Hz, 1H), 3.45 (qd, J=7.0, 4.9 Hz, 2H), 3.24 (s, 3H), 3.09 (ddd, J=9.9, 6.4, 3.2 Hz, 1H), 2.97 (ddd, J=9.9, 5.7, 3.2 Hz, 1H), 1.23 (s, OH), 1.06 (t, J=7.0 Hz, 1H).

Preparation of 19: To a suspension of 18 (3.3 g, 4.8 mmol) in DCM (40.0 mL) was added DCI (0.5 g, 4.0 mmol) and CEP[N(iPr)₂]₂ (1.6 g, 5.3 mmol). The mixture was stirred at r.t. for 0.5 h. LC-MS showed 10 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 19 (3.0 g, 3.9 mmol, 81.2% yield) as a white solid. ESI-LCMS: m/z=765.3 [M+H]⁺; ¹H NMR (400 MHz, DMSO-d₆) δ 11.22 (s, 1H), 8.80-8.71 (m, 2H), 8.11-8.04 (m, 2H), 7.65 (t, J=7.3 Hz, 1H), 7.56 (t, J=7.5 Hz, 2H), 7.36-7.24 (m, 4H), 7.24-7.15 (m, 5H), 6.89-6.82 (m, 4H), 5.92 (d, J=7.7 Hz, 1H), 4.34 (dt, J=7.5, 3.5 Hz, 1H), 4.08 (ddd, J=10.7, 7.3, 2.7 Hz, 1H), 4.03-3.89 (m, 1H), 3.80-3.72 (m, 10H), 3.67-3.53 (m, 2H), 3.47 (dp, J=10.5, 3.4 Hz, 1H), 3.26 (s, 3H) 3.11 (ddd, J=10.3, 6.2, 3.5 Hz, 1H), 3.00 (q, J=6.6, 5.2 Hz, 1H), 2.77 (q, J=5.6 Hz, 2H), 2.08 (s, 1H), 1.15 (t, J=7.0 Hz, 12H); ³¹P NMR (162 MHz, DMSO-d₆) δ 148.30, 147.99.

### Example 47

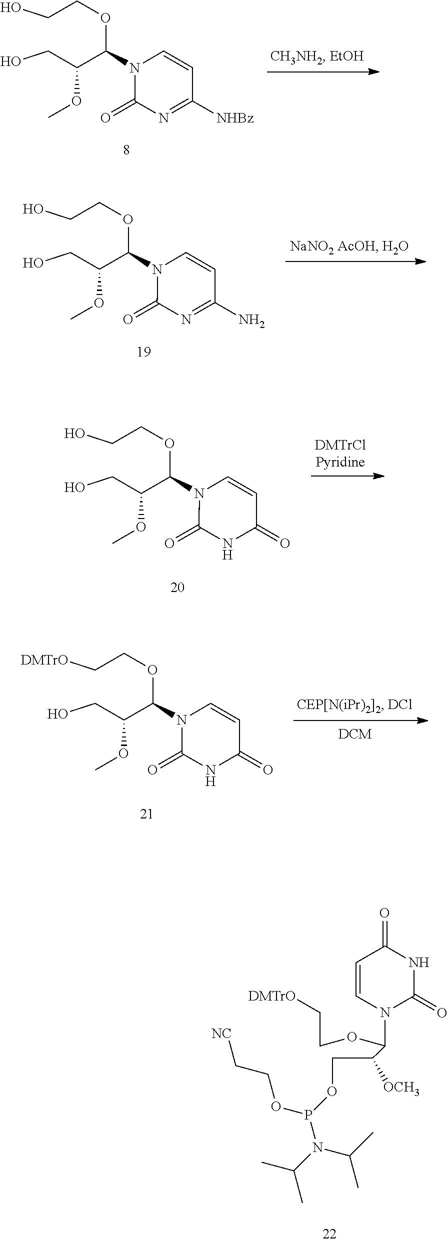

Preparation of 19: To a solution of 8 (8.0 g, 22.0 mmol) in EtOH (50.0 mL) was added a solution of CH₃NH₂ (50.0 mL), then the reaction mixture was stirred at r.t. for 4 h, after the reaction, the solvent was concentrated to give the crude, which was added into a mixture solvent of EA (20.0 mL) and PE (10.0 mL), then the mixture was stirred for 30 min and filtered to get 19 (5.5 g, 96.5% yield), which was used directly to next step.

Preparation of 20: (J. Chem. Soc., Perkin Trans. 1, 1992, 1943-1952) To a solution of 19 (5.0 g, 19.3 mmol) in H₂O (50.0 mL) and AcOH (50.0 mL) was added NaNO₂ (65.0 g, 772.0 mmol), then the reaction mixture was stirred at r.t. for 2 h, after the reaction, the reaction mixture was concentrated to give the crude product which was purified by silica gel column (DCM:MeOH=20:1 to 6:1) and MPLC (ACN: H₂O=0:100 to 10:90) to give 20 (3.0 g, 59.6% yield). ESI-LCMS: m/z=261.2 (M+H)⁺; ¹H NMR (400 MHz, DMSO-d₆) δ 11.29 (s, 1H), 7.66 (d, J=8.0 Hz, 1H), 5.67 (dd, J=17.5, 7.6 Hz, 2H), 4.74 (d, J=36.0 Hz, 2H), 3.86-3.63 (m, 1H), 3.58-3.40 (m, 6H).

Preparation of 21: To a solution of 20 (3.0 g, 11.5 mmol) in pyridine (30.0 mL) was added DMTrCl (3.9 g, 11.5 mmol), then the reaction mixture was stirred at r.t. for 2 h, LC-MS showed that the raw material was 20.0% and The ratio of product to by-product was 3:1, then the mixture was poured into a solution of NaHCO₃ (100.0 mL), and extracted with EA (100.0 mL), washed with brine and dried over Na₂SO₄, filtered and concentrated to get residue, which was purified by silica gel column to give The purified products and by-products were 5.0 g in total, then the product was purified by SFC to give 21 (1.8 g). ESI-LCMS: m/z=561.2 [M+H]⁺; ¹H NMR (400 MHz, DMSO-d₆) δ 11.31 (s, 1H), 7.69 (d, J=8.1 Hz, 1H), 7.45-7.15 (m, 8H), 6.88 (d, J=8.5 Hz, 4H), 5.71 (d, J=6.8 Hz, 1H), 5.64 (d, J=8.0 Hz, 1H), 4.79 (t, J=5.5 Hz, 1H), 3.74 (s, 6H), 3.60 (s, 1H), 3.51 (d, J=5.5 Hz, 3H), 3.11 (d, J=6.7 Hz, 1H), 3.02 (d, J=7.0 Hz, 1H).

Preparation of 22: To a solution of 21 (1.8 g, 3.2 mmol) in DCM (20.0 mL) was added CEP[N(iPr)₂]₂ (1.0 g, 3.4 mmol) and DCI (321.0 mg, 2.7 mmol). The mixture was stirred at r.t. for 1 h. LC-MS showed 21 was consumed completely. The solution was washed with solution of NaHCO₃ twice and washed with brine and dried over Na₂SO₄. Then concentrated to give a residue, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=90/10; Detector, UV 254 nm. This resulted in to give 22 (2.0 g, 82% yield). ESI-LCMS: m/z=761.2 [M+H]⁺; ¹H NMR (400 MHz, DMSO-d₆) δ 11.35 (s, 1H), 7.73 (dd, J=8.0, 2.0 Hz, 1H), 7.39 (d, J=7.4 Hz, 2H), 7.35-7.18 (m, 7H), 6.94-6.82 (m, 4H), 5.81-5.74 (m, 1H), 5.67 (d, J=8.0 Hz, 1H), 4.11-3.85 (m, 1H), 3.82-3.67 (m, 11H), 3.67-3.50 (m, 5H), 3.17-3.09 (m, 1H), 3.09-3.01 (m, 1H), 2.74 (td, J=5.8, 2.9 Hz, 2H), 1.13 (dd, J=9.2, 6.7 Hz, 13H); ³¹P NMR (DMSO-d₆) δ 148.09 (d, J=41.8 Hz). Example 48

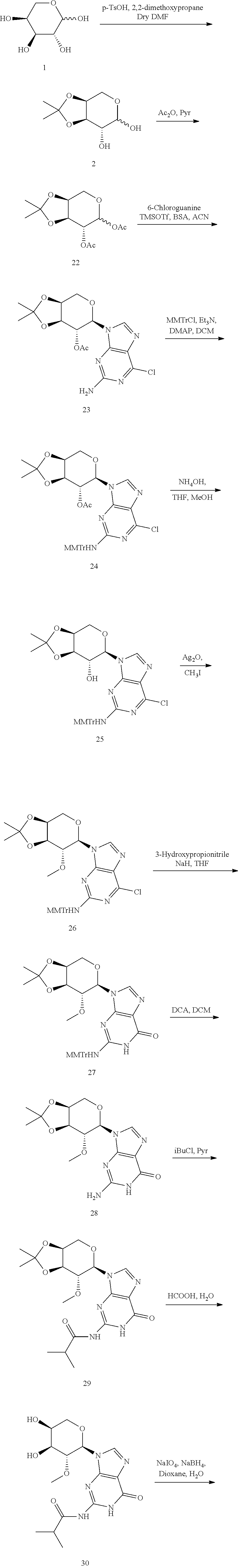

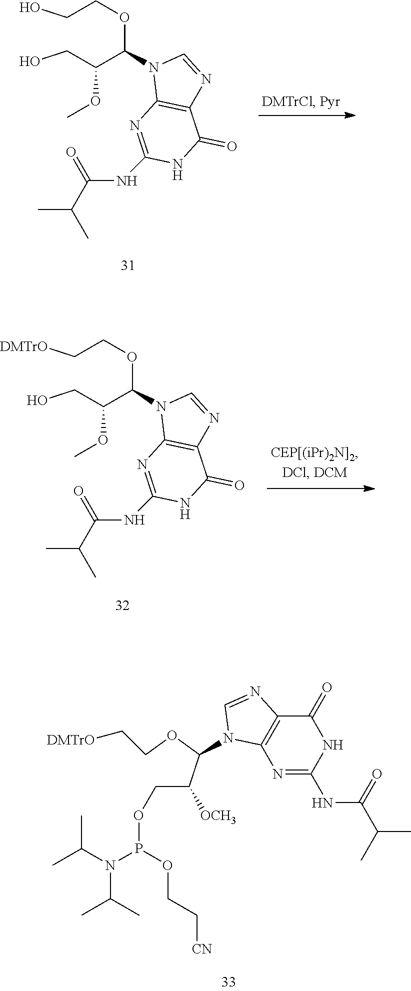

Preparation of 2 (J. Chem. Soc., Perkin Trans. 1, 1992, 1943-1952): To a solution of 1 (150.0 g, 999.1 mmol) in DMF (1000.0 mL) was added P-TsOH (1.7 g, 10.0 mmol), then 2,2-dimethoxy-propane (312.2 g, 3.0 mol) was added to the reaction mixture. The reaction mixture was stirred for 5 h at r.t. 90.0% 1 was consumed by TLC. Then NaHCO₃ (8.4 g, 99.9 mmol) was added to the reaction mixture, filtered out the solid after 30 min, and concentrated the organic phase by vacuum to obtain crude, which was purified by c.c. (PE:EA=1:1 to 0:1) to get compound 2 (115.0 g, 60.5% yield) as a white solid.

Preparation of 22: A solution of 2 (115.0 g, 604.6 mmol) in pyridine (600.0 mL) was cooled to 0° C., then Ac₂O (185.2 g, 1.81 mol) was added drop wise to the reaction mixture. The reaction was stirred for 2 h at r.t., and the raw material was consumed by TLC. The reaction solution was added into water, extracted product with EA. The organic phase was washed with brine, and dried the organic phase with Na₂SO₄, and concentrated to get 22 (150.0 g, 90.4% yield), which was used for next step directly. ¹H NMR (400 MHz, Chloroform-d) δ 6.20 (d, J=3.4 Hz, 1H), 5.66 (d, J=6.8 Hz, 1H), 5.17 (t, J=6.9 Hz, 1H), 5.10 (dd, J=7.0, 3.4 Hz, 1H), 4.40-4.25 (m, 3H), 4.21 (dd, J=7.0, 6.1 Hz, 1H), 4.16-4.02 (m, 3H), 3.95 (dd, J=12.9, 4.4 Hz, 1H), 2.17 (s, 1H), 2.15-2.03 (m, 12H), 1.56 (d, J=4.0 Hz, 6H), 1.37 (d, J=3.1 Hz, 6H).

Preparation of 23: To a solution of 22 (150.0 g, 546.9 mmol) in ACN (2200.0 mL) was added 6-chloroguanine (139.1 g, 820.4 mmol) and BSA (333.7 g, 1.6 mol) at r.t., then the reaction mixture was replaced with N₂ over 3 times. The reaction was stirred for 30 min at 50° C. After that, the reaction mixture was cooled to 0° C. under N₂. Then TMSOTf (182.1 g, 820.4 mmol) was added into the mixture. After addition, the reaction was stirred for 1.5 h at 70° C. TLC and LC-MS showed the raw material was consumed. Concentrated the most organic solvent by vacuum, then the residual was added to an aqueous solution of NaHCO₃ in ice-water, extracted product with EA (4.0 L), dried the organic phase over Na₂SO₄, and filtered and concentrated to get crude, which was purified by c.c. (DCM to DCM:EA=5:1) to get compound 23 (82.0 g, 35.0% yield) as a white solid. ESI-LCMS: m/z=384.8 [M+H]⁺; ¹H NMR (400 MHz, DMSO-d₆) δ 8.23 (s, 1H), 7.04 (d, J=22.3 Hz, 2H), 5.57 (d, J=9.6 Hz, 1H), 5.40 (dd, J=9.6, 7.3 Hz, 1H), 4.48 (dd, J=7.4, 5.4 Hz, 1H), 4.40-4.30 (m, 2H), 4.11 (dd, J=13.6, 2.4 Hz, 1H), 1.81 (s, 3H), 1.55 (s, 3H), 1.34 (s, 3H).

Preparation of 24: To a solution of 23 (82.0 g, 192.3 mmol) in DCM (1000.0 mL) was added Et₃N (59.4 g, 576.9 mmol) and DMAP (2.4 g, 19.2 mmol) at r.t. The reaction mixture was replaced with N₂ over 3 times, then MMTrCl (90.9 g, 288.4 mmol) was added into the mixture. The reaction mixture was stirred at r.t. overnight. TLC and LC-MS showed that 92.0% raw material was consumed, and the reaction mixture was added to an aqueous solution of NaHCO₃ in ice-water, then extracted product with EA. Washed the organic phase with brine, and dried the organic phase over Na₂SO₄, then concentrated to get crude, which was purified by c.c. (DCM) to give compound 24 (110.0 g, 86.4% yield) as a white solid. ESI-LCMS: m/z=657.1 [M+H]⁺; ¹H NMR (400 MHz, DMSO-d₆) δ 8.21 (s, 1H), 7.37-7.31 (m, 4H), 7.29-7.23 (m, 6H), 7.20-7.15 (m, 2H), 6.86-6.80 (m, 2H), 5.75 (s, 1H), 5.23 (dd, J=9.6, 7.2 Hz, 1H), 4.85 (s, 1H), 4.44-4.16 (m, 3H), 3.71 (s, 4H), 1.70 (s, 3H), 1.49 (s, 3H), 1.31 (s, 3H).

Preparation of 25: To a solution of 24 (110.0 g, 164.3 mmol) in a mixed solvent of THF (500.0 mL) and MeOH (160.0 mL) was added NH₄OH (330.0 mL). The reaction mixture was stirred overnight at r.t., and the raw material was consumed by TLC and LC-MS. The reaction liquid was added into water, extracted product with EA. Washed the organic phase with brine, then dried the organic phase over Na₂SO₄, then concentrated to get the crude, which was purified by c.c. (PE:EA=10:1-1:2) to give compound 25 (98.0 g, 94.2% yield) as a white solid. ESI-LCMS: m/z=615.1 [M+H]⁺; ¹H NMR (400 MHz, DMSO-d₆) δ 8.32 (s, 1H), 7.36 (dt, J=8.2, 1.4 Hz, 4H), 7.31-7.21 (m, 6H), 7.15 (t, J=7.2 Hz, 2H), 6.85-6.76 (m, 2H), 5.57 (d, J=4.6 Hz, 1H), 4.69 (s, 1H), 4.25 (dt, J=5.1, 2.4 Hz, 1H), 4.03 (q, J=7.1 Hz, 4H), 3.70 (s, 3H), 3.62-3.44 (m, 1H), 1.51 (s, 3H), 1.31 (s, 3H).

Preparation of 26 (Ref WO2011/95576, 2011, A1): To a solution of 25 (70.0 g, 114.0 mmol) in CH₃I (350.0 mL) was added Ag₂O (79.2 g, 342.0 mmol) at r.t. Then the reaction mixture was stirred for 4 h at r.t. TLC and LC-MS showed that the raw material was consumed. Filtered out the residue with diatomite, and concentrated the filtrate by vacuum to get crude, which was purified by c.c. (PE:EA=10:1-1:1) to get compound 26 (28.0 g, 31.3% yield) as a white solid. ESI-LCMS: m/z=629.1 [M+H]⁺.

Preparation of 27: A solution of 3-hydroxy-propionitrile (15.6 g, 219.7 mmol) in THF (200.0 mL) was cooled to 0° C. The reaction mixture was replaced by N₂ over 3 times. Then NaH (12.4 g, 310.0 mmol, 60.0%) was added to the reaction mixture in turn. The reaction was stirred for 30 min at r.t., and then the reaction was cooled to 0° C. again. A solution of 26 (26.0 g, 33.0 mmol) in THF (150.0 mL) was added drop wise to the reaction mixture. Then the reaction mixture was stirred at r.t. overnight. TLC and LC-MS showed the raw material was consumed. The reaction liquid was added into water, extracted product with EA. The organic phase was washed with brine, and dried over Na₂SO₄, then concentrated to get the crude, which was purified by c.c. (DCM:MeOH=50:1-30:1) to get compound 27 (18.0 g, 88.0% yield) as white solid. ESI-LCMS: m/z=610.7 [M+H]⁺; ¹H NMR (400 MHz, DMSO-d₆) δ 10.68 (s, 1H), 7.90 (s, 1H), 7.69 (s, 1H), 7.34-7.15 (m, 12H), 6.92-6.81 (m, 2H), 4.46 (d, J=9.5 Hz, 1H), 4.22 (dt, J=5.5, 2.5 Hz, 1H), 4.07 (t, J=6.4 Hz, 1H), 3.84 (dd, J=13.5, 2.1 Hz, 1H), 3.64-3.54 (m, 1H), 3.36 (dd, J=13.3, 2.8 Hz, 1H), 3.08 (s, 3H), 2.59 (t, J=6.0 Hz, 3H), 1.49 (s, 3H), 1.30 (s, 3H).

Preparation of 28 (Beigelman, Leonid; Deval, Jerome; Jin, Zhinan WO2014/209979, 2014, A1): To a solution of 27 (18.0 g, 29.5 mmol) in DCM (300.0 mL) was added triethylsilane (70.0 mL) and DCA (10.0 mL) at r.t. Then the reaction mixture was stirred for 6 h at r.t., TLC and LC-MS showed that the raw material was consumed. Concentrated the almost organic solvent by vacuum, then PE (600.0 mL) was added to the reaction mixture. Filtered of the organic phase to get the solid, which was purified by MPLC (MeCN: H₂O=40:60 to 50:50) to get compound 28 (7.5 g, 75.0% yield) as a white solid. ESI-LCMS: m/z=338.3 [M+H]⁺; ¹H NMR (400 MHz, DMSO-d₆) δ 10.70 (s, 1H), 8.03 (s, 1H), 6.49 (s, 2H), 5.15 (d, J=9.6 Hz, 1H), 4.28 (d, J=5.1 Hz, 2H), 4.20 (d, J=13.6 Hz, 1H), 3.93 (ddd, J=13.3, 10.6, 3.7 Hz, 2H), 3.26 (s, 3H), 1.59 (s, 3H), 1.33 (s, 3H);

Preparation of 29: A solution of 28 (7.0 g, 20.6 mmol) in Pyr (150.0 mL) was cooled to 0° C. Then the reaction mixture was added i-BuCl (6.6 g, 61.8 mmol) drop wise. The reaction mixture was stirred for 30 min, TLC and LC-MS showed the raw material was consumed. The reaction liquid was added to ice-water, extracted product with EA. The organic phase was washed with brine, and dried over Na₂SO₄, and filtered and concentrated to get the crude, which was purified by c.c. (DCM:MeOH=100:1-30:1) to get compound 29 (5.8 g, 68.6% yield) as a white solid. ESI-LCMS: m/z=409.4 [M+H]⁺; ¹H NMR (400 MHz, DMSO-d₆) δ 12.13 (s, 1H), 11.66 (s, 1H), 8.39 (s, 1H), 5.24 (d, J=9.6 Hz, 1H), 4.36-4.23 (m, 3H), 3.99-3.88 (m, 2H), 3.27 (s, 4H), 2.78 (hept, J=6.8 Hz, 1H), 1.61 (s, 3H), 1.35 (s, 3H), 1.12 (d, J=6.8 Hz, 6H).

Preparation of 30: A solution of 29 (5.8 g, 14.1 mmol) was added into a mixed solvent of HCOOH (54.0 mL) and H₂O (6.0 mL) at r.t. Then reaction mixture was stirred for 1 h at r.t. TLC and LC-MS showed the raw material was consumed. Concentrated the reaction solution by vacuum at r.t. to get compound 30 (5.2 g, 14.0 mmol, 98.0% yield), which was used for next step directly. ESI-LCMS: m/z=368.4 [M+H]⁺; ¹H NMR (400 MHz, DMSO-d₆) δ 12.13 (s, 1H), 11.72 (s, 1H), 8.30 (s, 1H), 8.14 (s, 2H), 5.19 (d, J=9.2 Hz, 1H), 3.93 (t, J=9.2 Hz, 1H), 3.85 (dd, J=12.4, 1.9 Hz, 1H), 3.77 (d, J=3.7 Hz, 1H), 3.69-3.62 (m, 2H), 3.20 (s, 3H), 2.79 (h, J=6.8 Hz, 1H), 1.13 (dd, J=6.9, 1.2 Hz, 6H).

Preparation of 31: To a solution of 30 (5.2 g, 14.0 mmol) in dioxane (90.0 mL) and H₂O (30.0 mL) was added NaIO₄ (3.7 g, 15.4 mmol) at r.t. The reaction mixture was stirred for 3 h at r.t. LC-MS showed the raw material was consumed, and the reaction solution was cooled to 0° C. Then NaBH₄ (970.0 mg, 25.2 mmol) was added to the reaction mixture, and the raw material was consumed after 3 h by LC-MS. The reaction liquid was quenched with ammonium chloride, and adjusted the pH to 6-7 with 1N HCl, the mixture solution was concentrated to get the crude, which was purified by c.c. (DCM:MeOH=100:1-30:1) to get compound 31 (4.0 g, 68.6% yield) as a white solid. ESI-LCMS: m/z=370.4 [M+H]⁺; ¹H NMR (400 MHz, DMSO-d₆) δ 11.91 (d, J=151.0 Hz, 2H), 8.62-8.51 (m, 1H), 8.18 (s, 1H), 7.44-7.33 (m, 1H), 5.62 (d, J=7.9 Hz, 1H), 4.84 (t, J=5.7 Hz, 1H), 4.65 (d, J=5.2 Hz, 1H), 3.84 (dd, J=7.7, 3.5 Hz, 1H), 3.76 (ddd, J=12.1, 4.7, 2.7 Hz, 1H), 3.60 (ddd, J=12.0, 5.8, 3.6 Hz, 1H), 3.46 (d, J=8.8 Hz, 2H), 3.16 (s, 3H), 2.77 (h, J=6.8 Hz, 1H), 1.12 (dd, J=6.8, 2.4 Hz, 6H);

Preparation of 32: A solution of 31 (4.0 g, 6.4 mmol) was dissolved in pyridine (100.0 mL), and the reaction mixture was replaced by N₂ over 3 times, and then DMTrCl (5.1 g, 8.9 mmol) was added to the reaction mixture at r.t. Then the reaction was stirred for 30 min, TLC and LC-MS showed raw material was consumed. The reaction liquid was added into ice-water, and extracted product with EA. The organic phase was washed with brine, and dried the organic phase over Na₂SO₄, and concentrated to get crude, which was purified by c.c. (DCM:MeOH=100:1-30:1) and SFC to get compound 32 (2.7 g, 37.1% yield) as a white solid. ESI-LCMS: m/z=672.7 [M+H]⁺; ¹H NMR (400 MHz, DMSO-d₆) δ 11.50 (s, 2H), 8.22 (s, 1H), 7.32-7.24 (m, 4H), 7.22-7.12 (m, 5H), 6.84 (dd, J=9.0, 2.4 Hz, 4H), 5.63 (d, J=7.9 Hz, 1H), 4.85 (t, J=5.6 Hz, 1H), 3.95 (dt, J=7.4, 3.3 Hz, 1H), 3.85-3.77 (m, 1H), 3.73 (s, 7H), 3.65-3.57 (m, 1H), 3.43 (ddt, J=9.9, 6.9, 3.4 Hz, 1H), 3.05 (ddd, J=10.0, 6.2, 3.3 Hz, 1H), 2.96 (ddd, J=10.0, 5.6, 3.4 Hz, 1H), 2.78 (p, J=6.8 Hz, 1H), 1.11 (d, J=6.7 Hz, 6H).

Preparation of 33: To a solution of 32 (2.7 g, 2.4 mmol) in DCM (35.0 mL) was added DCI (390.0 mg, 2.0 mmol) at r.t. Then CEP[N(iPr)₂]₂ (1.2 g, 2.5 mmol) was added to the reaction mixture, then reaction mixture was stirred for 30 min at r.t. LC-MS showed raw material was consumed. The reaction liquid was added to an aqueous solution of NaHCO₃ into ice-water, and extracted product with DCM, washed the organic phase with brine, and dried the organic phase over Na₂SO₄, then filtered and concentrated to give a residue, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20.0 min, the eluted product was collected at CH₃CN/H₂O (0.5% NH₄HCO₃)=100/0; Detector, UV 254 nm. This resulted in to give compound 33 (2.0 g, 56.4% yield) as a white solid. ESI-LCMS: m/z=872.3 [M+H]⁺; ¹H NMR (400 MHz, DMSO-d₆) δ 11.79 (s, 2H), 8.23 (d, J=1.7 Hz, 1H), 7.35-7.07 (m, 9H), 6.92-6.75 (m, 4H), 5.52 (d, J=8.0 Hz, 1H), 4.21 (s, 1H), 4.10-3.99 (m, 1H), 3.84-3.65 (m, 10H), 3.63-3.52 (m, 2H), 3.45 (ddd, J=10.2, 6.7, 3.6 Hz, 1H), 3.34 (s, 1H), 3.22 (s, 3H), 3.07 (ddd, J=10.2, 6.4, 3.4 Hz, 1H), 2.97 (ddd, J=10.0, 5.6, 3.5 Hz, 1H), 2.78 (dt, J=12.2, 6.4 Hz, 3H), 1.20-1.05 (m, 18H), ³¹P NMR (162 MHz, DMSO-d₆) δ 148.20, 147.13.

### Example 49

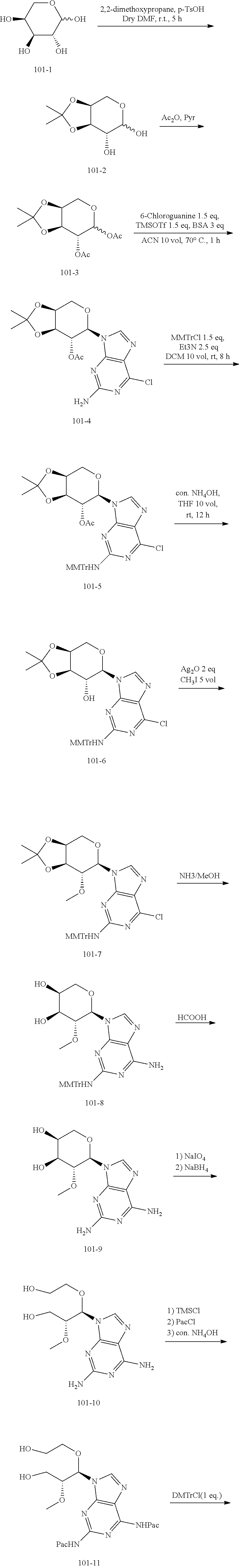

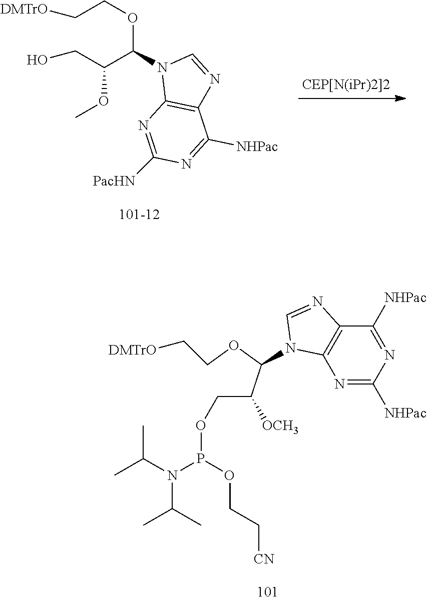

### Example 50

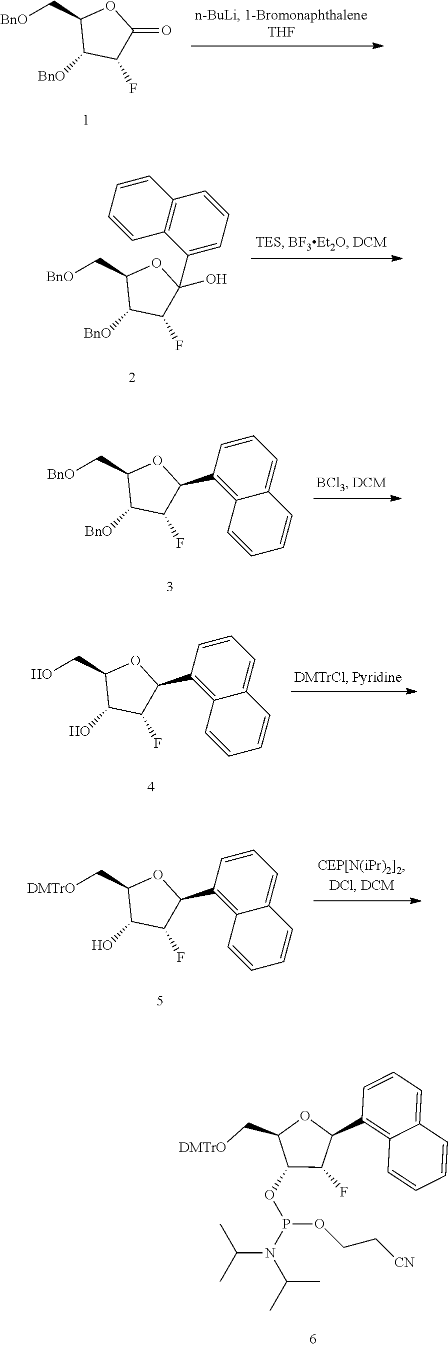

Preparation of 2: To a solution of 1-bromonaphthalene (5.2 g, 25.0 mmol) in dry THF (100.0 mL) was added n-BuLi (13.5 mL, 21.7 mmol, 1.6 M) drop wise at −78° C., then the mixture was stirred at −78° C. for 0.5 h, after that, a solution of 1 (5.5 g, 16.7 mmol) in THF (20.0 mL) was added into the mixture drop wise maintaining inner temperature below −70° C., then the reaction mixture was stirred for 1 h at −70° C. LC-MS showed 1 was consumed completely, the reaction was quenched with saturated ammonium chloride solution (80.0 mL) and extracted with EA, The organic layer was washed with brine, dried over Na₂SO₄, and concentrated under reduced pressure to give a residue, which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 2 (5.8 g, 76.3% yield) as a white solid. ESI-LCMS: m/z 441 [M−OH]⁻.

Preparation of 3: To the solution of 2 (5.8 g, 12.6 mmol) in DCM (100.0 mL) was added TES (1.7 g, 14.7 mmol) at −78° C., BF₃·Et₂O (2.7 g, 18.9 mmol) was added into the mixture drop-wise at −78° C. The mixture was stirred at −40° C. for 1 h. LC-MS showed 2 was consumed completely, the solution was added into a saturated sodium bicarbonate solution (50.0 mL) and extracted with DCM. The organic layer was washed with brine, dried over Na₂SO₄, and concentrated under reduced pressure to give a residue, which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in to give 3 (2.7 g, 48.2%) as a white solid. ESI-LCMS: m/z 460 [M+H₂O]⁺; ¹H-NMR (600 MHz, CDCl₃): δ 8.01-8.00 (d, J=6.5 Hz, 1H), 7.88-7.87 (d, J=7.6 Hz, 2H), 7.77-7.76 (d, J=8.2 Hz, 1H), 7.56-7.49 (m, 2H), 7.38-7.23 (m, 11H), 6.98-5.94 (d, J=26.9 Hz, 1H), 5.09-4.99 (dd, J=61.1 Hz, 1H), 4.71-4.69 (d, J=11.6 Hz, 1H), 4.66-4.59 (m, 2H), 4.43-4.41 (d, J=11.6 Hz, 2H), 4.14-4.08 (m, 1H), 4.02-4.00 (dd, J=13.4 Hz, 1H), 3.81-3.78 (dd, J=14.8 Hz, 1H); ¹⁹F-NMR (CDCl₃): δ −193.24.

Preparation of 4: To a solution of 3 (2.7 g, 6.0 mmol) in dry DCM (40.0 mL) was added BCl₃ (36.0 mL, 36.0 mmol, 1 M) drop wise at −78° C., and the reaction mixture was stirred at −78° C. for 0.5 h. LC-MS showed 3 was consumed completely. After completion of reaction, the resulting mixture was quenched with MeOH (20.0 mL), then neutralized with sodium hydroxide solution (40.0 mL, 2 M). The mixture was extracted with DCM and concentrated to give a crude, the crude was dissolved in MeOH (30.0 mL) and added a sodium hydroxide solution (30.0 mL, 4 M), and the mixture was stirred at r.t. for 30 min. The mixture was extracted with EA, the organic layer was washed with brine, dried over Na₂SO₄, and concentrated under reduced pressure to give a residue, which was purified by silica gel column chromatography (DCM:MeOH=40:1˜15:1) to give 4 (1.3 g, 81.2%) as a white solid. ESI-LCMS: m/z 261 [M−H]⁻; ¹H-NMR (DMSO-d₆): δ 7.98-7.97 (d, J=10.2 Hz, 2H), 7.89-7.87 (m, 2H), 7.63-7.49 (m, 3H), 5.80-5.76 (d, J=26.3 Hz, 1H), 5.43 (s, 1H), 5.00 (s, 1H), 4.85-4.76 (d, J=58.4 Hz, 1H), 4.03-3.85 (m, 3H), 3.68-3.66 (m, 1H), 3.65-3.53 (m, 1H); ¹⁹F-NMR (DMSO-d₆): δ −192.76.

Preparation of 5: To a solution of 4 (1.3 g, 5.0 mmol) in pyridine (20.0 mL) was added DMTrCl (6.1 g, 16.0 mmol) at r.t. The reaction mixture was stirred at r.t. for 1 h. The LC-MS showed 4 was consumed and water (100.0 mL) was added. The product was extracted with EA and the organic layer was washed with brine and dried over Na₂SO₄, concentrated to give the crude, which was further purified by silica gel (EA:PE=1:30˜1:10) to give 5 (2.2 g, 78.5%) as a yellow solid. ESI-LCMS: m/z 563 [M−H]⁻; ¹H-NMR (600 MHz, DMSO-d₆): δ 8.03-7.99 (m, 2H), 7.91-7.86 (m, 2H), 7.64-7.57 (m, 2H), 7.49-7.48 (d, J=6.8 Hz, 2H), 7.40-7.24 (m, 8H), 6.89-6.88 (m, 4H), 5.92-5.88 (d, J=26.6 Hz, 1H), 5.50-5.49 (d, J=4.5 Hz, 1H), 4.96-4.87 (d, J=56.2 Hz, 1H), 4.18-4.14 (m, 2H), 3.74 (s, 6H), 3.42-3.40 (d, J=9.9 Hz, 1H), 3.33 (m, 2H); ¹⁹F-NMR (DMSO-d₆): δ −192.18.

Preparation of 6: To a suspension of 5 (2.2 g, 3.9 mmol) in DCM (20.0 mL) was added DCI (391.0 mg, 3.3 mmol) and CEP[N(iPr)₂]₂ (1.4 g, 4.7 mmol). The mixture was stirred at r.t. for 1 h. The LC-MS showed 5 was consumed completely. The solution was washed with a saturated sodium bicarbonate solution and brine successively, dried over Na₂SO₄, concentrated to give the crude, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in to give 6 (2.5 g, 83.8%) as a white solid. ESI-LCMS: m/z 765 [M+H]⁺; ¹H-NMR (400 MHz, DMSO-d₆): δ 8.07-7.86 (m, 4H), 7.64-7.56 (m, 2H), 7.49-7.45 (m, 2H), 7.41-7.21 (m, 8H), 6.89-6.84 (m, 4H), 6.02-5.93 (m, 1H), 5.19-4.98 (m, 1H), 4.61-4.34 (m, 1H), 4.26-4.24 (m, 1H), 3.74-3.73 (m, 6H), 3.70-3.61 (m, 1H), 3.57-3.42 (m, 4H), 3.29-3.24 (m, 1H), 2.67-2.64 (m, 1H), 2.56-2.52 (m, 1H), 1.09-1.04 (m, 1H), 0.98-0.97 (d, J=6.7 Hz, 3H), 0.89-0.87 (d, J=6.7 Hz, 3H); ¹⁹F-NMR (DMSO-d₆): δ −191.75, −191.76, −191.84, −191.85; ³¹P-NMR (DMSO-d₆): δ 149.51, 149.47, 149.16, 149.14.

### Example 51

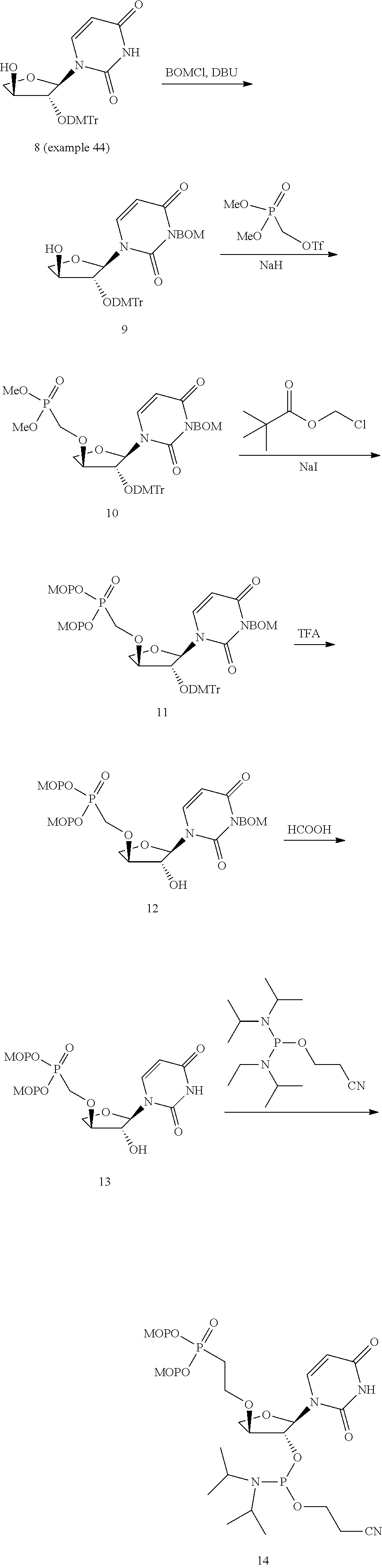

### Preparation of 9

To a solution of 8 (from Example 44) (6.6 g, 10.86 mmol, 85% purity, 1 eq) and DBU (3.31 g, 21.72 mmol, 3.27 mL, 2 eq) in DMF (70 mL) was added BOMCl (2.55 g, 16.29 mmol, 2.26 mL, 1.5 eq) at 0° C. The mixture was stirred at 20° C. for 12 h. The mixture was diluted with EtOAc (180 mL) and washed with H₂O (80 mL*3), and brine (80 mL), dried over Na₂SO₄, filtered and concentrated under reduced pressure to give a residue. The residue was purified by flash silica gel chromatography (ISCO®; 80 g SepaFlash® Silica Flash Column, Eluent of 10-60%, EtOAc/PE gradient @ 60 mL/min) to give 9 (5.2 g, 70% yield) as a white foam. LCMS (ESI): m/z 659.1; ¹H NMR (400 MHz, DMSO-d₆) δ=7.63 (d, J=8.3 Hz, 1H), 7.40-7.15 (m, 14H), 6.85 (t, J=8.0 Hz, 4H), 5.97 (s, 1H), 5.75 (d, J=8.0 Hz, 1H), 5.39-5.26 (m, 2H), 5.24 (d, J=2.0 Hz, 1H), 4.61 (s, 2H), 3.97 (s, 1H), 3.94-3.83 (m, 2H), 3.68 (d, J=10.0 Hz, 6H), 3.38 (s, 1H)

### Preparation of 10

To a solution of 9 (5.2 g, 8.17 mmol, 1 eq) and dimethoxyphosphorylmethyl trifluoromethanesulfonate (6.67 g, 24.50 mmol, 3 eq) in THF (50 mL) was added NaH (816.65 mg, 20.42 mmol, 60% purity, 2.5 eq) at −5° C. The mixture was stirred at 0° C. for 0.5 h. The reaction mixture was quenched by addition H₂O (50 mL) and diluted with EtOAc (100 mL), then washed with H₂O (50 mL), brine (50 mL), the organic layer was dried over Na₂SO₄, filtered and concentrated under reduced pressure. The residue was purified by flash silica gel chromatography (ISCO®; 80 g SepaFlash® Silica Flash Column, Eluent of 0-50%, EtOAc/DCM gradient @ 60 mL/min) to give 10 (4.2 g, 66.42% yield) as a white foam. LCMS (ESI): m/z 781.1 [M+Na]⁺, ¹H NMR (400 MHz, CDCl₃) δ=7.49-7.25 (m, 14H), 7.21-7.15 (m, 1H), 6.82 (d, J=8.8 Hz, 4H), 6.46 (s, 1H), 5.65 (d, J=8.2 Hz, 1H), 5.57-5.39 (m, 2H), 4.72 (s, 2H), 4.16-4.07 (m, 2H), 3.93 (dd, J=2.6, 10.8 Hz, 1H), 3.81-3.59 (m, 11H), 3.81-3.59 (m, 1H), 3.24 (dd, J=10.6, 13.5 Hz, 1H), 3.10 (dd, J=9.8, 13.3 Hz, 1H), 2.79 (d, J=2.2 Hz, 1H); ³¹P NMR (CD₃CN) δ=22.37 (s)

### Preparation of 11

To a solution of 10 (4.6 g, 6.06 mmol, 1 eq) and NaI (2.73 g, 18.19 mmol, 3 eq) in MeCN (15 mL) was added chloromethyl 2,2-dimethylpropanoate (3.65 g, 24.25 mmol, 3.51 mL, 4 eq). The mixture was stirred at 85° C. for 24 h. The reaction mixture was concentrated under reduced pressure to give a residue. The residue was purified by flash silica gel chromatography (ISCO®; 40 g SepaFlash® Silica Flash Column, Eluent of 0-50%, EtOAc/PE gradient @ 40 mL/min) to give 11 (2.7 g, 44.6% yield) as a pale yellow solid. LCMS (m/z): 981.1 [M+Na]⁺.

### Preparation of 12

To a solution of 11 (2.7 g, 2.82 mmol, 1 eq) in DCM (20 mL) was added Et₃SiH (645.45 mg, 2.82 mmol, 5 mL, 1 eq), followed by addition of TFA (1.54 g, 13.51 mmol, 1 mL, 4.80 eq). The mixture was stirred at 20° C. for 0.5 h. The reaction mixture was concentrated under reduced pressure to give a residue. The residue was purified by flash silica gel chromatography (ISCO®; 24 g SepaFlash® Silica Flash Column, Eluent of 0-50%, EtOAc/DCM gradient @ 30 mL/min) to give 12 (1.6 g, 84.82% yield) as a pale yellow solid. LCMS (ESI): m/z 679.1 [M+Na]⁺; ¹H NMR (400 MHz, CDCl₃) δ=7.44 (d, J=8.2 Hz, 1H), 7.38-7.26 (m, 5H), 5.76 (d, J=8.2 Hz, 1H), 5.69-5.62 (m, 4H), 5.51-5.43 (m, 1H), 5.51-5.43 (m, 1H), 4.70 (s, 2H), 4.30 (s, 1H), 4.26-4.06 (m, 4H), 3.90 (dd, J=4.9, 8.4 Hz, 2H), 3.22-3.06 (m, 1H), 1.22 (s, 18H): ³¹P NMR (162 MHz, CD₃CN) δ=20.25 (s, 1P).

### Preparation of 13

To a mixture of 12 (1.4 g, 2.13 mmol, 1 eq) in isopropanol (20 ml) and H₂O (2 mL) added Pd/C (1.4 g) and HCOOH (51.22 mg, 1.07 mmol, 2 mL) under N₂. The suspension was degassed under vacuum and purged with H₂ several times. The mixture was stirred under H₂ (15 PSI) at 15° C. for 5 h. The reaction mixture was filtered and the filtrate was concentrated to give a residue. The residue was purified by flash silica gel chromatography (ISCO®; 24 g SepaFlash® Silica Flash Column, Eluent of 0-50%, EtOAc/DCM gradient @ 30 mL/min) to give 13 (848 mg, 74.14% yield) as a white foam. LCMS (ESI): m/z 537.0 [M+H]⁺; ¹H NMR (400 MHz, CDCl₃) δ=10.01 (s, 1H), 7.53 (d, J=8.0 Hz, 1H), 5.78-5.63 (m, 6H), 4.40 (s, 1H), 4.35-4.22 (m, 3H), 4.11 (d, J=1.5 Hz, 1H), 3.88 (d, J=8.5 Hz, 2H), 1.22 (s, 18H); ³¹P NMR (162 MHz, CD₃CN) δ=20.17 (s, 1P.)

### Preparation of 14

To a solution of 13 (848 mg, 1.58 mmol, 1 eq) in DCM (10 mL) was added 3-bis(diisopropylamino)phosphanyloxypropanenitrile (571.73 mg, 1.90 mmol, 602.45 uL, 1.2 eq) at 0° C., followed by addition of 1H-imidazole-4,5-dicarbonitrile (186.7 mg, 1.58 mmol, 1 eq). The mixture was stirred at 15° C. for 1 h. The reaction mixture was quenched by addition of sat. aq. NaHCO₃ (10 mL) and diluted with DCM (20 mL). Then the organic layer was washed with sat. aq. NaHCO₃ (10 mL*2), dried over Na₂SO₄, filtered and concentrated under reduced pressure. The residue was purified by flash silica gel chromatography (ISCO®; 12 g SepaFlash® Silica Flash Column, Eluent of 0-50%, phase A: PE with 0.5% TEA; phase B: EA with 10% EtOH, 30 mL/min) to give 14 (720 mg, 61.21% yield) as a colorless oil. LCMS (ESI): m/z 737.1 [M+H]⁺; ¹H NMR (400 MHz, CD₃CN) δ=9.17 (s, 1H), 7.49 (d, J=8.0 Hz, 1H), 5.91-5.77 (m, 1H), 5.65-5.54 (m, 5H), 4.49-4.26 (m, 2H), 4.23-4.07 (m, 2H), 3.92-3.55 (m, 6H), 2.71-2.61 (m, 2H), 1.24-1.16 (m, 30H): ³¹P NMR (162 MHz, CD₃CN) δ=151.59.

### Example 52: Synthesis of 102

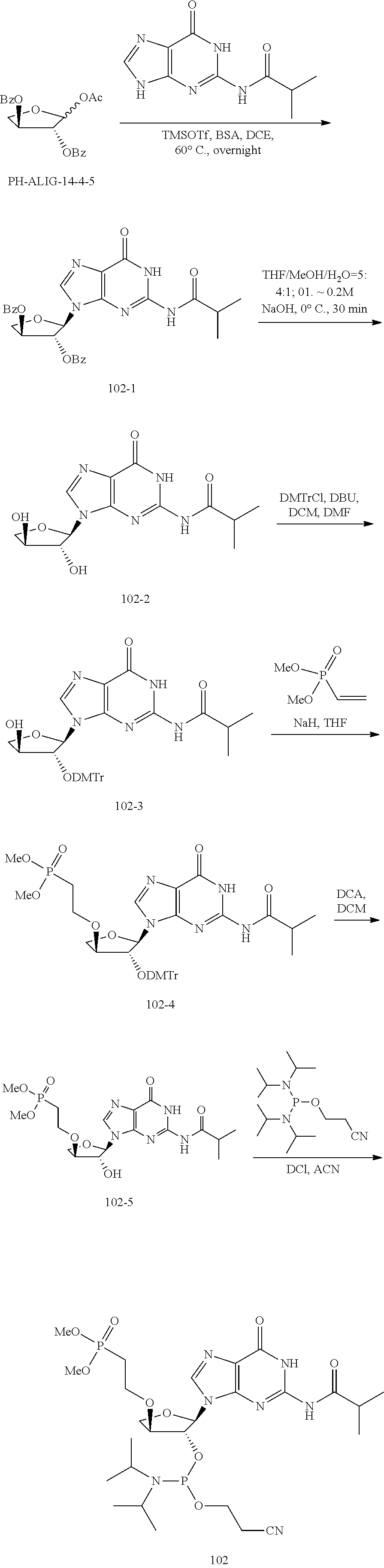

### Example 53: Synthesis of 103

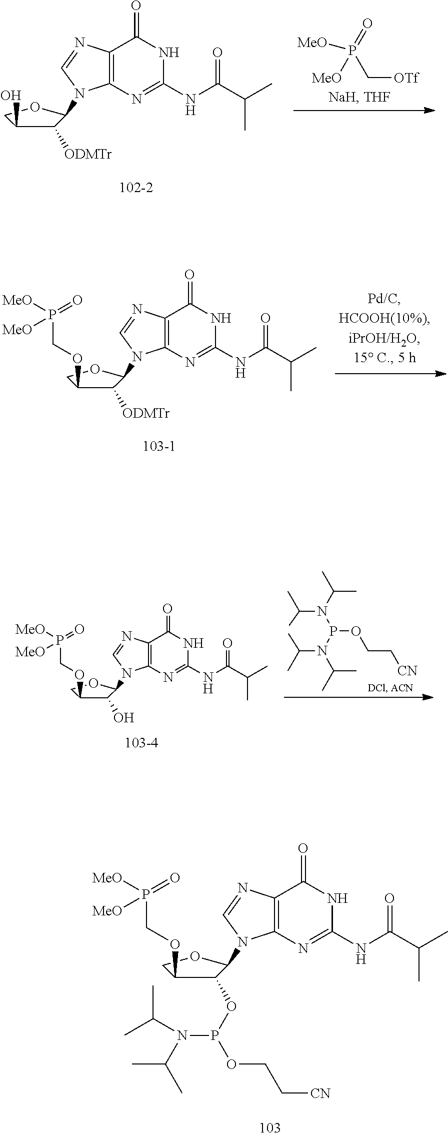

### Example 54: Synthesis of 104

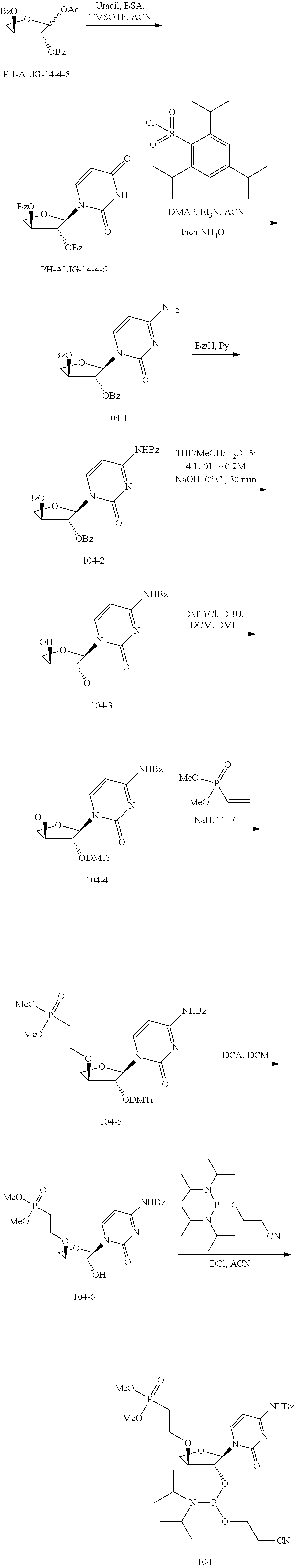

### Example 55: Synthesis of 105

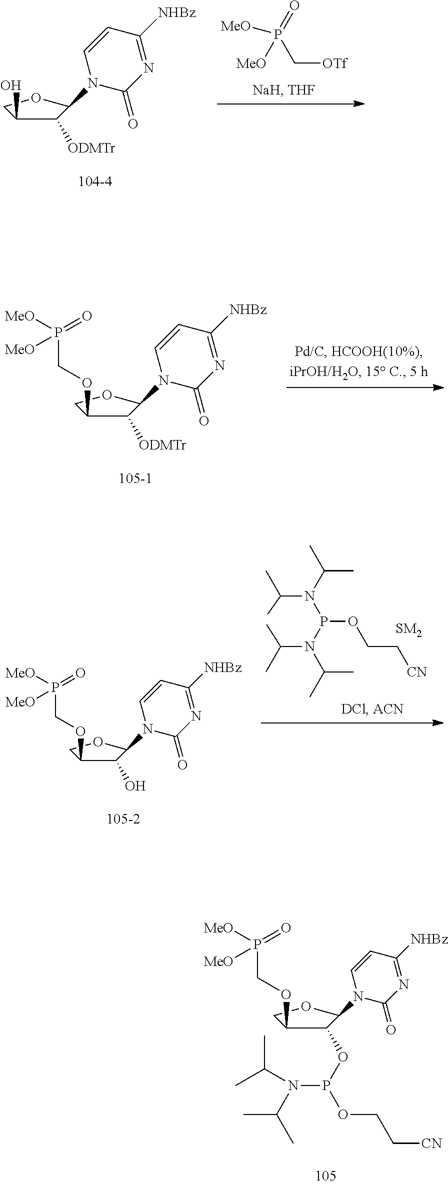

### Example 56

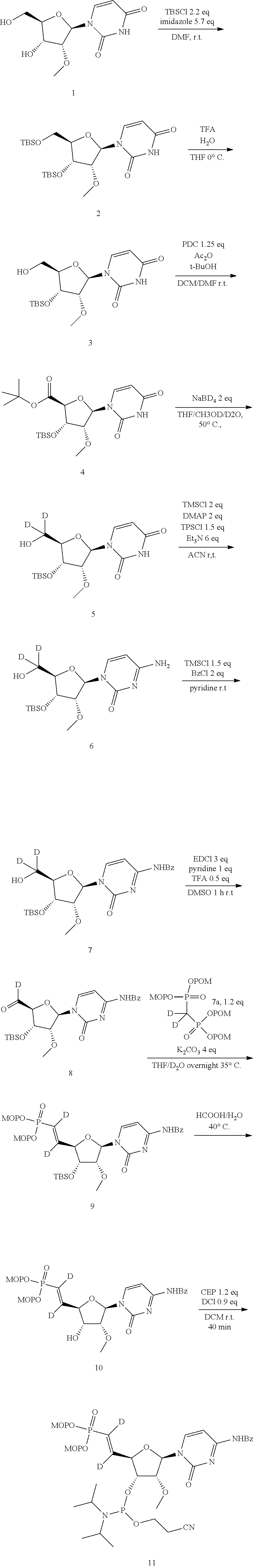

Preparation of 2: A 2 L three-necked round bottom flask equipped with magnetic stirrer and thermometer was charged with 1 (60.0 g, 228.8 mmol) in dry DMF (600.0 mL) at r.t., imidazole (95.2 g, 1.3 mol) was added into the mixture reaction, then the reaction mixture was cooled down to turn 5° C., TBSCl (76.8 g, 499.3 mmol) was added into the mixture reaction, the reaction mixture was allowed to stir for 12 h at r.t. 1 was consumed by LCMS, then the reaction mixture was added in the saturated sodium bicarbonate solution (1.0 L), after quenching the reaction, the aqueous layer was extracted with EA (400.0 mL*2), the combined organic layer was washed with saturated brine and dried over anhydrous sodium sulfate, the organic layer was concentrated to get crude 2 (110.2 g, 212.8 mmol, 93.1% yield) as a white solid, the crude product was used directly for the next step without purification. ESI-LCMS: m/z=487.3 [M+H]⁺.

Preparation of 3: A 3 L three-necked round bottom flask equipped with magnetic stirrer and thermometer was charged with 2 (117.0 g, 225.9 mmol) in THF (550.0 mL) at r.t., water (275.0 mL) was added into the mixture reaction, then the reaction mixture was cooled down to turn 0° C. and add TFA (275.0 mL) by constant pressure funnel after 4 h, the reaction mixture was allowed to stir for 2 h at 0° C. 2 was consumed by TLC. Then, reaction mixture was added in a mixture solvent of ammonium hydroxide (250.0 mL) and water (800.0 mL) at 0° C., after quenching the reaction, the aqueous layer was extracted with EA (500.0 mL*2), the combined organic layer was washed with saturated brine and dried over anhydrous sodium sulfate, the organic layer was concentrated to get crude which was purified by silica gel column chromatography (PE:EA=10:1 to 0:1) to give compound 3 (51.1 g, 59.3% yield) as a white solid. ¹H-NMR (600 MHz, DMSO-d₆): δ=11.35 (s, 1H), 7.919 (d, J=6 Hz, 1H), 5.82 (s, 1H), 5.65 (d, J=6 Hz, 1H), 5.18 (s, 1H), 4.29 (s, 1H), 3.83 (s, 2H), 3.65 (d, J=12 Hz, 1H), 3.53 (d, J=6 Hz, 1H), 3.32 (d, J=6 Hz, 1H), 0.87 (s, 9H), 0.08 (s, 6H). ESI-LCMS: m/z=373.1 [M+H]⁺.

Preparation of 4: A 3 L three-necked round bottom flask equipped with magnetic stirrer and thermometer was charged with 3 (50.0 g, 131.5 mmol) in a mixture solvent of DCM (250.0 mL) and DMF (70.0 mL) at r.t., the mixture solution was cooled down to turn 5° C., PDC (63.1 g, 164.4 mmol) and 1-BuOH (200.0 mL) were added into the mixture reaction, keep the reaction at 5° C. and add Ac₂O (130.0 mL) by constant pressure funnel after 0.5 h, the reaction mixture was allowed to stir for 4 h at r.t. 3 was consumed by lc-ms, then the reaction mixture was added in the saturated sodium bicarbonate (400.0 mL), after quenching the reaction, the aqueous layer was extracted with DCM (500.0 mL*2), the combined organic layer was washed with saturated brine and dried over anhydrous sodium sulfate, the organic layer was concentrated to get crude which was purified by silica gel column chromatography (PE:EA=10:1 to 2:1) to give compound 4 (29.8 g, 50.6% yield) as a white solid. ¹H-NMR (DMSO d₆): δ=11.42 (s, 1H), 8.04 (d, J=6 Hz, 1H), 5.82 (s, 1H), 5.78 (d, J=6 Hz, 1H), 4.44 (s, 1H), 4.25 (s, 1H), 3.84 (s, 1H), 3.32 (s, 3H), 1.46 (s, 9H), 0.89 (s, 9H), 0.12 (s, 6H). ESI-LCMS: m/z=443.1 [M+H]⁺.

Preparation of 5: To a solution of 4 (33.0 g, 74.7 mmol) in dry THF (330.0 mL) was added CH₃OD (66.0 mL) and D₂O (33.0 mL) at r.t. Then the reaction mixture was added NaBD₄ (9.4 g, 224.0 mmol) three times per an hour at 50° C. The solution was stirred at 50° C. for 3 h. LCMS showed 4 was consumed. Water (300.0 mL) was added. The product was extracted with EA (2*300.0 mL). The organic layer was washed with brine and dry over by Na₂SO₄. Then the solution was concentrated under reduced pressure, crude was purified by by silica gel column chromatography (PE:EA=10:1 to 3:1) to give 5 (19.1 g, 68.5% yield) as a white solid. ¹H-NMR (600 MHz, DMSO d₆): δ=11.35 (s, 1H), 7.92-7.91 (d, J=6 Hz, 1H), 5.83-5.82 (d, J=6 Hz, 1H), 5.66-5.65 (d, J=6 Hz, 1H), 5.14 (s, 1H), 4.30-4.28 (m, 1H), 3.84-3.82 (m, 2H), 3.34 (s, 3H), 0.88 (s, 9H), 0.09 (s, 6H). ESI-LCMS: m/z 375 [M+H]⁺.

Preparation of 6: To a solution of 5 (19.1 g, 51.1 mmol) in dry ACN (190.0 mL) was added Et₃N (20.7 g, 204.6 mmol) at r.t. and TMSCl (11.1 g, 102.1 mmol) at 0° C. Then the reaction mixture was stirred at r.t. for 40 min. LCMS showed 5 was consumed and an intermediate was formed. Then the solution was added DMAP (12.5 g, 102.3 mmol), Et₃N (10.3 g, 102.1 mmol) and TPSCl (23.2 g, 76.6 mmol). The reaction mixture was stirred at r.t. for 15 h. LCMS showed the intermediate was consumed and conformed another intermediate. Then was added NH₄OH (200.0 mL) and stirred at r.t. for 24 h to give the mixture of product. The product was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/2 increasing to CH₃CN/H₂O=1/0 within 20 min, the eluted product was collected at CH₃CN/H₂O=1/0; Detector, UV 254 nm. This resulted in to give 6 (14.0 g, 73.7% yield). ¹H-NMR (DMSO-d₆): δ=7.89-7.88 (d, J=6 Hz, 1H), 7.20-7.18 (d, J=12 Hz, 2H), 5.85-5.84 (d, J=6 Hz, 1H), 5.73-5.72 (d, J=6 Hz, 1H), 5.09 (s, 1H), 4.24-4.23 (m, 1H), 3.81-3.80 (d, J=6 Hz, 1H), 3.69-3.68 (m, 1H), 3.36 (s, 3H), 0.87 (s, 9H), 0.07 (s, 6H). ESI-LCMS: m/z 374 [M+H]⁺.

Preparation of 7: To a solution of 6 (14.0 g, 37.5 mmol) in pyridine (140.0 mL) was added TMSCl (6.3 g, 58.0 mmol) at 0° C. and the mixture was stirred at r.t. for 1.5 h. LCMS showed 6 was consumed and an intermediate (a) was formed. Then was added BzCl (10.8 g, 76.8 mmol) at 0° C. and the mixture was stirred at r.t. for 1.5 h. LCMS showed the intermediate was consumed and another intermediate was formed. Then the mixture was added NH₄OH (30.0 mL) and was stirred at r.t. for 15 h. LCMS showed the intermediate was consumed. Water (300.0 mL) was added. The solution was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=1/0; Detector, UV 254 nm. This resulted in to give 7 (10.5 g, 58.6% yield). ¹H-NMR (600 MHz, DMSO d₆): δ=11.29 (s, 1H), 8.53-8.52 (d, J=6 Hz, 1H), 8.01-8.00 (d, J=6 Hz, 2H), 7.63-7.61 (m, 1H), 7.52-7.50 (m, 2H), 7.36 (s, 1H), 5.88 (s, 1H), 5.24 (s, 1H), 4.28-4.26 (m, 1H), 3.91 (s, 1H), 3.81-3.79 (m, 1H), 3.46 (s, 3H), 0.87 (s, 9H), 0.08 (s, 6H). ESI-LCMS: m/z 478 [M+H]⁺.

Preparation of 8: To a solution of 7 (10.5 g, 22.0 mmol) in DMSO (105.0 mL) was added EDCI (12.7 g, 66.0 mmol), dry pyridine (1.7 g, 22.0 mmol) at r.t. and TFA (1.3 g, 11.0 mmol) at 0° C. Then the reaction mixture was stirred for 1 h. LCMS showed 7 was consumed. Water (100.0 mL) was added. The solution was extracted with EA (2*200.0 mL). The organic layer was washed with brine and dry over by Na₂SO₄. Then the solution was concentrated under reduced pressure to give the crude product 8 which was used in next step directly. ESI-LCMS: m/z 475 [M+H]⁺.

Preparation of 9: To a solution of 8 in dry THF (120.0 mL) and D₂O (40.0 mL) was added K₂CO₃ (12.2 g, 88.1 mmol) and 7a (16.8 g, 26.5 mmol) then the reaction mixture was stirred for 15 h at 35° C. under the N₂ atmosphere. LCMS showed 95% 7 was consumed. Water (60.0 mL) was added. The solution was extracted with EA (2*150.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=4/1; Detector, UV 254 nm. This resulted in to give 9 (9.3 g, 54.1% yield). ¹H-NMR (DMSO-d₆) δ=11.33 (s, 1H), 8.17-8.15 (d, J=12, 1H), 8.02-8.00 (d, J=12, 1H), 7.64-7.62 (m, 1H), 7.53-7.50 (m, 2H), 7.44-7.42 (d, J=12, 1H), 4.46-4.44 (d, J=12, 1H), 4.24-4.23 (d, J=6, 1H), 3.93-3.91 (d, J=12, 1H), 1.16 (s, 18H), 0.86 (s, 9H)), 0.08-0.06 (d, J=12, 6H). ESI-LCMS: m/z 782 [M+H]⁺; ³¹P-NMR (DMSO-d₆) δ=16.77, 16.00.

Preparation of 10:9 (9.3 g, 11.9 mmol) in the mixture solution of HOAc (140.0 mL) and H₂O (140.0 mL) was stirred at 30° C. for 15 h. LCMS showed 9 was consumed. The solution was added in the ice water and extracted with EA (2*300.0 mL). The organic layer was quenched to pH=6-7 and then washed with brine and dry over Na₂SO₄. Then the solution was concentrated under reduced, crude was purified by pressure Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=2.5/1; Detector, UV 254 nm. This resulted in to give 10 (5.1 g, 64.6% yield). ¹H-NMR (DMSO-d₆) δ=9.09 (s, 1H), 7.92-7.85 (m, 3H), 7.60-7.48 (m, 4H), 6.02 (s, 1H), 5.71-5.64 (m, 4H), 4.53-4.51 (m, 1H), 3.94-3.70 (m, 5H), 3.31 (s, 1H), 1.21 (s, 18H). ³¹P-NMR (DMSO-d₆) δ=16.45. ESI-LCMS: m/z 668 [M+H]⁺.

Preparation of 11: To a suspension of 10 (4.6 g, 6.9 mmol) in DCM (45.0 mL) added CEOP[N(ipr)₂]₂ (2.5 g, 8.3 mmol), DCI (730.4 mg, 6.2 mmol). The mixture was stirred at r.t. for 1 h. LCMS showed 10 was consumed completely. The solution was quenched by water (40.0 mL), washed with brine (2*20.0 mL) and dry over by Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O=1/1 increasing to CH₃CN/H₂O=1/0 within 20 min, the eluted product was collected at CH₃CN/H₂O=4/1; Detector, UV 254 nm. This resulted in to give 11 (4.7 g, 5.4 mmol, 78.3% yield) as a white solid. ¹H-NMR (600 MHz, DMSO-d₆) δ=11.34 (s, 1H), 8.18-8.16 (m, 1H), 8.02-8.01 (d, J=6, 2H), 7.65-7.42 (m, 4H), 5.95-5.93 (m, 1H), 5.66-5.61 (m, 4H), 4.64-4.57 (m, 1H), 4.32-4.31 (d, J=6, 1H), 4.12-4.10 (m, 1H), 3.81-3.45 (m, 7H), 2.81-2.79 (m, 2H), 1.16-1.13 (m, 30H). ³¹P-NMR (CDCl₃-d₆) δ=150.65, 150.20, 16.64, 15.41. ESI-LCMS: m/z 868 [M+H]⁺.

### Example 57

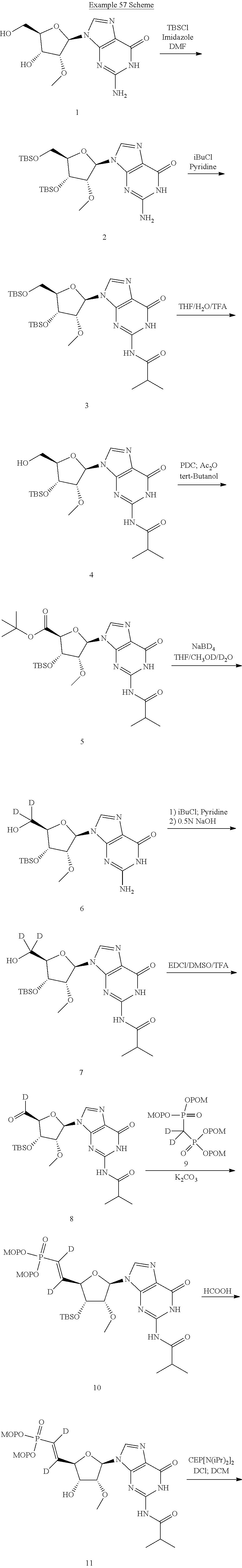

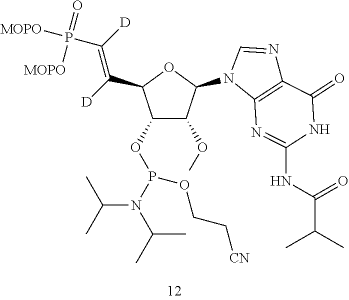

Preparation of 2: 1 (94.5 g, 317.9 mmol) was dissolved in dry DMF (1000 mL) under N₂ atmosphere. To the solution TBSCl (119.3 g, 794.7 mmol) and imidazole (75.8 g, 1.1 mol) was added at 25° C. and stirred for 17 hr. LCMS showed all of 1 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 2 (200 g, crude). ESI-LCMS: m/z 526 [M+H]⁺.

Preparation of 3: 2 (175.1 g, 333.0 mmol) was evaporated with pyridine and dried in vacuo for two times. The residue was dissolved in pyridine (1500 mL) under N₂. To the solution, i-BuCl (88.7 g, 832.6 mmol) was added at 5° C. under N₂ atmosphere and stirred for 3 hr. LCMS showed all of 2 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 3 (228 g, crude). ESI-LCMS: m/z 596 [M+H]⁺.

Preparation of 4: A solution of 3 (225 g, 377.6 mmol) was in THF (2000 mL) was added H₂O (500 mL) and TFA (500 mL) was added at 5° C. Then the reaction mixture was stirred at 5° C. for 1 hr. LCMS showed all of 3 consumed. Con NH₄OH (aq) was added to mixture to quench the reaction until the pH=7-8, then washed with H₂O (2000*2 mL), EA (2000*2 mL) and brine (1500 mL). Dried over Na₂SO₄ and concentrated to give crude which was purified by cc. The reaction mixture was concentrated to give 4 (155.6 g, 83.9% yield). ESI-LCMS: m/z 482 [M+H]⁺.

Preparation of 5: 4 (100 g, 207.6 mmol) was dissolved in dry DMF (1000 mL) under N₂. To the solution, t-BuOH (307.8 g, 4.2 mol), PDC (156.1 g, 0.4 mol) and Ac₂O (212.0 g, 2.1 mol) was added at 25° C. under N₂ atmosphere and stirred at 25° C. for 2 hr. LCMS and TLC showed all of 4 consumed. NaHCO₃(aq) was added to mixture to quench the reaction until the pH=7-8, then washed with H₂O (500*2 mL), EA (500*2 mL) and brine (500 mL). Dried over Na₂SO₄ and concentrated to give crude which was purified by cc. and MPLC. The reaction mixture was concentrated to give 5 (77.3 g, 61.6% yield). ESI-LCMS: m/z 552 [M−H]⁺.

Preparation of 6: 5 (40.0 g, 72.6 mmol) was dissolved in dry THF (400 mL) under N₂. To the solution, MeOD (80 mL) and D₂O (40 mL) was added at 25° C. under N₂ atmosphere, then NaBD₄ (9.1 g, 217.4 mmol) was added for three times and stirred for 15 hr. LCMS and TLC showed all of 5 consumed. The mixture was concentrated to give crude which goes to the next step. The reaction mixture was concentrated to give crude 6 (30 g, crude). ESI-LCMS: m/z 414 [M+H]⁺

Preparation of 7: 6 (30 g, crude) was evaporated with pyridine and dried in vacuo for two times. The residue was dissolved in dry pyridine (300 mL) under N₂. Then iBuCl (15.5 g, 145.3 mmol) was slowly added to the reaction mixture at 0° C. under N₂ atmosphere and stirred at 25° C. for 1 hr. LCMS and TLC showed all of 6 consumed. NaHCO₃(aq) was added to mixture to quench the reaction until the pH=7.5, then washed with H₂O (1500 mL), EA (1000*2 mL) and brine (1500 mL). Dried over Na₂SO₄ and concentrated to give crude residue R¹. NaOH (8 g, 0.2 mol), MeOH (80 mL) and H₂O (20 mL) made up NaOH (aq). The residue R¹ (40 g, 3.63 mmol) was dissolved in pyridine (20 mL). To the solution, 2N NaOH (aq) (100 ml) was added to the solution and stirred the reaction 15 min at 5° C. TLC showed all of R¹ consumed. The mixture was added NH₄Cl to pH=7-8 at 5° C., and concentrated to give crude which was purified by cc. The product was concentrated to give 7 (15.5 g, 33.00% yield over two steps). ESI-LCMS: m/z 484 [M+H]⁺.

Preparation of 8: To a stirred solution of 7 (15.5 g, 32.1 mmol) in DMSO (150 mL) were added EDCI (18.5 g, 96.3 mmol), pyridine (2.5 g, 32.1 mmol), TFA (1.8 g, 16.0 mmol) at room temperature under N₂ atmosphere. The reaction mixture was stirred for 1 h at room temperature. The reaction was quenched with water, extracted with EA (300.0 mL), washed with brine, dried over Na₂SO₄ and evaporated under reduced pressure give a crude 8 (17.3 g, crude) which was used directly to next step. ESI-LCMS: m/z=481 [M+H]⁺.

Preparation of 10: A solution of 8 (17.3 g, crude), 9 (21.4 g, 33.7 mmol) and K₂CO₃ (13.3 g, 96.3 mmol) in dry THF (204 mL) and D₂O (34 mL) was stirred 5 h at 40° C. The mixture was quenched with water, extracted with EA (600.0 mL), washed with brine, dried over Na₂SO₄ and evaporated under reduced pressure. The residue was purified by silica gel (PE:EA=5:1 ˜1:1) to give 10 (9.3 g, 36.6% yield over 2 steps) as a white solid. ESI-LCMS m/z=787 [M+H]⁺.

¹H-NMR (DMSO-d₆): δ 11.24 (s, 1H, exchanged with D₂O), 8.74 (d, J=2.7 Hz, 2H), 8.05-8.04 (d, J=7.4 Hz, 2H), 7.65 (t, 1H), 7.57-7.54 (t, 2H), 6.20 (d, J=5.0 Hz, 1H), 5.64-5.58 (m, 4H), 4.77 (t, 1H), 4.70 (t, 1H), 4.57-4.56 (t, 1H), 3.35 (s, 3H), 1.09 (d, J=6.5 Hz, 18H), 0.93 (s, 9H), 0.15 (d, J=1.8 Hz, 6H); ³¹P NMR (DMSO-d₆): δ 17.05.

Preparation of 11: To a round-bottom flask was added 10 (9.3 g, 11.5 mmol) in a mixture of H₂O (93 mL) and HCOOH (93 mL). The reaction mixture was stirred for 5 h at 50° C. and 15 h at 35° C. The mixture was extracted with EA (500.0 mL), washed with water, NaHCO₃ solution and brine successively, dried over Na₂SO₄ and evaporated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 20 min, the eluted product was collected at CH₃CN/H₂O (0.5% NH₄HCO₃)=3/2; Detector, UV 254 nm. To give product 11 (6.3 g, 78% yield). ¹H-NMR (600 MHz, DMSO-d₆): δ 12.17 (s, 1H, exchanged with D₂O), 11.51 (s, 1H), 8.28 (s, 1H), 6.02-6.03 (d, J=4.2 Hz, 1H), 5.63-5.72 (m, 5H), 4.60 (s, 1H), 4.43-4.45 (m, 2H), 3.40 (s, 1H), 3.38 (s, 1H), 2.83-2.88 (m, 1H), 1.15-1.23 (m, 24H); ³¹P NMR (DMSO-d₆) δ=17.69. ESI-LCMS m/z=674 [M+H]⁺.

Preparation of 12: To a solution of 11 (5.6 g, 8.3 mmol) in DCM (55.0 mL) was added the DCI (835 mg, 7.1 mmol), then CEP[N(ipr)₂]₂ (3.3 g, 10.8 mmol) was added. The mixture was stirred at r.t. for 1 h. The reaction mixture was washed with H₂O (50.0 mL) and brine (50.0 mL), dried over Na₂SO₄ and evaporated under pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=9/1; Detector, UV 254 nm. The product was concentrated to give 12 (6.3 g, 87% yield) as a white solid. ¹H-NMR (DMSO-d₆): δ 12.14 (s, 1H, exchanged with D₂O), 11.38 (s, 1H), 8.27-8.28 (d, J=6 Hz, 1H), 5.92-5.98 (m, 1H), 5.59-5.65 (m, 4H), 4.57-4.68 (m, 3H), 3.61-3.85 (m, 4H), 3.37 (s, 1H), 3.32 (s, 1H), 2.81-2.85 (m, 3H), 1.09-1.20 (m, 36H); ³¹P NMR (DMSO-d₆): δ 150.60, 149.97, 17.59, 17.16; ESI-LCMS m/z=874 [M+H]⁺.

### Example 58. Luciferase Reporter Assay in COS-7 Cells

All siNAs synthesized were tested for in vitro activity using a 3-point luciferase reporter assay and a subset of candidates were tested in a dose-response luciferase reporter assay.

In the psiCHECK™-2 reporter plasmid, Renilla luciferase is used as the primary reporter gene with the HSD17B13 gene (NM_178135.5) cloned downstream of its translational stop codon. A second reporter gene, firefly luciferase, is also expressed and used as a transfection control. COS-7 cells (ATCC, CRL-1651) were seeded into 96-well microplates and transfected with the reporter plasmid using Lipofectamine 3000 (Invitrogen, L3000001). The cells were then transfected with 10, 1, or 0.1 nM siNAs using Lipofectamine RNAiMAX

(Invitrogen, 13778100). A mock, no-drug control, which consisted of transfecting 1× phosphate-buffered saline, was included. After 72 hours of siNA treatment, the Dual-Glo® Luciferase Assay System (Promega, E2940) was used according to the manufacturer's protocol to quantify firefly and Renilla luciferase activity. All luminescence was measured on an En Vision plate reader (Perkin Elmer). The Renilla: firefly luminescence ratio was calculated for each well. The ratios from siNA-treated wells were then normalized to ratios of the mock-treated wells and percentage inhibition was calculated. CellTiter-Glo® Luminescent Cell Viability Assays were run in parallel using similarly treated COS-7 cells. Assays were performed according to the manufacturer's protocol and luminescence was measured on an En Vision plate reader. The luminescence from siNA-treated wells were then normalized to luminescence of mock-treated wells and percentage viability was calculated.

A subset of siNA candidates were then tested in a dose-response luciferase reporter assay. Dose-response assays were similarly conducted, but instead with serial concentrations of siNAs starting at 10 nM (1:5 dilutions) for a total of nine concentrations tested for each siNA. Dose-response curves were fitted by nonlinear regression with variable slope and EC₅₀ values and maximum percentage inhibition were calculated. No siNAs exhibited significant cytotoxic effects in the COS-7 cells at the concentrations tested.

TABLE 1
Luciferase reporter assay in COS-7 - 3-point data

| ds-siNA | Luciferase reporter assay in COS-7 | | |
|---|---|---|---|
| NO. | % inhibition of reporter activity | | |
| (D#) | 50 nM | 5 nM | 0.5 nM |
| 1 | C | C | A |
| 2 | B | C | C |
| 3 | C | C | D |
| 4 | B | C | C |
| 5 | A | A | B |
| 6 | A | A | B |
| 7 | A | A | A |
| 8 | A | A | B |
| 9 | B | C | C |
| 10 | B | B | C |
| 11 | B | C | C |
| 12 | B | C | C |
| 13 | A | B | C |
| 14 | A | B | C |
| 15 | A | A | A |
| 16 | B | B | C |
| 17 | A | A | B |
| 18 | B | B | C |
| 19 | B | C | C |
| 20 | B | B | C |
| 21 | B | B | C |
| 22 | A | B | C |
| 23 | A | A | B |
| 24 | A | A | B |
| 25 | A | B | B |
| 26 | A | B | C |
| 27 | B | C | D |
| 28 | B | C | D |
| 29 | C | D | D |
| 30 | A | B | C |
| 31 | C | D | D |
| 32 | B | C | D |
| 33 | A | B | B |
| 34 | B | B | C |
| 35 | A | B | C |
| 36 | A | B | B |
| 37 | B | B | C |
| 38 | B | C | D |
| 39 | B | B | C |
| 40 | B | C | C |
| 41 | C | D | D |
| 42 | B | D | D |
| 43 | B | C | D |
| 44 | C | D | D |
| 45 | B | B | D |
| 46 | B | C | D |
| 47 | C | D | D |
| 48 | B | C | D |
| 49 | B | B | D |
| 50 | A | A | B |
| 51 | A | A | B |
| 52 | D | D | D |
| 53 | A | B | C |
| 54 | A | B | C |
| 55 | B | C | D |
| 56 | A | A | B |
| 57 | A | B | C |
| 58 | B | C | C |
| 59 | B | B | C |
| 60 | B | B | C |
| 61 | A | A | B |
| 62 | A | A | C |
| 63 | A | A | B |
| 64 | A | A | C |
| 65 | A | B | C |
| 66 | A | B | C |
| 67 | A | A | B |
| 68 | A | A | C |
| 69 | B | B | C |
| 70 | A | B | C |
| 71 | A | A | B |
| 72 | B | B | C |
| 73 | B | B | C |
| 74 | A | A | B |
| 75 | A | B | C |
| 76 | A | A | B |
| 77 | A | B | B |
| 78 | A | B | B |
| 79 | B | C | D |
| 80 | A | B | C |
| 81 | C | C | D |
| 82 | A | B | C |
| 83 | A | A | B |
| 84 | A | B | B |
| 85 | B | B | D |
| 86 | B | B | D |
| 87 | A | A | B |
| 88 | A | A | B |
| 89 | A | A | B |
| 90 | B | C | D |
| 91 | C | C | D |
| 92 | B | C | D |
| 93 | A | B | B |
| 94 | A | B | B |
| 95 | A | A | B |
| 96 | A | A | B |
| 97 | A | B | B |
| 98 | A | B | B |
| 99 | A | A | B |
| 100 | A | B | C |
| 101 | A | B | D |
| 102 | A | A | B |
| 103 | A | A | B |
| 104 | B | C | D |
| 105 | A | B | C |
| 106 | A | B | C |
| 107 | A | B | C |
| 108 | A | C | D |
| 109 | B | B | C |
| 110 | A | B | C |
| 111 | A | B | C |
| 112 | B | B | C |
| 113 | A | A | B |
| 114 | A | B | C |
| 115 | A | B | B |
| 116 | B | B | D |
| 117 | A | B | C |
| 118 | B | B | C |
| 119 | B | C | D |
| 120 | B | B | C |
| 121 | A | B | B |
| 122 | A | B | C |
| 123 | A | C | C |
| 124 | A | B | C |
| 125 | A | B | C |
| 126 | B | B | D |

A = ≥76%;
B = 51-75%;
C = 26-50%;
D = ≤25%

TABLE 2
CellTiter-Glo in COS-7 - 3-point data

| ds-siNA | CellTiter-Glo in COS-7 | | |
|---|---|---|---|
| NO. | % viability | | |
| (D#) | 50 nM | 5 nM | 0.5 nM |
| 1 | A | A | B |
| 2 | A | A | B |
| 3 | B | A | A |
| 4 | A | A | A |
| 5 | B | A | A |
| 6 | A | A | A |
| 7 | A | A | A |
| 8 | B | A | A |
| 9 | A | A | A |
| 10 | A | A | B |
| 11 | A | A | A |
| 12 | A | A | A |
| 13 | A | A | A |
| 14 | A | A | A |
| 15 | A | A | A |
| 16 | A | A | A |
| 17 | B | A | B |
| 18 | B | A | A |
| 19 | A | A | A |
| 20 | A | A | A |
| 21 | A | A | A |
| 22 | A | A | A |
| 23 | A | A | A |
| 24 | A | A | A |
| 25 | A | A | A |
| 26 | A | A | A |
| 27 | A | A | A |
| 28 | A | A | A |
| 29 | A | A | A |
| 30 | A | A | A |
| 31 | A | A | A |
| 32 | A | A | A |
| 33 | V | A | B |
| 34 | B | A | A |
| 35 | A | A | A |
| 36 | A | A | B |
| 37 | B | A | A |
| 38 | A | A | B |
| 39 | A | A | A |
| 40 | A | A | B |
| 41 | B | A | B |
| 42 | A | A | B |
| 43 | A | B | B |
| 44 | B | B | B |
| 45 | B | B | B |
| 46 | B | B | B |
| 47 | B | B | B |
| 48 | C | B | B |
| 49 | C | A | B |
| 50 | B | B | A |
| 51 | C | B | A |
| 52 | B | A | A |
| 53 | A | B | A |
| 54 | A | A | A |
| 55 | B | B | A |
| 56 | B | B | A |
| 57 | B | B | A |
| 58 | C | B | A |
| 59 | C | A | A |
| 60 | C | A | A |
| 61 | B | A | A |
| 62 | B | B | A |
| 63 | B | B | A |
| 64 | A | A | A |
| 65 | B | A | A |
| 66 | B | A | A |
| 67 | B | B | B |
| 68 | A | A | A |
| 69 | C | B | A |
| 70 | B | B | A |
| 71 | A | A | A |
| 72 | A | A | A |
| 73 | B | A | A |
| 74 | A | A | A |
| 75 | A | A | A |
| 76 | A | A | A |
| 77 | B | A | A |
| 78 | B | A | A |
| 79 | B | A | A |
| 80 | D | B | A |
| 81 | D | B | A |
| 82 | B | A | A |
| 83 | A | A | A |
| 84 | B | A | A |
| 85 | B | A | C |
| 86 | C | A | B |
| 87 | C | A | B |
| 88 | B | A | B |
| 89 | B | A | B |
| 90 | B | A | A |
| 91 | B | A | A |
| 92 | A | A | A |
| 93 | A | A | A |
| 94 | C | B | B |
| 95 | A | A | A |
| 96 | B | B | B |
| 97 | C | A | A |
| 98 | B | A | A |
| 99 | A | A | A |
| 100 | B | A | A |
| 101 | A | A | A |
| 102 | B | A | A |
| 103 | C | A | A |
| 104 | B | A | A |
| 105 | A | A | A |
| 106 | A | A | A |
| 107 | A | A | A |
| 108 | A | A | A |
| 109 | B | A | A |
| 110 | A | A | A |
| 111 | A | A | A |
| 112 | B | A | A |
| 113 | A | A | A |
| 114 | A | A | A |
| 115 | A | A | A |
| 116 | A | A | A |
| 117 | A | A | A |
| 118 | A | A | A |
| 119 | A | A | A |
| 120 | C | A | A |
| 121 | A | A | A |
| 122 | A | A | A |
| 123 | A | A | A |
| 124 | A | A | A |
| 125 | A | A | A |
| 126 | A | A | A |

A = ≥95%;
B = 85-94%;
C = 75-84%;
D = ≤74%

TABLE 3
Luciferase reporter assay in COS-7 - dose-response data

| | Luciferase reporter assay in COS-7 | | |
|---|---|---|---|
| | | Maximum % | CellTiter-Glo in |
| ds-siNA | | inhibition | COS-7 |
| NO. | EC₅₀ | of reporter | CC₅₀ |
| (D#) | (nM) | activity | (nM) |
| 5 | D | BB | >10 |
| 6 | D | BB | >10 |
| 7 | D | AA | >10 |
| 8 | D | CC | >10 |
| 14 | C | AA | >10 |
| 15 | D | BB | >10 |
| 17 | B | AA | >10 |
| 22 | A | CC | >10 |
| 23 | A | BB | >10 |
| 24 | C | BB | >10 |
| 25 | A | BB | >10 |
| 26 | C | BB | >10 |
| 33 | A | AA | >10 |
| 36 | B | BB | >10 |
| 50 | D | BB | >10 |
| 51 | D | BB | >10 |
| 56 | D | BB | >10 |
| 61 | C | BB | >10 |
| 62 | C | BB | >10 |
| 63 | D | BB | >10 |
| 64 | A | AA | >10 |
| 67 | D | BE | >10 |
| 68 | C | AA | >10 |
| 71 | C | CC | >10 |
| 74 | C | AA | >10 |
| 76 | C | BB | >10 |
| 77 | C | AA | >10 |
| 78 | C | BB | >10 |
| 83 | D | AA | >10 |
| 84 | D | BB | >10 |

A = ≥0.51;
B = 0.31-0.5;
C = 0.11-0.3;
D = ≤0.1
AA = ≥91%;
BB = 81-90%;
CC = 71-80%

### Example 59. siNA In Vivo Activity in AAV-HSD17813 Mouse Model

Methods. Certain siRNA molecules were selected for initial pharmacokinetic/pharmacodynamic studies in vivo. To enhance targeted delivery to hepatocytes, a GalNAc ligand was incorporated at the 3′ end of the sense strand via standard phosphoramidite chemistry. The specific GalNAc ligand used (“GalNAc4-ps-GalNAc4-ps GalNAc4” or “p-(ps)2-GalNAc4”), shown below, includes three monomeric “GalNAc4” derivative units linked through two phosphorothioate linkages, where one GalNAc4 unit is linked to the 3′ end nucleotide on the sense strand via a standard phosphodiester linkage.

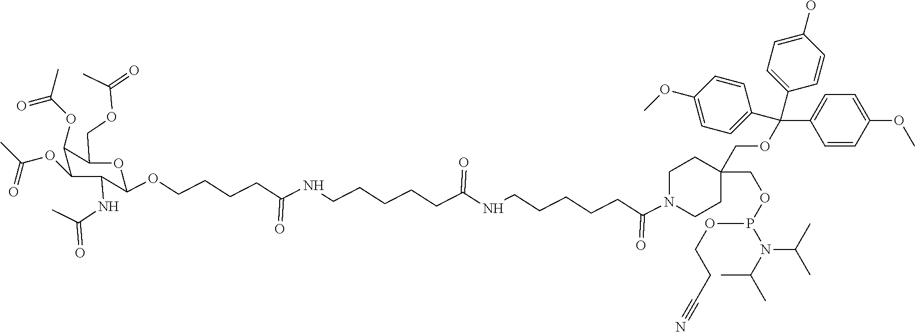 [SMILES: COc1ccc(C(OCC2(COP(OCCC#N)N(C(C)C)C(C)C)CCN(C(=O)CCCCCNC(=O)CCCCCNC(=O)CCCCOC3OC(COC(C)=O)C(OC(C)=O)C(OC(C)=O)C3NC(C)=O)CC2)(c2ccc(O)cc2)c2ccc(OC)cc2)cc1]

### Structure of “GalNAc4 Phosphoramidite”

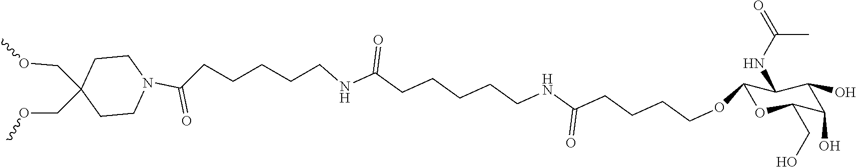

### Structure of “GalNAc4”

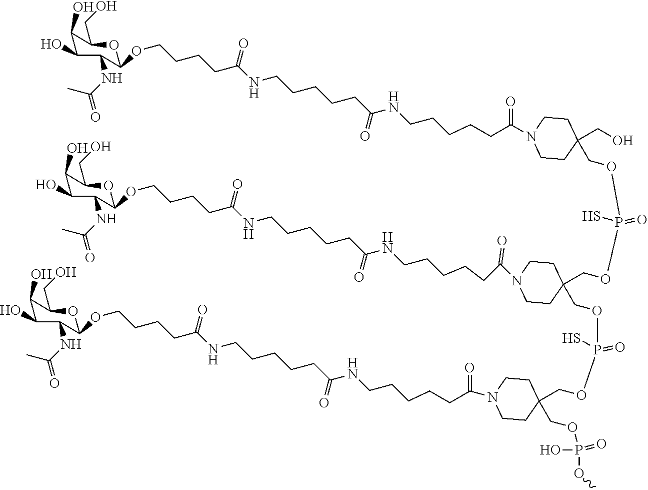

### Structure of “GalNAc4-ps-GalNAc4-ps GalNAc4” or “p-(ps)2-GalNAc4”

To evaluate certain HSD17B13 siRNA agents, an AAV-HSD17ß13 mouse model was used. Mice (n=4/group) were injected with an adeno-associated virus (AAV) expressing human HSD17ß13. Seven days after AAV injection, mice were subcutaneously administered a single dose (ranging from 1.5 to 5 mg/kg) of an siRNA. Seven to fourteen days after administration, human HSD17β13 expression in liver was determined by RTqPCR and western blot analysis after normalization against human HSD17β13 expression of control animals injected with PBS.

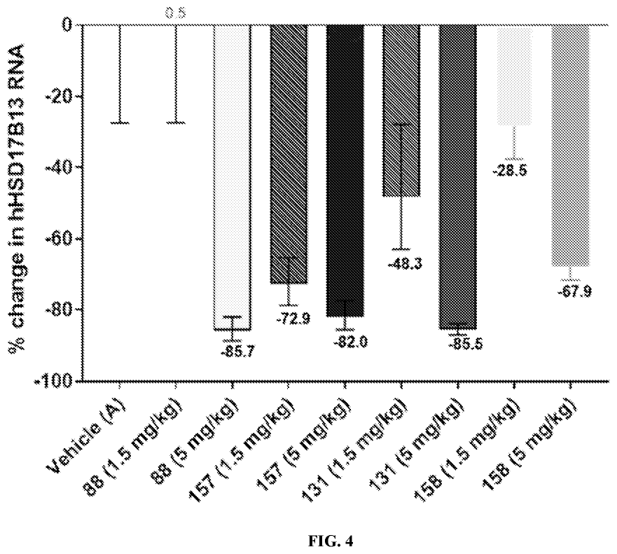

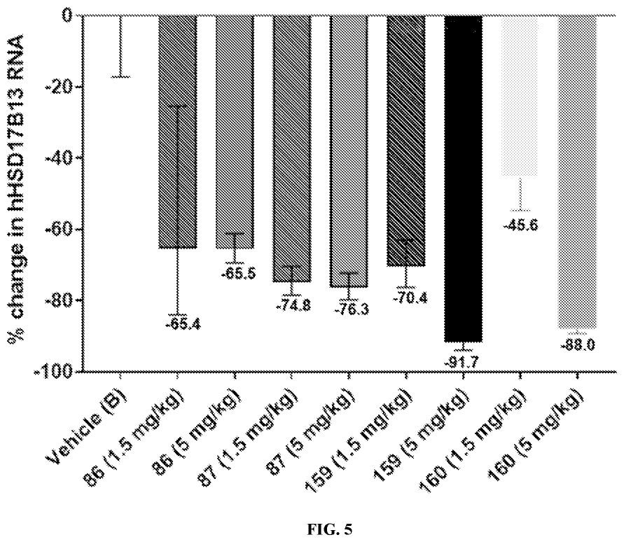

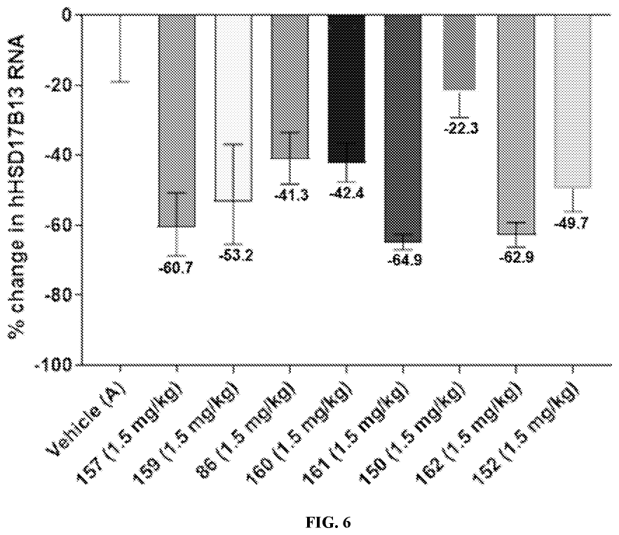

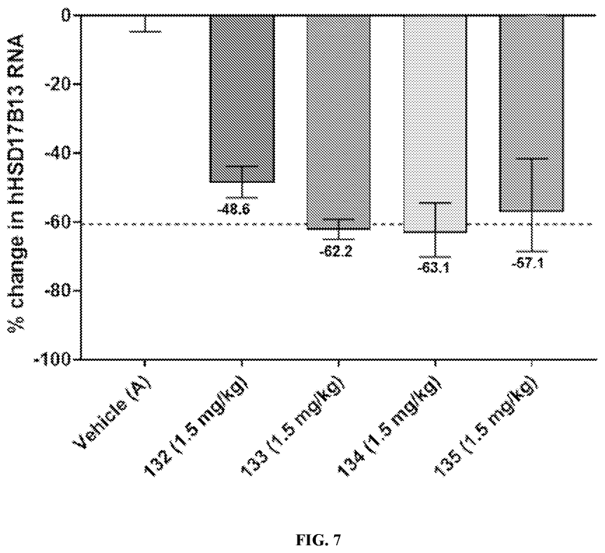

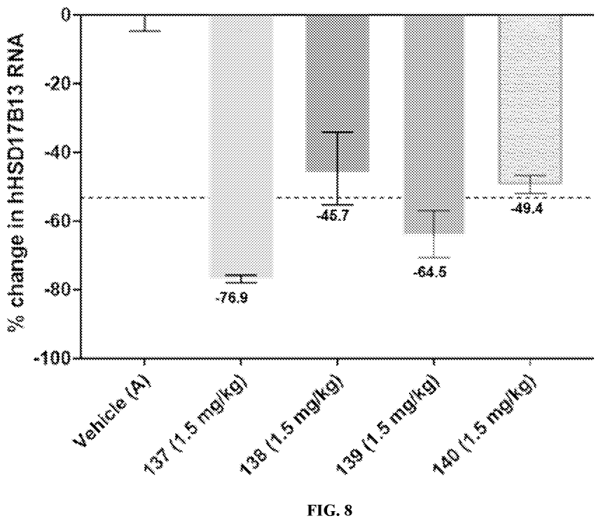

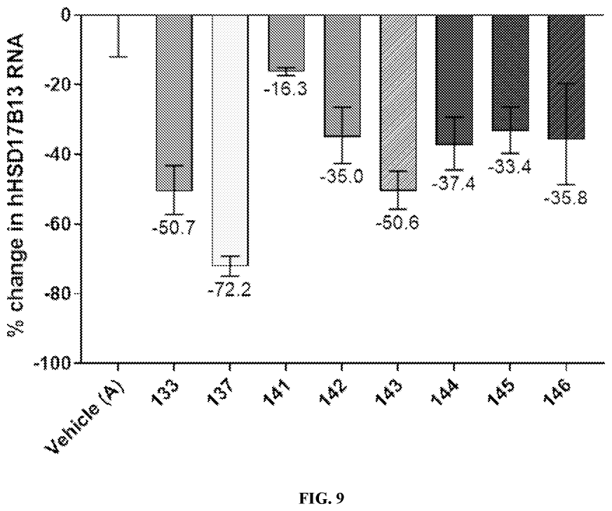

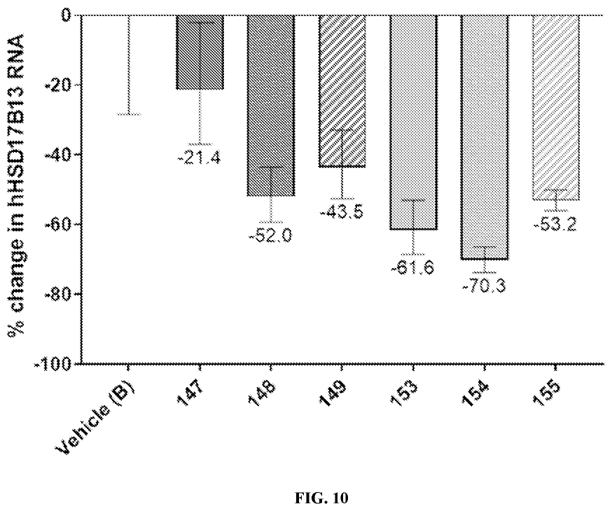

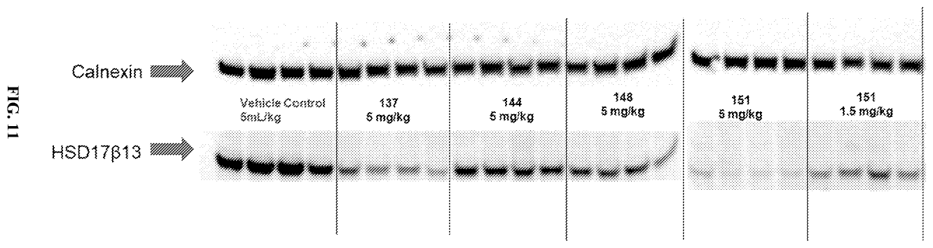

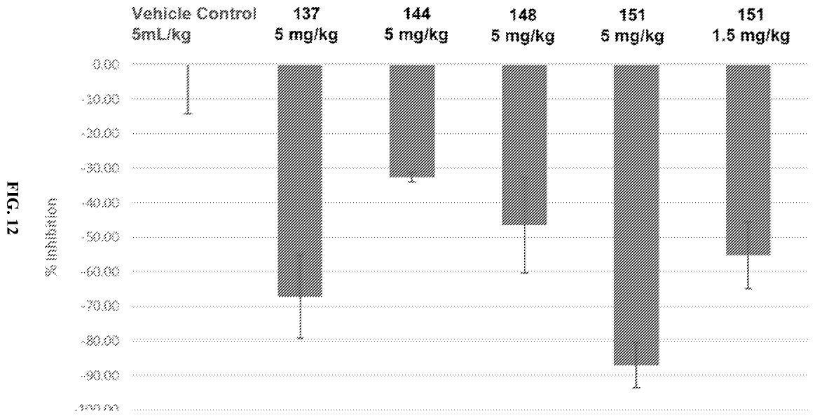

In a first study, a selection of siRNA duplexes were tested at 1.5 and/or 5 mpk, observing the HSD17B13 mRNA knockdown 7 days post dose. The results are shown in FIGS. 4-10 and 13 and Table 4.

TABLE 4
In Vivo Study

| | | | | | %RNA Inhibition | |
|---|---|---|---|---|---|---|
| mds- | SEQ | Sense Strand Base | | Antisense Strand Base | (Drug v. Vehicle) | |
| siNA | ID | Sequence + Modifications | SEQ ID | Sequence + Modifications | 1.5 | 5 |
| (MD#) | NO. | (5′-3′) | NO. | (5′-3′) | mg/kg | mg/kg |
| 88 | 403 | mUpsmUpsmGmGmGmAfAmA | 533 | vmUpsfUpsmUmGmAfU | 0 | 85.7 |
| | | fAfAfCmUmGmGmUmAmUm | | mAmCmCmAmGmUmUf | | |
| | | CmAmApsmA- | | UmUfUmCmCmCmAmA | | |
| | | GalNAc4psGalNAc4psGalNAc4 | | psmUpsmU | | |
| 157 | 576 | mCpsmUpsmUmGmGmAfGmU | 450 | vmApsfApsmCmUmUfCm | 66.8* | 82 |
| | | fCfGfUmUmGmGmUmGmAm | | AmCmCmAmAmCmGfA | | |
| | | AmGmUpsmU- | | mCfUmCmCmAmAmGps | | |
| | | GalNAc4psGalNAc4psGalNAc4 | | mUpsmU | | |
| 131 | 576 | mCpsmUpsmUmGmGmAfGmU | 604 | mApsfApsmCmUmUfCm | 48.3 | 85.5 |
| | | fCfGfUmUmGmGmUmGmAm | | AmCmCmAmAmCmGfA | | |
| | | AmGmUpsmU- | | mCfUmCmCmAmAmGps | | |
| | | GalNAc4psGalNAc4psGalNAc4 | | mUpsmU | | |
| 158 | 601 | mUpsmGpsmAmAmGmUfCmG | 501 | vmApsfUpsmCmAmGfA | 28.5 | 67.9 |
| | | fUfAfAmGmAmAmGmUmCm | | mCmUmUmCmUmUmAf | | |
| | | UmGmApsmU- | | CmGfAmCmUmUmCmA | | |
| | | GalNAc4psGalNAc4psGalNAc4 | | psmUpsmU | | |
| 86 | 401 | mUpsmApsmUmUmGmGfUmU | 531 | vmUpsfApsmAmUmAfU | 53.3* | 65.5 |
| | | fCfUfGmUmGmGmGmAmUm | | mCmCmCmAmCmAmGf | | |
| | | AmUmUpsmA- | | AmAfCmCmAmAmUmA | | |
| | | GalNAc4psGalNAc4psGalNAc4 | | psmUpsmU | | |
| 87 | 402 | mCpsmUpsmAmGmGmAfCmA | 532 | vmUpsfGpsmUmGmAfU | 74.8 | 76.3 |
| | | fUfUfUmUmUmGmGmAmUm | | mCmCmAmAmAmAmAf | | |
| | | CmAmCpsmA- | | UmGfUmCmCmUmAmG | | |
| | | GalNAc4psGalNAc4psGalNAc4 | | psmUpsmU | | |
| 159 | 602 | mUpsmUpsmGmGmUmGfAmA | 452 | vmApsfGpsmGmAmAfU | 61.8* | 91.7 |
| | | fGfUfUmUmUmUmCmAmUm | | mGmAmAmAmAmAmCf | | |
| | | UmCmCpsmU- | | UmUfCmAmCmCmAmA | | |
| | | GalNAc4psGalNAc4psGalNAc4 | | psmUpsmU | | |
| 160 | 603 | mApsmCpsmCmAmAmUfAmA | 516 | vmApsfApsmAmAmAfU | 44* | 88 |
| | | fGfAfAmAmAmUmGmAmUm | | mCmAmUmUmUmUmCf | | |
| | | UmUmUpsmU- | | UmUfAmUmUmGmGmU | | |
| | | GalNAc4psGalNAc4psGalNAc4 | | psmUpsmU | | |
| 161 | 595 | mUpsmGpsmGmUmAmUfCmA | 537 | vmApsfGpsmAmCmAfU | 64.9 | |
| | | fAfAfAmCmCmUmCmAmUm | | mGmAmGmGmUmUmUf | | |
| | | GmUmCmU- | | UmGfAmUmAmCmCmA | | |
| | | GalNAc4psGalNAc4psGalNAc4 | | psmUpsmU | | |
| 150 | 595 | mUpsmGpsmGmUmAmUfCmA | 623 | mApsfGpsmAmCmAfUm | 22.3 | |
| | | fAfAfAmCmCmUmCmAmUm | | GmAmGmGmUmUmUfU | | |
| | | GmUmCmU- | | mGfAmUmAmCmCmAps | | |
| | | GalNAc4psGalNAc4psGalNAc4 | | mUpsmU | | |
| 162 | 600 | mGpsmUpsmAmUmCmAfAmA | 538 | vmApsfGpsmAmGmAfC | 62.9 | |
| | | fAfCfCmUmCmAmUmGmUm | | mAmUmGmAmGmGmUf | | |
| | | CmUmCmU- | | UmUfUmGmAmUmAmC | | |
| | | GalNAc4psGalNAc4psGalNAc4 | | psmUpsmU | | |
| 152 | 597 | mGpsmUpsmAmUmCmAfAmA | 625 | mApsfGpsmAmGmAfCm | 49.7 | |
| | | fAfCfCmUmCmAmUmGmUm | | AmUmGmAmGmGmUfU | | |
| | | CmUmCmU- | | mUfUmGmAmUmAmCps | | |
| | | GalNAc4psGalNAc4psGalNAc4 | | mUpsmU | | |
| 132 | 577 | mUpsmGpsmGmAfGmUfCfGf | 605 | vmApsfApsmCmUmUfCm | 48.6 | |
| | | UmUmGmGmUmGmAmAmG | | AmCmCmAmAmCmGfA | | |
| | | mUmU- | | mCfUmCmCmApsmApsm | | |
| | | GalNAc4psGalNAc4psGalNAc4 | | G | | |
| 133 | 578 | mUpsmGpsmGmAfGmUfCfGf | 606 | mApsfApsmCmUmUfCm | 56.5* | |
| | | UmUmGmGmUmGmAmAmG | | AmCmCmAmAmCmGfA | | |
| | | mUmU- | | mCfUmCmCmApsmApsm | | |
| | | GalNAc4psGalNAc4psGalNAc4 | | G | | |
| 134 | 579 | mCpsmUpsmUmGmGmAfGmU | 607 | vmApsfApsmCmUmUfCm | 63.1 | |
| | | fCfGfUmUmGmGmUmGmAm | | AmCmCmAmAmCmGfA | | |
| | | AmGmUmU- | | mCfUmCmCmAmAmGps | | |
| | | GalNAc4psGalNAc4psGalNAc4 | | mUpsmA | | |
| 135 | 580 | mCpsmUpsmUmGmGmAfGmU | 608 | mApsfApsmCmUmUfCm | 57.1 | |
| | | fCfGfUmUmGmGmUmGmAm | | AmCmCmAmAmCmGfA | | |
| | | AmGmUmU- | | mCfUmCmCmAmAmGps | | |
| | | GalNAc4psGalNAc4psGalNAc4 | | mUpsmA | | |
| 137 | 582 | mGpsmGpsmUmGfAmAfGfUf | 610 | vmApsfGpsmGmAmAfU | 74.6* | 65.8 |
| | | UmUmUmUmCmAmUmUmC | | mGmAmAmAmAmAmCf | | |
| | | mCmU- | | UmUfCmAmCmCpsmAps | | |
| | | GalNAc4psGalNAc4psGalNAc4 | | mA | | |
| 138 | 583 | mGpsmGpsmUmGfAmAfGfUf | 611 | mApsfGpsmGmAmAfUm | 45.7 | |
| | | UmUmUmUmCmAmUmUmC | | GmAmAmAmAmAmCfU | | |
| | | mCmU- | | mUfCmAmCmCpsmApsm | | |
| | | GalNAc4psGalNAc4psGalNAc4 | | A | | |
| 139 | 584 | mUpsmUpsmGmGmUmGfAmA | 612 | vmApsfGpsmGmAmAfU | 64.5 | |
| | | fGfUfUmUmUmUmCmAmUm | | mGmAmAmAmAmAmCf | | |
| | | UmCmCmU- | | UmUfCmAmCmCmAmA | | |
| | | GalNAc4psGalNAc4psGalNAc4 | | psmCpsmG | | |
| 140 | 585 | mUpsmUpsmGmGmUmGfAmA | 613 | mApsfGpsmGmAmAfUm | 49.4 | |
| | | fGfUfUmUmUmUmCmAmUm | | GmAmAmAmAmAmCfU | | |
| | | UmCmCmU- | | mUfCmAmCmCmAmAps | | |
| | | GalNAc4psGalNAc4psGalNAc4 | | mCpsmG | | |
| 141 | 586 | mUpsmApsmUmUmGmGfUmU | 614 | mUpsfApsmAmUmAfUm | 16.3 | |
| | | fCfUfGmUmGmGmGmAmUm | | CmCmCmAmCmAmGfA | | |
| | | AmUmUmA- | | mAfCmCmAmAmUmAps | | |
| | | GalNAc4psGalNAc4psGalNAc4 | | mUpsmG | | |
| 142 | 587 | mUpsmApsmUmUmGmGfUmU | 615 | vmUpsfApsmAmUmAfU | 35 | |
| | | fCfUfGmUmGmGmGmAmUm | | mCmCmCmAmCmAmGf | | |
| | | AmUmUmA- | | AmAfCmCmAmAmUmA | | |
| | | GalNAc4psGalNAc4psGalNAc4 | | psmUpsmG | | |
| 143 | 588 | mUpsmApsmUmUmGmGfUmU | 616 | vmApsfApsmAmUmAfU | 50.6 | |
| | | fCfUfGmUmGmGmGmAmUm | | mCmCmCmAmCmAmGf | | |
| | | AmUmUmA- | | AmAfCmCmAmAmUmA | | |
| | | GalNAc4psGalNAc4psGalNAc4 | | psmUpsmG | | |
| 144 | 589 | mUpsmUpsmGmGmGmAfAmA | 617 | mUpsfUpsmUmGmAfUm | 37.4 | |
| | | fAfAfCmUmGmGmUmAmUm | | AmCmCmAmGmUmUfU | | |
| | | CmAmAmA- | | mUfUmCmCmCmAmAps | | |
| | | GalNAc4psGalNAc4psGalNAc4 | | mGpsmG | | |
| 145 | 590 | mUpsmUpsmGmGmGmAfAmA | 618 | vmUpsfUpsmUmGmAfU | 33.4 | |
| | | fAfAfCmUmGmGmUmAmUm | | mAmCmCmAmGmUmUf | | |
| | | CmAmAmA- | | UmUfUmCmCmCmAmA | | |
| | | GalNAc4psGalNAc4psGalNAc4 | | psmGpsmG | | |
| 146 | 591 | mUpsmUpsmGmGmGmAfAmA | 619 | vmApsfUpsmUmGmAfU | 35.8 | |
| | | fAfAfCmUmGmGmUmAmUm | | mAmCmCmAmGmUmUf | | |
| | | CmAmAmA- | | UmUfUmCmCmCmAmA | | |
| | | GalNAc4psGalNAc4psGalNAc4 | | psmGpsmG | | |
| 147 | 592 | mApsmCpsmCmAmAmUfAmA | 620 | mApsfApsmAmAmAfUm | 21.4 | |
| | | fGfAfAmAmAmUmGmAmUm | | CmAmUmUmUmUmCfU | | |
| | | UmUmUmU- | | mUfAmUmUmGmGmUps | | |
| | | GalNAc4psGalNAc4psGalNAc4 | | mApsmA | | |
| 148 | 593 | mApsmCpsmCmAmAmUfAmA | 621 | vmApsfApsmAmAmAfU | 52.0 | 69.9 |
| | | fGfAfAmAmAmUmGmAmUm | | mCmAmUmUmUmUmCf | | |
| | | UmUmUmU- | | UmUfAmUmUmGmGmU | | |
| | | GalNAc4psGalNAc4psGalNAc4 | | psmApsmA | | |
| 149 | 594 | mApsmCpsmCmAmAmUfAmA | 622 | vmUpsfApsmAmAmAfU | 45.5 | |
| | | fGfAfAmAmAmUmGmAmUm | | mCmAmUmUmUmUmCf | | |
| | | UmUmUmU- | | UmUfAmUmUmGmGmU | | |
| | | GalNAc4psGalNAc4psGalNAc4 | | psmApsmA | | |
| 153 | 598 | mGpsmUpsmAmUmCmAfAmA | 626 | mApsfGpsmAmGmAfCm | 61.6 | |
| | | fAfCfCmUmCmAmUmGmUm | | AmUmGmAmGmGmUfU | | |
| | | CmUmCmU- | | mUfUmGmAmUmAmCps | | |
| | | GalNAc4psGalNAc4psGalNAc4 | | mCpsmA | | |
| 154 | 599 | mGpsmUpsmAmUmCmAfAmA | 627 | vmApsfGpsmAmGmAfC | 70.3 | |
| | | fAfCfCmUmCmAmUmGmUm | | mAmUmGmAmGmGmUf | | |
| | | CmUmCmU- | | UmUfUmGmAmUmAmC | | |
| | | GalNAc4psGalNAc4psGalNAc4 | | psmCpsmA | | |
| 155 | 600 | mGpsmUpsmAmUmCmAfAmA | 628 | vmUpsfGpsmAmGmAfC | 53.2 | |
| | | fAfCfCmUmCmAmUmGmUm | | mAmUmGmAmGmGmUf | | |
| | | CmUmCmU- | | UmUfUmGmAmUmAmC | | |
| | | GalNAc4psGalNAc4psGalNAc4 | | psmCpsmA | | |
| 144 | 589 | mUpsmUpsmGmGmGmAfAmA | 617 | mUpsfUpsmUmGmAfUm | | 49.3 |
| | | fAfAfCmUmGmGmUmAmUm | | AmCmCmAmGmUmUfU | | |
| | | CmAmAmA- | | mUfUmCmCmCmAmAps | | |
| | | GalNAc4psGalNAc4psGalNAc4 | | mGpsmG | | |
| 151 | 596 | mUpsmGpsmGmUmAmUfCmA | 624 | vmApsfGpsmAmCmAfU | 53.6 | 73.6 |
| | | fAfAfAmCmCmUmCmAmUm | | mGmAmGmGmUmUmUf | | |
| | | GmUmCmU- | | UmGfAmUmAmCmCmA | | |
| | | GalNAc4psGalNAc4psGalNAc4 | | psmGpsmU | | |
| 164 | 582 | mGpsmGpsmUmGfAmAfGfUf | 630 | vmApsfGpsmGmAmAfUu | 7.6 | −37.1 |
| | | UmUmUmUmCmAmUmUmC | | nGmAmAmAmAmAmCf | | |
| | | mCmU- | | UmUfCmAmCmCpsmAps | | |
| | | GalNAc4psGalNAc4psGalNAc4 | | mA | | |
| 165 | 582 | mGpsmGpsmUmGfAmAfGfUf | 631 | vmApsfGpsmGmAmAfU | −21.8 | −48.2 |
| | | UmUmUmUmCmAmUmUmC | | mun34GmAmAmAmAmA | | |
| | | mCmU- | | mCfUmUfCmAmCmCpsm | | |
| | | GalNAc4psGalNAc4psGalNAc4 | | ApsmA | | |
| 166 | 582 | mGpsmGpsmUmGfAmAfGfUf | 632 | v- | 47 | 60 |
| | | UmUmUmUmCmAmUmUmC | | munUpsfGpsmGmAmAfU | | |
| | | mCmU- | | mGmAmAmAmAmAmCf | | |
| | | GalNAc4psGalNAc4psGalNAc4 | | UmUfCmAmCmCpsmAps | | |
| | | | | mA | | |
| 167 | 582 | mGpsmGpsmUmGfAmAfGfUf | 633 | 4hUpsfGpsmGmAmAfUm | 32 | 44 |
| | | UmUmUmUmCmAmUmUmC | | GmAmAmAmAmAmCfU | | |
| | | mCmU- | | mUfCmAmCmCpsmApsm | | |
| | | GalNAc4psGalNAc4psGalNAc4 | | A | | |
| 168 | 582 | mGpsmGpsmUmGfAmAfGfUf | 634 | c2o- | 25 | 50 |
| | | UmUmUmUmCmAmUmUmC | | 4hUpsfGpsmGmAmAfUm | | |
| | | mCmU- | | GmAmAmAmAmAmCfU | | |
| | | GalNAc4psGalNAc4psGalNAc4 | | mUfCmAmCmCpsmApsm | | |
| | | | | A | | |
| 169 | 592 | mApsmCpsmCmAmAmUfAmA | 635 | v- | | 52 |
| | | fGfAfAmAmAmUmGmAmUm | | munUpsfApsmAmAmAfU | | |
| | | UmUmUmU- | | mCmAmUmUmUmUmCf | | |
| | | GalNAc4psGalNAc4psGalNAc4 | | UmUfAmUmUmGmGmU | | |
| | | | | psmApsmA | | |
| 170 | 592 | mApsmCpsmCmAmAmUfAmA | 636 | 4hUpsfApsmAmAmAfUm | | 51 |
| | | fGfAfAmAmAmUmGmAmUm | | CmAmUmUmUmUmCfU | | |
| | | UmUmUmU- | | mUfAmUmUmGmGmUps | | |
| | | GalNAc4psGalNAc4psGalNAc4 | | mApsmA | | |
| 171 | 592 | mApsmCpsmCmAmAmUfAmA | 637 | c2o- | | 51 |
| | | fGfAfAmAmAmUmGmAmUm | | 4hUpsfApsmAmAmAfUm | | |
| | | UmUmUmU- | | CmAmUmUmUmUmCfU | | |
| | | GalNAc4psGalNAc4psGalNAc4 | | mUfAmUmUmGmGmUps | | |
| | | | | mApsmA | | |
| 172 | 638 | mApsmCpsmCmAmAmUfAmA | 621 | vmApsfApsmAmAmAfU | 55 | 69 |
| | | fGfAfAmAmAmUmGmAmUm | | mCmAmUmUmUmUmCf | | |
| | | UmUmUpsmU- | | UmUfAmUmUmGmGmU | | |
| | | GalNAc4psGalNAc4psGalNAc4 | | psmApsmA | | |
| 173 | 595 | mUpsmGpsmGmUmAmUfCmA | 639 | vmApsfGpsmAmCmAfUu | 32.9 | 71.8 |
| | | fAfAfAmCmCmUmCmAmUm | | nGmAmGmGmUmUmUf | | |
| | | GmUmCmU- | | UmGfAmUmAmCmCmA | | |
| | | GalNAc4psGalNAc4psGalNAc4 | | psmGpsmU | | |
| 174 | 595 | mUpsmGpsmGmUmAmUfCmA | 640 | vmApsfGpsmAmCmAfU | 32.6 | 74.5 |
| | | fAfAfAmCmCmUmCmAmUm | | mun34GmAmGmGmUmU | | |
| | | GmUmCmU- | | mUfUmGfAmUmAmCmC | | |
| | | GalNAc4psGalNAc4psGalNAc4 | | mApsmGpsmU | | |

*indicates an average over more than one trial

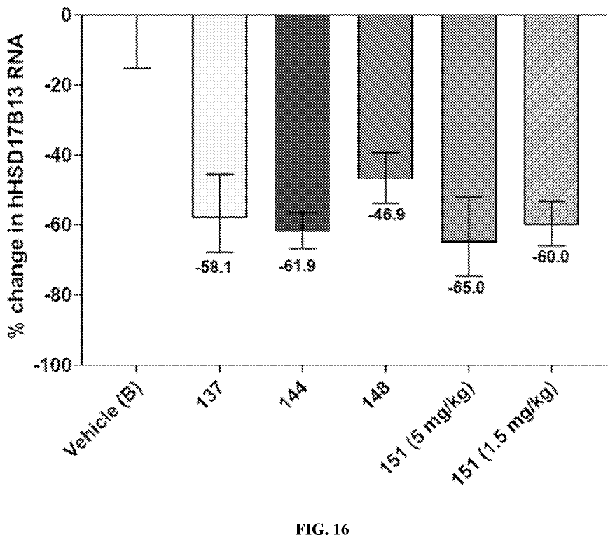

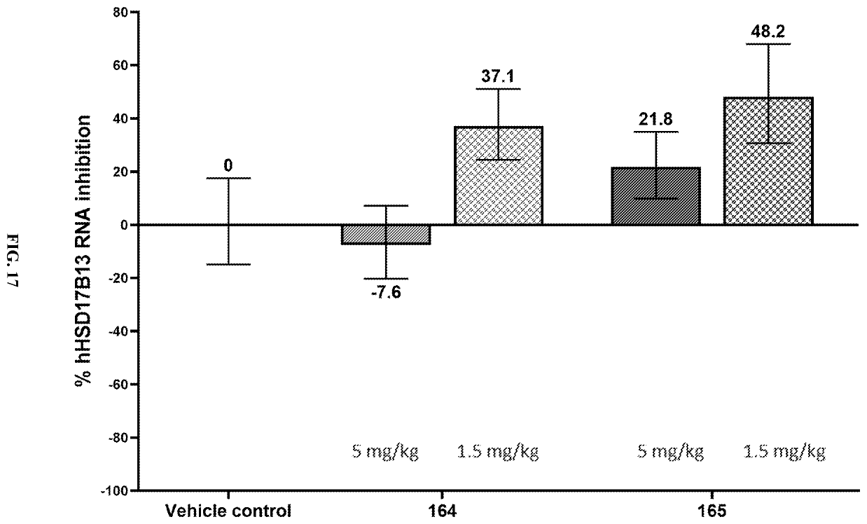

Protein knockdown for several duplexes was also assessed by western blot after 7 days (FIG. 11) and quantified (FIG. 12 and Table 5) and 14 days (FIG. 14) and quantified (FIG. 15 and Table 6). In addition, HSD17B13 mRNA knockdown results after 14 days for some duplexes are shown in FIG. 16 and Table 7.

TABLE 5
In Vivo Study-7 Days KD-Protein

| | | | | | % Protein | |
|---|---|---|---|---|---|---|
| mds- | SEQ | Sense Strand Base | SEQ | Antisense Strand Base | Inhibition (Drug v. | |
| siNA | ID | Sequence + Modifications | ID | Sequence + Modifications | Vehicle) | |
| (MD#) | NO. | (5′-3′) | NO. | (5′-3′) | 1.5 mg/kg | 5 mg/kg |
| 137 | 582 | mGpsmGpsmUmGfAmAfGfUfUmU | 610 | vmApsfGpsmGmAmAfU | | 67.26 |
| | | mUmUmCmAmUmUmCmCmU- | | mGmAmAmAmAmAmCf | | |
| | | GalNAc4psGalNAc4psGalNAc4 | | UmUfCmAmCmCpsmAps | | |
| | | | | mA | | |
| 144 | 589 | mUpsmUpsmGmGmGmAfAmAfAf | 617 | mUpsfUpsmUmGmAfUm | | 32.53 |
| | | AfCmUmGmGmUmAmUmCmAmA | | AmCmCmAmGmUmUfU | | |
| | | mA-GalNAc4psGalNAc4psGalNAc4 | | mUfUmCmCmCmAmAps | | |
| | | | | mGpsmG | | |
| 148 | 593 | mApsmCpsmCmAmAmUfAmAfGfA | 621 | vmApsfApsmAmAmAfU | | 46.6 |
| | | fAmAmAmUmGmAmUmUmUmUm | | mCmAmUmUmUmUmCf | | |
| | | U-GalNAc4psGalNAc4psGalNAc4 | | UmUfAmUmUmGmGmU | | |
| | | | | psmApsmA | | |
| 151 | 596 | mUpsmGpsmGmUmAmUfCmAfAf | 624 | vmApsfGpsmAmCmAfU | 55.26 | 87.07 |
| | | AfAmCmCmUmCmAmUmGmUmC | | mGmAmGmGmUmUmUf | | |
| | | mU-GalNAc4psGalNAc4psGalNAc4 | | UmGfAmUmAmCmCmA | | |
| | | | | psmGpsmU | | |

TABLE 6
In Vivo Study-14 Days KD-Protein

| | | | | | % Protein | |
|---|---|---|---|---|---|---|
| | SEQ | Sense Strand Base | SEQ | Antisense Strand Base | Inhibition (Drug v. | |
| mds- | ID | Sequence + Modifications | ID | Sequence + Modifications | Vehicle) | |
| siNA | NO. | (5′-3′) | NO. | (5′-3′) | 1.5 mg/kg | 5 mg/kg |
| 137 | 582 | mGpsmGpsmUmGfAmAfGfUfUmU | 610 | vmApsfGpsmGmAmAfU | | 86.1 |
| | | mUmUmCmAmUmUmCmCmU- | | mGmAmAmAmAmAmCf | | |
| | | GalNAc4psGalNAc4psGalNAc4 | | UmUfCmAmCmCpsmAps | | |
| | | | | mA | | |
| 144 | 589 | mUpsmUpsmGmGmGmAfAmAfAf | 617 | mUpsfUpsmUmGmAfUm | | 80.5 |
| | | AfCmUmGmGmUmAmUmCmAmA | | AmCmCmAmGmUmUfU | | |
| | | mA-GalNAc4psGalNAc4psGalNAc4 | | mUfUmCmCmCmAmAps | | |
| | | | | mGpsmG | | |
| 148 | 593 | mApsmCpsmCmAmAmUfAmAfGfA | 621 | vmApsfApsmAmAmAfU | | 62 |
| | | fAmAmAmUmGmAmUmUmUmUm | | mCmAmUmUmUmUmCf | | |
| | | U-GalNAc4psGalNAc4psGalNAc4 | | UmUfAmUmUmGmGmU | | |
| | | | | psmApsmA | | |
| 151 | 596 | mUpsmGpsmGmUmAmUfCmAfAf | 624 | vmApsfGpsmAmCmAfU | 81.3 | 84.7 |
| | | AfAmCmCmUmCmAmUmGmUmC | | mGmAmGmGmUmUmUf | | |
| | | mU-GalNAc4psGalNAc4psGalNAc4 | | UmGfAmUmAmCmCmA | | |
| | | | | psmGpsmU | | |

TABLE 7
In Vivo Study-14 Days KD-mRNA

| | SEQ | Sense Strand Base | SEQ | Antisense Strand Base | % RNA Inhibition | |
|---|---|---|---|---|---|---|
| mds- | ID | Sequence + Modifications | ID | Sequence + Modifications | (Drug v. Vehicle) | |
| siNA | NO. | (5′-3′) | NO. | (5′-3′) | 1.5 mg/kg | 5 mg/kg |
| 137 | 582 | mGpsmGpsmUmGfAmAfGfUfUmU | 610 | vmApsfGpsmGmAmAfU | | 58.1 |
| | | mUmUmCmAmUmUmCmCmU- | | mGmAmAmAmAmAmCf | | |
| | | GalNAc4psGalNAc4psGalNAc4 | | UmUfCmAmCmCpsmAps | | |
| | | | | mA | | |
| 144 | 589 | mUpsmUpsmGmGmGmAfAmAfAf | 617 | mUpsfUpsmUmGmAfUm | | 61.9 |
| | | AfCmUmGmGmUmAmUmCmAmA | | AmCmCmAmGmUmUfU | | |
| | | mA-GalNAc4psGalNAc4psGalNAc4 | | mUfUmCmCmCmAmAps | | |
| | | | | mGpsmG | | |
| 148 | 593 | mApsmCpsmCmAmAmUfAmAfGfA | 621 | vmApsfApsmAmAmAfU | | 46.9 |
| | | fAmAmAmUmGmAmUmUmUmUm | | mCmAmUmUmUmUmCf | | |
| | | U-GalNAc4psGalNAc4psGalNAc4 | | UmUfAmUmUmGmGmU | | |
| | | | | psmApsmA | | |
| 151 | 596 | mUpsmGpsmGmUmAmUfCmAfAfA | 624 | vmApsfGpsmAmCmAfU | 60 | 65 |
| | | fAmCmCmUmCmAmUmGmUmCm | | mGmAmGmGmUmUmUf | | |
| | | U-GalNAc4psGalNAc4psGalNAc4 | | UmGfAmUmAmCmCmA | | |
| | | | | psmGpsmU | | |

TABLE 8
21-mers with 5′-VP in AS

| | SEQ | Sense/ | | | SEQ | |
|---|---|---|---|---|---|---|
| | ID | Anti- | | | ID | |
| D# | NO. | sense | Base Sequence (5′-3′) | MD# | NO. | Modified Sequence (5′-3′) |
| 1 | 1 | Sense | CAUCAUCUACUCC | 1 | 316 | mCpsmApsmUmCmAmUfCmU |
| | | | UACUUGGA | | | fAfCfUmCmCmUmAmCmUmU |
| | | | | | | mGmGpsmA |
| | 101 | Anti- | UCCAAGUAGGAGU | | 446 | vmUpsfCpsmCmAmAfGmUmA |
| | | Sense | AGAUGAUGUU | | | mGmGmAmGmUfAmGfAmUm |
| | | | | | | GmAmUmGpsmUpsmU |
| 2 | 2 | Sense | CUACUCCUACUUG | 2 | 317 | mCpsmUpsmAmCmUmCfCmUf |
| | | | GAGUCGUU | | | AfCfUmUmGmGmAmGmUmC |
| | | | | | | mGmUpsmU |
| | 102 | Anti- | AACGACUCCAAGU | | 447 | vmApsfApsmCmGmAfCmUmC |
| | | Sense | AGGAGUAGUU | | | mCmAmAmGmUfAmGfGmAm |
| | | | | | | GmUmAmGpsmUpsmU |
| 3 | 3 | Sense | CCUACUUGGAGUC | 3 | 318 | mCpsmCpsmUmAmCmUfUmG |
| | | | GUUGGUGA | | | fGfAfGmUmCmGmUmUmGm |
| | | | | | | GmUmGpsmA |
| | 103 | Anti- | UCACCAACGACUC | | 448 | vmUpsfCpsmAmCmCfAmAmC |
| | | Sense | CAAGUAGGUU | | | mGmAmCmUmCfCmAfAmGm |
| | | | | | | UmAmGmGpsmUpsmU |
| 4 | 4 | Sense | CUACUUGGAGUCG | 4 | 319 | mCpsmUpsmAmCmUmUfGmG |
| | | | UUGGUGAA | | | fAfGfUmCmGmUmUmGmGm |
| | | | | | | UmGmApsmA |
| | 104 | Anti- | UUCACCAACGACU | | 449 | vmUpsfUpsmCmAmCfCmAmA |
| | | Sense | CCAAGUAGUU | | | mCmGmAmCmUfCmCfAmAm |
| | | | | | | GmUmAmGpsmUpsmU |
| 5 | 5 | Sense | CUUGGAGUCGUUG | 5 | 320 | mCpsmUpsmUmGmGmAfGmU |
| | | | GUGAAGUU | | | fCfGfUmUmGmGmUmGmAm |
| | | | | | | AmGmUpsmU |
| | 105 | Anti- | AACUUCACCAACG | | 450 | vmApsfApsmCmUmUfCmAmC |
| | | Sense | ACUCCAAGUU | | | mCmAmAmCmGfAmCfUmCm |
| | | | | | | CmAmAmGpsmUpsmU |
| 6 | 6 | Sense | UGGAGUCGUUGGU | 6 | 321 | mUpsmGpsmGmAmGmUfCmG |
| | | | GAAGUUUU | | | fUfUfGmGmUmGmAmAmGm |
| | | | | | | UmUmUpsmU |
| | 106 | Anti- | AAAACUUCACCAA | | 451 | vmApsfApsmAmAmCfUmUmC |
| | | Sense | CGACUCCAUU | | | mAmCmCmAmAfCmGfAmCm |
| | | | | | | UmCmCmApsmUpsmU |
| 7 | 7 | Sense | UUGGUGAAGUUUU | 7 | 322 | mUpsmUpsmGmGmUmGfAmA |
| | | | UCAUUCCU | | | fGfUfUmUmUmUmCmAmUm |
| | | | | | | UmCmCpsmU |
| | 107 | Anti- | AGGAAUGAAAAAC | | 452 | vmApsfGpsmGmAmAfUmGmA |
| | | Sense | UUCACCAAUU | | | mAmAmAmAmCfUmUfCmAm |
| | | | | | | CmCmAmApsmUpsmU |
| 8 | 8 | Sense | GGUGAAGUUUUUC | 8 | 323 | mGpsmGpsmUmGmAmAfGmU |
| | | | AUUCCUCA | | | fUfUfUmUmCmAmUmUmCm |
| | | | | | | CmUmCpsmA |
| | 108 | Anti- | UGAGGAAUGAAAA | | 453 | vmUpsfGpsmAmGmGfAmAmU |
| | | Sense | ACUUCACCUU | | | mGmAmAmAmAfAmCfUmUm |
| | | | | | | CmAmCmCpsmUpsmU |
| 9 | 9 | Sense | GGAAUAGGCAGGC | 9 | 324 | mGpsmGpsmAmAmUmAfGmG |
| | | | AGACUACU | | | fCfAfGmGmCmAmGmAmCm |
| | | | | | | UmAmCpsmU |
| | 109 | Anti- | AGUAGUCUGCCUG | | 454 | vmApsfGpsmUmAmGfUmCmU |
| | | Sense | CCUAUUCCUU | | | mGmCmCmUmGfCmCfUmAm |
| | | | | | | UmUmCmCpsmUpsmU |
| 10 | 10 | Sense | GAAUAGGCAGGCA | 10 | 325 | mGpsmApsmAmUmAmGfGmC |
| | | | GACUACUU | | | fAfGfGmCmAmGmAmCmUm |
| | | | | | | AmCmUpsmU |
| | 110 | Anti- | AAGUAGUCUGCCU | | 455 | vmApsfApsmGmUmAfGmUmC |
| | | Sense | GCCUAUUCUU | | | mUmGmCmCmUfGmCfCmUm |
| | | | | | | AmUmUmCpsmUpsmU |
| 11 | 11 | Sense | AAUAGGCAGGCAG | 11 | 326 | mApsmApsmUmAmGmGfCmA |
| | | | ACUACUUA | | | fGfGfCmAmGmAmCmUmAm |
| | | | | | | CmUmUpsmA |
| | 111 | Anti- | UAAGUAGUCUGCC | | 456 | vmUpsfApsmAmGmUfAmGmU |
| | | Sense | UGCCUAUUUU | | | mCmUmGmCmCfUmGfCmCm |
| | | | | | | UmAmUmUpsmUpsmU |
| 12 | 12 | Sense | AUAGGCAGGCAGA | 12 | 327 | mApsmUpsmAmGmGmCfAmG |
| | | | CUACUUAU | | | fGfCfAmGmAmCmUmAmCm |
| | | | | | | UmUmApsmU |
| | 112 | Anti- | AUAAGUAGUCUGC | | 457 | vmApsfUpsmAmAmGfUmAmG |
| | | Sense | CUGCCUAUUU | | | mUmCmUmGmCfCmUfGmCm |
| | | | | | | CmUmAmUpsmUpsmU |
| 13 | 13 | Sense | AGGCAGGCAGACU | 13 | 328 | mApsmGpsmGmCmAmGfGmC |
| | | | ACUUAUGA | | | fAfGfAmCmUmAmCmUmUm |
| | | | | | | AmUmGpsmA |
| | 113 | Anti- | UCAUAAGUAGUCU | | 458 | vmUpsfCpsmAmUmAfAmGmU |
| | | Sense | GCCUGCCUUU | | | mAmGmUmCmUfGmCfCmUm |
| | | | | | | GmCmCmUpsmUpsmU |
| 14 | 14 | Sense | GGCAGGCAGACUA | 14 | 329 | mGpsmGpsmCmAmGmGfCmA |
| | | | CUUAUGAA | | | fGfAfCmUmAmCmUmUmAm |
| | | | | | | UmGmApsmA |
| | 114 | Anti- | UUCAUAAGUAGUC | | 459 | vmUpsfUpsmCmAmUfAmAmG |
| | | Sense | UGCCUGCCUU | | | mUmAmGmUmCfUmGfCmCm |
| | | | | | | UmGmCmCpsmUpsmU |
| 15 | 15 | Sense | UAUUGGUUCUGUG | 15 | 330 | mUpsmApsmUmUmGmGfUmU |
| | | | GGAUAUUA | | | fCfUfGmUmGmGmGmAmUm |
| | | | | | | AmUmUpsmA |
| | 115 | Anti- | UAAUAUCCCACAG | | 460 | vmUpsfApsmAmUmAfUmCmC |
| | | Sense | AACCAAUAUU | | | mCmAmCmAmGfAmAfCmCm |
| | | | | | | AmAmUmApsmUpsmU |
| 16 | 16 | Sense | UUGGUUCUGUGGG | 16 | 331 | mUpsmUpsmGmGmUmUfCmU |
| | | | AUAUUAAU | | | fGfUfGmGmGmAmUmAmUm |
| | | | | | | UmAmApsmU |
| | 116 | Anti- | AUUAAUAUCCCAC | | 461 | vmApsfUpsmUmAmAfUmAmU |
| | | Sense | AGAACCAAUU | | | mCmCmCmAmCfAmGfAmAm |
| | | | | | | CmCmAmApsmUpsmU |
| 17 | 17 | Sense | GGUUCUGUGGGAU | 17 | 332 | mGpsmGpsmUmUmCmUfGmU |
| | | | AUUAAUAA | | | fGfGfGmAmUmAmUmUmAm |
| | | | | | | AmUmApsmA |
| | 117 | Anti- | UUAUUAAUAUCCC | | 462 | vmUpsfUpsmAmUmUfAmAmU |
| | | Sense | ACAGAACCUU | | | mAmUmCmCmCfAmCfAmGm |
| | | | | | | AmAmCmCpsmUpsmU |
| 18 | 18 | Sense | AUGUGGUAGACUG | 18 | 333 | mApsmUpsmGmUmGmGfUmA |
| | | | CAGCAACA | | | fGfAfCmUmGmCmAmGmCm |
| | | | | | | AmAmCpsmA |
| | 118 | Anti- | UGUUGCUGCAGUC | | 463 | vmUpsfGpsmUmUmGfCmUmG |
| | | Sense | UACCACAUUU | | | mCmAmGmUmCfUmAfCmCm |
| | | | | | | AmCmAmUpsmUpsmU |
| 19 | 19 | Sense | CUGCAGCAACAGA | 19 | 334 | mCpsmUpsmGmCmAmGfCmA |
| | | | GAAGAGAU | | | fAfCfAmGmAmGmAmAmGm |
| | | | | | | AmGmApsmU |
| | 119 | Anti- | AUCUCUUCUCUGU | | 464 | vmApsfUpsmCmUmCfUmUmC |
| | | Sense | UGCUGCAGUU | | | mUmCmUmGmUfUmGfCmUm |
| | | | | | | GmCmAmGpsmUpsmU |
| 20 | 20 | Sense | AAAUCAGGUGAAG | 20 | 335 | mApsmApsmAmUmCmAfGmG |
| | | | AAAGAAGU | | | fUfGfAmAmGmAmAmAmGm |
| | | | | | | AmAmGpsmU |
| | 120 | Anti- | ACUUCUUUCUUCA | | 465 | vmApsfCpsmUmUmCfUmUmU |
| | | Sense | CCUGAUUUUU | | | mCmUmUmCmAfCmCfUmGm |
| | | | | | | AmUmUmUpsmUpsmU |
| 21 | 21 | Sense | GAAGAAAGAAGUG | 21 | 336 | mGpsmApsmAmGmAmAfAmG |
| | | | GGUGAUGU | | | fAfAfGmUmGmGmGmUmGm |
| | | | | | | AmUmGpsmU |
| | 121 | Anti- | ACAUCACCCACUU | | 466 | vmApsfCpsmAmUmCfAmCmC |
| | | Sense | CUUUCUUCUU | | | mCmAmCmUmUfCmUfUmUm |
| | | | | | | CmUmUmCpsmUpsmU |
| 22 | 22 | Sense | CCAAGGAUGAAGA | 22 | 337 | mCpsmCpsmAmAmGmGfAmU |
| | | | GAUUACCA | | | fGfAfAmGmAmGmAmUmUm |
| | | | | | | AmCmCpsmA |
| | 122 | Anti- | UGGUAAUCUCUUC | | 467 | vmUpsfGpsmGmUmAfAmUmC |
| | | Sense | AUCCUUGGUU | | | mUmCmUmUmCfAmUfCmCm |
| | | | | | | UmUmGmGpsmUpsmU |
| 23 | 23 | Sense | GAAGAGAUUACCA | 23 | 338 | mGpsmApsmAmGmAmGfAmU |
| | | | AGACAUUU | | | fUfAfCmCmAmAmGmAmCm |
| | | | | | | AmUmUpsmU |
| | 123 | Anti- | AAAUGUCUUGGUA | | 468 | vmApsfApsmAmUmGfUmCmU |
| | | Sense | AUCUCUUCUU | | | mUmGmGmUmAfAmUfCmUm |
| | | | | | | CmUmUmCpsmUpsmU |
| 24 | 24 | Sense | AGAGAUUACCAAG | 24 | 339 | mApsmGpsmAmGmAmUfUmA |
| | | | ACAUUUGA | | | fCfCfAmAmGmAmCmAmUm |
| | | | | | | UmUmGpsmA |
| | 124 | Anti- | UCAAAUGUCUUGG | | 469 | vmUpsfCpsmAmAmAfUmGmU |
| | | Sense | UAAUCUCUUU | | | mCmUmUmGmGfUmAfAmUm |
| | | | | | | CmUmCmUpsmUpsmU |
| 25 | 25 | Sense | GAUUACCAAGACA | 25 | 340 | mGpsmApsmUmUmAmCfCmA |
| | | | UUUGAGGU | | | fAfGfAmCmAmUmUmUmGm |
| | | | | | | AmGmGpsmU |
| | 125 | Anti- | ACCUCAAAUGUCU | | 470 | vmApsfCpsmCmUmCfAmAmA |
| | | Sense | UGGUAAUCUU | | | mUmGmUmCmUfUmGfGmUm |
| | | | | | | AmAmUmCpsmUpsmU |
| 26 | 26 | Sense | UACCAAGACAUUU | 26 | 341 | mUpsmApsmCmCmAmAfGmA |
| | | | GAGGUCAA | | | fCfAfUmUmUmGmAmGmGm |
| | | | | | | UmCmApsmA |
| | 126 | Anti- | UUGACCUCAAAUG | | 471 | vmUpsfUpsmGmAmCfCmUmC |
| | | Sense | UCUUGGUAUU | | | mAmAmAmUmGfUmCfUmUm |
| | | | | | | GmGmUmApsmUpsmU |
| 27 | 27 | Sense | CCAAGACAUUUGA | 27 | 342 | mCpsmCpsmAmAmGmAfCmA |
| | | | GGUCAACA | | | fUfUfUmGmAmGmGmUmCm |
| | | | | | | AmAmCpsmA |
| | 127 | Anti- | UGUUGACCUCAAA | | 472 | vmUpsfGpsmUmUmGfAmCmC |
| | | Sense | UGUCUUGGUU | | | mUmCmAmAmAfUmGfUmCm |
| | | | | | | UmUmGmGpsmUpsmU |
| 28 | 28 | Sense | UGAGGUCAACAUC | 28 | 343 | mUpsmGpsmAmGmGmUfCmA |
| | | | CUAGGACA | | | fAfCfAmUmCmCmUmAmGm |
| | | | | | | GmAmCpsmA |
| | 128 | Anti- | UGUCCUAGGAUGU | | 473 | vmUpsfGpsmUmCmCfUmAmG |
| | | Sense | UGACCUCAUU | | | mGmAmUmGmUfUmGfAmCm |
| | | | | | | CmUmCmApsmUpsmU |
| 29 | 29 | Sense | AGGUCAACAUCCU | 29 | 344 | mApsmGpsmGmUmCmAfAmC |
| | | | AGGACAUU | | | fAfUfCmCmUmAmGmGmAm |
| | | | | | | CmAmUpsmU |
| | 129 | Anti- | AAUGUCCUAGGAU | | 474 | vmApsfApsmUmGmUfCmCmU |
| | | Sense | GUUGACCUUU | | | mAmGmGmAmUfGmUfUmGm |
| | | | | | | AmCmCmUpsmUpsmU |
| 30 | 30 | Sense | GUCAACAUCCUAG | 30 | 345 | mGpsmUpsmCmAmAmCfAmU |
| | | | GACAUUUU | | | fCfCfUmAmGmGmAmCmAm |
| | | | | | | UmUmUpsmU |
| | 130 | Anti- | AAAAUGUCCUAGG | | 475 | vmApsfApsmAmAmUfGmUmC |
| | | Sense | AUGUUGACUU | | | mCmUmAmGmGfAmUfGmUm |
| | | | | | | UmGmAmCpsmUpsmU |
| 31 | 31 | Sense | UCAACAUCCUAGG | 31 | 346 | mUpsmCpsmAmAmCmAfUmC |
| | | | ACAUUUUU | | | fCfUfAmGmGmAmCmAmUm |
| | | | | | | UmUmUpsmU |
| | 131 | Anti- | AAAAAUGUCCUAG | | 476 | vmApsfApsmAmAmAfUmGmU |
| | | Sense | GAUGUUGAUU | | | mCmCmUmAmGfGmAfUmGm |
| | | | | | | UmUmGmApsmUpsmU |
| 32 | 32 | Sense | CAUCCUAGGACAU | 32 | 347 | mCpsmApsmUmCmCmUfAmG |
| | | | UUUUGGAU | | | fGfAfCmAmUmUmUmUmUm |
| | | | | | | GmGmApsmU |
| | 132 | Anti- | AUCCAAAAAUGUC | | 477 | vmApsfUpsmCmCmAfAmAmA |
| | | Sense | CUAGGAUGUU | | | mAmUmGmUmCfCmUfAmGm |
| | | | | | | GmAmUmGpsmUpsmU |
| 33 | 33 | Sense | UCCUAGGACAUUU | 33 | 348 | mUpsmCpsmCmUmAmGfGmA |
| | | | UUGGAUCA | | | fCfAfUmUmUmUmUmGmGm |
| | | | | | | AmUmCpsmA |
| | 133 | Anti- | UGAUCCAAAAAUG | | 478 | vmUpsfGpsmAmUmCfCmAmA |
| | | Sense | UCCUAGGAUU | | | mAmAmAmUmGfUmCfCmUm |
| | | | | | | AmGmGmApsmUpsmU |
| 34 | 34 | Sense | AGGACAUUUUUGG | 34 | 349 | mApsmGpsmGmAmCmAfUmU |
| | | | AUCACAAA | | | fUfUfUmGmGmAmUmCmAm |
| | | | | | | CmAmApsmA |
| | 134 | Anti- | UUUGUGAUCCAAA | | 479 | vmUpsfUpsmUmGmUfGmAmU |
| | | Sense | AAUGUCCUUU | | | mCmCmAmAmAfAmAfUmGm |
| | | | | | | UmCmCmUpsmUpsmU |
| 35 | 35 | Sense | GGACAUUUUUGGA | 35 | 350 | mGpsmGpsmAmCmAmUfUmU |
| | | | UCACAAAA | | | fUfUfGmGmAmUmCmAmCm |
| | | | | | | AmAmApsmA |
| | 135 | Anti- | UUUUGUGAUCCAA | | 480 | vmUpsfUpsmUmUmGfUmGmA |
| | | Sense | AAAUGUCCUU | | | mUmCmCmAmAfAmAfAmUm |
| | | | | | | GmUmCmCpsmUpsmU |
| 36 | 36 | Sense | UUUUUGGAUCACA | 36 | 351 | mUpsmUpsmUmUmUmGfGmA |
| | | | AAAGCACU | | | fUfCfAmCmAmAmAmAmGm |
| | | | | | | CmAmCpsmU |
| | 136 | Anti- | AGUGCUUUUGUGA | | 481 | vmApsfGpsmUmGmCfUmUmU |
| | | Sense | UCCAAAAAUU | | | mUmGmUmGmAfUmCfCmAm |
| | | | | | | AmAmAmApsmUpsmU |
| 37 | 37 | Sense | UUGGAUCACAAAA | 37 | 352 | mUpsmUpsmGmGmAmUfCmA |
| | | | GCACUUCU | | | fCfAfAmAmAmGmCmAmCm |
| | | | | | | UmUmCpsmU |
| | 137 | Anti- | AGAAGUGCUUUUG | | 482 | vmApsfGpsmAmAmGfUmGmC |
| | | Sense | UGAUCCAAUU | | | mUmUmUmUmGfUmGfAmUm |
| | | | | | | CmCmAmApsmUpsmU |
| 38 | 38 | Sense | AUCACAAAAGCAC | 38 | 353 | mApsmUpsmCmAmCmAfAmA |
| | | | UUCUUCCA | | | fAfGfCmAmCmUmUmCmUm |
| | | | | | | UmCmCpsmA |
| | 138 | Anti- | UGGAAGAAGUGCU | | 483 | vmUpsfGpsmGmAmAfGmAmA |
| | | Sense | UUUGUGAUUU | | | mGmUmGmCmUfUmUfUmGm |
| | | | | | | UmGmAmUpsmUpsmU |
| 39 | 39 | Sense | UCACAAAAGCACU | 39 | 354 | mUpsmCpsmAmCmAmAfAmA |
| | | | UCUUCCAU | | | fGfCfAmCmUmUmCmUmUmC |
| | | | | | | mCmApsmU |
| | 139 | Anti- | AUGGAAGAAGUGC | | 484 | vmApsfUpsmGmGmAfAmGmA |
| | | Sense | UUUUGUGAUU | | | mAmGmUmGmCfUmUfUmUm |
| | | | | | | GmUmGmApsmUpsmU |
| 40 | 40 | Sense | AAGCACUUCUUCC | 40 | 355 | mApsmApsmGmCmAmCfUmU |
| | | | AUCGAUGA | | | fCfUfUmCmCmAmUmCmGmA |
| | | | | | | mUmGpsmA |
| | 140 | Anti- | UCAUCGAUGGAAG | | 485 | vmUpsfCpsmAmUmCfGmAmU |
| | | Sense | AAGUGCUUUU | | | mGmGmAmAmGfAmAfGmUm |
| | | | | | | GmCmUmUpsmUpsmU |
| 41 | 41 | Sense | AGCACUUCUUCCA | 41 | 356 | mApsmGpsmCmAmCmUfUmC |
| | | | UCGAUGAU | | | fUfUfCmCmAmUmCmGmAm |
| | | | | | | UmGmApsmU |
| | 141 | Anti- | AUCAUCGAUGGAA | | 486 | vmApsfUpsmCmAmUfCmGmA |
| | | Sense | GAAGUGCUUU | | | mUmGmGmAmAfGmAfAmGm |
| | | | | | | UmGmCmUpsmUpsmU |
| 42 | 42 | Sense | UUCCAUCGAUGAU | 42 | 357 | mUpsmUpsmCmCmAmUfCmG |
| | | | GGAGAGAA | | | fAfUfGmAmUmGmGmAmGm |
| | | | | | | AmGmApsmA |
| | 142 | Anti- | UUCUCUCCAUCAU | | 487 | vmUpsfUpsmCmUmCfUmCmC |
| | | Sense | CGAUGGAAUU | | | mAmUmCmAmUfCmGfAmUm |
| | | | | | | GmGmAmApsmUpsmU |
| 43 | 43 | Sense | CAUCGUCACAGUG | 43 | 358 | mCpsmApsmUmCmGmUfCmA |
| | | | GCUUCAGU | | | fCfAfGmUmGmGmCmUmUm |
| | | | | | | CmAmGpsmU |
| | 143 | Anti- | ACUGAAGCCACUG | | 488 | vmApsfCpsmUmGmAfAmGmC |
| | | Sense | UGACGAUGUU | | | mCmAmCmUmGfUmGfAmCm |
| | | | | | | GmAmUmGpsmUpsmU |
| 44 | 44 | Sense | UCGUCACAGUGGC | 44 | 359 | mUpsmCpsmGmUmCmAfCmA |
| | | | UUCAGUGU | | | fGfUfGmGmCmUmUmCmAm |
| | | | | | | GmUmGpsmU |
| | 144 | Anti- | ACACUGAAGCCAC | | 489 | vmApsfCpsmAmCmUfGmAmA |
| | | Sense | UGUGACGAUU | | | mGmCmCmAmCfUmGfUmGm |
| | | | | | | AmCmGmApsmUpsmU |
| 45 | 45 | Sense | GAUUCCUUACCUC | 45 | 360 | mGpsmApsmUmUmCmCfUmU |
| | | | AUCCCAUA | | | fAfCfCmUmCmAmUmCmCmC |
| | | | | | | mAmUpsmA |
| | 145 | Anti- | UAUGGGAUGAGGU | | 490 | vmUpsfApsmUmGmGfGmAmU |
| | | Sense | AAGGAAUCUU | | | mGmAmGmGmUfAmAfGmGm |
| | | | | | | AmAmUmCpsmUpsmU |
| 46 | 46 | Sense | AUUCCUUACCUCA | 46 | 361 | mApsmUpsmUmCmCmUfUmA |
| | | | UCCCAUAU | | | fCfCfUmCmAmUmCmCmCmA |
| | | | | | | mUmApsmU |
| | 146 | Anti- | AUAUGGGAUGAGG | | 491 | vmApsfUpsmAmUmGfGmGmA |
| | | Sense | UAAGGAAUUU | | | mUmGmAmGmGfUmAfAmGm |
| | | | | | | GmAmAmUpsmUpsmU |
| 47 | 47 | Sense | UUCCUUACCUCAU | 47 | 362 | mUpsmUpsmCmCmUmUfAmC |
| | | | CCCAUAUU | | | fCfUfCmAmUmCmCmCmAmU |
| | | | | | | mAmUpsmU |
| | 147 | Anti- | AAUAUGGGAUGAG | | 492 | vmApsfApsmUmAmUfGmGmG |
| | | Sense | GUAAGGAAUU | | | mAmUmGmAmGfGmUfAmAm |
| | | | | | | GmGmAmApsmUpsmU |
| 48 | 48 | Sense | AUCCCAUAUUGUU | 48 | 363 | mApsmUpsmCmCmCmAfUmA |
| | | | CCAGCAAA | | | fUfUfGmUmUmCmCmAmGm |
| | | | | | | CmAmApsmA |
| | 148 | Anti- | UUUGCUGGAACAA | | 493 | vmUpsfUpsmUmGmCfUmGmG |
| | | Sense | UAUGGGAUUU | | | mAmAmCmAmAfUmAfUmGm |
| | | | | | | GmGmAmUpsmUpsmU |
| 49 | 49 | Sense | UCCCAUAUUGUUC | 49 | 364 | mUpsmCpsmCmCmAmUfAmU |
| | | | CAGCAAAU | | | fUfGfUmUmCmCmAmGmCm |
| | | | | | | AmAmApsmU |
| | 149 | Anti- | AUUUGCUGGAACA | | 494 | vmApsfUpsmUmUmGfCmUmG |
| | | Sense | AUAUGGGAUU | | | mGmAmAmCmAfAmUfAmUm |
| | | | | | | GmGmGmApsmUpsmU |
| 50 | 50 | Sense | UUGGGAAAAACUG | 50 | 365 | mUpsmUpsmGmGmGmAfAmA |
| | | | GUAUCAAA | | | fAfAfCmUmGmGmUmAmUm |
| | | | | | | CmAmApsmA |
| | 150 | Anti- | UUUGAUACCAGUU | | 495 | vmUpsfUpsmUmGmAfUmAmC |
| | | Sense | UUUCCCAAUU | | | mCmAmGmUmUfUmUfUmCm |
| | | | | | | CmCmAmApsmUpsmU |
| 51 | 51 | Sense | AAAACUGGUAUCA | 51 | 366 | mApsmApsmAmAmCmUfGmG |
| | | | AAACCUCA | | | fUfAfUmCmAmAmAmAmCm |
| | | | | | | CmUmCpsmA |
| | 151 | Anti- | UGAGGUUUUGAUA | | 496 | vmUpsfGpsmAmGmGfUmUmU |
| | | Sense | CCAGUUUUUU | | | mUmGmAmUmAfCmCfAmGm |
| | | | | | | UmUmUmUpsmUpsmU |
| 52 | 52 | Sense | AAUACUGGGUUCA | 52 | 367 | mApsmApsmUmAmCmUfGmG |
| | | | CCAAAAAU | | | fGfUfUmCmAmCmCmAmAm |
| | | | | | | AmAmApsmU |
| | 152 | Anti- | AUUUUUGGUGAAC | | 497 | vmApsfUpsmUmUmUfUmGmG |
| | | Sense | CCAGUAUUUU | | | mUmGmAmAmCfCmCfAmGm |
| | | | | | | UmAmUmUpsmUpsmU |
| 53 | 53 | Sense | CUGGGUUCACCAA | 53 | 368 | mCpsmUpsmGmGmGmUfUmC |
| | | | AAAUCCAA | | | fAfCfCmAmAmAmAmAmUm |
| | | | | | | CmCmApsmA |
| | 153 | Anti- | UUGGAUUUUUGGU | | 498 | vmUpsfUpsmGmGmAfUmUmU |
| | | Sense | GAACCCAGUU | | | mUmUmGmGmUfGmAfAmCm |
| | | | | | | CmCmAmGpsmUpsmU |
| 54 | 54 | Sense | ACCAAAAAUCCAA | 54 | 369 | mApsmCpsmCmAmAmAfAmA |
| | | | GCACAAGA | | | fUfCfCmAmAmGmCmAmCmA |
| | | | | | | mAmGpsmA |
| | 154 | Anti- | UCUUGUGCUUGGA | | 499 | vmUpsfCpsmUmUmGfUmGmC |
| | | Sense | UUUUUGGUUU | | | mUmUmGmGmAfUmUfUmUm |
| | | | | | | UmGmGmUpsmUpsmU |
| 55 | 55 | Sense | GCCUGUAUUGGAG | 55 | 370 | mGpsmCpsmCmUmGmUfAmU |
| | | | ACAGAUGA | | | fUfGfGmAmGmAmCmAmGm |
| | | | | | | AmUmGpsmA |
| | 155 | Anti- | UCAUCUGUCUCCA | | 500 | vmUpsfCpsmAmUmCfUmGmU |
| | | Sense | AUACAGGCUU | | | mCmUmCmCmAfAmUfAmCm |
| | | | | | | AmGmGmCpsmUpsmU |
| 56 | 56 | Sense | UGAAGUCGUAAGA | 56 | 371 | mUpsmGpsmAmAmGmUfCmG |
| | | | AGUCUGAU | | | fUfAfAmGmAmAmGmUmCm |
| | | | | | | UmGmApsmU |
| | 156 | Anti- | AUCAGACUUCUUA | | 501 | vmApsfUpsmCmAmGfAmCmU |
| | | Sense | CGACUUCAUU | | | mUmCmUmUmAfCmGfAmCm |
| | | | | | | UmUmCmApsmUpsmU |
| 57 | 57 | Sense | UAAGAAGUCUGAU | 57 | 372 | mUpsmApsmAmGmAmAfGmU |
| | | | AGAUGGAA | | | fCfUfGmAmUmAmGmAmUm |
| | | | | | | GmGmApsmA |
| | 157 | Anti- | UUCCAUCUAUCAG | | 502 | vmUpsfUpsmCmCmAfUmCmU |
| | | Sense | ACUUCUUAUU | | | mAmUmCmAmGfAmCfUmUm |
| | | | | | | CmUmUmApsmUpsmU |
| 58 | 58 | Sense | AAGAAGUCUGAUA | 58 | 373 | mApsmApsmGmAmAmGfUmC |
| | | | GAUGGAAU | | | fUfGfAmUmAmGmAmUmGm |
| | | | | | | GmAmApsmU |
| | 158 | Anti- | AUUCCAUCUAUCA | | 503 | vmApsfUpsmUmCmCfAmUmC |
| | | Sense | GACUUCUUUU | | | mUmAmUmCmAfGmAfCmUm |
| | | | | | | UmCmUmUpsmUpsmU |
| 59 | 59 | Sense | AGAAGUCUGAUAG | 59 | 374 | mApsmGpsmAmAmGmUfCmU |
| | | | AUGGAAUA | | | fGfAfUmAmGmAmUmGmGm |
| | | | | | | AmAmUpsmA |
| | 159 | Anti- | UAUUCCAUCUAUC | | 504 | vmUpsfApsmUmUmCfCmAmU |
| | | Sense | AGACUUCUUU | | | mCmUmAmUmCfAmGfAmCm |
| | | | | | | UmUmCmUpsmUpsmU |
| 60 | 60 | Sense | AAGUCUGAUAGAU | 60 | 375 | mApsmApsmGmUmCmUfGmA |
| | | | GGAAUACU | | | fUfAfGmAmUmGmGmAmAm |
| | | | | | | UmAmCpsmU |
| | 160 | Anti- | AGUAUUCCAUCUA | | 505 | vmApsfGpsmUmAmUfUmCmC |
| | | Sense | UCAGACUUUU | | | mAmUmCmUmAfUmCfAmGm |
| | | | | | | AmCmUmUpsmUpsmU |
| 61 | 61 | Sense | AGUCUGAUAGAUG | 61 | 376 | mApsmGpsmUmCmUmGfAmU |
| | | | GAAUACUU | | | fAfGfAmUmGmGmAmAmUm |
| | | | | | | AmCmUpsmU |
| | 161 | Anti- | AAGUAUUCCAUCU | | 506 | vmApsfApsmGmUmAfUmUmC |
| | | Sense | AUCAGACUUU | | | mCmAmUmCmUfAmUfCmAm |
| | | | | | | GmAmCmUpsmUpsmU |
| 62 | 62 | Sense | GUCUGAUAGAUGG | 62 | 377 | mGpsmUpsmCmUmGmAfUmA |
| | | | AAUACUUA | | | fGfAfUmGmGmAmAmUmAm |
| | | | | | | CmUmUpsmA |
| | 162 | Anti- | UAAGUAUUCCAUC | | 507 | vmUpsfApsmAmGmUfAmUmU |
| | | Sense | UAUCAGACUU | | | mCmCmAmUmCfUmAfUmCm |
| | | | | | | AmGmAmCpsmUpsmU |
| 63 | 63 | Sense | AGAUGGAAUACUU | 63 | 378 | mApsmGpsmAmUmGmGfAmA |
| | | | ACCAAUAA | | | fUfAfCmUmUmAmCmCmAm |
| | | | | | | AmUmApsmA |
| | 163 | Anti- | UUAUUGGUAAGUA | | 508 | vmUpsfUpsmAmUmUfGmGmU |
| | | Sense | UUCCAUCUUU | | | mAmAmGmUmAfUmUfCmCm |
| | | | | | | AmUmCmUpsmUpsmU |
| 64 | 64 | Sense | UGGAAUACUUACC | 64 | 379 | mUpsmGpsmGmAmAmUfAmC |
| | | | AAUAAGAA | | | fUfUfAmCmCmAmAmUmAm |
| | | | | | | AmGmApsmA |
| | 164 | Anti- | UUCUUAUUGGUAA | | 509 | vmUpsfUpsmCmUmUfAmUmU |
| | | Sense | GUAUUCCAUU | | | mGmGmUmAmAfGmUfAmUm |
| | | | | | | UmCmCmApsmUpsmU |
| 65 | 65 | Sense | GAAUACUUACCAA | 65 | 380 | mGpsmApsmAmUmAmCfUmU |
| | | | UAAGAAAA | | | fAfCfCmAmAmUmAmAmGm |
| | | | | | | AmAmApsmA |
| | 165 | Anti- | UUUUCUUAUUGGU | | 510 | vmUpsfUpsmUmUmCfUmUmA |
| | | Sense | AAGUAUUCUU | | | mUmUmGmGmUfAmAfGmUm |
| | | | | | | AmUmUmCpsmUpsmU |
| 66 | 66 | Sense | AAUACUUACCAAU | 66 | 381 | mApsmApsmUmAmCmUfUmA |
| | | | AAGAAAAU | | | fCfCfAmAmUmAmAmGmAm |
| | | | | | | AmAmApsmU |
| | 166 | Anti- | AUUUUCUUAUUGG | | 511 | vmApsfUpsmUmUmUfCmUmU |
| | | Sense | UAAGUAUUUU | | | mAmUmUmGmGfUmAfAmGm |
| | | | | | | UmAmUmUpsmUpsmU |
| 67 | 67 | Sense | ACUUACCAAUAAG | 67 | 382 | mApsmCpsmUmUmAmCfCmA |
| | | | AAAAUGAU | | | fAfUfAmAmGmAmAmAmAm |
| | | | | | | UmGmApsmU |
| | 167 | Anti- | AUCAUUUUCUUAU | | 512 | vmApsfUpsmCmAmUfUmUmU |
| | | Sense | UGGUAAGUUU | | | mCmUmUmAmUfUmGfGmUm |
| | | | | | | AmAmGmUpsmUpsmU |
| 68 | 68 | Sense | CUUACCAAUAAGA | 68 | 383 | mCpsmUpsmUmAmCmCfAmA |
| | | | AAAUGAUU | | | fUfAfAmGmAmAmAmAmUm |
| | | | | | | GmAmUpsmU |
| | 168 | Anti- | AAUCAUUUUCUUA | | 513 | vmApsfApsmUmCmAfUmUmU |
| | | Sense | UUGGUAAGUU | | | mUmCmUmUmAfUmUfGmGm |
| | | | | | | UmAmAmGpsmUpsmU |
| 69 | 69 | Sense | UUACCAAUAAGAA | 69 | 384 | mUpsmUpsmAmCmCmAfAmU |
| | | | AAUGAUUU | | | fAfAfGmAmAmAmAmUmGm |
| | | | | | | AmUmUpsmU |
| | 169 | Anti- | AAAUCAUUUUCUU | | 514 | vmApsfApsmAmUmCfAmUmU |
| | | Sense | AUUGGUAAUU | | | mUmUmCmUmUfAmUfUmGm |
| | | | | | | GmUmAmApsmUpsmU |
| 70 | 70 | Sense | UACCAAUAAGAAA | 70 | 385 | mUpsmApsmCmCmAmAfUmA |
| | | | AUGAUUUU | | | fAfGfAmAmAmAmUmGmAm |
| | | | | | | UmUmUpsmU |
| | 170 | Anti- | AAAAUCAUUUUCU | | 515 | vmApsfApsmAmAmUfCmAmU |
| | | Sense | UAUUGGUAUU | | | mUmUmUmCmUfUmAfUmUm |
| | | | | | | GmGmUmApsmUpsmU |
| 71 | 71 | Sense | ACCAAUAAGAAAA | 71 | 386 | mApsmCpsmCmAmAmUfAmA |
| | | | UGAUUUUU | | | fGfAfAmAmAmUmGmAmUm |
| | | | | | | UmUmUpsmU |
| | 171 | Anti- | AAAAAUCAUUUUC | | 516 | vmApsfApsmAmAmAfUmCmA |
| | | Sense | UUAUUGGUUU | | | mUmUmUmUmCfUmUfAmUm |
| | | | | | | UmGmGmUpsmUpsmU |
| 72 | 72 | Sense | CAAUAAGAAAAUG | 72 | 387 | mCpsmApsmAmUmAmAfGmA |
| | | | AUUUUUGU | | | fAfAfAmUmGmAmUmUmUm |
| | | | | | | UmUmGpsmU |
| | 172 | Anti- | ACAAAAAUCAUUU | | 517 | vmApsfCpsmAmAmAfAmAmU |
| | | Sense | UCUUAUUGUU | | | mCmAmUmUmUfUmCfUmUm |
| | | | | | | AmUmUmGpsmUpsmU |
| 73 | 73 | Sense | AAUAAGAAAAUGA | 73 | 388 | mApsmApsmUmAmAmGfAmA |
| | | | UUUUUGUU | | | fAfAfUmGmAmUmUmUmUm |
| | | | | | | UmGmUpsmU |
| | 173 | Anti- | AACAAAAAUCAUU | | 518 | vmApsfApsmCmAmAfAmAmA |
| | | Sense | UUCUUAUUUU | | | mUmCmAmUmUfUmUfCmUm |
| | | | | | | UmAmUmUpsmUpsmU |
| 74 | 74 | Sense | AAGAAAAUGAUUU | 74 | 389 | mApsmApsmGmAmAmAfAmU |
| | | | UUGUUCCA | | | fGfAfUmUmUmUmUmGmUm |
| | | | | | | UmCmCpsmA |
| | 174 | Anti- | UGGAACAAAAAUC | | 519 | vmUpsfGpsmGmAmAfCmAmA |
| | | Sense | AUUUUCUUUU | | | mAmAmAmUmCfAmUfUmUm |
| | | | | | | UmCmUmUpsmUpsmU |
| 75 | 75 | Sense | AGAAAAUGAUUUU | 75 | 390 | mApsmGpsmAmAmAmAfUmG |
| | | | UGUUCCAU | | | fAfUfUmUmUmUmGmUmUm |
| | | | | | | CmCmApsmU |
| | 175 | Anti- | AUGGAACAAAAAU | | 520 | vmApsfUpsmGmGmAfAmCmA |
| | | Sense | CAUUUUCUUU | | | mAmAmAmAmUfCmAfUmUm |
| | | | | | | UmUmCmUpsmUpsmU |
| 76 | 76 | Sense | AAUGAUUUUUGUU | 76 | 391 | mApsmApsmUmGmAmUfUmU |
| | | | CCAUCGUA | | | fUfUfGmUmUmCmCmAmUm |
| | | | | | | CmGmUpsmA |
| | 176 | Anti- | UACGAUGGAACAA | | 521 | vmUpsfApsmCmGmAfUmGmG |
| | | Sense | AAAUCAUUUU | | | mAmAmCmAmAfAmAfAmUm |
| | | | | | | CmAmUmUpsmUpsmU |
| 77 | 77 | Sense | AUGAUUUUUGUUC | 77 | 392 | mApsmUpsmGmAmUmUfUmU |
| | | | CAUCGUAU | | | fUfGfUmUmCmCmAmUmCm |
| | | | | | | GmUmApsmU |
| | 177 | Anti- | AUACGAUGGAACA | | 522 | vmApsfUpsmAmCmGfAmUmG |
| | | Sense | AAAAUCAUUU | | | mGmAmAmCmAfAmAfAmAm |
| | | | | | | UmCmAmUpsmUpsmU |
| 78 | 78 | Sense | UUUUGUUCCAUCG | 78 | 393 | mUpsmUpsmUmUmGmUfUmC |
| | | | UAUAUCAA | | | fCfAfUmCmGmUmAmUmAm |
| | | | | | | UmCmApsmA |
| | 178 | Anti- | UUGAUAUACGAUG | | 523 | vmUpsfUpsmGmAmUfAmUmA |
| | | Sense | GAACAAAAUU | | | mCmGmAmUmGfGmAfAmCm |
| | | | | | | AmAmAmApsmUpsmU |
| 79 | 79 | Sense | UUUGUUCCAUCGU | 79 | 394 | mUpsmUpsmUmGmUmUfCmC |
| | | | AUAUCAAU | | | fAfUfCmGmUmAmUmAmUm |
| | | | | | | CmAmApsmU |
| | 179 | Anti- | AUUGAUAUACGAU | | 524 | vmApsfUpsmUmGmAfUmAmU |
| | | Sense | GGAACAAAUU | | | mAmCmGmAmUfGmGfAmAm |
| | | | | | | CmAmAmApsmUpsmU |
| 80 | 80 | Sense | UUCAUUCCUCAGA | 80 | 395 | mUpsmUpsmCmAmUmUfCmC |
| | | | GGAGAAAA | | | fUfCfAmGmAmGmGmAmGm |
| | | | | | | AmAmApsmA |
| | 180 | Anti- | UUUUCUCCUCUGA | | 525 | vmUpsfUpsmUmUmCfUmCmC |
| | | Sense | GGAAUGAAUU | | | mUmCmUmGmAfGmGfAmAm |
| | | | | | | UmGmAmApsmUpsmU |
| 81 | 81 | Sense | AGAAAGAAGUGGG | 81 | 396 | mApsmGpsmAmAmAmGfAmA |
| | | | UGAUGUAA | | | fGfUfGmGmGmUmGmAmUm |
| | | | | | | GmUmApsmA |
| | 181 | Anti- | UUACAUCACCCAC | | 526 | vmUpsfUpsmAmCmAfUmCmA |
| | | Sense | UUCUUUCUUU | | | mCmCmCmAmCfUmUfCmUm |
| | | | | | | UmUmCmUpsmUpsmU |
| 82 | 82 | Sense | CAAGGAUGAAGAG | 82 | 397 | mCpsmApsmAmGmGmAfUmG |
| | | | AUUACCAA | | | fAfAfGmAmGmAmUmUmAm |
| | | | | | | CmCmApsmA |
| | 182 | Anti- | UUGGUAAUCUCUU | | 527 | vmUpsfUpsmGmGmUfAmAmU |
| | | Sense | CAUCCUUGUU | | | mCmUmCmUmUfCmAfUmCm |
| | | | | | | CmUmUmGpsmUpsmU |
| 83 | 83 | Sense | CUAGGACAUUUUU | 83 | 398 | mCpsmUpsmAmGmGmAfCmA |
| | | | GGAUCACA | | | fUfUfUmUmUmGmGmAmUm |
| | | | | | | CmAmCpsmA |
| | 183 | Anti- | UGUGAUCCAAAAA | | 528 | vmUpsfGpsmUmGmAfUmCmC |
| | | Sense | UGUCCUAGUU | | | mAmAmAmAmAfUmGfUmCm |
| | | | | | | CmUmAmGpsmUpsmU |
| 84 | 84 | Sense | UGGAUCACAAAAG | 84 | 399 | mUpsmGpsmGmAmUmCfAmC |
| | | | CACUUCUU | | | fAfAfAmAmGmCmAmCmUm |
| | | | | | | UmCmUpsmU |
| | 184 | Anti- | AAGAAGUGCUUUU | | 529 | vmApsfApsmGmAmAfGmUmG |
| | | Sense | GUGAUCCAUU | | | mCmUmUmUmUfGmUfGmAm |
| | | | | | | UmCmCmApsmUpsmU |

mX = 2′-O-methyl nucleotide; fX = 2′-fluoro nucleotide; v = 5′ vinyl phosphonate; vmX = 5′ vinyl phosphonate 2′-O-methyl nucleotide; ps = phosphorothioate linkage.

TABLE 9
21-mers with 3′-GalNAc in S and 5′-VP in AS

| | SEQ | Sense/ | | | SEQ | |
|---|---|---|---|---|---|---|
| | ID | Anti- | | | ID | |
| D# | NO. | sense | Base Sequence (5′-3′) | MD# | NO. | Modified Sequence (5′-3′) |
| 85 | 85 | Sense | UGGAGUCGUUGGU | 85 | 400 | mUpsmGpsmGmAmGmUfCmG |
| | | | GAAGUUUU | | | fUfUfGmGmUmGmAmAmGm |
| | | | | | | UmUmUpsmU-p-ps2-GalNAc4 |
| | 185 | Anti- | AAAACUUCACCAA | | 530 | vmApsfApsmAmAmCfUmUmC |
| | | sense | CGACUCCAUU | | | mAmCmCmAmAfCmGfAmCm |
| | | | | | | UmCmCmApsmUpsmU |
| 86 | 86 | Sense | UAUUGGUUCUGUG | 86 | 401 | mUpsmApsmUmUmGmGfUmU |
| | | | GGAUAUUA | | | fCfUfGmUmGmGmGmAmUm |
| | | | | | | AmUmUpsmA-p-ps2-GalNAc4 |
| | 186 | Anti- | UAAUAUCCCACAG | | 531 | vmUpsfApsmAmUmAfUmCmC |
| | | sense | AACCAAUAUU | | | mCmAmCmAmGfAmAfCmCm |
| | | | | | | AmAmUmApsmUpsmU |
| 87 | 87 | Sense | CUAGGACAUUUUU | 87 | 402 | mCpsmUpsmAmGmGmAfCmA |
| | | | GGAUCACA | | | fUfUfUmUmUmGmGmAmUm |
| | | | | | | CmAmCpsmA-p-ps2-GalNAc4 |
| | 187 | Anti- | UGUGAUCCAAAAA | | 532 | vmUpsfGpsmUmGmAfUmCmC |
| | | sense | UGUCCUAGUU | | | mAmAmAmAmAfUmGfUmCm |
| | | | | | | CmUmAmGpsmUpsmU |
| 88 | 88 | Sense | UUGGGAAAAACUG | 88 | 403 | mUpsmUpsmGmGmGmAfAmA |
| | | | GUAUCAAA | | | fAfAfCmUmGmGmUmAmUm |
| | | | | | | CmAmApsmA-p-ps2-GalNAc4 |
| | 188 | Anti- | UUUGAUACCAGUU | | 533 | vmUpsfUpsmUmGmAfUmAmC |
| | | sense | UUUCCCAAUU | | | mCmAmGmUmUfUmUfUmCm |
| | | | | | | CmCmAmApsmUpsmU |

mX = 2′-O-methyl nucleotide; fX = 2′-fluoro nucleotide; v = 5′ vinyl phosphonate; vmX = 5′ vinyl phosphonate 2′-O-methyl nucleotide; ps = phosphorothioate linkage.

TABLE 10
21-mers with 5′-VP in AS (continued from Table 4)

| | SEQ | Sense/ | | | SEQ | |
|---|---|---|---|---|---|---|
| | ID | Anti- | Base Sequence | | ID | |
| D# | NO. | sense | (5′-3′) | MD# | NO. | Modified Sequence (5′-3′) |
| 89 | 89 | Sense | CCUUACCUCAUC | 89 | 404 | mCpsmCpsmUmUmAmCfCm |
| | | | CCAUAUUGU | | | UfCfAfUmCmCmCmAmUmA |
| | | | | | | mUmUmGpsmU |
| | 189 | Anti- | ACAAUAUGGGAU | | 534 | vmApsfCpsmAmAmUfAmUm |
| | | Sense | GAGGUAAGGUU | | | GmGmGmAmUmGfAmGfGm |
| | | | | | | UmAmAmGmGpsmUpsmU |
| 90 | 90 | Sense | CCCAUAUUGUUC | 90 | 405 | mCpsmCpsmCmAmUmAfUm |
| | | | CAGCAAAUU | | | UfGfUfUmCmCmAmGmCmA |
| | | | | | | mAmAmUpsmU |
| | 190 | Anti- | AAUUUGCUGGAA | | 535 | vmApsfApsmUmUmUfGmCm |
| | | Sense | CAAUAUGGGUU | | | UmGmGmAmAmCfAmAfUm |
| | | | | | | AmUmGmGmGpsmUpsmU |
| 91 | 91 | Sense | CCUUGGGAAAAA | 91 | 406 | mCpsmCpsmUmUmGmGfGm |
| | | | CUGGUAUCA | | | AfAfAfAmAmCmUmGmGmU |
| | | | | | | mAmUmCpsmA |
| | 191 | Anti- | UGAUACCAGUUU | | 536 | vmUpsfGpsmAmUmAfCmCm |
| | | Sense | UUCCCAAGGUU | | | AmGmUmUmUmUfUmCfCm |
| | | | | | | CmAmAmGmGpsmUpsmU |
| 92 | 92 | Sense | UGGUAUCAAAAC | 92 | 407 | mUpsmGpsmGmUmAmUfCm |
| | | | CUCAUGUCU | | | AfAfAfAmCmCmUmCmAmU |
| | | | | | | mGmUmCpsmU |
| | 192 | Anti- | AGACAUGAGGUU | | 537 | vmApsfGpsmAmCmAfUmGm |
| | | Sense | UUGAUACCAUU | | | AmGmGmUmUmUfUmGfAm |
| | | | | | | UmAmCmCmApsmUpsmU |
| 93 | 93 | Sense | GUAUCAAAACCU | 93 | 408 | mGpsmUpsmAmUmCmAfAm |
| | | | CAUGUCUCU | | | AfAfCfCmUmCmAmUmGmU |
| | | | | | | mCmUmCpsmU |
| | 193 | Anti- | AGAGACAUGAGG | | 538 | vmApsfGpsmAmGmAfCmAm |
| | | Sense | UUUUGAUACUU | | | UmGmAmGmGmUfUmUfUm |
| | | | | | | GmAmUmAmCpsmUpsmU |
| 94 | 94 | Sense | UUGUGAAUACUG | 94 | 409 | mUpsmUpsmGmUmGmAfAm |
| | | | GGUUCACCA | | | UfAfCfUmGmGmGmUmUmC |
| | | | | | | mAmCmCpsmA |
| | 194 | Anti- | UGGUGAACCCAG | | 539 | vmUpsfGpsmGmUmGfAmAm |
| | | Sense | UAUUCACAAUU | | | CmCmCmAmGmUfAmUfUm |
| | | | | | | CmAmCmAmApsmUpsmU |
| 95 | 95 | Sense | UGUGAAUACUGG | 95 | 410 | mUpsmGpsmUmGmAmAfUm |
| | | | GUUCACCAA | | | AfCfUfGmGmGmUmUmCmA |
| | | | | | | mCmCmApsmA |
| | 195 | Anti- | UUGGUGAACCCA | | 540 | vmUpsfUpsmGmGmUfGmAm |
| | | Sense | GUAUUCACAUU | | | AmCmCmCmAmGfUmAfUm |
| | | | | | | UmCmAmCmApsmUpsmU |
| 96 | 96 | Sense | GUGAAUACUGGG | 96 | 411 | mGpsmUpsmGmAmAmUfAm |
| | | | UUCACCAAA | | | CfUfGfGmGmUmUmCmAmC |
| | | | | | | mCmAmApsmA |
| | 196 | Anti- | UUUGGUGAACCC | | 541 | vmUpsfUpsmUmGmGfUmGm |
| | | Sense | AGUAUUCACUU | | | AmAmCmCmCmAfGmUfAm |
| | | | | | | UmUmCmAmCpsmUpsmU |
| 97 | 97 | Sense | UGAAUACUGGGU | 97 | 412 | mUpsmGpsmAmAmUmAfCm |
| | | | UCACCAAAA | | | UfGfGfGmUmUmCmAmCmC |
| | | | | | | mAmAmApsmA |
| | 197 | Anti- | UUUUGGUGAACC | | 542 | vmUpsfUpsmUmUmGfGmUm |
| | | Sense | CAGUAUUCAUU | | | GmAmAmCmCmCfAmGfUm |
| | | | | | | AmUmUmCmApsmUpsmU |
| 98 | 98 | Sense | CCAAAAAUCCAA | 98 | 413 | mCpsmCpsmAmAmAmAfAm |
| | | | GCACAAGAU | | | UfCfCfAmAmGmCmAmCmA |
| | | | | | | mAmGmApsmU |
| | 198 | Anti- | AUCUUGUGCUUG | | 543 | vmApsfUpsmCmUmUfGmUm |
| | | Sense | GAUUUUUGGUU | | | GmCmUmUmGmGfAmUfUm |
| | | | | | | UmUmUmGmGpsmUpsmU |
| 99 | 99 | Sense | GGAAUACUUACC | 99 | 414 | mGpsmGpsmAmAmUmAfCm |
| | | | AAUAAGAAA | | | UfUfAfCmCmAmAmUmAmA |
| | | | | | | mGmAmApsmA |
| | 199 | Anti- | UUUCUUAUUGGU | | 544 | vmUpsfUpsmUmCmUfUmAm |
| | | Sense | AAGUAUUCCUU | | | UmUmGmGmUmAfAmGfUm |
| | | | | | | AmUmUmCmCpsmUpsmU |
| 100 | 100 | Sense | UAGGACAUUUUU | 100 | 415 | mUpsmApsmGmGmAmCfAm |
| | | | GGAUCACAA | | | UfUfUfUmUmGmGmAmUmC |
| | | | | | | mAmCmApsmA |
| | 200 | Anti- | UUGUGAUCCAAA | | 545 | vmUpsfUpsmGmUmGfAmUm |
| | | Sense | AAUGUCCUAUU | | | CmCmAmAmAmAfAmUfGm |
| | | | | | | UmCmCmUmApsmUpsmU |

mX = 2′-O-methyl nucleotide; fX = 2′-fluoro nucleotide; v = 5′ vinyl phosphonate; vmX = 5′ vinyl phosphonate 2′-O-methyl nucleotide; ps = phosphorothioate linkage.

TABLE 11
21-mers without 5′-VP

| | SEQ | Sense/ | | | SEQ | |
|---|---|---|---|---|---|---|
| | ID | Anti- | Base Sequence | | ID | |
| D# | NO. | sense | (5′-3′) | MD# | NO. | Modified Sequence (5′-3′) |
| 101 | 201 | Sense | CUUGGAGUCGUUG | 101 | 416 | mCpsmUpsmUmGmGmAfGmU |
| | | | GUGAAGUU | | | fCfGfUmUmGmGmUmGmAm |
| | | | | | | AmGmUpsmU |
| | 231 | Anti- | AACUUCACCAACG | | 546 | mApsfApsmCmUmUfCmAmC |
| | | Sense | ACUCCAAGUU | | | mCmAmAmCmGfAmCfUmCm |
| | | | | | | CmAmAmGpsmUpsmU |
| 102 | 202 | Sense | UGGAGUCGUUGGU | 102 | 417 | mUpsmGpsmGmAmGmUfCmG |
| | | | GAAGUUUU | | | fUfUfGmGmUmGmAmAmGm |
| | | | | | | UmUmUpsmU |
| | 232 | Anti- | AAAACUUCACCAA | | 547 | mApsfApsmAmAmCfUmUmC |
| | | Sense | CGACUCCAUU | | | mAmCmCmAmAfCmGfAmCm |
| | | | | | | UmCmCmApsmUpsmU |
| 103 | 203 | Sense | UUGGUGAAGUUUU | 103 | 418 | mUpsmUpsmGmGmUmGfAmA |
| | | | UCAUUCCU | | | fGfUfUmUmUmUmCmAmUm |
| | | | | | | UmCmCpsmU |
| | 233 | Anti- | AGGAAUGAAAAAC | | 548 | mApsfGpsmGmAmAfUmGmA |
| | | Sense | UUCACCAAUU | | | mAmAmAmAmCfUmUfCmAm |
| | | | | | | CmCmAmApsmUpsmU |
| 104 | 204 | Sense | GGUGAAGUUUUUC | 104 | 419 | mGpsmGpsmUmGmAmAfGmU |
| | | | AUUCCUCA | | | fUfUfUmUmCmAmUmUmCm |
| | | | | | | CmUmCpsmA |
| | 234 | Anti- | UGAGGAAUGAAAA | | 549 | mUpsfGpsmAmGmGfAmAmU |
| | | Sense | ACUUCACCUU | | | mGmAmAmAmAfAmCfUmUm |
| | | | | | | CmAmCmCpsmUpsmU |
| 105 | 205 | Sense | GGCAGGCAGACUA | 105 | 420 | mGpsmGpsmCmAmGmGfCmA |
| | | | CUUAUGAA | | | fGfAfCmUmAmCmUmUmAm |
| | | | | | | UmGmApsmA |
| | 235 | Anti- | UUCAUAAGUAGUC | | 550 | mUpsfUpsmCmAmUfAmAmG |
| | | Sense | UGCCUGCCUU | | | mUmAmGmUmCfUmGfCmCm |
| | | | | | | UmGmCmCpsmUpsmU |
| 106 | 206 | Sense | UAUUGGUUCUGUG | 106 | 421 | mUpsmApsmUmUmGmGfUmU |
| | | | GGAUAUUA | | | fCfUfGmUmGmGmGmAmUm |
| | | | | | | AmUmUpsmA |
| | 236 | Anti- | UAAUAUCCCACAG | | 551 | mUpsfApsmAmUmAfUmCmC |
| | | Sense | AACCAAUAUU | | | mCmAmCmAmGfAmAfCmCm |
| | | | | | | AmAmUmApsmUpsmU |
| 107 | 207 | Sense | GGUUCUGUGGGAU | 107 | 422 | mGpsmGpsmUmUmCmUfGmU |
| | | | AUUAAUAA | | | fGfGfGmAmUmAmUmUmAm |
| | | | | | | AmUmApsmA |
| | 237 | Anti- | UUAUUAAUAUCCC | | 552 | mUpsfUpsmAmUmUfAmAmU |
| | | Sense | ACAGAACCUU | | | mAmUmCmCmCfAmCfAmGm |
| | | | | | | AmAmCmCpsmUpsmU |
| 108 | 208 | Sense | CCAAGGAUGAAGA | 108 | 423 | mCpsmCpsmAmAmGmGfAmU |
| | | | GAUUACCA | | | fGfAfAmGmAmGmAmUmUm |
| | | | | | | AmCmCpsmA |
| | 238 | Anti- | UGGUAAUCUCUUC | | 553 | mUpsfGpsmGmUmAfAmUmC |
| | | Sense | AUCCUUGGUU | | | mUmCmUmUmCfAmUfCmCm |
| | | | | | | UmUmGmGpsmUpsmU |
| 109 | 209 | Sense | GAAGAGAUUACCA | 109 | 424 | mGpsmApsmAmGmAmGfAmU |
| | | | AGACAUUU | | | fUfAfCmCmAmAmGmAmCm |
| | | | | | | AmUmUpsmU |
| | 239 | Anti- | AAAUGUCUUGGUA | | 554 | mApsfApsmAmUmGfUmCmU |
| | | Sense | AUCUCUUCUU | | | mUmGmGmUmAfAmUfCmUm |
| | | | | | | CmUmUmCpsmUpsmU |
| 110 | 210 | Sense | AGAGAUUACCAAG | 110 | 425 | mApsmGpsmAmGmAmUfUmA |
| | | | ACAUUUGA | | | fCfCfAmAmGmAmCmAmUm |
| | | | | | | UmUmGpsmA |
| | 240 | Anti- | UCAAAUGUCUUGG | | 555 | mUpsfCpsmAmAmAfUmGmU |
| | | Sense | UAAUCUCUUU | | | mCmUmUmGmGfUmAfAmUm |
| | | | | | | CmUmCmUpsmUpsmU |
| 111 | 211 | Sense | GAUUACCAAGACA | 111 | 426 | mGpsmApsmUmUmAmCfCmA |
| | | | UUUGAGGU | | | fAfGfAmCmAmUmUmUmGm |
| | | | | | | AmGmGpsmU |
| | 241 | Anti- | ACCUCAAAUGUCU | | 556 | mApsfCpsmCmUmCfAmAmA |
| | | Sense | UGGUAAUCUU | | | mUmGmUmCmUfUmGfGmUm |
| | | | | | | AmAmUmCpsmUpsmU |
| 112 | 212 | Sense | UACCAAGACAUUU | 112 | 427 | mUpsmApsmCmCmAmAfGmA |
| | | | GAGGUCAA | | | fCfAfUmUmUmGmAmGmGm |
| | | | | | | UmCmApsmA |
| | 242 | Anti- | UUGACCUCAAAUG | | 557 | mUpsfUpsmGmAmCfCmUmC |
| | | Sense | UCUUGGUAUU | | | mAmAmAmUmGfUmCfUmUm |
| | | | | | | GmGmUmApsmUpsmU |
| 113 | 213 | Sense | UCCUAGGACAUUU | 113 | 428 | mUpsmCpsmCmUmAmGfGmA |
| | | | UUGGAUCA | | | fCfAfUmUmUmUmUmGmGm |
| | | | | | | AmUmCpsmA |
| | 243 | Anti- | UGAUCCAAAAAUG | | 558 | mUpsfGpsmAmUmCfCmAmA |
| | | Sense | UCCUAGGAUU | | | mAmAmAmUmGfUmCfCmUm |
| | | | | | | AmGmGmApsmUpsmU |
| 114 | 214 | Sense | CUAGGACAUUUUU | 114 | 429 | mCpsmUpsmAmGmGmAfCmA |
| | | | GGAUCACA | | | fUfUfUmUmUmGmGmAmUm |
| | | | | | | CmAmCpsmA |
| | 244 | Anti- | UGUGAUCCAAAAA | | 559 | mUpsfGpsmUmGmAfUmCmC |
| | | Sense | UGUCCUAGUU | | | mAmAmAmAmAfUmGfUmCm |
| | | | | | | CmUmAmGpsmUpsmU |
| 115 | 215 | Sense | UUUUUGGAUCACA | 115 | 430 | mUpsmUpsmUmUmUmGfGmA |
| | | | AAAGCACU | | | fUfCfAmCmAmAmAmAmGm |
| | | | | | | CmAmCpsmU |
| | 245 | Anti- | AGUGCUUUUGUGA | | 560 | mApsfGpsmUmGmCfUmUmU |
| | | Sense | UCCAAAAAUU | | | mUmGmUmGmAfUmCfCmAm |
| | | | | | | AmAmAmApsmUpsmU |
| 116 | 216 | Sense | UGGAUCACAAAAG | 116 | 431 | mUpsmGpsmGmAmUmCfAmC |
| | | | CACUUCUU | | | fAfAfAmAmGmCmAmCmUm |
| | | | | | | UmCmUpsmU |
| | 246 | Anti- | AAGAAGUGCUUUU | | 561 | mApsfApsmGmAmAfGmUmG |
| | | Sense | GUGAUCCAUU | | | mCmUmUmUmUfGmUfGmAm |
| | | | | | | UmCmCmApsmUpsmU |
| 117 | 217 | Sense | UUGGGAAAAACUG | 117 | 432 | mUpsmUpsmGmGmGmAfAmA |
| | | | GUAUCAAA | | | fAfAfCmUmGmGmUmAmUm |
| | | | | | | CmAmApsmA |
| | 247 | Anti- | UUUGAUACCAGUU | | 562 | mUpsfUpsmUmGmAfUmAmC |
| | | Sense | UUUCCCAAUU | | | mCmAmGmUmUfUmUfUmCm |
| | | | | | | CmCmAmApsmUpsmU |
| 118 | 218 | Sense | AAAACUGGUAUCA | 118 | 433 | mApsmApsmAmAmCmUfGmG |
| | | | AAACCUCA | | | fUfAfUmCmAmAmAmAmCm |
| | | | | | | CmUmCpsmA |
| | 248 | Anti- | UGAGGUUUUGAUA | | 563 | mUpsfGpsmAmGmGfUmUmU |
| | | Sense | CCAGUUUUUU | | | mUmGmAmUmAfCmCfAmGm |
| | | | | | | UmUmUmUpsmUpsmU |
| 119 | 219 | Sense | UGAAGUCGUAAGA | 119 | 434 | mUpsmGpsmAmAmGmUfCmG |
| | | | AGUCUGAU | | | fUfAfAmGmAmAmGmUmCm |
| | | | | | | UmGmApsmU |
| | 249 | Anti- | AUCAGACUUCUUA | | 564 | mApsfUpsmCmAmGfAmCmU |
| | | Sense | CGACUUCAUU | | | mUmCmUmUmAfCmGfAmCm |
| | | | | | | UmUmCmApsmUpsmU |
| 120 | 220 | Sense | AGUCUGAUAGAUG | 120 | 435 | mApsmGpsmUmCmUmGfAmU |
| | | | GAAUACUU | | | fAfGfAmUmGmGmAmAmUm |
| | | | | | | AmCmUpsmU |
| | 250 | Anti- | AAGUAUUCCAUCU | | 565 | mApsfApsmGmUmAfUmUmC |
| | | Sense | AUCAGACUUU | | | mCmAmUmCmUfAmUfCmAm |
| | | | | | | GmAmCmUpsmUpsmU |
| 121 | 221 | Sense | GUCUGAUAGAUGG | 121 | 436 | mGpsmUpsmCmUmGmAfUmA |
| | | | AAUACUUA | | | fGfAfUmGmGmAmAmUmAm |
| | | | | | | CmUmUpsmA |
| | 251 | Anti- | UAAGUAUUCCAUC | | 566 | mUpsfApsmAmGmUfAmUmU |
| | | Sense | UAUCAGACUU | | | mCmCmAmUmCfUmAfUmCm |
| | | | | | | AmGmAmCpsmUpsmU |
| 122 | 222 | Sense | AGAUGGAAUACUU | 122 | 437 | mApsmGpsmAmUmGmGfAmA |
| | | | ACCAAUAA | | | fUfAfCmUmUmAmCmCmAm |
| | | | | | | AmUmApsmA |
| | 252 | Anti- | UUAUUGGUAAGUA | | 567 | mUpsfUpsmAmUmUfGmGmU |
| | | Sense | UUCCAUCUUU | | | mAmAmGmUmAfUmUfCmCm |
| | | | | | | AmUmCmUpsmUpsmU |
| 123 | 223 | Sense | UGGAAUACUUACC | 123 | 438 | mUpsmGpsmGmAmAmUfAmC |
| | | | AAUAAGAA | | | fUfUfAmCmCmAmAmUmAm |
| | | | | | | AmGmApsmA |
| | 253 | Anti- | UUCUUAUUGGUAA | | 568 | mUpsfUpsmCmUmUfAmUmU |
| | | Sense | GUAUUCCAUU | | | mGmGmUmAmAfGmUfAmUm |
| | | | | | | UmCmCmApsmUpsmU |
| 124 | 224 | Sense | ACUUACCAAUAAG | 124 | 439 | mApsmCpsmUmUmAmCfCmA |
| | | | AAAAUGAU | | | fAfUfAmAmGmAmAmAmAm |
| | | | | | | UmGmApsmU |
| | 254 | Anti- | AUCAUUUUCUUAU | | 569 | mApsfUpsmCmAmUfUmUmU |
| | | Sense | UGGUAAGUUU | | | mCmUmUmAmUfUmGfGmUm |
| | | | | | | AmAmGmUpsmUpsmU |
| 125 | 225 | Sense | CUUACCAAUAAGA | 125 | 440 | mCpsmUpsmUmAmCmCfAmA |
| | | | AAAUGAUU | | | fUfAfAmGmAmAmAmAmUm |
| | | | | | | GmAmUpsmU |
| | 255 | Anti- | AAUCAUUUUCUUA | | 570 | mApsfApsmUmCmAfUmUmU |
| | | Sense | UUGGUAAGUU | | | mUmCmUmUmAfUmUfGmGm |
| | | | | | | UmAmAmGpsmUpsmU |
| 126 | 226 | Sense | ACCAAUAAGAAAA | 126 | 441 | mApsmCpsmCmAmAmUfAmA |
| | | | UGAUUUUU | | | fGfAfAmAmAmUmGmAmUm |
| | | | | | | UmUmUpsmU |
| | 256 | Anti- | AAAAAUCAUUUUC | | 571 | mApsfApsmAmAmAfUmCmA |
| | | Sense | UUAUUGGUUU | | | mUmUmUmUmCfUmUfAmUm |
| | | | | | | UmGmGmUpsmUpsmU |
| 127 | 227 | Sense | AAGAAAAUGAUUU | 127 | 442 | mApsmApsmGmAmAmAfAmU |
| | | | UUGUUCCA | | | fGfAfUmUmUmUmUmGmUm |
| | | | | | | UmCmCpsmA |
| | 257 | Anti- | UGGAACAAAAAUC | | 572 | mUpsfGpsmGmAmAfCmAmA |
| | | Sense | AUUUUCUUUU | | | mAmAmAmUmCfAmUfUmUm |
| | | | | | | UmCmUmUpsmUpsmU |
| 128 | 228 | Sense | AAUGAUUUUUGUU | 128 | 443 | mApsmApsmUmGmAmUfUmU |
| | | | CCAUCGUA | | | fUfUfGmUmUmCmCmAmUm |
| | | | | | | CmGmUpsmA |
| | 258 | Anti- | UACGAUGGAACAA | | 573 | mUpsfApsmCmGmAfUmGmG |
| | | Sense | AAAUCAUUUU | | | mAmAmCmAmAfAmAfAmUm |
| | | | | | | CmAmUmUpsmUpsmU |
| 129 | 229 | Sense | AUGAUUUUUGUUC | 129 | 444 | mApsmUpsmGmAmUmUfUmU |
| | | | CAUCGUAU | | | fUfGfUmUmCmCmAmUmCm |
| | | | | | | GmUmApsmU |
| | 259 | Anti- | AUACGAUGGAACA | | 574 | mApsfUpsmAmCmGfAmUmG |
| | | Sense | AAAAUCAUUU | | | mGmAmAmCmAfAmAfAmAm |
| | | | | | | UmCmAmUpsmUpsmU |
| 130 | 230 | Sense | UUUUGUUCCAUCG | 130 | 445 | mUpsmUpsmUmUmGmUfUmC |
| | | | UAUAUCAA | | | fCfAfUmCmGmUmAmUmAm |
| | | | | | | UmCmApsmA |
| | 260 | Anti- | UUGAUAUACGAUG | | 575 | mUpsfUpsmGmAmUfAmUmA |
| | | Sense | GAACAAAAUU | | | mCmGmAmUmGfGmAfAmCm |
| | | | | | | AmAmAmApsmUpsmU |

mX = 2′-O-methyl nucleotide; fX = 2′-fluoro nucleotide; ps = phosphorothioate linkage.

TABLE 12
GalNAc conjugated siRNAs

| | SEQ | Sense/ | | | SEQ | |
|---|---|---|---|---|---|---|
| | ID | Anti- | Base Sequence | | ID | |
| D# | NO. | sense | (5′-3′) | MD# | NO. | Modified Sequence (5′-3′) |
| 131 | 262 | Sense | CUUGGAGUCGUUG | 131 | 576 | mCpsmUpsmUmGmGmAfGmU |
| | | | GUGAAGUU | | | fCfGfUmUmGmGmUmGmAm |
| | | | | | | AmGmUpsmU- |
| | | | | | | GalNAc4psGalNAc4psGalNAc4 |
| | 288 | Anti- | AACUUCACCAACG | | 604 | mApsfApsmCmUmUfCmAmC |
| | | Sense | ACUCCAAGUU | | | mCmAmAmCmGfAmCfUmCm |
| | | | | | | CmAmAmGpsmUpsmU |
| 132 | 263 | Sense | UGGAGUCGUUGGU | 132 | 577 | mUpsmGpsmGmAfGmUfCfGfU |
| | | | GAAGUU | | | mUmGmGmUmGmAmAmGm |
| | | | | | | UmU- |
| | | | | | | GalNAc4psGalNAc4psGalNAc4 |
| | 289 | Anti- | AACUUCACCAACG | | 605 | vmApsfApsmCmUmUfCmAmC |
| | | Sense | ACUCCAAG | | | mCmAmAmCmGfAmCfUmCm |
| | | | | | | CmApsmApsmG |
| 133 | 264 | Sense | UGGAGUCGUUGGU | 133 | 578 | mUpsmGpsmGmAfGmUfCfGfU |
| | | | GAAGUU | | | mUmGmGmUmGmAmAmGm |
| | | | | | | UmU- |
| | | | | | | GalNAc4psGalNAc4psGalNAc4 |
| | 290 | Anti- | AACUUCACCAACG | | 606 | mApsfApsmCmUmUfCmAmC |
| | | Sense | ACUCCAAG | | | mCmAmAmCmGfAmCfUmCm |
| | | | | | | CmApsmApsmG |
| 134 | 265 | Sense | CUUGGAGUCGUUG | 134 | 579 | mCpsmUpsmUmGmGmAfGmU |
| | | | GUGAAGUU | | | fCfGfUmUmGmGmUmGmAm |
| | | | | | | AmGmUmU- |
| | | | | | | GalNAc4psGalNAc4psGalNAc4 |
| | 291 | Anti- | AACUUCACCAACG | | 607 | vmApsfApsmCmUmUfCmAmC |
| | | Sense | ACUCCAAGUA | | | mCmAmAmCmGfAmCfUmCm |
| | | | | | | CmAmAmGpsmUpsmA |
| 135 | 266 | Sense | CUUGGAGUCGUUG | 135 | 580 | mCpsmUpsmUmGmGmAfGmU |
| | | | GUGAAGUU | | | fCfGfUmUmGmGmUmGmAm |
| | | | | | | AmGmUmU- |
| | | | | | | GalNAc4psGalNAc4psGalNAc4 |
| | 292 | Anti- | AACUUCACCAACG | | 608 | mApsfApsmCmUmUfCmAmC |
| | | Sense | ACUCCAAGUA | | | mCmAmAmCmGfAmCfUmCm |
| | | | | | | CmAmAmGpsmUpsmA |
| 136 | 267 | Sense | UGGAGUCGUUGGU | 136 | 581 | mUpsmGpsmGmAmGmUfCmG |
| | | | GAAGUUUU | | | fUfUfGmGmUmGmAmAmGm |
| | | | | | | UmUmUpsmU- |
| | | | | | | GalNAc4psGalNAc4psGalNAc4 |
| | 293 | Anti- | AAAACUUCACCAA | | 609 | mApsfApsmAmAmCfUmUmC |
| | | Sense | CGACUCCAUU | | | mAmCmCmAmAfCmGfAmCm |
| | | | | | | UmCmCmApsmUpsmU |
| 137 | 268 | Sense | GGUGAAGUUUUUC | 137 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 294 | Anti- | AGGAAUGAAAAAC | | 610 | vmApsfGpsmGmAmAfUmGmA |
| | | Sense | UUCACCAA | | | mAmAmAmAmCfUmUfCmAm |
| | | | | | | CmCpsmApsmA |
| 138 | 269 | Sense | GGUGAAGUUUUUC | 138 | 583 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 611 | mApsfGpsmGmAmAfUmGmA |
| | | Sense | UUCACCAA | | | mAmAmAmAmCfUmUfCmAm |
| | | | | | | CmCpsmApsmA |
| 139 | 270 | Sense | UUGGUGAAGUUUU | 139 | 584 | mUpsmUpsmGmGmUmGfAmA |
| | | | UCAUUCCU | | | fGfUfUmUmUmUmCmAmUm |
| | | | | | | UmCmCmU- |
| | | | | | | GalNAc4psGalNAc4psGalNAc4 |
| | 296 | Anti- | AGGAAUGAAAAAC | | 612 | vmApsfGpsmGmAmAfUmGmA |
| | | Sense | UUCACCAACG | | | mAmAmAmAmCfUmUfCmAm |
| | | | | | | CmCmAmApsmCpsmG |
| 140 | 271 | Sense | UUGGUGAAGUUUU | 140 | 585 | mUpsmUpsmGmGmUmGfAmA |
| | | | UCAUUCCU | | | fGfUfUmUmUmUmCmAmUm |
| | | | | | | UmCmCmU- |
| | | | | | | GalNAc4psGalNAc4psGalNAc4 |
| | 297 | Anti- | AGGAAUGAAAAAC | | 613 | mApsfGpsmGmAmAfUmGmA |
| | | Sense | UUCACCAACG | | | mAmAmAmAmCfUmUfCmAm |
| | | | | | | CmCmAmApsmCpsmG |
| 141 | 272 | Sense | UAUUGGUUCUGUG | 141 | 586 | mUpsmApsmUmUmGmGfUmU |
| | | | GGAUAUUA | | | fCfUfGmUmGmGmGmAmUm |
| | | | | | | AmUmUmA- |
| | | | | | | GalNAc4psGalNAc4psGalNAc4 |
| | 298 | Anti- | UAAUAUCCCACAG | | 614 | mUpsfApsmAmUmAfUmCmC |
| | | Sense | AACCAAUAUG | | | mCmAmCmAmGfAmAfCmCm |
| | | | | | | AmAmUmApsmUpsmG |
| 142 | 273 | Sense | UAUUGGUUCUGUG | 142 | 587 | mUpsmApsmUmUmGmGfUmU |
| | | | GGAUAUUA | | | fCfUfGmUmGmGmGmAmUm |
| | | | | | | AmUmUmA- |
| | | | | | | GalNAc4psGalNAc4psGalNAc4 |
| | 299 | Anti- | UAAUAUCCCACAG | | 615 | vmUpsfApsmAmUmAfUmCmC |
| | | Sense | AACCAAUAUG | | | mCmAmCmAmGfAmAfCmCm |
| | | | | | | AmAmUmApsmUpsmG |
| 143 | 274 | Sense | UAUUGGUUCUGUG | 143 | 588 | mUpsmApsmUmUmGmGfUmU |
| | | | GGAUAUUA | | | fCfUfGmUmGmGmGmAmUm |
| | | | | | | AmUmUmA- |
| | | | | | | GalNAc4psGalNAc4psGalNAc4 |
| | 300 | Anti- | AAAUAUCCCACAG | | 616 | vmApsfApsmAmUmAfUmCmC |
| | | Sense | AACCAAUAUG | | | mCmAmCmAmGfAmAfCmCm |
| | | | | | | AmAmUmApsmUpsmG |
| 144 | 275 | Sense | UUGGGAAAAACUG | 144 | 589 | mUpsmUpsmGmGmGmAfAmA |
| | | | GUAUCAAA | | | fAfAfCmUmGmGmUmAmUm |
| | | | | | | CmAmAmA- |
| | | | | | | GalNAc4psGalNAc4psGalNAc4 |
| | 301 | Anti- | UUUGAUACCAGUU | | 617 | mUpsfUpsmUmGmAfUmAmC |
| | | Sense | UUUCCCAAGG | | | mCmAmGmUmUfUmUfUmCm |
| | | | | | | CmCmAmApsmGpsmG |
| 145 | 276 | Sense | UUGGGAAAAACUG | 145 | 590 | mUpsmUpsmGmGmGmAfAmA |
| | | | GUAUCAAA | | | fAfAfCmUmGmGmUmAmUm |
| | | | | | | CmAmAmA- |
| | | | | | | GalNAc4psGalNAc4psGalNAc4 |
| | 302 | Anti- | UUUGAUACCAGUU | | 618 | vmUpsfUpsmUmGmAfUmAmC |
| | | Sense | UUUCCCAAGG | | | mCmAmGmUmUfUmUfUmCm |
| | | | | | | CmCmAmApsmGpsmG |
| 146 | 277 | Sense | UUGGGAAAAACUG | 146 | 591 | mUpsmUpsmGmGmGmAfAmA |
| | | | GUAUCAAA | | | fAfAfCmUmGmGmUmAmUm |
| | | | | | | CmAmAmA- |
| | | | | | | GalNAc4psGalNAc4psGalNAc4 |
| | 303 | Anti- | AUUGAUACCAGUU | | 619 | vmApsfUpsmUmGmAfUmAmC |
| | | Sense | UUUCCCAAGG | | | mCmAmGmUmUfUmUfUmCm |
| | | | | | | CmCmAmApsmGpsmG |
| 147 | 278 | Sense | ACCAAUAAGAAAA | 147 | 592 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 304 | Anti- | AAAAAUCAUUUUC | | 620 | mApsfApsmAmAmAfUmCmA |
| | | Sense | UUAUUGGUAA | | | mUmUmUmUmCfUmUfAmUm |
| | | | | | | UmGmGmUpsmApsmA |
| 148 | 279 | Sense | ACCAAUAAGAAAA | 148 | 593 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 305 | Anti- | AAAAAUCAUUUUC | | 621 | vmApsfApsmAmAmAfUmCmA |
| | | Sense | UUAUUGGUAA | | | mUmUmUmUmCfUmUfAmUm |
| | | | | | | UmGmGmUpsmApsmA |
| 149 | 280 | Sense | ACCAAUAAGAAAA | 149 | 594 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 306 | Anti- | UAAAAUCAUUUUC | | 622 | vmUpsfApsmAmAmAfUmCmA |
| | | Sense | UUAUUGGUAA | | | mUmUmUmUmCfUmUfAmUm |
| | | | | | | UmGmGmUpsmApsmA |
| 150 | 281 | Sense | UGGUAUCAAAACC | 150 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 307 | Anti- | AGACAUGAGGUUU | | 623 | mApsfGpsmAmCmAfUmGmA |
| | | Sense | UGAUACCAUU | | | mGmGmUmUmUfUmGfAmUm |
| | | | | | | AmCmCmApsmUpsmU |
| 151 | 282 | Sense | UGGUAUCAAAACC | 151 | 596 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 624 | vmApsfGpsmAmCmAfUmGmA |
| | | Sense | UGAUACCAGU | | | mGmGmUmUmUfUmGfAmUm |
| | | | | | | AmCmCmApsmGpsmU |
| 152 | 283 | Sense | GUAUCAAAACCUC | 152 | 597 | mGpsmUpsmAmUmCmAfAmA |
| | | | AUGUCUCU | | | fAfCfCmUmCmAmUmGmUmC |
| | | | | | | mUmCmU- |
| | | | | | | GalNAc4psGalNAc4psGalNAc4 |
| | 309 | Anti- | AGAGACAUGAGGU | | 625 | mApsfGpsmAmGmAfCmAmU |
| | | Sense | UUUGAUACUU | | | mGmAmGmGmUfUmUfUmGm |
| | | | | | | AmUmAmCpsmUpsmU |
| 153 | 284 | Sense | GUAUCAAAACCUC | 153 | 598 | mGpsmUpsmAmUmCmAfAmA |
| | | | AUGUCUCU | | | fAfCfCmUmCmAmUmGmUmC |
| | | | | | | mUmCmU- |
| | | | | | | GalNAc4psGalNAc4psGalNAc4 |
| | 310 | Anti- | AGAGACAUGAGGU | | 626 | mApsfGpsmAmGmAfCmAmU |
| | | Sense | UUUGAUACCA | | | mGmAmGmGmUfUmUfUmGm |
| | | | | | | AmUmAmCpsmCpsmA |
| 154 | 285 | Sense | GUAUCAAAACCUC | 154 | 599 | mGpsmUpsmAmUmCmAfAmA |
| | | | AUGUCUCU | | | fAfCfCmUmCmAmUmGmUmC |
| | | | | | | mUmCmU- |
| | | | | | | GalNAc4psGalNAc4psGalNAc4 |
| | 311 | Anti- | AGAGACAUGAGGU | | 627 | vmApsfGpsmAmGmAfCmAmU |
| | | Sense | UUUGAUACCA | | | mGmAmGmGmUfUmUfUmGm |
| | | | | | | AmUmAmCpsmCpsmA |
| 155 | 286 | Sense | GUAUCAAAACCUC | 155 | 600 | mGpsmUpsmAmUmCmAfAmA |
| | | | AUGUCUCU | | | fAfCfCmUmCmAmUmGmUmC |
| | | | | | | mUmCmU- |
| | | | | | | GalNAc4psGalNAc4psGalNAc4 |
| | 312 | Anti- | UGAGACAUGAGGU | | 628 | vmUpsfGpsmAmGmAfCmAmU |
| | | Sense | UUUGAUACCA | | | mGmAmGmGmUfUmUfUmGm |
| | | | | | | AmUmAmCpsmCpsmA |
| 156 | 287 | Sense | UGAAGUCGUAAGA | 156 | 601 | mUpsmGpsmAmAmGmUfCmG |
| | | | AGUCUGAU | | | fUfAfAmGmAmAmGmUmCm |
| | | | | | | UmGmApsmU- |
| | | | | | | GalNAc4psGalNAc4psGalNAc4 |
| | 313 | Anti- | AUCAGACUUCUUA | | 629 | mApsfUpsmCmAmGfAmCmU |
| | | Sense | CGACUUCAUU | | | mUmCmUmUmAfCmGfAmCm |
| | | | | | | UmUmCmApsmUpsmU |
| 157 | 262 | Sense | CUUGGAGUCGUUG | 157 | 576 | mCpsmUpsmUmGmGmAfGmU |
| | | | GUGAAGUU | | | fCfGfUmUmGmGmUmGmAm |
| | | | | | | AmGmUpsmU- |
| | | | | | | GalNAc4psGalNAc4psGalNAc4 |
| | 105 | Anti- | AACUUCACCAACG | | 450 | vmApsfApsmCmUmUfCmAmC |
| | | Sense | ACUCCAAGUU | | | mCmAmAmCmGfAmCfUmCm |
| | | | | | | CmAmAmGpsmUpsmU |
| 158 | 287 | Sense | UGAAGUCGUAAGA | 158 | 601 | mUpsmGpsmAmAmGmUfCmG |
| | | | AGUCUGAU | | | fUfAfAmGmAmAmGmUmCm |
| | | | | | | UmGmApsmU- |
| | | | | | | GalNAc4psGalNAc4psGalNAc4 |
| | 156 | Anti- | AUCAGACUUCUUA | | 501 | vmApsfUpsmCmAmGfAmCmU |
| | | Sense | CGACUUCAUU | | | mUmCmUmUmAfCmGfAmCm |
| | | | | | | UmUmCmApsmUpsmU |
| 159 | 314 | Sense | UUGGUGAAGUUUU | 159 | 602 | mUpsmUpsmGmGmUmGfAmA |
| | | | UCAUUCCU | | | fGfUfUmUmUmUmCmAmUm |
| | | | | | | UmCmCpsmU- |
| | | | | | | GalNAc4psGalNAc4psGalNAc4 |
| | 107 | Anti- | AGGAAUGAAAAAC | | 452 | vmApsfGpsmGmAmAfUmGmA |
| | | Sense | UUCACCAAUU | | | mAmAmAmAmCfUmUfCmAm |
| | | | | | | CmCmAmApsmUpsmU |
| 160 | 315 | Sense | ACCAAUAAGAAAA | 160 | 603 | mApsmCpsmCmAmAmUfAmA |
| | | | UGAUUUUU | | | fGfAfAmAmAmUmGmAmUm |
| | | | | | | UmUmUpsmU- |
| | | | | | | GalNAc4psGalNAc4psGalNAc4 |
| | 171 | Anti- | AAAAAUCAUUUUC | | 516 | vmApsfApsmAmAmAfUmCmA |
| | | Sense | UUAUUGGUUU | | | mUmUmUmUmCfUmUfAmUm |
| | | | | | | UmGmGmUpsmUpsmU |
| 161 | 281 | Sense | UGGUAUCAAAACC | 161 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 192 | Anti- | AGACAUGAGGUUU | | 537 | vmApsfGpsmAmCmAfUmGmA |
| | | Sense | UGAUACCAUU | | | mGmGmUmUmUfUmGfAmUm |
| | | | | | | AmCmCmApsmUpsmU |
| 162 | 286 | Sense | GUAUCAAAACCUC | 162 | 600 | mGpsmUpsmAmUmCmAfAmA |
| | | | AUGUCUCU | | | fAfCfCmUmCmAmUmGmUmC |
| | | | | | | mUmCmU- |
| | | | | | | GalNAc4psGalNAc4psGalNAc4 |
| | 193 | Anti- | AGAGACAUGAGGU | | 538 | vmApsfGpsmAmGmAfCmAmU |
| | | Sense | UUUGAUACCUU | | | mGmAmGmGmUfUmUfUmGm |
| | | | | | | AmUmAmCpsmUpsmU |
| 163 | 267 | Sense | UGGAGUCGUUGGU | 163 | 581 | mUpsmGpsmGmAmGmUfCmG |
| | | | GAAGUUUU | | | fUfUfGmGmUmGmAmAmGm |
| | | | | | | UmUmUpsmU- |
| | | | | | | GalNAc4psGalNAc4psGalNAc4 |
| | 106 | Anti- | AAAACUUCACCAA | | 451 | vmApsfApsmAmAmCfUmUmC |
| | | Sense | CGACUCCAUU | | | mAmCmCmAmAfCmGfAmCm |
| | | | | | | UmCmCmApsmUpsmU |
| 164 | 268 | Sense | GGUGAAGUUUUUC | 164 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 630 | vmApsfGpsmGmAmAfUunGm |
| | | Sense | UUCACCAA | | | AmAmAmAmAmCfUmUfCmA |
| | | | | | | mCmCpsmApsmA |
| 165 | 268 | Sense | GGUGAAGUUUUUC | 165 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 631 | vmApsfGpsmGmAmAfUmun34 |
| | | Sense | UUCACCAA | | | GmAmAmAmAmAmCfUmUfC |
| | | | | | | mAmCmCpsmApsmA |
| 166 | 268 | Sense | GGUGAAGUUUUUC | 166 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 632 | v- |
| | | Sense | UUCACCAA | | | munUpsfGpsmGmAmAfUmGm |
| | | | | | | AmAmAmAmAmCfUmUfCmA |
| | | | | | | mCmCpsmApsmA |
| 167 | 268 | Sense | GGUGAAGUUUUUC | 167 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 633 | 4hUpsfGpsmGmAmAfUmGmA |
| | | Sense | UUCACCAA | | | mAmAmAmAmCfUmUfCmAm |
| | | | | | | CmCpsmApsmA |
| 168 | 268 | Sense | GGUGAAGUUUUUC | 168 | 582 | mGpsmGpsmUmGfAmAfGfUf |
| | | | AUUCCU | | | UmUmUmUmCmAmUmUmCm |
| | | | | | | CmU- |
| | | | | | | GalNAc4psGalNAc4psGalNAc4 |
| | 295 | Anti- | AGGAAUGAAAAAC | | 634 | c2o- |
| | | Sense | UUCACCAA | | | 4hUpsfGpsmGmAmAfUmGmA |
| | | | | | | mAmAmAmAmCfUmUfCmAm |
| | | | | | | CmCpsmApsmA |
| 169 | 71 | Sense | ACCAAUAAGAAAA | 169 | 592 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 306 | Anti- | UAAAAUCAUUUUC | | 635 | v- |
| | | Sense | UUAUUGGUAA | | | munUpsfApsmAmAmAfUmCm |
| | | | | | | AmUmUmUmUmCfUmUfAmU |
| | | | | | | mUmGmGmUpsmApsmA |
| 170 | 71 | Sense | ACCAAUAAGAAAA | 170 | 592 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 306 | Anti- | UAAAAUCAUUUUC | | 636 | 4hUpsfApsmAmAmAfUmCmA |
| | | Sense | UUAUUGGUAA | | | mUmUmUmUmCfUmUfAmUm |
| | | | | | | UmGmGmUpsmApsmA |
| 171 | 71 | Sense | ACCAAUAAGAAAA | 171 | 592 | mApsmCpsmCmAmAmUfAmA |
| | | | UGAUUUUU | | | fGfAfAmAmAmUmGmAmUm |
| | | | | | | UmUmUmU- |
| | | | | | | GalNAc4psGalNAc4psGalNAc4 |
| | 306 | Anti- | UAAAAUCAUUUUC | | 637 | c2o- |
| | | Sense | UUAUUGGUAA | | | 4hUpsfApsmAmAmAfUmCmA |
| | | | | | | mUmUmUmUmCfUmUfAmUm |
| | | | | | | UmGmGmUpsmApsmA |
| 172 | 71 | Sense | ACCAAUAAGAAAA | 172 | 638 | mApsmCpsmCmAmAmUfAmA |
| | | | UGAUUUUU | | | fGfAfAmAmAmUmGmAmUm |
| | | | | | | UmUmUpsmU- |
| | | | | | | GalNAc4psGalNAc4psGalNAc4 |
| | 306 | Anti- | UAAAAUCAUUUUC | | 621 | vmApsfApsmAmAmAfUmCmA |
| | | Sense | UUAUUGGUAA | | | mUmUmUmUmCfUmUfAmUm |
| | | | | | | UmGmGmUpsmApsmA |
| 173 | 92 | Sense | UGGUAUCAAAACC | 173 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 639 | vmApsfGpsmAmCmAfUunGm |
| | | Sense | UGAUACCAGU | | | AmGmGmUmUmUfUmGfAmU |
| | | | | | | mAmCmCmApsmGpsmU |
| 174 | 92 | Sense | UGGUAUCAAAACC | 174 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 640 | vmApsfGpsmAmCmAfUmun34 |
| | | Sense | UGAUACCAGU | | | GmAmGmGmUmUmUfUmGfA |
| | | | | | | mUmAmCmCmApsmGpsmU |
| 175 | 92 | Sense | UGGUAUCAAAACC | 175 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 641 | vmApsfGpsmAmCmA3ohUmG |
| | | Sense | UGAUACCAGU | | | mAmGmGmUmUmUfUmGfAm |
| | | | | | | UmAmCmCmApsmGpsmU |
| 176 | 92 | Sense | UGGUAUCAAAACC | 176 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 642 | vmApsfGpsmAmCmAfU3ohGm |
| | | Sense | UGAUACCAGU | | | AmGmGmUmUmUfUmGfAmU |
| | | | | | | mAmCmCmApsmGpsmU |
| 177 | 92 | Sense | UGGUAUCAAAACC | 177 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 643 | vmApsfGpsmAmCmA3mUmG |
| | | Sense | UGAUACCAGU | | | mAmGmGmUmUmUfUmGfAm |
| | | | | | | UmAmCmCmApsmGpsmU |
| 178 | 92 | Sense | UGGUAUCAAAACC | 178 | 595 | mUpsmGpsmGmUmAmUfCmA |
| | | | UCAUGUCU | | | fAfAfAmCmCmUmCmAmUm |
| | | | | | | GmUmCmU- |
| | | | | | | GalNAc4psGalNAc4psGalNAc4 |
| | 308 | Anti- | AGACAUGAGGUUU | | 644 | vmApsfGpsmAmCmAfU3mGm |
| | | Sense | UGAUACCAGU | | | AmGmGmUmUmUfUmGfAmU |
| | | | | | | mAmCmCmApsmGpsmU |

TABLE 13

| SEQ ID | | |
|---|---|---|
| NO: | Description | Sequence |
| 261 | 17β-HSD | ATGAACATCATCCTAGAAATCCTTCTGCTTCTGATCACCATCAT |
| | type 13 | CTACTCCTACTTGGAGTCGTTGGTGAAGTTTTTCATTCCTCAGA |
| | coding | GGAGAAAATCTGTGGCTGGGGAGATTGTTCTCATTACTGGAGC |
| | sequence | TGGGCATGGAATAGGCAGGCAGACTACTTATGAATTTGCAAA |
| | (Genbank | ACGACAGAGCATATTGGTTCTGTGGGATATTAATAAGCGCGGT |
| | Accession | GTGGAGGAAACTGCAGCTGAGTGCCGAAAACTAGGCGTCACT |
| | No. NM | GCGCATGCGTATGTGGTAGACTGCAGCAACAGAGAAGAGATC |
| | 178135.5) | TATCGCTCTCTAAATCAGGTGAAGAAAGAAGTGGGTGATGTAA |
| | (nucleotides | CAATCGTGGTGAATAATGCTGGGACAGTATATCCAGCCGATCT |
| | 42 to 944) | TCTCAGCACCAAGGATGAAGAGATTACCAAGACATTTGAGGTC |
| | | AACATCCTAGGACATTTTTGGATCACAAAAGCACTTCTTCCAT |
| | | CGATGATGGAGAGAAATCATGGCCACATCGTCACAGTGGCTTC |
| | | AGTGTGCGGCCACGAAGGGATTCCTTACCTCATCCCATATTGT |
| | | TCCAGCAAATTTGCCGCTGTTGGCTTTCACAGAGGTCTGACAT |
| | | CAGAACTTCAGGCCTTGGGAAAAACTGGTATCAAAACCTCATG |
| | | TCTCTGCCCAGTTTTTGTGAATACTGGGTTCACCAAAAATCCA |
| | | AGCACAAGATTATGGCCTGTATTGGAGACAGATGAAGTCGTA |
| | | AGAAGTCTGATAGATGGAATACTTACCAATAAGAAAATGATTT |
| | | TTGTTCCATCGTATATCAATATCTTTCTGAGACTACAGAAGTTT |
| | | CTTCCTGAACGCGCCTCAGCGATTTTAAATCGTATGCAGAATA |
| | | TTCAATTTGAAGCAGTGGTTGGCCACAAAATCAAAATGAAATG |
| | | A |

### Exemplary Embodiments

Exemplary embodiments are provided below:

Embodiment 1. A short interfering nucleic acid (siNA) molecule comprising: (a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and (b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence: (i) is 15 to 30 nucleotides in length; and (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide.

Embodiment 2. In any preceding embodiment, the first nucleotide sequence may comprise a nucleotide sequence of any one of SEQ ID NOs: 1-100, 201-230, 262-287, 314, or 315.

Embodiment 3. In any preceding embodiment, the second nucleotide sequence may comprise a nucleotide sequence of any one of SEQ ID NOs: 101-200, or 231-260, 288-313.

Embodiment 4. In any preceding embodiment, the target gene may be hydroxysteroid 17-beta dehydrogenase 13 (HSD17B13). In any embodiment, the siNA may reduce or inhibit the production of a hydroxysteroid dehydrogenase. In any embodiment, the siNA molecule decreases expression or activity of HSD17B13.

Embodiment 5. In any preceding embodiment, the first nucleotide sequence comprises 16, 17, 18, 19, 20, 21, 22, 23, or more modified nucleotides independently selected from a 2′-O-methyl nucleotide and a 2′-fluoro nucleotide.

Embodiment 6. In any preceding embodiment, the 70%, 75%, 80%, 85%, 90%, 95% or 100% of the nucleotides in the first nucleotide sequence are modified nucleotides independently selected from a 2′-O-methyl nucleotide and a 2′-fluoro nucleotide.

Embodiment 7. In any preceding embodiment, at least 2, 3, 4, 5, or 6 modified nucleotides of the first nucleotide sequence are 2′-fluoro nucleotides.

Embodiment 8. In any preceding embodiment, no more than 10, 9, 8, 7, 6, 5, 4, 3, or 2 modified nucleotides of the first nucleotide sequence are 2′-fluoro nucleotides.

Embodiment 9. In any preceding embodiment, at least 10, 11, 12, 13, 14, 15, 16, 17, 18, 19, 20, 21, or 22 modified nucleotides of the first nucleotide sequence are 2′-O-methyl nucleotides.

Embodiment 10. In any preceding embodiment, no more than 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides.

Embodiment 11. In any preceding embodiment, the second nucleotide sequence comprises 16, 17, 18, 19, 20, 21, 22, 23, or more modified nucleotides independently selected from a 2′-O-methyl nucleotide and a 2′-fluoro nucleotide.

Embodiment 12. In any preceding embodiment, 70%, 75%, 80%, 85%, 90%, 95% or 100% of the nucleotides in the second nucleotide sequence are modified nucleotides independently selected from a 2′-O-methyl nucleotide and a 2′-fluoro nucleotide.

Embodiment 13. In any preceding embodiment, at least 2, 3, 4, 5, or 6 modified nucleotides of the second nucleotide sequence are 2′-fluoro nucleotides.

Embodiment 14. In any preceding embodiment, less than or equal to 10, 9, 8, 7, 6, 5, 4, 3, or 2 modified nucleotides of the second nucleotide sequence are 2′-fluoro nucleotides.

Embodiment 15. In any preceding embodiment, at least 10, 11, 12, 13, 14, 15, 16, 17, 18, 19, 20, 21, or 22 modified nucleotides of the second nucleotide sequence are 2′-O-methyl nucleotides.

Embodiment 16. In any preceding embodiment, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides.

Embodiment 17. In any preceding embodiment, at least 1, 2, 3, 4, 5, 6, or 7 nucleotides at position 3, 5, 7, 8, 9, 10, 11, 12, and/or 17 from the 5′ end of the first nucleotide sequence is a 2′-fluoro nucleotide.

Embodiment 18. In any preceding embodiment, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide.

Embodiment 19. In any preceding embodiment, the nucleotides in the second nucleotide sequence are arranged in an alternating 1:3 modification pattern, and wherein 1 nucleotide is a 2′-fluoro nucleotide and 3 nucleotides are 2′-O-methyl nucleotides. In any embodiment, the alternating 1:3 modification pattern occurs 2-5 times. In any embodiment, at least two of the alternating 1:3 modification pattern occur consecutively. In any embodiment, at least two of the alternating 1:3 modification pattern occurs nonconsecutively. In any embodiment, at least 1, 2, 3, 4, or 5 alternating 1:3 modification pattern begins at nucleotide position 2, 6, 10, 14, and/or 18 from the 5′ end of the antisense strand.

Embodiment 20. In any preceding embodiment, the nucleotides in the second nucleotide sequence are arranged in an alternating 1:2 modification pattern. In any embodiment, 1 nucleotide is a 2′-fluoro nucleotide and 2 nucleotides are 2′-O-methyl nucleotides. In any embodiment, the alternating 1:2 modification pattern occurs 2-5 times. In any embodiment, at least two of the alternating 1:2 modification pattern occurs consecutively. In any embodiment, at least two of the alternating 1:2 modification pattern occurs nonconsecutively. In any embodiment, at least 1, 2, 3, 4, or 5 alternating 1:2 modification pattern begins at nucleotide position 2, 5, 8, 14, and/or 17 from the 5′ end of the antisense strand.

Embodiment 21. A short interfering nucleic acid (siNA) molecule represented by Formula (VIII):

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O-methyl nucleotide or a nucleotide comprising a 5′-stabilized end cap or a phosphorylation blocker; B is a 2′-fluoro nucleotide; C represents overhanging nucleotides and is a 2′-O-methyl nucleotide, deoxy nucleotide, or uracil; n¹=1-6 nucleotides in length; each n², n⁶, n⁸, q³, q⁵, q⁷, q⁹, q¹¹, and q¹² is independently 0-1 nucleotides in length; each n³ and n⁴ is independently 1-3 nucleotides in length; n⁵ is 1-10 nucleotides in length; n⁷ is 0-4 nucleotides in length; each n⁹, q¹, and q² is independently 0-2 nucleotides in length; q⁴ is 0-3 nucleotides in length; q⁶ is 0-5 nucleotides in length; q⁸ is 2-7 nucleotides in length; and q¹⁰ is 2-11 nucleotides in length.

Embodiment 22. In embodiment 21, the first nucleotide sequence may comprise a nucleotide sequence of any one of SEQ ID NOs: 1-100, 201-230, 262-287, 314, or 315.

Embodiment 23. In embodiment 21 or 22, the second nucleotide sequence may comprise a nucleotide sequence of any one of SEQ ID NOs: 101-200, or 231-260, 288-313.

Embodiment 24. In any one of embodiments 21-23, the siNA may reduce or inhibit the production of a hydroxysteroid dehydrogenase.

Embodiment 25. In any one of embodiments 21-24, the siNA may be represented by Formula (IX):

5′-A₂₋₆B₁A₁₋₃B₂₋₃A₂₋₁₀B₀₋₁A₀₋₄B₀₋₁A₀₋₂-3′
3′-C₂A₀₋₂B₀₋₁A₀₋₃B₀₋₁A₀₋₅B₀₋₁A₂₋₇B₁A₂₋₁₁B₁A₁-5′

wherein: the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides; the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides; each A is independently a 2′-O-methyl nucleotide or a nucleotide comprising a 5′-stabilized end cap or a phosphorylation blocker; B is a 2′-fluoro nucleotide; C represents overhanging nucleotides and is a 2′-O methyl nucleotide, deoxy nucleotide, or uracil.

Embodiment 26. In any one of embodiments 21-25, the (a) a sense strand comprising a first nucleotide sequence consisting of 17 to 23 nucleotides, wherein 2′-fluoro nucleotides are at positions 3, 7-9, 12, and 17 from the 5′ end of the first nucleotide sequence, and wherein 2′-O methyl nucleotides are at positions 1, 2, 4-6, 10, 11, and 13-16 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence.

Embodiment 27. In any one of embodiments 21-25, the (a) a sense strand comprising a first nucleotide sequence consisting of 17 to 23 nucleotides, wherein 2′-fluoro nucleotides are at positions 3, 7, 8, and 17 from the 5′ end of the first nucleotide sequence, and wherein 2′-O methyl nucleotides are at positions 1, 2, 4-6, and 9-16 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence.

Embodiment 28. In any one of embodiments 21-25, the (a) a sense strand comprising a first nucleotide sequence consisting of 17 to 23 nucleotides, wherein 2′-fluoro nucleotides are at positions 3, 7-9, and 17 from the 5′ end of the first nucleotide sequence, and wherein 2′-O methyl nucleotides are at positions 1, 2, 4-6, and 10-16 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 10, and 14 from the 5′ end of the second nucleotide sequence, and wherein 2′-O methyl nucleotides are at positions 1, 3-5, 7-9, 11-13, and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 19-21 from the 5′ end of the second nucleotide sequence.

Embodiment 29. In any one of embodiments 21-25, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 10, and 14 from the 5′ end of the second nucleotide sequence, and wherein 2′-O methyl nucleotides are at positions 1, 3-5, 7-9, 11-13 and 15-17 from the 5′ end of the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-fluoro nucleotide is at position 18 from the 5′ end of the second nucleotide sequence. In any embodiment, 2′-O methyl nucleotides are at positions 19-21 from the 5′ end of the second nucleotide sequence.

Embodiment 30. In any one of embodiments 21-25, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 5, 8, 14, and 17 from the 5′ end of the first nucleotide sequence, and wherein 2′-O methyl nucleotides are at positions 1, 3, 4, 6, 7, 9-13, 15, and 16 from the 5′ end of the first nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence.

Embodiment 31. In any one of embodiments 21-25, the (a) a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O methyl nucleotides are at positions 1-4, 6, and 10-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 14, and 16 from the 5′ end of the second nucleotide sequence, and wherein 2′-O methyl nucleotides are at positions 1, 3-5, 7-13, 15, and 17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, first nucleotide sequence consists of 19 nucleotides. In any embodiment, 2′-O methyl nucleotides are at positions 18 and 19 from the 5′ end of the first nucleotide sequence. In any embodiment, the second nucleotide sequence consists of 21 nucleotides. In any embodiment, 2′-O methyl nucleotides are at positions 18-21 or 19-21 from the 5′ end of the second nucleotide sequence.

Embodiment 32. In any one of embodiments 21-25, the (a) a sense strand comprising a first nucleotide sequence consisting of 17 to 23 nucleotides, wherein 2′-fluoro nucleotides are at positions 5, 9-11, and 14 from the 5′ end of the first nucleotide sequence, and wherein 2′-O-methyl nucleotides are at positions 1-4, 6-8, 12, 13, and 15-17 from the 5′ end of the first nucleotide sequence; and (b) an antisense strand comprising a second nucleotide sequence consisting of 17 to 23 nucleotides. In some embodiments, 2′-fluoro nucleotides are at positions 2 and 14 from the 5′ end of the second nucleotide sequence, and wherein 2′-O methyl nucleotides are at positions 1, 3-13, and 15-17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 21 nucleotides. In any embodiment, 2′-O methyl nucleotides are at positions 18, 20, and 21 from the 5′ end of the first nucleotide sequence. In any embodiment, 2′-fluoro nucleotide is at position 19 from the 5′ end of the first nucleotide sequence. In any embodiment, the second nucleotide sequence consists of 23 nucleotides. In any embodiment, 2′-O methyl nucleotides are at positions 18-23 from the 5′ end of the second nucleotide sequence.

Embodiment 33. In any one of embodiments 21-25, the (a) a sense strand comprising a first nucleotide sequence consisting of 17 to 23 nucleotides, wherein 2′-fluoro nucleotides are at positions 7 and 9-11 from the 5′ end of the first nucleotide sequence, and wherein 2′-O methyl nucleotides are at positions 1-6, 8, and 12-17 from the 5′ end of the first nucleotide sequence; and (b) an antisense strand comprising a second nucleotide sequence consisting of 17 to 23 nucleotides. In some embodiments, 2′-fluoro nucleotides are at positions 2, 6, 14, and 16 from the 5′ end of the second nucleotide sequence, and wherein 2′-O methyl nucleotides are at positions 1, 3-5, 7-13, 15, and 17 from the 5′ end the second nucleotide sequence. In some embodiments, the nucleotides in the second nucleotide sequence are arranged in an alternating 1:3 modification pattern, and wherein 1 nucleotide is a 2′-fluoro nucleotide and 3 nucleotides are 2′-O methyl nucleotides. In some embodiments, the nucleotides in the second nucleotide sequence are arranged in an alternating 1:2 modification pattern, and wherein 1 nucleotide is a 2′-fluoro nucleotide and 2 nucleotides are 2′-O methyl nucleotides. In any embodiment, the first nucleotide sequence consists of 21 nucleotides. In any embodiment, 2′-O methyl nucleotides are at positions 18-21 from the 5′ end of the first nucleotide sequence. In any embodiment, the second nucleotide sequence consists of 23 nucleotides. In any embodiment, 2′-O methyl nucleotides are at positions 18-21 from the 5′ end of the second nucleotide sequence.

Embodiment 34. In any proceeding embodiment, the sense strand may further comprises TT sequence adjacent to the first nucleotide sequence.

Embodiment 35. In any proceeding embodiment, the antisense strand may further comprises TT sequence adjacent to the first nucleotide sequence.

Embodiment 36. In any proceeding embodiment, the sense strand may further comprises at least 1, 2, 3, 4, 5, 6, 7, 8, 9, 10, 11, 12, 13, 14, 15 or more phosphorothioate internucleoside linkages.

Embodiment 37. In any proceeding embodiment, the antisense strand may further comprises at least 1, 2, 3, 4, 5, 6, 7, 8, 9, 10, 11, 12, 13, 14, 15 or more phosphorothioate internucleoside linkages.

Embodiment 38. In any proceeding embodiment, the first nucleotide sequence further comprises 1 or more phosphorothioate internucleoside linkages.

Embodiment 39. In any proceeding embodiment, the second nucleotide sequence further comprises 1 or more phosphorothioate internucleoside linkages.

Embodiment 40. In any proceeding embodiment, at least one phosphorothioate internucleoside linkage is between the nucleotides at positions 1 and 2 from the 5′ end of the first nucleotide sequence.

Embodiment 41. In any proceeding embodiment, at least one phosphorothioate internucleoside linkage is between the nucleotides at positions 2 and 3 from the 5′ end of the first nucleotide sequence.

Embodiment 42. In any proceeding embodiment, at least one phosphorothioate internucleoside linkage is between the nucleotides at positions 1 and 2 from the 5′ end of the second nucleotide sequence.

Embodiment 43. In any proceeding embodiment, at least one phosphorothioate internucleoside linkage is between the nucleotides at positions 2 and 3 from the 5′ end of the second nucleotide sequence.

Embodiment 44. In any proceeding embodiment, the first nucleotide sequence may further comprises 1 or more mesyl phosphoroamidate internucleoside linkages, the second nucleotide sequence further comprises 1 or more mesyl phosphoroamidate internucleoside linkages, or a combination thereof. In any embodiment, the sense strand comprises at least 1, 2, 3, 4, 5, 6, 7, 8, 9, 10, 11, 12, 13, 14, 15 or more mesyl phosphoroamidate internucleoside linkages. In any embodiment, the antisense strand further comprises at least 1, 2, 3, 4, 5, 6, 7, 8, 9, 10, 11, 12, 13, 14, 15 or more mesyl phosphoroamidate internucleoside linkages.

Embodiment 45. In embodiment 44, at least one mesyl phosphoroamidate internucleoside linkage is between the nucleotides at positions 1 and 2 from the 5′ end of the first nucleotide sequence and/or second nucleotide sequence.

Embodiment 46. In embodiment 44 or 45, at least one mesyl phosphoroamidate internucleoside linkage is between the nucleotides at positions 2 and 3 from the 5′ end of the first nucleotide sequence and/or second nucleotide sequence.

Embodiment 47. In any one of embodiments 44-46, at least one mesyl phosphoroamidate internucleoside linkage is between the nucleotides at positions 1 and 2 from the 3′ end of the first nucleotide sequence and/or second nucleotide sequence.

Embodiment 48. In any one of embodiments 44-47, at least one mesyl phosphoroamidate internucleoside linkage is between the nucleotides at positions 2 and 3 from the 3′ end of the first nucleotide sequence and/or second nucleotide sequence.

Embodiment 49. In any proceeding embodiment, the first nucleotide from the 5′ end of the first nucleotide sequence comprises a 5′ stabilizing end cap.

Embodiment 50. In any proceeding embodiment, the first nucleotide from the 5′ end of the second nucleotide sequence comprises a 5′ stabilizing end cap.

Embodiment 51. In embodiment 49 or 50, the 5′-stabilized end cap may be Formula (I′):

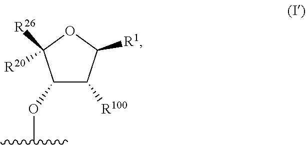

wherein: R¹ is a nucleobase, aryl, heteroaryl, or H, R²⁶ is

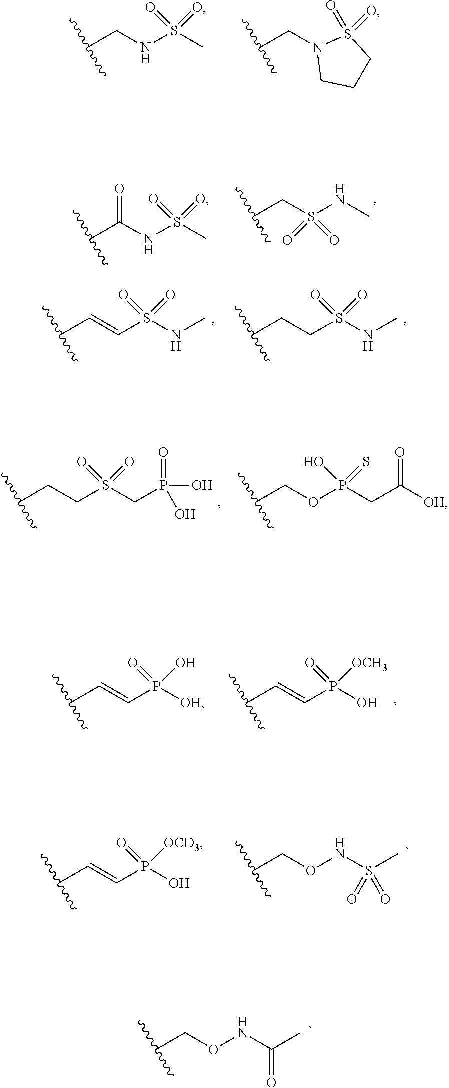

—CH═CD-Z, —CD═CH—Z, —CD═CD-Z, —(CR²¹R²²)ₙ—Z, or —(C₂-C₆ alkenylene)-Z and R²⁰ is hydrogen; or R² and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂ (CH₂)ₘP(O)(OH)₂, —SO₂NR²³R²⁵, —NR²³R²⁴, R²¹ and R²² are independently hydrogen or C₁-C₆ alkyl; R²¹ and R²² together form an oxo group; R²³ is hydrogen or C₁-C₆ alkyl; R²⁴ is-SO₂R²⁵ or —C(O)R²⁵; or R²³ and R²⁴ together with the nitrogen to which they are attached form a substituted or unsubstituted heterocyclic ring; R²⁵ is C₁-C₆ alkyl; R¹⁰⁰ is —OCH₃, —OCD₃, or —F; and m is 1, 2, 3, or 4. In some embodiments, the 5′-stabilized end cap may be selected from the group consisting of Formula (1) to Formula (15), Formula (9X) to Formula (12X), and Formula (9Y) to Formula (12Y):

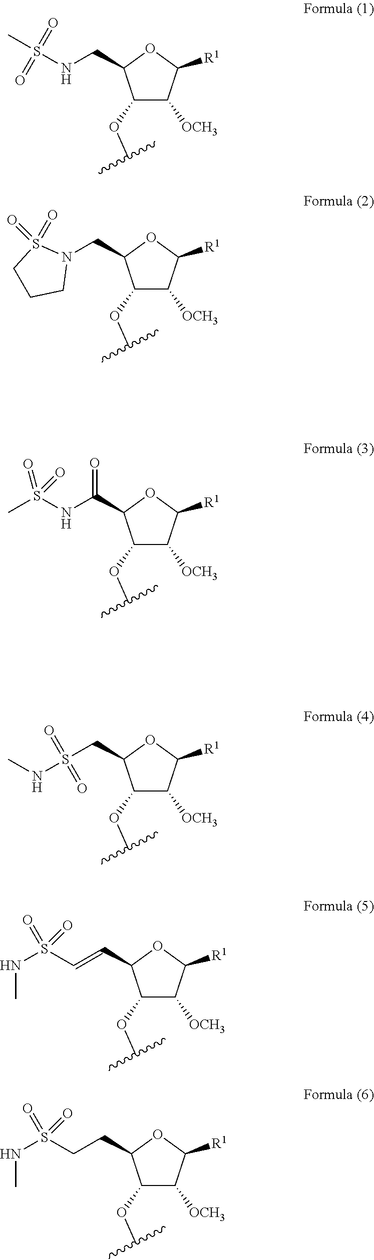

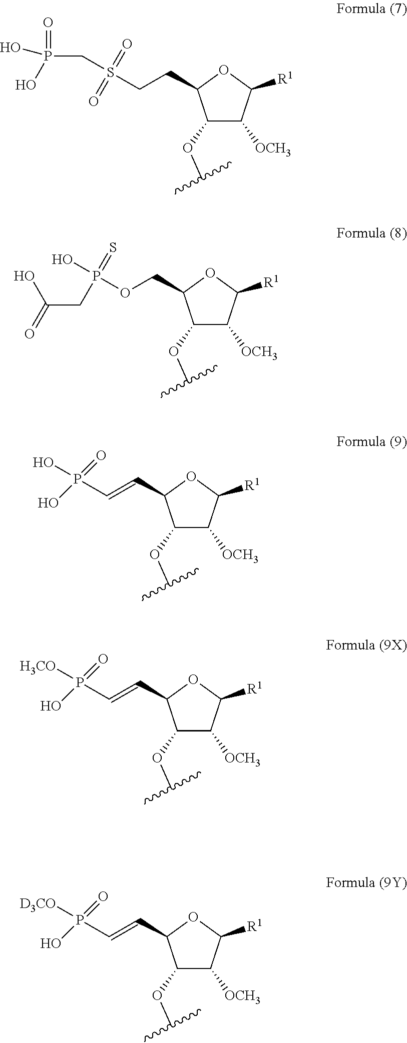

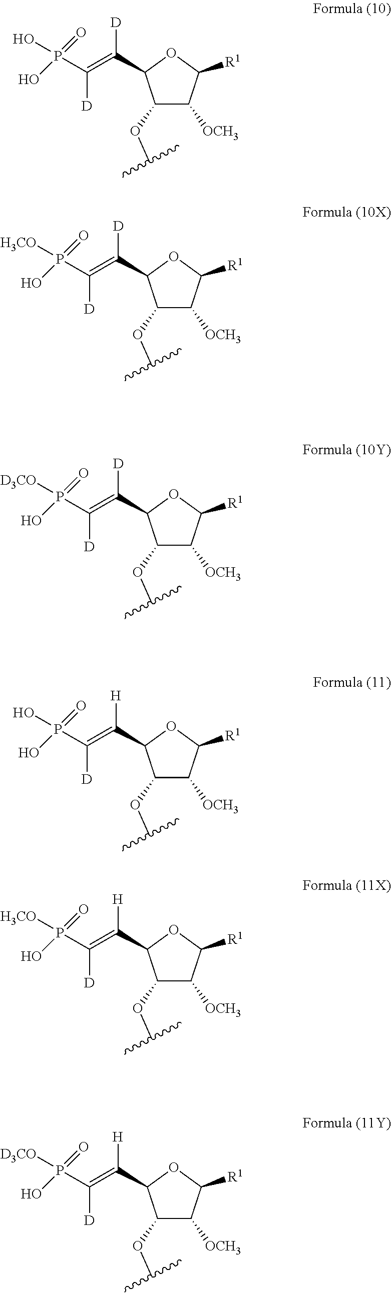

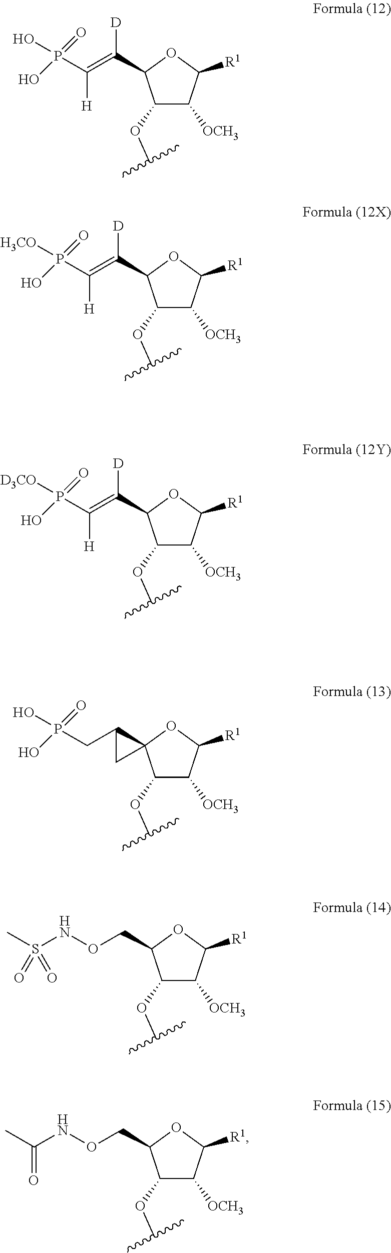

wherein R¹ is a nucleobase, aryl, heteroaryl, or H. In some embodiments, The 5′-stabilized end cap may be selected from the group consisting of Formulas (1A)-(15A), Formulas (9B)-(12B), Formulas (9AX)-(12AX), Formulas (9AY)-(12AY), Formulas (9BX)-(12BX), and Formulas (9BY)-(12BY):

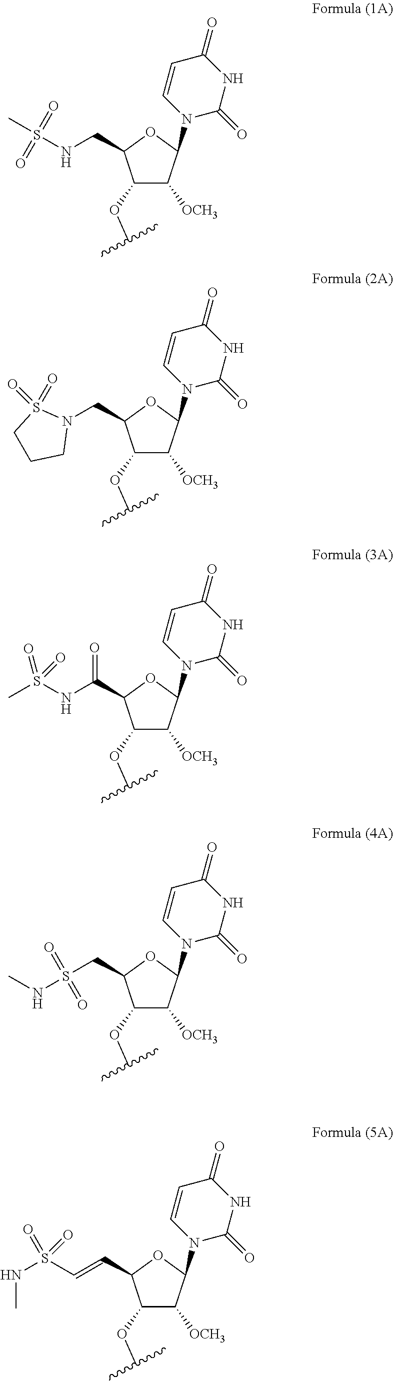 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC ; *O[C@H]1[C@@H](OC)[C@H](n2ccc(=O)[nH]c2=O)O[C@@H]1/C=C/S(=O)(=O)NC]

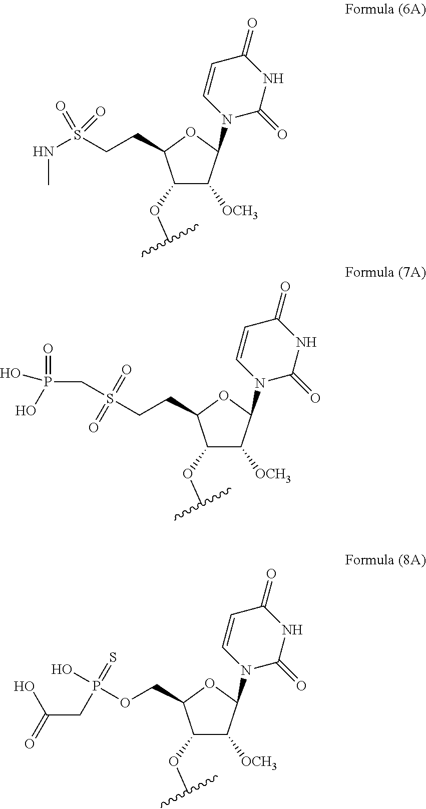 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2ccc(=O)[nH]c2=O)O[C@@H]1COP(O)(=S)CC(=O)O]

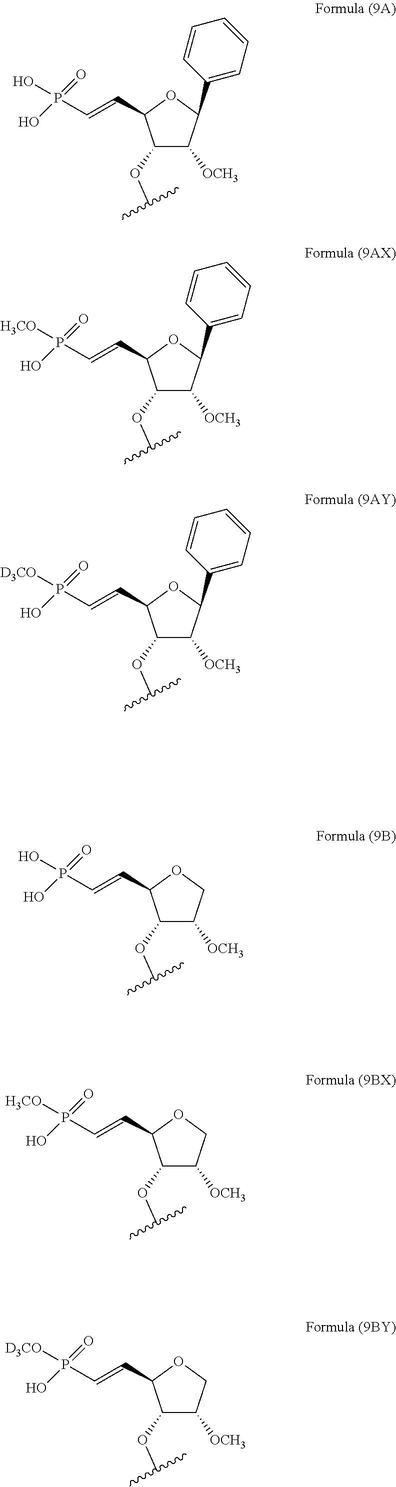

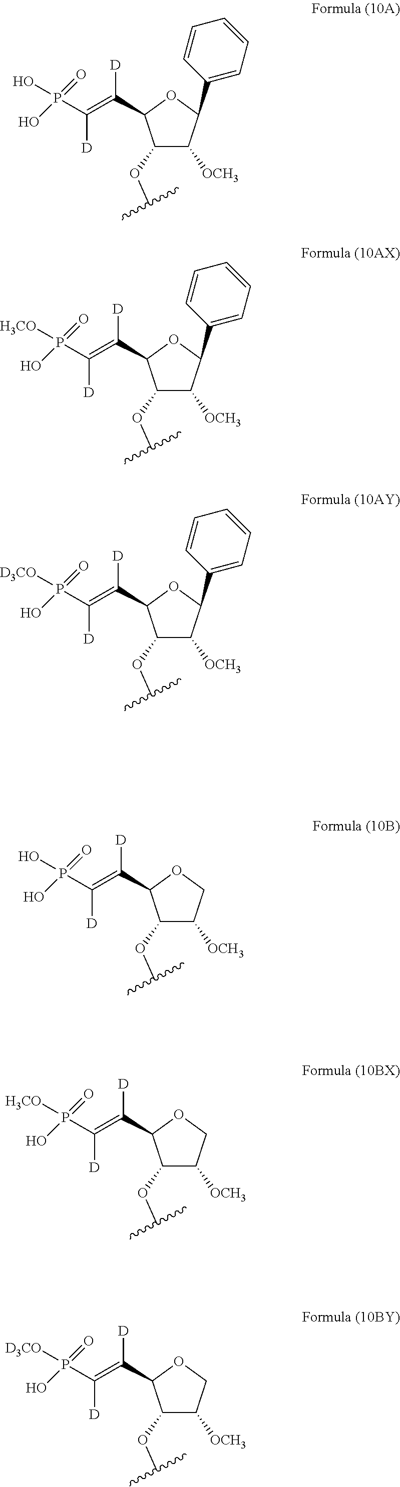

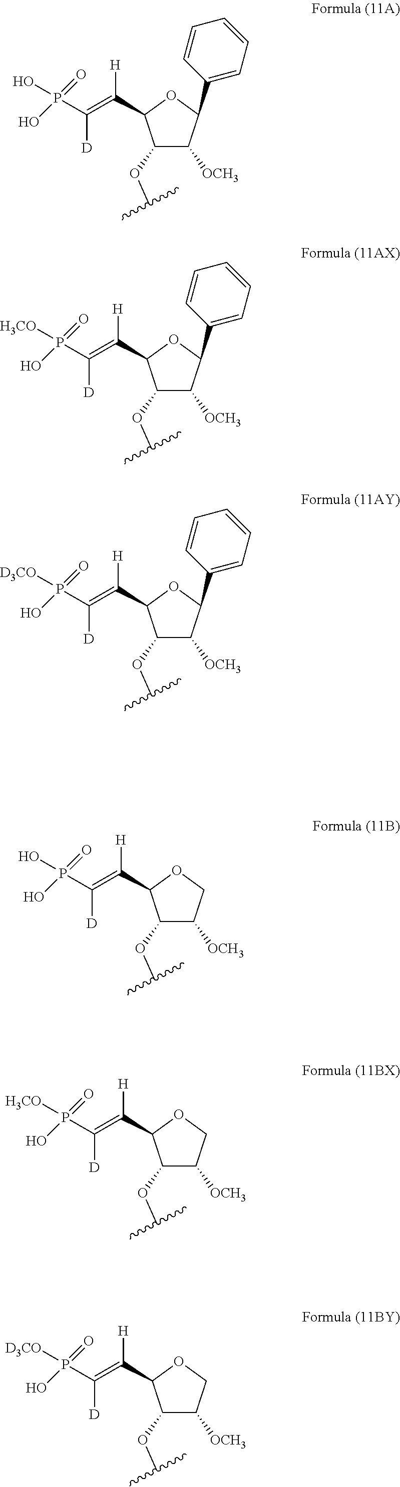

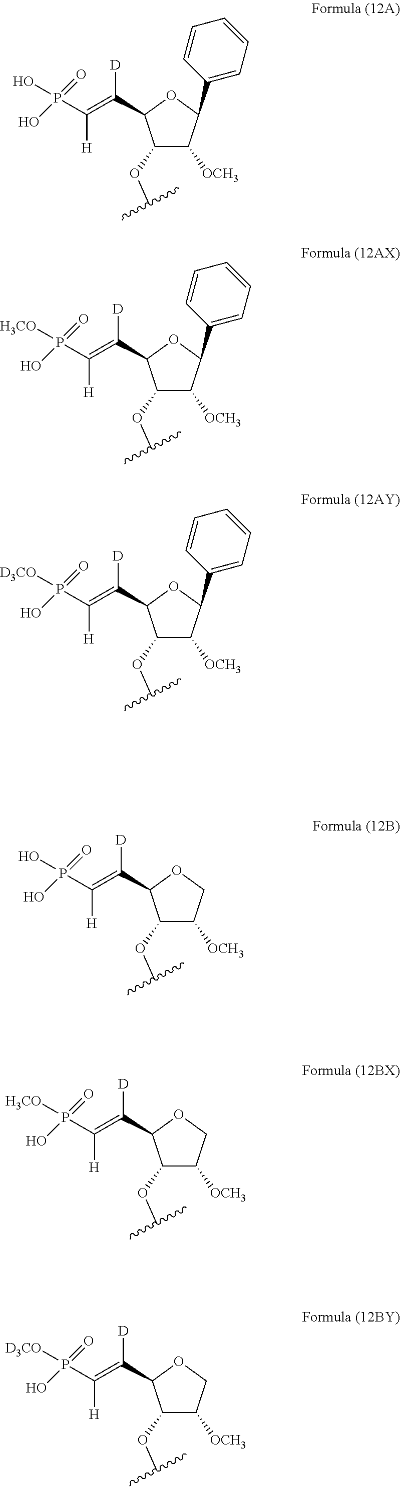

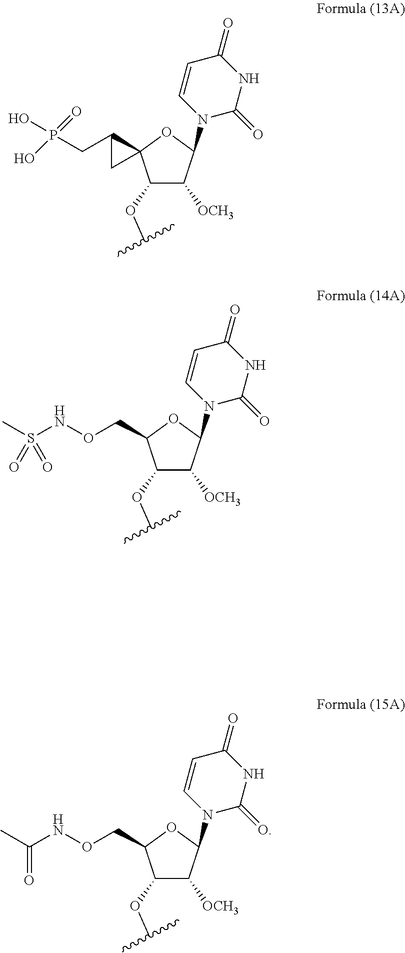

In some embodiments, The 5′-stabilized end cap may be selected from the group consisting of Formula (21) to Formula (35):

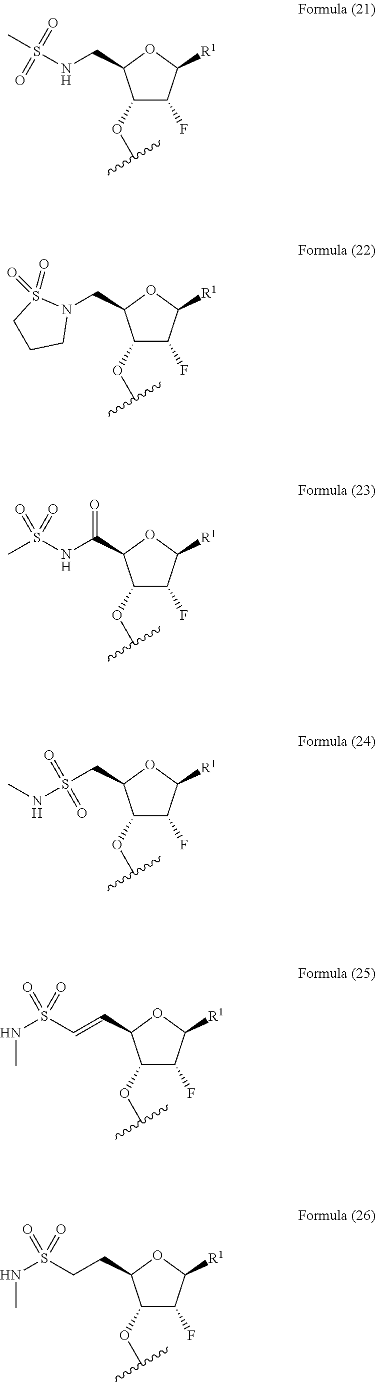

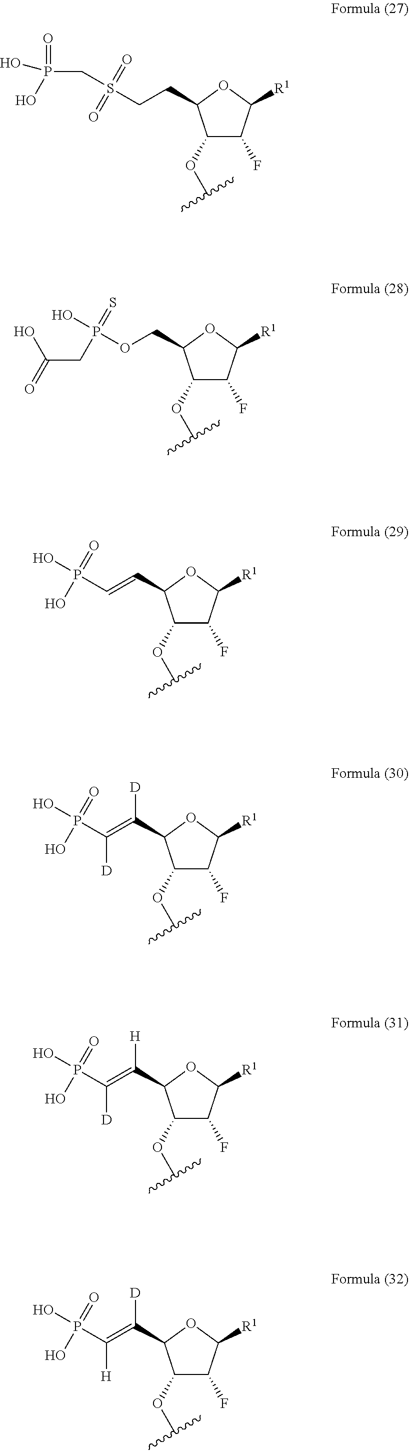

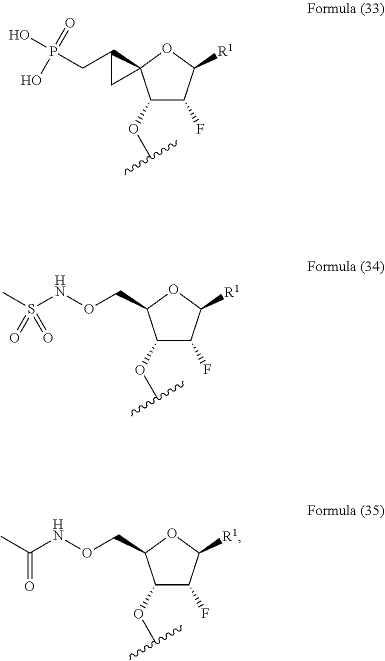

wherein R¹ is a nucleobase, aryl, heteroaryl, or H. In some embodiments, The 5′-stabilized end cap may be selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

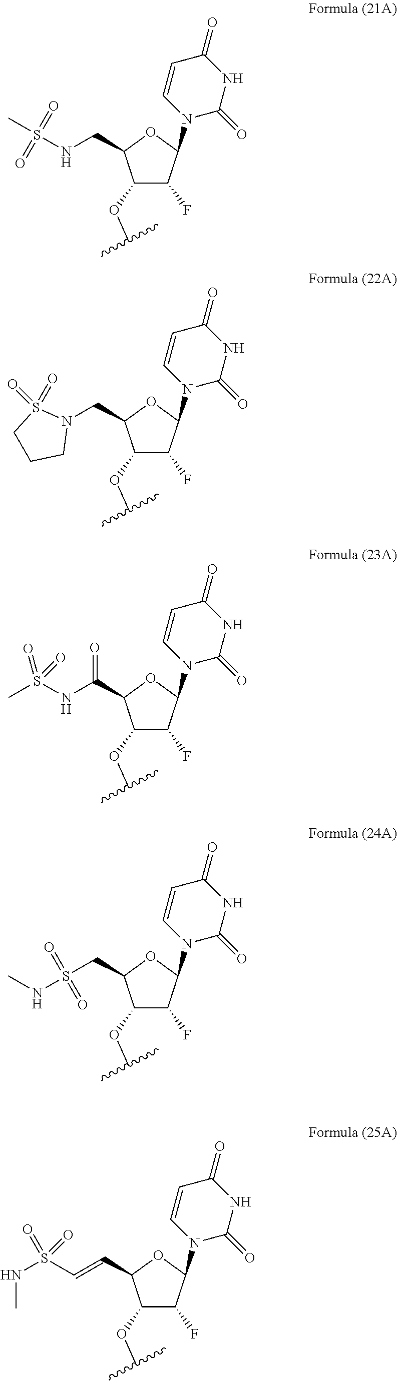

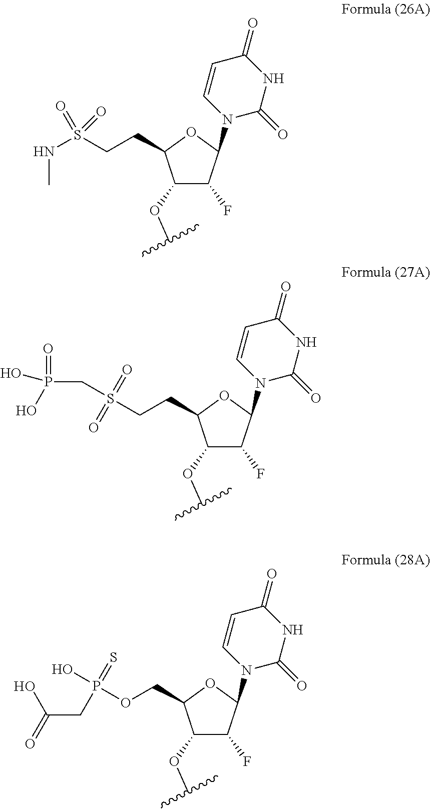

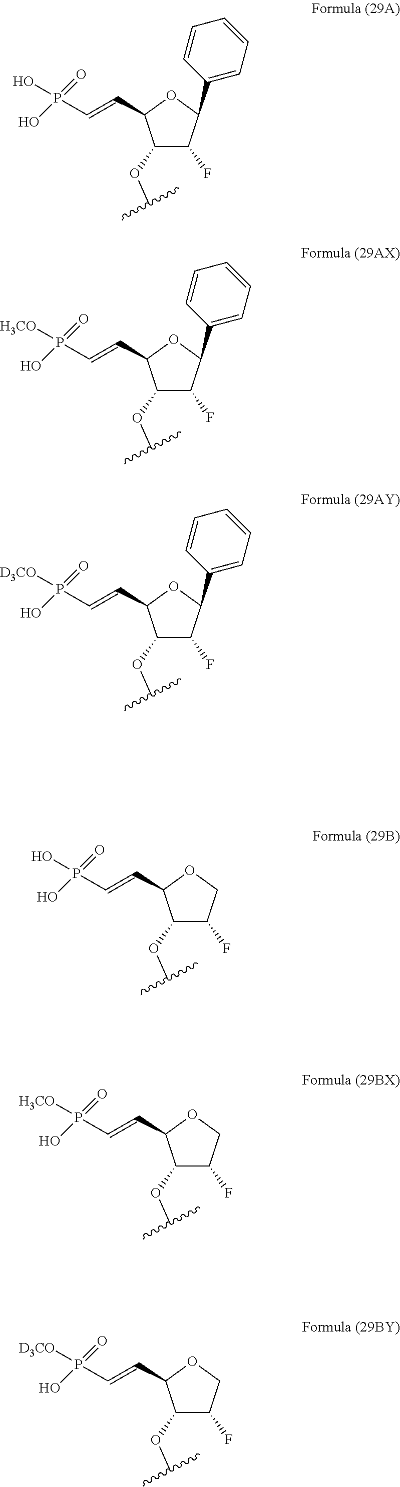

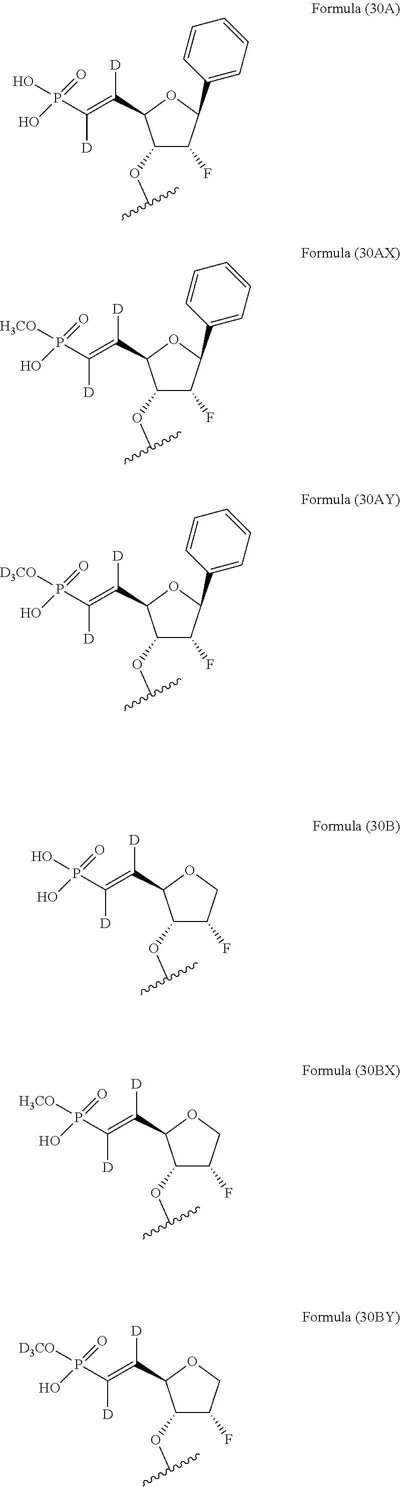

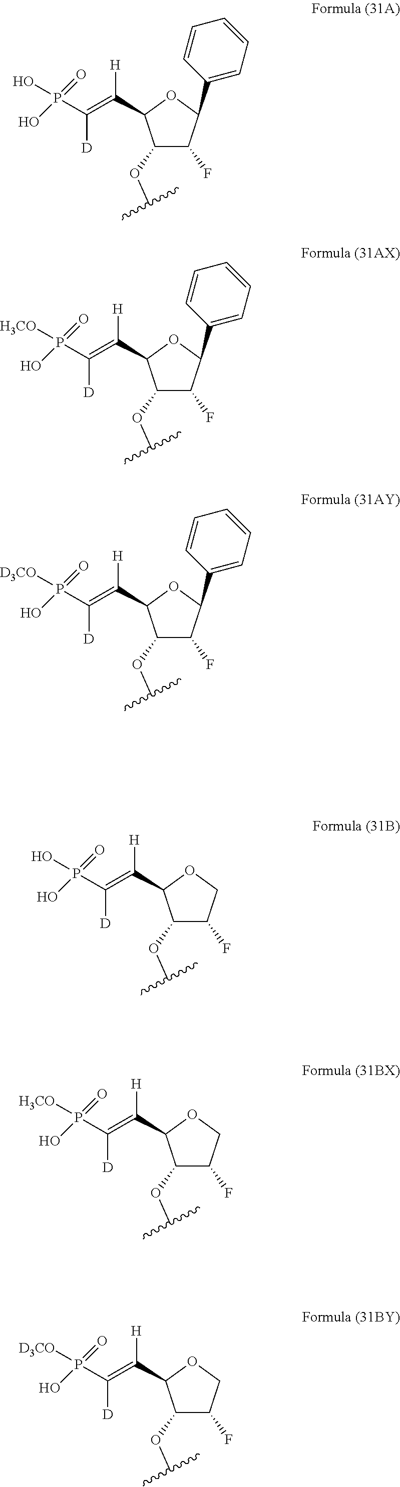

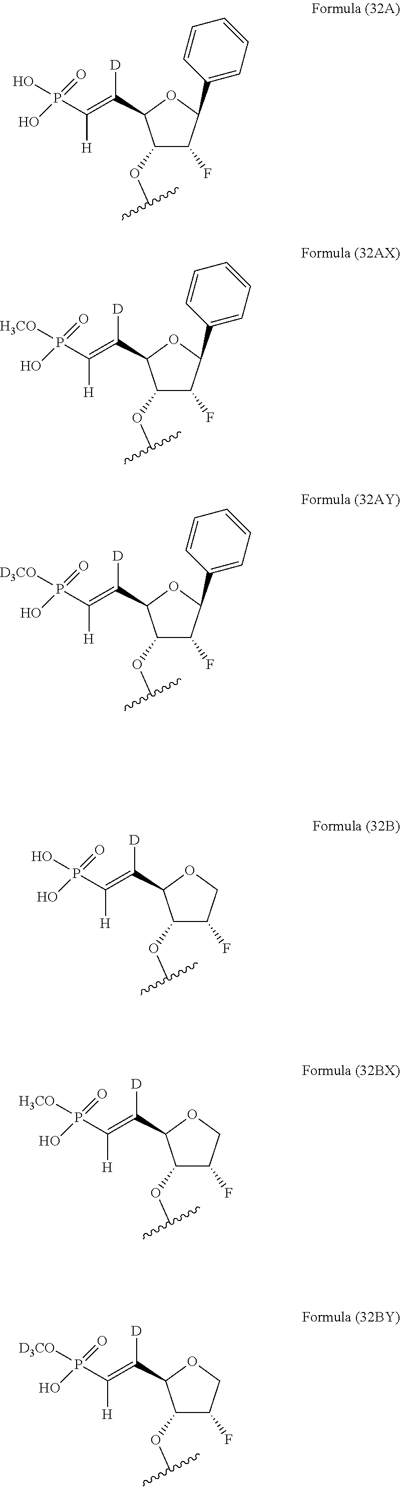

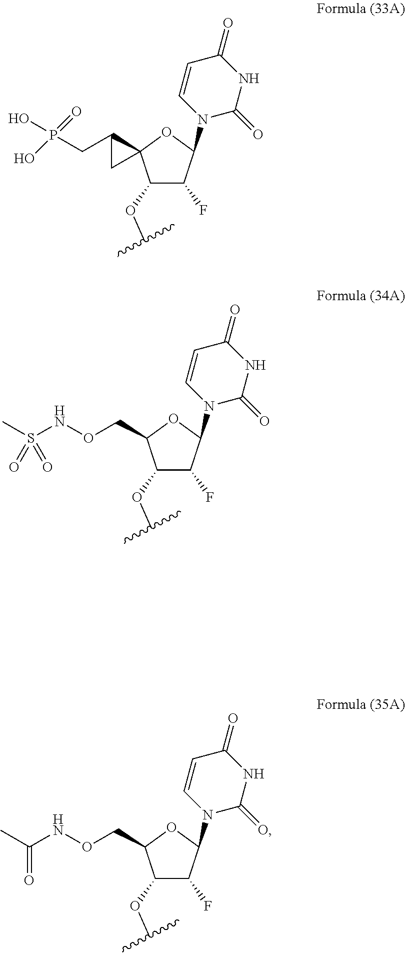

wherein R¹ is a nucleobase, aryl, heteroaryl, or H.

Embodiment 52. In any one of embodiments 49-51, the 5′-stabilized end cap may comprise a 5′ vinyl phosphonate or deuterated 5′ vinyl phosphonate.

Embodiment 53. In embodiments 49 or 50, the 5′-stabilized end cap may be Formula (IA) or (IIB):

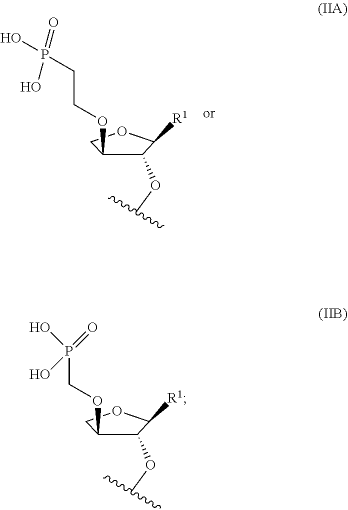

wherein R¹ is a nucleobase, aryl, heteroaryl, or H.

Embodiment 54. In embodiments 49 or 50, the 5′-stabilized end cap may be Formula (IIIA) or (IIIB):

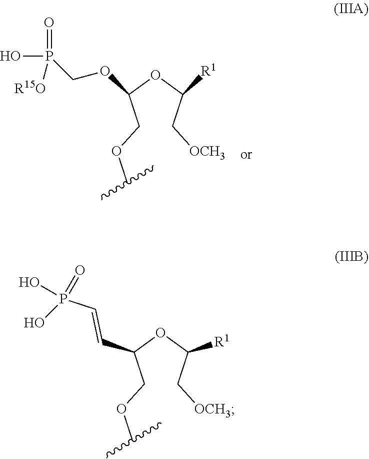

wherein R¹ is a nucleobase, aryl, heteroaryl, or H; and R¹⁵ is H or CH₃.

Embodiment 55. In embodiment 49 or 50, the 5′-stabilized end cap may be

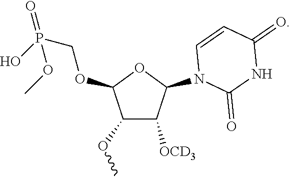

Embodiment 56. In any one of embodiments 49-55, the 5′ end of the antisense strand via one or more linkers independently selected from a phosphodiester linker, phosphorothioate linker, or phosphorodithioate linker.

Embodiment 57. In any one of embodiments 49-56, the 5′ end of the sense strand via one or more linkers independently selected from a phosphodiester linker, phosphorothioate linker, or phosphorodithioate linker.

Embodiment 58. In any proceeding embodiment, the first nucleotide from the 5′ end of the first nucleotide sequence comprises a phosphorylation blocker.

Embodiment 59. In any proceeding embodiment, the first nucleotide from the 5′ end of the second nucleotide sequence comprises a phosphorylation blocker.

Embodiment 60. In embodiment 58 or 59, the phosphorylation blocker may be a phosphorylation blocker of Formula (IV):

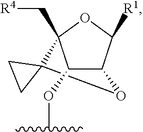

wherein R¹ is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring.

Embodiment 61. In any proceeding embodiment, the siNA molecule may include at least one modified nucleotide of Formula (IV):

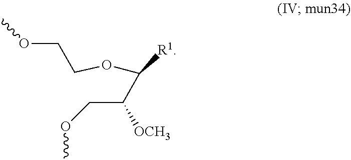

Embodiment 62. In any proceeding embodiment, the phosphorylation blocker is attached to the 5′ end of the sense strand.

Embodiment 63. In any proceeding embodiment, the phosphorylation blocker is attached to the 3′ end of the sense strand.

Embodiment 64. In any proceeding embodiment, the phosphorylation blocker is attached to the 5′ end of the antisense strand.

Embodiment 65. In any proceeding embodiment, the phosphorylation blocker is attached to the 3′ end of the antisense strand.

Embodiment 66. In any proceeding embodiment, the phosphorylation blocker is attached to the 5′ end of the sense strand via one or more linkers independently selected from a phosphodiester linker, phosphorothioate linker, and phosphorodithioate linker.

Embodiment 67. In any proceeding embodiment, the phosphorylation blocker is attached to the 3′ end of the sense strand via one or more linkers independently selected from a phosphodiester linker, phosphorothioate linker, and phosphorodithioate linker.

Embodiment 68. In any proceeding embodiment, the phosphorylation blocker is attached to the 5′ end of the antisense strand via one or more linkers independently selected from a phosphodiester linker, phosphorothioate linker, and phosphorodithioate linker.

Embodiment 69. In any proceeding embodiment, the phosphorylation blocker is attached to the 3′ end of the antisense strand via one or more linkers independently selected from a phosphodiester linker, phosphorothioate linker, and phosphorodithioate linker.

Embodiment 70. In any proceeding embodiment, the siNA molecule further comprises a galactosamine.

Embodiment 71. In embodiment 70, the galactosamine is N-acetylgalactosamine (GalNAc) of Formula (VII):

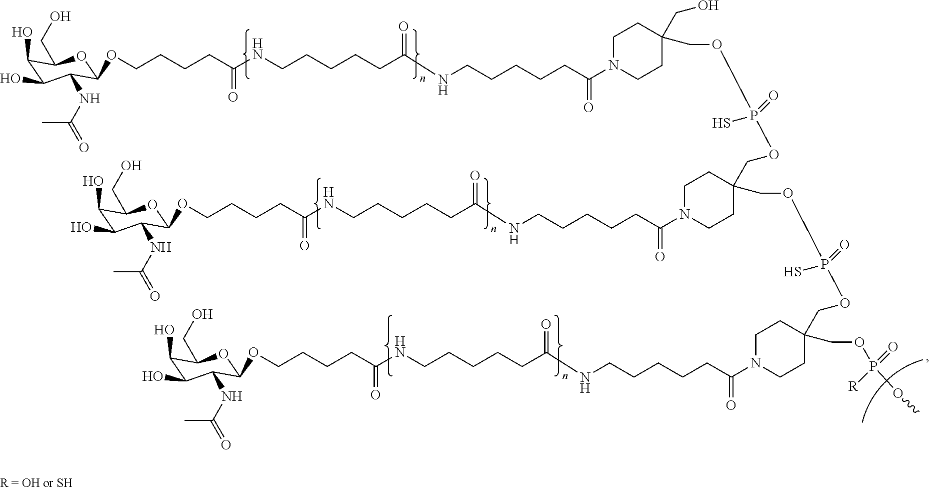

wherein each n is independently 1 or 2.

Embodiment 72. In embodiment 70, the galactosamine is N-acetylgalactosamine (GalNAc) of Formula (VI):

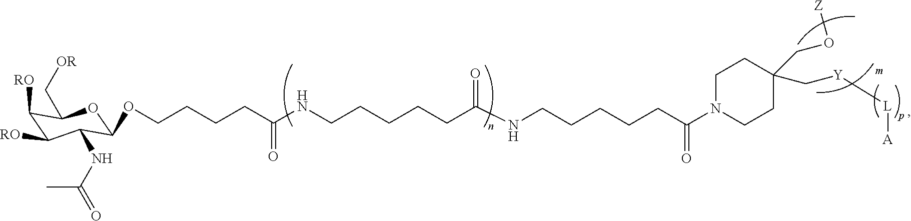

wherein m is 1, 2, 3, 4, or 5; each n is independently 1 or 2; p is 0 or 1; each R is independently H; each Y is independently selected from —O—P(═O)(SH)—, —O—P(═O)(O)—, —O—P(═O)(OH)—, and —O—P(S)S—; Z is H or a second protecting group; either L is a linker or L and Y in combination are a linker; and A is H, OH, a third protecting group, an activated group, or an oligonucleotide. In some embodiments, A is an oligonucleotide. In some embodiments, A is 1-2 oligonucleotides. In some embodiments, the oligonucleotide is dTdT.

Embodiment 73. In any one of embodiments 70-72, the galactosamine may be attached to the 3′ end of the sense strand.

Embodiment 74. In any one of embodiments 70-73, the galactosamine may be attached to the 5′ end of the sense strand.

Embodiment 75. In any one of embodiments 70-74, the galactosamine may be attached to the 3′ end of the antisense strand.

Embodiment 76. In any one of embodiments 70-75, the galactosamine may be attached to the 5′ end of the antisense strand.

Embodiment 77. In any one of embodiments 70-76, the galactosamine may be attached to the 3′ end of the sense strand via one or more linkers independently selected from a phosphodiester linker, phosphorothioate linker, or phosphorodithioate linker.

Embodiment 78. In any one of embodiments 70-77, the galactosamine may be attached to the 5′ end of the sense strand via one or more linkers independently selected from a phosphodiester linker, phosphorothioate linker, or phosphorodithioate linker.

Embodiment 80. In any one of embodiments 70-78, the galactosamine may be attached to the 3′ end of the antisense strand via one or more linkers independently selected from a phosphodiester linker, phosphorothioate linker, or phosphorodithioate linker.

Embodiment 81. In any one of embodiments 70-79, the galactosamine may be attached to the 5′ end of the antisense strand via one or more linkers independently selected from a phosphodiester linker, phosphorothioate linker, or phosphorodithioate linker.

Embodiment 82. In any proceeding embodiment, the antisense strand, sense strand, first nucleotide sequence, and/or second nucleotide sequence comprises at least one thermally destabilizing nucleotide selected from:

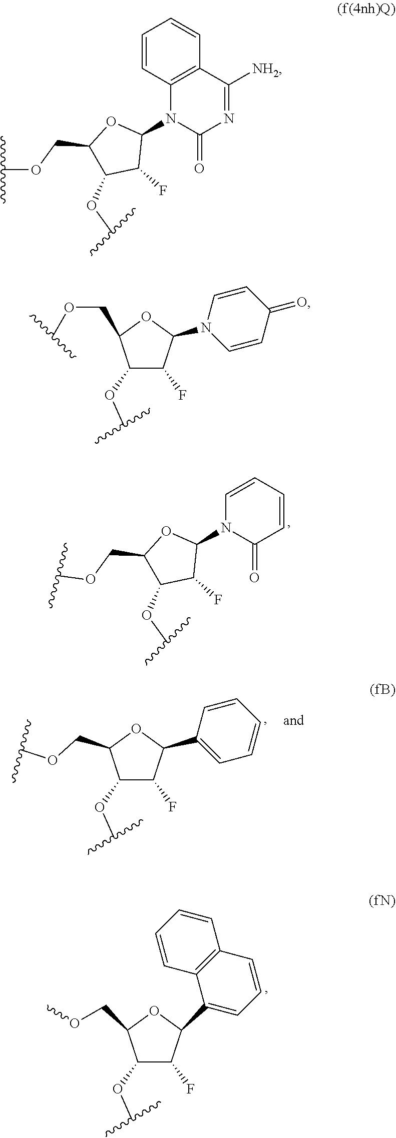

or a combination thereof.

Embodiment 83. In any proceeding embodiment, at least one 2′-fluoro nucleotide or 2′-O-methyl nucleotide is a 2′-fluoro or 2-O-methyl nucleotide mimic of Formula (V):

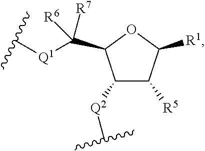

wherein R¹ is independently a nucleobase, aryl, heteroaryl, or H; Q¹ and Q² are independently S or Ol R⁵ is independently —OCD₃, —F, or —OCH₃; and R⁶ and R⁷ are independently H, D, or CD₃.

Embodiment 84. In embodiment 83, the 2′-fluoro or 2′-O-methyl nucleotide mimic may be a nucleotide mimic of Formula (16)-Formula (20):

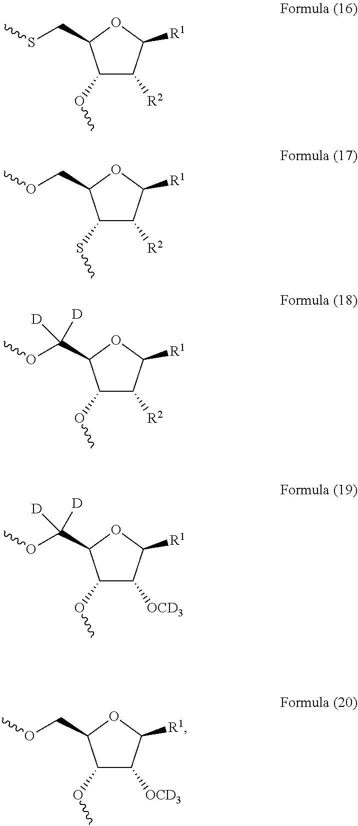

wherein R¹ is a nucleobase and R² is independently F or —OCH₃.

Embodiment 85. In any proceeding embodiment, at least one 2′-fluoro nucleotide may be a 2′-fluoro nucleotide mimic.

Embodiment 86. In embodiment 85, at least 1, 2, 3, 4, 5, or more 2′-fluoro nucleotides on the antisense strand or the second nucleotide sequence is a 2′-fluoro nucleotide mimic.

Embodiment 87. In embodiment 85 or 86, the nucleotide at position 2, 5, 6, 8, 10, 14, 16, and/or 17 from the 5′ end of the antisense strand or the second nucleotide sequence is a 2′-fluoro nucleotide mimic.

Embodiment 88. In any one of embodiments 85-87, at least 1, 2, 3, 4, 5, or more 2′-fluoro nucleotides on the sense strand or the first nucleotide sequence is a 2′-fluoro nucleotide mimic.

Embodiment 89. In any one of embodiments 85-88, the nucleotide at position 3, 5, 7, 8, 9, 10, 11, 12, 14, and/or 17 from the 5′ end of the sense strand or the first nucleotide sequence is a 2′-fluoro nucleotide mimic.

Embodiment 90. In any one of embodiments 85-89, at least 1, 2, 3, 4, 5, 6, or more 2′-fluoro nucleotide mimics is a f4P nucleotide

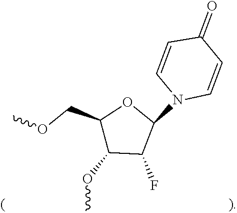

Embodiment 91. In any one of embodiments 85-90, less than or equal to 10, 9, 8, 7, 6, 5, 4, 3, or 2 2′-fluoro nucleotide mimics is a f4P nucleotide

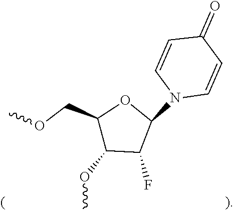

Embodiment 92. In any one of embodiments 85-91, 1, 2, 3, 4, 5, 6, or more 2′-fluoro nucleotide mimics is a f2P nucleotide

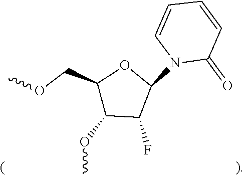

Embodiment 93. In any one of embodiments 85-92, less than or equal to 10, 9, 8, 7, 6, 5, 4, 3, or 2 2′-fluoro nucleotide mimics is a f2P nucleotide

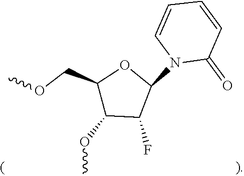

Embodiment 94. In any one of embodiments 85-93, 1, 2, 3, 4, 5, 6, or more 2′-fluoro nucleotide mimics is a fX nucleotide

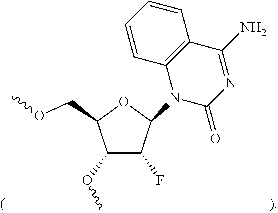

Embodiment 95. In any one of embodiments 85-94, less than or equal to 10, 9, 8, 7, 6, 5, 4, 3, or 2 2′-fluoro nucleotide mimics is a fX nucleotide

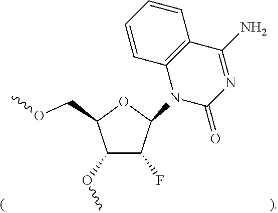

Embodiment 96. In any proceeding embodiment, the first nucleotide from the 5′ end of the antisense strand or second nucleotide sequence is a d2vd3 nucleotide

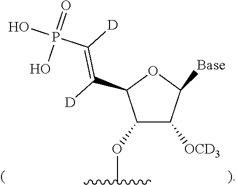

Embodiment 97. In any proceeding embodiment, the first nucleotide from the 3′ end of the antisense strand or second nucleotide sequence is a d2vd3 nucleotide

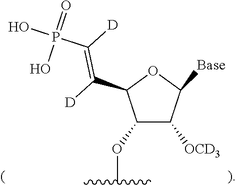

Embodiment 98. In any proceeding embodiment, at least one end of the siNA is a blunt end.

Embodiment 99. In any proceeding embodiment, at least one end of the siNA comprises an overhang, wherein the overhang comprises at least one nucleotide.

Embodiment 100. In any proceeding embodiment, both ends of the siNA comprise an overhang, wherein the overhang comprises at least one nucleotide.

Embodiment 101. In any proceeding embodiment, the target gene is a hydroxysteroid 17-beta dehydrogenase 13 (HSD17B13).

Embodiment 102. In any proceeding embodiment, the first nucleotide sequence comprises a nucleotide sequence of any one of SEQ ID Nos: 1-100, 201-230, 262-287, 314, or 315 and the second nucleotide sequence comprises a nucleotide sequence of any one of SEQ ID NOs: 101-200, 231-260, or 288-313.

Embodiment 103. A composition comprising the siNA molecule according to any preceding embodiment and a pharmaceutically acceptable excipient.

Embodiment 104. In embodiment 103, further comprising an additional liver disease treatment agent.

Embodiment 105. In embodiment 103 or 104, further comprising an additional siNA.

Embodiment 106. In any one of embodiments 103-105, further comprising a liver disease treatment agent. In some embodiments, the liver disease treatment agent may be selected from a peroxisome proliferator-activator receptor (PPAR) agonist, farnesoid X receptor (FXR) agonist, lipid-altering agent, and incretin-based therapy. In some embodiments, the liver disease treatment agent may be a PPAR agonist is selected from a PPARα agonist, dual PPARα/δ agonist, PPARγ agonist, and dual PPARα/γ agonist. In some embodiments, the liver disease treatment agent may be selected from a fibrate, elafibranor, thiazolidinedione (TZD), pioglitazone, saroglitazar, obeticholic acis (OCA), aramchol, glucagon-like peptide 1 (GLP-1) receptor agonist, dipeptidyl peptidase 4 (DPP-4) inhibitor, exenatide, liraglutide sitagliptin, and vildapliptin.

Embodiment 107. The composition according to any one of embodiments 103-106 for use as a medicament.

Embodiment 108. Use of the siNA molecule or the composition according to any preceding embodiment in the manufacture of a medicament for treating a disease.

Embodiment 109. Use of the siNA molecule or the composition according to any preceding embodiment in the treatment of a disease.

Embodiment 110. The siNA molecule according to any one of embodiments 1-102 for use as a medicament.

Embodiment 111. The siNA molecule according to any one of embodiments 1-102 for use in the treatment of a disease.

Embodiment 112. In any of embodiments 109-111, wherein the disease is a liver disease.

Embodiment 113. In any of embodiments 109-112, wherein the disease is a nonalcoholic fatty liver disease (NAFLD) or hepatocellular carcinoma (HCC).

Embodiment 114. In any of embodiments 109-113, wherein the disease is nonalcoholic steatohepatitis (NASH).

Embodiment 115. A short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and
(b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide;
      wherein the first nucleotide sequence comprises a nucleotide sequence of any one of SEQ ID Nos: 1-100, 201-230, 262-287, 314, or 315, the second nucleotide sequence comprises a nucleotide sequence of any one of SEQ ID NOs: 101-200, 231-260, or 288-313, or a combination thereof.

Embodiment 116. A short interfering nucleic acid (siNA) molecule comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide; and
(b) an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide;
      wherein the siNA reduces or inhibits the production of a hydroxysteroid dehydrogenase.

Embodiment 117. The siNA molecule according to Embodiment 115 or Embodiment 116, wherein the first nucleotide sequence comprises 16, 17, 18, 19, 20, 21, 22, 23, or more modified nucleotides independently selected from a 2′-O-methyl nucleotide and a 2′-fluoro nucleotide;

optionally wherein:
70%, 75%, 80%, 85%, 90%, 95% or 100% of the nucleotides in the first nucleotide sequence are modified nucleotides independently selected from a 2′-O-methyl nucleotide and a 2′-fluoro nucleotide;
at least 2, 3, 4, 5, or 6 modified nucleotides of the first nucleotide sequence are 2′-fluoro nucleotides;
no more than 10, 9, 8, 7, 6, 5, 4, 3, or 2 modified nucleotides of the first nucleotide sequence are 2′-fluoro nucleotides;
at least 10, 11, 12, 13, 14, 15, 16, 17, 18, 19, 20, 21, or 22 modified nucleotides of the first nucleotide sequence are 2′-O-methyl nucleotides; and/or
no more than 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides.

Embodiment 118. The siNA molecule according to any one of Embodiments 115-117, wherein the second nucleotide sequence comprises 16, 17, 18, 19, 20, 21, 22, 23, or more modified nucleotides independently selected from a 2′-O-methyl nucleotide and a 2′-fluoro nucleotide; optionally wherein:
70%, 75%, 80%, 85%, 90%, 95% or 100% of the nucleotides in the second nucleotide sequence are modified nucleotides independently selected from a 2′-O-methyl nucleotide and a 2′-fluoro nucleotide;
at least 2, 3, 4, 5, or 6 modified nucleotides of the second nucleotide sequence are 2′-fluoro nucleotides;
less than or equal to 10, 9, 8, 7, 6, 5, 4, 3, or 2 modified nucleotides of the second nucleotide sequence are 2′-fluoro nucleotides;
at least 10, 11, 12, 13, 14, 15, 16, 17, 18, 19, 20, 21, or 22 modified nucleotides of the second nucleotide sequence are 2′-O-methyl nucleotides; and/or
less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides.

Embodiment 119. The siNA molecule according to any one of Embodiments 115-118, wherein at least:
1, 2, 3, 4, 5, 6, or 7 nucleotides at position 3, 5, 7, 8, 9, 10, 11, 12, and/or 17 from the 5′ end of the first nucleotide sequence is a 2′-fluoro nucleotide; and/or
1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide.

Embodiment 120. The siNA molecule according to any one of Embodiments 115-119, wherein:
(i) the nucleotides in the second nucleotide sequence are arranged in an alternating 1:3 modification pattern, and
(ii) 1 nucleotide is a 2′-fluoro nucleotide and 3 nucleotides are 2′-O-methyl nucleotides; optionally wherein the alternating 1:3 modification pattern occurs 2-5 times.

Embodiment 121. The siNA molecule according to any one of Embodiments 115-119, wherein:
(i) the nucleotides in the second nucleotide sequence are arranged in an alternating 1:2 modification pattern, and
(ii) 1 nucleotide is a 2′-fluoro nucleotide and 2 nucleotides are 2′-O-methyl nucleotides; optionally wherein the alternating 1:2 modification pattern occurs 2-5 times.

Embodiment 122. A short interfering nucleic acid (siNA) molecule represented by Formula (VIII):

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′-stabilized end cap or a phosphorylation blocker;
B is a 2′-fluoro nucleotide;
C represents overhanging nucleotides and is a 2′-O-methyl nucleotide, deoxy nucleotide, or uracil;
n¹=1-6 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length;
wherein the first nucleotide sequence comprises a nucleotide sequence of any one of SEQ ID Nos: 1-100, 201-230, 262-287, 314, or 315, the second nucleotide sequence comprises a nucleotide sequence of any one of SEQ ID NOs: 101-200, 231-260, or 288-313, or any combination thereof.

Embodiment 123. A short interfering nucleic acid (siNA) molecule represented by Formula (VIII):

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′-stabilized end cap or a phosphorylation blocker;
B is a 2′-fluoro nucleotide;
C represents overhanging nucleotides and is a 2′-O-methyl nucleotide, deoxy nucleotide, or uracil;
n¹=1-6 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length;
   wherein the siNA reduces or inhibits the production of a hydroxysteroid dehydrogenase

Embodiment 124. The siNA molecule according to Embodiment 123 represented by Formula (IX):

5′-A₂₋₆B₁A₁₋₃B₂₋₃A₂₋₁₀B₀₋₁A₀₋₄B₀₋₁A₀₋₂-3′
3′-C₂A₀₋₂B₀₋₁A₀₋₃B₀₋₁A₀₋₅B₀₋₁A₂₋₇B₁A₂₋₁₁B₁A₁-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′-stabilized end cap or a phosphorylation blocker;
B is a 2′-fluoro nucleotide;
C represents overhanging nucleotides and is a 2′-O-methyl nucleotide, deoxy nucleotide, or uracil.

Embodiment 125. The siNA molecule according to Embodiment 123 or Embodiment 124
a. a sense strand comprising a first nucleotide sequence consisting of 17 to 23 nucleotides, wherein 2′-fluoro nucleotides are at positions 3, 7-9, 12, and 17 from the 5′ end of the first nucleotide sequence, and wherein 2′-O-methyl nucleotides are at positions 1, 2, 4-6, 10, 11, and 13-16 from the 5′ end of the first nucleotide sequence; and
b. an antisense strand comprising a second nucleotide sequence consisting of 17 to 23 nucleotides, wherein:
   2′-fluoro nucleotides are at positions 2 and 14 from the 5′ end of the second nucleotide sequence, and wherein 2′-O-methyl nucleotides are at positions 1, 3-13, and 15-17 from the 5′ end of the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 19 nucleotides;
2′-O-methyl nucleotides are at positions 18 and 19 from the 5′ end of the first nucleotide sequence;
the second nucleotide sequence consists of 21 nucleotides; and/or
2′-O-methyl nucleotides are at positions 18-21 or 19-21 from the 5′ end of the second nucleotide sequence.

Embodiment 126. The siNA molecule according to Embodiment 123 or Embodiment 124
c. a sense strand comprising a first nucleotide sequence consisting of 17 to 23 nucleotides, wherein 2′-fluoro nucleotides are at positions 3, 7, 8, and 17 from the 5′ end of the first nucleotide sequence, and wherein 2′-O-methyl nucleotides are at positions 1, 2, 4-6, and 9-16 from the 5′ end of the first nucleotide sequence; and
d. an antisense strand comprising a second nucleotide sequence consisting of 17 to 23 nucleotides, wherein:
   2′-fluoro nucleotides are at positions 2 and 14 from the 5′ end of the second nucleotide sequence, and wherein 2′-O-methyl nucleotides are at positions 1, 3-13, and 15-17 from the 5′ end of the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 19 nucleotides;
2′-O-methyl nucleotides are at positions 18 and 19 from the 5′ end of the first nucleotide sequence;
the second nucleotide sequence consists of 21 nucleotides; and/or
2′-O-methyl nucleotides are at positions 18-21 or 19-21 from the 5′ end of the second nucleotide sequence.

Embodiment 127. The siNA molecule according to Embodiment 123 or Embodiment 124
e. a sense strand comprising a first nucleotide sequence consisting of 17 to 23 nucleotides, wherein 2′-fluoro nucleotides are at positions 3, 7-9, and 17 from the 5′ end of the first nucleotide sequence, and wherein 2′-O-methyl nucleotides are at positions 1, 2, 4-6, and 10-16 from the 5′ end of the first nucleotide sequence; and
f. an antisense strand comprising a second nucleotide sequence consisting of 17 to 23 nucleotides, wherein:
   2′-fluoro nucleotides are at positions 2, 6, 10, and 14 from the 5′ end of the second nucleotide sequence, and wherein 2′-O-methyl nucleotides are at positions 1, 3-5, 7-9, 11-13, and 15-17 from the 5′ end of the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 19 nucleotides;
2′-O-methyl nucleotides are at positions 18 and 19 from the 5′ end of the first nucleotide sequence;
the second nucleotide sequence consists of 21 nucleotides; and/or
2′-O-methyl nucleotides are at positions 19-21 from the 5′ end of the second nucleotide sequence.

Embodiment 128. The siNA molecule according to Embodiment 123 or Embodiment 124
g. a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O-methyl nucleotides are at positions 1-4, 6, and 10-17 from the 5′ end of the first nucleotide sequence; and
h. an antisense strand comprising a second nucleotide sequence consisting of 17 to 23 nucleotides, wherein:
   2′-fluoro nucleotides are at positions 2, 6, 10, and 14 from the 5′ end of the second nucleotide sequence, and wherein 2′-O-methyl nucleotides are at positions 1, 3-5, 7-9, 11-13 and 15-17 from the 5′ end of the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 19 nucleotides;
2′-O-methyl nucleotides are at positions 18 and 19 from the 5′ end of the first nucleotide sequence;
the second nucleotide sequence consists of 21 nucleotides;
2′-fluoro nucleotide is at position 18 from the 5′ end of the second nucleotide sequence; and/or
2′-O-methyl nucleotides are at positions 19-21 from the 5′ end of the second nucleotide sequence.

Embodiment 129. The siNA molecule according to Embodiment 123 or Embodiment 124
i. a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O-methyl nucleotides are at positions 1-4, 6, and 10-17 from the 5′ end of the first nucleotide sequence; and
j. an antisense strand comprising a second nucleotide sequence consisting of 17 to 23 nucleotides, wherein:
   2′-fluoro nucleotides are at positions 2, 5, 8, 14, and 17 from the 5′ end of the first nucleotide sequence, and wherein 2′-O-methyl nucleotides are at positions 1, 3, 4, 6, 7, 9-13, 15, and 16 from the 5′ end of the first nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 19 nucleotides;
2′-O-methyl nucleotides are at positions 18 and 19 from the 5′ end of the first nucleotide sequence;
the second nucleotide sequence consists of 21 nucleotides; and/or
2′-O-methyl nucleotides are at positions 18-21 or 19-21 from the 5′ end of the second nucleotide sequence.

Embodiment 130. The siNA molecule according to Embodiment 123 or Embodiment 124
k. a sense strand comprising a first nucleotide sequence consisting of 17 to 23 nucleotides, wherein 2′-fluoro nucleotides are at positions 5 and 7-9 from the 5′ end of the first nucleotide sequence, and wherein 2′-O-methyl nucleotides are at positions 1-4, 6, and 10-17 from the 5′ end of the first nucleotide sequence; and
l. an antisense strand comprising a second nucleotide sequence consisting of 17 to 23 nucleotides, wherein:
   2′-fluoro nucleotides are at positions 2, 6, 14, and 16 from the 5′ end of the second nucleotide sequence, and wherein 2′-O-methyl nucleotides are at positions 1, 3-5, 7-13, 15, and 17 from the 5′ end the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
first nucleotide sequence consists of 19 nucleotides;
2′-O-methyl nucleotides are at positions 18 and 19 from the 5′ end of the first nucleotide sequence;
the second nucleotide sequence consists of 21 nucleotides; and/or
2′-O-methyl nucleotides are at positions 18-21 or 19-21 from the 5′ end of the second nucleotide sequence.

Embodiment 131. The siNA molecule according to Embodiment 123 or Embodiment 124
m. a sense strand comprising a first nucleotide sequence consisting of 17 to 23 nucleotides, wherein 2′-fluoro nucleotides are at positions 5, 9-11, and 14 from the 5′ end of the first nucleotide sequence, and wherein 2′-O-methyl nucleotides are at positions 1-4, 6-8, 12, 13, and 15-17 from the 5′ end of the first nucleotide sequence; and
n. an antisense strand comprising a second nucleotide sequence consisting of 17 to 23 nucleotides, wherein:
   2′-fluoro nucleotides are at positions 2 and 14 from the 5′ end of the second nucleotide sequence, and wherein 2′-O-methyl nucleotides are at positions 1, 3-13, and 15-17 from the 5′ end the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 21 nucleotides;
2′-O-methyl nucleotides are at positions 18, 20, and 21 from the 5′ end of the first nucleotide sequence;
2′-fluoro nucleotide is at position 19 from the 5′ end of the first nucleotide sequence; the second nucleotide sequence consists of 23 nucleotides; and/or
2′-O-methyl nucleotides are at positions 18-23 from the 5′ end of the second nucleotide sequence.

Embodiment 132. The siNA molecule according to Embodiment 123 or Embodiment 124
o. a sense strand comprising a first nucleotide sequence consisting of 17 to 23 nucleotides, wherein 2′-fluoro nucleotides are at positions 7 and 9-11 from the 5′ end of the first nucleotide sequence, and wherein 2′-O-methyl nucleotides are at positions 1-6, 8, and 12-17 from the 5′ end of the first nucleotide sequence; and
p. an antisense strand comprising a second nucleotide sequence consisting of 17 to 23 nucleotides, wherein:
   2′-fluoro nucleotides are at positions 2, 6, 14, and 16 from the 5′ end of the second nucleotide sequence, and wherein 2′-O-methyl nucleotides are at positions 1, 3-5, 7-13, 15, and 17 from the 5′ end the second nucleotide sequence; or
   the nucleotides in the second nucleotide sequence are arranged in an alternating 1:3 modification pattern, and wherein 1 nucleotide is a 2′-fluoro nucleotide and 3 nucleotides are 2′-O-methyl nucleotides; or
   the nucleotides in the second nucleotide sequence are arranged in an alternating 1:2 modification pattern, and wherein 1 nucleotide is a 2′-fluoro nucleotide and 2 nucleotides are 2′-O-methyl nucleotides;
      optionally wherein:
the first nucleotide sequence consists of 21 nucleotides;
2′-O-methyl nucleotides are at positions 18-21 from the 5′ end of the first nucleotide sequence;
the second nucleotide sequence consists of 23 nucleotides; and/or
2′-O-methyl nucleotides are at positions 18-21 from the 5′ end of the second nucleotide sequence.

Embodiment 133. The siNA molecule according to any preceding Embodiment, wherein the sense strand further comprises TT sequence adjacent to the first nucleotide sequence, the antisense strand further comprises TT sequence adjacent to the second nucleotide sequence, or a combination thereof.

Embodiment 134. The siNA molecule according to any preceding Embodiment, wherein the first nucleotide sequence further comprises 1 or more phosphorothioate internucleoside linkages, the second nucleotide sequence further comprises 1 or more phosphorothioate internucleoside linkages, or a combination thereof;

optionally wherein:
the sense strand comprises at least 1, 2, 3, 4, 5, 6, 7, 8, 9, 10, 11, 12, 13, 14, 15 or more phosphorothioate internucleoside linkages;
the antisense strand further comprises at least 1, 2, 3, 4, 5, 6, 7, 8, 9, 10, 11, 12, 13, 14, 15 or more phosphorothioate internucleoside linkages;

Embodiment 135. The siNA molecule of Embodiment 134, wherein:
(i) at least one phosphorothioate internucleoside linkage is between the nucleotides at positions 1 and 2 from the 5′ end of the first nucleotide sequence and/or second nucleotide sequence; and/or
(ii) at least one phosphorothioate internucleoside linkage is between the nucleotides at positions 2 and 3 from the 5′ end of the first nucleotide sequence and/or second nucleotide sequence; and/or
(iii) at least one phosphorothioate internucleoside linkage is between the nucleotides at positions 1 and 2 from the 3′ end of the first nucleotide sequence and/or second nucleotide sequence; and/or
(iv) at least one phosphorothioate internucleoside linkage is between the nucleotides at positions 2 and 3 from the 3′ end of the first nucleotide sequence and/or second nucleotide sequence.

Embodiment 136. The siNA molecule according to any preceding Embodiment, wherein the first nucleotide sequence further comprises 1 or more mesyl phosphoroamidate internucleoside linkages, the second nucleotide sequence further comprises 1 or more mesyl phosphoroamidate internucleoside linkages, or a combination thereof;

optionally wherein:
the sense strand comprises at least 1, 2, 3, 4, 5, 6, 7, 8, 9, 10, 11, 12, 13, 14, 15 or more mesyl phosphoroamidate internucleoside linkages;
the antisense strand further comprises at least 1, 2, 3, 4, 5, 6, 7, 8, 9, 10, 11, 12, 13, 14, 15 or more mesyl phosphoroamidate internucleoside linkages;

Embodiment 137. The siNA molecule of Embodiment 136, wherein:
(i) at least one mesyl phosphoroamidate internucleoside linkage is between the nucleotides at positions 1 and 2 from the 5′ end of the first nucleotide sequence and/or second nucleotide sequence;
(ii) at least one mesyl phosphoroamidate internucleoside linkage is between the nucleotides at positions 2 and 3 from the 5′ end of the first nucleotide sequence and/or second nucleotide sequence;
(iii) at least one mesyl phosphoroamidate internucleoside linkage is between the nucleotides at positions 1 and 2 from the 3′ end of the first nucleotide sequence and/or second nucleotide sequence; and/or
(iv) at least one mesyl phosphoroamidate internucleoside linkage is between the nucleotides at positions 2 and 3 from the 3′ end of the first nucleotide sequence and/or second nucleotide sequence.

Embodiment 138. The siNA molecule according to any preceding Embodiment, wherein the sense strand further comprises a stabilizing end cap, antisense strand further comprises a stabilizing end cap, or a combination thereof.

Embodiment 139. The siNA molecule according to Embodiment 138 comprising a 5′-stabilized end cap of Formula (I′):

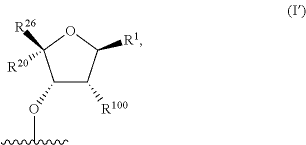

wherein:
R¹ is a nucleobase, aryl, heteroaryl, or H,
R²⁶ is

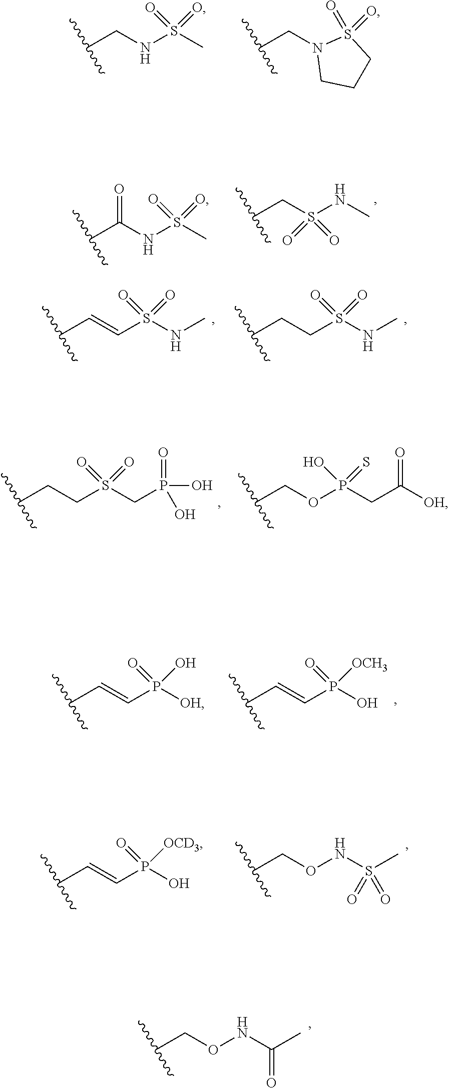

—CH═CD-Z, —CD═CH—Z, —CD═CD-Z, —(CR²¹R²²)ₙ—Z, or —(C₂-C₆ alkenylene)-Z and R²⁰ is hydrogen; or
R²⁶ and R²⁰ together form a 3- to 7-membered carbocyclic ring substituted with —(CR²¹R²²)ₙ—Z or —(C₂-C₆ alkenylene)-Z;
n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂ (CH₂)ₘP(O)(OH)₂, —SO₂NR²³R²⁵, —NR²³R²⁴,
R²¹ and R²² are independently hydrogen or C₁-C₆ alkyl; R²¹ and R²² together form an oxo group;
R²³ is hydrogen or C₁-C₆ alkyl;
R²⁴ is-SO₂R²⁵ or —C(O)R²⁵; or
R²³ and R²⁴ together with the nitrogen to which they are attached form a substituted or unsubstituted heterocyclic ring;
R²⁵ is C₁-C₆ alkyl;
R¹⁰⁰ is —OCH₃, —OCD₃, or —F; and
m is 1, 2, 3, or 4.

Embodiment 140. The siNA molecule according to Embodiment 138 or Embodiment 139, wherein the 5′-stabilized end cap comprises a 5′ vinyl phosphonate or deuterated 5′ vinyl phosphonate.

Embodiment 141. The siNA molecule according to Embodiment 138 comprising a 5′-stabilized end cap of Formula (IIA) or (IIB):

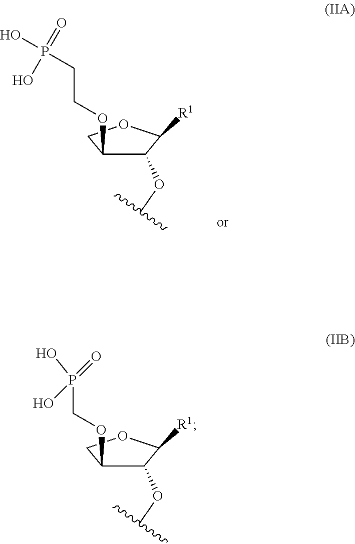

wherein R¹ is a nucleobase, aryl, heteroaryl, or H.

Embodiment 142. The siNA molecule according to Embodiment 135 comprising a 5′-stabilized end cap of Formula (IIIA) or (IIIB):

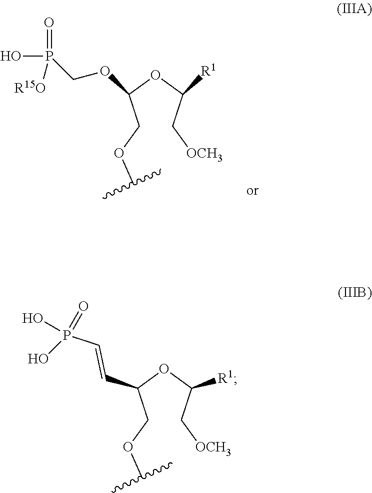

wherein R¹ is a nucleobase, aryl, heteroaryl, or H; and R¹⁵ is H or CH₃.

Embodiment 143. The siNA molecule according to any preceding Embodiment, wherein the sense strand further comprises a phosphorylation blocker, antisense strand further comprises a phosphorylation blocker, or a combination thereof.

Embodiment 144. The siNA molecule according to Embodiment 143, wherein the phosphorylation blocker is a phosphorylation blocker of Formula (IV):

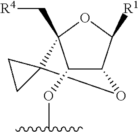

wherein:
R¹ is a nucleobase,
R⁴ is —O—R³⁰ or —NR³¹R³²,
R³⁰ is C₁-C₈ substituted or unsubstituted alkyl; and
R³¹ and R³² together with the nitrogen to which they are attached form a substituted or unsubstituted heterocyclic ring.

Embodiment 145. The siNA molecule according to any preceding Embodiment, comprising at least one modified nucleotide of Formula (IV):

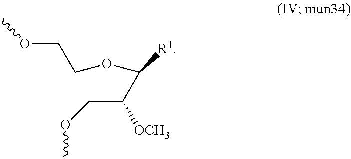

Embodiment 146. The siNA molecule according to any preceding Embodiment, wherein the siNA further comprises a galactosamine.

Embodiment 147. The siNA molecule of Embodiment 146, wherein the galactosamine is N-acetylgalactosamine (GalNAc) of Formula (VI) or Formula (VII):

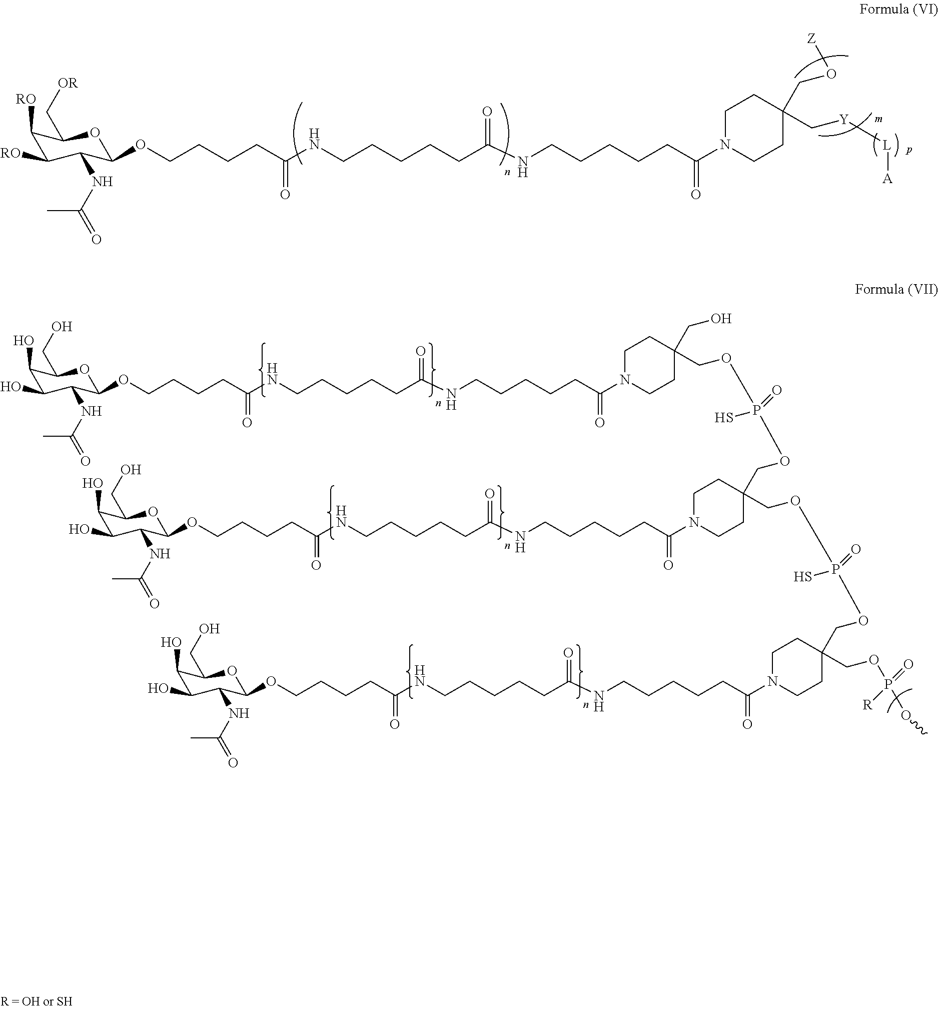

wherein:
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

Embodiment 148. The siNA molecule according to any preceding Embodiment, wherein the antisense strand, sense strand, first nucleotide sequence, and/or second nucleotide sequence comprises at least one thermally destabilizing nucleotide selected from:

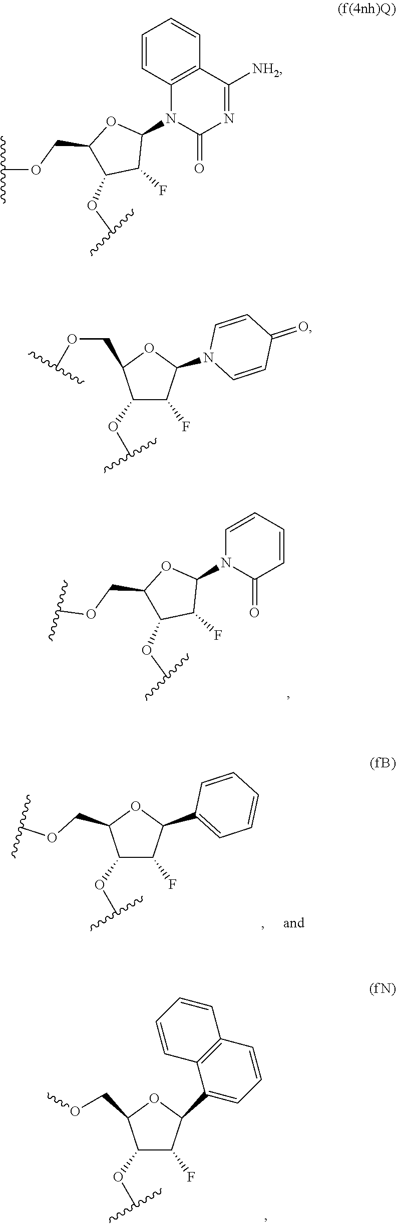

or a combination thereof.

Embodiment 149. The siNA molecule according to any preceding Embodiment, wherein at least one 2′-fluoro nucleotide or 2′-O-methyl nucleotide is a 2′-fluoro or 2-O-methyl nucleotide mimic of Formula (V):

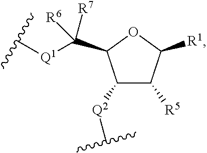

wherein
R¹ is independently a nucleobase, aryl, heteroaryl, or H,
Q¹ and Q² are independently S or O,
R⁵ is independently —OCD₃, —F, or —OCH₃, and
R⁶ and R⁷ are independently H, D, or CD₃.

Embodiment 150. The siNA molecule according to any preceding Embodiment, wherein:
(i) at least one end of the siNA is a blunt end;
(ii) at least one end of the siNA comprises an overhang, wherein the overhang comprises at least one nucleotide; or
(iii) both ends of the siNA comprise an overhang, wherein the overhang comprises at least one nucleotide.

Embodiment 151. The siNA molecule according to any preceding Embodiment, wherein the target gene is a hydroxysteroid 17-beta dehydrogenase 13 (HSD17B13).

Embodiment 152. The siNA molecule according to any preceding Embodiment, wherein the first nucleotide sequence comprises a nucleotide sequence of any one of SEQ ID Nos: 1-100, 201-230, 262-287, 314, or 315 and the second nucleotide sequence comprises a nucleotide sequence of any one of SEQ ID NOs: 101-200, 231-260, or 288-313.

Embodiment 153. A composition comprising the siNA molecule according to any preceding Embodiment and a pharmaceutically acceptable excipient.

Embodiment 154. The composition according to Embodiment 153 further comprising:
(i) an additional liver disease treatment agent; and/or
(ii) an additional siNA.

Embodiment 155. Use of the siNA molecule or the composition according to any preceding Embodiment in the manufacture of a medicament for treating a disease.

Embodiment 156. The use of Embodiment 155, wherein:
(i) the disease is a liver disease;
(ii) the disease is a nonalcoholic fatty liver disease (NAFLD) or hepatocellular carcinoma (HCC); or
(iii) the disease is nonalcoholic steatohepatitis (NASH).

Embodiment 157. The use of Embodiment 155 or Embodiment 156, wherein the disease is nonalcoholic steatohepatitis (NASH).

Embodiment 158. The siNA according to any one of Embodiments 115-152 for use in the treatment of a disease.

Embodiment 159. The siNA of Embodiment 158, wherein:
(i) the disease is a liver disease;
(ii) the disease is a nonalcoholic fatty liver disease (NAFLD) or hepatocellular carcinoma (HCC); or
(iii) the disease is nonalcoholic steatohepatitis (NASH).

1. A double-stranded short interfering nucleic acid (siNA) molecule comprising:
(a) a sense strand comprising a nucleotide sequence that is at least about 60%, 65%, 70%, 75%, 80%, 85%, 90%, 95%, or 100% identical to a nucleotide sequence of any one of SEQ ID NOs: 1-100, 201-230, 262-287, 314-445, 576-603 or 638; and/or
(b) an antisense strand comprising a nucleotide sequence that is at least about 60%, 65%, 70%, 75%, 80%, 85%, 90%, 95%, or 100% identical to a nucleotide sequence of any one of SEQ ID NOs: 101-200, 231-260, 288-313, 446-575, 604-637 or 639-644,
wherein the siNA molecule downregulates expression of a hydroxysteroid 17-beta dehydrogenase 13 (HSD17B13) gene.

2. (canceled)

3. (canceled)

4. A double-stranded short interfering nucleic acid (siNA) molecule comprising:
(a) a sense strand comprising a nucleotide sequence of any one of SEQ ID NOs: 1-100, 201-230, 262-287, 314-445, 576-603 or 638 and/or
(b) an antisense strand comprising a nucleotide sequence of any one of SEQ ID NOs: 101-200, 231-260, 288-313, 446-575, 604-637 or 639-644,
wherein the siNA molecule downregulates expression of a hydroxysteroid 17-beta dehydrogenase 13 (HSD17B13) gene.

5. (canceled)

6. (canceled)

7. The siNA molecule according to claim 4, wherein the sense strand and/or the antisense strand comprises at least one modified nucleotide selected from the group consisting of 2′-O-methyl, a 2′-fluoro, a locked nucleic acid, a nucleoside analog, a 5′ terminal vinyl phosphonate, and a 5′ phosphorothioate internucleoside linkage.

8.-12. (canceled)

13. The siNA molecule according to claim 4, wherein the siNA molecule is selected from any one of siNA Duplex ID Nos. D1-D178 or MD1-MD178.

14. (canceled)

15. (canceled)

16. A pharmaceutical composition comprising the siNA molecule according to claim 4.

17. (canceled)

18. The pharmaceutical composition according to claim 16, further comprising at least one additional active agent, wherein the at least one additional active agent is a liver disease treatment agent.

19. The pharmaceutical composition of claim 18, wherein the liver disease treatment agent is selected from a peroxisome proliferator-activator receptor (PPAR) agonist, farnesoid X receptor (FXR) agonist, lipid-altering agent, incretin-based therapy, and thyroid hormone receptor (THR) modulator.

20.-34. (canceled)

35. A method of treating a liver disease in a subject in need thereof, comprising administering to the subject an amount of the siNA molecule according to claim 4.

36. A method of treating a liver disease in a subject in need thereof, comprising administering to the subject an amount of the pharmaceutical composition according to claim 16.

37. The method of claim 35, wherein the liver disease is a nonalcoholic fatty liver disease (NAFLD).

38. The method of claim 35, wherein the liver disease is nonalcoholic steatohepatitis (NASH).

39. The method according to claim 35, further comprising administering to the subject at least one additional active agent, wherein the at least one additional active agent is a liver disease treatment agent.

40.-103. (canceled)

104. A method of reducing the expression level of HSD17B13 in a subject in need thereof comprising administering to the subject an amount of the siNA molecule according to claim 4, thereby reducing the expression level of HSD17B13 in the subject.

105. A method of preventing at least one symptom of a liver disease in a subject in need thereof comprising administering to the subject an amount of the siNA molecule according to claim 4, thereby preventing at least one symptom of a liver disease in the subject.

106. The siNA molecule according to claim 4, further comprising a ligand.

107. (canceled)

108. The siNA molecule according to claim 106, wherein the ligand is

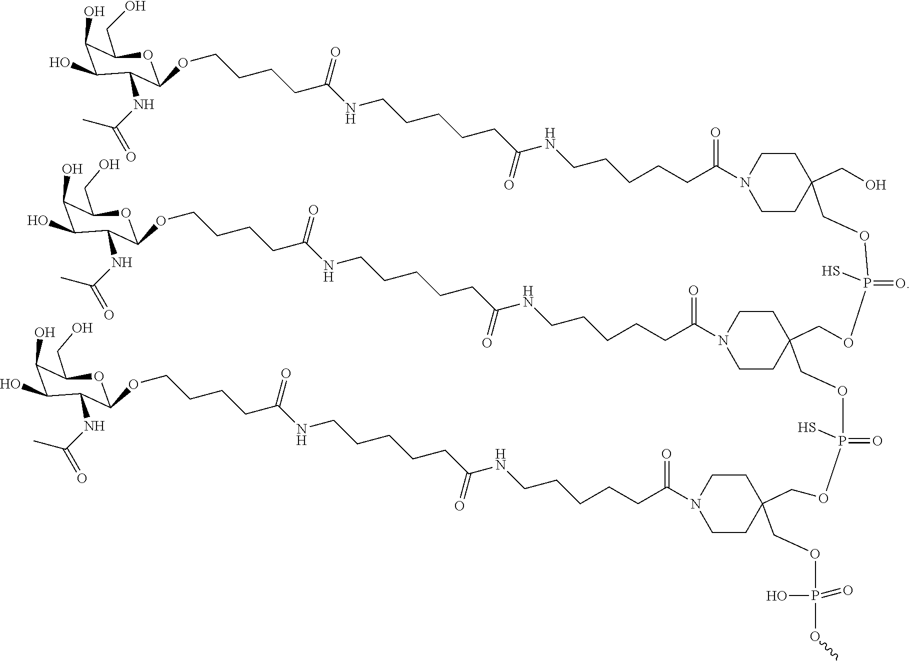

109. The siNA molecule according to claim 4, wherein the sense strand is selected from any one of SEQ ID NOs: 316-445, 576-603, or 638.

110. The siNA molecule according to claim 4, wherein the antisense strand is selected from any one of SEQ ID NOs: 446-575, 604-637 or 639-644.

111. A method of reducing the expression level of HSD17B13 in a subject in need thereof comprising administering to the subject an amount of the pharmaceutical composition according to claim 16, thereby reducing the expression level of HSD17B13 in the subject.

112. A method of preventing at least one symptom of a liver disease in a subject in need thereof comprising administering to the subject an amount of the pharmaceutical composition according to claim 16, thereby preventing at least one symptom of a liver disease in the subject.
